(19) United States
(12) Patent Application Publication
(10) Pub. No.: US 2025/0206722 A1
(43) Pub. Date: Jun. 26, 2025

(54) SMALL MOLECULE INHIBITORS OF TEA DOMAIN FAMILY MEMBERS

(71) Applicant: The Scripps Research Institute, La Jolla, CA (US)

(72) Inventors: Michael J. Bollong, San Diego, CA (US); Jian Jeffrey Chen, San Diego, CA (US); Lirui Song, San Diego, CA (US); Kayla Nutsch, San Diego, CA (US); Peter G. Schultz, La Jolla, CA (US); Arnab Chatterjee, San Diego, CA (US)

(21) Appl. No.: 18/848,311

(22) PCT Filed: Mar. 20, 2023
(86) PCT No.: PCT/US23/64697

§ 371 Date: Sep. 18, 2024

Related U.S. Application Data
(60) Provisional application No. 63/269,733, filed on Mar. 22, 2022.

(51) Int. Cl. C07D 403/04; A61K 31/454; A61K 31/4545; A61K 31/497; A61K 31/4985; A61K 31/4995; A61K 31/501; A61K 31/506; A61K 31/551; A61K 45/06; A61P 35/00; C07D 213/74; C07D 241/18; C07D 241/20; C07D 295/185; C07D 401/04; C07D 401/14; C07D 403/12; C07D 403/14; C07D 487/04; C07D 487/08

(52) U.S. Cl. CPC C07D 403/04; A61K 31/454; A61K 31/4545; A61K 31/497; A61K 31/4985; A61K 31/4995; A61K 31/501; A61K 31/506; A61K 31/551; A61K 45/06; A61P 35/00; C07D 213/74; C07D 241/18; C07D 241/20; C07D 295/185; C07D 401/04; C07D 401/14; C07D 403/12; C07D 403/14; C07D 487/04; C07D 487/08

(57) ABSTRACT

The present disclosure provides a compound of Formula (I): or a tautomer or pharmaceutically acceptable salt thereof, wherein Rings A, B, C, and R¹ and R² are defined herein, their pharmaceutical compositions, and methods of use for treating diseases that are vulnerable to inhibition of TEAD family transcription factors and/or inhibition of the pro-growth activity of the transcriptional coactivator YAP.

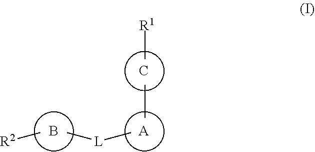

### CROSS-REFERENCE

This application claims the benefit of US Provisional Application No. 63/269,733, filed on Mar. 22, 2022, which is incorporated herein by reference in its entirety.

### BACKGROUND

Originally identified from screens for organ overgrowth in flies, the conserved Hippo pathway controls cellular proliferation, organ size, and sternness in most mammalian tissues. At its core, the Hippo pathway consists of linear kinase cascade, in which extracellular signals such as cellular density and substrate stiffness result in activation of the kinases MST1/2 (serine/threonine-protein kinase 4 (STK4, called MST1) and serine/threonine-protein kinase 3 (STK3, called MST2) and LATS1/2 (Large Tumor Suppressor Kinase 1 and Large Tumor Suppressor Kinase 2).2 Phosphoryl relay through MST1/2 and LATS1/2 result in the phosphorylation and cytoplasmic sequestration of Yes associated protein 1 (YAP1, called YAP throughout). YAP is a transcriptional co-activator that, through its interactions with TEA domain family proteins (TEADs: genes TEAD1, TEAD2, TEAD3, and TEAD4), promotes a pro-proliferative transcriptional program resulting in cellular expansion³.

Because YAP is central to growth control, several cancer types possess mutations that inactivate Hippo pathway members, resulting in constitutive activation of YAP and runaway cellular proliferation. ⁴ For example, YAP activating mutations are present in more than 70 percent of all malignant pleural mesotheliomas (MPMs). ⁵ YAP additionally promotes immune evasion in solid tumors by directly regulating the transcription of PD-L1 as well as TNF-α, CXCL6, CCL2, and CSF1, which promote M2 macrophage polarization and the accumulation of tumor resident myeloid-derived suppressor cells (MDSCs). ⁶⁻⁸ In addition to controlling ‘tumor intrinsic’ processes, YAP promotes activation of T regulatory cells (Tgs) in the tumor and suppresses the activation of CD4 and CD8 positive tumor reactive T cells. “Although these data indicate that inhibition of YAP is of high therapeutic utility in oncological and immuno-oncological indications, YAP does not possess a druggable protein domain.

### SUMMARY

To address these deficiencies and others, the present disclosure provides potent small molecule inhibitors to the TEA domain family proteins (TEADs), which are the DNA-binding transcription factors that allow loci specific localization of YAP at TEAD-binding elements in the genome”. Each of the inhibitors is a compound of formula (I), a tautomer, or a pharmaceutically acceptable salt thereof:

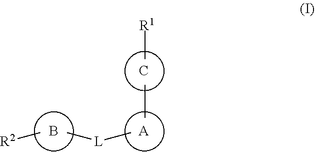

Ring A is a 6-membered aryl or 6-membered heteroaryl having 1, 2, or 3 nitrogen ring members.

Ring B is a C₆-C₁₂-aryl, C₄-C₇-cycloalkyl, or 6-membered heteroaryl having 1 or 2 nitrogen ring members.

Ring C is a 4- to 10-membered heterocycle containing 1 or 2 N ring members and that is optionally fused and optionally has 1 to 3 unsaturated bonds.

L is selected from the group consisting of —NH—, —O—, —CH₂—, —S—, —C(O)—, —CHOH—, and a bond.

In various embodiments, R¹ is bound to a carbon ring member, and R¹ is selected from the group consisting of CN, halo, —[CH₂]_{0,1}NHC(O)(C₁-C₆-alkyl), —[CH₂]_{0,1}NHC(O)(C₂-C₆-alkenyl), and —[CH₂]_{0,1}NHC(O)(C₂-C₆-alkynyl), —[CH₂]_{0,1}NHC(O)(C₃-C₁₀-cycloalkyl), —[CH₂]_{0,1}NHC(O)(3- to 6-membered heterocycloalkyl (wherein 1-4 ring members are independently selected from N, O, and S)), —[CH₂]_{0,1}NHSO₂(C₁-C₆-alkyl), —[CH₂]_{0,1}NHSO₂(C₂-C₆-alkenyl), and —[CH₂]_{0,1}NHSO₂(C₂-C₆-alkynyl), —[CH₂]_{0,1}NHSO₂(C₃-C₁₀-cycloalkyl), and —[CH₂]_{0,1}NHSO₂(3- to 6-membered heterocycloalkyl (wherein 1-4 ring members are independently selected from N, O, and S)).

In other embodiments, R¹ is bound to a nitrogen ring member, and R¹ is selected from the group consisting of CN, —C(O)(C₁-C₆-alkyl), —C(O)(C₂-C₆-alkenyl), —C(O)(C₂-C₆-alkynyl), —C(O)(C₃-C₁₀-cycloalkyl), —C(O)(3- to 6-membered heterocycloalkyl (wherein 1-4 ring members are independently selected from N, O, and S)), —C₁-C₆-alkyl(C₃-C₁₀-cycloalkyl), —SO₂(C₁-C₆-alkyl), —SO₂(C₂-C₆-alkenyl), —SO₂(C₂-C₆-alkynyl), —SO₂(C₃-C₁₀-cycloalkyl), and —SO₂(3- to 6-membered heterocycloalkyl (wherein 1-4 ring members are independently selected from N, O, and S)).

R² is selected from the group consisting of halo, C₁-C₆-haloalkyl, C₁-C₆-haloalkoxy, and —SF₅.

Ring A, Ring B, and Ring C are independently and optionally substituted with 1 to 3 three substituents independently selected from halo and C₁-C₆-alkyl.

In Formula (I), any alkyl, alkenyl, alkynyl, cycloalkyl, and heterocycloalkyl is optionally substituted with one to three substituents independently selected from the group consisting of halo, OH, CN, C₁-C₆-alkoxy, C₁-C₆-alkyl, C₁-C₆-haloalkyl, C₁-C₆-hydroxyalkyl, (C₀-C₆-alkyl)NRR′, —(C₀-C₆-alkyl)-OC(O)R, —(C₀-C₆-alkyl)-C(O)OR, C₃-C₁₀-cycloalkyl, 3- to 6-membered heterocycloalkyl (wherein 1-4 ring members are independently selected from N, O, and S), —(C₁-C₆-alkyl)(3- to 6-membered heterocycloalkyl (wherein 1-4 ring members are independently selected from N, O, and S).

R and R′ are independently selected from H, C₁-C₆-alkyl, C₂-C₆-alkenyl, and C₂-C₆-alkynyl.

The present disclosure also provides in embodiments a pharmaceutical composition comprising a compound or pharmaceutically acceptable salt thereof as described herein and a pharmaceutically acceptable carrier.

### DETAILED DESCRIPTION

The compounds of the present disclosure are potent inhibitors of TEADs and are therefore useful, in various embodiments, as therapeutics for treating oncological and immuno-oncological indications. Needs in the art reside in development of therapies that affect tumor intrinsic growth processes and that concurrently activate adaptive immune responses to the tumor. Compounds of the present disclosure target the TEAD family transcription factors and thereby fulfill both desired activities by inhibiting the pro-growth activity of the transcriptional coactivator YAP and by promoting activation of various tumor reactive T cell populations. The compounds are therefore of robust utility in treating various tumor types.

The compounds engender numerous advantages. First, in contrast to known TEADs inhibitors, Formula (I) compounds of the present disclosure are lower in molecular weight and less hydrophobic. In this context, some known covalent inhibitors feature electrophilic targeting groups (e.g., acrylamide) bound directly to aromatic moieties, which increases the electrophilicity of this covalent targeting group: this structural combination contributes to decreased selectivity of the inhibitors for TEADs. In contrast, compounds of the present disclosure in conformance with Formula (I) are less electrophilic. A second advantage resides in the surprising discovery that compounds of the present disclosure, as illustrated in the examples below, arrest cancer cell growth by selectively inhibiting TEADs: the use of a covalent drug to target TEADs is to increase the tolerability and safety of the compounds because lower doses of compound are required to inactivate TEADS, especially in view of long compound half lives in cells (e.g., >24 hours).

### EMBODIMENTS

Embodiment 1. A compound of Formula (I), or a pharmaceutically acceptable salt thereof:
wherein

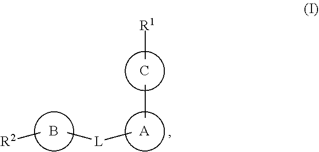

Ring A is a 6-membered aryl or 6-membered heteroaryl having 1, 2, or 3 nitrogen ring members,
Ring B is a C₆-C₁₂-aryl, C₄-C₇-cycloalkyl, or 6-membered heteroaryl having 1 or 2 nitrogen ring members;
Ring C is a 4- to 10-membered heterocycle containing 1 or 2 N ring members and that is optionally fused and optionally has 1 to 3 unsaturated bonds;
L is selected from the group consisting of —NH—, —O—, —CH₂—, —S—, —C(O)—, —CHOH—, and a bond;
   wherein
when R¹ is bound to a carbon ring member, then
R¹ is selected from the group consisting of CN, halo, —[CH₂]_{0,1}NHC(O)(C₁-C₆-alkyl), —[CH₂]_{0,1}NHC(O)(C₂-C₆-alkenyl), and —[CH₂]_{0,1}NHC(O)(C₂-C₆-alkynyl), —[CH₂]_{0,1}NHC(O)(C₃-C₁₀-cycloalkyl), —[CH₂]_{0,1}NHC(O)(3- to 6-membered heterocycloalkyl (wherein 1-4 ring members are independently selected from N, O, and S)), —[CH₂]_{0,1}NHSO₂(C₁-C₆-alkyl), —[CH₂]_{0,1}NHSO₂(C₂-C₆-alkenyl), and —[CH₂]_{0,1}NHSO₂(C₂-C₆-alkynyl), —[CH₂]_{0,1}NHSO₂(C₃-C₁₀-cycloalkyl), and —[CH₂]_{0,1}NHSO₂(3- to 6-membered heterocycloalkyl (wherein 1-4 ring members are independently selected from N, O, and S))
when R¹ is bound to a nitrogen ring member, then
R¹ is selected from the group consisting of CN, —C(O)(C₁-C₆-alkyl), —C(O)(C₂-C₆-alkenyl), —C(O)(C₂-C₆-alkynyl), —C(O)(C₃-C₁₀-cycloalkyl), —C(O)(3- to 6-membered heterocycloalkyl (wherein 1-4 ring members are independently selected from N, O, and S)), —C₁-C₆-alkyl(C₃-C₁₀-cycloalkyl), —SO₂(C₁-C₆-alkyl), —SO₂(C₂-C₆-alkenyl), —SO₂(C₂-C₆-alkynyl), —SO₂(C₃-C₁₀-cycloalkyl), and —SO₂(3- to 6-membered heterocycloalkyl (wherein 1-4 ring members are independently selected from N, O, and S));
R² is selected from the group consisting of halo, C₁-C₆-haloalkyl, C₁-C₆-haloalkoxy, and —SF₅;
Ring A, Ring B, and Ring C are independently and optionally substituted with 1 to 3 three substituents independently selected from halo and C₁-C₆-alkyl; and
any alkyl, alkenyl, alkynyl, cycloalkyl, and heterocycloalkyl is optionally substituted with one to three substituents independently selected from the group consisting of halo, OH, CN, C₁—C₆-alkoxy, C₁-C₆-alkyl, C₁-C₆-haloalkyl, C₁-C₆-hydroxyalkyl, (C₀-C₆-alkyl)NRR′, —(C₀-C₆-alkyl)-OC(O)R, —(C₀-C₆-alkyl)-C(O)OR, C₃-C₁₀-cycloalkyl, 3- to 6-membered heterocycloalkyl (wherein 1-4 ring members are independently selected from N, O, and S), —(C₁-C₆-alkyl)(3- to 6-membered heterocycloalkyl (wherein 1-4 ring members are independently selected from N, O, and S),
wherein R and R′ are independently selected from H, C₁-C₆-alkyl, C₂-C₆-alkenyl, and C₂-C₆-alkynyl).

Embodiment 2. The compound or pharmaceutically acceptable salt thereof according to embodiment 1, wherein Ring A is 6-membered heteroaryl having 2 or 3 nitrogen ring members.

Embodiment 3. The compound or pharmaceutically acceptable salt thereof according to embodiment 1 or 2, wherein Ring A is selected from the group consisting of optionally substituted:

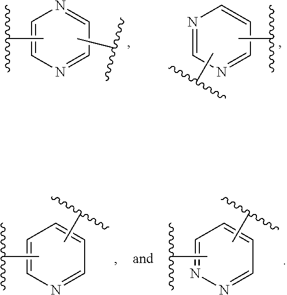

Embodiment 4. The compound or pharmaceutically acceptable salt thereof according to any one of embodiments 1 to 3, wherein Ring A is optionally substituted:

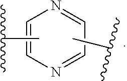

Embodiment 5. The compound or pharmaceutically acceptable salt thereof according to any one of embodiments 1 to 3, wherein Ring A is optionally substituted:

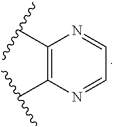 [SMILES: *c1nccnc1*]

Embodiment 6. The compound or pharmaceutically acceptable salt thereof according to any one of embodiments 1 to 5, wherein Ring B is optionally substituted C₆-C₁₂-aryl.

Embodiment 7. The compound or pharmaceutically acceptable salt thereof according to any one of embodiments 1 to 6, wherein Ring B is optionally substituted phenyl.

Embodiment 8. The compound or pharmaceutically acceptable salt thereof according to any one of embodiments 1 to 5, wherein Ring B is optionally substituted 6-membered heteroaryl having 1 or 2 nitrogen ring members.

Embodiment 9. The compound or pharmaceutically acceptable salt thereof according to any one of embodiments 1 to 5 and 8, wherein Ring B is selected from the group of optionally substituted:

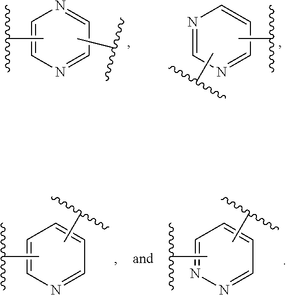

Embodiment 10. The compound or pharmaceutically acceptable salt thereof according to any one of embodiments 1 to 9, wherein Ring C is an optionally substituted 4- to 8-membered heterocycle.

Embodiment 11. The compound or pharmaceutically acceptable salt thereof according to any one of embodiments 1 to 10, wherein Ring C contains 1 N ring member.

Embodiment 12. The compound or pharmaceutically acceptable salt thereof according to any one of embodiments 1 to 10, wherein Ring C contains 2 N ring members.

Embodiment 13. The compound or pharmaceutically acceptable salt thereof according to any one of embodiments 1 to 10, wherein Ring C is selected from the group consisting of optionally substituted:

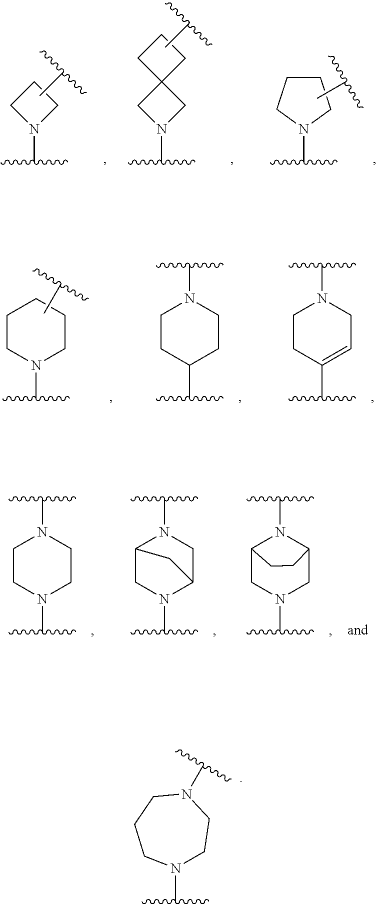

Embodiment 14. The compound or pharmaceutically acceptable salt thereof according to any one of embodiments 1 to 11, wherein Ring C is optionally substituted:

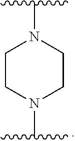 [SMILES: *N1CCN(*)CC1]

Embodiment 15. The compound or pharmaceutically acceptable salt thereof according to any one of embodiments 1 to 14, wherein L is —NH—.

Embodiment 16. The compound or pharmaceutically acceptable salt thereof according to any one of embodiments 1 to 15, wherein R² is C₁-C₆-haloalkyl.

Embodiment 17. The compound or pharmaceutically acceptable salt thereof according to any one of embodiments 1 to 16, wherein R² is —CF₃.

Embodiment 18. The compound or pharmaceutically acceptable salt thereof according to any one of embodiments 1 to 17, wherein R¹ is bound to a nitrogen ring member.

Embodiment 19. The compound or pharmaceutically acceptable salt thereof according to embodiment 18, wherein R¹ has the formula

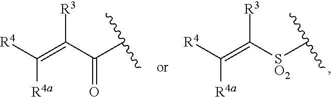

wherein R³, R⁴, and R^{4a} are individually selected from the group consisting of H, halo, CN, —C(O)OR, and optionally substituted C₁-C₆-alkyl.

Embodiment 20. The compound or pharmaceutically acceptable salt thereof according to embodiment 19, wherein R¹ has the formula:

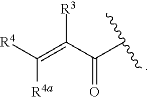

Embodiment 21. The compound or pharmaceutically acceptable salt thereof according to embodiment 19 or 20, wherein:
R³ is selected from the group consisting of H, F, Cl, CH₂F, CF₃, CN, CH₂OH, CH₂OC(O)R, and CH₂NRR′; and
R⁴ and R^{4a} are independently selected from the group consisting of H, CH₂F, CHF₂, CH₂OH, CH₂NRR′, and CH₂-(3- to 6-membered heterocycloalkyl (wherein 1-4 ring members are independently selected from N, O, and S).

Embodiment 22. The compound or pharmaceutically acceptable salt thereof according to embodiment 18, wherein R¹ has the formula:

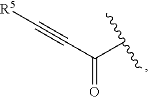

wherein R⁵ is selected from the group consisting of H, C₁-C₆-alkyl, C₁-C₆-haloalkyl, and C₁-C₆-hydroxyalkyl.

Embodiment 23. The compound or pharmaceutically acceptable salt thereof according to embodiment 18, wherein R¹ has the formula:

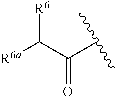

wherein R⁶ and R^{6a} are independently selected from the group consisting of H, halo, CN, and C₁-C₆-alkyl.

Embodiment 24. The compound or pharmaceutically acceptable salt thereof according to embodiment 23, wherein at least one of R⁶ and R^{6a} is halo.

Embodiment 25. The compound or pharmaceutically acceptable salt thereof according to embodiment 23 or 24, wherein each of R⁶ and R^{6a} is halo.

Embodiment 26. The compound or pharmaceutically acceptable salt thereof according to embodiment 23 or 24, wherein at least one of R⁶ and R^{6a} is CN.

Embodiment 27. The compound or pharmaceutically acceptable salt thereof according to embodiment 18, wherein R¹ has the formula:

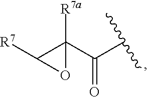

wherein R⁷ and R^{7a} are independently selected from the group consisting of H, optionally substituted alkyl, and —C(O)OR.

Embodiment 28. The compound or pharmaceutically acceptable salt thereof according to embodiment 1 or 18, wherein R¹ is selected from the group consisting of.

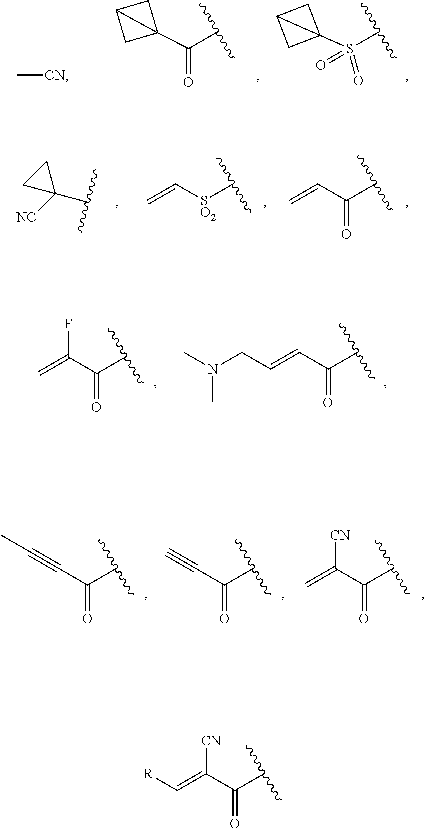

(wherein R is substituted alkyl),

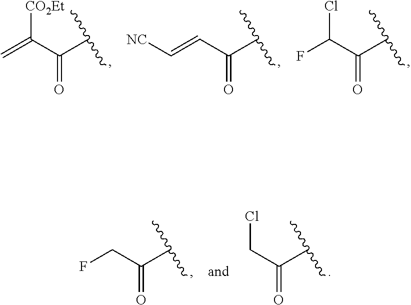

Embodiment 29. The compound or pharmaceutically acceptable salt thereof according to embodiment 1, 18, or 28, wherein R¹ is:

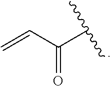 [SMILES: *C(=O)C=C]

Embodiment 30. The compound or pharmaceutically acceptable salt thereof according to embodiment 1, wherein the compound is of Formula (IA):

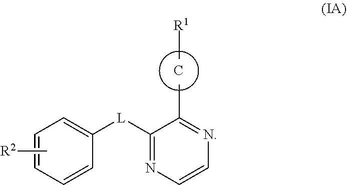

Embodiment 31. The compound or pharmaceutically acceptable salt thereof according to embodiment 30, wherein L is —NH—.

Embodiment 32. The compound or pharmaceutically acceptable salt thereof according to embodiment 30 or 31, wherein R² is —CF₃ or —SFS.

Embodiment 33. The compound or pharmaceutically acceptable salt thereof according to any one of embodiments 30 to 32, wherein R² is in the para position.

Embodiment 34. The compound or pharmaceutically acceptable salt thereof according to any one of embodiments 30 to 33, wherein Ring C is a 4- to 6-membered heterocycle containing 2 N ring members.

Embodiment 35. The compound or pharmaceutically acceptable salt thereof according to any one of embodiments 30 to 34, wherein Ring C is:

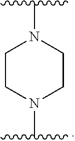 [SMILES: *N1CCN(*)CC1]

Embodiment 36. The compound or pharmaceutically acceptable salt thereof according to embodiment 1, wherein the compound is selected from the following:
1-(4-(3-((4-(trifluoromethyl)phenyl)amino)pyrazin-2-yl)piperazin-1-yl)prop-2-en-1-one;
4-(3-((4-(trifluoromethyl)phenyl)amino)pyrazin-2-yl)piperazine-1-carbonitrile;
N-(1-(3-((4-(trifluoromethyl)phenyl)amino)pyrazin-2-yl)azetidin-3-yl)acrylamide;
N-((1r,3r)-3-((3-((4-(trifluoromethyl)phenyl)amino)pyrazin-2-yl)amino)cyclobutyl) acrylamide;
N-((1s,3s)-3-((3-((4-(trifluoromethyl)phenyl)amino)pyrazin-2-yl)amino)cyclobutyl) acrylamide;
N-(2-(3-((4-(trifluoromethyl)phenyl)amino)pyrazin-2-yl)-2-azaspiro[3.3]heptan-6-yl)acrylamide;
N-(1-(3-((4-(trifluoromethyl)phenyl)amino)pyrazin-2-yl)pyrrolidin-3-yl)acrylamide;
1-(3-((3-((4-(trifluoromethyl)phenyl)amino)pyrazin-2-yl)amino)pyrrolidin-1-yl)prop-2-en-1-one;
N-(1-(3-((4-(trifluoromethyl)phenyl)amino)pyrazin-2-yl)piperidin-4-yl)acrylamide;
N-((1-(3-((4-(trifluoromethyl)phenyl)amino)pyrazin-2-yl)piperidin-3-yl)methyl)acrylamide;
1-(5-(3-((4-(trifluoromethyl)phenyl)amino)pyrazin-2-yl)-2,5-diazabicyclo[2.2.1]heptan-2-yl)prop-2-en-1-one;
1-((1R,5S)-3-(3-((4-(trifluoromethyl)phenyl)amino)pyrazin-2-yl)-3,8-diazabicyclo[3.2.1]octan-8-yl)prop-2-en-1-one;
(R)-1-(2-methyl-4-(3-((4-(trifluoromethyl)phenyl)amino)pyrazin-2-yl)piperazin-1-yl)prop-2-en-1-one;
(S)-1-(2-methyl-4-(3-((4-(trifluoromethyl)phenyl)amino)pyrazin-2-yl)piperazin-1-yl)prop-2-en-1-one;
(R)-1-(2-(hydroxymethyl)-4-(3-((4-(trifluoromethyl)phenyl)amino)pyrazin-2-yl)piperazin-1-yl)prop-2-en-1-one;
(S)-1-(2-(hydroxymethyl)-4-(3-((4-(trifluoromethyl)phenyl)amino)pyrazin-2-yl)piperazin-1-yl)prop-2-en-1-one;
(S)-1-(2-(2-hydroxyethyl)-4-(3-((4-(trifluoromethyl)phenyl)amino)pyrazin-2-yl)piperazin-1-yl)prop-2-en-1-one;
(R)-1-acryloyl-4-(3-((4-(trifluoromethyl)phenyl)amino)pyrazin-2-yl)piperazine-2-carboxamide;
(S)-1-acryloyl-4-(3-((4-(trifluoromethyl)phenyl)amino)pyrazin-2-yl)piperazine-2-carboxamide;
2-(1-acryloyl-4-(3-((4-(trifluoromethyl)phenyl)amino)pyrazin-2-yl)piperazin-2-yl)acetamide;
(R)-2-(1-acryloyl-4-(3-((4-(trifluoromethyl)phenyl)amino)pyrazin-2-yl)piperazin-2-yl)acetonitrile;
(S)-2-(1-acryloyl-4-(3-((4-(trifluoromethyl)phenyl)amino)pyrazin-2-yl)piperazin-2-yl)acetonitrile;
1-(2-(prop-2-yn-1-yl)-4-(3-((4-(trifluoromethyl)phenyl)amino)pyrazin-2-yl)piperazin-1-yl)prop-2-en-1-one;
1-(3-methyl-4-(3-((4-(trifluoromethyl)phenyl)amino)pyrazin-2-yl)piperazin-1-yl)prop-2-en-1-one;
1-[4-(3-{[4-(trifluoromethyl)phenyl]amino}pyrazin-2-yl)-3,6-dihydro-2H-pyridin-1-yl]prop-2-en-1-one;
1-[4-(3-{[4-(trifluoromethyl)phenyl]amino}pyrazin-2-yl)piperidin-1-yl]prop-2-en-1-one;
1-(4-(3-((4-(trifluoromethyl)phenyl)amino)pyrazin-2-yl)-1,4-diazepan-1-yl)prop-2-en-1-one;
1-(4-(3-(4-(trifluoromethyl)phenyl)pyrazin-2-yl)piperazin-1-yl)prop-2-en-1-one;
1-(4-(3-(3-(trifluoromethyl)phenyl)pyrazin-2-yl)piperazin-1-yl)prop-2-en-1-one;
1-(4-(3-((3-(trifluoromethyl)phenyl)amino)pyrazin-2-yl)piperazin-1-yl)prop-2-en-1-one;
1-(4-(3-((4-(pentafluoro-16-sulfaneyl)phenyl)amino)pyrazin-2-yl)piperazin-1-yl)prop-2-en-1-one;
1-(4-(3-((4-chlorophenyl)amino)pyrazin-2-yl)piperazin-1-yl)prop-2-en-1-one;
1-(4-(3-((4-fluorophenyl)amino)pyrazin-2-yl)piperazin-1-yl)prop-2-en-1-one;
1-(4-(3-((4-(trifluoromethoxy)phenyl)amino)pyrazin-2-yl)piperazin-1-yl)prop-2-en-1-one;
1-(4-(3-((4-(difluoromethoxy)phenyl)amino)pyrazin-2-yl)piperazin-1-yl)prop-2-en-1-one;
1-(4-(3-((4-(fluoromethoxy)phenyl)amino)pyrazin-2-yl)piperazin-1-yl)prop-2-en-1-one;
1-(4-(3-((3-methoxy-4-(trifluoromethyl)phenyl)amino)pyrazin-2-yl)piperazin-1-yl)prop-2-en-1-one;
4-((3-(4-acryloylpiperazin-1-yl)pyrazin-2-yl)amino)benzonitrile;
1-(4-(3-((5-(trifluoromethyl)pyridin-2-yl)amino)pyrazin-2-yl)piperazin-1-yl)prop-2-en-1-one;
1-(4-(3-((5-(difluoromethyl)pyridin-2-yl)amino)pyrazin-2-yl)piperazin-1-yl)prop-2-en-1-one;
1-(4-(3-((5-(trifluoromethyl)pyrimidin-2-yl)amino)pyrazin-2-yl)piperazin-1-yl)prop-2-en-1-one;
1-(4-(3-(methyl(5-(trifluoromethyl)pyridin-2-yl)amino)pyrazin-2-yl)piperazin-1-yl)prop-2-en-1-one;
1-(4-(3-((5-(trifluoromethyl)pyridin-2-yl)amino)pyrazin-2-yl)-3,6-dihydropyridin-1(2H)-yl)prop-2-en-1-one;
1-(4-(3-((5-(trifluoromethyl)pyridin-2-yl)amino)pyrazin-2-yl)piperidin-1-yl)prop-2-en-1-one;
1-(4-(3-((5-(trifluoromethyl)pyridin-2-yl)amino)pyrazin-2-yl)piperazin-1-yl)prop-2-en-1-one;
2-fluoro-1-(4-(3-((6-(trifluoromethyl)pyridin-3-yl)amino)pyrazin-2-yl)piperazin-1-yl)prop-2-en-1-one;
1-(4-(3-((6-(difluoromethyl)pyridin-3-yl)amino)pyrazin-2-yl)piperazin-1-yl)prop-2-en-1-one;
1-(4-(3-((6-(trifluoromethoxy)pyridin-3-yl)amino)pyrazin-2-yl)piperazin-1-yl)prop-2-en-1-one;
1-(4-(3-((6-(difluoromethoxy)pyridin-3-yl)amino)pyrazin-2-yl)piperazin-1-yl)prop-2-en-1-one;
1-(4-(3-((5-fluoro-6-(trifluoromethyl)pyridin-3-yl)amino)pyrazin-2-yl)piperazin-1-yl)prop-2-en-1-one;
2-fluoro-1-(4-(3-((5-fluoro-6-(trifluoromethyl)pyridin-3-yl)amino)pyrazin-2-yl)piperazin-1-yl)prop-2-en-1-one;
1-(4-(3-((2-(trifluoromethyl)pyrimidin-5-yl)amino)pyrazin-2-yl)piperazin-1-yl)prop-2-en-1-one;
(S)-1-(2-methyl-4-(3-((6-(trifluoromethyl)pyridin-3-yl)amino)pyrazin-2-yl)piperazin-1-yl)prop-2-en-1-one;
(R)-1-(2-methyl-4-(3-((6-(trifluoromethyl)pyridin-3-yl)amino)pyrazin-2-yl)piperazin-1-yl)prop-2-en-1-one;
(R)-1-(2-methyl-4-(3-((6-(trifluoromethyl)pyridin-3-yl)amino)pyrazin-2-yl)piperazin-1-yl)prop-2-en-1-one;
(R)-1-(2-methyl-4-(3-((6-(trifluoromethyl)pyridin-3-yl)amino)pyrazin-2-yl)piperazin-1-yl)prop-2-en-1-one;
(R)-1-(2-methyl-4-(3-((6-(trifluoromethyl)pyridin-3-yl)amino)pyrazin-2-yl)piperazin-1-yl)prop-2-en-1-one;
2-fluoro-1-(4-(3-((6-(trifluoromethoxy)pyridin-3-yl)amino)pyrazin-2-yl)-3,6-dihydropyridin-1(2H)-yl)prop-2-en-1-one;
2-fluoro-1-(4-(3-((6-(trifluoromethoxy)pyridin-3-yl)amino)pyrazin-2-yl)-3,6-dihydropyridin-1(2H)-yl)prop-2-en-1-one;
2-fluoro-1-(4-(3-((2-(trifluoromethyl)pyrimidin-5-yl)amino)pyrazin-2-yl)-3,6-dihydropyridin-1(2H)-yl)prop-2-en-1-one;
1-(4-(3-((6-(trifluoromethyl)pyridin-3-yl)amino)pyrazin-2-yl)piperidin-1-yl)prop-2-en-1-one;
1-(3-((3-((6-(trifluoromethyl)pyridin-3-yl)amino)pyrazin-2-yl)amino)azetidin-1-yl)prop-2-en-1-one;
2-fluoro-1-(3-((3-((6-(trifluoromethyl)pyridin-3-yl)amino)pyrazin-2-yl)amino)azetidin-1-yl)prop-2-en-1-one;
2-fluoro-1-(3-((3-((6-(trifluoromethoxy)pyridin-3-yl)amino)pyrazin-2-yl)amino)azetidin-1-yl)prop-2-en-1-one;
2-fluoro-1-(3-((3-((6-(trifluoromethyl)pyridin-3-yl)amino)pyrazin-2-yl)oxy)azetidin-1-yl)prop-2-en-1-one;
1-(4-(3-((4,4-difluorocyclohexyl)amino)pyrazin-2-yl)piperazin-1-yl)prop-2-en-1-one;
1-(4-(3-((4,4-difluorocyclohexyl)amino)pyrazin-2-yl)piperazin-1-yl)prop-2-en-1-one;
2-fluoro-1-(4-(3-((4-(trifluoromethyl)bicyclo[1.1.1]pentan-2-yl)amino)pyrazin-2-yl)piperazin-1-yl)prop-2-en-1-one;
1-{4-[3-(4-tert-butylphenoxy)pyrazin-2-yl]piperazin-1-yl}prop-2-en-1-one;
1-(4-(3-(4-methoxyphenoxy)pyrazin-2-yl)piperazin-1-yl)prop-2-en-1-one;
1-(4-(3-(4-(difluoromethoxy)phenoxy)pyrazin-2-yl)piperazin-1-yl)prop-2-en-1-one;
1-(4-(3-(4-(trifluoromethoxy)phenoxy)pyrazin-2-yl)piperazin-1-yl)prop-2-en-1-one;
1-(4-(3-((5-(trifluoromethyl)pyridin-2-yl)oxy)pyrazin-2-yl)piperazin-1-yl)prop-2-en-1-one;
1-(4-(3-((5-(trifluoromethyl)pyridin-2-yl)oxy)pyrazin-2-yl)-3,6-dihydropyridin-1(2H)-yl)prop-2-en-1-one;
1-(4-(3-((5-(trifluoromethyl)pyridin-2-yl)oxy)pyrazin-2-yl)-3,6-dihydropyridin-1(2H)-yl)prop-2-en-1-one;
2-fluoro-1-(4-(3-((5-(trifluoromethyl)pyridin-2-yl)oxy)pyrazin-2-yl)piperidin-1-yl)prop-2-en-1-one;
1-(4-(3-((6-(trifluoromethyl)pyridin-3-yl)oxy)pyrazin-2-yl)piperazin-1-yl)prop-2-en-1-one;
2-fluoro-1-(4-(3-((6-(trifluoromethyl)pyridin-3-yl)oxy)pyrazin-2-yl)piperazin-1-yl)prop-2-en-1-one;
1-(4-(3-((6-(trifluoromethyl)pyridin-3-yl)oxy)pyrazin-2-yl)piperidin-1-yl)prop-2-en-1-one;
2-fluoro-1-(4-(3-((6-(trifluoromethyl)pyridin-3-yl)oxy)pyrazin-2-yl)piperidin-1-yl)prop-2-en-1-one;
2-(trifluoromethyl)-1-(4-(3-((6-(trifluoromethyl)pyridin-3-yl)oxy)pyrazin-2-yl)piperidin-1-yl)prop-2-en-1-one;
2-((4-(3-((6-(trifluoromethyl)pyridin-3-yl)oxy)pyrazin-2-yl)piperidin-1-yl)methyl)acrylic acid;
1-(4-hydroxy-4-(3-((6-(trifluoromethyl)pyridin-3-yl)oxy)pyrazin-2-yl)piperidin-1-yl)prop-2-en-1-one;
1-(4-(3-((6-(trifluoromethyl)pyridin-3-yl)oxy)pyrazin-2-yl)-3,6-dihydropyridin-1(2H)-yl)prop-2-en-1-one;
2-fluoro-1-(4-(3-((6-(trifluoromethyl)pyridin-3-yl)oxy)pyrazin-2-yl)-3,6-dihydropyridin-1(2H)-yl)prop-2-en-1-one;
2-((3-(3-((6-(trifluoromethyl)pyridin-3-yl)oxy)pyrazin-2-yl)azetidin-1-yl)methyl)acrylic acid;
1-(4-(3-((2-(trifluoromethyl)pyrimidin-5-yl)oxy)pyrazin-2-yl)piperazin-1-yl)prop-2-en-1-one;
2-fluoro-1-(4-(3-((2-(trifluoromethyl)pyrimidin-5-yl)oxy)pyrazin-2-yl)piperazin-1-yl)prop-2-en-1-one;
1-(4-(3-((2-(trifluoromethyl)pyrimidin-5-yl)oxy)pyrazin-2-yl)-3,6-dihydropyridin-1(2H)-yl)prop-2-en-1-one;
2-fluoro-1-(4-(3-((2-(trifluoromethyl)pyrimidin-5-yl)oxy)pyrazin-2-yl)-3,6-dihydropyridin-1(2H)-yl)prop-2-en-1-one;
1-(4-(3-((2-(trifluoromethyl)pyrimidin-5-yl)oxy)pyrazin-2-yl)piperidin-1-yl)prop-2-en-1-one;
2-fluoro-1-(4-(3-((2-(trifluoromethyl)pyrimidin-5-yl)oxy)pyrazin-2-yl)piperidin-1-yl)prop-2-en-1-one;
1-(4-{3-[(4,4-difluorocyclohexyl)oxy]pyrazin-2-yl}piperazin-1-yl) prop-2-en-1-one;
1-(4-(3-((4-(trifluoromethyl)cyclohexyl)oxy)pyrazin-2-yl)piperazin-1-yl)prop-2-en-1-one;
1-(4-(3-(4-(trifluoromethyl)benzoyl)pyrazin-2-yl)piperazin-1-yl)prop-2-en-1-one;
1-(4-(3-(6-(trifluoromethyl)nicotinoyl)pyrazin-2-yl)piperazin-1-yl)prop-2-en-1-one;
1-(4-(3-(hydroxy(4-(trifluoromethyl)phenyl)methyl)pyrazin-2-yl)piperazin-1-yl)prop-2-en-1-one;
1-(4-(3-(4-(trifluoromethyl)benzyl)pyrazin-2-yl)piperazin-1-yl)prop-2-en-1-one;
3-(4-acryloylpiperazin-1-yl)-N-cyclobutylpyrazine-2-carboxamide;
3-(4-acryloylpiperazin-1-yl)-N-(3,3-difluorocyclobutyl)pyrazine-2-carboxamide;
1-(4-(3-(6-(trifluoromethyl)-2-azaspiro[3.3]heptane-2-carbonyl)pyrazin-2-yl)piperazin-1-yl)prop-2-en-1-one;
3-(4-acryloylpiperazin-1-yl)-N-cyclopentylpyrazine-2-carboxamide;
1-(4-(3-(4-(trifluoromethyl)piperidine-1-carbonyl)pyrazin-2-yl)piperazin-1-yl)prop-2-en-1-one;
1-(4-(3-(4,4-difluoropiperidine-1-carbonyl)pyrazin-2-yl)piperazin-1-yl)prop-2-en-1-one;
1-(4-(3-(4-(trifluoromethyl)piperidin-1-yl)pyrazin-2-yl)piperazin-1-yl)prop-2-en-1-one;
1-(4-(3-(3-phenylazetidin-1-yl)pyrazin-2-yl)piperazin-1-yl)prop-2-en-1-one;
1-(4-(3-(3-(4-fluorophenyl)azetidin-1-yl)pyrazin-2-yl)piperazin-1-yl)prop-2-en-1-one;
1-(3-((3-(3-(4-fluorophenyl)azetidin-1-yl)pyrazin-2-yl)amino)azetidin-1-yl)prop-2-en-1-one;
2-fluoro-1-(3-((3-(3-(4-fluorophenyl)azetidin-1-yl)pyrazin-2-yl)amino)azetidin-1-yl)prop-2-en-1-one;
1-(4-(3-(6-(trifluoromethyl)-2-azaspiro[3.3]heptan-2-yl)pyrazin-2-yl)piperazin-1-yl)prop-2-en-1-one;
1-(4-(5-hydroxy-3-((4-(trifluoromethyl)phenyl)amino)pyrazin-2-yl)piperazin-1-yl)prop-2-en-1-one;
1-(4-(6-methyl-3-((4-(trifluoromethyl)phenyl)amino)pyrazin-2-yl)piperazin-1-yl)prop-2-en-1-one;
1-(4-(5,6-dimethyl-3-((6-(trifluoromethyl)pyridin-3-yl)amino)pyrazin-2-yl)piperazin-1-yl)prop-2-en-1-one;
1-(4-(5,6-dimethyl-3-((6-(trifluoromethyl)pyridin-3-yl)amino)pyrazin-2-yl)piperazin-1-yl)-2-fluoroprop-2-en-1-one;
2-fluoro-1-(4-(6-methyl-3-((6-(trifluoromethyl)pyridin-3-yl)oxy)pyrazin-2-yl)piperidin-1-yl)prop-2-en-1-one;
1-(4-(5-methyl-3-((6-(trifluoromethyl)pyridin-3-yl)oxy)pyrazin-2-yl)piperidin-1-yl)prop-2-en-1-one;
2-fluoro-1-(4-(5-methyl-3-((6-(trifluoromethyl)pyridin-3-yl)oxy)pyrazin-2-yl)piperidin-1-yl)prop-2-en-1-one;
3-(1-acryloylpiperidin-4-yl)-1-(5-(trifluoromethyl)pyrimidin-2-yl)pyrazin-2(1H)-one;
1-(4-(3-((4-(trifluoromethyl)phenyl)amino)pyridazin-4-yl)piperazin-1-yl)prop-2-en-1-one;
1-(4-(3-((5-(trifluoromethyl)pyridin-2-yl)amino)pyridazin-4-yl)piperazin-1-yl)prop-2-en-1-one;
1-(4-(3-((6-(trifluoromethyl)pyridin-3-yl)amino)pyridazin-4-yl)piperazin-1-yl)prop-2-en-1-one;
1-(4-(5-((4-(trifluoromethyl)phenyl)amino)pyridazin-4-yl)piperazin-1-yl)prop-2-en-1-one;
1-[4-(2-{[4-(trifluoromethyl)phenyl]amino}pyrimidin-4-yl)piperazin-1-yl]prop-2-en-1-one;
1-(4-(methyl(2-((4-(trifluoromethyl)phenyl)amino)pyrimidin-4-yl)amino)piperidin-1-yl)prop-2-en-1-one;
1-[4-(4-{[4-(trifluoromethyl)phenyl]amino}pyrimidin-2-yl)piperazin-1-yl]prop-2-en-1-one;
1-(4-(4-((6-(trifluoromethyl)pyridin-3-yl)oxy)pyrimidin-5-yl)piperidin-1-yl)prop-2-en-1-one;
2-fluoro-1-(4-(4-((6-(trifluoromethyl)pyridin-3-yl)oxy)pyrimidin-5-yl)piperidin-1-yl)prop-2-en-1-one;
1-(4-(2-methyl-5-((4-(trifluoromethyl)phenyl)amino)pyrimidin-4-yl)piperazin-1-yl)prop-2-en-1-one;
1-(4-(2-methyl-4-((6-(trifluoromethyl)pyridin-3-yl)oxy)pyrimidin-5-yl)-3,6-dihydropyridin-1(2H)-yl)prop-2-en-1-one;
2-fluoro-1-(4-(2-methyl-4-((6-(trifluoromethyl)pyridin-3-yl)oxy)pyrimidin-5-yl)-3,6-dihydropyridin-1(2H)-yl)prop-2-en-1-one;
1-(4-(2-methyl-4-((6-(trifluoromethyl)pyridin-3-yl)oxy)pyrimidin-5-yl)piperidin-1-yl)prop-2-en-1-one;
2-fluoro-1-(4-(2-methyl-4-((6-(trifluoromethyl)pyridin-3-yl)oxy)pyrimidin-5-yl)piperidin-1-yl)prop-2-en-1-one;
1-(4-(2-methoxy-5-((4-(trifluoromethyl)phenyl)amino)pyrimidin-4-yl)piperazin-1-yl)prop-2-en-1-one;
1-[4-(3-{[4-(trifluoromethyl)phenyl]amino}pyridin-2-yl)piperazin-1-yl]prop-2-en-1-one;
2-fluoro-1-(4-(3-((6-(trifluoromethyl)pyridin-3-yl)amino)pyridin-2-yl)piperazin-1-yl)prop-2-en-1-one;
1-(3-((6-(trifluoromethyl)pyridin-3-yl)oxy)-3′,6′-dihydro-[2,4′-bipyridin]-1′(2′H)-yl)prop-2-en-1-one;
1-(3-((6-(trifluoromethyl)pyridin-3-yl)oxy)-3′,6′-dihydro-[2,4′-bipyridin]-1′(2′H)-yl)prop-2-en-1-one;
1-(4-(3-((6-(trifluoromethyl)pyridin-3-yl)oxy)pyridin-2-yl)piperidin-1-yl)prop-2-en-1-one
2-fluoro-1-(4-(3-((6-(trifluoromethyl)pyridin-3-yl)oxy)pyridin-2-yl)piperidin-1-yl)prop-2-en-1-one;
1-(4-(3-(5-(trifluoromethyl)picolinoyl)pyridin-2-yl)piperazin-1-yl)prop-2-en-1-one;
1-(4-(3-(6-(trifluoromethyl)nicotinoyl)pyridin-2-yl)piperazin-1-yl)prop-2-en-1-one;
1-(4-(3-(hydroxy(6-(trifluoromethyl)pyridin-3-yl)methyl)pyridin-2-yl)piperazin-1-yl)prop-2-en-1-one;
1-[4-(2-{[4-(trifluoromethyl)phenyl]amino}pyridin-3-yl)piperazin-1-yl]prop-2-en-1-one;
of 2-fluoro-1-(4-(2-((6-(trifluoromethyl)pyridin-3-yl)amino)pyridin-3-yl)piperazin-1-yl)prop-2-en-1-one;
1-(2-((6-(trifluoromethyl)pyridin-3-yl)amino)-3′,6′-dihydro-[3,4′-bipyridin]-1′(2′H)-yl)prop-2-en-1-one;
2-fluoro-1-(2-((6-(trifluoromethyl)pyridin-3-yl)amino)-3′,6′-dihydro-[3,4′-bipyridin]-1′(2′H)-yl)prop-2-en-1-one;
1-(2-((6-(trifluoromethoxy)pyridin-3-yl)amino)-3′,6′-dihydro-[3,4′-bipyridin]-1′(2′H)-yl)prop-2-en-1-one;
2-fluoro-1-(2-((6-(trifluoromethoxy)pyridin-3-yl)amino)-3′,6′-dihydro-[3,4′-bipyridin]-1′(2′H)-yl)prop-2-en-1-one;
1-(4-(2-((6-(trifluoromethyl)pyridin-3-yl)amino)pyridin-3-yl)piperidin-1-yl)prop-2-en-1-one;
2-fluoro-1-(4-(2-((6-(trifluoromethyl)pyridin-3-yl)amino)pyridin-3-yl)piperidin-1-yl)prop-2-en-1-one;
1-(4-(2-((6-(trifluoromethoxy)pyridin-3-yl)amino)pyridin-3-yl)piperidin-1-yl)prop-2-en-1-one;
2-fluoro-1-(4-(2-((6-(trifluoromethoxy)pyridin-3-yl)amino)pyridin-3-yl)piperidin-1-yl)prop-2-en-1-one;
1-(2-((6-(trifluoromethyl)pyridin-3-yl)oxy)-3′,6′-dihydro-[3,4′-bipyridin]-1′(2′H)-yl)prop-2-en-1-one;
2-fluoro-1-(2-((6-(trifluoromethyl)pyridin-3-yl)oxy)-3′,6′-dihydro-[3,4′-bipyridin]-1′(2′H)-yl)prop-2-en-1-one;
1-(4-(2-((6-(trifluoromethyl)pyridin-3-yl)oxy)pyridin-3-yl)piperidin-1-yl)prop-2-en-1-one;
2-fluoro-1-(4-(2-((6-(trifluoromethyl)pyridin-3-yl)oxy)pyridin-3-yl)piperidin-1-yl)prop-2-en-1-one;
1-(2-((2-(trifluoromethyl)pyrimidin-5-yl)oxy)-3′,6′-dihydro-[3,4′-bipyridin]-1′(2′H)-yl)prop-2-en-1-one;
2-fluoro-1-(2-((2-(trifluoromethyl)pyrimidin-5-yl)oxy)-3′,6′-dihydro-[3,4′-bipyridin]-1′(2′H)-yl)prop-2-en-1-one;
1-(4-(2-((2-(trifluoromethyl)pyrimidin-5-yl)oxy)pyridin-3-yl)piperidin-1-yl)prop-2-en-1-one;
2-fluoro-1-(4-(2-((2-(trifluoromethyl)pyrimidin-5-yl)oxy)pyridin-3-yl)piperidin-1-yl)prop-2-en-1-one;
1-(4-(3-(4,4-difluoropiperidine-1-carbonyl)pyridin-2-yl)piperazin-1-yl)prop-2-en-1-one;
1-(4-(2-((4-(trifluoromethyl)phenyl)amino)phenyl)piperazin-1-yl)prop-2-en-1-one;
1-(4-(3-(4-(trifluoromethyl)benzoyl)-1H-pyrazol-1-yl)piperidin-1-yl)prop-2-en-1-one;
1-(4-(5-(4-(trifluoromethyl)benzoyl)-1H-pyrazol-1-yl)piperidin-1-yl)prop-2-en-1-one;
1-(4-(5-methyl-3-(4-(trifluoromethyl)benzoyl)-1H-pyrazol-1-yl)piperidin-1-yl)prop-2-en-1-one;
1-(4-(3-methyl-5-(4-(trifluoromethyl)benzoyl)-1H-pyrazol-1-yl)piperidin-1-yl)prop-2-en-1-one;
4-(dimethylamino)-1-(4-(3-((4-(trifluoromethyl)phenyl)amino)pyrazin-2-yl)piperazin-1-yl)but-2-en-1-one;
1-(4-(3-((4-(trifluoromethyl)phenyl)amino)pyrazin-2-yl)piperazin-1-yl)ethan-1-one;
2-chloro-2-fluoro-1-(4-(3-((4-(trifluoromethyl)phenyl)amino)pyrazin-2-yl)piperazin-1-yl)ethan-1-one;
2-chloro-1-(4-(3-((4-(trifluoromethyl)phenyl)amino)pyrazin-2-yl)piperazin-1-yl)ethan-1-one;
3-oxo-3-(4-(3-((4-(trifluoromethyl)phenyl)amino)pyrazin-2-yl)piperazin-1-yl)propanenitrile;
oxiran-2-yl(4-(3-((4-(trifluoromethyl)phenyl)amino)pyrazin-2-yl)piperazin-1-yl)methanone;
1-(4-(3-((4-(trifluoromethyl)phenyl)amino)pyrazin-2-yl)piperazin-1-yl)prop-2-yn-1-one;
1-(4-(3-((4-(trifluoromethyl)phenyl)amino)pyrazin-2-yl)piperazin-1-yl)but-2-yn-1-one;
2-(4-(3-((4-(trifluoromethyl)phenyl)amino)pyrazin-2-yl)piperazin-1-yl)acetonitrile;
2-methyl-2-(4-(3-((4-(trifluoromethyl)phenyl)amino)pyrazin-2-yl)piperazin-1-yl)propanenitrile;
2-methyl-1-(4-(3-((6-(trifluoromethyl)pyridin-3-yl)amino)pyrazin-2-yl)piperazin-1-yl)prop-2-en-1-one;
(E)-1-(4-(3-((6-(trifluoromethyl)pyridin-3-yl)amino)pyrazin-2-yl)piperazin-1-yl)but-2-en-1-one;
(E)-4-(methyl(prop-2-yn-1-yl)amino)-1-(4-(3-((4-(trifluoromethyl)phenyl)amino)pyrazin-2-yl)piperazin-1-yl)but-2-en-1-one;
2,3-dihydroxy-1-(4-(3-((4-(trifluoromethyl)phenyl)amino)pyrazin-2-yl)piperazin-1-yl)propan-1-one;
(E)-4-oxo-4-(4-(3-((4-(trifluoromethyl)phenyl)amino)pyrazin-2-yl)piperazin-1-yl)but-2-enenitrile;
(Z)-4-oxo-4-(4-(3-((4-(trifluoromethyl)phenyl)amino)pyrazin-2-yl)piperazin-1-yl)but-2-enenitrile;
(E)-4-oxo-4-(4-(3-((6-(trifluoromethyl)pyridin-3-yl)amino)pyrazin-2-yl)piperazin-1-yl)but-2-enenitrile;
(Z)-4-oxo-4-(4-(3-((6-(trifluoromethyl)pyridin-3-yl)amino)pyrazin-2-yl)piperazin-1-yl)but-2-enenitrile;
N-cyano-N-methyl-4-(3-((4-(trifluoromethyl)phenyl)amino)pyrazin-2-yl)piperazine-1-carboxamide;
N-cyano-N-methyl-4-(3-((4-(trifluoromethyl)phenyl)amino)pyrazin-2-yl)piperazine-1-carboxamide;
2-(4-(3-((4-(trifluoromethyl)phenyl)amino)pyrazin-2-yl)piperazine-1-carbonyl)allyl acetate;
2-(fluoromethyl)-1-(4-(3-((4-(trifluoromethyl)phenyl)amino)pyrazin-2-yl)piperazin-1-yl)prop-2-en-1-one;
2-(pyrrolidin-1-ylmethyl)-1-(4-(3-((4-(trifluoromethyl)phenyl)amino)pyrazin-2-yl)piperazin-1-yl)prop-2-en-1-one;
2-(trifluoromethoxy)-1-(4-(3-((4-(trifluoromethyl)phenyl)amino)pyrazin-2-yl)piperazin-1-yl)ethan-1-one;
N-methyl-N-(2-((3-((4-(trifluoromethyl)phenyl)amino)pyrazin-2-yl)oxy)ethyl)acrylamide;
N-(2-((3-((4-(trifluoromethyl)phenyl)amino)pyrazin-2-yl)oxy)ethyl)acrylamide;
N-(prop-2-yn-1-yl)-N-(2-((3-((4-(trifluoromethyl)phenyl)amino)pyrazin-2-yl)oxy)ethyl)acrylamide; and
N-((4-(6-(trifluoromethyl)pyridin-3-yl)-1,2,3,4-tetrahydropyrazino[2,3-b]pyrazin-2-yl)methyl)acrylamide.

Embodiment 37. A pharmaceutical composition comprising a compound or pharmaceutically acceptable salt thereof according to any one of embodiments 1 to 36 and a pharmaceutically acceptable carrier.

Embodiment 38. A method for treating a disease in a subject suffering therefrom, comprising administering to the subject a compound or pharmaceutically acceptable salt thereof according to any one of embodiments 1 to 36, wherein the disease is selected from the group consisting of Diabetic foot ulcer (DFU), Venous Ulcer (Stasis Ulcer), Pressure Ulcers, Full or partial thickness burns, Eczema, Psoriasis, Cellulitis, Impetigo, Atopic dermatitis, Epidermolysis Bullosa, Lichen Sclerosis, Ichthyosis, Vitiligo, Acral peeling skin syndrome, Blau syndrome, Primary cutaneous amyloidosis, Cutaneous abscess, Blepharitis, Furunculosis, Capillaritis, Cellulitis, Corneal Abrasion, Corneal Erosion, Xerosis, Lichen Planus, Lichen Simplex Chronicus, Idiopathic pulmonary fibrosis (IPF), Acute respiratory distress syndrome (ARDS), Chronic Obstructive Pulmonary Disease (COPD), Emphysema, Silicosis, Asbestosis, Pneumoconiosis, Aluminosis, Bauxite fibrosis, Berylliosis, Siderosis, Stannosis, Pulmonary Talcosis, Labrador lung (mixed dust Pneumoconiosis), Sarcoidosis, Hypersensitivity pneumonitis (HP)/extrinsic allergic alveolitis (EAA), Desquamative interstitial pneumonia (DIP), Respiratory bronchiolitis interstitial lung disease (RBILD), Acute interstitial pneumonia (AIP), Nonspecific interstitial pneumonia (NSIP), Cryptogenic organizing pneumonia (COP=idiopathic BOOP), Secondary organizing pneumonia (BOOP), Lymphoid interstitial pneumonia (LIP), Idiopathic interstitial pneumonia. unspecified, Hypereosinophilic lung diseases, Tuberculosis (TB), Pulmonary Edema, Interstitial Lung Disease, Cryptogenic Organizing Pneumonia (COP), E-cigarette or Vaping Use-Associated Lung Injury (EVALI), Hantavirus Pulmonary Syndrome (HPS), Histoplasmosis, Legionnaires' Disease, MAC Lung Disease, Alpha-1 Antitrypsin Deficiency, Aspergillosis, Lymphangioleiomyomatosis (LAM), Middle Eastern Respiratory Syndrome (MERS), Nontuberculous Mycobacterial Lung Disease (NTM), Pulmonary Embolism Goodpasture syndrome, idiopathic pulmonary hemosiderosis, Alveolar proteinosis, Pulmonary amyloidosis, Primary pulmonary lymphoma, Primary ciliary dyskinesia (without or with situs inversus), Rare cause of hypersensitivity pneumonitis (all causes other than farmer's lung disease and pigeon breeder's lung disease), Pulmonary arteriovenous malformations in hereditary hemorrhagic telangiectasia (HHT), interstitial lung disease in systemic sclerosis, interstitial lung disease in rheumatoid arthritis, interstitial lung disease in idiopathic inflammatory myopathies (polymyositis, dermatomyositis, anti-synthetase syndrome), interstitial lung disease in Sjögren syndrome, interstitial lung disease in mixed connective tissue disease (MCTD), interstitial lung disease in overlap syndromes, interstitial lung disease in undifferentiated connective tissue disease, Bronchiolitis obliterans (in non-transplanted patients), Infectious colitis, Ulcerative colitis, Crohn's disease, Ischemic colitis, Radiation colitis, Peptic ulcer, Intestinal cancer, Intestinal obstruction, Rheumatoid arthritis, Psoriatic arthritis, Hashimoto thyroiditis, Systemic lupus erythematosus, Multiple Sclerosis, Graves' Disease, Type 1 Diabetes Mellitus, Psoriasis, Ankylosing spondylitis, Scleroderma, Myositis, Gout, Antiphospholipid Antibody Syndrome (APS), Vasculitis, Dilated cardiomyopathy, Hypertrophic cardiomyopathy, Restrictive cardiomyopathy, Systolic heart failure, Diastolic heart failure (heart failure with preserved ejection fraction), Atrial Septal Defect, Atrioventricular Septal Defect, Coarctation of the Aorta, Double-outlet Right Ventricle, d-Transposition of the Great Arteries, Ebstein Anomaly, Hypoplastic Left Heart Syndrome, Interrupted Aortic Arch, Pulmonary Atresia, Single Ventricle, Tetralogy of Fallot, Total Anomalous Pulmonary Venous Return, Tricuspid Atresia, Truncus Arteriosus, Ventricular Septal Defect, Polycystic kidney disease, Diabetes Insipidus, Goodpasture's Disease, IgA Vasculitis, IgA Nephropathy, Lupus Nephritis, Adult Nephrotic Syndrome, Childhood Nephrotic Syndrome, Hemolytic Uremic Syndrome, Medullary Sponge Kidney, Kidney dysplasia, Renal artery stenosis, Renovascular hypertension, Renal tubular acidosis, Alport syndrome, Wenger's granulomatosis, Alagille syndrome, Cystinosis, Fabry disease, Focal segmental glomerulosclerosis (FSGS), Glomerulonephritis, aHUS (atypical hemolytic uremic syndrome), Hemolytic uremic syndrome (HUS), Henoch-Schonlein purpura, IgA nephropathy (Berger's disease), Interstitial nephritis, Minimal change disease, Nephrotic syndrome, Thrombotic thrombocytopenic purpura (TTP), Granulomatosis with polyangiitis (GPA), Adult Still's disease, Agammaglobulinemia, Alopecia areata, Autoimmune angioedema, Autoimmune dysautonomia, Autoimmune encephalomyelitis, Autoimmune hepatitis, Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Bullous pemphigoid, Celiac disease, Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Coxsackie myocarditis, CREST syndrome, Dermatitis herpetiformis, Dermatomyositis, Devic's disease (neuromyelitis optica), Discoid lupus, Eosinophilic esophagitis (EoE), Eosinophilic fasciitis, Erythema nodosum, Essential mixed cryoglobulinemia, Giant cell arteritis (temporal arteritis), Giant cell myocarditis, Granulomatosis with Polyangiitis, Guillain-Barre syndrome, Hashimoto's thyroiditis, Henoch-Schonlein purpura (HSP), Herpes gestationis or pemphigoid gestationis (PG), Hypogammalglobulinemia, IgG4-related sclerosing disease, Immune thrombocytopenic purpura (ITP), Inclusion body myositis (IBM), Lambert-Eaton syndrome, Leukocytoclastic vasculitis, Linear IgA disease (LAD), Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Primary biliary cirrhosis, Primary sclerosing cholangitis, Progesterone dermatitis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Thyroid eye disease (TED), Alagille Syndrome, Alcohol-Related Liver Disease, Autoimmune Hepatitis, Biliary Atresia, Cirrhosis, Lysosomal Acid Lipase Deficiency (LAL-D), Newborn Jaundice, Non-Alcoholic Fatty Liver Disease, Non-Alcoholic Steatohepatitis, Primary Biliary Cholangitis (PBC), Progressive Familial Intrahepatic Cholestasis (PFIC), Surgical scars, Hypertrophic scars, Acute Lymphoblastic Leukemia (ALL), Acute Myeloid Leukemia (AML), Adrenocortical Carcinoma, Kaposi Sarcoma (Soft Tissue Sarcoma), AIDS-Related Lymphoma (Lymphoma), Primary CNS Lymphoma (Lymphoma), Anal Cancer, Childhood astrocytomas, Atypical teratoid/rhabdoid tumor, Basal cell carcinoma of the skin, Bile duct cancer, Bladder cancer, Ewing Sarcoma, Osteosarcoma, Malignant fibrous histiocytoma, Brain tumor, Breast cancer, Bronchial tumor, Burkitt Lymphoma, Carcinoid Tumor (Gastrointestinal), Cardiac (Heart) Tumors, Childhood, Medulloblastoma, Germ Cell Tumor, Primary CNS Lymphoma, Cervical Cancer, Cholangiocarcinoma, Chordoma, Chronic Lymphocytic Leukemia (CLL), Chronic Myelogenous Leukemia (CML), Chronic Myeloproliferative Neoplasms, Colorectal Cancer, Craniopharyngioma, Cutaneous T-Cell Lymphoma, Ductal Carcinoma In Situ (DCIS), Endometrial Cancer (Uterine Cancer), Ependymoma, Esophageal Cancer, Esthesioneuroblastoma (Head and Neck Cancer), Retinoblastoma, Fallopian Tube Cancer, Gallbladder Cancer, Gastric (Stomach) Cancer, Gastrointestinal Carcinoid Tumor, Gastrointestinal Stromal Tumors (GIST), Germ Cell Tumors, Childhood Extracranial Germ Cell Tumors, Extragonadal Germ Cell Tumors, Ovarian Germ Cell Tumors, Testicular Cancer, Gestational Trophoblastic Disease, Hairy Cell Leukemia, Head and Neck Cancer, Heart Tumor, HepatocellularCancer, Histiocytosis, Langerhans Cell, Hodgkin Lymphoma, Hypopharyngeal Cancer (Head and Neck Cancer), Intraocular Melanoma, Islet Cell Tumors, Kidney (Renal Cell) Cancer, Langerhans Cell Histiocytosis, Laryngeal Cancer (Head and Neck Cancer), Lip and Oral Cavity Cancer, Lung Cancer (Non-Small Cell, Small Cell, Pleuropulmonary Blastoma, and Tracheobronchial Tumor), Male Breast Cancer, Melanoma, Melanoma, Intraocular (Eye), Meningioma, Merkel Cell Carcinoma (Skin Cancer), Mesothelioma, Malignant, Metastatic Squamous Neck Cancer with Occult Primary (Head and Neck Cancer), Midline Tract Carcinoma, Multiple Endocrine Neoplasia Syndromes, Multiple Myeloma/Plasma Cell Neoplasms, Mycosis Fungoides, Myelodysplastic Syndromes, Myelodysplastic/Myeloproliferative Neoplasms, Neuroblastoma, Non-Hodgkin Lymphoma, Non-Small Cell Lung Cancer, Oral Cancer, Lip and Oral Cavity Cancer and Oropharyngeal Cancer (Head and Neck Cancer), Osteosarcoma and Undifferentiated Pleomorphic Sarcoma of Bone Treatment, Ovarian Cancer, Pancreatic Cancer, Pancreatic Neuroendocrine Tumors, Papillomatosis, Paraganglioma, Parathyroid Cancer, Penile Cancer, Pharyngeal Cancer, Pheochromocytoma, Pituitary Tumor, Plasma Cell Neoplasm/Multiple Myeloma, Pleuropulmonary Blastoma, Pregnancy and Breast Cancer, Primary Central Nervous System (CNS) Lymphoma, Primary Peritoneal Cancer, Prostate Cancer, Rectal Cancer, Renal Cell (Kidney) Cancer, Retinoblastoma, Ewing Sarcoma (Bone Cancer), Soft Tissue Sarcoma, Sezary Syndrome, Skin Cancer, Small Cell Lung Cancer, Small Intestine Cancer, Soft Tissue Sarcoma, Squamous Cell Carcinoma of the Skin, Squamous Neck Cancer with Occult Primary, Metastatic (Head and Neck Cancer), Stomach (Gastric) Cancer, T-Cell Lymphoma, Cutaneous—see Lymphoma (Mycosis Fungoides and Sbzary Syndrome), Testicular Cancer, Nasopharyngeal Cancer, Oropharyngeal Cancer, Hypopharyngeal Cancer, Thymoma and Thymic Carcinoma, Thyroid Cancer, Tracheobronchial Tumors, Transitional Cell Cancer of the Renal Pelvis and Ureter, Urethral Cancer, Uterine Cancer, Endometrial, Uterine Sarcoma, Vaginal Cancer, Vulvar Cancer, and Wilms Tumor.

Embodiment 39. The method according to embodiment 38, wherein the disease is selected from the group consisting of brain tumor, gastric cancer, colorectal cancer, mesothelioma, non-small cell lung cancer, meningioma, head and neck cancer, and soft tissue sarcoma

Embodiment 40. The method according to embodiment 38 or 39, further comprising administering at least one additional chemotherapeutic agent.

Embodiment 41. The method according to embodiment 40, wherein the additional chemotherapeutic agent is selected from a B-RAF inhibitor, epidermal growth factor receptor (EGFR) inhibitor, MEK inhibitor, and immune check point inhibitor.

Embodiment 42. The method according to embodiment 41, wherein the B-RAF inhibitor is selected from the group consisting of vemurafenib, dabrafenib, and encorafenib.

Embodiment 43. The method according to embodiment 41, wherein the MEK inhibitor is selected from the group consisting of trametinib, cobimetinib, selumetinib, and binimetinib.

Embodiment 44. The method according to embodiment 41, wherein the EGFR inhibitor is selected from the group consisting of erlotinib, osimertinib, neratinib, gefitinib, cetuximab, panitumumab, dacomitinib, lapatinib, necitumumab, mobocertinib, and vandetanib.

Embodiment 45. The method according to embodiment 41, wherein the immune check point inhibitor is selected from the group consisting of a PD-1 inhibitor and PD-L1 inhibitor.

Embodiment 46. The method according to embodiment 45, wherein the PD-1 inhibitor is selected from the group consisting of pembrolizumab, nivolumab, and cemiplimab.

Embodiment 47. The method according to embodiment 45, wherein the PD-L1 inhibitor is selected from the group consisting of atezolizumab, avelumab, and durvalumab.

### Definitions

“Alkyl” refers to straight or branched chain hydrocarbyl including from 1 to about 20 carbon atoms. For instance, an alkyl can have from 1 to 10 carbon atoms or 1 to 6 carbon atoms. Exemplary alkyl includes straight chain alkyl groups such as methyl, ethyl, propyl, butyl, pentyl, hexyl, heptyl, octyl, nonyl, decyl, undecyl, dodecyl, and the like, and also includes branched chain isomers of straight chain alkyl groups, for example without limitation, —CH(CH₃)₂, —CH(CH₃)(CH₂CH₃), —CH(CH₂CH₃)₂, —C(CH₃)₃, —C(CH₂CH₃)₃, —CH₂CH(CH₃)₂, —CH₂CH(CH₃)(CH₂CH₃), —CH₂CH(CH₂CH₃)₂, —CH₂C(CH₃)₃, —CH₂C(CH₂CH₃)₃, —CH(CH ₃)CH(CH₃)(CH₂CH₃), —CH₂CH₂CH(CH₃)₂, —CH₂CH₂CH(CH₃)(CH₂CH₃), —CH₂CH₂CH(CH₂CH₃) 2, —CH₂CH₂C(CH₃)₃, —CH₂CH₂C(CH₂CH₃)₃, —CH(CH₃)CH₂CH(CH₃)₂, —CH(CH₃)CH(CH₃)CH(C H₃)₂, and the like. Thus, alkyl groups include primary alkyl groups, secondary alkyl groups, and tertiary alkyl groups. An alkyl group can be unsubstituted or optionally substituted with one or more substituents as described herein.

Each of the terms “halogen,” “halide,” and “halo” refers to —F or fluoro, —C₁ or chloro, —Br or bromo, or —I or iodo.

The term “alkenyl” refers to straight or branched chain hydrocarbyl groups including from 2 to about 20 carbon atoms having 1-3, 1-2, or at least one carbon to carbon double bond. An alkenyl group can be unsubstituted or optionally substituted with one or more substituents as described herein.

“Alkyne or “alkynyl” refers to a straight or branched chain unsaturated hydrocarbon having the indicated number of carbon atoms and at least one triple bond. Examples of a (C₂-C₅)alkynyl group include, but are not limited to, acetylene, propyne, 1-butyne, 2-butyne, 1-pentyne, 2-pentyne, 1-hexyne, 2-hexyne, 3-hexyne, 1-heptyne, 2-heptyne, 3-heptyne, 1-octyne, 2-octyne, 3-octyne and 4-octyne. An alkynyl group can be unsubstituted or optionally substituted with one or more substituents as described herein.

The term “alkoxy” or “alkoxyl” refers to an —O-alkyl group having the indicated number of carbon atoms. For example, a (C₁-C₆)-alkoxy group includes —O-methyl, —O-ethyl, —O-propyl, —O-isopropyl, —O-butyl, —O-sec-butyl, —O-tert-butyl, —O-pentyl, —O-isopentyl, —O— neopentyl, —O-hexyl, —O-isohexyl, and —O-neohexyl.

The term “cycloalkyl” refers to a saturated monocyclic, bicyclic, tricyclic, or polycyclic, 3- to 14-membered ring system, such as a C₃-C₈-cycloalkyl. The cycloalkyl may be attached via any atom. Representative examples of cycloalkyl include, but are not limited to cyclopropyl, cyclobutyl, cyclopentyl, and cyclohexyl. Polycyclic cycloalkyl includes rings that can be fused, bridged, and/or spiro-fused. A cycloalkyl group can be unsubstituted or optionally substituted with one or more substituents as described herein.

“Aryl” when used alone or as part of another term means a carbocyclic aromatic group whether or not fused having the number of carbon atoms designated or if no number is designated, up to 14 carbon atoms, such as a C₆-C₁₀-aryl or C₆-C₁₄-aryl. Examples of aryl groups include phenyl, naphthyl, biphenyl, phenanthrenyl, naphthacenyl, and the like (see e.g. Lang's Handbook of Chemistry (Dean, J. A., ed) 13^{th} ed. Table 7-2 [1985]). “Aryl” also contemplates an aryl ring that is part of a fused polycyclic system, such as aryl fused to cycloalkyl as defined herein. An exemplary aryl is phenyl. An aryl group can be unsubstituted or optionally substituted with one or more substituents as described herein.

The term “heteroatom” refers to N, O, and S. Compounds of the present disclosure that contain N or S atoms can be optionally oxidized to the corresponding N-oxide, sulfoxide, or sulfone compounds.

“Heteroaryl,” alone or in combination with any other moiety described herein, is a monocyclic aromatic ring structure containing 5 to 10, such as 5 or 6 ring atoms, or a bicyclic aromatic group having 8 to 10 atoms, containing one or more, such as 1-4, 1-3, or 1-2, heteroatoms independently selected from the group consisting of O, S, and N. Heteroaryl is also intended to include oxidized S or N, such as sulfinyl, sulfonyl and N-oxide of a tertiary ring nitrogen. A carbon or heteroatom is the point of attachment of the heteroaryl ring structure such that a stable compound is produced. Examples of heteroaryl groups include, but are not limited to, pyridinyl, pyridazinyl, pyrazinyl, quinaoxalyl, indolizinyl, benzo[b]thienyl, quinazolinyl, purinyl, indolyl, quinolinyl, pyrimidinyl, pyrrolyl, pyrazolyl, oxazolyl, thiazolyl, thienyl, isoxazolyl, oxathiadiazolyl, isothiazolyl, tetrazolyl, imidazolyl, triazolyl, furanyl, benzofuryl, and indolyl. A heteroaryl group can be unsubstituted or optionally substituted with one or more substituents as described herein.

“Heterocycloalkyl” is a saturated or partially unsaturated non-aromatic monocyclic, bicyclic, tricyclic or polycyclic ring system that has from 3 to 14, such as 3 to 6, atoms in which 1 to 3 carbon atoms in the ring are replaced by heteroatoms of O, S or N. Polycyclic heterocycloalkyl includes rings that can be fused, bridged, and/or spiro-fused. In addition, a heterocycloalkyl is optionally fused with aryl or heteroaryl of 5-6 ring members, and includes oxidized S or N, such as sulfinyl, sulfonyl and N-oxide of a tertiary ring nitrogen. The point of attachment of the heterocycloalkyl ring is at a carbon or heteroatom such that a stable ring is retained. Examples of heterocycloalkyl groups include without limitation morpholino, tetrahydrofuranyl, dihydropyridinyl, piperidinyl, pyrrolidinyl, piperazinyl, dihydrobenzofuryl, and dihydroindolyl. A heterocycloalkyl group can be unsubstituted or optionally substituted with one or more substituents as described herein.

The term “nitrile” or “cyano” can be used interchangeably and refers to a —CN group.

A “hydroxyl” or “hydroxy” refers to an —OH group.

Compounds described herein can exist in various isomeric forms, including configurational, geometric, and conformational isomers, including, for example, cis- or trans-conformations. The compounds may also exist in one or more tautomeric forms, including both single tautomers and mixtures of tautomers. The term “isomer” is intended to encompass all isomeric forms of a compound of this disclosure, including tautomeric forms of the compound. The compounds of the present disclosure may also exist in open-chain or cyclized forms. In some cases, one or more of the cyclized forms may result from the loss of water. The specific composition of the open-chain and cyclized forms may be dependent on how the compound is isolated, stored or administered. For example, the compound may exist primarily in an open-chained form under acidic conditions but cyclize under neutral conditions. All forms are included in the disclosure.

Some compounds described herein can have asymmetric centers and therefore exist in different enantiomeric and diastereomeric forms. A compound as described herein can be in the form of an optical isomer or a diastereomer. Accordingly, the disclosure encompasses compounds and their uses as described herein in the form of their optical isomers, diastereoisomers and mixtures thereof, including a racemic mixture. Optical isomers of the compounds of the disclosure can be obtained by known techniques such as asymmetric synthesis, chiral chromatography, simulated moving bed technology or via chemical separation of stereoisomers through the employment of optically active resolving agents.

Unless otherwise indicated, the term “stereoisomer” means one stereoisomer of a compound that is substantially free of other stereoisomers of that compound. Thus, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, for example greater than about 90% by weight of one stereoisomer of the compound and less than about 10% by weight of the other stereoisomers of the compound, or greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound, or greater than about 99% by weight of one stereoisomer of the compound and less than about 1% by weight of the other stereoisomers of the compound. The stereoisomer as described above can be viewed as composition comprising two stereoisomers that are present in their respective weight percentages described herein.

If there is a discrepancy between a depicted structure and a name given to that structure, then the depicted structure controls. Additionally, if the stereochemistry of a structure or a portion of a structure is not indicated with, for example, bold or dashed lines, the structure or portion of the structure is to be interpreted as encompassing all stereoisomers of it. In some cases, however, where more than one chiral center exists, the structures and names may be represented as single enantiomers to help describe the relative stereochemistry. Those skilled in the art of organic synthesis will know if the compounds are prepared as single enantiomers from the methods used to prepare them.

As used herein, and unless otherwise specified to the contrary, the term “compound” is inclusive in that it encompasses a compound or a pharmaceutically acceptable salt, stereoisomer, and/or tautomer thereof. Thus, for instance, a compound of Formula I includes a pharmaceutically acceptable salt of a tautomer of the compound.

In this disclosure, a “pharmaceutically acceptable salt” is a pharmaceutically acceptable, organic or inorganic acid or base salt of a compound described herein. Representative pharmaceutically acceptable salts include, e.g., alkali metal salts, alkali earth salts, ammonium salts, water-soluble and water-insoluble salts, such as the acetate, amsonate (4,4-diaminostilbene-2,2-disulfonate), benzenesulfonate, benzoate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts. A pharmaceutically acceptable salt can have more than one charged atom in its structure. In this instance the pharmaceutically acceptable salt can have multiple counterions. Thus, a pharmaceutically acceptable salt can have one or more charged atoms and/or one or more counterions.

The terms “treat”, “treating” and “treatment” refer to the amelioration or eradication of a disease or symptoms associated with a disease. In various embodiments, the terms refer to minimizing or slowing the spread, progression, or worsening of the disease resulting from the administration of one or more prophylactic or therapeutic compounds described herein to a patient with such a disease.

The terms “prevent,” “preventing,” and “prevention” refer to the prevention of the onset, recurrence, or spread of the disease in a patient resulting from the administration of a compound described herein.

The term “effective amount” refers to an amount of a compound as described herein or other active ingredient sufficient to provide a therapeutic or prophylactic benefit in the treatment or prevention of a disease or to delay or minimize symptoms associated with a disease. Further, a therapeutically effective amount with respect to a compound as described herein means that amount of therapeutic agent alone, or in combination with other therapies, that provides a therapeutic benefit in the treatment or prevention of a disease. Used in connection with a compound as described herein, the term can encompass an amount that improves overall therapy, reduces or avoids symptoms or causes of disease, or enhances the therapeutic efficacy of or is synergistic with another therapeutic agent.

A “patient” or subject” includes an animal, such as a human, cow, horse, sheep, lamb, pig, chicken, turkey, quail, cat, dog, mouse, rat, rabbit or guinea pig. In accordance with some embodiments, the animal is a mammal such as a non-primate and a primate (e.g., monkey and human). In one embodiment, a patient is a human, such as a human infant, child, adolescent or adult. In the present disclosure, the terms “patient” and “subject” are used interchangeably.

### COMPOUNDS

In various embodiments, the present disclosure provides a compound of formula (I), a tautomer, or a pharmaceutically acceptable salt thereof:

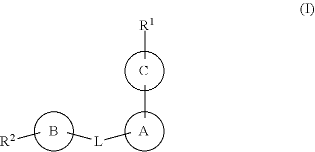

Ring A is a 6-membered aryl or 6-membered heteroaryl having 1, 2, or 3 nitrogen ring members.
Ring B is a C₆-C₁₂-aryl, C₄-C₇-cycloalkyl, or 6-membered heteroaryl having 1 or 2 nitrogen ring members.
Ring C is a 4- to 10-membered heterocycle containing 1 or 2 N ring members and that is optionally fused and optionally has 1 to 3 unsaturated bonds.

L is selected from the group consisting of —NH—, —O—, —CH₂—, —S—, —C(O)—, —CHOH—, and a bond.

In various embodiments, R¹ is bound to a carbon ring member, and R¹ is selected from the group consisting of CN, halo, —[CH₂]_{0,1}NHC(O)(C₁-C₆-alkyl), —[CH₂]_{0,1}NHC(O)(C₂-C₆-alkenyl), and —[CH₂]_{0,1}NHC(O)(C₂-C₆-alkynyl), —[CH₂]_{0,1}NHC(O)(C₃-C₁₀-cycloalkyl), —[CH₂]_{0,1}NHC(O)(3- to 6-membered heterocycloalkyl (wherein 1-4 ring members are independently selected from N, O, and S)), —[CH₂]O,1NHSO₂(C₁-C₆-alkyl), —[CH₂]_{0,1}NHSO₂(C₂-C₆-alkenyl), and —[CH₂]_{0,1}NHSO₂(C₂-C₆-alkynyl), —[CH₂]_{0,1}NHSO₂(C₃-C₁₀-cycloalkyl), and —[CH₂]_{0,1}NHSO₂(3- to 6-membered heterocycloalkyl (wherein 1-4 ring members are independently selected from N, O, and S)).

In other embodiments, R¹ is bound to a nitrogen ring member, and R¹ is selected from the group consisting of CN, —C(O)(C₁-C₆-alkyl), —C(O)(C₂-C₆-alkenyl), —C(O)(C₂-C₆-alkynyl), —C(O)(C₃-C₁₀-cycloalkyl), —C(O)(3- to 6-membered heterocycloalkyl (wherein 1-4 ring members are independently selected from N, O, and S)), —C₁-C₆-alkyl(C₃-C₁₀-cycloalkyl), —SO₂(C₁-C₆-alkyl), —SO₂(C₂-C₆-alkenyl), —SO₂(C₂-C₆-alkynyl), —SO₂(C₃-C₁₀-cycloalkyl), and —SO₂(3- to 6-membered heterocycloalkyl (wherein 1-4 ring members are independently selected from N, O, and S)).

R² is selected from the group consisting of halo, C₁-C₆-haloalkyl, C₁-C₆-haloalkoxy, and —SF₅.

Ring A, Ring B, and Ring C are independently and optionally substituted with 1 to 3 three substituents independently selected from halo and C₁-C₆-alkyl.

In Formula (I), any alkyl, alkenyl, alkynyl, cycloalkyl, and heterocycloalkyl is optionally substituted with one to three substituents independently selected from the group consisting of halo, OH, CN, C₁-C₆-alkoxy, C₁-C₆-alkyl, C₁-C₆-haloalkyl, C₁-C₆-hydroxyalkyl, (C₀-C₆-alkyl)NRR′, —(C₀-C₆-alkyl)-OC(O)R, —(C₀-C₆-alkyl)-C(O)OR, C₃-C₁₀-cycloalkyl, 3- to 6-membered heterocycloalkyl (wherein 1-4 ring members are independently selected from N, 0, and S), —(C₁-C₆-alkyl)(3- to 6-membered heterocycloalkyl (wherein 1-4 ring members are independently selected from N, O, and S).

R and R′ are independently selected from H, C₁-C₆-alkyl, C₂-C₆-alkenyl, and C₂-C₆-alkynyl.

In some embodiments, Ring A is 6-membered heteroaryl having 2 or 3 nitrogen ring members. Illustrative examples of Ring A are selected from the group consisting of optionally substituted:

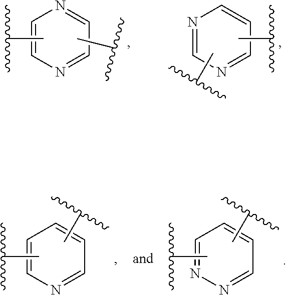

Thus, in one embodiment, Ring A is optionally substituted

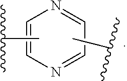

In another embodiment, Ring A is optionally substituted

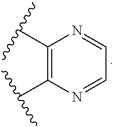 [SMILES: *c1nccnc1*]

In additional embodiments, optionally in combination with any other embodiment herein described, Ring B is optionally substituted C₆-C₁₂-aryl. In an illustrative embodiment, Ring B is optionally substituted phenyl.

In other embodiments, Ring B is an optionally substituted 6-membered heteroaryl having 1 or 2 nitrogen ring members. Examples of Ring B are selected from the group of optionally substituted:

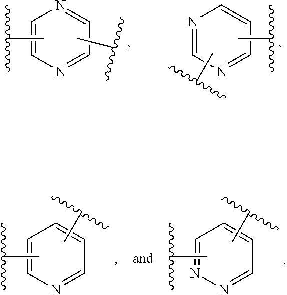

The present disclosure also provides, in some embodiments, Formula (I) compounds wherein Ring C is an optionally substituted 4- to 8-membered heterocycle. In one embodiment, Ring C contains 1 N ring member. In another embodiment, Ring C contains 2 N ring members. Examples of Ring C include those selected from the group consisting of optionally substituted:

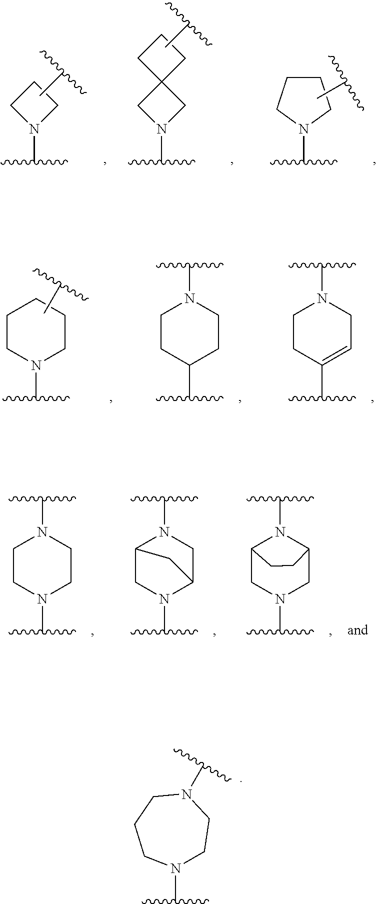

In illustrative embodiments, Ring C is optionally substituted

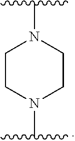 [SMILES: *N1CCN(*)CC1]

In accordance with various embodiments, optionally in combination with any other embodiment described herein, L is —NH—.

Further embodiments of the present disclosure provide Formula (I) compounds wherein R² is C₁-C₆-haloalkyl. In an illustrative embodiment, R² is —CF₃.

In some embodiments, R¹ is bound to a nitrogen ring member. For example, R¹ can have the formula:

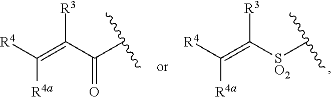

wherein R³, R⁴, and R^{4a} are individually selected from the group consisting of H, halo, CN, —C(O)OR, and optionally substituted C₁-C₆-alkyl. In an illustrative embodiment, R¹ has the formula

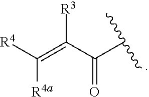

In these embodiments:
R³ is selected from the group consisting of H, F, C₁, CH₂F, CF₃, CN, CH₂OH, CH₂OC(O)R, and CH₂NRR′; and
R⁴ and R^{4a} are independently selected from the group consisting of H, CH₂F, CHF₂, CH₂OH, CH₂NRR′, and CH₂-(3- to 6-membered heterocycloalkyl (wherein 1-4 ring members are independently selected from N, O, and S), wherein R and R′ are as defined herein.

In other embodiments, wherein R¹ is bound to a nitrogen ring member, R¹ has the formula:

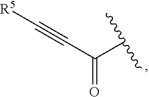

wherein R⁵ is selected from the group consisting of H, C₁-C₆-alkyl, C₁-C₆-haloalkyl, and C₁-C₆-hydroxyalkyl.

In still additional embodiments, wherein R¹ is bound to a nitrogen ring member, R¹ has the formula:

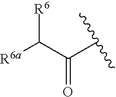

wherein R⁶ and R^{6a} are independently selected from the group consisting of H, halo, CN, and C₁-C₆-alkyl. In some embodiments, at least one of R⁶ and R^{6a} is halo. In other embodiments, each of R⁶ and R^{6a} is halo. In still other embodiments, at least one of R⁶ and R^{6a} is CN. All of these combinations are contemplated.

In further embodiments, wherein R¹ is bound to a nitrogen ring member, R¹ has the formula:

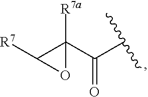

wherein R⁷ and R^{7a} are independently selected from the group consisting of H, optionally substituted alkyl, and —C(O)OR, wherein R is as defined herein.

The present disclosure provides in various embodiments a Formula (I) compound wherein R¹ is selected from the group consisting of:

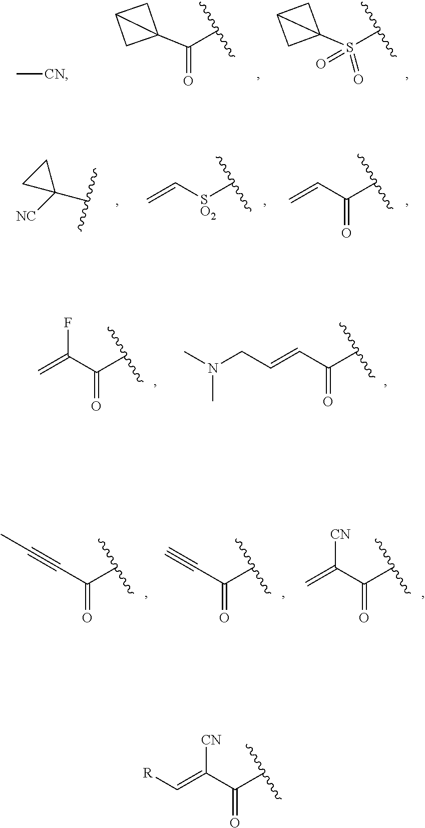

(wherein R is substituted alkyl),

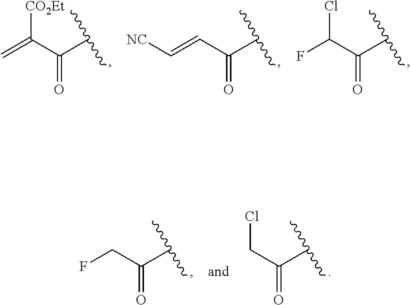

A specific example of R¹, in accordance with some embodiments, and optionally in combination with any other embodiment described herein, is the following formula:

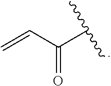 [SMILES: *C(=O)C=C]

Some compounds of Formula (1) as described herein, per various embodiments, also conform to Formula (IA):

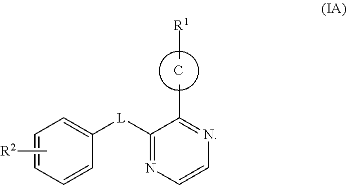

In Formula (IA), per some embodiments, L is —NH—. In additional embodiments, R² is —CF₃ or —SF₅. R² can be bound to any position on the phenyl ring in Formula (IA). In illustrative embodiments, R² is in the para position with respect to L, i.e.:

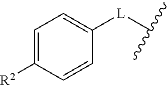

In further embodiments, concerning Formula (IA), Ring C is a 4- to 6-membered heterocycle containing 2 N ring members. An example of Ring C, per an illustrative embodiment, is:

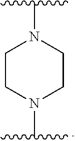 [SMILES: *N1CCN(*)CC1]

The present disclosure also provides in various embodiments a compound, or a tautomer or a pharmaceutically acceptable salt thereof, as set forth in Table 1.

TABLE 1
Exemplary Compounds

| | | | H226 | | | |
|---|---|---|---|---|---|---|
| | | H226 | IC₅₀ | | IM | |
| | | IC₅₀ | (uM) | MCF7 | R32 | |
| Cpd | | (uM) | (In | IC₅₀ | IC₅₀ | |
| # | Structure | (Wuxi) | house) | (uM) | (uM) | ¹H NMR |
| 1 | 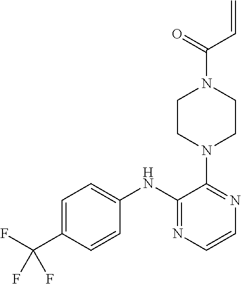 [SMILES: C=CC(=O)N1CCN(c2nccnc2Nc2ccc(C(F)(F)F)cc2)CC1] | 0.0143 | 0.0166 | 0.0018 | >10 | ¹H NMR (400 MHz, DMSO) δ 8.61 (s, 1H), 7.97-7.77 (m, 4H), 7.63 (d, J = 8.6 Hz, 2H), 6.84 (dd, J = 16.7, 10.5 Hz, 1H), 6.14 (dd, J = 16.7, 2.4 Hz, 1H), 5.71 (dd, J = 10.4, 2.4 Hz, 1H), 3.78 (d, J = 12.8 Hz, 4H), 3.14 (s, 4H) |
| 2 | 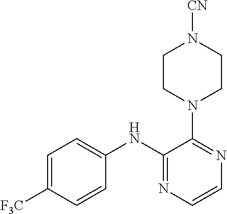 [SMILES: N#CN1CCN(c2nccnc2Nc2ccc(C(F)(F)F)cc2)CC1] | — | 0.252 | 0.468 | >20 | ¹H NMR (400 MHz, DMSO) δ 8.55 (s, 1H), 7.93-7.80 (m, 4H), 7.63 (d, J = 8.6 Hz, 2H), 3.49-3.40 (m, 4H), 3.24-3.13 (m, 4H). |
| 3 | 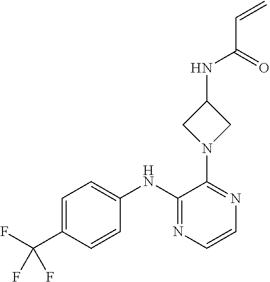 [SMILES: C=CC(=O)NC1CN(c2nccnc2Nc2ccc(C(F)(F)F)cc2)C1] | — | 8.24 | 3.76 | 10 | ¹H NMR (400 MHz, DMSO) δ 10.86-10.30 (m, 1H), 8.59-8.31 (m, 1H), 8.17-7.82 (m, 2H), 7.77-7.59 (m, 2H), 7.59-7.33 (m, 2H), 6.31-6.07 (m, 2H), 5.71-5.59 (m, 1H), 4.67 (s, 1H), 4.48-4.13 (m, 2H), 3.85-3.50 (m, 3H). |
| 4 | 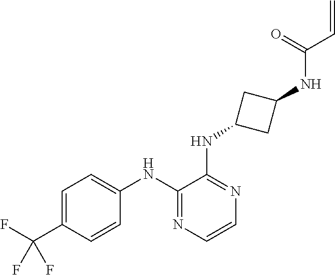 [SMILES: C=CC(=O)N[C@H]1C[C@H](Nc2nccnc2Nc2ccc(C(F)(F)F)cc2)C1] | — | 23.94 | 0.008 | — | ¹H NMR (400 MHz, DMSO) δ 8.73-8.54 (m, 2H), 7.88 (d, J = 8.5 Hz, 2H), 7.65 (d, J = 8.6 Hz, 2H), 7.56 (d, J = 3.0 Hz, 1H), 7.43 (d, J = 3.0 Hz, 1H), 7.00 (d, J = 5.7 Hz, 1H), 6.24 (dd, J = 17.1, 10.1 Hz, 1H), 6.10 (dd, J = 17.0, 2.3 Hz, 1H), 5.61 (dd, J = 10.0, 2.3 Hz, 1H), 4.51-4.39 (m, 2H), 2.34 (ddt, J = 9.6, 5.5, 2.0 Hz, 4H). |
| 5 | 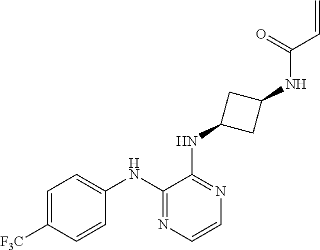 [SMILES: C=CC(=O)N[C@H]1C[C@@H](Nc2nccnc2Nc2ccc(C(F)(F)F)cc2)C1] | — | 13.66 | 0.408 | >20 | ¹H NMR (400 MHz, DMSO) δ 8.64 (s, 1H), 8.42 (d, J = 7.2 Hz, 1H), 7.88 (d, J = 8.5 Hz, 2H), 7.65 (d, J = 8.6 Hz, 2H), 7.57 (d, J = 3.0 Hz, 1H), 7.42 (d, J = 3.0 Hz, 1H), 6.93 (s, 1H), 6.27-6.05 (m, 2H), 5.60 (dd, J = 9.9, 2.5 Hz, IH), 4.06 (dd, J = 16.6, 8.7 Hz, 2H), 2.84-2.72 (m, 2H), 1.98-1.84 (m, 2H). |
| 6 | 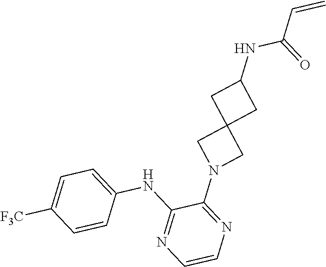 [SMILES: C=CC(=O)NC1CC2(C1)CN(c1nccnc1Nc1ccc(C(F)(F)F)cc1)C2] | — | >10 | >20 | >20 | ¹H NMR (400 MHz, DMSO) δ 8.45 (s, 1H), 8.36 (d, J = 7.4 Hz, 1H), 7.77-7.68 (m, 3H), 7.66-7.53 (m, 3H), 6.21-6.02 (m, 2H), 5.59 (dd, J = 9.7, 2.6 Hz, 1H), 4.16 (d, J = 17.6 Hz, 3H), 4.03 (s, 2H), 2.56-2.54 (m, 2H), 2.14 (td, J = 8.8, 2.9 Hz, 2H). |
| 7 | 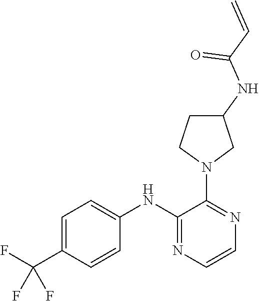 [SMILES: C=CC(=O)NC1CCN(c2nccnc2Nc2ccc(C(F)(F)F)cc2)C1] | — | 0.195 | 0.0286 | >10 | ¹H NMR (400 MHz, DMSO) δ 8.61 (s, 1H), 8.33 (d, J = 6.5 Hz, 1H), 7.76 (d, J = 2.7 Hz, 1H), 7.69-7.49 (m, 5H), 6.21 (dd, J = 17.1, 10.0 Hz, 1H), 6.08 (dd, J = 17.1, 2.3 Hz, 1H), 5.58 (dd, J = 10.0, 2.3 Hz, 1H), 4.35 (q, J = 6.1 Hz, 1H), 3.74 (dd, J = 10.7, 6.5 Hz, 1H), 3.56 (d, J = 6.9 Hz, 2H), 3.31 (dd, J = 10.7, 5.3 Hz, 1H), 2.16 (dd, J = 12.6, 6.4 Hz, 1H), 1.86 (dd, J = 12.5, 6.2 Hz, 1H). |
| 8 | 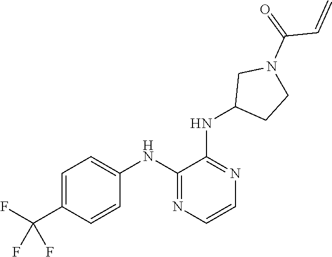 [SMILES: C=CC(=O)N1CCC(Nc2nccnc2Nc2ccc(C(F)(F)F)cc2)C1] | — | 8.48 | 0.0493 | >5 | ¹H NMR (400 MHz, DMSO) δ 8.60 (d, J = 4.1 Hz, 1H), 7.85 (d, J = 8.5 Hz, 2H), 7.69-7.58 (m, 3H), 7.47 (t, J = 2.8 Hz, 1H), 6.77 (dd, J = 13.2, 5.5 Hz, 1H), 6.70-6.51 (m, 1H), 6.16 (ddd, J = 16.8, 8.4, 2.4 Hz, 1H), 5.69 (ddd, J = 12.7, 10.3, 2.4 Hz, 1H), 4.63-4.43 (m, 1H), 4.07-3.70 (m, 2H), 3.63-3.41 (m, 2H), 2.37-1.94 (m, 2H). |
| 9 | 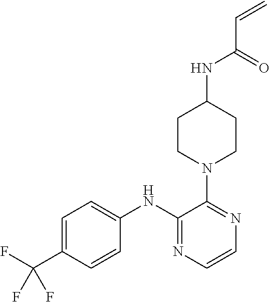 [SMILES: C=CC(=O)NC1CCN(c2nccnc2Nc2ccc(C(F)(F)F)cc2)CC1] | — | >10 | 0.0296 | 10 | ¹H NMR (400 MHz, DMSO) δ 8.52 (s, 1H), 8.12 (d, J = 7.4 Hz, 1H), 7.88 (d, J = 8.5 Hz, 2H), 7.84-7.76 (m, 2H), 7.62 (d, J = 8.6 Hz, 2H), 6.26 (dd, J = 17.1, 10.1 Hz, 1H), 6.09 (dd, J = 17.1, 2.3 Hz, 1H), 5.57 (dd, J = 10.1, 2.3 Hz, 1H), 3.84 (d, J = 11.2 Hz, 1H), 3.51 (d, J = 13.0 Hz, 2H), 3.00-2.84 (m, 2H), 1.88 (d, J = 12.3 Hz, 2H), 1.77-1.65 (m, 2H). |
| 10 | 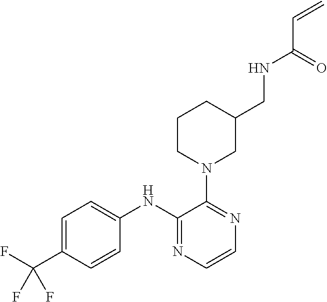 [SMILES: C=CC(=O)NCC1CCCN(c2nccnc2Nc2ccc(C(F)(F)F)cc2)C1] | — | >14 | 11.35 | 10 | ¹H NMR (400 MHz, DMSO) δ 8.33 (s, 1H), 8.18 (1, J = 5.9 Hz, 1H), 7.88-7.76 (m, 4H), 7.59 (d, J = 8.7 Hz, 2H), 6.13 (dd, J = 17.1, 10.0 Hz, 1H), 6.00 (dd, J = 17.1, 2.3 Hz, 1H), 5.49 (dd, J = 10.0, 2.3 Hz, 1H), 3.77-3.53 (m, 4H), 3.18-2.97 (m, 2H), 1.99 (s, 1H), 1.83-1.65 (m, 3H), 1.24-1.01 (m, 1H). |
| 11 | 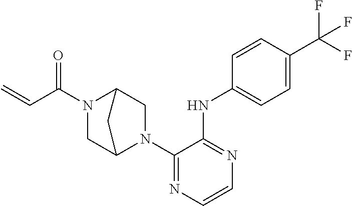 [SMILES: C=CC(=O)N1CC2CC1CN2c1nccnc1Nc1ccc(C(F)(F)F)cc1] | — | 0.483 | 0.0435 | 1.41 | ¹H NMR (400 MHz, DMSO) δ 8.46 (s, 1H), 7.75 (d, J = 2.8 Hz, 1H), 7.67 (dd, J = 3.8, 2.8 Hz, 1H), 7.61- 7.53 (m, 4H), 6.75- 6.31 (m, 1H), 6.10 (ddd, J = 16.8, 9.3, 2.4 Hz, 1H), 5.63 (ddd, J = 14.3, 10.2, 2.4 Hz, 1H), 4.91-4.67 (m, 2H), 4.04-3.61 (m, 4H), 1.98-1.85 (m, 2H). |
| 12 | 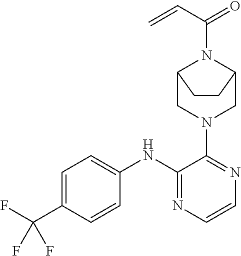 [SMILES: C=CC(=O)N1C2CCC1CN(c1nccnc1Nc1ccc(C(F)(F)F)cc1)C2] | — | 1.16 | 0.491 | 10 | ¹H NMR (400 MHz, DMSO) δ 8.33 (s, 1H), 7.87 (q, J = 2.7 Hz, 2H), 7.72-7.54 (m, 4H), 6.74 (dd, J = 16.7, 10.4 Hz, 1H), 6.19 (dd, J = 16.7, 2.4 Hz, 1H), 5.71 (dd, J = 10.3, 2.4 Hz, 1H), 4.61 (d, J = 7.6 Hz, 2H), 3.45 (d, J = 12.0 Hz, 2H), 2.97 (dd, J = 17.1, 11.9 Hz, 2H), 2.03-1.68 (m, 4H). |
| 13 | 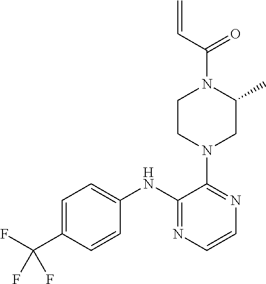 [SMILES: C=CC(=O)N1CCN(c2nccnc2Nc2ccc(C(F)(F)F)cc2)C[C@H]1C] | — | 0.0159 | 0.003485 | >10 | ¹H NMR (400 MHz, DMSO) δ 8.34 (s, 1H), 7.89 (d, J = 2.8 Hz, 1H), 7.87-7.79 (m, 3H), 7.65 (d, J = 8.6 Hz, 2H), 6.81 (dd, J = 16.7, 10.5 Hz, 1H), 6.14 (dd, J = 16.6, 2.4 Hz, 1H), 5.70 (dd, J = 10.5, 2.4 Hz, 1H), 3.56 (d, J = 12.3 Hz, 1H), 3.43-3.30 (m, 4H), 2.93 (s, 1H), 2.68 (d, J = 9.5 Hz, 1H), 1.42- 1.22 (m, 3H). |
| 14 | 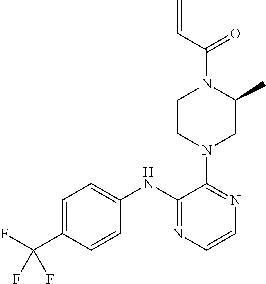 [SMILES: C=CC(=O)N1CCN(c2nccnc2Nc2ccc(C(F)(F)F)cc2)C[C@@H]1C] | — | 0.0246 | 0.002941 | >10 | ¹H NMR (400 MHz, DMSO) δ 8.36 (s, 1H), 7.95-7.81 (m, 4H), 7.66 (d, J = 8.6 Hz, 2H), 6.83 (dd, J = 16.6, 10.5 Hz, 1H), 6.15 (dd, J = 16.7, 2.5 Hz, 1H), 5.71 (dd, J = 10.5, 2.4 Hz, 1H), 3.58 (d, J = 12.5 Hz, 1H), 3.35 (s, 4H), 2.94 (s, IH), 2.81- 2.62 (m, 1H), 1.36 (d, J = 6.7 Hz, 3H). |
| 15 | 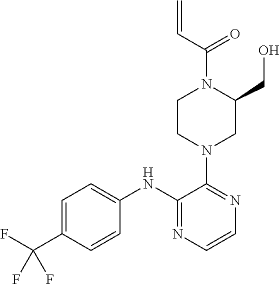 [SMILES: C=CC(=O)N1CCN(c2nccnc2Nc2ccc(C(F)(F)F)cc2)C[C@@H]1CO] | — | >20 | 5 | 10 | ¹H NMR (400 MHz, DMSO) δ 8.99 (s, 1H), 8.08 (d, J = 8.5 Hz, 2H), 7.79 (d, J = 3.0 Hz, 1H), 7.68 (d, J = 8.5 Hz, 2H), 7.61 (d, J = 3.1 Hz, 1H), 6.85 (td, J = 16.0. 10.4 Hz, 1H), 6.12 (dd, J = 16.6, 2.4 Hz, 1H), 5.68 (t, J = 8.8 Hz, 1H), 4.48-3.87 (m, 4H), 3.26-2.94 (m, 4H), 2.90-2.57 (m, 2H). |
| 16 | 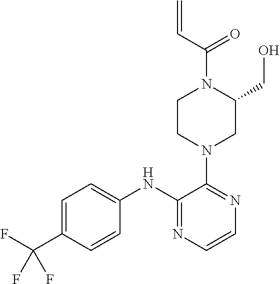 [SMILES: C=CC(=O)N1CCN(c2nccnc2Nc2ccc(C(F)(F)F)cc2)C[C@H]1CO] | — | >20 | 2.5 | 5 | ¹H NMR (400 MHz, DMSO) δ 8.99 (s, 1H), 8.08 (d, J = 8.5 Hz, 2H), 7.79 (d, J = 3.0 Hz, 1H), 7.68 (d, J = 8.5 Hz, 2H), 7.61 (d, J = 3.0 Hz, 1H), 6.85 (td, J = 16.0, 10.4 Hz, 1H), 6.12 (dd, J = 16.7, 2.4 Hz, 1H), 5.68 (td, J = 9.9, 2.5 Hz, 1H), 4.48- 3.89 (m, 4H), 3.21- 2.94 (m, 4H), 2.90- 2.59 (m, 2H). |
| 17 | 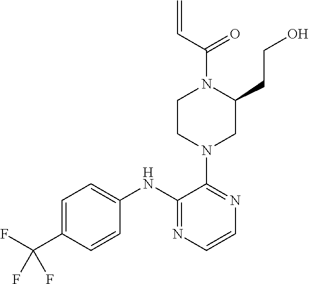 [SMILES: C=CC(=O)N1CCN(c2nccnc2Nc2ccc(C(F)(F)F)cc2)C[C@@H]1CCO] | — | 3.3 | >20 | | ¹H NMR (400 MHz, DMSO) δ 8.97 (s, 1H), 8.10 (d, J = 8.4 Hz, 2H), 7.76 (d, J = 3.0 Hz, 1H), 7.71-7.51 (m, 3H), 6.80 (dd, J = 16.6, 10.4 Hz, 1H), 6.18-5.98 (m, 1H), 5.73-5.53 (m, 1H), 4.52 (dq, J = 11.0, 5.7 Hz, 2H), 4.33-4.08 (m, 1H), 4.06-3.80 (m, 1H), 3.19-2.78 (m, 2H), 2.78-2.54 (m, 3H), 2.03-1.74 (m, 2H). |
| 18 | 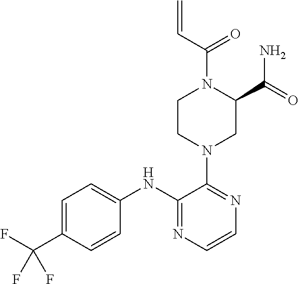 [SMILES: C=CC(=O)N1CCN(c2nccnc2Nc2ccc(C(F)(F)F)cc2)C[C@@H]1C(N)=O] | | 0.701 | 0.134 | | ¹H NMR (400 MHz, DMSO) δ 9.60 (d, J = 8.9 Hz, 1H), 8.23 (d, J = 8.7 Hz, 2H), 8.12- 7.89 (m, 2H), 7.84- 7.59 (m, 3H), 6.92 (dd, J = 16.8, 10.6 Hz, 1H), 6.21 (dd, J = 16.8, 2.2 Hz, 1H), 5.84-5.72 (m, 1H), 5.39-5.00 (m, 1H), 4.30-3.91 (m, 2H), 3.07-2.86 (m, 2H), 2.42-2.26 (m, 2H). |
| 19 | 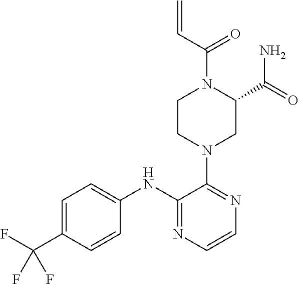 [SMILES: C=CC(=O)N1CCN(c2nccnc2Nc2ccc(C(F)(F)F)cc2)C[C@H]1C(N)=O] | — | >20 | >20 | — | ¹H NMR (400 MHz, DMSO) δ 9.60 (d, J = 9.1 Hz, 1H), 8.23 (d, J = 8.6 Hz, 2H), 8.10- 7.89 (m, 2H), 7.86- 7.70 (m, 2H), 7.68 (d, J = 8.6 Hz, 2H), 6.97- 6.79 (m, 1H), 6.21 (dd, J = 16.8, 2.3 Hz, 1H), 5.83-5.71 (m, 1H), 5.38-5.00 (m, 1H), 4.38-4.11 (m, 2H), 3.10-2.87 (m, 2H), 2.49-2.30 (m, 2H). |
| 20 | 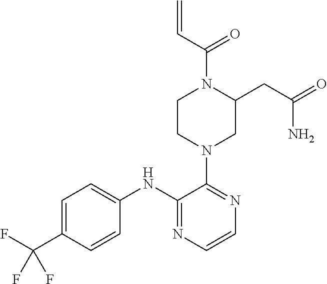 [SMILES: C=CC(=O)N1CCN(c2nccnc2Nc2ccc(C(F)(F)F)cc2)CC1CC(N)=O] | — | 2.73 | >20 | — | ¹H NMR (400 MHz, DMSO) δ 8.71-8.33 (m, 1H), 7.97-7.80 (m, 4H), 7.66 (d, J = 8.4 Hz, 2H), 7.47 (s, 1H), 7.09-6.73 (m, 2H), 6.14 (d, J = 16.6 Hz, 1H), 5.72 (d, J = 10.4 Hz, 1H), 5.00- 4.53 (m, 1H), 4.47- 3.87 (m, 1H), 3.75- 3.46 (m, 3H), 3.01- 2.66 (m, 4H). |
| 21 | 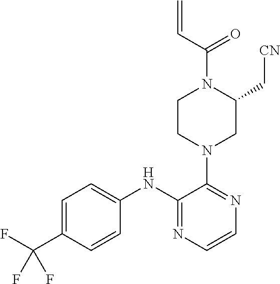 [SMILES: C=CC(=O)N1CCN(c2nccnc2Nc2ccc(C(F)(F)F)cc2)C[C@H]1CC#N] | — | 0.32 | 0.043 | >10 | ¹H NMR (400 MHz, DMSO) δ 8.41 (s, 1H), 8.01-7.79 (m, 4H), 7.69 (d, J = 8.5 Hz, 2H), 6.25-6.13 (m, 1H), 5.78 (d, J = 10.4 Hz, 1H), 5.10 (s, 1H), 3.84-3.49 (m, 4H), 3.01 (s, 3H). |
| 22 | 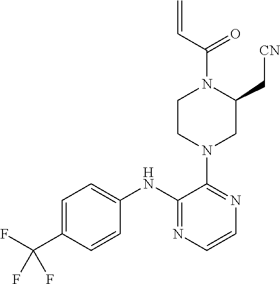 [SMILES: C=CC(=O)N1CCN(c2nccnc2Nc2ccc(C(F)(F)F)cc2)C[C@@H]1CC#N] | — | 0.27 | 0.0259 | 15 | ¹H NMR (400 MHz, CDCl₃) δ 8.07-8.02 (m, 2H), 7.98 (d, J = 2.2 Hz, 1H), 7.56-7.44 (m, 3H), 6.55 (d, J = 2.3 Hz, 1H), 6.19 (s, 1H), 4.38 (d, J = 12.3 Hz, 1H), 4.26 (dd, J = 12.3, 4.3 Hz, 1H), 4.16 (dp, .J = 4.2, 2.0 Hz, 1H), 4.11-3.99 (m, 2H), 3.42 (s, 3H), 2.28 (ddp, J = 5.5, 3.7, 1.9 Hz, 1H), 2.21-2.08 (m, 1H). |
| 23 | 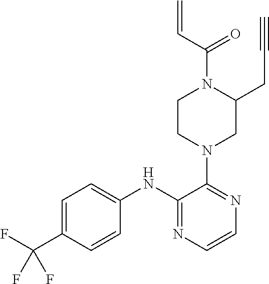 [SMILES: C#CCC1CN(c2nccnc2Nc2ccc(C(F)(F)F)cc2)CCN1C(=O)C=C] | 0.151 | 0.651 | 0.0003 | — | ¹H NMR (400 MHz, DMSO) δ 8.42 (s, 1H), 7.99-7.77 (m, 4H), 7.67 (d, J = 8.5 Hz, 2H), 6.88 (d, J = 12.5 Hz, 1H), 6.15 (d, J = 16.3 Hz, 1H), 5.73 (dd, J = 10.4, 2.4 Hz, 1H), 4.84-4.47 (m, 1H), 4.42-3.92 (m, 1H), 3.78-3.40 (m, 3H), 3.23-2.58 (m, 5H). |
| 24 | 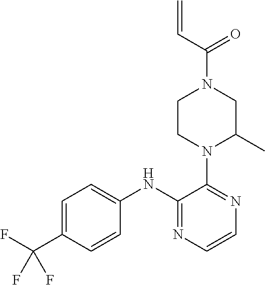 [SMILES: C=CC(=O)N1CCN(c2nccnc2Nc2ccc(C(F)(F)F)cc2)C(C)C1] | — | 0.0199 | 0.041 | 2.5 | ¹H NMR (400 MHz, DMSO) δ 8.69-8.61 (m, 1H), 7.97 (d, J = 9.4 Hz, 3H), 7.89 (d, J = 2.8 Hz, 1H), 7.66 (d, J = 8.5 Hz, 2H), 6.87 (dd, J = 16.7, 10.4 Hz, 1H), 6.17 (d, J = 17.1 Hz, 1H), 5.73 (dd, J = 10.4, 2.4 Hz, 1H), 4.06- 3.68 (m, 5H), 3.22 (d, J = 9.6 Hz, 2H), 2.86 (ddd, J = 11.8, 6.9, 3.4 Hz, 2H), 0.92 (d, J = 6.0 Hz, 3H). |
| 25 | 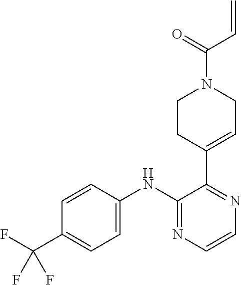 [SMILES: C=CC(=O)N1CC=C(c2nccnc2Nc2ccc(C(F)(F)F)cc2)CC1] | — | 0.006 | 0.0021 | 15.5 | ¹H NMR (400 MHz, DMSO): δ 8.74 (s, 1H), 8.11 (s, 2H), 7.81 (d, J = 11.6 Hz, 2H), 7.61 (d, J = 12.0 Hz, 2H), 6.95-6.86 (m, 1H), 6.33-6.13 (m, 2H), 5.74-5.70 (m, 1H), 4.29-4.22 (m, 2H), 3.80-3.78 (m, 2H), 2.61-2.58 (m, 2H). |
| 26 | 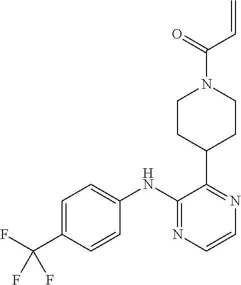 [SMILES: C=CC(=O)N1CCC(c2nccnc2Nc2ccc(C(F)(F)F)cc2)CC1] | 0.020 | 0.009 | 0.002 | 10 | ¹H NMR (400 MHz, DMSO) δ 8.82 (s, 1H), 8.07 (s, 2H), 7.85 (d, J = 8.5 Hz, 2H), 7.65 (d, J = 8.6 Hz, 2H), 6.87 (dd, J = 16.7, 10.5 Hz, 1H), 6.13 (dd, J = 16.7, 2.5 Hz, 1H), 5.69 (dd, J = 10.4, 2.5 Hz, 1H), 4.59 (d, J = 13.1 Hz, 1H), 4.21 (d, J = 13.5 Hz, 1H), 3.53 (t, J = 11.4 Hz, 1H), 3.28-3.13 (m, 1H), 2.83 (t, J = 12.8 Hz, 1H), 1.88 (d, J = 13.0 Hz, 2H), 1.78- 1.49 (m, 2H). |
| 27 | 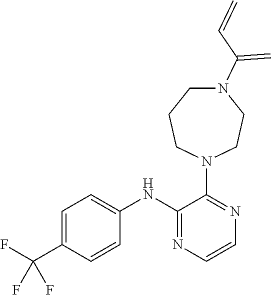 [SMILES: C=CC(=C)N1CCCN(c2nccnc2Nc2ccc(C(F)(F)F)cc2)CC1] | — | 0.164 | 0.0297 | 10 | ¹H NMR (400 MHz, DMSO) δ 8.64-8.44 (m, 1H), 7.86-7.68 (m, 4H), 7.60 (dd, J = 8.6, 6.1 Hz, 2H), 6.85- 6.62 (m, 1H), 6.22- 5.99 (m, 1H), 5.75- 5.58 (m, 1H), 3.85- 3.62 (m, 3H), 3.59- 3.37 (m, 5H), 1.90- 1.74 (m, 2H). |
| 28 | 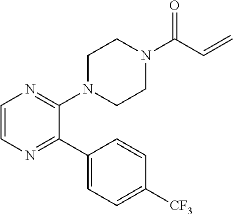 [SMILES: C=CC(=O)N1CCN(c2nccnc2-c2ccc(C(F)(F)F)cc2)CC1] | — | 0.0904 | 0.0114 | >10 | ¹H NMR (400 MHz, DMSO) δ 8.31-8.21 (m, 2H), 8.14 (d, J = 8.1 Hz, 2H), 7.85 (d, J = 8.2 Hz, 2H), 6.77 (dd, J = 16.6, 10.4 Hz, 1H), 6.10 (dd, J = 16.7, 2.4 Hz, 1H), 5.66 (dd, J = 10.4, 2.4 Hz, 1H), 3.58 (s, 4H), 3.12 (d, J = 15.6 Hz, 4H). |
| 29 | 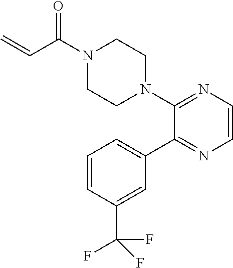 [SMILES: C=CC(=O)N1CCN(c2nccnc2-c2cccc(C(F)(F)F)c2)CC1] | — | >20 | >20 | >20 | ¹H NMR (400 MHz, DMSO) δ 8.29 (d, J = 2.5 Hz, 1H), 8.27- 8.23 (m, 3H), 7.87- 7.70 (m, 2H), 6.77 (dd, J = 16.6, 10.4 Hz, 1H), 6.10 (dd, J = 16.7, 2.4 Hz, 1H), 5.67 (dd, J = 10.4, 2.4 Hz, 1H), 3.56 (d, J = 12.8 Hz, 4H), 3.10 (d, J = 6.5 Hz, 4H). |
| 30 | 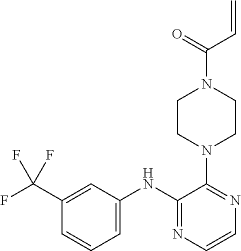 [SMILES: C=CC(=O)N1CCN(c2nccnc2Nc2cccc(C(F)(F)F)c2)CC1] | — | >1 | 0.0199 | >10 | ¹H NMR (400 MHz, DMSO) δ 8.54 (s, 1H), 8.11 (t, J = 2.0 Hz, 1H), 8.02 (d, J = 8.2 Hz, 1H), 7.90-7.75 (m, 2H), 7.52 (t, J = 8.0 Hz, 1H), 7.33-7.25 (m, 1H), 6.85 (dd, J = 16.7, 10.5 Hz, 1H), 6.14 (dd, J = 16.7, 2.4 Hz, 1H), 5.71 (dd, J = 10.4, 2.4 Hz, 1H), 3.78 (d, J = 13.6 Hz, 4H), 3.13 (s, 4H). |
| 31 | 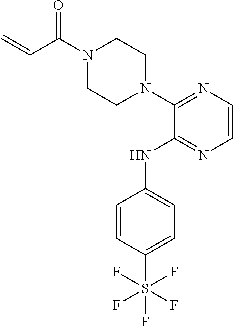 [SMILES: C=CC(=O)N1CCN(c2nccnc2Nc2ccc(S(F)(F)(F)(F)F)cc2)CC1] | 0.018 | 0.01552 | 0.002062 | >5 | ¹H NMR (400 MHz, DMSO) δ 8.72 (s, 1H), 7.92-7.83 (m, 4H), 7.83-7.74 (m, 2H), 6.84 (dd, J = 16.7, 10.5 Hz, 1H), 6.14 (dd, J = 16.7, 2.4 Hz, 1H), 5.71 (dd, J = 10.4, 2.4 Hz, 1H), 3.77 (d, J = 12.5 Hz, 4H), 3.15 (d, J = 6.1 Hz, 4H). |
| 32 | 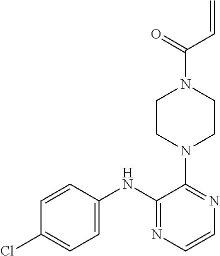 [SMILES: C=CC(=O)N1CCN(c2nccnc2Nc2ccc(Cl)cc2)CC1] | — | 0.059 | 0.004669 | 20 | ¹H NMR (400 MHz, DMSO) δ 8.31 (s, 1H), 7.81 (d, J = 2.8 Hz, 1H), 7.78-7.66 (m, 3H), 7.38-7.23 (m, 2H), 6.84 (dd, J = 16.7, 10.5 Hz, 1H), 6.14 (dd, J = 16.7, 2.4 Hz, 1H), 5.71 (dd, J = 10.4, 2.4 Hz, 1H), 3.77 (d, J = 9.7 Hz, 4H), 3.10 (t, J = 4.6 Hz, 4H). |
| 33 | 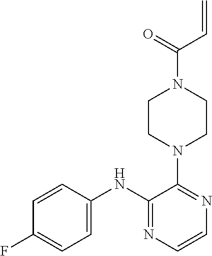 [SMILES: C=CC(=O)N1CCN(c2nccnc2Nc2ccc(F)cc2)CC1] | — | 0.101 | 0.01019 | >20 | ¹H NMR (400 MHz, DMSO) δ 8.20 (s, 1H), 7.77 (d, J = 2.8 Hz, 1H), 7.74-7.63 (m, 3H), 7.14 (t, J = 8.9 Hz, 2H), 6.85 (dd, J = 16.7, 10.4 Hz, 1H), 6.14 (dd, J = 16.7. 2.4 Hz, 1H), 5.71 (dd, J = 10.4, 2.4 Hz, 1H), 3.78 (d, J = 11.7 Hz, 4H), 3.17- 2.95 (m, 4H). |
| 34 | 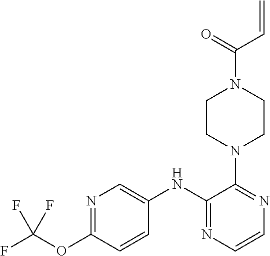 [SMILES: C=CC(=O)N1CCN(c2nccnc2Nc2ccc(OC(F)(F)F)nc2)CC1] | 0.006 | 0.007 | 0.002 | >10 | ¹H NMR (400 MHz, DMSO) δ 8.39 (s, 1H), 7.87-7.69 (m, 4H), 7.35-7.26 (m, 2H), 6.86 (dd, J = 16.7, 10.4 Hz, 1H), 6.16 (dd, J = 16.7, 2.4 Hz, 1H), 5.72 (dd, J = 10.4, 2.4 Hz, 1H), 3.80 (s, 4H), 3.13 (s, 4H). |
| 35 | 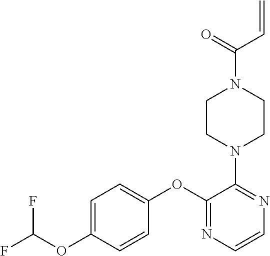 [SMILES: C=CC(=O)N1CCN(c2nccnc2Oc2ccc(OC(F)F)cc2)CC1] | 0.028 | 0.084 | 0.0008 | — | ¹H NMR (400 MHz, DMSO) δ 8.26 (s, 1H), 7.79 (d, J = 2.8 Hz, 1H), 7.72 (dd, J = 9.4, 2.5 Hz, 3H), 7.36- 6.94 (m, 3H), 6.89- 6.76 (m, 1H), 6.16 (dd, J = 16.7, 2.4 Hz, 1H), 5.72 (dd, J = 10.5, 2.4 Hz, 1H), 3.87-3.70 (m, 4H), 3.12 (p, J = 4.0 Hz, 4H). |
| 36 | 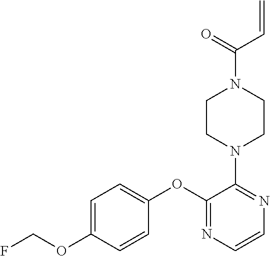 [SMILES: C=CC(=O)N1CCN(c2nccnc2Oc2ccc(OCF)cc2)CC1] | — | 5.04 | 0.005 | — | ¹H NMR (400 MHz, DMSO) δ 8.15 (s, 1H), 7.77 (d, J = 2.8 Hz, 1H), 7.70 (d, J = 2.9 Hz, 1H), 7.68-7.58 (m, 2H), 7.14-7.02 (m, 2H), 6.86 (dd, J = 16.7, 10.4 Hz, 1H), 6.16 (dd, J = 16.7, 2.4 Hz, 1H), 5.89 (s, 1H), 5.83-5.65 (m, 2H), 3.79 (d, J = 12.2 Hz, 4H), 3.11 (d, J = 6.5 Hz, 4H). |
| 37 | 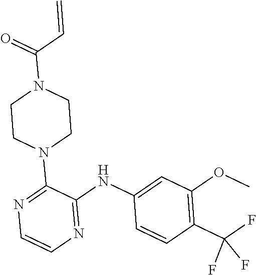 [SMILES: C=CC(=O)N1CCN(c2nccnc2Nc2ccc(C(F)(F)F)c(OC)c2)CC1] | 0.078 | — | — | — | ¹H NMR (400 MHz, DMSO) δ 8.58 (s, 1H), 7.94-7.82 (m, 2H), 7.65 (s, 1H), 7.55- 7.46 (m, 2H), 6.86 (dd, J = 16.7, 10.4 Hz, 1H), 6.16 (dd, J = 16.6, 2.4 Hz, 1H), 5.73 (dd, J = 10.3, 2.4 Hz, 1H), 3.97- 3.67 (m, 7H), 3.16 (d, J = 5.9 Hz, 4H). |
| 38 | 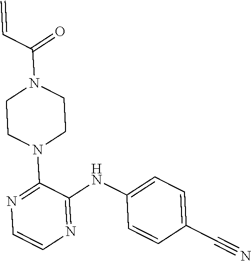 [SMILES: C=CC(=O)N1CCN(c2nccnc2Nc2ccc(C#N)cc2)CC1] | 0.42 | — | — | — | ¹H NMR (400 MHz, DMSO) δ 8.78 (s, 1H), 8.02-7.85 (m, 4H), 7.82-7.66 (m, 2H), 6.92-6.76 (m, 1H), 6.16 (dt, J = 16.7, 1.9 Hz, 1H), 5.72 (dt, J = 10.6, 1.8 Hz, 1H), 3.78 (d, J = 12.8 Hz, 4H), 3.16 (s, 4H). |
| 39 | 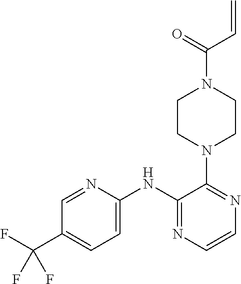 [SMILES: C=CC(=O)N1CCN(c2nccnc2Nc2ccc(C(F)(F)F)cn2)CC1] | 0.015 | 0.016 | 0.005 | >20 | ¹H NMR (400 MHz, DMSO) δ 9.01 (s, 1H), 8.63 (dt, J = 2.1, 1.0 Hz, 1H), 8.15-8.04 (m, 2H), 7.99 (s, 2H), 6.85 (dd, J = 16.7, 10.4 Hz, 1H), 6.15 (dd, J = 16.7, 2.4 Hz, 1H), 5.72 (dd, .J = 10.4, 2.4 Hz, 1H), 3.74 (d, J = 14.9 Hz, 4H), 3.16 (s, 4H). |
| 40 | 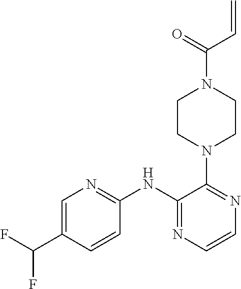 [SMILES: C=CC(=O)N1CCN(c2nccnc2Nc2ccc(C(F)F)cn2)CC1] | — | >20 | 0.004 | — | ¹H NMR (400 MHz, DMSO) δ 8.74 (s, 1H), 8.48 (s, 1H), 8.11 (d, J = 8.7 Hz, 1H), 8.06- 7.92 (m, 3H), 7.25- 6.90 (m, 1H), 6.85 (dd, J = 16.7, 10.4 Hz, 1H), 6.15 (dd, J = 16.7, 2.4 Hz, 1H), 5.72 (dd, J = 10.4, 2.4 Hz, 1H), 3.75 (d, J = 15.6 Hz, 4H), 3.14 (s, 4H). |
| 41 | 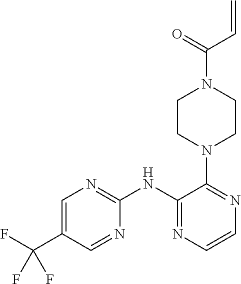 [SMILES: C=CC(=O)N1CCN(c2nccnc2Nc2ncc(C(F)(F)F)cn2)CC1] | — | 0.198 | 0.023 | >20 | ¹H NMR (400 MHz, DMSO) δ 10.36 (s, 1H), 8.89-8.74 (m, 2H), 8.15 (d, J = 2.6 Hz, 1H), 7.99 (d, J = 2.6 Hz, 1H), 6.79 (dd, J = 16.7, 10.5 Hz, 1H), 6.11 (dd, J = 16.6, 2.4 Hz, 1H), 5.69 (dd, J = 10.4, 2.4 Hz, 1H), 3.50 (s, 4H), 3.32-3.28 (m, 4H). |
| 42 | 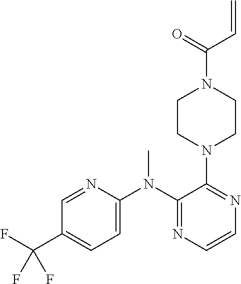 [SMILES: C=CC(=O)N1CCN(c2nccnc2N(C)c2ccc(C(F)(F)F)cn2)CC1] | — | 0.166 | 0.017 | — | ¹H NMR (400 MHz, DMSO) δ 8.61-8.52 (m, 1H), 8.22 (d, J = 2.5 Hz, 1H), 8.05 (d, J = 2.5 Hz, 1H), 7.87 (dd, J = 9.0, 2.6 Hz, 1H), 6.74 (dd, J = 9.7, 4.2 Hz, 2H), 6.17-6.02 (m, 1H), 5.67 (dd, J = 10.4, 2.3 Hz, IH), 3.47 (s, 3H), 3.44-3.39 (m, 4H), 3.22 (dd, J = 6.3, 3.6 Hz, 4H). |
| 43 | 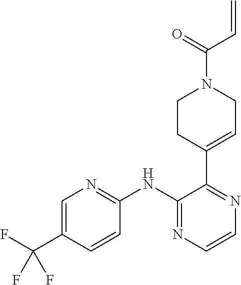 [SMILES: C=CC(=O)N1CC=C(c2nccnc2Nc2ccc(C(F)(F)F)cn2)CC1] | 0.011 | 0.006 | 0.001 | — | ¹H NMR (400 MHz, DMSO) δ 9.56 (d, J = 12.2 Hz, 1H), 8.53 (s, 1H), 8.28 (dd, J = 11.1, 2.7 Hz, 2H), 8.03 (dd, J = 9.1, 2.6 Hz, 1H), 7.78(dd, J = 15.9, 8.8 Hz, 1H), 6.98-6.61 (m, 1H), 6.29-6.08 (m, 2H), 5.71 (ddd, J = 10.3, 4.8, 2.4 Hz, 1H), 4.22-4.03 (m, 2H), 3.74 (dt, J = 16.6, 5.6 Hz, 2H), 2.66-2.53 (m, 2H). |
| 44 | 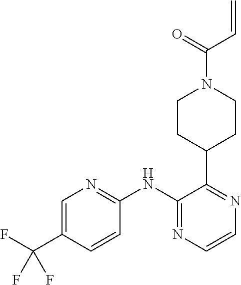 [SMILES: C=CC(=O)N1CCC(c2nccnc2Nc2ccc(C(F)(F)F)cn2)CC1] | 0.020 | 0.012 | 0.0014 | — | ¹H NMR (400 MHz, DMSO) δ 9.80 (s, 1H), 8.60 (dt, J = 2.0, 1.0 Hz, 1H), 8.33-8.15 (m, 2H), 8.04 (dd, J = 9.0, 2.6 Hz, 1H), 7.84 (d, J = 8.9 Hz, 1H), 6.85 (dd, J = 16.7, 10.5 Hz, 1H), 6.12 (dd, J = 16.7, 2.5 Hz, 1H), 5.68 (dd, J = 10.4, 2.5 Hz, 1H), 4.56 (d, J = 12.9 Hz, 1H), 4.18 (d, J = 13.7 Hz, 1H), 3.63- 3.47 (m, 1H), 3.25- 3.11 (m, 1H), 2.77 (t, J = 12.5 Hz, 1H), 1.86 (d, J = 12.8 Hz, 2H), |
| | | | | | | 1.75-1.48 (m, 2H). |
| 45 | 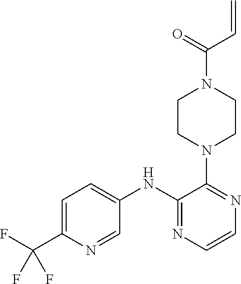 [SMILES: C=CC(=O)N1CCN(c2nccnc2Nc2ccc(C(F)(F)F)nc2)CC1] | 0.050 | 0.040 | 0.003294 | >20 | ¹H NMR (400 MHz, DMSO) δ 9.03 (d, J = 2.5 Hz, 1H), 8.88 (s, 1H), 8.39 (dd, J = 8.7, 2.5 Hz, 1H), 7.99-7.86 (m, 2H), 7.83 (d, J = 8.7 Hz, 1H), 6.86 (dd, J = 16.7, 10.5 Hz, 1H), 6.16 (dd, J = 16.7, 2.4 Hz, 1H), 5.73 (dd, J = 10.4, 2.4 Hz, 1H), 3.92- 3.69 (m, 4H), 3.19 (d, J = 6.1 Hz, 4H). |
| 46 | 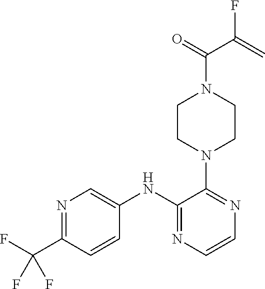 [SMILES: C=C(F)C(=O)N1CCN(c2nccnc2Nc2ccc(C(F)(F)F)nc2)CC1] | 0.452 | 1.63 | 0.0006 | — | ¹H NMR (400 MHz, DMSO) δ 9.03 (d, J = 2.5 Hz, 1H), 8.88 (s, 1H), 8.39 (dd, J = 8.7, 2.5 Hz, 1H), 7.95- 7.87 (m, 2H), 7.84 (d, J = 8.7 Hz, 1H), 5.41- 5.13 (m, 2H), 3.78 (t, J = 4.8 Hz, 4H), 3.23 (t, J =5 .1 Hz, 4H). |
| 47 | 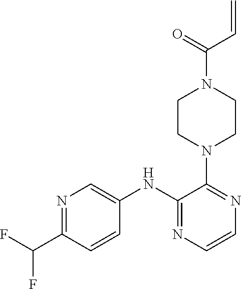 [SMILES: C=CC(=O)N1CCN(c2nccnc2Nc2ccc(C(F)F)nc2)CC1] | — | 0.043 | 0.004 | — | ¹H NMR (400 MHz, DMSO) δ 8.96 (d, J = 2.5 Hz, 1H), 8.70 (s, 1H), 8.32 (dd, J = 8.5, 2.6 Hz, 1H), 7.97- 7.82 (m, 2H), 7.65 (d, J = 8.6 Hz, 1H), 7.05- 6.72 (m, 2H), 6.16 (dd, J = 16.7, 2.4 Hz, 1H), 5.73 (dd, J = 10.4, 2.4 Hz, 1H), 3.80 (d, J = 13.1 Hz, 4H), 3.17 (s, 4H). |
| 48 | 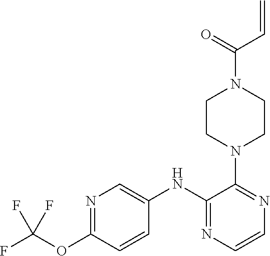 [SMILES: C=CC(=O)N1CCN(c2nccnc2Nc2ccc(OC(F)(F)F)nc2)CC1] | 0.032 | 0.023 | 0.0007 | — | ¹H NMR (400 MHz, DMSO) δ 8.65 (d, J = 2.8 Hz, 1H), 8.58 (s, 1H), 8.30 (dd, J = 8.8, 2.8 Hz, 1H), 7.82 (q, J = 2.8 Hz, 2H), 7.30 (d, J = 8.8 Hz, 1H), 6.87 (dd, J = 16.7, 10.4 Hz, 1H), 6.16 (dd, J = 16.7, 2.4 Hz, 1H), 5.73 (dd, J = 10.4, 2.4 Hz, 1H), 3.80 (s, 4H), 3.16 (s, 4H). |
| 49 | 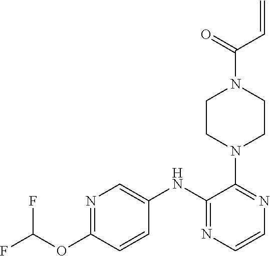 [SMILES: C=CC(=O)N1CCN(c2nccnc2Nc2ccc(OC(F)F)nc2)CC1] | 0.031 | 0.055 | 0.002 | — | ¹H NMR (400 MHz, DMSO) δ 8.53 (d, J = 2.7 Hz, 1H), 8.41 (s, 1H), 8.18 (dd, J = 8.8, 2.8 Hz, 1H), 7.87-7.43 (m, 3H), 7.09 (d, J = 8.8 Hz, 1H), 6.87 (dd, J = 16.7, 10.4 Hz, 1H), 6.16 (dd, J = 16.7, 2.4 Hz, 1H), 5.73 (dd, J = 10.4, 2.4 Hz, 1H), 3.80 (s, 4H), 3.15 (d, J = 6.1 Hz, 4H). |
| 50 | 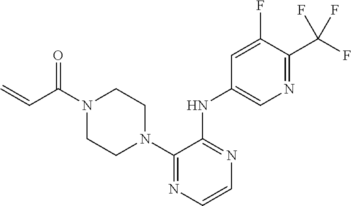 [SMILES: C=CC(=O)N1CCN(c2nccnc2Nc2cnc(C(F)(F)F)c(F)c2)CC1] | 0.026 | 0.034 | 0.0031 | — | ¹H NMR (400 MHz, DMSO) δ 9.20 (d, J = 7.4 Hz, 1H), 8.84 (d, J = 9.0 Hz, 1H), 8.42 (d, J = 13.3 Hz, 1H), 7.96 (d, J = 7.8 Hz, 2H), 6.85 (d, J = 14.0 Hz, 1H), 6.16 (d, J = 10.8 Hz, 1H), 5.73 (d, J = 11.4 Hz, 1H), 3.78 (s, 4H), 3.19 (s, 4H). |
| 51 | 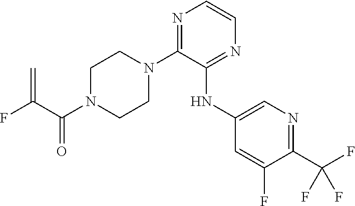 [SMILES: C=C(F)C(=O)N1CCN(c2nccnc2Nc2cnc(C(F)(F)F)c(F)c2)CC1] | — | 0.331 | 0.0067 | — | ¹H NMR (400 MHz, DMSO) δ 9.20 (s, 1H), 8.85 (s, 1H), 8.42 (d, J = 13.6 Hz, 1H), 7.97 (d, J = 8.4 Hz, 2H), 5.39- 5.14 (m, 2H), 3.76 (s, 4H), 3.23 (s, 4H). |
| 52 | 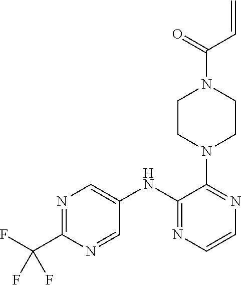 [SMILES: C=CC(=O)N1CCN(c2nccnc2Nc2cnc(C(F)(F)F)nc2)CC1] | — | 0.128 | 0.01053 | >20 | ¹H NMR (400 MHz, DMSO) δ 9.34 (s, 2H), 9.10 (s, 1H), 7.99- 7.87 (m, 2H), 6.87 (dd, J = 16.7, 10.4 Hz, 1H), 6.16 (dd, J = 16.7, 2.4 Hz, 1H), 5.80-5.65 (m, 1H), 3.79 (d, J = 11.8 Hz, 4H), 3.21 (d, J = 6.4 Hz, 4H). |
| 53 | 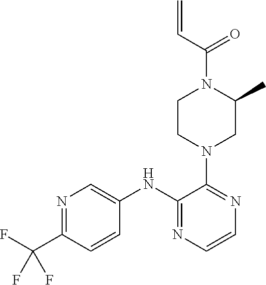 [SMILES: C=CC(=O)N1CCN(c2nccnc2Nc2ccc(C(F)(F)F)nc2)C[C@@H]1C] | — | 0.076 | 0.0036 | — | ¹H NMR (400 MHz, DMSO) δ 8.96 (d, J = 2.5 Hz, 1H), 8.60 (s, 1H), 8.32 (dd, J = 8.7, 2.5 Hz, 1H), 7.91 (s, 2H), 7.85 (d, J = 8.6 Hz, 1H), 6.83 (dd, J = 16.7, 10.4 Hz, 1H), 6.15 (dd, J = 16.6, 2.4 Hz, 1H), 5.72 (dd, J = 10.5, 2.4 Hz, 1H), 4.88- 3.97 (m, 2H), 3.76- 3.60 (m, 1H), 3.62- 3.35 (m, 2H), 3.01 (d, J = 12.8 Hz, 1H), 2.70 (s, 1H), 1.33 (d, J = 6.7 Hz, 3H). |
| 54 | 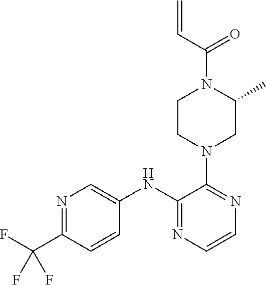 [SMILES: C=CC(=O)N1CCN(c2nccnc2Nc2ccc(C(F)(F)F)nc2)C[C@H]1C] | — | 0.192 | 0.009 | — | ¹H NMR (400 MHz, DMSO) δ 8.96 (d, J = 2.5 Hz, 1H), 8.60 (s, 1H), 8.32 (dd, J = 8.7, 2.6 Hz, 1H), 7.91 (s, 2H), 7.85 (d, J = 8.7 Hz, 1H), 6.83 (dd, J = 16.7, 10.5 Hz, 1H), 6.15 (dd, J = 16.7, 2.4 Hz, 1H), 5.72 (dd, J = 10.5, 2.4 Hz, 1H), 3.66 (d, J = 12.1 Hz, 2H), 3.56-3.36 (m, 2H), 3.02 (s, 1H), 2.69 (s, 1H), 1.46-1.22 (m, 3H). |
| 55 | 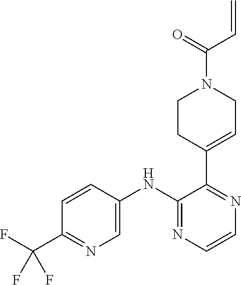 [SMILES: C=CC(=O)N1CC=C(c2nccnc2Nc2ccc(C(F)(F)F)nc2)CC1] | 0.058 | 0.030 | 0.004 | — | ¹H NMR (400 MHz, DMSO) δ 9.08-8.89 (m, 2H), 8.30 (d, J = 8.7 Hz, 1H), 8.16 (dt, J = 14.0, 2.4 Hz, 2H), 7.82 (d, J = 8.6 Hz, 1H), 7.00-6.72 (m, 1H), 6.37 (d, J = 14.9 Hz, 1H), 6.19 (ddd, J = 16.7, 9.6, 2.4 Hz, 1H), 5.75 (td, J = 9.6, 2.4 Hz, 1H), 4.39-4.17 (m, 2H), 3.87-3.74 (m, 2H), 2.70-2.55 (m, 2H). |
| 56 | 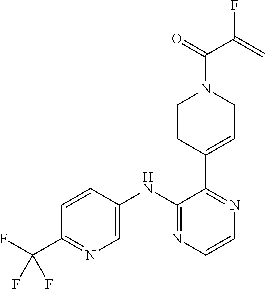 [SMILES: C=C(F)C(=O)N1CC=C(c2nccnc2Nc2ccc(C(F)(F)F)nc2)CC1] | — | 0.145 | 0.0165 | — | ¹H NMR (400 MHz, DMSO) δ 9.04-8.88 (m, 2H), 8.30 (d, J = 8.3 Hz, 1H), 8.22- 7.99 (m, 2H), 7.82 (d, J = 8.7 Hz, 1H), 6.37 (s, 1H), 5.44-5.17 (m, 2H), 4.38-4.15 (m, 2H), 3.79 (t, J = 5.8 Hz, 2H), 2.67 (s, 2H). |
| 57 | 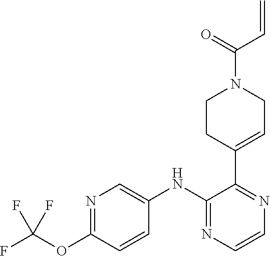 [SMILES: C=CC(=O)N1CC=C(c2nccnc2Nc2ccc(OC(F)(F)F)nc2)CC1] | 0.036 | — | — | — | ¹H NMR (400 MHz, DMSO) δ 8.70 (d, J = 4.0 Hz, 1H), 8.55 (s, 1H), 8.21 (d, J = 8.5 Hz, 1H), 8.06 (d, J = 12.2 Hz, 2H), 7.28 (d, J = 8.8 Hz, IH), 6.99- 6.75 (m, 1H), 6.35 (d, J = 14.0 Hz, 1H), 6.18 (dd, J = 17.1, 9.1 Hz, 1H), 5.75 (t, J = 9.4 Hz, 1H), 4.39-4.18 (m, 2H), 3.81 (d, J = 8.1 Hz, 2H), 2.62 (s, 2H). |
| 58 | 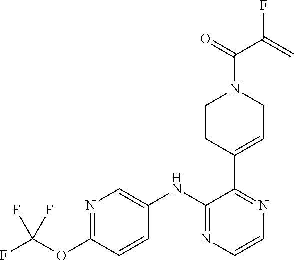 [SMILES: C=C(F)C(=O)N1CC=C(c2nccnc2Nc2ccc(OC(F)(F)F)nc2)CC1] | 0.283 | — | — | — | ¹H NMR (400 MHz, DMSO) δ 8.71 (s, 1H), 8.56 (dd, J = 2.7, 1.3 Hz, 1H), 8.20 (dd, J = 8.8, 2.6 Hz, 1H), 8.11- 7.98 (m, 2H), 7.28 (d, J = 8.8 Hz, 1H), 6.36 (s, 1H), 5.45-5.13 (m, 2H), 4.36-4.15 (m, 2H), 3.78 (t, J = 5.7 Hz, 2H), 2.65 (s, 2H). |
| 59 | 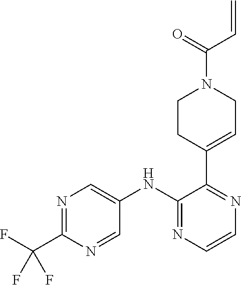 [SMILES: C=CC(=O)N1CC=C(c2nccnc2Nc2cnc(C(F)(F)F)nc2)CC1] | 0.1 | — | — | — | ¹H NMR (400 MHz, DMSO) δ 9.41-9.10 (m, 3H), 8.29-8.01 (m, 2H), 7.01-6.78 (m, 1H), 6.41 (d, J = 12.7 Hz, 1H), 6.19 (dd, J = 16.5, 10.4 Hz, 1H), 5.75 (dd, J = 11.2, 8.8 Hz, 1H), 4.38-4.19 (m, 2H), 3.91-3.74 (m, 2H), 3.32 (s, 1H), 2.68-2.57 (m, 2H). |
| 60 | 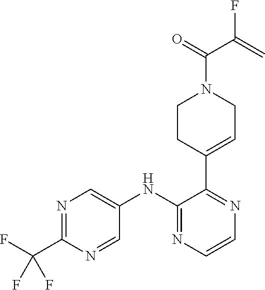 [SMILES: C=C(F)C(=O)N1CC=C(c2nccnc2Nc2cnc(C(F)(F)F)nc2)CC1] | 1.31 | — | — | — | ¹H NMR (400 MHz, DMSO) δ 9.40-9.10 (m, 3H), 8.33-8.09 (m, 2H), 6.42 (s, 1H), 5.44-5.13 (m, 2H), 4.40-4.17 (m, 2H), 3.80 (t, J = 5.6 Hz, 2H), 3.32 (s, 1H), 2.68 (s, 2H). |
| 61 | 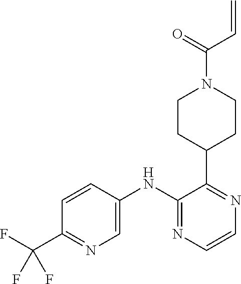 [SMILES: C=CC(=O)N1CCC(c2nccnc2Nc2ccc(C(F)(F)F)nc2)CC1] | — | 0.257 | 0.005 | — | ¹H NMR (400 MHz, DMSO) δ 9.05 (s, 1H), 8.98 (d, J = 2.6 Hz, 1H), 8.33 (dd, J = 8.7, 2.5 Hz, 1H), 8.21-8.02 (m, 2H), 7.84 (d, J = 8.6 Hz, 1H), 6.87 (dd, J = 16.7, 10.5 Hz, 1H), 6.13 (dd, J = 16.7, 2.5 Hz, 1H), 5.69 (dd, J = 10.5, 2.5 Hz, 1H), 4.60 (d, J = 12.9 Hz, 1H), 4.22 (d, J = 13.5 Hz, 1H), 3.51 (dtd, J = 11.6, 7.7, 4.0 Hz, 1H), 3.26 (t, J = 13.2 Hz, 1H), 2.94-2.74 (m, 1H), 1.90 (d, J = 13.0 Hz, |
| | | | | | | 2H), 1.76-1.50 (m, |
| | | | | | | 2H). |
| 62 | 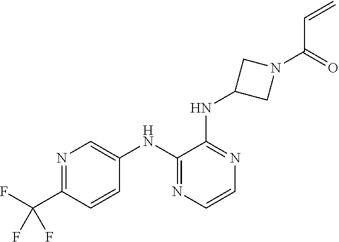 [SMILES: C=CC(=O)N1CC(Nc2nccnc2Nc2ccc(C(F)(F)F)nc2)C1] | — | 8.67 | 0.0107 | — | ¹H NMR (400 MHz, DMSO) δ 8.87 (d, J = 8.4 Hz, 2H), 8.41 (d, J = 11.1 Hz, 1H), 7.82 (q, J = 6.3 Hz, 1H), 7.71- 7.47 (m, 2H), 7.28 (s, 1H), 6.36 (d, J = 14.6 Hz, 1H), 6.14 (d, J = 14.0 Hz, 1H), 5.70 (d, J = 10.8 Hz, 1H), 4.62 (d, J = 9.9 Hz, 2H), 4.32 (s, 1H), 4.07 (s, 1H), 3.91 (s, 1H). |
| 63 | 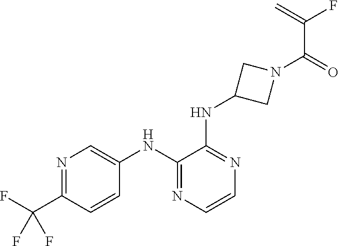 [SMILES: C=C(F)C(=O)N1CC(Nc2nccnc2Nc2ccc(C(F)(F)F)nc2)C1] | 1.7 | 1.5 | 0.0048 | — | ¹H NMR (400 MHz, DMSO) δ 8.97-8.80 (m, 2H), 8.40 (dd, J = 8.7, 2.6 Hz, 1H), 7.83 (d, J = 8.7 Hz, 1H), 7.66 (d, J = 2.9 Hz, 1H), 7.54 (d, J = 3.0 Hz, 1H), 7.29 (d, J = 5.5 Hz, 1H), 5.61- 5.41 (m, IH), 5.33 (dd, J = 16.6, 3.6 Hz., 1H), 4.88-4.61 (m, 2H), 4.46-4.33 (m, 1H), 4.29-4.17 (m, 1H), 3.95 (dd, J = 10.7, 4.8 Hz, 1H). |
| 64 | 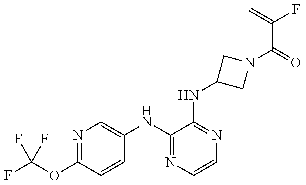 [SMILES: C=C(F)C(=O)N1CC(Nc2nccnc2Nc2ccc(OC(F)(F)F)nc2)C1] | — | 3.12 | 1.68 | — | ¹H NMR (400 MHz, DMSO) δ 8.64-8.51 (m, 2H), 8.29 (dd, J = 8.9, 2.8 Hz, 1H), 7.58 (d, J = 3.0 Hz, 1H), 7.47 (d, J = 3.0 Hz, 1H), 7.29 (d, J = 8.8 Hz, 1H), 7.19 (d, J = 5.5 Hz, 1H), 5.57- 5.41 (m, 1H), 5.32 (dd, J = 16.6, 3.6 Hz, 1H), 4.85-4.62 (m, 2H), |
| | | | | | | 4.44-4.33 (m, 1H), |
| | | | | | | 4.23 (s, 1H), 4.01- |
| | | | | | | 3.88 (m, 1H). |
| 65 | 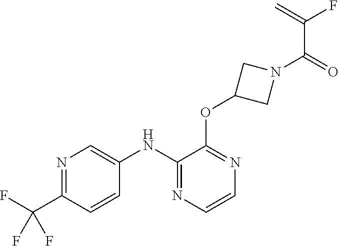 [SMILES: C=C(F)C(=O)N1CC(Oc2nccnc2Nc2ccc(C(F)(F)F)nc2)C1] | — | 1.85 | 0.0186 | — | ¹H NMR (400 MHz, DMSO) δ 9.33 (s, 1H), 9.17 (dd, J = 5.6, 2.5 Hz, 1H), 8.81-8.54 (m, 1H), 7.93-7.62 (m, 2H), 6.74-6.53 (m, 1H), 5.77-5.28 (m, 3H), 4.85 (s, 1H), 4.65-4.41 (m, 1H), 4.29-4.11 (m, 1H), 3.98 (d, J = 14.4 Hz, 1H), 3.77 (q, J = 17.3 Hz, 1H). |
| 66 | 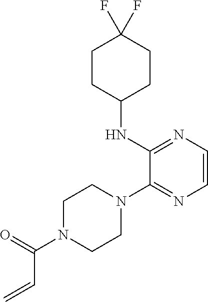 [SMILES: C=CC(=O)N1CCN(c2nccnc2NC2CCC(F)(F)CC2)CC1] | — | 0.347 | 0.0192 | >20 | ¹H NMR (400 MHz, DMSO) δ 7.70 (d, J = 2.9 Hz, 1H), 7.47 (d, J = 2.9 Hz, 1H), 6.86 (dd, J = 16.7, 10.5 Hz, 1H), 6.15 (dd, J = 16.7, 2.4 Hz, 1H), 5.92 (d, J = 7.8 Hz, 1H), 5.71 (dd, J = 10.4, 2.4 Hz, 1H), 4.03 (s, 1H), 3.76 (d, J = 14.1 Hz, 4H), 3.02 (s, 4H), 2.16-1.84 (m, 6H), 1.74 (t, J = 11.3 Hz, 2H). |
| 67 | 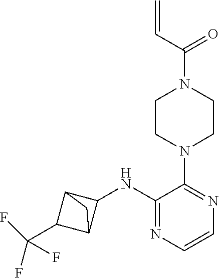 [SMILES: C=CC(=O)N1CCN(c2nccnc2NC2C3CC2C3C(F)(F)F)CC1] | 1.45 | 0.079 | 0.0029 | — | ¹H NMR (400 MHz, DMSO) δ 7.76 (d, J = 2.9 Hz, 1H), 7.57 (d, J = 2.8 Hz, 1H), 7.05 (s, 1H), 6.85 (dd, J = 16.7, 10.4 Hz, 1H), 6.15 (dd, J = 16.7, 2.4 Hz, 1H), 5.72 (dd, J = 10.4, 2.4 Hz, 1H), 3.75 (d, J = 12.1 Hz, 4H), 3.00 (s, 4H), 2.37 (s, 6H). |
| 68 | 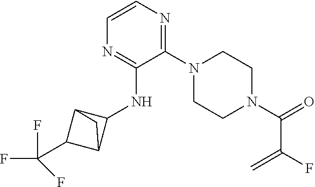 [SMILES: C=C(F)C(=O)N1CCN(c2nccnc2NC2C3CC2C3C(F)(F)F)CC1] | — | 0.708 | 0.011 | — | ¹H NMR (400 MHz, DMSO) δ 7.77 (d, J = 2.8 Hz, 1H), 7.57 (d, J = 2.9 Hz, 1H), 7.07 (s, 1H), 5.36-5.13 (m, 2H), 3.74 (t, J = 5.0 Hz, 4H), 3.04 (t, J = 5.1 Hz, 4H), 2.37 (s, 6H). |
| 69 | 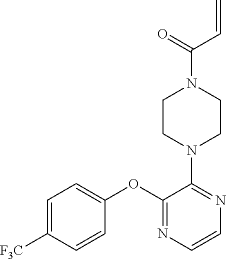 [SMILES: C=CC(=O)N1CCN(c2nccnc2Oc2ccc(C(F)(F)F)cc2)CC1] | — | 0.0115 | 0.0009 | >20 | ¹H NMR (400 MHz, DMSO) δ 8.01 (d, J = 2.8 Hz, 1H), 7.82 (d, J = 8.5 Hz, 2H), 7.61 (d, J = 2.8 Hz, 1H), 7.45 (d, J = 8.4 Hz, 2H), 6.85 (dd, J = 16.7, 10.4 Hz, 1H), 6.16 (dd, J = 16.7, 2.4 Hz, 1H), 5.72 (dd, J = 10.4, 2.4 Hz, 1H), 3.74 (s, 4H), 3.58 (s, 4H). |
| 70 | 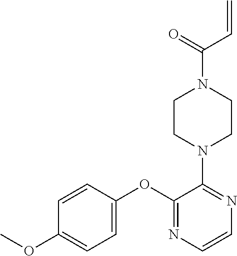 [SMILES: C=CC(=O)N1CCN(c2nccnc2Oc2ccc(OC)cc2)CC1] | — | 0.028 | 0.003 | — | ¹H NMR (400 MHz, DMSO) δ 7.90 (d, J = 2.8 Hz, 1H), 7.55 (d, J = 2.7 Hz, 1H), 7.18- 7.05 (m, 2H), 7.04- 6.92 (m, 2H), 6.86 (dd, J = 16.7, 10.4 Hz, 1H), 6.16 (dd, J = 16.7, 2.4 Hz, 1H), 5.73 (dd, J = 10.4, 2.3 Hz, 1H), 3.78 (s, 3H), 3.77-3.68 (m, 4H), 3.63-3.48 (m, 4H). |
| 71 | 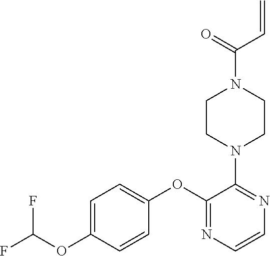 [SMILES: C=CC(=O)N1CCN(c2nccnc2Oc2ccc(OC(F)F)cc2)CC1] | 0.007 | 0.008 | 0.0013 | — | ¹H NMR (400 MHz, DMSO) δ 7.95 (d, J = 2.8 Hz, 1H), 7.58 (d, J = 2.8 Hz, 1H), 7.48- 7.03 (m, 5H), 6.86 (dd, J = 16.7, 10.4 Hz, 1H), 6.16 (dd, J = 16.7, 2.4 Hz, 1H), 5.73 (dd, J = 10.4, 2.4 Hz, 1H), 3.83- 3.64 (m, 4H), 3.58 (t, J = 5.1 Hz, 4H). |
| 72 | 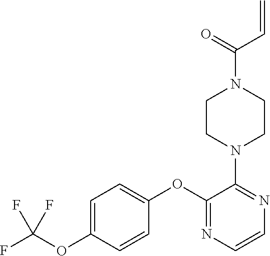 [SMILES: C=CC(=O)N1CCN(c2nccnc2Oc2ccc(OC(F)(F)F)cc2)CC1] | 0.011 | 0.005 | 0.00052 | — | ¹H NMR (400 MHz, DMSO) δ 7.97 (d, J = 2.8 Hz, 1H), 7.59 (d, J = 2.8 Hz, 1H), 7.50- 7.39 (m, 2H), 7.41- 7.29 (m, 2H), 6.85 (dd, J = 16.8, 10.5 Hz, 1H), 6.16 (dd, J = 16.7, 2.4 Hz, 1H), 5.73 (dd, J = 10.4, 2.4 Hz, 1H), 3.72 (d, J = 17.7 Hz, 4H), 3.57 (d, J = 6.8 Hz, 4H). |
| 73 | 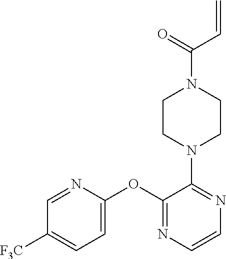 [SMILES: C=CC(=O)N1CCN(c2nccnc2Oc2ccc(C(F)(F)F)cn2)CC1] | 0.032 | 0.025 | 0.003 | >20 | 1H NMR (400 MHz, DMSO) δ 8.69 (d, J = 3.4 Hz, 1H), 8.36 (dd, J = 8.5, 3.1 Hz, 1H), 8.15 (q, J = 2.5 Hz, 1H), 7.77 (q, J = 2.5 Hz, 1H), 7.49 (dd, J = 9.0, 3.3 Hz, 1H), 6.81 (dd, J = 16.7, 10.6 Hz, 1H), 6.13 (dt, J = 16.7, 2.2 Hz, 1H), 5.70 (dt, J = 10.4, 2.3 Hz, 1H), 3.69- 3.45 (m, 8H). |
| 74 | 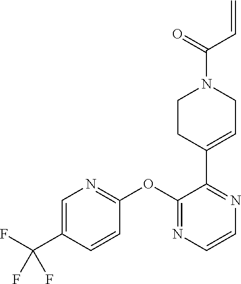 [SMILES: C=CC(=O)N1CC=C(c2nccnc2Oc2ccc(C(F)(F)F)cn2)CC1] | 0.121 | 0.449 | 0.0024 | — | ¹H NMR (400 MHz, DMSO) δ 8.68 (d, J = 2.6 Hz, 1H), 8.65- 8.54 (m, 1H), 8.37 (dd, J = 8.7, 2.6 Hz, 1H), 8.33-8.21 (m, 1H), 7.51 (d, J = 8.6 Hz, 1H), 6.93-6.69 (m, 2H), 6.13 (dd, J = 16.8, 2.4 Hz, 1H), 5.70 (dd, J = 10.4, 2.4 Hz., 1H), 4.38-4.12 (m, 2H), 3.71 (dt, J = 16.3, 5.7 Hz, 2H), 2.77-2.54 (m, 2H). |
| 75 | 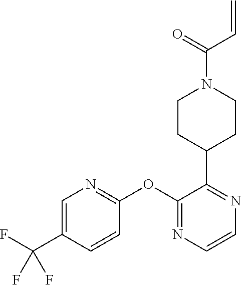 [SMILES: C=CC(=O)N1CCC(c2nccnc2Oc2ccc(C(F)(F)F)cn2)CC1] | — | 0.112 | 0.0027 | — | ¹H NMR (400 MHz, DMSO) δ 8.84 (d, J = 2.4 Hz, 1H), 8.67- 8.55 (m, 2H), 7.92 (dd, J = 9.7, 2.8 Hz, 1H), 6.84 (td, J = 10.7, 5.0 Hz, 1H), 6.75 (d, J = 9.7 Hz, 1H), 6.12 (dd, J = 16.7, 2.5 Hz, 1H), 5.68 (d, J = 10.6 Hz, 1H), 4.60-4.40 (m, 1H), 4.26-4.02 (m, 1H), 3.09 (t, J = 13.0 Hz, 1H), 2.93-2.79 (m, 1H), 2.76-2.61 (m, 1H), 1.89 (s, 1H), 1.74 (t, J = 14.4 Hz, 2H), 1.59 (d, J = 12.9 |
| | | | | | | Hz, 1H). |
| 76 | 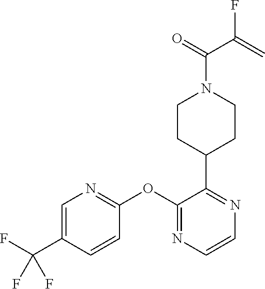 [SMILES: C=C(F)C(=O)N1CCC(c2nccnc2Oc2ccc(C(F)(F)F)cn2)CC1] | 0.104 | 0.424 | — | — | ¹H NMR (400 MHz, DMSO) δ 8.70 (s, 1H), 8.53 (d, J = 2.6 Hz, 1H), 8.38 (d, J = 8.9 Hz, 1H), 8.26 (d, J = 2.6 Hz, 1H), 7.52 (d, J = 8.7 Hz, 1H), 5.33- 5.08 (m, 2H), 3.31 (s, 2H), 1.94 (d, J = 13.3 Hz, 2H), 1.75 (s, 2H). |
| 77 | 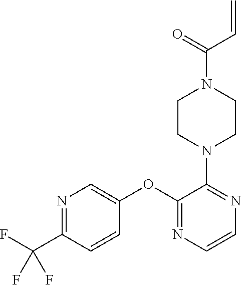 [SMILES: C=CC(=O)N1CCN(c2nccnc2Oc2ccc(C(F)(F)F)nc2)CC1] | — | 0.008 | 0.0018 | — | ¹H NMR (400 MHz, DMSO) δ 8.77 (t, J = 1.7 Hz, 1H), 8.04 (t, J = 2.3 Hz, 3H), 7.61 (d, J = 2.7 Hz, 1H), 6.86 (dd, J = 16.7, 10.4 Hz, 1H), 6.16 (dd, J = 16.7, 2.4 Hz, 1H), 5.73 (dd, J = 10.4, 2.4 Hz, 1H), 3.73 (dd, J = 17.9, 5.7 Hz, 4H), 3.61 (d, J = 6.1 Hz, 4H). |
| 78 | 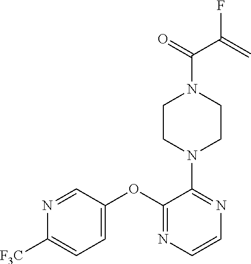 [SMILES: C=C(F)C(=O)N1CCN(c2nccnc2Oc2ccc(C(F)(F)F)nc2)CC1] | 0.009 | 0.032 | 0.0021 | — | ¹H NMR (400 MHz, DMSO) δ 8.77 (s, 1H), 8.04 (d, J = 2.1 Hz, 3H), 7.62 (t, J = 2.3 Hz, 1H), 5.39-5.15 (m, 2H), 3.77-3.63 (m, 8H). |
| 79 | 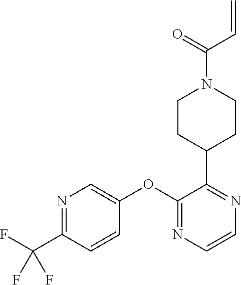 [SMILES: C=CC(=O)N1CCC(c2nccnc2Oc2ccc(C(F)(F)F)nc2)CC1] | 0.028 | 0.017 | 0.002 | — | ¹H NMR (400 MHz, DMSO) δ 8.78 (d, J = 1.7 Hz, 1H), 8.41 (d, J = 2.6 Hz, 1H), 8.07 (dd, J = 9.7, 2.2 Hz, 3H), 6.87 (dd, J = 16.7, 10.4 Hz, 1H), 6.13 (dd, J = 16.7, 2.4 Hz, 1H), 5.70 (dd, J = 10.4, 2.5 Hz, 1H), 4.58 (d, J = 13.0 Hz, 1H), 4.22 (d, J = 13.6 Hz, 1H), 3.51 (tt, J = 11.6, 3.7 Hz, 1H), 3.28 (t, J = 13.0 Hz, 1H), 2.86 (t, J = 12.5 Hz, 1H), 2.11-1.94 (m, 2H), 1.86-1.57 (m, 2H). |
| 80 | 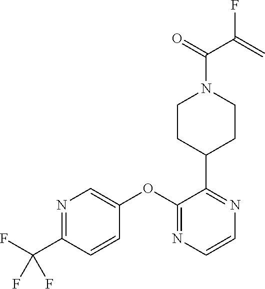 [SMILES: C=C(F)C(=O)N1CCC(c2nccnc2Oc2ccc(C(F)(F)F)nc2)CC1] | — | 0.55 | 0.067 | — | ¹H NMR (400 MHz, DMSO) δ 8.78 (d, J = 1.8 Hz, 1H), 8.42 (d, J = 2.7 Hz, 1H), 8.07 (dd, J = 13.1, 2.2 Hz, 3H), 6.24 (s, 1H), 6.02 (s, 1H), 4.53 (d, J = 13.0 Hz, 1H), 3.96 (d, J = 13.5 Hz, 1H), 3.54 (t, J = 11.6 Hz, 1H), 3.00 (d, J = 12.9 Hz, 1H), 2.17-1.97 (m, 2H), 1.90-1.53 (m, 2H). |
| 81 | 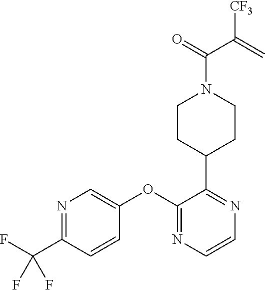 [SMILES: C=C(C(=O)N1CCC(c2nccnc2Oc2ccc(C(F)(F)F)nc2)CC1)C(F)(F)F] | — | 2.97 | 0.0095 | — | ¹H NMR (400 MHz, DMSO) δ 8.78 (s, 1H), 8.42 (d, J = 2.6 Hz, 1H), 8.15-7.97 (m, 3H), 6.24 (s, 1H), 6.02 (s, 1H), 4.53 (d, J = 13.0 Hz, 1H), 3.95 (d, J = 13.6 Hz, 1H), 3.54 (t, J = 11.4 Hz, 1H), 3.33 (s, 1H), 2.98 (t, J = 13.1 Hz, 1H), 2.05 (t, J = 17.8 Hz, 2H), 1.88- 1.65 (m, 2H). |
| 82 | 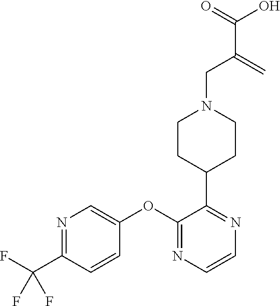 [SMILES: C=C(CN1CCC(c2nccnc2Oc2ccc(C(F)(F)F)nc2)CC1)C(=O)O] | — | >20 | >20 | — | ¹H NMR (400 MHz, DMSO) δ 8.75 (s, 1H), 8.39 (d, J = 9.8 Hz, 1H), 8.04 (d, J = 9.2 Hz, 3H), 5.77 (s, 1H), 5.25 (s, 1H), 3.15 (s, 3H), 3.01 (s, 2H), 2.12 (s, 2H), 1.90 (s, 4H). |
| 83 | 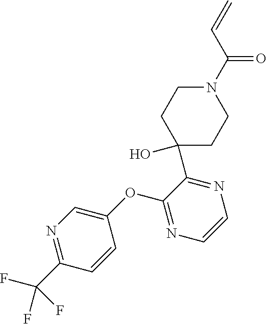 [SMILES: C=CC(=O)N1CCC(O)(c2nccnc2Oc2ccc(C(F)(F)F)nc2)CC1] | 0.263 | — | — | — | ¹H NMR (400 MHz, DMSO-d₆) δ 8.68 (d, J = 2.6 Hz, 1H), 8.43 (d, J = 2.6 Hz, 1H), 8.18 (d, J = 2.6 Hz, 1H), 8.02 (d, J = 8.6 Hz, 1H), 7.93 (dd, J = 8.5, 2.6 Hz, 1H), 6.85 (dd, J = 16.7, 10.5 Hz, 1H), 6.09 (dd, J = 16.7, 2.5 Hz, 1H), 5.66 (dd, J = 10.4, 2.5 Hz, 1H), 5.53 (s, 1H), 4.25 (m, 1H), 3.95 (m, 1H), 3.57 (m, 1H), 3.22 (m, 1H), 2.35-2.05 (m, 4H). |
| 84 | 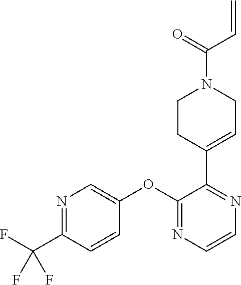 [SMILES: C=CC(=O)N1CC=C(c2nccnc2Oc2ccc(C(F)(F)F)nc2)CC1] | — | 0.0173 | 0.0014 | — | ¹H NMR (400 MHz, DMSO) δ 8.78 (t, J = 1.6 Hz, 1H), 8.47 (t, J = 2.5 Hz, 1H), 8.13- 7.97 (m, 3H), 7.06- 6.76 (m, 2H), 6.17 (dt, J = 16.8, 3.3 Hz, 1H), 5.73 (dd, J = 10.4, 2.4 Hz, 1H), 4.47-4.25 (m, 2H), 3.80 (dt, J = 14.1, 5.6 Hz, 2H), 2.83- 2.63 (m, 2H). |
| 85 | 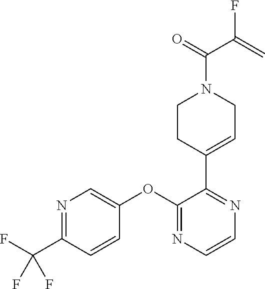 [SMILES: C=C(F)C(=O)N1CC=C(c2nccnc2Oc2ccc(C(F)(F)F)nc2)CC1] | 0.011 | 0.016 | 0.0016 | | ¹H NMR (400 MHz, DMSO) δ 8.78 (s, 1H), 8.47 (d, J = 2.5 Hz, 1H), 8.07 (d, J = 16.8 Hz, 3H), 7.02 (s, 1H), 5.42-5.15 (m, 2H), 4.44-4.17 (m, 2H), 3.78 (t, J = 5.6 Hz, 2H), 2.79 (s, 2H). |
| 86 | 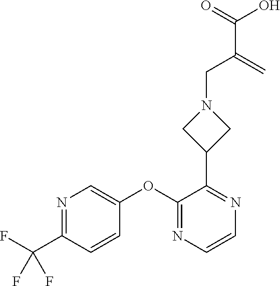 [SMILES: C=C(CN1CC(c2nccnc2Oc2ccc(C(F)(F)F)nc2)C1)C(=O)O] | — | 3.01 | 31.04 | — | ¹H NMR (400 MHz, DMSO) δ 8.74 (1, J = 1.7 Hz, 1H), 8.44 (d, J = 2.7 Hz, 1H), 8.11- 7.95 (m, 3H), 5.70 (d, J = 3.7 Hz, 1H), 5.15 (d, J = 3.6 Hz, 1H), 4.10 (t, J = 7.8 Hz, 1H), 3.70 (t, J = 7.4 Hz, 2H), 3.21 (s, 2H). |
| 87 | 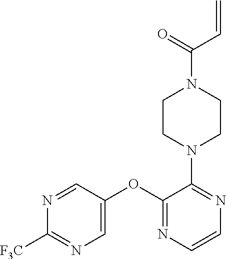 [SMILES: C=CC(=O)N1CCN(c2nccnc2Oc2cnc(C(F)(F)F)nc2)CC1] | 0.047 | 0.025 | 0.0014 | — | ¹H NMR (400 MHz, DMSO) δ 9.18 (s, 2H), 8.08 (d, J = 2.7 Hz, 1H), 7.63 (d, J = 2.7 Hz, 1H), 6.87 (dd, J = 16.7, 10.4 Hz, 1H), 6.17 (dd, J = 16.7, 2.4 Hz, 1H), 5.74 (dd, J = 10.4, 2.4 Hz, 1H), 3.83- 3.67 (m, 4H), 3.63 (dd, J = 6.7, 3.5 Hz, 4H). |
| 88 | 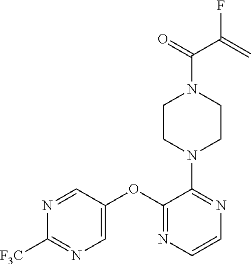 [SMILES: C=C(F)C(=O)N1CCN(c2nccnc2Oc2cnc(C(F)(F)F)nc2)CC1] | 0.027 | 0.047 | 0.0019 | — | ¹H NMR (400 MHz, DMSO) δ 9.17 (s, 2H), 8.08 (d, J = 2.3 Hz, 1H), 7.64 (d, J = 2.4 Hz, 1H), 5.40-5.16 (m, 2H), 3.77-3.65 (m, 8H). |
| 89 | 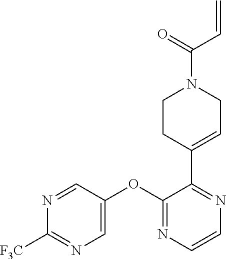 [SMILES: C=CC(=O)N1CC=C(c2nccnc2Oc2cnc(C(F)(F)F)nc2)CC1] | 0.033 | 0.164 | 0.0018 | — | ¹H NMR (400 MHz, DMSO) δ 9.19 (s, 2H), 8.52 (d, J = 2.5 Hz, 1H), 8.11 (s, 1H), 7.09- 6.76 (m, 2H), 6.17 (d, J = 17.0 Hz, 1H), 5.74 (dd, J = 10.5, 2.4 Hz, 1H), 4.48-4.24 (m, 2H), 3.82 (dd, J = 13.7, 7.0 Hz, 2H), 2.79- 2.70 (m, 2H). |
| 90 | 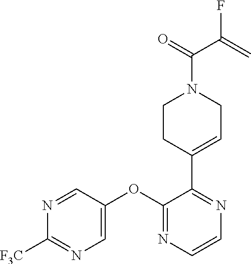 [SMILES: C=C(F)C(=O)N1CC=C(c2nccnc2Oc2cnc(C(F)(F)F)nc2)CC1] | 0.031 | 0.033 | 0.0059 | — | ¹H NMR (400 MHz, DMSO) δ 9.18 (d, J = 8.1 Hz, 2H), 8.52 (d, J = 7.9 Hz, 1H), 8.12 (d, J = 7.4 Hz, 1H), 7.03 (s, 1H), 5.42-5.14 (m, 2H), 4.47-4.18 (m, 2H), 3.78 (d, J = 7.7 Hz, 2H), 2.78 (s, 2H). |
| 91 | 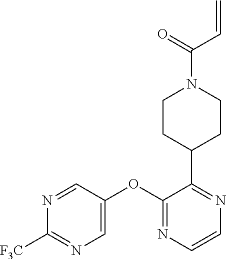 [SMILES: C=CC(=O)N1CCC(c2nccnc2Oc2cnc(C(F)(F)F)nc2)CC1] | 0.042 | 0.075 | — | — | ¹H NMR (400 MHz, DMSO) δ 9.18 (s, 2H), 8.45 (d, J = 2.7 Hz, 1H), 8.11 (d, J = 2.7 Hz, 1H), 6.88 (dd, J = 16.7, 10.5 Hz, 1H), 6.13 (dd, J = 16.7, 2.4 Hz, 1H), 5.70 (dd, J = 10.4, 2.5 Hz, 1H), 4.59 (d, J = 13.1 Hz, 1H), 4.22 (d, J = 13.7 Hz, 1H), 3.60-3.46 (m, 1H), 3.23 (d, J = 10.9 Hz, 1H), 2.86 (t, J = 12.8 Hz, 1H), 2.03 (d, J = 13.6 Hz, 2H), 1.86- |
| | | | | | | 1.56 (m, 2H). |
| 92 | 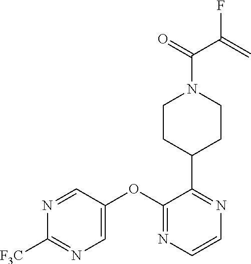 [SMILES: C=C(F)C(=O)N1CCC(c2nccnc2Oc2cnc(C(F)(F)F)nc2)CC1] | 1.12 | 5.64 | — | — | ¹H NMR (400 MHz, DMSO) δ 9.18 (s, 2H), 8.46 (d, J = 2.7 Hz, 1H), 8.11 (d, J = 2.7 Hz, 1H), 5.39-5.08 (m, 2H), 4.43 (s, 1H), 4.06 (s, 1H), 3.56 (tt, J = 11.4, 3.6 Hz, 1H), 3.32 (s, 1H), 3.00 (s, 1H), 2.20-1.98 (m, 2H), 1.78 (s, 2H). |
| 93 | 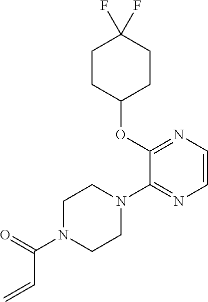 [SMILES: C=CC(=O)N1CCN(c2nccnc2OC2CCC(F)(F)CC2)CC1] | — | 0.078 | 0.007 | >20 | ¹H NMR (400 MHz, DMSO) δ 7.79 (d, J = 2.9 Hz, 1H), 7.64 (d, J = 2.9 Hz, 1H), 6.86 (dd, J = 16.7, 10.5 Hz, 1H), 6.15 (dd, J = 16.7, 2.4 Hz, 1H), 5.72 (dd, J = 10.4, 2.4 Hz, 1H), 5.29 (s, 1H), 3.76-3.57 (m, 4H), 3.46 (d, J = 9.7 Hz, 4H), 2.10-1.82 (m, 8H). |
| 94 | 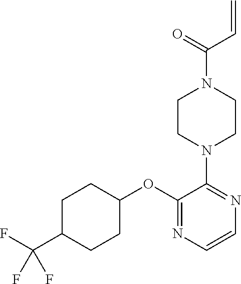 [SMILES: C=CC(=O)N1CCN(c2nccnc2OC2CCC(C(F)(F)F)CC2)CC1] | 0.017 | 0.018 | 0.00084 | — | ¹H NMR (400 MHz, DMSO) δ 7.77 (d, J = 3.0 Hz, 1H), 7.62 (d, J = 2.9 Hz, 1H), 6.84 (dd, J = 16.7, 10.4 Hz, 1H), 6.15 (d, J = 16.7 Hz, 1H), 5.72 (d, J = 10.5 Hz, 1H), 5.00 (s, 1H), 3.67 (d, J = 18.0 Hz, 4H), 3.45 (s, 4H), 2.40 (s, 1H), 2.30-2.11 (m, 2H), 2.03-1.86 (m, 2H), 1.67-1.34 (m, 4H). |
| 95 | 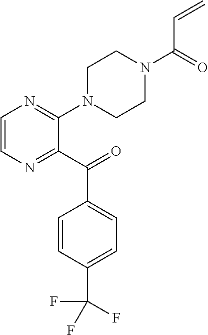 [SMILES: C=CC(=O)N1CCN(c2nccnc2C(=O)c2ccc(C(F)(F)F)cc2)CC1] | — | 0.012 | 0.0013 | — | ¹H NMR (400 MHz, DMSO) δ 8.39 (d, J = 2.3 Hz, 1H), 8.21-8.11 (m, 2H), 8.03 (d, J = 2.3 Hz, 1H), 7.94 (d, J = 8.1 Hz, 2H), 6.77 (dd, J = 16.7, 10.5 Hz, 1H), 6.14 (dd, J = 16.7, 2.4 Hz, 1H), 5.71 (dd, J = 10.5, 2.3 Hz, 1H), 3.79-3.58 (m, 4H), 3.54-3.41 (m, 4H). |
| 96 | 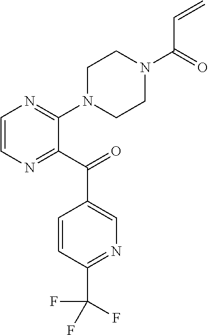 [SMILES: C=CC(=O)N1CCN(c2nccnc2C(=O)c2ccc(C(F)(F)F)nc2)CC1] | 0.089 | 0.012 | 0.0036 | — | ¹H NMR (400 MHz, DMSO) δ 9.28 (d, J = 2.0 Hz, 1H), 8.65-8.53 (m, 1H), 8.42 (d, J = 2.1 Hz, 1H), 8.12 (dd, J = 8.2, 0.9 Hz, 1H), 8.05 (d, J = 2.2 Hz, 1H), 6.79 (dd, J = 16.6, 10.4 Hz, 1H), 6.16 (dd, J = 16.7, 2.4 Hz, IH), 5.72 (dd, J = 10.4, 2.4 Hz, 1H), 3.83-3.62 (m, 4H), 3.64-3.44 (m, 4H). |
| 97 | 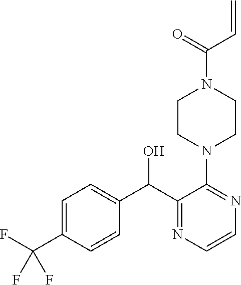 [SMILES: C=CC(=O)N1CCN(c2nccnc2C(O)c2ccc(C(F)(F)F)cc2)CC1] | — | 0.019 | 0.003 | — | ¹H NMR (400 MHz, DMSO) δ 8.28-8.19 (m, 2H), 7.68 (d, J = 8.3 Hz, 2H), 7.59 (d, J = 8.1 Hz, 2H), 6.87 (dd, J = 16.7, 10.5 Hz, 1H), 6.38 (s, 1H), 6.21- 6.10 (m, 2H), 5.72 (dd, J = 10.4, 2.4 Hz, 1H), 3.81-3.61 (m, 4H), 3.36-3.30 (m, 2H), 3.13 (s, 2H). |
| 98 | 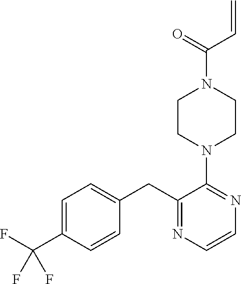 [SMILES: C=CC(=O)N1CCN(c2nccnc2Cc2ccc(C(F)(F)F)cc2)CC1] | — | 0.012 | 0.0009 | - | ¹H NMR (400 MHz, DMSO) δ 8.21 (q, J = 2.6 Hz, 2H), 7.66 (d, J = 8.0 Hz, 2H), 7.47 (d, J = 8.0 Hz, 2H), 6.86 (dd, J = 16.6, 10.4 Hz, 1H), 6.15 (dd, J = 16.7, 2.4 Hz, 1H), 5.72 (dd, J = 10.5, 2.4 Hz, 1H), 4.31 (s, 2H), 3.81- 3.63 (m, 4H), 3.12 (t, J = 5.0 Hz, 4H). |
| 99 | 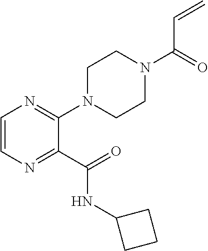 [SMILES: C=CC(=O)N1CCN(c2nccnc2C(=O)NC2CCC2)CC1] | — | 2.08 | 0.142 | — | ¹H NMR (400 MHz, DMSO) δ 8.88 (s, 1H), 8.28-8.18 (m, 1H), 7.97 (d, J = 9.4 Hz, 1H), 6.82 (d, J = 14.1 Hz, 1H), 6.14 (t, J = 11.9 Hz, 1H), 5.70 (d, J = 11.0 Hz, 1H), 4.36 (s, 1H), 3.72-3.45 (m, 8H), 2.21 (s, 2H), 2.05 (s, 2H), 1.67 (s, 2H). |
| 100 | 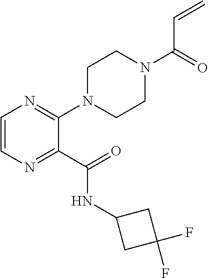 [SMILES: C=CC(=O)N1CCN(c2nccnc2C(=O)NC2CC(F)(F)C2)CC1] | — | 1.18 | 0.074 | — | ¹H NMR (400 MHz, DMSO) δ 9.19 (d, J = 6.8 Hz, 1H), 8.27 (d, J = 2.4 Hz, 1H), 8.00 (d, J = 2.3 Hz, 1H), 6.83 (dd, J = 16.7, 10.4 Hz, 1H), 6.15 (dd, J = 16.8, 2.3 Hz, 1H), 5.72 (dd, J = 10.4, 2.4 Hz, 1H), 4.31-4.13 (m, 1H), 3.72-3.58 (m, 4H), 3.45-3.43 (m, 4H), 3.32 (d, J = 3.2 Hz, 1H), 2.96 (t, J = 7.0 Hz, 2H), 2.87-2.67 (m, 2H). |
| 101 | 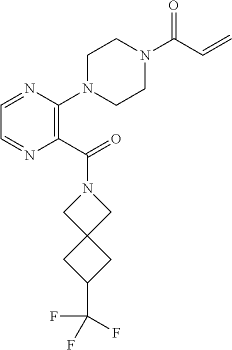 [SMILES: C=CC(=O)N1CCN(c2nccnc2C(=O)N2CC3(CC(C(F)(F)F)C3)C2)CC1] | — | 3.76 | 0.0134 | — | ¹H NMR (400 MHz, DMSO) δ 8.23 (t, J = 2.6 Hz, 1H), 7.97 (t, J = 2.7 Hz, 1H), 6.83 (dd, J = 16.7, 10.5 Hz, 1H), 6.16 (dd, J = 16.7, 2.4 Hz, 1H), 5.73 (dd, J = 10.4, 2.4 Hz, 1H), 4.25- 4.03 (m, 4H), 3.76- 3.59 (m, 4H), 3.50- 3.40 (m, 4H), 3.17- 3.00 (m, 1H), 2.51- 2.19 (m, 4H). |
| 102 | 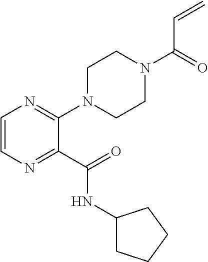 [SMILES: C=CC(=O)N1CCN(c2nccnc2C(=O)NC2CCCC2)CC1] | — | 1.6 | 0.039 | — | ¹H NMR (400 MHz, DMSO) δ 8.58 (s, 1H), 8.23 (d, J = 8.3 Hz, 1H), 7.97 (d, J = 8.1 Hz, 1H), 6.84 (s, 1H), 6.16 (d, J = 15.3 Hz, 1H), 5.71 (s, 1H), 4.16 (s, 1H), 3.74-3.54 (m, 8H), 1.88 (s, 2H), 1.67 (s, 2H), 1.53 (s, 4H). |
| 103 | 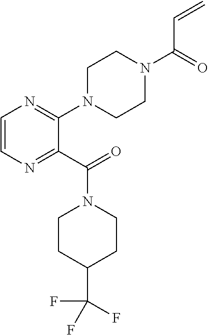 [SMILES: C=CC(=O)N1CCN(c2nccnc2C(=O)N2CCC(C(F)(F)F)CC2)CC1] | — | >20 | 0.166 | — | ¹H NMR (400 MHz, DMSO) δ 8.26 (d, J = 2.5 Hz, 1H), 8.02 (d, J = 2.5 Hz, 1H), 6.83 (dd, J = 16.7, 10.4 Hz, 1H), 6.15 (dd, J = 16.7, 2.4 Hz, 1H), 5.72 (dd, J = 10.4, 2.4 Hz, 1H), 4.60 (d, J = 13.2 Hz, 1H), 3.71-3.61 (m, 4H), 3.46 (td, J = 12.8, 6.0 Hz, 4H), 3.22-3.11 (m, 1H), 2.87 (td, J = 13.1, 2.9 Hz, 1H), 2.69 (s, 1H), 1.97 (d, J = 12.7 Hz, 1H), 1.80 (d, J = 12.9 Hz, 1H), 1.43 (pd, J = 12.8, 4.5 Hz, 2H), 1.28 (t, J = 2.3 Hz, 1H). |
| 104 | 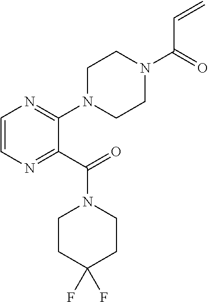 [SMILES: C=CC(=O)N1CCN(c2nccnc2C(=O)N2CCC(F)(F)CC2)CC1] | — | 2.74 | 2.22 | — | ¹H NMR (400 MHz, DMSO) δ 8.25 (s, 1H), 7.99 (s, 1H), 6.81 (d, J = 14.6 Hz, 1H), 6.16 (d, J = 14.8 Hz, 1H), 5.72 (s, 1H), 3.93-3.53 (m, 12H), 2.04 (s, 4H). |
| 105 | 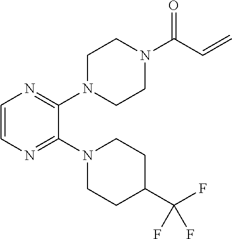 [SMILES: C=CC(=O)N1CCN(c2nccnc2N2CCC(C(F)(F)F)CC2)CC1] | 0.05 | 0.004 | 0.0011 | — | ¹H NMR (400 MHz, DMSO) δ 7.85-7.73 (m, 2H), 6.87 (dd, J = 16.6, 10.4 Hz, 1H), 6.15 (dd, J = 16.6, 2.4 Hz, 1H), 5.72 (dd, J = 10.4, 2.4 Hz, 1H), 4.17 (d, J = 12.8 Hz, 2H), 3.85-3.62 (m, 4H), 2.66 (t, J = 12.2 Hz, 2H), 1.95 (d, J = 12.6 Hz, 2H), 1.64-1.43 (m, 2H). |
| 106 | 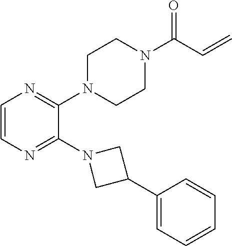 [SMILES: C=CC(=O)N1CCN(c2nccnc2N2CC(c3ccccc3)C2)CC1] | — | 20 | 0.007 | >20 | ¹H NMR (400 MHz, DMSO) δ 7.81 (d, J = 2.8 Hz, 1H), 7.69 (t, J = 2.5 Hz, 1H), 7.47- 7.21 (m, 6H), 6.90- 6.77 (m, 1H), 6.14 (ddd, J = 16.9, 4.0, 2.5 Hz, 1H), 5.72 (dd, J = 10.4, 2.4 Hz, 1H), 4.43 (t, J = 8.2 Hz, 2H), 4.04- 3.87 (m, 3H), 3.72 (d, J = 14.7 Hz, 4H), 3.63 (d, J = 6.7 Hz, 1H), 3.14 (s, 4H). |
| 107 | 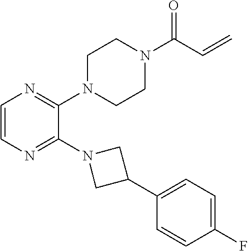 [SMILES: C=CC(=O)N1CCN(c2nccnc2N2CC(c3ccc(F)cc3)C2)CC1] | 0.049 | 0.018 | 0.0007 | — | ¹H NMR (400 MHz, DMSO) δ 7.81 (d, J = 2.8 Hz, 1H), 7.69 (d, J = 2.8 Hz, 1H), 7.53- 7.39 (m, 2H), 7.25- 7.10 (m, 2H), 6.85 (dd, J = 16.7, 10.5 Hz, 1H), 6.14 (dd, J = 16.7, 2.4 Hz, 1H), 5.72 (dd, J = 10.4, 2.4 Hz, 1H), 4.49- 4.35 (m, 2H), 4.04- 3.86 (m, 3H), 3.72 (d, J = 13.9 Hz, 4H), 3.14 (s, 4H). |
| 108 | 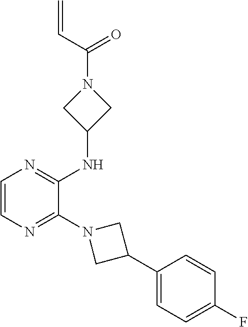 [SMILES: C=CC(=O)N1CC(Nc2nccnc2N2CC(c3ccc(F)cc3)C2)C1] | — | >10 | — | — | ¹H NMR (400 MHz, DMSO) δ 7.48 (q, J = 3.9 Hz, 4H), 7.19 (t, J = 8.8 Hz, 2H), 6.43 (d, J = 6.3 Hz, 1H), 6.37- 6.26 (m, 1H), 6.10 (d, J = 17.2 Hz, 1H), 5.67 (dd, J = 10.0, 2.2 Hz, 1H), 4.67-4.42 (m, 4H), 4.27-4.16 (m, 1H), 4.10-3.98 (m, 3H), 3.93-3.83 (m, 2H). |
| 109 | 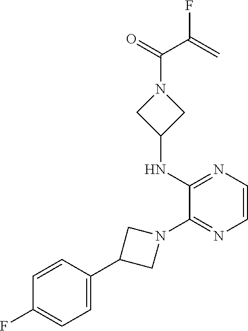 [SMILES: C=C(F)C(=O)N1CC(Nc2nccnc2N2CC(c3ccc(F)cc3)C2)C1] | — | >10 | — | — | ¹H NMR (400 MHz, DMSO) δ 7.48 (q, J = 4.8 Hz, 4H), 7.19 (t, J = 8.7 Hz, 2H), 6.43 (d, J = 5.5 Hz, 1H), 5.56- 5.23 (m, 2H), 4.63 (s, 2H), 4.47 (q, J = 8.8 Hz, 2H), 4.25 (d, J = 7.3 Hz, 2H), 4.04 (q, J = 8.0 Hz, 2H), 3.90 (q, J = 7.7 Hz, 2H). |
| 110 | 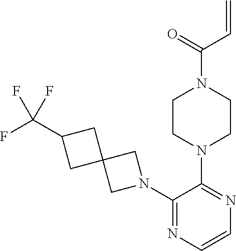 [SMILES: C=CC(=O)N1CCN(c2nccnc2N2CC3(CC(C(F)(F)F)C3)C2)CC1] | 0.046 | 0.067 | 0.003 | — | ¹H NMR (400 MHz, DMSO) δ 7.76 (d, J = 2.8 Hz, 1H), 7.64 (d, J = 2.8 Hz, IH), 6.85 (dd, J = 16.7, 10.5 Hz, 1H), 6.15 (dd, J = 16.7, 2.4 Hz, 1H), 5.72 (dd, J = 10.5, 2.4 Hz, 1H), 4.04 (s, 2H), 3.96 (s, 2H), 3.72 (d, J = 13.5 Hz, 4H), 3.09 (s, 5H), 2.46 (dd, J = 13.1, 9.1 Hz, 2H), 2.30 (dd, J = 13.1, 7.4 Hz, 2H). |
| 111 | 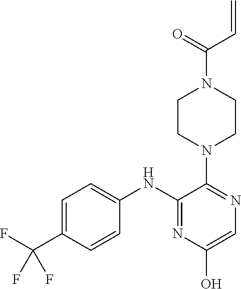 [SMILES: C=CC(=O)N1CCN(c2ncc(O)nc2Nc2ccc(C(F)(F)F)cc2)CC1] | — | 5.64 | 0.239 | — | ¹H NMR (400 MHz, DMSO) δ 8.48 (s, 1H), 8.02 (d, J = 8.7 Hz, 2H), 7.64 (d, J = 8.5 Hz, 2H), 7.31 (s, 1H), 6.86 (d, J = 6.6 Hz, 1H), 6.15 (d, J = 16.7 Hz, 1H), 5.74-5.69 (m, 1H), 2.93 (s, 4H). |
| 112 | 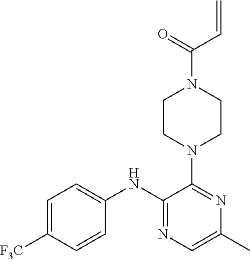 [SMILES: C=CC(=O)N1CCN(c2nc(C)cnc2Nc2ccc(C(F)(F)F)cc2)CC1] | 0.009 | 0.011 | 0.0018 | 10 | ¹H NMR (400 MHz, DMSO) δ 8.52 (s, 1H), 7.88-7.74 (m, 3H), 7.62 (d, J = 8.5 Hz, 2H), 6.85 (dd, J = 16.7, 10.5 Hz, 1H), 6.15 (dd, J = 16.7, 2.4 Hz, 1H), 5.72 (dd, J = 10.4, 2.4 Hz, 1H), 3.77 (d, J = 13.6 Hz, 4H), 3.14 (s, 4H), 2.33 (s, 3H). |
| 113 | 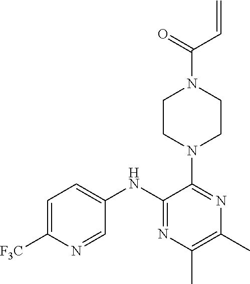 [SMILES: C=CC(=O)N1CCN(c2nc(C)c(C)nc2Nc2ccc(C(F)(F)F)nc2)CC1] | 0.032 | 0.040 | 0.0027 | — | ¹H NMR (400 MHz, DMSO) δ 9.04 (d, J = 2.5 Hz, 1H), 8.71 (s, 1H), 8.40 - 8.26 (m, 1H), 7.79 (d, J = 8.7 Hz, 1H), 6.85 (dd, J = 16.7, 10.4 Hz, 1H), 6.15 (dd, J = 16.8, 2.4 Hz, 1H), 5.72 (dd, J = 10.5, 2.4 Hz, 1H), 3.76 (d, J = 13.1 Hz, 4H), 3.08 (s, 4H), 2.35 (d, J = 5.2 Hz, 6H). |
| 114 | 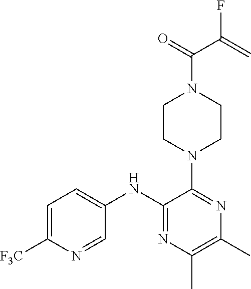 [SMILES: C=C(F)C(=O)N1CCN(c2nc(C)c(C)nc2Nc2ccc(C(F)(F)F)nc2)CC1] | — | 0.072 | 0.0044 | — | ¹H NMR (400 MHz, DMSO) δ 9.04 (s, 1H), 8.71 (s, 1H), 8.34 (d, J = 8.4 Hz, 1H), 7.79 (d, J = 8.7 Hz, 1H), 5.38- 5.12 (m, 2H), 3.74 (d, J = 5.7 Hz, 4H), 3.12 (s, 4H), 2.35 (d, J = 5.7 Hz, 6H). |
| 115 | 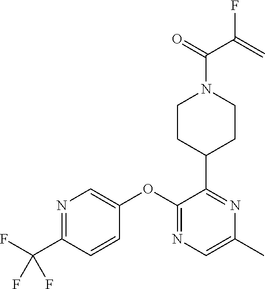 [SMILES: C=C(F)C(=O)N1CCC(c2nc(C)cnc2Oc2ccc(C(F)(F)F)nc2)CC1] | 0.041 | 0.074 | — | — | ¹H NMR (400 MHz, DMSO) δ 8.73 (d, J = 2.5 Hz, 1H), 8.09- 7.90 (m, 3H), 5.33- 5.10 (m, 2H), 4.41 (s, 1H), 4.04 (s, 1H), 3.52- 3.46 (m, 2H), 2.97 (s, 1H), 2.46 (s, 3H), 2.01 (d, J = 13.2 Hz, 2H), 1.77 (s, 2H). |
| 116 | 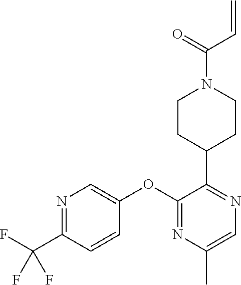 [SMILES: C=CC(=O)N1CCC(c2ncc(C)nc2Oc2ccc(C(F)(F)F)nc2)CC1] | — | 0.022 | — | — | ¹H NMR (400 MHz, DMSO) δ 8.76 (d, J = 2.4 Hz, 1H), 8.30 (s, 1H), 8.08-7.91 (m, 2H), 6.94-6.80 (m, 1H), 6.12 (ddd, J = 16.7, 2.4, 1.0 Hz, 1H), 5.69 (ddd, J = 10.4, 2.5, 1.0 Hz, 1H), 4.56 (d, J = 13.0 Hz, 1H), 4.20 (d, J = 13.7 Hz, 1H), 3.33- 3.19 (m, 2H), 2.83 (t, J = 12.6 Hz, 1H), 2.31 (s, 3H), 1.97 (d, J = 13.1 Hz, 2H), 1.84- 1.55 (m, 2H). |
| 117 | 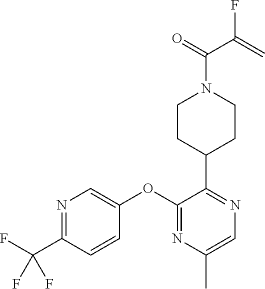 [SMILES: C=C(F)C(=O)N1CCC(c2ncc(C)nc2Oc2ccc(C(F)(F)F)nc2)CC1] | — | 0.158 | — | — | ¹H NMR (400 MHz, DMSO) δ 8.76 (d, J = 2.3 Hz, 1H), 8.31 (s, 1H), 8.08-7.94 (m, 2H), 5.33-5.07 (m, 2H), 4.40 (s, 1H), 4.03 (s, 1H), 3.54-3.45 (m, 2H), 3.06-2.87 (m, 1H), 2.31 (s, 3H), 2.12- 1.95 (m, 2H), 1.75 (s, 2H). |
| 118 | 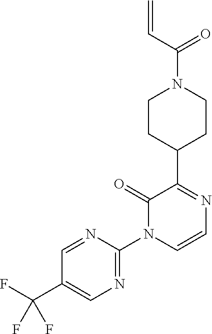 [SMILES: C=CC(=O)N1CCC(c2nccn(-c3ncc(C(F)(F)F)cn3)c2=O)CC1] | — | >10 | — | — | ¹H NMR (400 MHz, DMSO-d6) δ 9.54 (s, 2H), 7.73 (d, J = 4.6 Hz, 1H), 7.38 (d, J = 4.6 Hz, 1H), 6.83 (dd, J = 16.7, 10.5 Hz, 1H), 6.10 (dd, J = 16.7, 2.4 Hz, 1H), 5.67 (dd, J = 10.5, 2.5 Hz, 1H), 4.50 (d, J = 12.7 Hz, 1H), 4.15 (m, 1H), 3.40 (m, 1H), 3.19 (m, 1H), 2.79 (m, 1H), 1.91 (m, 2H), 1.52 (m, 2H). |
| 119 | 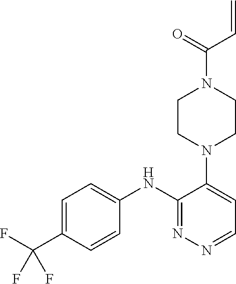 [SMILES: C=CC(=O)N1CCN(c2ccnnc2Nc2ccc(C(F)(F)F)cc2)CC1] | 0.042 | 0.012 | 0.0032 | — | ¹H NMR (400 MHz, DMSO) δ 8.68 (d, J = 5.2 Hz, 1H), 8.55 (s, 1H), 7.88 (d, J = 8.5 Hz, 2H), 7.66 (d, J = 8.5 Hz, 2H), 7.15 (d, J = 5.3 Hz, 1H), 6.85 (dd, J = 16.7, 10.5 Hz, 1H), 6.16 (dd, J = 16.7, 2.4 Hz, 1H), 5.73 (dd, J = 10.4, 2.4 Hz, 1H), 3.77 (d, J = 13.8 Hz, 4H), 3.04 (t, J = 4.9 Hz, 4H). |
| 120 | 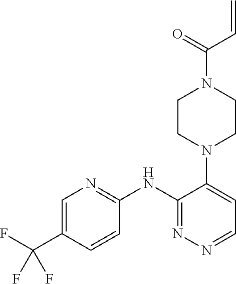 [SMILES: C=CC(=O)N1CCN(c2ccnnc2Nc2ccc(C(F)(F)F)cn2)CC1] | 0.205 | — | — | — | ¹H NMR (400 MHz, DMSO) δ 9.12 (s, 1H), 8.78 (d, J = 5.4 Hz, 1H), 8.61 (s, 1H), 8.10 (d, J = 8.9 Hz, 1H), 7.96 (d, J = 8.9 Hz, 1H), 7.22 (d, J = 5.4 Hz, 1H), 6.84 (dd, J = 16.7, 10.4 Hz, 1H), 6.15 (d, J = 17.0 Hz, 1H), 5.77-5.66 (m, 1H), 3.68 (d, J = 15.1 Hz, 4H), 3.09 (d, J = 5.6 Hz, 4H). |
| 121 | 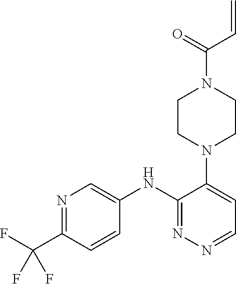 [SMILES: C=CC(=O)N1CCN(c2ccnnc2Nc2ccc(C(F)(F)F)nc2)CC1] | 1.4 | — | — | — | ¹H NMR (400 MHz, DMSO) δ 8.99 (s, 1H), 8.79 (s, 1H), 8.72 (d, J = 5.2 Hz, 1H), 8.42- 8.32 (m, 1H), 7.85 (d, J = 8.6 Hz, 1H), 7.18 (d, J = 5.4 Hz, 1H), 6.86 (dd, J = 16.7, 10.4 Hz, 1H), 6.16 (d, J = 16.5 Hz, 1H), 5.73 (d, J = 10.5 Hz, 1H), 3.78 (s, 4H), 3.06 (s, 4H). |
| 122 | 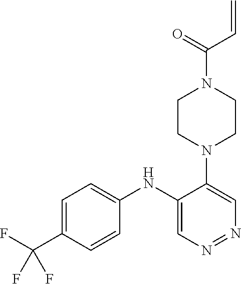 [SMILES: C=CC(=O)N1CCN(c2cnncc2Nc2ccc(C(F)(F)F)cc2)CC1] | — | >10 | 0.0068 | — | ¹H NMR (400 MHz, DMSO) δ 8.85 (s, 1H), 8.79 (d, J = 2.6 Hz, 2H), 7.63 (d, J = 8.4 Hz, 2H), 7.11 (d, J = 8.4 Hz, 2H), 6.79 (dd, J = 16.6, 10.4 Hz, 1H), 6.12 (dd, J = 16.7, 2.4 Hz, 1H), 5.69 (dd, J = 10.4, 2.4 Hz, 1H), 3.44 (d, J = 14.7 Hz, 4H), 3.08 (t, J = 5.1 Hz, 4H). |
| 123 | 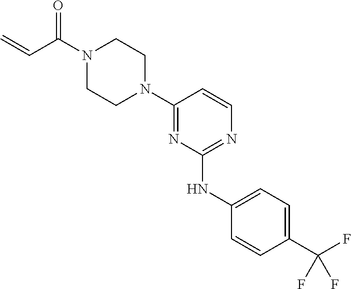 [SMILES: C=CC(=O)N1CCN(c2ccnc(Nc3ccc(C(F)(F)F)cc3)n2)CC1] | — | 0.910 | 0.2787 | 4.87 | ¹H NMR (400 MHz, DMSO) δ 9.56 (s, IH), 8.05 (d, J = 6.1 Hz, 1H), 7.93 (d, J = 8.6 Hz, 2H), 7.59 (d, J = 8.7 Hz, 2H), 6.85 (dd, J = 16.6, 10.4 Hz, 1H), 6.38 (d, J = 6.1 Hz, 1H), 6.16 (dd, J = 16.7, 2.3 Hz, 1H), 5.73 (dd, J = 10.4, 2.3 Hz, 1H), 3.67 (d, J = 14.2 Hz, 8H). |
| 124 | 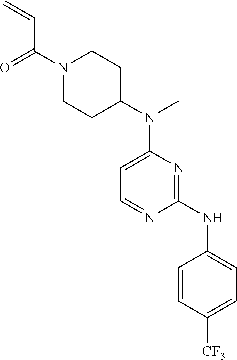 [SMILES: C=CC(=O)N1CCC(N(C)c2ccnc(Nc3ccc(C(F)(F)F)cc3)n2)CC1] | — | 4.64 | 0.331 | 4.2 | ¹H NMR (400 MHz, DMSO) δ 10.21 (s, 1H), 8.14-7.92 (m, 1H), 7.94-7.32 (m, 4H), 6.93-6.70 (m, 1H), 6.25-6.02 (m, 1H), 5.68 (ddd, J = 10.5, 8.0, 2.5 Hz, 1H), 4.94-4.35 (m, 2H), 4.34-3.96 (m, 1H), 3.26-3.05 (m, 1H), 3.07-2.78 (m, 3H), 2.72 (t, J = 12.9 Hz, 1H), 1.71 (s, 4H). |
| 125 | 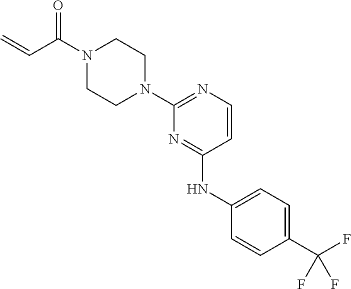 [SMILES: C=CC(=O)N1CCN(c2nccc(Nc3ccc(C(F)(F)F)cc3)n2)CC1] | — | 2.52 | 0.2583 | 7.06 | ¹H NMR (400 MHz, DMSO) δ 9.71 (s, 1H), 8.03 (d, J = 5.6 Hz, 1H), 7.86 (d, J = 8.6 Hz, 2H), 7.66 (d, J = 8.5 Hz, 2H), 6.84 (dd, J = 16.7, 10.4 Hz, 1H), 6.22-6.03 (m, 2H), 5.72 (dd, J = 10.5, 2.4 Hz, 1H), 3.74 (s, 4H), 3.65 (d, J = 15.5 Hz, 4H). |
| 126 | 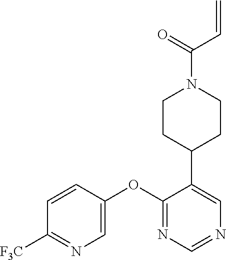 [SMILES: C=CC(=O)N1CCC(c2cncnc2Oc2ccc(C(F)(F)F)nc2)CC1] | 0.339 | 0.364 | 0.0191 | — | ¹H NMR (400 MHz, DMSO) δ 8.90-8.55 (m, 3H), 8.08 (d, J = 1.6 Hz, 2H), 6.87 (dd, J = 16.7, 10.5 Hz, 1H), 6.13 (dd, J = 16.7, 2.5 Hz, 1H), 5.70 (dd, J = 10.5, 2.5 Hz, 1H), 4.64 (d, J = 13.1 Hz, 1H), 4.24 (d, J = 13.8 Hz, 1H), 3.29-3.12 (m, 2H), 2.78 (t, J = 12.7 Hz, 1H), 2.01 (d, J = 12.8 Hz, 2H), 1.88- 1.60 (m, 2H). |
| 127 | 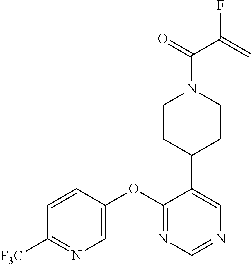 [SMILES: C=C(F)C(=O)N1CCC(c2cncnc2Oc2ccc(C(F)(F)F)nc2)CC1] | — | 66.81 | — | — | ¹H NMR (400 MHz, DMSO) δ 8.79 (s, 1H), 8.73 (s, 1H), 8.65 (s, 1H), 8.08 (d, J = 1.6 Hz, 2H), 5.36-5.10 (m, 2H), 4.47 (s, 1H), 4.07 (s, 1H), 3.29 (ddd, J = 12.6, 8.7, 3.6 Hz, 2H), 2.92 (s, 1H), 2.13- 1.98 (m, 2H), 1.91- 1.69 (m, 2H). |
| 128 | 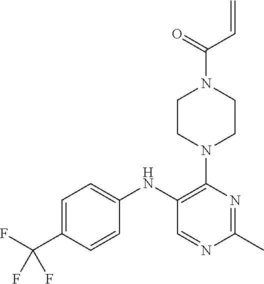 [SMILES: C=CC(=O)N1CCN(c2nc(C)ncc2Nc2ccc(C(F)(F)F)cc2)CC1] | 0.082 | 0.179 | 0.0016 | — | ¹H NMR (400 MHz, DMSO) δ 8.30 (s, 1H), 8.08 (s, 1H), 7.48 (d, J = 8.5 Hz, 2H), 6.77 (dd, J = 16.7, 10.4 Hz, 1H), 6.69 (d, J = 8.5 Hz, 2H), 6.10 (dd, J = 16.7, 2.4 Hz, 1H), 5.67 (dd, J = 10.4, 2.4 Hz, 1H), 3.59 (t, J = 4.8 Hz, 4H), 3.52-3.39 (m, 4H), 2.46 (s, 3H). |
| 129 | 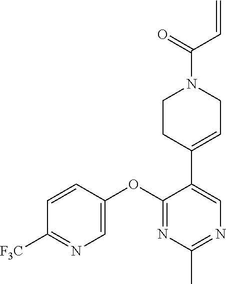 [SMILES: C=CC(=O)N1CC=C(c2cnc(C)nc2Oc2ccc(C(F)(F)F)nc2)CC1] | 0.137 | 0.104 | 0.0012 | — | ¹H NMR (400 MHz, DMSO) δ 8.78 (d, J = 1.7 Hz, 1H), 8.60 (d, J = 2.2 Hz, 1H), 8.06 (d, J = 1.6 Hz, 2H), 6.95- 6.75 (m, 1H), 6.27 (s, 1H), 6.16 (d, J = 16.4 Hz, 1H), 5.72 (d, J = 10.5 Hz, 1H), 4.38- 4.17 (m, 2H), 3.77 (dt, J = 12.0, 5.7 Hz, 2H), 2.71-2.55 (m, 2H), 2.42 (s, 3H). |
| 130 | 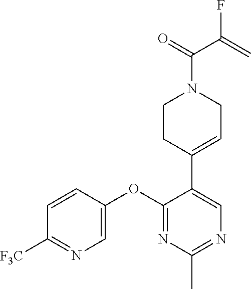 [SMILES: C=C(F)C(=O)N1CC=C(c2cnc(C)nc2Oc2ccc(C(F)(F)F)nc2)CC1] | — | 1.33 | 0.0011 | — | ¹H NMR (400 MHz, DMSO) δ 8.78 (t, J = 1.7 Hz, 1H), 8.61 (s, 1H), 8.06 (d, J = 1.6 Hz, 2H), 6.27 (s, 1H), 5.40-5.11 (m, 2H), 4.35-4.11 (m, 2H), 3.75 (t, J = 5.6 Hz, 2H), 2.66 (s, 2H), 2.43 (s, 3H) |
| 131 | 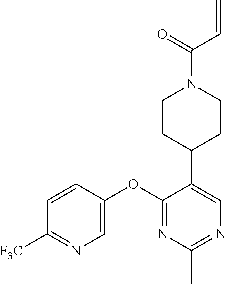 [SMILES: C=CC(=O)N1CCC(c2cnc(C)nc2Oc2ccc(C(F)(F)F)nc2)CC1] | — | 2.25 | 0.598 | — | ¹H NMR (400 MHz, DMSO) δ 8.80-8.56 (m, 1H), 8.35-8.04 (m, 2H), 7.82-7.68 (m, 2H), 7.36 (dd, J = 8.5, 2.8 Hz, 1H), 6.91- 6.74 (m, 1H), 6.12 (ddd, J = 16.7, 5.3, 2.5 Hz, 1H), 5.69 (ddd, J = 10.4, 4.4, 2.4 Hz, 1H), 4.57 (d, J = 13.1 Hz, 1H), 4.27-4.16 (m, 1H), 3.22-3.06 (m, 2H), 2.84 (t, J = 12.4 Hz, 1H), 2.42-2.31 (m, 4H), 1.96 (s, 2H), 1.49 (d, J = 14.0 Hz, 2H). |
| 132 | 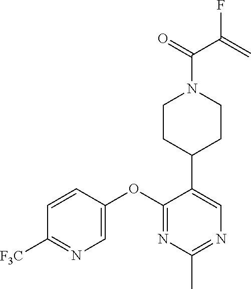 [SMILES: C=C(F)C(=O)N1CCC(c2cnc(C)nc2Oc2ccc(C(F)(F)F)nc2)CC1] | — | 4.31 | 0.451 | — | ¹H NMR (400 MHz, DMSO) δ 8.82-8.58 (m, 1H), 8.32-7.99 (m, 2H), 7.81-7.34 (m, 1H), 5.35-5.08 (m, 2H), 4.45 (s, 1H), 3.23 (t, J = 12.3 Hz, 2H), 2.88 (s, 1H), 2.43- 2.31 (m, 3H), 2.01 (d, J = 12.8 Hz, 2H), 1.86- 1.66 (m, 2H). |
| 133 | 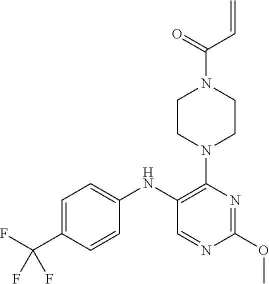 [SMILES: C=CC(=O)N1CCN(c2nc(OC)ncc2Nc2ccc(C(F)(F)F)cc2)CC1] | — | 0.722 | 0.0165 | — | ¹H NMR (400 MHz, DMSO) δ 8.12 (s, 1H), 7.95 (s, 1H), 7.47 (d, J = 8.5 Hz, 2H), 6.77 (dd, J = 16.6, 10.5 Hz, 1H), 6.64 (d, J = 8.5 Hz, 2H), 6.10 (dd, J = 16.7, 2.4 Hz, 1H), 5.67 (dd, J = 10.4, 2.4 Hz, 1H), 3.86 (s, 3H), 3.68 (s, 4H), 3.55-3.41 (m, 4H). |
| 134 | 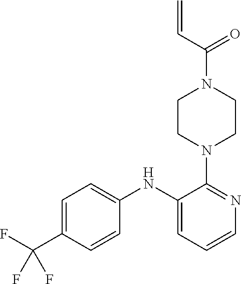 [SMILES: C=CC(=O)N1CCN(c2ncccc2Nc2ccc(C(F)(F)F)cc2)CC1] | — | 0.023 | 0.002238 | >10 | ¹H NMR (400 MHz, DMSO) δ 8.15 (s, 1H), 8.02 (dd, J = 4.7, 1.7 Hz, 1H), 7.56 (dd, J = 7.8, 1.7 Hz, 1H), 7.50 (d, J = 8.5 Hz, 2H), 7.12-6.91 (m, 3H), 6.78 (dd, J = 16.7, 10.5 Hz, 1H), 6.10 (dd, J = 16.7, 2.4 Hz, 1H), 5.66 (dd, J = 10.4, 2.4 Hz, 1H), 3.52 (d, J = 12.6 Hz, 4H), 3.14 (t, J = 5.2 Hz, 4H). |
| 135 | 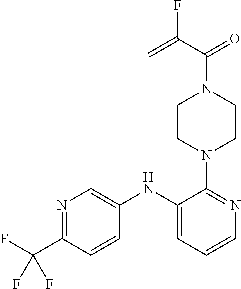 [SMILES: C=C(F)C(=O)N1CCN(c2ncccc2Nc2ccc(C(F)(F)F)nc2)CC1] | — | 1.67 | 0.41 | — | ¹H NMR (400 MHz, DMSO) δ 8.53 (s, 1H), 8.31 (d, J = 2.7 Hz, 1H), 8.09 (dd, J = 4.8, 1.6 Hz, 1H), 7.71- 7.56 (m, 2H), 7.24 (dd, J = 8.6, 2.7 Hz, 1H), 7.04 (dd, J = 7.8, 4.7 Hz, 1H), 5.31-5.05 (m, 2H), 3.47 (t, J = 5.0 Hz, 4H), 3.19 (d, J = 5.3 Hz, 4H). |
| 136 | 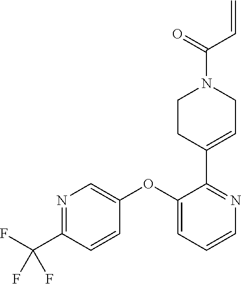 [SMILES: C=CC(=O)N1CC=C(c2ncccc2Oc2ccc(C(F)(F)F)nc2)CC1] | — | 0.341 | 0.066 | — | ¹H NMR (400 MHz, DMSO) δ 8.52 (d, J = 2.8 Hz, 1H), 8.26- 8.16 (m, 2H), 8.09 (dd, J = 4.9, 1.8 Hz, 1H), 7.50 (dd, J = 7.4, 1.9 Hz, 1H), 7.22 (d, J = 8.8 Hz, 1H), 6.91 (dd, J = 7.4, 4.9 Hz, 1H), 5.91 (s, 1H), 5.41-5.13 (m, 2H), 4.31-4.08 (m, 2H), 3.76 (d, J = 5.7 Hz, 2H), 3.41 (d, J = 1.0 Hz, 2H), 2.47 (s, 2H). |
| 137 | 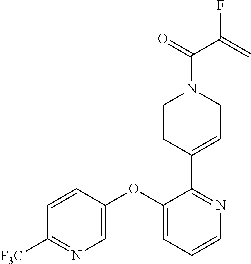 [SMILES: C=C(F)C(=O)N1CC=C(c2ncccc2Oc2ccc(C(F)(F)F)nc2)CC1] | — | 0.243 | 0.022 | — | ¹H NMR (400 MHz, DMSO) δ 8.54 (dd, J = 13.7, 3.8 Hz, 2H), 7.89 (d, J = 8.8 Hz, 1H), 7.71 (dd, J = 15.8, 8.3 Hz, 1H), 7.53 (s, 1H), 7.43 (dd, J = 8.3, 4.5 Hz, 1H), 6.46 (s, 1H), 5.36-5.06 (m, 2H), 4.10 (s, 2H), 3.63 (t, J = 5.7 Hz, 2H), 2.68 (s, 2H). |
| 138 | 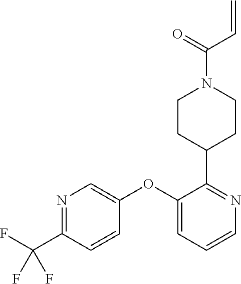 [SMILES: C=CC(=O)N1CCC(c2ncccc2Oc2ccc(C(F)(F)F)nc2)CC1] | 0.134 | 2.4 | 0.054 | — | ¹H NMR (400 MHz, DMSO) δ 8.63 (d, J = 2.8 Hz, 1H), 8.47 (dd, J = 4.6, 1.4 Hz, 1H), 7.92 (d, J = 8.7 Hz, 1H), 7.58 (td, J = 9.1, 2.1 Hz, 2H), 7.37 (dd, J = 8.2, 4.6 Hz, 1H), 6.84 (dd, J = 16.7, 10.4 Hz, 1H), 6.11 (dd, J = 16.7, 2.5 Hz, 1H), 5.67 (dd, J = 10.5, 2.4 Hz, 1H), 4.52 (d, J = 13.2 Hz, 1H), 4.14 (d, J = 13.7 Hz, 1H), 3.32-3.27 (m, 1H), 3.13 (d, J = 15.4 Hz, 1H), 2.78- 2.68 (m, 1H), 1.86- |
| | | | | | | 1.64 (m, 4H). |
| 139 | 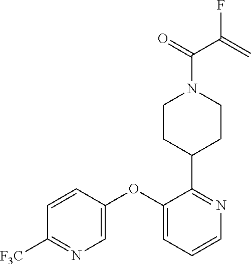 [SMILES: C=C(F)C(=O)N1CCC(c2ncccc2Oc2ccc(C(F)(F)F)nc2)CC1] | — | 7.03 | 3.24 | — | ¹H NMR (400 MHz, DMSO) δ 8.63 (d, J = 2.8 Hz, 1H), 8.55- 8.40 (m, 1H), 7.92 (d, J = 8.8 Hz, 1H), 7.62- 7.49 (m, 2H), 7.37 (dd, J = 8.2, 4.6 Hz, 1H), 5.33-5.09 (m, 2H), 4.36 (s, 1H), 3.97 (s, 1H), 3.27 (s, 1H), 3.10- 2.78 (m, 2H), 1.92- 1.67 (m, 4H). |
| 140 | 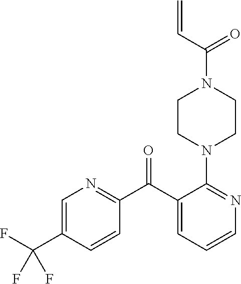 [SMILES: C=CC(=O)N1CCN(c2ncccc2C(=O)c2ccc(C(F)(F)F)cn2)CC1] | — | 11.09 | 0.0086 | — | ¹H NMR (400 MHz, DMSO) δ 9.08 (d, J = 2.2 Hz, 1H), 8.51 (dd, J = 8.3, 2.3 Hz, 1H), 8.40 (dd, J = 4.7, 1.9 Hz, 1H), 8.22 (d, J = 8.2 Hz, 1H), 7.85 (dd, J = 7.6, 1.9 Hz, 1H), 6.99 (dd, J = 7.6, 4.7 Hz, 1H), 6.72 (dd, J = 16.7, 10.4 Hz., 1H), 6.09 (dd, J = 16.7, 2.4 Hz, 1H), 5.67 (dd, J = 10.4, 2.4 Hz, 1H), 3.39 (s, 4H), 3.22-3.15 (m, 4H). |
| 141 | 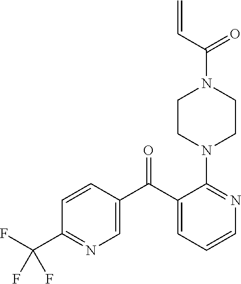 [SMILES: C=CC(=O)N1CCN(c2ncccc2C(=O)c2ccc(C(F)(F)F)nc2)CC1] | — | 0.5 | 0.0075 | — | ¹H NMR (400 MHz, DMSO) δ 9.06 (d, J = 2.1 Hz, 1H), 8.46 (dd, J = 4.7, 1.9 Hz, 1H), 8.39 (dd, J = 8.2, 2.1 Hz, 1H), 8.10 (d, J = 8.1 Hz, 1H), 7.88 (dd, J = 7.6, 1.9 Hz, 1H), 7.05 (dd, J = 7.6, 4.7 Hz, 1H), 6.72 (dd, J = 16.7, 10.5 Hz, 1H), 6.09 (dd, J = 16.7, 2.3 Hz, 1H), 5.67 (dd, J = 10.4, 2.4 Hz, 1H), 3.34-3.26 (m, 4H), 3.21 (s, 4H). |
| 142 | 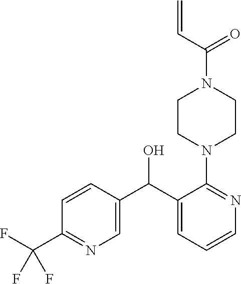 [SMILES: C=CC(=O)N1CCN(c2ncccc2C(O)c2ccc(C(F)(F)F)nc2)CC1] | — | 7.28 | 0.0023 | — | ¹H NMR (400 MHz, DMSO) δ 8.78 (s, 1H), 8.28 (dd, J = 4.8, 1.9 Hz, 1H), 7.98 (d, J = 8.1 Hz, 1H), 7.91- 7.78 (m, 2H), 7.19 (dd, J = 7.6, 4.7 Hz, 1H), 6.85 (dd, J = 16.7, 10.4 Hz, 1H), 6.38 (s, 1H), 6.27-6.09 (m, 2H), 5.71 (dd, J = 10.5, 2.4 Hz, 1H), 3.86-3.54 (m, 4H), 3.20-3.06 (m, 2H), 2.80 (s, 2H). |
| 143 | 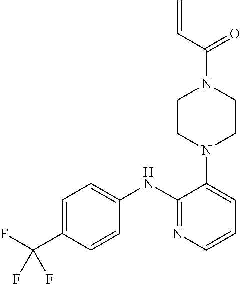 [SMILES: C=CC(=O)N1CCN(c2cccnc2Nc2ccc(C(F)(F)F)cc2)CC1] | — | 0.017 | 0.005 | 20 | ¹H NMR (400 MHz, DMSO) δ 8.43 (s, 1H), 8.10-7.92 (m, 3H), 7.61 (d, J = 8.6 Hz, 2H), 7.50 (dd, J = 7.7, 1.6 Hz, 1H), 6.98- 6.75 (m, 2H), 6.15 (dd, J = 16.7, 2.5 Hz, 1H), 5.71 (dd, J = 10.4, 2.4 Hz, 1H), 3.82 (s, 4H), 2.85 (t, J = 4.9 Hz, 4H). |
| 144 | 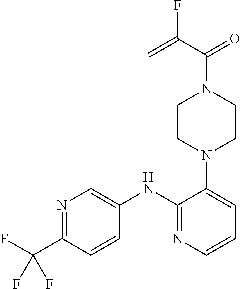 [SMILES: C=C(F)C(=O)N1CCN(c2cccnc2Nc2ccc(C(F)(F)F)nc2)CC1] | — | 0.031 | 0.0081 | — | ¹H NMR (400 MHz, DMSO) δ 9.07 (d, J = 8.1 Hz, 1H), 8.69- 8.45 (m, 2H), 8.02 (d, J = 8.5 Hz, 1H), 7.80 (s, 1H), 7.55 (s, 1H), 6.96 (d, J = 8.6 Hz, 1H), 5.40-5.12 (m, 2H), 3.80 (s, 4H), 2.91 (d, J = 9.4 Hz, 4H). |
| 145 | 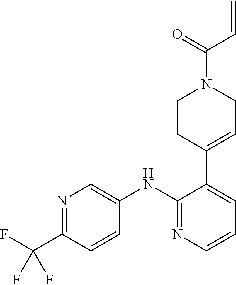 [SMILES: C=CC(=O)N1CC=C(c2cccnc2Nc2ccc(C(F)(F)F)nc2)CC1] | 0.017 | 0.029 | 0.0024 | — | ¹H NMR (400 MHz, DMSO) δ 8.91 (d, J = 2.5 Hz, 1H), 8.55 (d, J = 7.8 Hz, 1H), 8.28 (d, J = 8.9 Hz, 1H), 8.19 (dd, J = 4.9, 1.8 Hz, 1H), 7.76 (d, J = 8.7 Hz, 1H), 7.57 (d, J = 7.5 Hz, 1H), 7.01 (dd, J = 7.5, 4.9 Hz, 1H), 6.94-6.77 (m, 1H), 6.24- 6.11 (m, 1H), 5.95 (d, J = 10.5 Hz, 1H), 5.82- 5.69 (m, 1H), 4.38- 4.10 (m, 2H), 3.80 (d, J = 6.1 Hz, 2H), 2.46- 2.33 (m, 2H). |
| 146 | 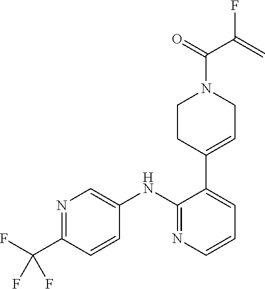 [SMILES: C=C(F)C(=O)N1CC=C(c2cccnc2Nc2ccc(C(F)(F)F)nc2)CC1] | — | 0.158 | 0.0235 | — | ¹H NMR (400 MHz, DMSO) δ 8.91 (d, J = 2.6 Hz, 1H), 8.56 (s, 1H), 8.32-8.13 (m, 2H), 7.76 (d, J = 8.7 Hz, 1H), 7.58 (dd, J = 7.5, 1.9 Hz, 1H), 7.01 (dd, J = 7.5, 4.9 Hz, 1H), 5.95 (s, 1H), 5.43- 5.12 (m, 2H), 4.34- 4.13 (m, 2H), 3.78 (s, 2H), 3.30-3.17 (m, 2H). |
| 147 | 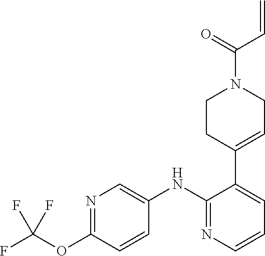 [SMILES: C=CC(=O)N1CC=C(c2cccnc2Nc2ccc(OC(F)(F)F)nc2)CC1] | 0.149 | — | 0.0023 | — | ¹H NMR (400 MHz, DMSO) δ 8.55 (d, J = 2.8 Hz, 1H), 8.21 (dd, J = 9.3, 3.7 Hz, 2H), 8.10 (dd, J = 4.8, 1.9 Hz, 1H), 7.57-7.43 (m, 1H), 7.23 (d, J = 8.8 Hz, 1H), 7.00-6.75 (m, 2H), 6.24-6.08 (m, 1H), 5.93 (d, J = 9.4 Hz, 1H), 5.81- 5.67 (m, 1H), 4.44- 4.12 (m, 2H), 3.81 (d, J = 6.0 Hz, 2H), 2.47- 2.30 (m, 2H). |
| 148 | 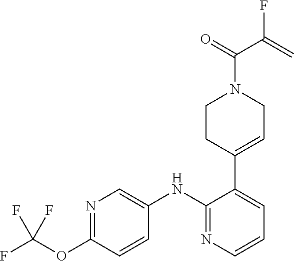 [SMILES: C=C(F)C(=O)N1CC=C(c2cccnc2Nc2ccc(OC(F)(F)F)nc2)CC1] | — | 0.341 | 0.066 | — | ¹H NMR (400 MHz, DMSO) δ 8.52 (d, J = 2.8 Hz, 1H), 8.26- 8.16 (m, 2H), 8.09 (dd, J = 4.9, 1.8 Hz, 1H), 7.50 (dd, J = 7.4, 1.9 Hz, 1H), 7.22 (d, J = 8.8 Hz, 1H), 6.91 (dd, J = 7.4, 4.9 Hz, 1H), 5.91 (s, 1H), 5.41-5.13 (m, 2H), 4.31-4.08 (m, 2H), 3.76 (d, J = 5.7 Hz, 2H), 3.41 (d, J = 1.0 Hz, 2H), 2.47 (s, 2H) |
| 149 | 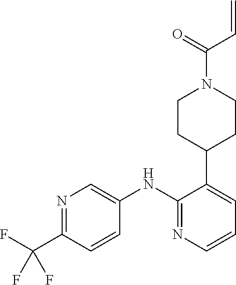 [SMILES: C=CC(=O)N1CCC(c2cccnc2Nc2ccc(C(F)(F)F)nc2)CC1] | — | 2.71 | 0.164 | — | ¹H NMR (400 MHz, DMSO) δ 8.91 (d, J = 2.5 Hz, 1H), 8.72 (s, 1H), 8.26 (dd, J = 8.9, 2.5 Hz, 1H), 8.11 (dd, J = 4.8, 1.7 Hz, 1H), 7.77 (d, J = 8.7 Hz, 1H), 7.65 (dd, J = 7.7, 1.8 Hz, 1H), 6.98 (dd, J = 7.6, 4.8 Hz, 1H), 6.87 (dd, J = 16.7, 10.4 Hz, 1H), 6.13 (dd, J = 16.7, 2.5 Hz, 1H), 5.70 (dd, J = 10.4, 2.5 Hz, 1H), 4.64 (d, J = 13.0 Hz, 1H), 4.23 (d, J = 13.6 Hz, 1H), 3.25 (q, J = 12.7 Hz, 2H), 2.79 (t, |
| | | | | | | J = 12.7 Hz, 1H), 1.87 |
| | | | | | | (d, J = 12.9 Hz, 2H), |
| | | | | | | 1.64-1.40 (m, 2H). |
| 150 | 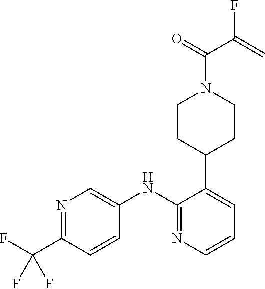 [SMILES: C=C(F)C(=O)N1CCC(c2cccnc2Nc2ccc(C(F)(F)F)nc2)CC1] | | 39.5 | 4.43 | | ¹H NMR (400 MHz, DMSO) δ 8.90 (d, J = 2.5 Hz, 1H), 8.73 (s, 1H), 8.25 (dd, J = 8.5, 2.5 Hz, 1H), 8.12 (dd, J = 4.8, 1.8 Hz, 1H), 7.77 (d, J = 8.7 Hz, 1H), 7.68 (dd, J = 7.7, 1.8 Hz, 1H), 6.99 (dd, J = 7.6, 4.8 Hz, 1H), 5.35- 5.12 (m, 2H), 4.48 (s, 1H), 4.07 (s, 1H), 3.28 (d, J = 12.0 Hz, 2H), 3.01-2.82 (m, 1H), 1.90 (d, J = 13.0 Hz, 2H), 1.58 (s, 2H). |
| 151 | 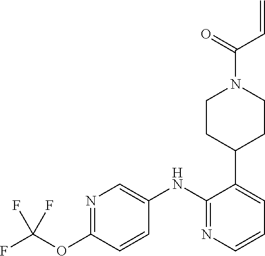 [SMILES: C=CC(=O)N1CCC(c2cccnc2Nc2ccc(OC(F)(F)F)nc2)CC1] | | 1.05 | 0.188 | | ¹H NMR (400 MHz, DMSO) δ 8.59-8.35 (m, 2H), 8.19 (s, 1H), 8.02 (d, J = 9.5 Hz, 1H), 7.57 (d, J = 6.9 Hz, 1H), 7.24 (s, 1H), 6.96-6.78 (m, 2H), 6.12 (s, 1H), 5.69 (s, 1H), 4.63 (s, 1H), 4.22 (s, 1H), 3.21 (s, 2H), 2.79 (d, J = 12.3 Hz, 1H), 1.87 (s, 2H), 1.49 (s, 2H). |
| 152 | 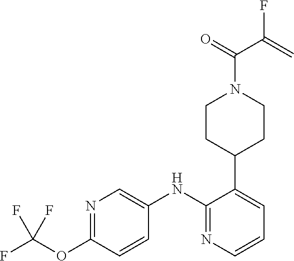 [SMILES: C=C(F)C(=O)N1CCC(c2cccnc2Nc2ccc(OC(F)(F)F)nc2)CC1] | — | 22.25 | 13.46 | — | ¹H NMR (400 MHz, DMSO) δ 8.54 (d, J = 2.8 Hz, 1H), 8.43 (s, 1H), 8.19 (dd, J = 8.9, 2.8 Hz, 1H), 8.03 (dd, J = 4.8, 1.7 Hz, 1H), 7.60 (dd, J = 7.5, 1.8 Hz, 1H), 7.25 (d, J = 8.8 Hz, 1H), 6.89 (dd, J = 7.6, 4.8 Hz, 1H), 5.32- 5.11 (m, 2H), 4.47 (s, 1H), 4.06 (s, 1H), 3.22 (d, J = 11.9 Hz, 1H), 2.91 (s, 1H), 2.72- 2.57 (m, 1H), 1.91 (d, |
| | | | | | | J = 12.9 Hz, 2H), |
| | | | | | | 1.57 (s, 2H). |
| 153 | 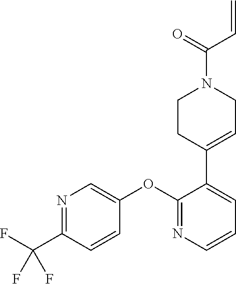 [SMILES: C=CC(=O)N1CC=C(c2cccnc2Oc2ccc(C(F)(F)F)nc2)CC1] | 0.065 | 0.034 | 0.0014 | — | ¹H NMR (400 MHz, DMSO) δ 8.67 (d, J = 2.6 Hz, 1H), 8.08 (dd, J = 4.8, 1.8 Hz, 1H), 7.99 (d, J = 8.6 Hz, 1H), 7.89 (dd, J = 15.5, 8.0 Hz, 2H), 7.27 (dd, J = 7.5, 4.8 Hz, 1H), 6.95- 6.76 (m, 1H), 6.21- 6.07 (m, 2H), 5.71 (d, J = 8.1 Hz, 1H), 4.35- 4.15 (m, 2H), 3.75 (dt, J = 11.5, 5.9 Hz, 2H), 2.62 (s, 2H). |
| 154 | 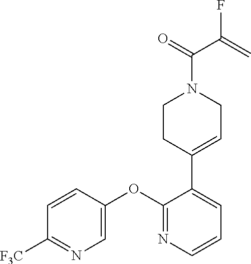 [SMILES: C=C(F)C(=O)N1CC=C(c2cccnc2Oc2ccc(C(F)(F)F)nc2)CC1] | 0.02 | 0.05 | 0.0029 | — | ¹H NMR (400 MHz, DMSO) δ 8.67 (d, J = 2.6 Hz, 1H), 8.09 (dd, J = 4.8, 1.8 Hz, 1H), 8.00 (d, J = 8.6 Hz, 1H), 7.89 (ddd, J = 14.4, 8.1, 2.3 Hz, 2H), 7.27 (dd, J = 7.5, 4.8 Hz, 1H), 6.18 (s, 1H), 5.33 (d, J = 16.9 Hz, 2H), 4.34- 4.13 (m, 2H), 3.73 (t, J = 5.6 Hz, 2H), 2.65 (s, 2H). |
| 155 | 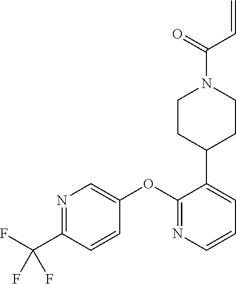 [SMILES: C=CC(=O)N1CCC(c2cccnc2Oc2ccc(C(F)(F)F)nc2)CC1] | 0.018 | 0.015 | 0.0018 | — | ¹H NMR (400 MHz, DMSO) δ 8.67 (d, J = 2.6 Hz, 1H), 8.01 (dd, J = 6.5, 4.2 Hz, 2H), 7.93-7.79 (m, 2H), 7.21 (dd, J = 7.5, 4.8 Hz, 1H), 6.86 (dd, J = 16.7, 10.5 Hz, 1H), 6.12 (dd, J = 16.7, 2.5 Hz, 1H), 5.69 (dd, J = 10.4, 2.4 Hz, 1H), 4.63 (d, J = 13.1 Hz, 1H), 4.22 (d, J = 13.7 Hz, 1H), 3.24 (d, J = 15.0 Hz, 2H), 2.77 (t, J = 12.8 Hz, 1H), 1.95 (d, J = 12.9 Hz, 2H), 1.63 (t, J = 14.4 |
| | | | | | | Hz, 2H). |
| 156 | 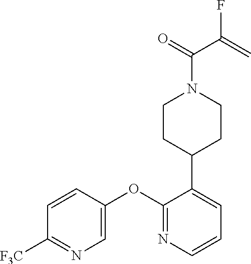 [SMILES: C=C(F)C(=O)N1CCC(c2cccnc2Oc2ccc(C(F)(F)F)nc2)CC1] | — | 0.083 | 0.0137 | — | ¹H NMR (400 MHz, DMSO) δ 8.67 (d, J = 2.6 Hz, 1H), 8.08- 7.97 (m, 2H), 7.89 (ddd, J = 9.6, 8.0, 2.3 Hz, 2H), 7.22 (dd, J = 7.5, 4.9 Hz, 1H), 5.33- 5.10 (m, 2H), 4.46 (s, 1H), 4.05 (s, 1H), 3.32- 3.22 (m, 2H), 2.91 (s, 1H), 1.99 (d, J = 13.0 Hz, 2H), 1.69 (s, 2H). |
| 157 | 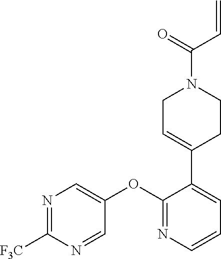 [SMILES: C=CC(=O)N1CC=C(c2cccnc2Oc2cnc(C(F)(F)F)nc2)CC1] | 0.013 | 0.132 | 0.0016 | — | ¹H NMR (400 MHz, DMSO) δ 9.09 (s, 2H), 8.09 (dd, J = 4.9, 1.8 Hz, 1H), 7.90 (d, J = 7.4 Hz, 1H), 7.30 (dd, J = 7.5, 4.8 Hz, 1H), 6.95- 6.74 (m, 1H), 6.26- 6.11 (m, 2H), 5.72 (d, J = 10.2 Hz, 1H), 4.39- 4.14 (m, 2H), 3.77 (dt, J = 11.1, 5.7 Hz, 2H), 2.66 (d, J = 15.4 Hz, 2H). |
| 158 | 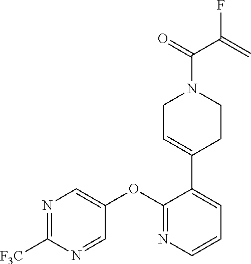 [SMILES: C=C(F)C(=O)N1CC=C(c2cccnc2Oc2cnc(C(F)(F)F)nc2)CC1] | — | 0.027 | 0.0039 | — | ¹H NMR (400 MHz, DMSO) δ 9.09 (s, 2H), 8.10 (dd, J = 4.9, 1.8 Hz, 1H), 7.91 (dd, J = 7.5, 1.9 Hz, 1H), 7.31 (dd, J = 7.5, 4.9 Hz, 1H), 6.22 (s, 1H), 5.42- 5.14 (m, 2H), 4.37- 4.11 (m, 2H), 3.75 (t, J = 5.7 Hz, 2H), 2.68 (s, 2H). |
| 159 | 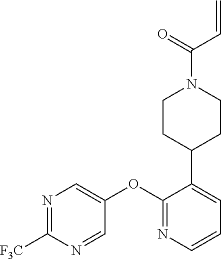 [SMILES: C=CC(=O)N1CCC(c2cccnc2Oc2cnc(C(F)(F)F)nc2)CC1] | 0.051 | 0.022 | 0.0018 | — | ¹H NMR (400 MHz, DMSO) δ 9.09 (s, 2H), 8.03 (dd, J = 4.8, 1.8 Hz, 1H), 7.89 (dd, J = 7.5, 1.8 Hz, 1H), 7.25 (dd, J = 7.5, 4.8 Hz, 1H), 6.87 (dd, J = 16.7, 10.4 Hz, 1H), 6.13 (dd, J = 16.7, 2.5 Hz, 1H), 5.70 (dd, J = 10.5, 2.5 Hz, 1H), 4.64 (d, J = 13.0 Hz, 1H), 4.23 (d, J = 13.7 Hz, 1H), 3.33-3.15 (m, 2H), 2.77 (t, J = 12.7 Hz, 1H), 1.97 (d, |
| | | | | | | J = 12.7 Hz, 2H), 1.71- |
| | | | | | | 1.48 (m, 2H). |
| 160 | 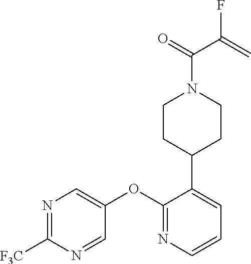 [SMILES: C=C(F)C(=O)N1CCC(c2cccnc2Oc2cnc(C(F)(F)F)nc2)CC1] | — | 2.94 | 1.65 | — | ¹H NMR (400 MHz, DMSO) δ 9.09 (s, 2H), 8.03 (dd, J = 4.8, 1.8 Hz, 1H), 7.92 (d, J = 7.4 Hz, 1H), 7.26 (dd, J = 7.5, 4.8 Hz, 1H), 5.34- 5.11 (m, 2H), 4.47 (s, 1H), 4.06 (s, 1H), 3.30 (d, J = 14.6 Hz, 2H), 2.90 (s, 1H), 2.01 (d, J = 13.0 Hz., 2H), 1.70 (s, 2H). |
| 161 | 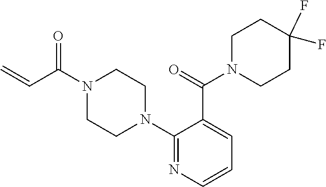 [SMILES: C=CC(=O)N1CCN(c2ncccc2C(=O)N2CCC(F)(F)CC2)CC1] | — | >20 | 1.73 | — | ¹H NMR (400 MHz, DMSO) δ 8.28 (dd, J = 4.8, 1.9 Hz, 1H), 7.62 (dd, J = 7.4, 1.9 Hz, 1H), 6.95 (dd, J = 7.4, 4.8 Hz, 1H), 6.83 (dd, J = 16.7, 10.5 Hz, 1H), 6.15 (dd, J = 16.7, 2.4 Hz, 1H), 5.71 (dd, J = 10.4, 2.4 Hz, 1H), 4.00- 3.85 (m, 1H), 3.82- 3.50 (m, 5H), 3.34- |
| | | | | | | 3.21 (m, 6H), 2.15- |
| | | | | | | 1.87 (m, 4H). |
| 162 | 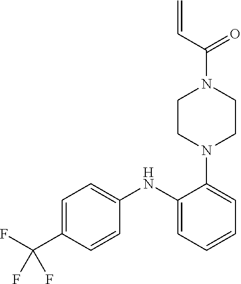 [SMILES: C=CC(=O)N1CCN(c2ccccc2Nc2ccc(C(F)(F)F)cc2)CC1] | — | 0.192 | 0.0011 | — | ¹H NMR (400 MHz, DMSO) δ 7.94 (s, 1H), 7.50 (d, J = 8.5 Hz, 2H), 7.33-7.21 (m, 1H), 7.16-6.95 (m, 5H), 6.82 (dd, J = 16.6, 10.4 Hz, 1H), 6.13 (dd, J = 16.7, 2.4 Hz, 1H), 5.69 (dd, J = 10.4, 2.4 Hz, 1H), 3.60 (s, 4H), 2.92-2.76 (m, 4H). |
| 163 | 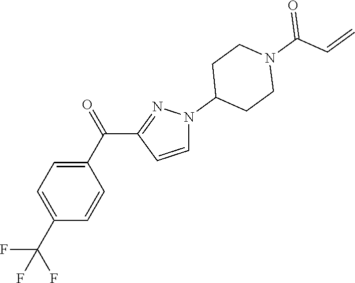 [SMILES: C=CC(=O)N1CCC(n2ccc(C(=O)c3ccc(C(F)(F)F)cc3)n2)CC1] | — | 5 | 10 | — | ¹H NMR (400 MHz, DMSO) δ 8.31 (s, 2H), 8.09 (s, 1H), 7.94 (d, J = 7.9 Hz, 2H), 7.01- 6.80 (m, 2H), 5.70 (s, 1H), 4.72-4.49 (m, 2H), 4.19 (s, 1H), 2.86 (s, 1H), 2.14 (s, 2H), 1.88 (s, 2H). |
| 164 | 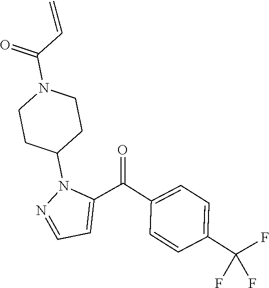 [SMILES: C=CC(=O)N1CCC(n2nccc2C(=O)c2ccc(C(F)(F)F)cc2)CC1] | 0.045 | — | 0.0004 | — | ¹H NMR (400 MHz, DMSO) δ 8.06 (d, J = 8.1 Hz, 2H), 7.97 (d, J = 8.2 Hz, 2H), 7.69 (d, J = 2.0 Hz, 1H), 6.88 (dd, J = 16.7, 10.5 Hz, 1H), 6.80 (d, J = 2.0 Hz, 1H), 6.15 (dd, J = 16.7, 2.4 Hz, 1H), 5.71 (dd, J = 10.4, 2.4 Hz, 1H), 5.27-5.10 (m, 1H), 4.58 (d, J = 13.3 Hz, 1H), 4.23 (d, J = 13.8 Hz, 1H), 3.24 (d, J = 13.1 Hz, 1H), 2.84 (t, J = 12.7 Hz, 1H), 2.14-1.83 (m, 4H). |
| 165 | 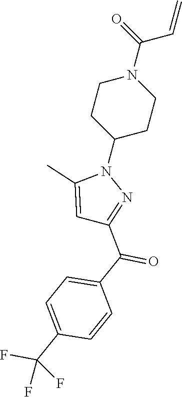 [SMILES: C=CC(=O)N1CCC(n2nc(C(=O)c3ccc(C(F)(F)F)cc3)cc2C)CC1] | — | 5 | 0.0198 | — | ¹H NMR (400 MHz, DMSO) δ 8.30 (d, J = 8.0 Hz, 2H), 7.92 (d, J = 8.0 Hz, 2H), 6.91- 6.80 (m, 1H), 6.75 (d, J = 1.3 Hz, 1H), 6.13 (ddd, J = 16.8, 2.5, 1.1 Hz, 1H), 5.70 (ddd, J = 10.5, 2.5, 1.0 Hz, 1H), 4.60 (q, J = 14.4 Hz, 2H), 4.20 (d, J = 13.8 Hz, 1H), 3.26 (d, J = 11.9 Hz, 1H), 2.87 (t, J = 12.7 Hz, 1H), 2.02 (d, J = 12.6 Hz, 2H), 1.87 (d, J = 13.9 Hz, 2H). |
| 166 | 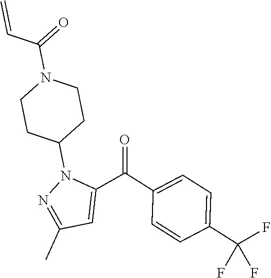 [SMILES: C=CC(=O)N1CCC(n2nc(C)cc2C(=O)c2ccc(C(F)(F)F)cc2)CC1] | 0.015 | 0.020 | 0.0006 | — | ¹H NMR (400 MHz, DMSO) δ 8.04 (d, J = 8.1 Hz, 2H), 7.96 (d, J = 8.2 Hz, 2H), 6.88 (dd, J = 16.7, 10.4 Hz, 1H), 6.57 (s, 1H), 6.14 (dd, J = 16.7, 2.5 Hz, 1H), 5.71 (dd, J = 10.4, 2.4 Hz, 1H), 5.12 (td, J = 11.2, 5.5 Hz, 1H), 4.57 (d, J = 13.2 Hz, 1H), 4.21 (d, J = 13.9 Hz, 1H), 3.23 (t, J = 12.8 Hz, 1H), 2.81 (t, J = 12.9 Hz, 1H), 2.23 (s, 3H), 2.08-1.73 (m, 4H). |
| 167 | 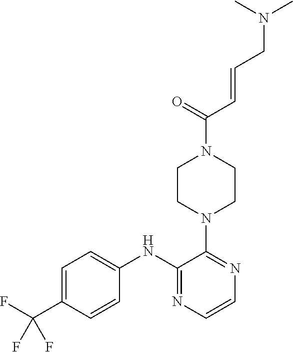 [SMILES: CN(C)C/C=C/C(=O)N1CCN(c2nccnc2Nc2ccc(C(F)(F)F)cc2)CC1] | — | 0.108 | 1 | 10 | ¹H NMR (400 MHz, DMSO) δ 8.63 (d, J = 2.4 Hz, 1H), 8.02- 7.89 (m, 3H), 7.85 (d, J = 2.8 Hz, 1H), 7.65 (d, J = 8.6 Hz, 2H), 6.82 (d, J = 15.2 Hz, 1H), 6.65 (dt, J = 15.0, 6.6 Hz, 1H), 3.79 (s, 4H), 3.52 (s, 6H), 3.15 (d, J = 6.1 Hz, 6H). |
| 168 | 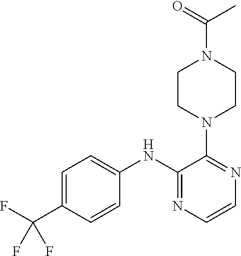 [SMILES: CC(=O)N1CCN(c2nccnc2Nc2ccc(C(F)(F)F)cc2)CC1] | — | >10 | 0.487 | >20 | ¹H NMR (400 MHz, DMSO) δ 8.61 (s, 1H), 7.94-7.87 (m, 3H), 7.85 (d, J = 2.8 Hz, 1H), 7.65 (d, J = 8.6 Hz, 2H), 3.68 (q, J = 4.9 Hz, 4H), 3.21- 3.03 (m, 4H), 2.05 (s, 3H). |
| 169 | 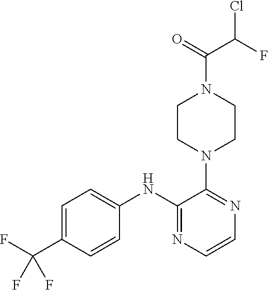 [SMILES: O=C(C(F)Cl)N1CCN(c2nccnc2Nc2ccc(C(F)(F)F)cc2)CC1] | — | 0.037 | 0.0131 | >10 | ¹H NMR (400 MHz, DMSO) δ 8.63 (s, 1H), 7.98-7.87 (m, 3H), 7.85 (d, J = 2.8 Hz, 1H), 7.65 (d, J = 8.6 Hz, 2H), 7.44-7.27 (m, 1H), 3.87-3.61 (m, 4H), 3.18 (dt, J = 15.4, 4.3 Hz, 4H). |
| 170 | 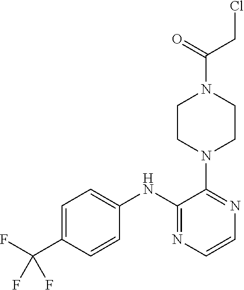 [SMILES: O=C(CCl)N1CCN(c2nccnc2Nc2ccc(C(F)(F)F)cc2)CC1] | — | 0.173 | 0.267 | 0.76 | ¹H NMR (400 MHz, DMSO) δ 8.63 (s, 1H), 7.97-7.86 (m, 3H), 7.85 (d, J = 2.8 Hz, 1H), 7.65 (d, J = 8.6 Hz, 2H), 4.45 (s, 2H), 3.82-3.65 (m, 4H), 3.26-3.05 (m, 4H). |
| 171 | 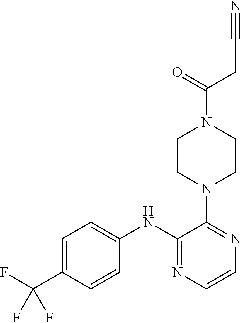 [SMILES: N#CCC(=O)N1CCN(c2nccnc2Nc2ccc(C(F)(F)F)cc2)CC1] | — | 5.25 | 2.22 | >20 | ¹H NMR (400 MHz, DMSO) δ 8.63 (s, 1H), 7.93 (s, 1H), 7.91-7.88 (m, 2H), 7.84 (d, J = 2.8 Hz, 1H), 7.65 (d, J = 8.5 Hz, 2H), 4.12 (s, 2H), 3.71 (t, J = 5.3 Hz, 2H), 3.59 (d, J = 5.6 Hz, 2H), 3.23-3.07 (m, 4H). |
| 172 | 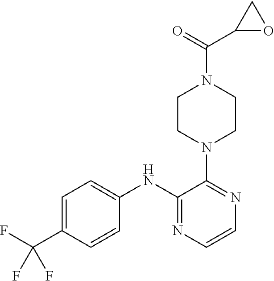 [SMILES: O=C(C1CO1)N1CCN(c2nccnc2Nc2ccc(C(F)(F)F)cc2)CC1] | — | 1.4 | 0.0032 | 10 | ¹H NMR (400 MHz, DMSO) δ 8.64 (s, 1H), 7.98-7.88 (m, 3H), 7.85 (d, J = 2.8 Hz, 1H), 7.65 (d, J = 8.6 Hz, 2H), 3.97-3.80 (m, 3H), 3.71 (d, J = 6.6 Hz, 2H), 3.26- 3.08 (m, 4H), 2.93 (dd, J = 6.3, 4.3 Hz, 1H), 2.81 (dd, J = 6.3, 2.6 Hz, 1H). |
| 173 | 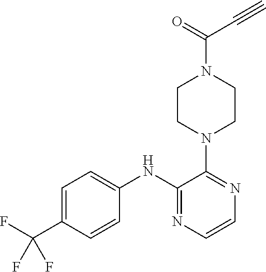 [SMILES: C#CC(=O)N1CCN(c2nccnc2Nc2ccc(C(F)(F)F)cc2)CC1] | — | 0.507 | 0.0365 | 0.202 | ¹H NMR (400 MHz, DMSO) δ 8.61 (s, 1H), 7.96-7.89 (m, 3H), 7.85 (d, J = 2.8 Hz, 1H), 7.65 (d, J = 8.5 Hz, 2H), 4.61 (s, 1H), 4.00-3.88 (m, 2H), 3.75 (dd, J = 6.3, 4.0 Hz, 2H), 3.22 (t, J = 5.2 Hz, 2H), 3.14 (t, J = 5.2 Hz, 2H). |
| 174 | 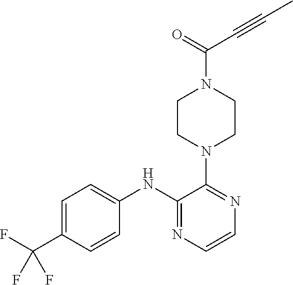 [SMILES: CC#CC(=O)N1CCN(c2nccnc2Nc2ccc(C(F)(F)F)cc2)CC1] | — | >20 | >20 | >10 | ¹H NMR (400 MHz, DMSO) δ 8.61 (s, 1H), 7.99-7.88 (m, 3H), 7.85 (d, J = 2.8 Hz, 1H), 7.65 (d, J = 8.6 Hz, 2H), 3.92 (t, J = 5.1 Hz, 2H), 3.73 (t, J = 5.1 Hz, 2H), 3.27-3.08 (m, 4H), 2.04 (s, 3H). |
| 175 | 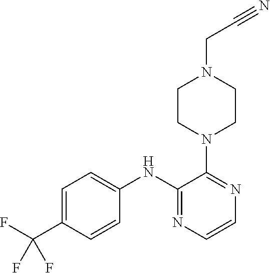 [SMILES: N#CCN1CCN(c2nccnc2Nc2ccc(C(F)(F)F)cc2)CC1] | — | >10 | 0.833 | >20 | ¹H NMR (400 MHz, DMSO) δ 8.59 (s, 1H), 7.93-7.78 (m, 4H), 7.63 (d, J = 8.5 Hz, 2H), 3.81 (s, 2H), 3.21 (s, 4H), 2.73 (t, J = 4.8 Hz, 4H). |
| 176 | 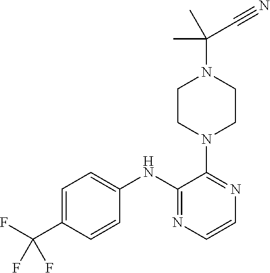 [SMILES: CC(C)(C#N)N1CCN(c2nccnc2Nc2ccc(C(F)(F)F)cc2)CC1] | — | 9.95 | 2.5 | >10 | ¹H NMR (400 MHz, DMSO) δ 8.63 (s, 1H), 7.97-7.80 (m, 4H), 7.62 (d, J = 8.6 Hz, 2H), 3.27-3.09 (m, 4H), 2.81 (t, J = 4.7 Hz, 4H), 1.49 (s, 6H). |
| 177 | 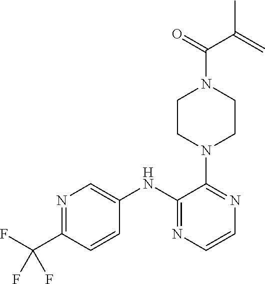 [SMILES: C=C(C)C(=O)N1CCN(c2nccnc2Nc2ccc(C(F)(F)F)nc2)CC1] | — | >20 | >20 | — | ¹H NMR (400 MHz, DMSO) δ 9.03 (d, J = 2.5 Hz, 1H), 8.86 (s, 1H), 8.39 (dd, J = 8.7, 2.5 Hz, 1H), 7.90 (q, J = 2.8 Hz, 2H), 7.83 (d, J = 8.7 Hz, 1H), 5.25- 5.19 (m, 1H), 5.06- 4.99 (m, 1H), 3.72 (s, 4H), 3.17 (t, J = 5.1 Hz, 4H), 1.89 (d, J = 1.3 Hz, 3H). |
| 178 | 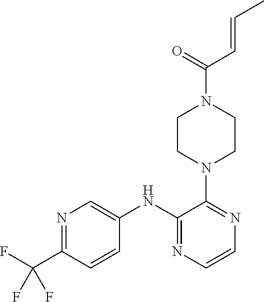 [SMILES: C/C=C/C(=O)N1CCN(c2nccnc2Nc2ccc(C(F)(F)F)nc2)CC1] | — | 0.884 | 0.0027 | — | ¹H NMR (400 MHz, DMSO) δ 9.03 (d, J = 2.5 Hz, 1H), 8.87 (s, 1H), 8.39 (dd, J = 8.6, 2.5 Hz, 1H), 7.90 (s, 2H), 7.83 (d, J = 8.7 Hz, 1H), 6.80-6.65 (m, 1H), 6.56 (dd, J = 15.0, 1.7 Hz, 1H), 3.77 (s, 4H), 3.17 (s, 4H), 1.86 (dd, J = 6.8, 1.5 Hz, 3H). |
| 179 | 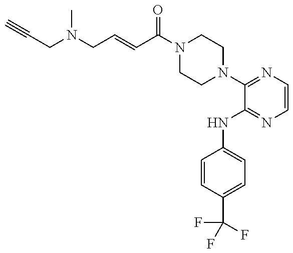 [SMILES: C#CCN(C)C/C=C/C(=O)N1CCN(c2nccnc2Nc2ccc(C(F)(F)F)cc2)CC1] | — | 2.48 | >10 | >20 | ¹H NMR (400 MHz, DMSO) δ 8.62 (s, 1H), 7.96-7.82 (m, 4H), 7.65 (d, J = 8.6 Hz, 2H), 6.71-6.54 (m, 2H), 3.78 (dd, J = 10.5, 5.9 Hz, 4H), 3.31 (s, 2H), 3.20-3.13 (m, 6H), 2.21 (s, 3H), 1.05 (d, J = 6.1 Hz, 1H). |
| 180 | 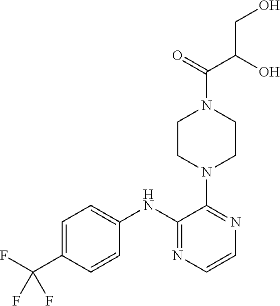 [SMILES: O=C(C(O)CO)N1CCN(c2nccnc2Nc2ccc(C(F)(F)F)cc2)CC1] | — | >20 | >20 | — | ¹H NMR (400 MHz, DMSO) δ 8.62 (s, 1H), 7.98-7.79 (m, 4H), 7.65 (d, J = 8.5 Hz, 2H), 4.99 (d, J = 7.3 Hz, 1H), 4.75 (t, J = 5.8 Hz, 1H), 4.39 (dt, J = 7.0, 5.6 Hz, 1H), 3.87- 3.66 (m, 4H), 3.60- 3.44 (m, 2H), 3.14 (d, J = 14.9 Hz, 4H). |
| 181 | 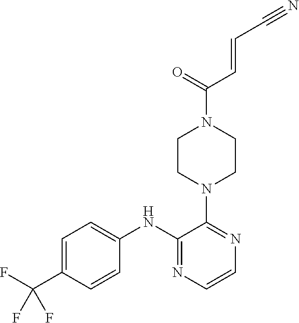 [SMILES: N#C/C=C/C(=O)N1CCN(c2nccnc2Nc2ccc(C(F)(F)F)cc2)CC1] | 0.026 | 0.034 | 0.00044 | — | ¹H NMR (400 MHz, DMSO) δ 8.64 (s, 1H), 7.96-7.78 (m, 5H), 7.65 (d, J = 8.6 Hz, 2H), 6.59 (d, J = 15.9 Hz, 1H), 3.92-3.69 (m, 4H), 3.18 (d, J = 5.1 Hz, 4H). |
| 182 | 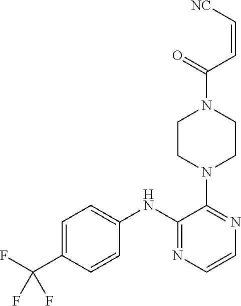 [SMILES: N#C/C=C\C(=O)N1CCN(c2nccnc2Nc2ccc(C(F)(F)F)cc2)CC1] | — | 0.034 | 0.0012 | — | ¹H NMR (400 MHz, DMSO) δ 8.64 (s, 1H), 7.96-7.85 (m, 3H), 7.85 (d, J = 2.8 Hz, 1H), 7.65 (d, J = 8.6 Hz, 2H), 7.45 (d, J = 11.6 Hz, 1H), 6.22 (d, J = 11.6 Hz, 1H), 3.87- 3.67 (m, 4H), 3.18 (q, J = 5.9 Hz, 4H). |
| 183 | 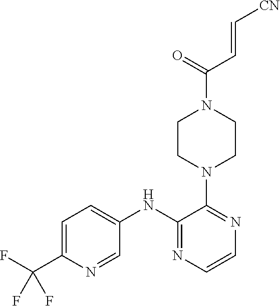 [SMILES: N#C/C=C/C(=O)N1CCN(c2nccnc2Nc2ccc(C(F)(F)F)nc2)CC1] | — | 0.422 | 0.0339 | — | ¹H NMR (400 MHz, DMSO) δ 9.03 (d, J = 2.5 Hz, 1H), 8.89 (s, 1H), 8.39 (d, J = 8.4 Hz, 1H), 7.94-7.76 (m, 3H), 6.59 (d, J = 15.8 Hz, 1H), 3.92- 3.69 (m, 4H), 3.21 (d, J = 5.2 Hz, 4H). |
| 184 | 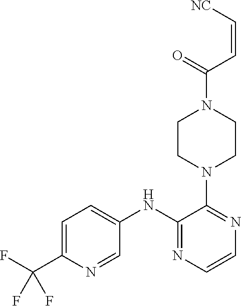 [SMILES: N#C/C=C\C(=O)N1CCN(c2nccnc2Nc2ccc(C(F)(F)F)nc2)CC1] | — | 1.45 | 0.164 | — | ¹H NMR (400 MHz, DMSO) δ 9.04 (d, J = 2.5 Hz, 1H), 8.89 (s, 1H), 8.40 (dd, J = 8.7, 2.5 Hz, 1H), 7.91 (q, J = 2.8 Hz, 2H), 7.84 (d, J = 8.7 Hz, 1H), 7.45 (d, J = 11.7 Hz, 1H), 6.23 (d, J = 11.6 Hz, 1H), 3.84-3.62 (m, 4H), 3.22 (q, J = 5.5 Hz, 4H). |
| 185 | 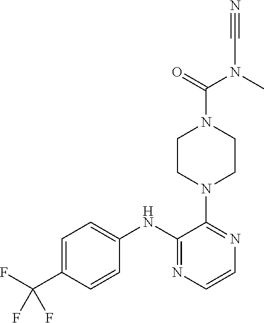 [SMILES: CN(C#N)C(=O)N1CCN(c2nccnc2Nc2ccc(C(F)(F)F)cc2)CC1] | — | >20 | >20 | — | ¹H NMR (400 MHz, DMSO) δ 8.62 (s, 1H), 7.99-7.87 (m, 3H), 7.85 (d, J = 2.7 Hz, 1H), 7.65 (d, J = 8.6 Hz, 2H), 3.70 (dd, J = 6.6, 3.6 Hz, 4H), 3.19 (dd, J = 6.4, 3.7 Hz, 4H), 3.12 (s, 3H). |
| 186 | 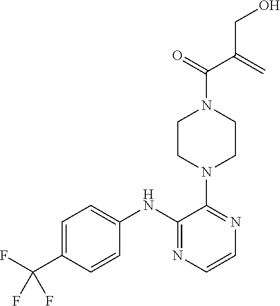 [SMILES: C=C(CO)C(=O)N1CCN(c2nccnc2Nc2ccc(C(F)(F)F)cc2)CC1] | — | >20 | >20 | — | ¹H NMR (400 MHz, DMSO) δ 8.61 (s, 1H), 7.96-7.87 (m, 3H), 7.84 (d, J = 2.8 Hz, 1H), 7.65 (d, J = 8.6 Hz, 2H), 5.37 (q, J = 1.6 Hz, 1H), 5.14- 5.08 (m, 2H), 4.09 (dt, J = 5.8, 1.5 Hz, 2H), 3.75 (t, J = 4.9 Hz, 4H), 3.14 (t, J = 5.1 Hz, 4H). |
| 187 | 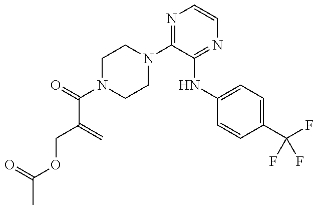 [SMILES: C=C(COC(C)=O)C(=O)N1CCN(c2nccnc2Nc2ccc(C(F)(F)F)cc2)CC1] | — | 7.41 | 36.62 | — | ¹H NMR (400 MHz, DMSO) δ 8.62 (s, 1H), 7.96-7.86 (m, 3H), 7.85 (d, J = 2.8 Hz, 1H), 7.65 (d, J = 8.6 Hz, 2H), 5.54 (s, 1H), 5.33 (s, 1H), 4.71 (s, 2H), 3.76 (s, 4H), 3.15 (t, J = 5.0 Hz, 4H). |
| 188 | 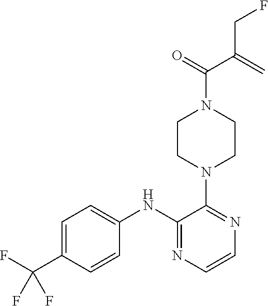 [SMILES: C=C(CF)C(=O)N1CCN(c2nccnc2Nc2ccc(C(F)(F)F)cc2)CC1] | — | 0.013 | 0.0023 | — | ¹H NMR (400 MHz, DMSO) δ 8.62 (s, 1H), 7.98-7.87 (m, 3H), 7.84 (d, J = 2.7 Hz, 1H), 7.65 (d, J = 8.6 Hz, 2H), 5.65 (d, J = 3.9 Hz, 1H), 5.43 (s, 1H), 5.14 (s, 1H), 5.03 (s, 1H), 3.78 (s, 4H), 3.15 (t, J = 5.0 Hz, 4H). |
| 189 | 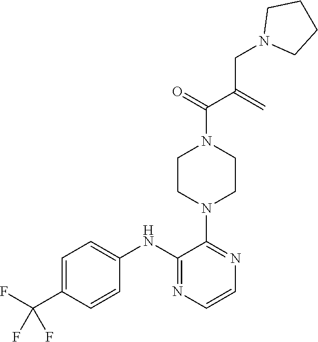 [SMILES: C=C(CN1CCCC1)C(=O)N1CCN(c2nccnc2Nc2ccc(C(F)(F)F)cc2)CC1] | — | 5.36 | 2.12 | 9.3 | ¹H NMR (400 MHz, DMSO) δ 9.53 (s, 1H), 8.64 (d, J = 13.8 Hz, 1H), 7.97-7.80 (m, 4H), 7.65 (t, J = 7.7 Hz, 2H), 5.99-5.65 (m, 1H), 4.06-3.64 (m, 4H), 3.24-3.13 (m, 4H), 2.91-2.57 (m, 4H), 2.08 (s, 5H), 1.34- 1.19 (m, 1H). |
| 190 | 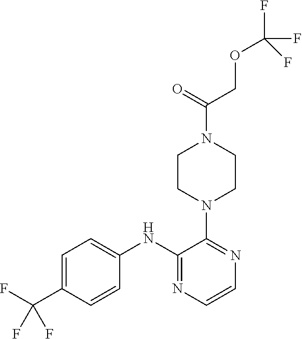 [SMILES: O=C(COC(F)(F)F)N1CCN(c2nccnc2Nc2ccc(C(F)(F)F)cc2)CC1] | — | 0.994 | 0.0127 | — | ¹H NMR (400 MHz, DMSO) δ 8.63 (s, 1H), 7.96-7.80 (m, 4H), 7.65 (d, J = 8.6 Hz, 2H), 5.00 (s, 2H), 3.71 (s, 2H), 3.58 (s, 2H), 3.23-3.06 (m, 4H). |
| 191 | 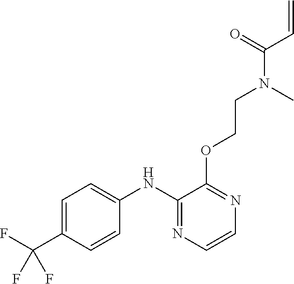 [SMILES: C=CC(=O)N(C)CCOc1nccnc1Nc1ccc(C(F)(F)F)cc1] | — | 0.047 | 0.0043 | 10 | ¹H NMR (400 MHz, DMSO) δ 8.82 (d, J = 8.6 Hz, 1H), 8.18- 8.01 (m, 2H), 7.79 (t, J = 3.4 Hz, 1H), 7.73- 7.52 (m, 3H), 6.96- 6.70 (m, 1H), 6.21- 5.99 (m, 1H), 5.74- 5.52 (m, 1H), 4.62- 4.48 (m, 2H), 3.96- 3.76 (m, 2H), 3.20- 2.91 (m, 3H). |
| 192 | 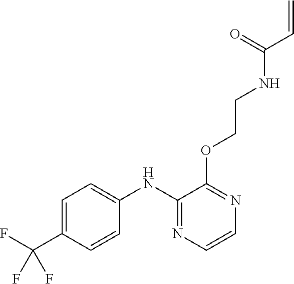 [SMILES: C=CC(=O)NCCOc1nccnc1Nc1ccc(C(F)(F)F)cc1] | — | 1.78 | 0.0179 | >20 | ¹H NMR (400 MHz, DMSO) δ 8.86 (s, 1H), 8.45 (d, J = 6.2 Hz, 1H), 8.12 (d, J = 8.5 Hz, 2H), 7.79 (d, J = 3.0 Hz, 1H), 7.69 (d, J = 8.6 Hz, 2H), 7.60 (d, J = 3.0 Hz, 1H), 6.27 (dd, J = 17.1, 10.0 Hz, 1H), 6.15 (dd, J = 17.1, 2.3 Hz, 1H), 5.64 (dd, J = 10.0, 2.3 Hz, 1H), 4.42 (t, J = 5.5 Hz, 2H), 3.65 (q, J = 5.6 Hz, 2H). |
| 193 | 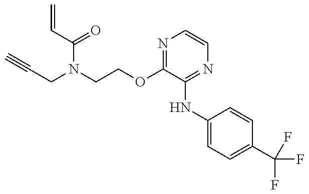 [SMILES: C#CCN(CCOc1nccnc1Nc1ccc(C(F)(F)F)cc1)C(=O)C=C] | — | >20 | 0.203 | — | ¹H NMR (400 MHz, DMSO) δ 9.72-9.50 (m, 1H), 8.22 (d, J = 8.5 Hz, 2H), 7.65 (d, J = 8.6 Hz, 2H), 7.17- 6.86 (m, 2H), 6.82- 6.56 (m, 1H), 6.19- 5.99 (m, 1H), 5.78- 5.55 (m, 1H), 4.32 (dd, J = 17.4, 2.5 Hz, 2H), 4.13 (q, J = 5.3 Hz, 2H), 3.92-3.70 (m, 2H). |
| 194 | 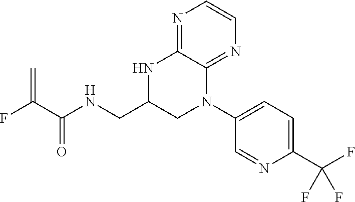 [SMILES: C=C(F)C(=O)NCC1CN(c2ccc(C(F)(F)F)nc2)c2nccnc2N1] | — | >20 | 0.238 | — | ¹H NMR (400 MHz, DMSO) δ 9.19 (d, J = 7.9 Hz, 1H), 8.68 (s, 2H), 7.79 (d, J = 10.1 Hz, 1H), 6.99 (d, J = 7.6 Hz, 1H), 6.52 (d, J = 9.3 Hz, 1H), 5.68- 5.43 (m, 1H), 5.28 (s, 1H), 4.38 (s, 1H), 4.20- 4.06 (m, 1H), 3.91 (s, 1H), 3.23 (s, 2H). |

### PHARMACEUTICAL COMPOSITIONS

The disclosure also provides a pharmaceutical composition comprising a therapeutically effective amount of one or more compounds as described herein, or a pharmaceutically acceptable salt, stereoisomer, and/or tautomer thereof in admixture with a pharmaceutically acceptable carrier. In some embodiments, the composition further contains, in accordance with accepted practices of pharmaceutical compounding, one or more additional therapeutic agents, pharmaceutically acceptable excipients, diluents, adjuvants, stabilizers, emulsifiers, preservatives, colorants, buffers, flavor imparting agents.

In one embodiment, the pharmaceutical composition comprises a compound selected from those illustrated in Table 1, or a pharmaceutically acceptable salt, stereoisomer, and/or tautomer thereof, and a pharmaceutically acceptable carrier.

The pharmaceutical composition of the present disclosure is formulated, dosed, and administered in a fashion consistent with good medical practice. Factors for consideration in this context include the particular disorder being treated, the particular subject being treated, the clinical condition of the subject, the cause of the disorder, the site of delivery of the agent, the method of administration, the scheduling of administration, and other factors known to medical practitioners.

The “therapeutically effective amount” of a compound or a pharmaceutically acceptable salt, stereoisomer, and/or tautomer thereof that is administered is governed by such considerations, and it is the minimum amount necessary to inhibit YAP transcriptional activity, inhibit one or more of TEA domain family proteins (TEADs), exert oncological and/or immuno-oncological therapeutic activity, and combinations thereof. Such amount may be below the amount that is toxic to normal cells, or the subject as a whole. Generally, the initial therapeutically effective amount of a compound (or a pharmaceutically acceptable salt, stereoisomer, or tautomer thereof) of the present disclosure that is administered is in the range of about 0.01 to about 200 mg/kg or about 0.1 to about 20 mg/kg of patient body weight per day, with the typical initial range being about 0.3 to about 15 mg/kg/day. Oral unit dosage forms, such as tablets and capsules, may contain from about 0.1 mg to about 1000 mg of a compound (or a pharmaceutically acceptable salt, stereoisomer, or tautomer thereof) of the present disclosure. In another embodiment, such dosage forms contain from about 50 mg to about 500 mg of a compound (or a pharmaceutically acceptable salt, stereoisomer, or tautomer thereof) of the present disclosure. In yet another embodiment, such dosage forms contain from about 25 mg to about 200 mg of a compound (or a pharmaceutically acceptable salt, stereoisomer, or tautomer thereof) of the present disclosure. In still another embodiment, such dosage forms contain from about 10 mg to about 100 mg of a compound (or a pharmaceutically acceptable salt, stereoisomer, or tautomer thereof) of the present disclosure. In a further embodiment, such dosage forms contain from about 5 mg to about 50 mg of a compound (or a pharmaceutically acceptable salt, stereoisomer, or tautomer thereof) of the present disclosure. In any of the foregoing embodiments the dosage form can be administered once a day or twice per day.

The compositions of the present disclosure can be administered orally, topically, parenterally, by inhalation or spray or rectally in dosage unit formulations. The term parenteral as used herein includes subcutaneous injections, intravenous, intramuscular, intrasternal injection or infusion techniques.

Suitable oral compositions as described herein include without limitation tablets, troches, lozenges, aqueous or oily suspensions, dispersible powders or granules, emulsion, hard or soft capsules, syrups or elixirs.

In another aspect, also encompassed are pharmaceutical compositions suitable for single unit dosages that comprise a compound of the disclosure or its pharmaceutically acceptable stereoisomer, salt, or tautomer and a pharmaceutically acceptable carrier.

The compositions of the present disclosure that are suitable for oral use may be prepared according to any method known to the art for the manufacture of pharmaceutical compositions. For instance, liquid formulations of the compounds of the present disclosure contain one or more agents selected from the group consisting of sweetening agents, flavoring agents, coloring agents and preserving agents in order to provide pharmaceutically palatable preparations of a compound of the present disclosure.

For tablet compositions, a compound of the present disclosure in admixture with non-toxic pharmaceutically acceptable excipients is used for the manufacture of tablets. Examples of such excipients include without limitation inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known coating techniques to delay disintegration and absorption in the gastrointestinal tract and thereby to provide a sustained therapeutic action over a desired time period. For example, a time delay material such as glyceryl monostearate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

For aqueous suspensions, a compound of the present disclosure is admixed with excipients suitable for maintaining a stable suspension. Examples of such excipients include without limitation are sodium carboxymethylcellulose, methylcellulose, hydroxpropylmethylcellulose, sodium alginate, polyvinylpyrrolidone, gum tragacanth and gum acacia.

Oral suspensions can also contain dispersing or wetting agents, such as naturally-occurring phosphatide, for example, lecithin, or condensation products of an alkylene oxide with fatty acids, for example polyoxyethylene stearate, or condensation products of ethylene oxide with long chain aliphatic alcohols, for example, heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending a compound of the present disclosure in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Sweetening agents such as those set forth above, and flavoring agents may be added to provide palatable oral preparations. These compositions may be preserved by the addition of an anti-oxidant such as ascorbic acid.

Dispersible powders and granules suitable for preparation of an aqueous suspension by the addition of water provide a compound of the present disclosure in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Pharmaceutical compositions of the present disclosure may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monoleate, and condensation reaction products of the said partial esters with ethylene oxide, for example polyoxyethylene sorbitan monoleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative, and flavoring and coloring agents.

The pharmaceutical compositions may be in the form of a sterile injectable, an aqueous suspension or an oleaginous suspension. This suspension may be formulated according to the known art using those suitable dispersing or wetting agents and suspending agents which have been mentioned above. The sterile injectable preparation may also be sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds as described herein may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include cocoa butter and polyethylene glycols.

Compositions for parenteral administrations are administered in a sterile medium. Depending on the vehicle used and concentration the concentration of the drug in the formulation, the parenteral formulation can either be a suspension or a solution containing dissolved drug. Adjuvants such as local anesthetics, preservatives and buffering agents can also be added to parenteral compositions.

### METHODS OF USE

In additional embodiments, the present disclosure provides a method of treating a disease in a subject suffering therefrom. The method comprises administering to the subject a compound or pharmaceutically acceptable salt thereof as described herein. In some embodiments, the disease is one caused by aberrant activities caused by TEA domain family proteins TEAD1, TEAD2, TEAD3, and/or TEAD4. In additional embodiments, the disease is associated with constitutive activation of YAP. Specific examples of diseases include Diabetic foot ulcer (DFU), Venous Ulcer (Stasis Ulcer), Pressure Ulcers, Full or partial thickness burns, Eczema, Psoriasis, Cellulitis, Impetigo, Atopic dermatitis, Epidermolysis Bullosa, Lichen Sclerosis, Ichthyosis, Vitiligo, Acral peeling skin syndrome, Blau syndrome, Primary cutaneous amyloidosis, Cutaneous abscess, Blepharitis, Furunculosis, Capillaritis, Cellulitis, Corneal Abrasion, Corneal Erosion, Xerosis, Lichen Planus, Lichen Simplex Chronicus, Idiopathic pulmonary fibrosis (IPF), Acute respiratory distress syndrome (ARDS), Chronic Obstructive Pulmonary Disease (COPD), Emphysema, Silicosis, Asbestosis, Pneumoconiosis, Aluminosis, Bauxite fibrosis, Berylliosis, Siderosis, Stannosis, Pulmonary Talcosis, Labrador lung (mixed dust Pneumoconiosis), Sarcoidosis, Hypersensitivity pneumonitis (HP)/extrinsic allergic alveolitis (EAA), Desquamative interstitial pneumonia (DIP), Respiratory bronchiolitis interstitial lung disease (RBILD), Acute interstitial pneumonia (AIP), Nonspecific interstitial pneumonia (NSIP), Cryptogenic organizing pneumonia (COP=idiopathic BOOP), Secondary organizing pneumonia (BOOP), Lymphoid interstitial pneumonia (LIP), Idiopathic interstitial pneumonia: unspecified, Hypereosinophilic lung diseases, Tuberculosis (TB), Pulmonary Edema, Interstitial Lung Disease, Cryptogenic Organizing Pneumonia (COP), E-cigarette or Vaping Use-Associated Lung Injury (EVALI), Hantavirus Pulmonary Syndrome (HPS), Histoplasmosis, Legionnaires' Disease, MAC Lung Disease, Alpha-1 Antitrypsin Deficiency, Aspergillosis, Lymphangioleiomyomatosis (LAM), Middle Eastern Respiratory Syndrome (MERS), Nontuberculous Mycobacterial Lung Disease (NTM), Pulmonary Embolism Goodpasture syndrome, idiopathic pulmonary hemosiderosis, Alveolar proteinosis, Pulmonary amyloidosis, Primary pulmonary lymphoma, Primary ciliary dyskinesia (without or with situs inversus), Rare cause of hypersensitivity pneumonitis (all causes other than farmer's lung disease and pigeon breeder's lung disease), Pulmonary arteriovenous malformations in hereditary hemorrhagic telangiectasia (HHT), interstitial lung disease in systemic sclerosis, interstitial lung disease in rheumatoid arthritis, interstitial lung disease in idiopathic inflammatory myopathies (polymyositis, dermatomyositis, anti-synthetase syndrome), interstitial lung disease in Sjdgren syndrome, interstitial lung disease in mixed connective tissue disease (MCTD), interstitial lung disease in overlap syndromes, interstitial lung disease in undifferentiated connective tissue disease, Bronchiolitis obliterans (in non-transplanted patients), Infectious colitis, Ulcerative colitis, Crohn's disease, Ischemic colitis, Radiation colitis, Peptic ulcer, Intestinal cancer, Intestinal obstruction, Rheumatoid arthritis, Psoriatic arthritis, Hashimoto thyroiditis, Systemic lupus erythematosus, Multiple Sclerosis, Graves' Disease, Type 1 Diabetes Mellitus, Psoriasis, Ankylosing spondylitis, Scleroderma, Myositis, Gout, Antiphospholipid Antibody Syndrome (APS), Vasculitis, Dilated cardiomyopathy, Hypertrophic cardiomyopathy, Restrictive cardiomyopathy, Systolic heart failure, Diastolic heart failure (heart failure with preserved ejection fraction), Atrial Septal Defect, Atrioventricular Septal Defect, Coarctation of the Aorta, Double-outlet Right Ventricle, d-Transposition of the Great Arteries, Ebstein Anomaly, Hypoplastic Left Heart Syndrome, Interrupted Aortic Arch, Pulmonary Atresia, Single Ventricle, Tetralogy of Fallot, Total Anomalous Pulmonary Venous Return, Tricuspid Atresia, Truncus Arteriosus, Ventricular Septal Defect, Polycystic kidney disease, Diabetes Insipidus, Goodpasture's Disease, IgA Vasculitis, IgA Nephropathy, Lupus Nephritis, Adult Nephrotic Syndrome, Childhood Nephrotic Syndrome, Hemolytic Uremic Syndrome, Medullary Sponge Kidney, Kidney dysplasia, Renal artery stenosis, Renovascular hypertension, Renal tubular acidosis, Alport syndrome, Wenger's granulomatosis, Alagille syndrome, Cystinosis, Fabry disease, Focal segmental glomerulosclerosis (FSGS), Glomerulonephritis, aHUS (atypical hemolytic uremic syndrome), Hemolytic uremic syndrome (HUS), Henoch-Schonlein purpura, IgA nephropathy (Berger's disease), Interstitial nephritis, Minimal change disease, Nephrotic syndrome, Thrombotic thrombocytopenic purpura (TTP), Granulomatosis with polyangiitis (GPA), Adult Still's disease, Agammaglobulinemia, Alopecia areata, Autoimmune angioedema, Autoimmune dysautonomia, Autoimmune encephalomyelitis, Autoimmune hepatitis, Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Bullous pemphigoid, Celiac disease, Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Coxsackie myocarditis, CREST syndrome, Dermatitis herpetiformis, Dermatomyositis, Devic's disease (neuromyelitis optica), Discoid lupus, Eosinophilic esophagitis (EoE), Eosinophilic fasciitis, Erythema nodosum, Essential mixed cryoglobulinemia, Giant cell arteritis (temporal arteritis), Giant cell myocarditis, Granulomatosis with Polyangiitis, Guillain-Barre syndrome, Hashimoto's thyroiditis, Henoch-Schonlein purpura (HSP), Herpes gestationis or pemphigoid gestationis (PG), Hypogammalglobulinemia, IgG4-related sclerosing disease, Immune thrombocytopenic purpura (ITP), Inclusion body myositis (IBM), Lambert-Eaton syndrome, Leukocytoclastic vasculitis, Linear IgA disease (LAD), Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Primary biliary cirrhosis, Primary sclerosing cholangitis, Progesterone dermatitis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Thyroid eye disease (TED), Alagille Syndrome, Alcohol-Related Liver Disease, Autoimmune Hepatitis, Biliary Atresia, Cirrhosis, Lysosomal Acid Lipase Deficiency (LAL-D), Newborn Jaundice, Non-Alcoholic Fatty Liver Disease, Non-Alcoholic Steatohepatitis, Primary Biliary Cholangitis (PBC), Progressive Familial Intrahepatic Cholestasis (PFIC), Surgical scars, Hypertrophic scars, Acute Lymphoblastic Leukemia (ALL), Acute Myeloid Leukemia (AML), Adrenocortical Carcinoma, Kaposi Sarcoma (Soft Tissue Sarcoma), AIDS-Related Lymphoma (Lymphoma), Primary CNS Lymphoma (Lymphoma), Anal Cancer, Childhood astrocytomas, Atypical teratoid/rhabdoid tumor, Basal cell carcinoma of the skin, Bile duct cancer, Bladder cancer, Ewing Sarcoma, Osteosarcoma, Malignant fibrous histiocytoma, Brain tumor, Breast cancer, Bronchial tumor, Burkitt Lymphoma, Carcinoid Tumor (Gastrointestinal), Cardiac (Heart) Tumors, Childhood, Medulloblastoma, Germ Cell Tumor, Primary CNS Lymphoma, Cervical Cancer, Cholangiocarcinoma, Chordoma, Chronic Lymphocytic Leukemia (CLL), Chronic Myelogenous Leukemia (CML), Chronic Myeloproliferative Neoplasms, Colorectal Cancer, Craniopharyngioma, Cutaneous T-Cell Lymphoma, Ductal Carcinoma In Situ (DCIS), Endometrial Cancer (Uterine Cancer), Ependymoma, Esophageal Cancer, Esthesioneuroblastoma (Head and Neck Cancer), Retinoblastoma, Fallopian Tube Cancer, Gallbladder Cancer, Gastric (Stomach) Cancer, Gastrointestinal Carcinoid Tumor, Gastrointestinal Stromal Tumors (GIST), Germ Cell Tumors, Childhood Extracranial Germ Cell Tumors, Extragonadal Germ Cell Tumors, Ovarian Germ Cell Tumors, Testicular Cancer, Gestational Trophoblastic Disease, Hairy Cell Leukemia, Head and Neck Cancer, Heart Tumor, HepatocellularCancer, Histiocytosis, Langerhans Cell, Hodgkin Lymphoma, Hypopharyngeal Cancer (Head and Neck Cancer), Intraocular Melanoma, Islet Cell Tumors, Kidney (Renal Cell) Cancer, Langerhans Cell Histiocytosis, Laryngeal Cancer (Head and Neck Cancer), Lip and Oral Cavity Cancer, Lung Cancer (Non-Small Cell, Small Cell, Pleuropulmonary Blastoma, and Tracheobronchial Tumor), Male Breast Cancer, Melanoma, Melanoma, Intraocular (Eye), Meningioma, Merkel Cell Carcinoma (Skin Cancer), Mesothelioma, Malignant, Metastatic Squamous Neck Cancer with Occult Primary (Head and Neck Cancer), Midline Tract Carcinoma, Multiple Endocrine Neoplasia Syndromes, Multiple Myeloma/Plasma Cell Neoplasms, Mycosis Fungoides, Myelodysplastic Syndromes, Myelodysplastic/Myeloproliferative Neoplasms, Neuroblastoma, Non-Hodgkin Lymphoma, Non-Small Cell Lung Cancer, Oral Cancer, Lip and Oral Cavity Cancer and Oropharyngeal Cancer (Head and Neck Cancer), Osteosarcoma and Undifferentiated Pleomorphic Sarcoma of Bone Treatment, Ovarian Cancer, Pancreatic Cancer, Pancreatic Neuroendocrine Tumors, Papillomatosis, Paraganglioma, Parathyroid Cancer, Penile Cancer, Pharyngeal Cancer, Pheochromocytoma, Pituitary Tumor, Plasma Cell Neoplasm/Multiple Myeloma, Pleuropulmonary Blastoma, Pregnancy and Breast Cancer, Primary Central Nervous System (CNS) Lymphoma, Primary Peritoneal Cancer, Prostate Cancer, Rectal Cancer, Renal Cell (Kidney) Cancer, Retinoblastoma, Ewing Sarcoma (Bone Cancer), Soft Tissue Sarcoma, Sezary Syndrome, Skin Cancer, Small Cell Lung Cancer, Small Intestine Cancer, Soft Tissue Sarcoma, Squamous Cell Carcinoma of the Skin, Squamous Neck Cancer with Occult Primary, Metastatic (Head and Neck Cancer), Stomach (Gastric) Cancer, T-Cell Lymphoma, Cutaneous—see Lymphoma (Mycosis Fungoides and Sbzary Syndrome), Testicular Cancer, Nasopharyngeal Cancer, Oropharyngeal Cancer, Hypopharyngeal Cancer, Thymoma and Thymic Carcinoma, Thyroid Cancer, Tracheobronchial Tumors, Transitional Cell Cancer of the Renal Pelvis and Ureter, Urethral Cancer, Uterine Cancer, Endometrial, Uterine Sarcoma, Vaginal Cancer, Vulvar Cancer, and Wilms Tumor.

In illustrative embodiments, the disease is selected from the group consisting of brain tumor, gastric cancer, colorectal cancer, mesothelioma, non-small cell lung cancer, meningioma, head and neck cancer, and soft tissue sarcoma.

The compounds of the present disclosure are efficacious as single therapeutic agents, such as for the treatment of YAP-addicted cancers including glioblastoma, Head and neck squamous cell carcinoma, breast cancers, non-small cell lung cancer, melanoma, hepatocellular carcinoma, pancreatic adenocarcinoma, colorectal cancer, and prostate adenocarcinoma. In accordance with the methods described herein, a compound of the present disclosure can be administered with at least one additional chemotherapeutic agent. The additional chemotherapeutic agent can be administered prior to, concomitantly with, or after administration of the compound of the present disclosure. The compounds are therefore useful as combinatorial therapeutic agents, as TEAD inhibition has been shown to result in synergistic efficacy when co-treated with other targeted therapies (e.g., EGFR inhibitors, MEK inhibitors, and ERK1/2 inhibitors). ¹⁵ Because YAP controls PD-L1 levels and reprograms the tumor microenvironment, inhibitors of TEAD are useful as co-therapies with checkpoint inhibitors such as PD-L1, PD1, and CTLA4 targeting antibodies.

In various embodiments, the additional chemotherapeutic agent is selected from a B-RAF inhibitor, epidermal growth factor receptor (EGFR) inhibitor, MEK inhibitor, and immune check point inhibitor. Examples of a B-RAF inhibitor include vemurafenib, dabrafenib, and encorafenib. Examples of a MEK inhibitor include trametinib, cobimetinib, selumetinib, and binimetinib. In addition, examples of the EGFR inhibitor include erlotinib, osimertinib, neratinib, gefitinib, cetuximab, panitumumab, dacomitinib, lapatinib, necitumumab, mobocertinib, and vandetanib.

In various embodiments, the immune check point inhibitor is selected from the group consisting of a PD-1 inhibitor and a PD-L1 inhibitor. Examples of a PD-1 inhibitor include pembrolizumab, nivolumab, and cemiplimab. Examples of a PD-L1 inhibitor include atezolizumab, avelumab, and durvalumab.

### EXAMPLES

Additional embodiments of the disclosure reside in specific examples and data described in more detail herein.

Synthesis of Compounds. The compounds of the present disclosure are made according to, and by adaptation of, the following exemplary procedures.

### Example 1. Synthesis of 4-(3-((4-(trifluoromethyl)phenyl)amino)pyrazin-2-yl)piperazine-1-carbonitrile

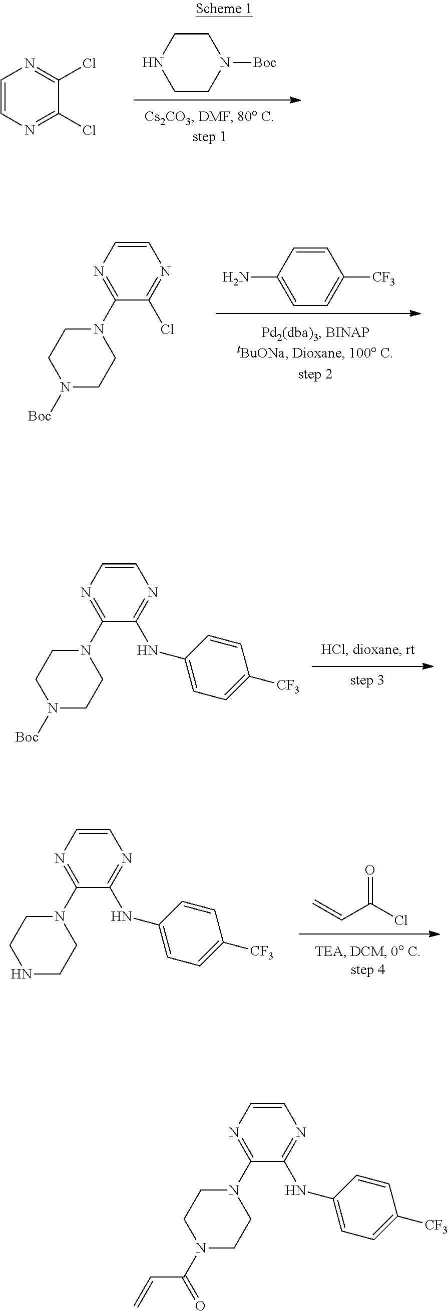

### Step 1: Synthesis of tert-butyl 4-(3-chloropyrazin-2-yl)piperazine-1-carboxylate

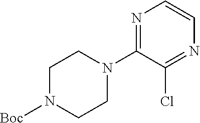 [SMILES: CC(C)(C)OC(=O)N1CCN(c2nccnc2Cl)CC1]

To a stirred mixture of 2,3-dichloropyrazine (1.00 g, 6.71 mmol, 1.00 equiv) and tert-butyl piperazine-1-carboxylate (1.00 g, 5.37 mmol, 0.80 equiv) in DMF (10.00 mL) was added Cs₂CO₃ (4.37 g, 13.43 mmol, 2.00 equiv) in portions at room temperature. The resulting mixture was stirred for 4 h at 80° C. The reaction was monitored by LCMS. The mixture was quenched with 20.00 mL H₂O and extracted with ethyl acetate (20.00 mL×3). The combined organic layers were washed with brine (50.00 mL), dried over anhydrous Na₂SO₄. After filtration, the filtrate was concentrated under reduced pressure. The residue was purified by silica gel column chromatography, eluted with PE: EA (5:1) to afford tert-butyl 4-(3-chloropyrazin-2-yl)piperazine-1-carboxylate (1.00 g, 49.86% yield) as a yellow solid. LCMS:(Positive, ES, m/z): 299.2

### Step 2: Synthesis of Tert-butyl 4-(3-((4-(trifluoromethyl)phenyl)amino)pyrazin-2-yl)piperazine-1-carboxylate

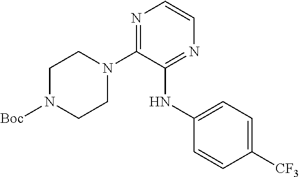 [SMILES: CC(C)(C)OC(=O)N1CCN(c2nccnc2Nc2ccc(C(F)(F)F)cc2)CC1]

To a stirred mixture of tert-butyl 4-(3-chloropyrazin-2-yl)piperazine-1-carboxylate (0.90 g, 3.01 mmol, 1.00 equiv) and P-trifluoromethylaniline (0.58 g, 3.61 mmol, 1.20 equiv), XantPhos (0.14 g, 0.24 mmol, 0.08 equiv) and CS₂CO₃ (1.96 g, 6.02 mmol, 2 equiv) in toluene (10.00 mL) was added Pd₂(dba)₃ (0.11 g, 0.12 mmol, 0.04 equiv) in portions at room temperature under nitrogen atmosphere. The resulting mixture was stirred for 4 h at 100° C. under nitrogen atmosphere. The mixture was allowed to cool down to room temperature and concentrated under reduced pressure. The residue was purified by silica gel column chromatography, eluted with PE:EA (4:1) to afford tert-butyl 4-(3-{[4-(trifluoromethyl)phenyl]amino}pyrazin-2-yl)piperazine-1-carboxylate (0.8 g, 62.72% yield) as a yellow oil. LCMS:(Positive, ES, m/z): 424.2

### Step 3: Synthesis of 3-(piperazin-1-yl)-N-(4-(trifluoromethyl)phenyl)pyrazin-2-amine

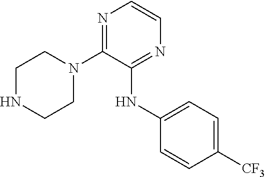 [SMILES: FC(F)(F)c1ccc(Nc2nccnc2N2CCNCC2)cc1]

To a stirred mixture of tert-butyl 4-(3-{[4-(trifluoromethyl)phenyl]amino}pyrazin-2-yl)piperazine-1-carboxylate (0.50 g, 1.18 mmol, 1.00 equiv) in DCM (2.00 mL) was added HCl(gas) in 1,4-dioxane (1.48 mL, 5.91 mmol, 5.00 equiv) at 0° C. The resulting mixture was stirred for 2 h at room temperature. The resulting mixture was concentrated under reduced pressure to afford 3-(piperazin-1-yl)-N-[4-(trifluoromethyl)phenyl]pyrazin-2-amine (0.30 g, 78.58% yield) as a yellow oil. LCMS:(Negative, ES, m/z): 322.1

### Step 4: Synthesis of 1-(4-(3-((4-(trifluoromethyl)phenyl)amino)pyrazin-2-yl)piperazin-1-yl)prop-2-en-1-one

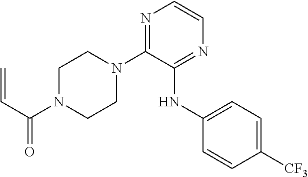 [SMILES: C=CC(=O)N1CCN(c2nccnc2Nc2ccc(C(F)(F)F)cc2)CC1]

Into a 10 ml bottle were added 3-(piperazin-1-yl)-N-[4-(trifluoromethyl)phenyl]pyrazin-2-amine (0.30 g, 0.93 mmol, 1.00 equiv) and TEA (0.28 g, 2.78 mmol, 3.00 equiv) in DCM (5.00 mL) at 0° C. To the above mixture was added acryloyl chloride (0.08 g, 0.93 mmol, 1.00 equiv). The resulting mixture was stirred for additional 2 h at room temperature. The reaction was monitored by LCMS. The resulting mixture was added 50.00 mL H₂O and extracted with ethyl acetate (50.00 mL×3). The combined organic layers were washed with brine (100.00 mL), dried over anhydrous Na₂SO₄. After filtration, the filtrate was concentrated under reduced pressure. The crude product was purified by (Column: Welch Xtimate C18 ExRS, 250 mm, 10 μm; Mobile Phase A: Water (0.05% NH₃·H₂O), Mobile Phase B: ACN; Flow rate: 90 mL/min; Gradient: 90% B to 90% B in 10 min, 90% B; Wave Length: 254 nm; RT1(min): 7; Number Of Runs: 5) to afford 1-[4-(3-{[4-(trifluoromethyl)phenyl]amino}pyrazin-2-yl)piperazin-1-yl]prop-2-en-1-one (0.06 g, 17.14% yield) as white solid. LCMS: (Positive, ES, m/z): 349.2.¹H NMR (400 MHz, DMSO-d₆): δ 8.60 (s, 1H), 7.92-7.87 (m, 3H), 7.84-7.83 (m, 1H), 7.65-7.63 (m, 2H), 6.78-6.81 (m, 1H), 6.17-6.12 (m, 1H), 5.72-5.69 (m, 1H), 3.80-3.76 (m, 4H), 3.16-3.13 (m, 4H).

### Example 2. Synthesis of 4-(3-{[4-(trifluoromethyl)phenyl]amino}pyrazin-2-yl)piperazine-1-carbonitrile

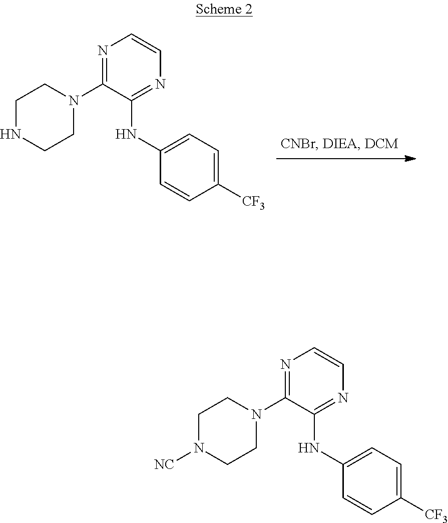 [SMILES: FC(F)(F)c1ccc(Nc2nccnc2N2CCNCC2)cc1 ; N#CN1CCN(c2nccnc2Nc2ccc(C(F)(F)F)cc2)CC1]

Into a 10 ml bottle were added 3-(piperazin-1-yl)-N-[4-(trifluoromethyl)phenyl]pyrazin-2-amine (0.20 g, 0.62 mmol, 1.00 equiv) and DIEA (0.24 g, 1.86 mmol, 3.00 equiv) in DCM (2.00 mL) at 0° C. To the above mixture was added BrCN (0.07 g, 0.62 mmol, 1.00 equiv). The resulting mixture was stirred for additional 2 h at room temperature. The reaction was monitored by LCMS. The resulting mixture was added 50.00 mL H₂O and extracted with ethyl acetate (50.00 mL×3). The combined organic layers were washed with brine (100.00 mL), dried over anhydrous Na₂SO₄. After filtration, the filtrate was concentrated under reduced pressure. The crude product was purified by (Column: Welch Xtimate C18 ExRS, 250 mm, 10 μm; Mobile Phase A: Water (0.05% NH₃·H₂O), Mobile Phase B: ACN; Flow rate: 90 mL/min; Gradient: 90% B to 90% B in 10 min, 90% B; Wave Length: 254 nm; RT1(min): 7; Number Of Runs: 5) to afford 4-(3-{[4-(trifluoromethyl)phenyl]amino}pyrazin-2-yl)piperazine-1-carbonitrile (65.00 mg, 30.17% yield) as white solid. LCMS: (Positive, ES, m/z): 349.2.¹HNMR (400 MHz, DMSO-d₆): δ 8.55 (s, 1H), 7.92-7.89 (m, 3H), 7.86-7.85 (m, 1H), 7.66-7.63 (m, 2H), 3.47-3.40 (m, 4H), 3.22-3.19 (m, 4H).

### Example 3. Synthesis ofN-(1-(3-((4-(trifluoromethyl)phenyl)amino)pyrazin-2-yl)azetidin-3-yl) acrylamide

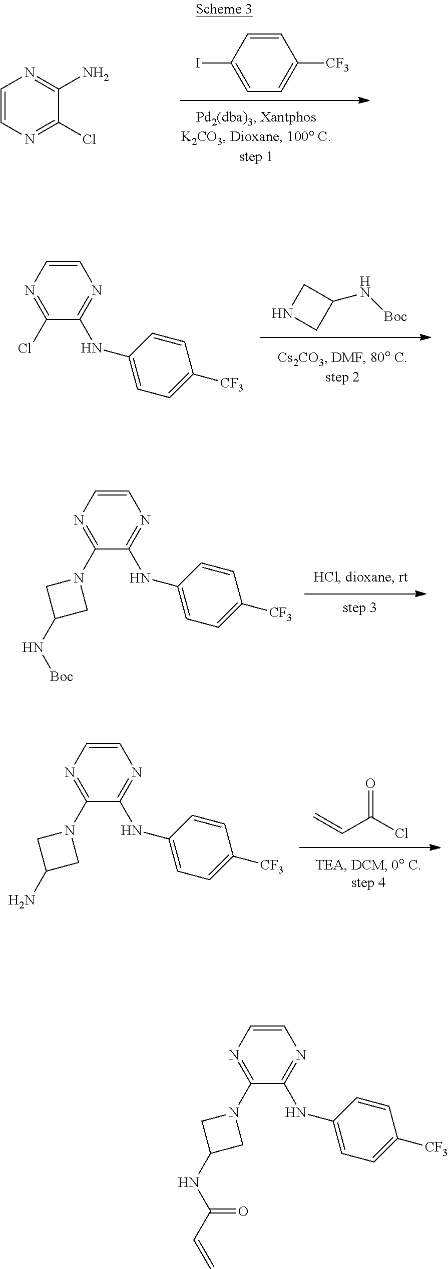

### Step 1: Synthesis of 3-chloro-N-[4-(trifluoromethyl)phenyl]pyrazin-2-amine

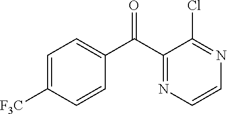 [SMILES: O=C(c1ccc(C(F)(F)F)cc1)c1nccnc1Cl]

To a solution of 3-chloropyrazin-2-amine (4.00 g, 30.88 mmol, 1.00 equiv), 1-iodo-4-(trifluoromethyl)benzene (8.40 g, 30.88 mmol, 1.00 equiv), Xtanphos (1.79 g, 3.09 mmol, 0.10 equiv) and Cs₂CO₃ (25.15 g, 77.19 mmol, 2.00 equiv) in toluene (20.00 mL) was added Pd₂(dba)₃ (2.83 g, 3.09 mmol, 0.10 equiv). The resulting mixture was stirred for 3 h at 90° C. under N₂ atmosphere. The mixture was diluted with EA (150.00 mL), washed with H₂O (3×50.00 mL), dried over anhydrous Na₂SO₄. After filtration, the filtrate was concentrated under reduced pressure. The residue was purified by silica gel column chromatography, eluted with PE:EA (5:1) to afford 3-chloro-N-[4-(trifluoromethyl)phenyl]pyrazin-2-amine (3.50 g, 35.51%) as a yellow solid. (ES, m/z):[M+H]⁺=274. ¹H NMR (400 MHz, DMSO-d₆): δ 9.17 (s, 1H), 8.22 (s, 1H), 7.95-7.93 (m, 3H), 7.68-7.66 (m, 2H).

### Step 2: Synthesis of Tert-butyl (1-(3-((4-(trifluoromethyl)phenyl)amino)pyrazin-2-yl)azetidin-3-yl)carbamate

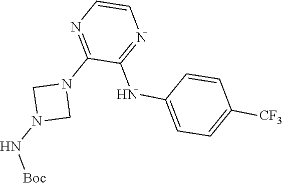 [SMILES: CC(C)(C)OC(=O)NN1CN(c2nccnc2Nc2ccc(C(F)(F)F)cc2)C1]

To a stirred mixture of 3-chloro-N-[4-(trifluoromethyl)phenyl]pyrazin-2-amine (200 mg, 0.73 mmol, 1.00 equiv) and tert-butyl azetidin-3-ylcarbamate (151 mg, 0.88 mmol, 1.2 equiv) in DMF (10.00 mL) were added Cs₂CO₃ (476.3 mg, 1.46 mmol, 2.00 equiv) in portions at room temperature. The resulting mixture was stirred overnight at 80° C. The reaction was monitored by LCMS. The mixture was quenched with 20.00 mL H₂O and extracted with ethyl acetate (20.00 mL×3). The combined organic layers were washed with brine (50.00 mL), dried over anhydrous Na₂SO₄. After filtration, the filtrate was concentrated under reduced pressure. The residue was purified by silica gel column chromatography, eluted with DCM (100%) to afford tert-butyl (1-(3-((4-(trifluoromethyl)phenyl)amino)pyrazin-2-yl)azetidin-3-yl)carbamate (250 mg, 83.55% yield) as a yellow solid.

### Step 3: Synthesis of 3-(3-aminoazetidin-1-yl)-N-(4-(trifluoromethyl)phenyl)pyrazin-2-amine

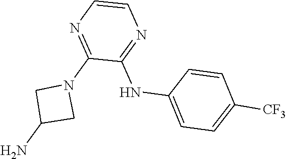 [SMILES: NC1CN(c2nccnc2Nc2ccc(C(F)(F)F)cc2)C1]

To a stirred mixture of tert-butyl 4-(3-(4-(trifluoromethyl)phenyl)pyrazin-2-yl)piperazine-1-carboxylate (250 mg, 0.0.61 mmol, 1.00 equiv) in Dioxane (2.00 mL) were added HCl(gas) in 1,4-dioxane (3.05 mL, 12.21 mmol, 20.00 equiv) at 0° C. The resulting mixture was stirred for 2 h at room temperature. The resulting mixture was concentrated under reduced pressure to afford 3-(3-aminoazetidin-1-yl)-N-(4-(trifluoromethyl)phenyl)pyrazin-2-amine (170 mg, 90.01% yield) as a yellow oil.

### Step 4: Synthesis of N-(1-(3-((4-(trifluoromethyl)phenyl)amino)pyrazin-2-yl)azetidin-3-yl) acrylamide

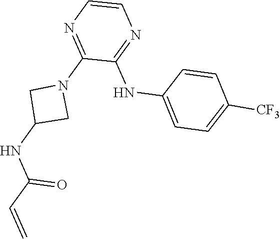 [SMILES: C=CC(=O)NC1CN(c2nccnc2Nc2ccc(C(F)(F)F)cc2)C1]

3-(3-aminoazetidin-1-yl)-N-(4-(trifluoromethyl)phenyl)pyrazin-2-amine (100 mg, 0.32 mmol, 1.00 equiv) and TEA (134 mmL, 0.81 mmol, 2.50 equiv) in DCM (2.00 mL) at 0° C. To the above mixture was added acryloyl chloride (32 mmL, 0.39 mmol, 1.20 equiv). The resulting mixture was stirred for additional 0.5 h at 0° C. The reaction was monitored by LCMS. The resulting mixture was added 50.00 mL H₂O and extracted with ethyl acetate (50.00 mL×3). The combined organic layers were washed with brine (100.00 mL), dried over anhydrous Na₂SO₄. After filtration, the filtrate was concentrated under reduced pressure. The crude product was purified by silica gel column chromatography, eluted with DCM:EA (4:6) to afford N-(1-(3-((4-(trifluoromethyl)phenyl)amino)pyrazin-2-yl)azetidin-3-yl) acrylamide (53 mg, 45.12% yield) as white solid. ¹H NMR (400 MHz, DMSO) δ 10.86-10.30 (m, 1H), 8.59-8.31 (m, 1H), 8.17-7.82 (m, 2H), 7.77-7.59 (m, 2H), 7.59-7.33 (m, 2H), 6.31-6.07 (m, 2H), 5.71-5.59 (m, 1H), 4.67 (s, 1H), 4.48-4.13 (m, 2H), 3.85-3.50 (m, 3H).

### Example 4: Synthesis of N-((1r,3r)-3-((3-((4-(trifluoromethyl)phenyl)amino)pyrazin-2-yl)amino)cyclobutyl) acrylamide

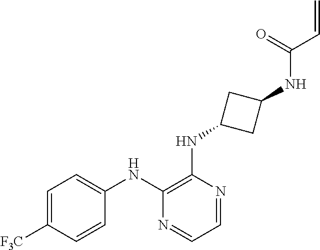 [SMILES: C=CC(=O)N[C@H]1C[C@H](Nc2nccnc2Nc2ccc(C(F)(F)F)cc2)C1]

Following the general procedure in scheme 3, tert-butyl ((1r,3r)-3-aminocyclobutyl)carbamate was used in step 2. Offered N-((1r,3r)-3-((3-((4-(trifluoromethyl)phenyl)amino)pyrazin-2-yl)amino)cyclobutyl) acrylamide as white solid. (Positive, ES, m/z): 378.19. ¹H NMR (400 MHz, DMSO) δ 8.73-8.54 (m, 2H), 7.88 (d, J=8.5 Hz, 2H), 7.65 (d, J=8.6 Hz, 2H), 7.56 (d, J=3.0 Hz, 1H), 7.43 (d, J=3.0 Hz, 1H), 7.00 (d, J=5.7 Hz, 1H), 6.24 (dd, J=17.1, 10.1 Hz, 1H), 6.10 (dd, J=17.0, 2.3 Hz, 1H), 5.61 (dd, J=10.0, 2.3 Hz, 1H), 4.51-4.39 (m, 2H), 2.34 (ddt, J=9.6, 5.5, 2.0 Hz, 4H).

### Example 5: Synthesis ofN-((ls,3s)-3-((3-((4-(trifluoromethyl)phenyl)amino)pyrazin-2-yl)amino)cyclobutyl) acrylamide

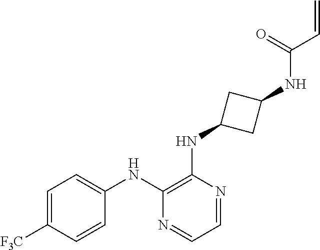 [SMILES: C=CC(=O)N[C@H]1C[C@@H](Nc2nccnc2Nc2ccc(C(F)(F)F)cc2)C1]

Following the general procedure in scheme 3, tert-butyl ((1s,3s)-3-aminocyclobutyl)carbamate was used in step 2. Offered N-((1 s,3s)-3-((3-((4-(trifluoromethyl)phenyl)amino)pyrazin-2-yl)amino)cyclobutyl) acrylamide as white solid. (Positive, ES, m/z): 378.24. ¹H NMR (400 MHz, DMSO) δ 8.64 (s, 1H), 8.42 (d, J=7.2 Hz, 1H), 7.88 (d, J=8.5 Hz, 2H), 7.65 (d, J=8.6 Hz, 2H), 7.57 (d, J=3.0 Hz, 1H), 7.42 (d, J=3.0 Hz, 1H), 6.93 (s, 1H), 6.27-6.05 (m, 2H), 5.60 (dd, J=9.9, 2.5 Hz, 1H), 4.06 (dd, J=16.6, 8.7 Hz, 2H), 2.84-2.72 (m, 2H), 1.98-1.84 (m, 2H).

### Example 6: Synthesis ofN-(2-(3-((4-(trifluoromethyl)phenyl)amino)pyrazin-2-yl)-2-azaspiro[3.3]heptan-6-yl)acrylamide

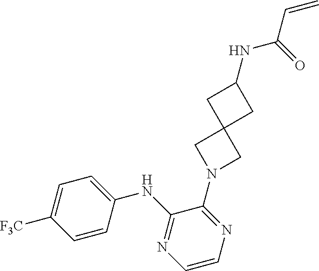 [SMILES: C=CC(=O)NC1CC2(C1)CN(c1nccnc1Nc1ccc(C(F)(F)F)cc1)C2]

Following the general procedure in scheme 3, tert-butyl (2-azaspiro[3.3]heptan-6-yl)carbamate was used in step 2. Offered N-(2-(3-((4-(trifluoromethyl)phenyl)amino)pyrazin-2-yl)-2-azaspiro[3.3]heptan-6-yl)acrylamide as white solid. (Positive, ES, m/z): 404.27. ¹H NMR (400 MHz, DMSO) δ 8.45 (s, 1H), 8.36 (d, J=7.4 Hz, 1H), 7.77-7.68 (m, 3H), 7.66-7.53 (m, 3H), 6.21-6.02 (m, 2H), 5.59 (dd, J=9.7, 2.6 Hz, 1H), 4.16 (d, J=17.6 Hz, 3H), 4.03 (s, 2H), 2.56-2.54 (m, 2H), 2.14 (td, J=8.8, 2.9 Hz, 2H).

### Example 7: Synthesis of N-(1-(3-((4-(trifluoromethyl)phenyl)amino)pyrazin-2-yl)pyrrolidin-3-yl)acrylamide

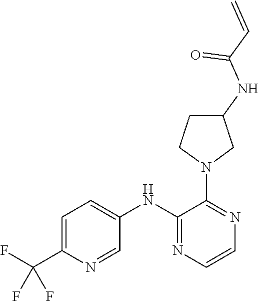 [SMILES: C=CC(=O)NC1CCN(c2nccnc2Nc2ccc(C(F)(F)F)nc2)C1]

Following the general procedure in scheme 3, tert-butyl pyrrolidin-3-ylcarbamate was used in step 2. Offered N-(1-(3-((4-(trifluoromethyl)phenyl)amino)pyrazin-2-yl)pyrrolidin-3-yl)acrylamide as white solid. (Positive, ES, m/z): 378.28. ¹H NMR (400 MHz, DMSO) δ 8.61 (s, 1H), 8.33 (d, J=6.5 Hz, 1H), 7.76 (d, J=2.7 Hz, 1H), 7.69-7.49 (m, 5H), 6.21 (dd, J=17.1, 10.0 Hz, 1H), 6.08 (dd, J=17.1, 2.3 Hz, 1H), 5.58 (dd, J=10.0, 2.3 Hz, 1H), 4.35 (q, J=6.1 Hz, 1H), 3.74 (dd, J=10.7, 6.5 Hz, 1H), 3.56 (d, J=6.9 Hz, 2H), 3.31 (dd, J=10.7, 5.3 Hz, 1H), 2.16 (dd, J=12.6, 6.4 Hz, 1H), 1.86 (dd, J=12.5, 6.2 Hz, 1H).

### Example 8: Synthesis of 1-(3-((3-((4-(trifluoromethyl)phenyl)amino)pyrazin-2-yl)amino)pyrrolidin-1-yl)prop-2-en-1-one

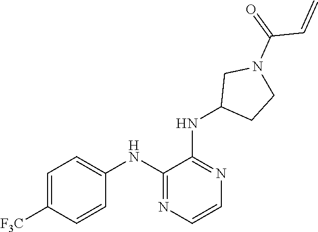 [SMILES: C=CC(=O)N1CCC(Nc2nccnc2Nc2ccc(C(F)(F)F)cc2)C1]

Following the general procedure in scheme 3, tert-butyl 3-aminopyrrolidine-1-carboxylate was used in step 2. Offered N-(1-(3-((4-(trifluoromethyl)phenyl)amino)pyrazin-2-yl)pyrrolidin-3-yl)acrylamide as white solid. (Positive, ES, m/z): 378.28. ¹H NMR (400 MHz, DMSO) δ 8.61 (s, 1H), 8.33 (d, J=6.5 Hz, 1H), 7.76 (d, J=2.7 Hz, 1H), 7.69-7.49 (m, 5H), 6.21 (dd, J=17.1, 10.0 Hz, 1H), 6.08 (dd, J=17.1, 2.3 Hz, 1H), 5.58 (dd, J=10.0, 2.3 Hz, 1H), 4.35 (q, J=6.1 Hz, 1H), 3.74 (dd, J=10.7, 6.5 Hz, IH), 3.56 (d, J=6.9 Hz, 2H), 3.31 (dd, J=10.7, 5.3 Hz, 1H), 2.16 (dd, J=12.6, 6.4 Hz, 1H), 1.86 (dd, J=12.5, 6.2 Hz, 1H).

### Example 9: Synthesis of N-(1-(3-((4-(trifluoromethyl)phenyl)amino)pyrazin-2-yl)piperidin-4-yl)acrylamide

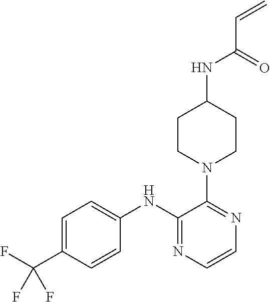 [SMILES: C=CC(=O)NC1CCN(c2nccnc2Nc2ccc(C(F)(F)F)cc2)CC1]

Following the general procedure in scheme 3, tert-butyl piperidin-4-ylcarbamate was used in step 2. Offered N-(1-(3-((4-(trifluoromethyl)phenyl)amino)pyrazin-2-yl)piperidin-4-yl)acrylamide as white solid. (Positive, ES, m/z): 392.26. ¹H NMR (400 MHz, DMSO) δ 8.52 (s, 1H), 8.12 (d, J=7.4 Hz, 1H), 7.88 (d, J=8.5 Hz, 2H), 7.84-7.76 (m, 2H), 7.62 (d, J=8.6 Hz, 2H), 6.26 (dd, J=17.1, 10.1 Hz, 1H), 6.09 (dd, J=17.1, 2.3 Hz, 1H), 5.57 (dd, J=10.1, 2.3 Hz, IH), 3.84 (d, J=11.2 Hz, IH), 3.51 (d, J=13.0 Hz, 2H), 3.00-2.84 (m, 2H), 1.88 (d, J=12.3 Hz, 2H), 1.77-1.65 (m, 2H).

### Example 10: Synthesis of N-((1-(3-((4-(trifluoromethyl)phenyl)amino)pyrazin-2-yl)piperidin-3-yl)methyl)acrylamide

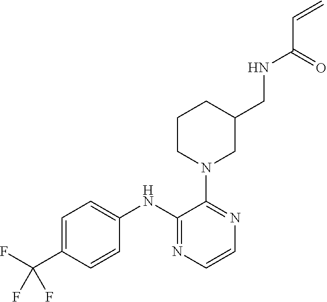 [SMILES: C=CC(=O)NCC1CCCN(c2nccnc2Nc2ccc(C(F)(F)F)cc2)C1]

Following the general procedure in scheme 3, tert-butyl (piperidin-3-ylmethyl)carbamate was used in step 2. Offered N-((1-(3-((4-(trifluoromethyl)phenyl)amino)pyrazin-2-yl)piperidin-3-yl)methyl) acrylamide as white solid. (Positive, ES, m/z): 406.32. ¹H NMR (400 MHz, DMSO) δ 8.33 (s, 1H), 8.18 (t, J=5.9 Hz, 1H), 7.88-7.76 (m, 4H), 7.59 (d, J=8.7 Hz, 2H), 6.13 (dd, J=17.1, 10.0 Hz, 1H), 6.00 (dd, J=17.1, 2.3 Hz, IH), 5.49 (dd, J=10.0, 2.3 Hz, 1H), 3.77-3.53 (m, 4H), 3.18-2.97 (m, 2H), 1.99 (s, 1H), 1.83-1.65 (m, 3H), 1.24-1.01 (m, 1H).

### Example 11: Synthesis of 1-(5-(3-((4-(trifluoromethyl)phenyl)amino)pyrazin-2-yl)-2,5-diazabicyclo[2.2.1]heptan-2-yl)prop-2-en-1-one

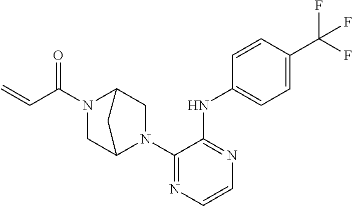 [SMILES: C=CC(=O)N1CC2CC1CN2c1nccnc1Nc1ccc(C(F)(F)F)cc1]

Following the general procedure in scheme 3, tert-butyl 2,5-diazabicyclo[2.2.1]heptane-2-carboxylate was used in step 2. Offered 1-(5-(3-((4-(trifluoromethyl)phenyl)amino)pyrazin-2-yl)-2,5-diazabicyclo[2.2.1]heptan-2-yl)prop-2-en-1-one as white solid. (Positive, ES, m/z): 390.24. ¹H NMR (400 MHz, DMSO) δ 8.46 (s, 1H), 7.75 (d, J=2.8 Hz, 1H), 7.67 (dd, J=3.8, 2.8 Hz, 1H), 7.61-7.53 (m, 4H), 6.75-6.31 (m, 1H), 6.10 (ddd, J=16.8, 9.3, 2.4 Hz, 1H), 5.63 (ddd, J=14.3, 10.2, 2.4 Hz, 1H), 4.91-4.67 (m, 2H), 4.04-3.61 (m, 4H), 1.98-1.85 (m, 2H).

### Example 12: Synthesis of 1-((1R,5S)-3-(3-((4-(trifluoromethyl)phenyl)amino)pyrazin-2-yl)-3,8-diazabicyclo[3.2.1]octan-8-yl)prop-2-en-1-one

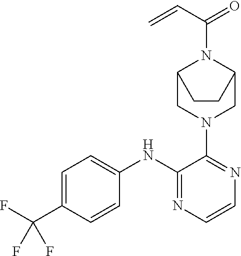 [SMILES: C=CC(=O)N1C2CCC1CN(c1nccnc1Nc1ccc(C(F)(F)F)cc1)C2]

Following the general procedure in scheme 3, tert-butyl (1R,5S)-3,8-diazabicyclo[3.2.1]octane-8-carboxylate was used in step 2. Offered 1-((1R,5S)-3-(3-((4-(trifluoromethyl)phenyl)amino)pyrazin-2-yl)-3,8-diazabicyclo[3.2.1]octan-8-yl)prop-2-en-1-one as white solid. (Positive, ES, m/z): 404.30. ¹H NMR (400 MHz, DMSO) δ 8.33 (s, 1H), 7.87 (q, J=2.7 Hz, 2H), 7.72-7.54 (m, 4H), 6.74 (dd, J=16.7, 10.4 Hz, 1H), 6.19 (dd, J=16.7, 2.4 Hz, 1H), 5.71 (dd, J=10.3, 2.4 Hz, 1H), 4.61 (d, J=7.6 Hz, 2H), 3.45 (d, J=12.0 Hz, 2H), 2.97 (dd, J=17.1, 11.9 Hz, 2H), 2.03-1.68 (m, 4H).

### Example 13: Synthesis of (R)-1-(2-methyl-4-(3-((4-(trifluoromethyl)phenyl)amino)pyrazin-2-yl)piperazin-1-yl)prop-2-en-1-one

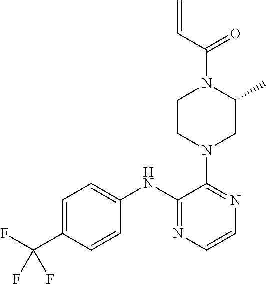 [SMILES: C=CC(=O)N1CCN(c2nccnc2Nc2ccc(C(F)(F)F)cc2)C[C@H]1C]

Following the general procedure in scheme 3, tert-butyl (R)-2-methylpiperazine-1-carboxylate was used in step 2. Offered (R)-1-(2-methyl-4-(3-((4-(trifluoromethyl)phenyl)amino)pyrazin-2-yl)piperazin-1-yl)prop-2-en-1-one as white solid. (Positive, ES, m/z): 392.1. ¹H NMR (400 MHz, DMSO) δ 8.34 (s, 1H), 7.89 (d, J=2.8 Hz, 1H), 7.87-7.79 (m, 3H), 7.65 (d, J=8.6 Hz, 2H), 6.81 (dd, J=16.7, 10.5 Hz, 1H), 6.14 (dd, J=16.6, 2.4 Hz, 1H), 5.70 (dd, J=10.5, 2.4 Hz, 1H), 3.56 (d, J=12.3 Hz, 1H), 3.43-3.30 (m, 4H), 2.93 (s, 1H), 2.68 (d,, =9.5 Hz, 1H), 1.42-1.22 (m, 3H).

### Example 14: Synthesis of (S)-1-(2-methyl-4-(3-((4-(trifluoromethyl)phenyl)amino)pyrazin-2-yl)piperazin-1-yl)prop-2-en-1-one

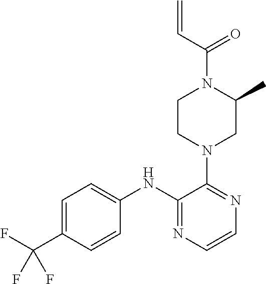 [SMILES: C=CC(=O)N1CCN(c2nccnc2Nc2ccc(C(F)(F)F)cc2)C[C@@H]1C]

Following the general procedure in scheme 3, tert-butyl (S)-2-methylpiperazine-1-carboxylate was used in step 2. Offered (S)-1-(2-methyl-4-(3-((4-(trifluoromethyl)phenyl)amino)pyrazin-2-yl)piperazin-1-yl)prop-2-en-1-one as white solid. (Positive, ES, m/z): 392.26. ¹H NMR (400 MHz, DMSO) δ 8.36 (s, 1H), 7.95-7.81 (m, 4H), 7.66 (d, J=8.6 Hz, 2H), 6.83 (dd, J=16.6, 10.5 Hz, 1H), 6.15 (dd, J=16.7, 2.5 Hz, 1H), 5.71 (dd, J=10.5, 2.4 Hz, 1H), 3.58 (d, J=12.5 Hz, 1H), 3.35 (s, 4H), 2.94 (s, 1H), 2.81-2.62 (m, 1H), 1.36 (d, J=6.7 Hz, 3H).

### Example 15: Synthesis of (R)-1-(2-(hydroxymethyl)-4-(3-((4-(trifluoromethyl)phenyl)amino)pyrazin-2-yl)piperazin-1-yl)prop-2-en-1-one

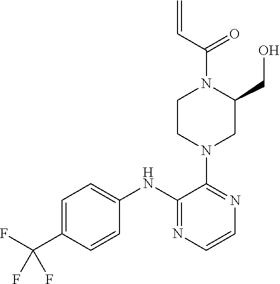 [SMILES: C=CC(=O)N1CCN(c2nccnc2Nc2ccc(C(F)(F)F)cc2)C[C@@H]1CO]

Following the general procedure in scheme 3, tert-butyl (R)-2-(hydroxymethyl)piperazine-1-carboxylate was used in step 2. Offered (R)-1-(2-(hydroxymethyl)-4-(3-((4-(trifluoromethyl)phenyl)amino)pyrazin-2-yl)piperazin-1-yl)prop-2-en-1-one as white solid. (Positive, ES, m/z): 408.38. ¹H NMR (400 MHz, DMSO) δ 8.99 (s, 1H), 8.08 (d, J=8.5 Hz, 2H), 7.79 (d, J=3.0 Hz, 1H), 7.68 (d, J=8.5 Hz, 2H), 7.61 (d, J=3.1 Hz, 1H), 6.85 (td, J=16.0, 10.4 Hz, 1H), 6.12 (dd, J=16.6, 2.4 Hz, 1H), 5.68 (t, J=8.8 Hz, 1H), 4.48-3.87 (m, 4H), 3.26-2.94 (m, 4H), 2.90-2.57 (m, 2H).

### Example 16: Synthesis of (S)-1-(2-(hydroxymethyl)-4-(3-((4-(trifluoromethyl)phenyl)amino)pyrazin-2-yl)piperazin-1-yl)prop-2-en-1-one

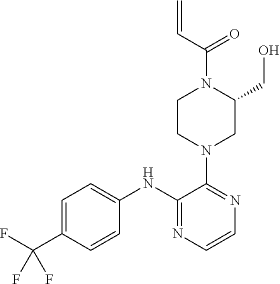 [SMILES: C=CC(=O)N1CCN(c2nccnc2Nc2ccc(C(F)(F)F)cc2)C[C@H]1CO]

Following the general procedure in scheme 3, tert-butyl (S)-2-(hydroxymethyl)piperazine-1-carboxylate was used in step 2. Offered (S)-1-(2-(hydroxymethyl)-4-(3-((4-(trifluoromethyl)phenyl)amino)pyrazin-2-yl)piperazin-1-yl)prop-2-en-1-one as white solid. (Positive, ES, m/z): 408.46. ¹H NMR (400 MHz, DMSO) δ 8.99 (s, 1H), 8.08 (d, J=8.5 Hz, 2H), 7.79 (d, J=3.0 Hz, 1H), 7.68 (d, J=8.5 Hz, 2H), 7.61 (d, J=3.0 Hz, 1H), 6.85 (td, J=16.0, 10.4 Hz, 1H), 6.12 (dd, J=16.7, 2.4 Hz, 1H), 5.68 (td, J=9.9, 2.5 Hz, 1H), 4.48-3.89 (m, 4H), 3.21-2.94 (m, 4H), 2.90-2.59 (m, 2H).

### Example 17: Synthesis of (S)-1-(2-(2-hydroxyethyl)-4-(3-((4-(trifluoromethyl)phenyl)amino)pyrazin-2-yl)piperazin-1-yl)prop-2-en-1-one

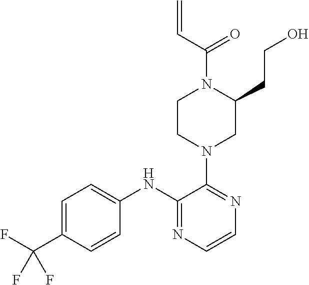 [SMILES: C=CC(=O)N1CCN(c2nccnc2Nc2ccc(C(F)(F)F)cc2)C[C@@H]1CCO]

Following the general procedure in scheme 3, tert-butyl (S)-2-(2-hydroxyethyl)piperazine-1-carboxylate was used in step 2. Offered (S)-1-(2-(2-hydroxyethyl)-4-(3-((4-(trifluoromethyl)phenyl)amino)pyrazin-2-yl)piperazin-1-yl)prop-2-en-1-one as white solid. (Positive, ES, m/z): 422.29. ¹H NMR (400 MHz, DMSO) δ 8.97 (s, 1H), 8.10 (d, J=8.4 Hz, 2H), 7.76 (d, J=3.0 Hz, 1H), 7.71-7.51 (m, 3H), 6.80 (dd, J=16.6, 10.4 Hz, 1H), 6.18-5.98 (m, 1H), 5.73-5.53 (m, 1H), 4.52 (dq, J=11.0, 5.7 Hz, 2H), 4.33-4.08 (m, 1H), 4.06-3.80 (m, 1H), 3.19-2.78 (m, 2H), 2.78-2.54 (m, 3H), 2.03-1.74 (m, 2H).

### Example 18. Synthesis of (R)-1-acryloyl-4-(3-((4-(trifluoromethyl)phenyl)amino)pyrazin-2-yl)piperazine-2-carboxamide

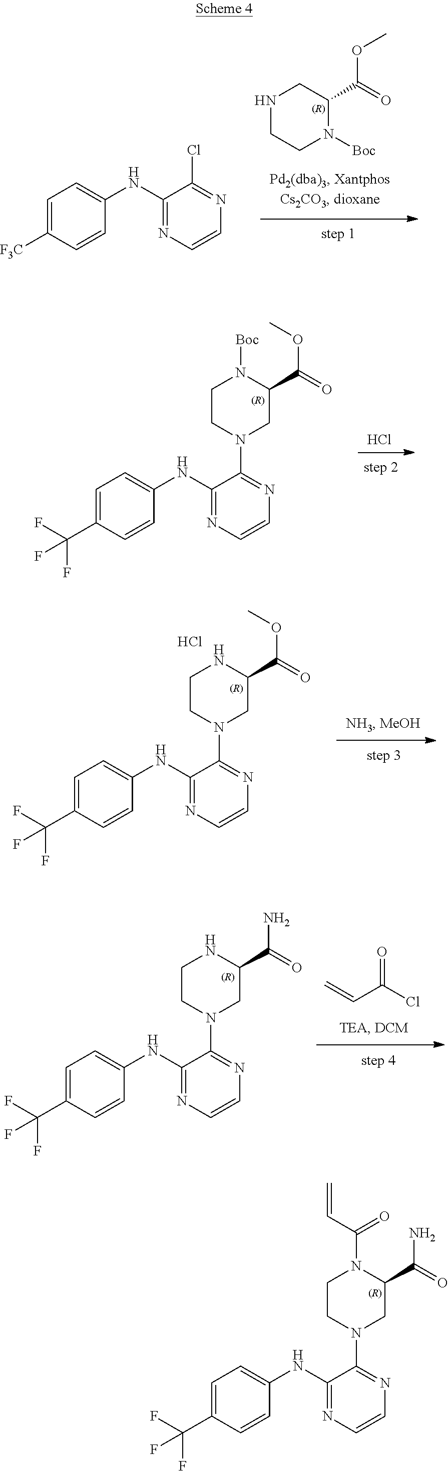

### Step 1: Synthesis of 1-(tert-butyl) 2-methyl (R)-4-(3-((4-(trifluoromethyl)phenyl)amino)pyrazin-2-yl)piperazine-1,2-dicarboxylate

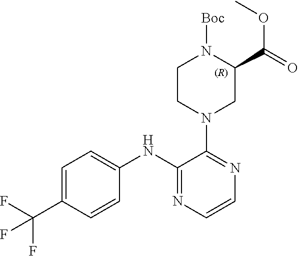

Into a 40 mL vial were added 3-chloro-N-[4-(trifluoromethyl)phenyl]pyrazin-2-amine (800 mg, 2.92 mmol, 1 equiv),1-tert-butyl 2-methyl (2R)-piperazine-1,2-dicarboxylate (749.91 mg, 3.07 mmol,1.05 equiv), toluene (10 mL), Pad(dppf)Cl₂ (80 mg) XantPhos (120 mg) and Cs₂CO₃ (2857.64 mg, 8.77 mmol, 3 equiv) at room temperature. The resulting mixture was stirred for 3 h at 90° C. under nitrogen atmosphere. Desired product could be detected by LCMS. The resulting mixture was concentrated under reduced pressure. The residue was purified by silica gel column chromatography, eluted with PE/EA (1:1) to afford 1-tert-butyl 2-methyl (2R)-4-(3-{[4-(trifluoromethyl)phenyl]amino}pyrazin-2-yl) piperazine-1,2-dicarboxylate (754.9 mg, 53.63%) as an off-white solid.

### Step 2: Synthesis of methyl (R)-4-(3-((4-(trifluoromethyl)phenyl)amino)pyrazin-2-yl)piperazine-2-carboxylate

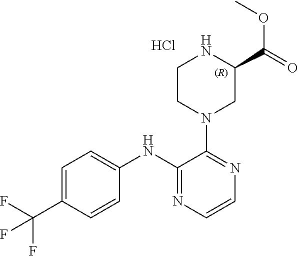

Into a 40 mL vial were added 1-tert-butyl 2-methyl (2R)-4-(3-{[4-(trifluoromethyl)phenyl]amino}pyrazin-2-yl) piperazine-1,2-dicarboxylate (600 mg,1.246 mmol, 1 equiv) DCM (9 mL) and HCl/dioxane (2 mL, 4M) at ° C. The resulting mixture was stirred for 1 h at room temperature. Desired product could be detected by LCMS. The resulting mixture was concentrated under reduced pressure. The crude product was used in the next step directly without further purification.

### Step 3: Synthesis of (R)-4-(3-((4-(trifluoromethyl)phenyl)amino)pyrazin-2-yl)piperazine-2-carboxamide

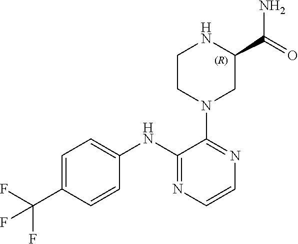

Into a 40 mL vial were added methyl (2R)-4-(3-{[4-(trifluoromethyl)phenyl]amino}pyrazin-2-yl) piperazine-2-carboxylate (400 mg, 0.787 mmol, 1 equiv) and NHI3(g) in MeOH (10 mL) at room temperature. The resulting mixture was stirred for 8 h at 100° C. Desired products could be detected by LCMS. The resulting mixture was concentrated under reduced pressure. The crude product was used in the next step directly without further purification.

### Step 4: Synthesis of (R)-1-acryloyl-4-(3-((4-(trifluoromethyl)phenyl)amino)pyrazin-2-yl)piperazine-2-carboxamide

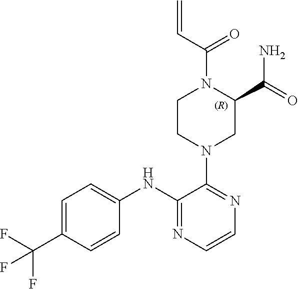

A solution of (2R)-4-(3-{[4-(trifluoromethyl)phenyl]amino}pyrazin-2-yl) piperazine-2-carboxamide (250 mg, 0.68 mmol, 1 equiv) in DCM (5 mL) was treated with TEA (207.17 mg, 2.046 mmol, 3 equiv) for 30 min at 0° C. under nitrogen atmosphere followed by the addition of acryloyl chloride (61.77 mg, 0.68 mmol, 1 equiv) dropwise at 0° C. The resulting mixture was stirred for 1 1 h at room temperature. Desired product could be detected by LCMS. The resulting mixture was extracted with CH₂Cl₂ (2×25 mL). The combined organic layers were washed with H₂O (2×25 mL), dried over anhydrous Na₂SO₄. After filtration, the filtrate was concentrated under reduced pressure. The crude product was purified by reverse phase flash with the following conditions (Acetonitrile: Water/0.05% NaHCO₃ water 33%-70% 18 min) to afford (2R)-1-(prop-2-enoyl)-4-(3-{[4-(trifluoromethyl)phenyl]amino}pyrazin-2-yl) piperazine-2-carboxamide (60 mg 20.91%) as a white solid. LCMS (ES, m/z): [M+H]⁺=421. ¹H NMR (400 MHz, Chloroform-d) δ 9.83 (s, 1H), 8.11 (d, J=8.4 Hz, 2H), 7.95 (d, J=2.8 Hz, 1H), 7.74 (d, J=2.8 Hz, 1H), 7.61 (d, J=8.4 Hz, 2H), 6.72 (d, J=16.8 Hz, 2H), 6.51 (d, J=1.8 Hz, 1H), 5.94 (d, J=10.4 Hz, 1H), 5.57 (s, 1H), 5.35 (s, 1H), 4.28-4.14 (m, 2H), 3.57-3.37 (m, 3H), 2.67 (d, J=12.8 Hz, 1H).

### Example 19: Synthesis of (S)-1-acryloyl-4-(3-((4-(trifluoromethyl)phenyl)amino)pyrazin-2-yl)piperazine-2-carboxamide

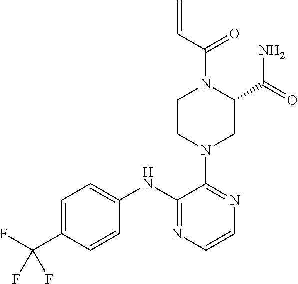 [SMILES: C=CC(=O)N1CCN(c2nccnc2Nc2ccc(C(F)(F)F)cc2)C[C@H]1C(N)=O]

Following the general procedure in scheme 4,1-(tert-butyl) 2-methyl (S)-piperazine-1,2-dicarboxylate was used in step 1. Offered (S)-1-acryloyl-4-(3-((4-(trifluoromethyl)phenyl)amino)pyrazin-2-yl)piperazine-2-carboxamide as white solid. (Positive, ES, m/z): 421.61. ¹H NMR (400 MHz, DMSO) δ 9.60 (d, J=9.1 Hz, 1H), 8.23 (d, J=8.6 Hz, 2H), 8.10-7.89 (m, 2H), 7.86-7.70 (m, 2H), 7.68 (d, J=8.6 Hz, 2H), 6.97-6.79 (m, 1H), 6.21 (dd, J=16.8, 2.3 Hz, 1H), 5.83-5.71 (m, 1H), 5.38-5.00 (m, 1H), 4.38-4.11 (m, 2H), 3.10-2.87 (m, 2H), 2.49-2.30 (m, 2H).

### Example 20: Synthesis of 2-(1-acryloyl-4-(3-((4-(trifluoromethyl)phenyl)amino)pyrazin-2-yl)piperazin-2-yl)acetamide

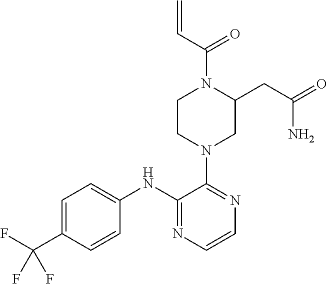 [SMILES: C=CC(=O)N1CCN(c2nccnc2Nc2ccc(C(F)(F)F)cc2)CC1CC(N)=O]

Following the general procedure in scheme 4, tert-butyl 2-(2-methoxy-2-oxoethyl)piperazine-1-carboxylate was used in step 1. Offered 2-(1-acryloyl-4-(3-((4-(trifluoromethyl)phenyl)amino)pyrazin-2-yl)piperazin-2-yl)acetamide as white solid. (Positive, ES, m/z): 435.36. ¹H NMR (400 MHz, DMSO) δ 8.71-8.33 (m, 1H), 7.97-7.80 (m, 4H), 7.66 (d, J=8.4 Hz, 2H), 7.47 (s, 1H), 7.09-6.73 (m, 2H), 6.14 (d, J=16.6 Hz, 1H), 5.72 (d, J=10.4 Hz, 1H), 5.00-4.53 (m, 1H), 4.47-3.87 (m, 1H), 3.75-3.46 (m, 3H), 3.01-2.66 (m, 4H).

### Example 21: Synthesis of (R)-2-(1-acryloyl-4-(3-((4-(trifluoromethyl)phenyl)amino)pyrazin-2-yl)piperazin-2-yl)acetonitrile

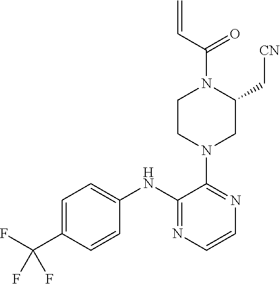 [SMILES: C=CC(=O)N1CCN(c2nccnc2Nc2ccc(C(F)(F)F)cc2)C[C@H]1CC#N]

Following the general procedure in scheme 3, tert-butyl (R)-2-(cyanomethyl)piperazine-1-carboxylate was used in step 2. Offered (R)-2-(1-acryloyl-4-(3-((4-(trifluoromethyl)phenyl)amino)pyrazin-2-yl)piperazin-2-yl)acetonitrile as white solid. (Positive, ES, m/z): 417.26. ¹H NMR (400 MHz, DMSO) δ 8.41 (s, 1H), 8.01-7.79 (m, 4H), 7.69 (d, J=8.5 Hz, 2H), 6.25-6.13 (m, 1H), 5.78 (d, J=10.4 Hz, 1H), 5.10 (s, 1H), 3.84-3.49 (m, 4H), 3.01 (s, 3H).

### Example 22: Synthesis of (S)-2-(1-acryloyl-4-(3-((4-(trifluoromethyl)phen 1 amino)pyrazin-2-yl)piperazin-2-yl)acetonitrile

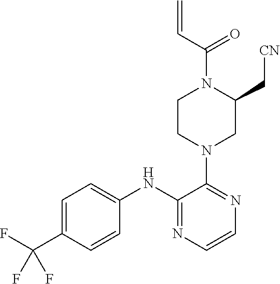 [SMILES: C=CC(=O)N1CCN(c2nccnc2Nc2ccc(C(F)(F)F)cc2)C[C@@H]1CC#N]

Following the general procedure in scheme 3, tert-butyl (S)-2-(cyanomethyl)piperazine-1-carboxylate was used in step 2. Offered (S)-2-(1-acryloyl-4-(3-((4-(trifluoromethyl)phenyl)amino)pyrazin-2-yl)piperazin-2-yl)acetonitrile as white solid. (Positive, ES, m/z): 417.26. ¹H NMR (400 MHz, CDCl₃) δ 8.07-8.02 (m, 2H), 7.98 (d, J=2.2 Hz, 1H), 7.56-7.44 (m, 3H), 6.55 (d, J=2.3 Hz, 1H), 6.19 (s, 1H), 4.38 (d, J=12.3 Hz, 1H), 4.26 (dd, J=12.3, 4.3 Hz, 1H), 4.16 (dp, J=4.2, 2.0 Hz, 1H), 4.11-3.99 (m, 2H), 3.42 (s, 3H), 2.28 (ddp, J=5.5, 3.7, 1.9 Hz, 1H), 2.21-2.08 (m, 1H).

### Example 23. Synthesis of 1-(2-(prop-2-yn-1-yl)-⁴-(³-((4-(trifluoromethyl)phenyl)amino)pyrazin-2-yl)piperazin-1-yl)prop-2-en-1-one

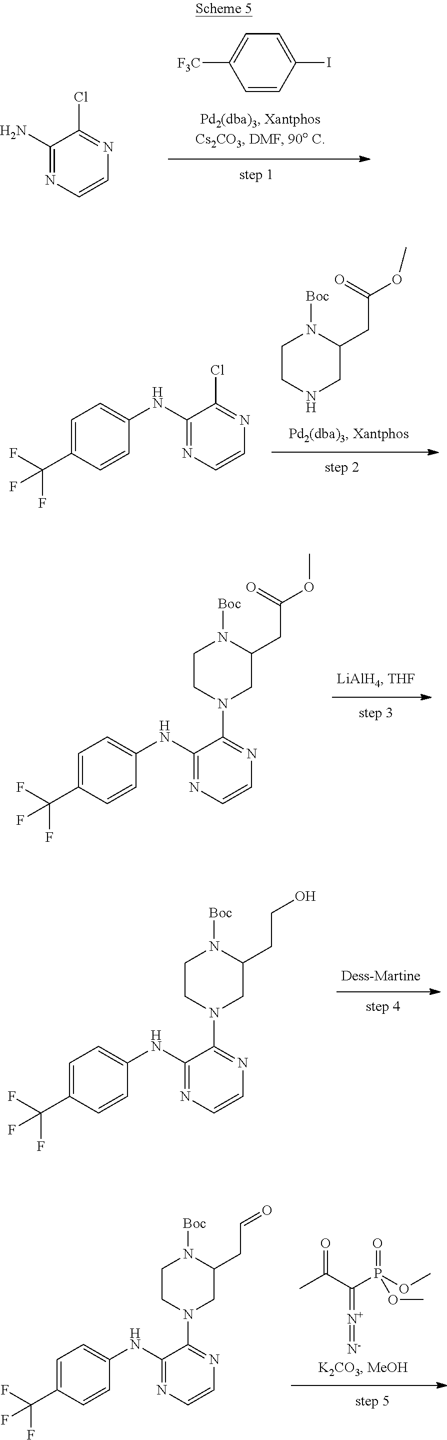

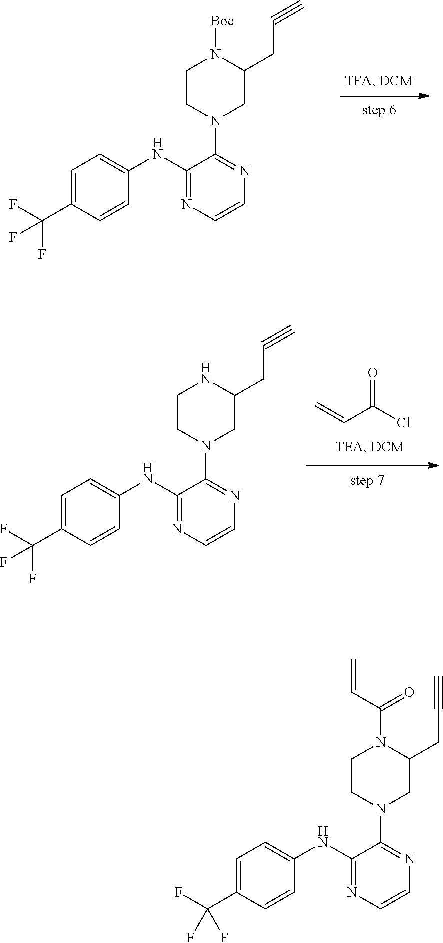

### Step 1: Synthesis of 3-chloro-N-(4-(trifluoromethyl)phenyl)pyrazin-2-amine

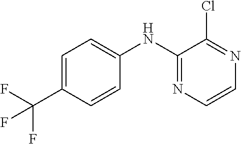 [SMILES: FC(F)(F)c1ccc(Nc2nccnc2Cl)cc1]

Into a 500 mL round-bottom flask were added 3-chloropyrazin-2-amine (20.0 g, 154.38 mmol, 1 equiv)₁-iodo-4-(trifluoromethyl)benzene (46.19 g, 169.82 mmol, 1.1 equiv), Pd₂(dba)₃ (2.0 g, 2.18 mmol, 0.01 equiv), XantPhos (3.0 g, 5.18 mmol, 0.03 equiv), Cs₂CO₃ (150.90 g, 463.14 mmol, 3 equiv) and DMF (200 mL) at room temperature. The resulting mixture was stirred for 6 h at 90° C. under nitrogen atmosphere. The resulting mixture was diluted with water (300 mL). The resulting mixture was extracted with EtOAc (3×300 mL). The combined organic layers were washed with water (2×300 mL), dried over anhydrous Na₂SO₄. After filtration, the filtrate was concentrated under reduced pressure. The residue was purified by silica gel column chromatography, eluted with PE/EA (1:1) to afford 3-chloro-N-[4-(trifluoromethyl)phenyl]pyrazin-2-amine (15 g, 35.51%).

### Step 2: Synthesis of tert-butyl 2-(2-methoxy-2-oxoethyl)-4-(3-{[4-(trifluoromethyl)phenyl]amino}pyrazin-2-yl) piperazine-1-carboxylate

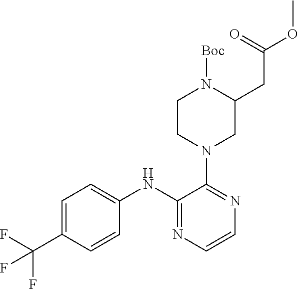 [SMILES: COC(=O)CC1CN(c2nccnc2Nc2ccc(C(F)(F)F)cc2)CCN1C(=O)OC(C)(C)C]

Into a 100 mL round-bottom flask were added 3-chloro-N-[4-(trifluoromethyl)phenyl]pyrazin-2-amine (3.0 g, 10.96 mmol, 1 equiv), tert-butyl 2-(2-methoxy-2-oxoethyl) piperazine-1-carboxylate (3.12 g, 12.06 mmol, 1.1 equiv), Pd₂(dba)₃ (0.3 g, 0.33 mmol, 0.03 equiv), XantPhos (0.45 g, 0.78 mmol, 0.07 equiv), Cs₂CO₃ (10.72 g, 32.89 mmol, 3 equiv) and DMF (30 mL) at room temperature. The resulting mixture was stirred for 4 h at 90° C. under nitrogen atmosphere. The resulting mixture was diluted with water (100 mL). The resulting mixture was extracted with EtOAc (3×100 mL). The combined organic layers were washed with water (2×100 mL), dried over anhydrous Na₂SO₄. After filtration, the filtrate was concentrated under reduced pressure. The residue was purified by silica gel column chromatography, eluted with PE/EA (5:1) to afford tert-butyl 2-(2-methoxy-2-oxoethyl)-4-(3-{[4-(trifluoromethyl)phenyl]amino}pyrazin-2-yl) piperazine-1-carboxylate (2.9 g, 53.38%).

### Step 3: Synthesis of tert-butyl 2-(2-hydroxyethyl)-4-(3-((4-(trifluoromethyl)phenyl)amino)pyrazin-2-yl)piperazine-1-carboxylate

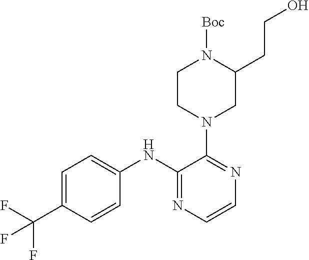 [SMILES: CC(C)(C)OC(=O)N1CCN(c2nccnc2Nc2ccc(C(F)(F)F)cc2)CC1CCO]

A solution of tert-butyl 2-(2-methoxy-2-oxoethyl)-4-(3-{[4-(trifluoromethyl)phenyl]amino}pyrazin-2-yl) piperazine-1-carboxylate (2.2 g, 4.44 mmol, 1 equiv) was treated with THF (30 mL) for 5 min at 0° C. followed by the addition of LiAlH₄ (0.84 g, 22.20 mmol, 5 equiv) at 0° C. The resulting mixture was stirred for 2 h at room temperature. The reaction was quenched with water/ice at 0° C. The resulting mixture was extracted with EtOAc (3×50 mL). The combined organic layers were washed with water (1×50 mL), dried over anhydrous Na₂SO₄. After filtration, the filtrate was concentrated under reduced pressure. The crude product was used in the next step directly without further purification.

### Step 4: Synthesis of tert-butyl 2-(2-oxoethyl)-4-(3-((4-(trifluoromethyl)phenyl)amino)pyrazin-2-yl)piperazine-1-carboxylate

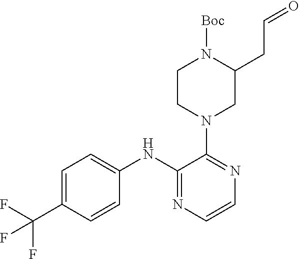 [SMILES: CC(C)(C)OC(=O)N1CCN(c2nccnc2Nc2ccc(C(F)(F)F)cc2)CC1CC=O]

Into a 40 mL vial were added tert-butyl 2-(2-hydroxyethyl)-4-(3-{[4-(trifluoromethyl)phenyl]amino}pyrazin-2-yl) piperazine-1-carboxylate (600 mg, 1.28 mmol, 1 equiv), Dess-Martin (1.09 g, 2.56 mmol, 2 equiv) and DCM (10 mL) at room temperature. The resulting mixture was stirred for 11 h at room temperature. The resulting mixture was diluted with water (20 mL). The resulting mixture was extracted with EtOAc (2×20 mL). The combined organic layers were washed with water (1×20 mL), dried over anhydrous Na₂SO₄. After filtration, the filtrate was concentrated under reduced pressure. The crude product was used in the next step directly without further purification.

### Step 5: Synthesis of tert-butyl 2-(prop-2-yn-1-yl)-⁴-(³-((4-(trifluoromethyl)phenyl)amino)pyrazin-2-yl)piperazine-1-carboxylate

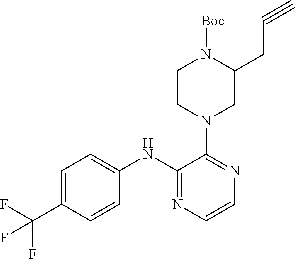 [SMILES: C#CCC1CN(c2nccnc2Nc2ccc(C(F)(F)F)cc2)CCN1C(=O)OC(C)(C)C]

Into a 40 mL vial were added tert-butyl 2-(2-oxoethyl)-4-(3-{[4-(trifluoromethyl)phenyl]amino}pyrazin-2-yl) piperazine-1-carboxylate (158 mg, 0.34 mmol, 1 equiv), seyferth-gilbert homologation (97.81 mg, 0.51 mmol, 1.5 equiv), K₂CO₃ (140.74 mg, 1.02 mmol, 3 equiv) and MeOH (5 mL) at room temperature. The resulting mixture was stirred for 3 h at room temperature. The resulting mixture was diluted with water (10 mL). The resulting mixture was extracted with EtOAc (2×20 mL). The combined organic layers were washed with water (1×10 mL), dried over anhydrous Na₂SO₄. After filtration, the filtrate was concentrated under reduced pressure. The crude product was used in the next step directly without further purification.

### Step 6: Synthesis of 3-(3-(prop-2-yn-1-yl)piperazin-1-yl)-N-(4-(trifluoromethyl)phenyl)pyrazin-2-amine

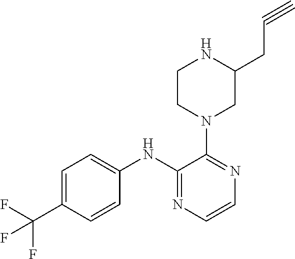 [SMILES: C#CCC1CN(c2nccnc2Nc2ccc(C(F)(F)F)cc2)CCN1]

Into a 40 mL vial were added tert-butyl 2-(prop-2-yn-1-yl)-4-(3-{[4-(trifluoromethyl)phenyl]amino}pyrazin-2-yl) piperazine-1-carboxylate (120 mg, 0.26 mmol, 1 equiv), TFA (6 mL) and DCM (18 mL) at room temperature. The resulting mixture was stirred for 1 h at room temperature. The resulting mixture was concentrated under reduced pressure. The crude product was used in the next step directly without further purification.

### Step 7: Synthesis of 1-(2-(prop-2-yn-1-yl)-4-(3-((4-(trifluoromethyl)phenyl)amino)pyrazin-2-yl)piperazin-1-yl)prop-2-en-1-one

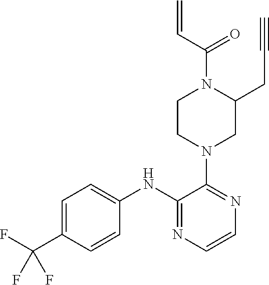 [SMILES: C#CCC1CN(c2nccnc2Nc2ccc(C(F)(F)F)cc2)CCN1C(=O)C=C]

A solution of 3-[3-(prop-2-yn-1-yl) piperazin-1-yl]-N-[4-(trifluoromethyl)phenyl]pyrazin-2-amine (100 mg, 0.27 mmol, 1 equiv) in DCM (10 mL) was treated with TEA (84.01 mg, 0.83 mmol, 3 equiv) for 5 min at 0° C. followed by the addition of acryloyl chloride (25.05 mg, 0.27 mmol, 1 equiv) dropwise at 0° C. The resulting mixture was stirred for 1 h at room temperature. The resulting mixture was diluted with water (10 mL). The resulting mixture was extracted with DCM (2×10 mL). The combined organic layers were washed with water (2×10 mL), dried over anhydrous Na₂SO₄. After filtration, the filtrate was concentrated under reduced pressure. The crude product was purified by reverse phase flash with the following conditions ((Acetonitrile: Water/0.05% ammonia water 25%-80% 19 min)) to afford 1-[2-(prop-2-yn-1-yl)-4-(3-{[4-(trifluoromethyl)phenyl]amino}pyrazin-2-yl) piperazin-1-yl]prop-2-en-1-one (88.3 mg, 75.51%) as a yellow solid. ¹H NMR (400 MHz, Chloroform-d) δ 7.96 (s, 1H), 7.88-7.75 (m, 3H), 7.61 (d, J=8.5 Hz, 2H), 7.31 (m, 1H), 6.64 (d, J=13.9 Hz, 11H), 6.39 (d, J=16.7 Hz, 1H), 5.81 (m, 1H), 4.85 (m, 1H), 4.22 (m, 1H), 3.76 (m, 2H), 3.35 (d, J=12.2 Hz, 1H), 3.09 (m, 2H), 2.88 (m, 1H), 2.70 (m, 1H), 2.02 (s, 1H).

### Example 24: Synthesis of 1-(3-methyl-4-(3-((4-(trifluoromethyl)phenyl)amino)pyrazin-2-yl)piperazin-1-yl)prop-2-en-1-one

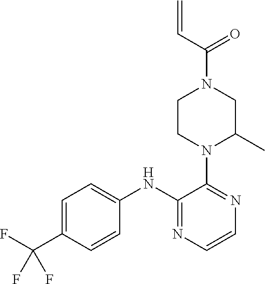 [SMILES: C=CC(=O)N1CCN(c2nccnc2Nc2ccc(C(F)(F)F)cc2)C(C)C1]

Following the general procedure in scheme 3, tert-butyl 3-methylpiperazine-1-carboxylate was used in step 2. Offered 1-(3-methyl-4-(3-((4-(trifluoromethyl)phenyl)amino)pyrazin-2-yl)piperazin-1-yl)prop-2-en-1-one as white solid. (Positive, ES, m/z): 392.26. ¹H NMR (400 MHz, DMSO) δ 8.69-8.61 (m, 1H), 7.97 (d, J=9.4 Hz, 3H), 7.89 (d, J=2.8 Hz, 1H), 7.66 (d, J=8.5 Hz, 2H), 6.87 (dd, J=16.7, 10.4 Hz, 1H), 6.17 (d, J=17.1 Hz, 1H), 5.73 (dd, J=10.4, 2.4 Hz, 1H), 4.06-3.68 (m, 5H), 3.22 (d, J=9.6 Hz, 2H), 2.86 (ddd, J=11.8, 6.9, 3.4 Hz, 2H), 0.92 (d, J=6.0 Hz, 3H).

### Example 25. Synthesis of 1-[4-(3-{[4-(trifluoromethyl)phenyl]amino}pyrazin-2-yl)-3,6-dihydro-2H-pyridin-1-yl]prop-2-en-1-one

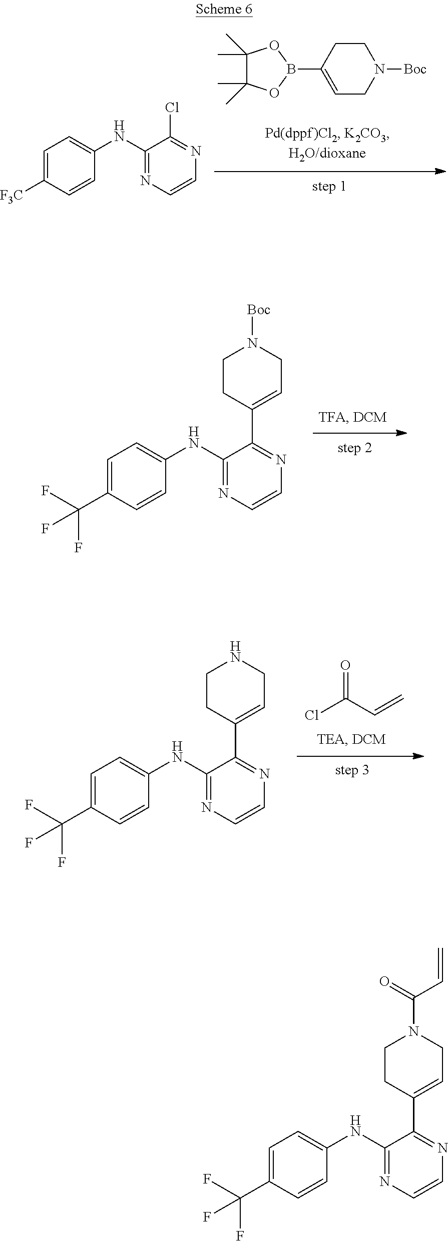

### Step 1: Synthesis of tert-butyl 4-(3-{[4-(trifluoromethyl)phenyl]amino}pyrazin-2-yl)-3,6-dihydro-2H-pyridine-1-carboxylate

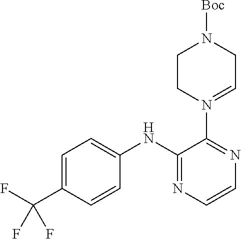

A solution of 3-chloro-N-[4-(trifluoromethyl)phenyl]pyrazin-2-amine (1.00 g, 3.65 mmol, 1.00 equiv), tert-butyl 4-(4,4,5,5-tetramethyl-1,3,2-dioxaborolan-2-yl)-3,6-dihydro-2H-pyridine-1-carboxylate (1.24 g, 4.02 mmol, 1.10 equiv), K₂CO₃ (1.01 g, 7.31 mmol, 2.00 equiv) and Pd(dppf)Cl₂ (0.27 g, 0.37 mmol, 0.10 equiv) in H₂O (3.00 mL) and dioxane (9.00 mL) was stirred for 3 h at 90° C. under N₂ atmosphere. The mixture was cooled and extracted with EA (3×30.00 mL). The combined organic layers were washed with H₂O (3×30.00 mL), dried over anhydrous Na₂SO₄. After filtration, the filtrate was concentrated under reduced pressure. The residue was purified by silica gel column chromatography, eluted with PE:EA (3:1) to afford tert-butyl 4-(3-{[4-(trifluoromethyl)phenyl]amino}pyrazin-2-yl)-3,6-dihydro-2H-pyridine-1-carboxylate (1.00 g, 65.08%) as a yellow solid. LCMS-PH-CAL-Y-V2454220-1:(ES, m/z): [M+H]⁺=421

### Step 2: Synthesis of 3-(1,2,3,6-tetrahydropyridin-4-yl)-N-[4-(trifluoromethyl)phenyl]pyrazin-2-amine (TFA salt)

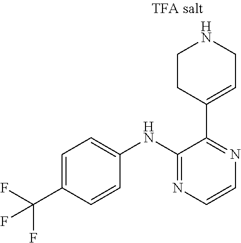 [SMILES: FC(F)(F)c1ccc(Nc2nccnc2C2=CCNCC2)cc1]

Into a 40 mL vessel were placed a solution of tert-butyl 4-(3-{[4-(trifluoromethyl)phenyl]amino}pyrazin-2-yl)-3,6-dihydro-2H-pyridine-1-carboxylate (370.00 mg, 0.88 mmol, 1.00 equiv) in DCM (1.50 mL) and TFA (1.50 mL). The mixture was stirred for 1 h at room temperature before concentrating under vacuum. This resulted in 3-(1,2,3,6-tetrahydropyridin-4-yl)-N-[4-(trifluoromethyl)phenyl]pyrazin-2-amine (230 mg, 81.59%) as a yellow oil. LCMS:(ES, m/z): [M+H]⁺=321.

### Step 3: Synthesis of 1-[4-(3-{1[4-(trifluoromethyl)phenyl]amino}pyrazin-2-yl)-3,6-dihydro-2H-pyridin-1-yl]prop-2-en-1-one

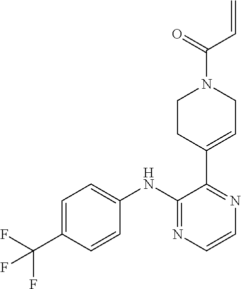 [SMILES: C=CC(=O)N1CC=C(c2nccnc2Nc2ccc(C(F)(F)F)cc2)CC1]

Into a 40 mL vessel were placed a solution of 3-(1,2,3,6-tetrahydropyridin-4-yl)-N-[4-(trifluoromethyl)phenyl]pyrazin-2-amine (210.00 mg, 0.66 mmol, 1.00 equiv) and TEA (199.03 mg, 1.97 mmol, 3.00 equiv) in DCM (3.00 mL). Acryloyl chloride (59.34 mg, 0.66 mmol, 1.00 equiv) was then added at 0° C. The resulted mixture was stirred for 1 h at room temperature. After the reaction was completed, the mixture was diluted with EA (45.00 mL), washed with H₂O (3×15.00 mL), dried over anhydrous Na₂SO₄. After filtration, the filtrate was concentrated under reduced pressure. The crude product (300.00 mg) was purified by Prep-HPLC with the following conditions (Column: Welch Xtimate C18 ExRS, 250 mm, 10 m; Mobile Phase A: Water (0.05% NH₃·H₂O), Mobile Phase B: ACN; Flow rate: 60 mL/min; Gradient: 90% B to 90% B in 10 min, 90% B; Wave Length: 254 nm; RT1(min): 7; Number Of Runs: 5) to afford 1-[4-(3-{[4-(trifluoromethyl)phenyl]amino}pyrazin-2-yl)-3,6-dihydro-2H-pyridin-1-yl]prop-2-en-1-one (70.00 mg, 28.52%) as a yellow solid. LCMS:(ES, m/z): [M+H]⁺=375. ¹H NMR (400 MHz, DMSO-d₆): δ 8.74 (s, 1H), 8.11 (s, 2H), 7.81 (d, J=11.6 Hz, 2H), 7.61 (d, J=12.0 Hz, 2H), 6.95-6.86 (m, 1H), 6.33-6.13 (m, 2H), 5.74-5.70 (m, 1H), 4.29-4.22 (m, 2H), 3.80-3.78 (m, 2H), 2.61-2.58 (m, 2H).

### Example 26. Synthesis of 1-[4-(3-{[4-(trifluoromethyl)phenyl]amino}pyrazin-2-yl)piperidin-1-yl]prop-2-en-1-one

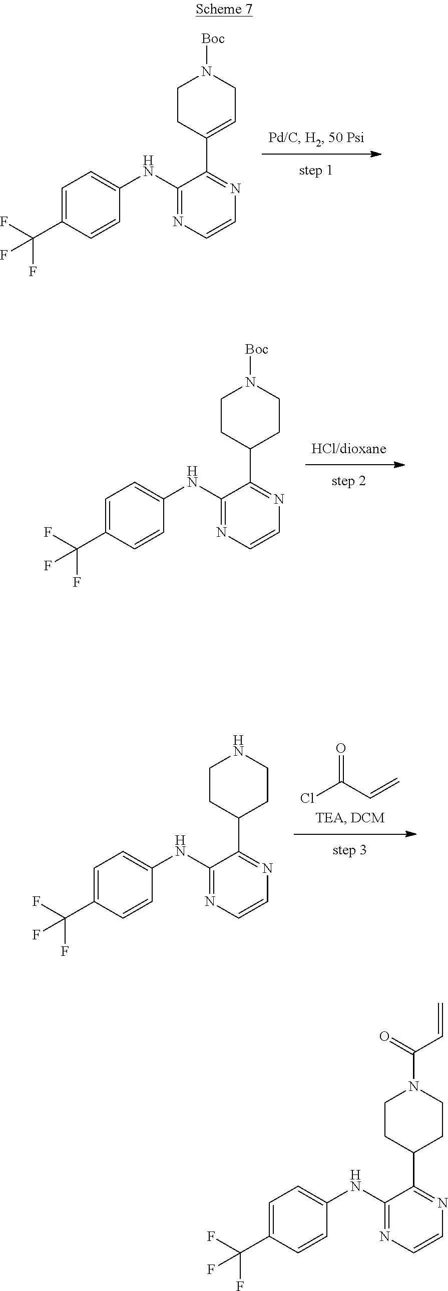

### Step 1: Synthesis of tert-butyl 4-(3-{[4-(trifluoromethyl)phenyl]amino}pyrazin-2-yl)piperidine-1-carboxylate

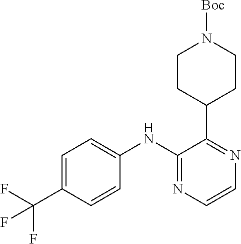 [SMILES: CC(C)(C)OC(=O)N1CCC(c2nccnc2Nc2ccc(C(F)(F)F)cc2)CC1]

A solution of tert-butyl 4-(3-{[4-(trifluoromethyl)phenyl]amino}pyrazin-2-yl)-3,6-dihydro-2H-pyridine-1-carboxylate (500.00 mg, 1.189 mmol, 1.00 equiv) and Pd/C (12.66 mg, 0.12 mmol, 0.10 equiv) in MeOH (5.00 mL) was stirred overnight at 50° C. under H₂ (50 Psi) atmosphere. The resulting mixture was filtered and the filter cake was washed with MeOH (3×5.00 mL). The filtrate was concentrated under reduced pressure. This resulted in tert-butyl 4-(3-{[4-(trifluoromethyl)phenyl]amino}pyrazin-2-yl)piperidine-1-carboxylate (500.00 mg, 99.52%) as a yellow solid. LCMS ES, m/z: [M+H]⁺=423.

### Step 2: Synthesis of 3-(piperidin-4-yl)-N-[4-(trifluoromethyl)phenyl]pyrazin-2-amine

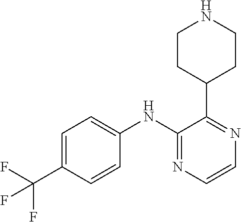 [SMILES: FC(F)(F)c1ccc(Nc2nccnc2C2CCNCC2)cc1]

A solution of tert-butyl 4-(3-{[4-(trifluoromethyl)phenyl]amino}pyrazin-2-yl)piperidine-1-carboxylate (500.00 mg, 1.18 mmol, 1.00 equiv) and HCl/dioxane solution (6.00 mL) in DCM (2.00 mL) was stirred for 1 h at 0° C. to room temperature. The resulting mixture was concentrated under vacuum. This resulted in 3-(piperidin-4-yl)-N-[4-(trifluoromethyl)phenyl]pyrazin-2-amine (280.00 mg, 73.39%) as a yellow solid. LCMS ES, m/z: [M+H]⁺=323.

### Step 3: Synthesis of 1-[4-(3-{[4-(trifluoromethyl)phenyl]amino}pyrazin-2-yl)piperidin-1-yl]prop-2-en-1-one

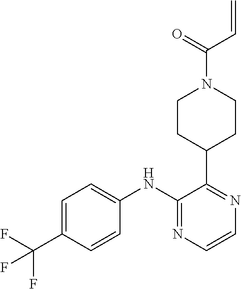 [SMILES: C=CC(=O)N1CCC(c2nccnc2Nc2ccc(C(F)(F)F)cc2)CC1]

A solution of 3-(piperidin-4-yl)-N-[4-(trifluoromethyl)phenyl]pyrazin-2-amine (260.00 mg, 0.81 mmol, 1.00 equiv) and TEA (244.87 mg, 2.42 mmol, 3.00 equiv) in DCM (3.00 mL) was stirred at 0° C. before acryloyl chloride (73.01 mg, 0.81 mmol, 1.00 equiv) was added. The resulting mixture was stirred for 1 h at room temperature. The resulting mixture was extracted with EA (3×10.00 mL). The combined organic layers were washed with H₂O (3×10.00 mL), dried over anhydrous Na₂SO₄. After filtration, the filtrate was concentrated under reduced pressure. The crude product was purified by (Column: Welch Xtimate C18 ExRS, 250 mm, 10 μm; Mobile Phase A: Water (0.05% NH₃·H₂O), Mobile Phase B: ACN; Flow rate: 60 mL/min; Gradient: 33% B to 56% B in 16 min, 56% B; Wave Length: 254 nm; RT1(min): 7; Number Of Runs: 5) to afford 1-[4-(3-{[4-(trifluoromethyl)phenyl]amino}pyrazin-2-yl)piperidin-1-yl]prop-2-en-1-one (45.00 mg, 14.82%) as a white solid. LCMS ES, m/z: 377. ¹H NMR (400 MHz, DMSO-d₆): δ 8.80 (s, 1H), 8.06 (s, 2H), 7.84 (d, J=4.2 Hz, 2H), 7.64 (d, J=4.4 Hz, 2H), 6.90-6.81 (m, 1H), 6.15-6.10 (m, 1H), 5.70-5.66 (m, 1H), 4.71-4.52 (m, 1H), 4.38-4.14 (m, 1H), 3.62-3.44 (m, 1H), 3.32-3.30 (m, 1H), 2.72-2.52 (m, 1H), 1.89-1.85 (m, 2H), 1.72-1.58 (m, 2H).

### Example 27: Synthesis of 1-(4-(3-((4-(trifluoromethyl)phenyl)amino)pyrazin-2-yl)-1,4-diazepan-1-yl)prop-2-en-1-one

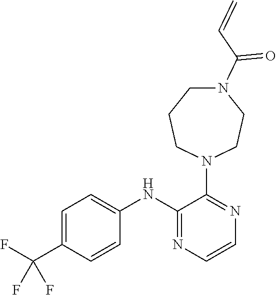 [SMILES: C=CC(=O)N1CCCN(c2nccnc2Nc2ccc(C(F)(F)F)cc2)CC1]

Following the general procedure in scheme 3, tert-butyl 1,4-diazepane-1-carboxylate was used in step 2. Offered 1-(4-(3-((4-(trifluoromethyl)phenyl)amino)pyrazin-2-yl)-1,4-diazepan-1-yl)prop-2-en-1-one as white solid. (Positive, ES, m/z): 392.26. ¹H NMR (400 MHz, DMSO) δ 8.64-8.44 (m, 1H), 7.86-7.68 (m, 4H), 7.60 (dd, J=8.6, 6.1 Hz, 2H), 6.85-6.62 (m, 1H), 6.22-5.99 (m, 1H), 5.75-5.58 (m, 1H), 3.85-3.62 (m, 3H), 3.59-3.37 (m, 5H), 1.90-1.74 (m, 2H).

### Example 28. Synthesis of 1-(4-(3-(4-(trifluoromethyl)phenyl)pyrazin-2-yl)piperazin-1-yl)prop-2-en-1-one

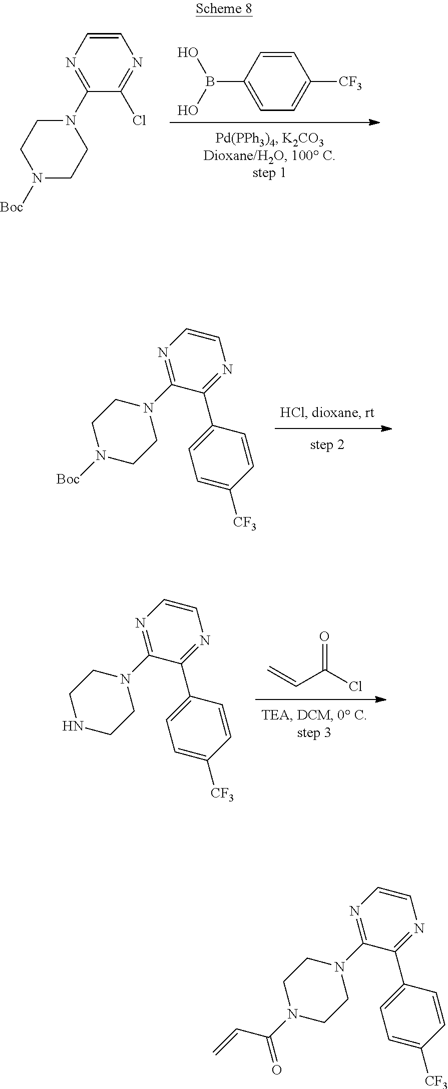

### Step 1: Synthesis of tert-butyl 4-(3-(4-(trifluoromethyl)phenyl)pyrazin-2-yl)piperazine-1-carboxylate

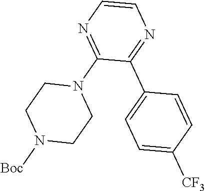 [SMILES: CC(C)(C)OC(=O)N1CCN(c2nccnc2-c2ccc(C(F)(F)F)cc2)CC1]

To a stirred mixture of tert-butyl 4-(3-chloropyrazin-2-yl)piperazine-1-carboxylate (0.20 g, 0.67 mmol, 1.00 equiv) and (4-(trifluoromethyl)phenyl)boronic acid (0.153 g, 0.80 mmol, 1.20 equiv), and K₂CO₃ (0.32 g, 2.34 mmol, 3.5 equiv) in dioxane (10.00 mL) were added Pd(PPh₃)₄(0.077 g, 0.067 mmol, 0.1 equiv) in portions at room temperature under nitrogen atmosphere. The resulting mixture was stirred overnight at 100° C. under nitrogen atmosphere. The mixture was allowed to cool down to room temperature and extracted with NaCl (sat. aq. slon.) and EA. The organic layer dried over Na₂SO₄, concentrated in vacuo. Purified by silica gel column chromatography, eluted with DCM:EA (10:1) to afford tert-butyl 4-(3-(4-(trifluoromethyl)phenyl)pyrazin-2-yl)piperazine-1-carboxylate (178.00 mg, 65.11% yield) as yellow solid.

### Step 2: Synthesis of 2-(piperazin-1-yl)-3-(4-(trifluoromethyl)phenyl)pyrazine

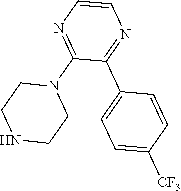 [SMILES: FC(F)(F)c1ccc(-c2nccnc2N2CCNCC2)cc1]

To a stirred mixture of tert-butyl 4-(3-(4-(trifluoromethyl)phenyl)pyrazin-2-yl)piperazine-1-carboxylate (178 mg, 0.44 mmol, 1.00 equiv) in Dioxane (2.00 mL) were added HCl(gas) in 1,4-dioxane (2.18 mL, 8.72 mmol, 20.00 equiv) at 0° C. The resulting mixture was stirred for 2 h at room temperature. The resulting mixture was concentrated under reduced pressure to afford 2-(piperazin-1-yl)-3-(4-(trifluoromethyl)phenyl)pyrazine (130 mg, 96.75% yield) as a yellow oil.

### Step 3: Synthesis of 1-(4-(3-(4-(trifluoromethyl)phenyl)pyrazin-2-yl)piperazin-1-yl)prop-2-en-1-one

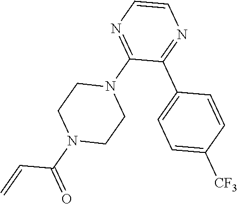 [SMILES: C=CC(=O)N1CCN(c2nccnc2-c2ccc(C(F)(F)F)cc2)CC1]

2-(piperazin-1-yl)-³-(4-(trifluoromethyl)phenyl)pyrazine (100 mg, 0.32 mmol, 1.00 equiv) and TEA (134 mmL, 0.81 mmol, 2.50 equiv) in DCM (2.00 mL) at 0° C. To the above mixture was added acryloyl chloride (31 mmL, 0.39 mmol, 1.20 equiv). The resulting mixture was stirred for additional 0.5 h at 0° C. The reaction was monitored by LCMS. The resulting mixture was added 50.00 mL H₂O and extracted with ethyl acetate (50.00 mL×3). The combined organic layers were washed with brine (100.00 mL), dried over anhydrous Na₂SO₄. After filtration, the filtrate was concentrated under reduced pressure. The crude product was purified by silica gel column chromatography, eluted with DCM:EA (3:7) to afford 1-(4-(3-(4-(trifluoromethyl)phenyl)pyrazin-2-yl)piperazin-1-yl)prop-2-en-1-one (65 mg, 55.3% yield) as white solid. (Positive, ES, m/z): 363.26. ¹H NMR (400 MHz, DMSO) δ 8.31-8.21 (m, 2H), 8.14 (d, J=8.1 Hz, 2H), 7.85 (d, J=8.2 Hz, 2H), 6.77 (dd, J=16.6, 10.4 Hz, 1H), 6.10 (dd, J=16.7, 2.4 Hz, 1H), 5.66 (dd, J=10.4, 2.4 Hz, 1H), 3.58 (s, 4H), 3.12 (d, J=15.6 Hz, 4H).

### Example 29: Synthesis of 1-(4-(3-(3-(trifluoromethyl)phenyl)pyrazin-2-yl)piperazin-1-yl)prop-2-en-1-one

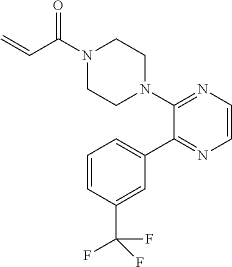 [SMILES: C=CC(=O)N1CCN(c2nccnc2-c2cccc(C(F)(F)F)c2)CC1]

Following the general procedure in scheme 8, (3-(trifluoromethyl)phenyl)boronic acid was used in step 1. Offered 1-(4-(3-(3-(trifluoromethyl)phenyl)pyrazin-2-yl)piperazin-1-yl)prop-2-en-1-one as white solid. (Positive, ES, m/z): 363.26. ¹H NMR (400 MHz, DMSO) δ 8.29 (d, J=2.5 Hz, 1H), 8.27-8.23 (m, 3H), 7.87-7.70 (m, 2H), 6.77 (dd, J=16.6, 10.4 Hz, 1H), 6.10 (dd, J=16.7, 2.4 Hz, 1H), 5.67 (dd, J=10.4, 2.4 Hz, 1H), 3.56 (d, =12.8 Hz, 4H), 3.10 (d, J=6.5 Hz, 4H).

### Example 30: Synthesis of 1-(4-(3-((3-(trifluoromethyl)phenyl)amino)pyrazin-2-yl)piperazin-1-yl)prop-2-en-1-one

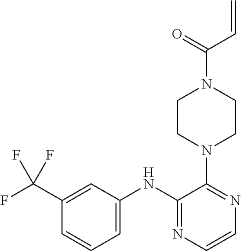 [SMILES: C=CC(=O)N1CCN(c2nccnc2Nc2cccc(C(F)(F)F)c2)CC1]

Following the general procedure in scheme 1,3-(trifluoromethyl)aniline was used in step 2. Offered 1-(4-(3-((3-(trifluoromethyl)phenyl)amino)pyrazin-2-yl)piperazin-1-yl)prop-2-en-1-one as white solid. (Positive, ES, m/z): 378.58. ¹H NMR (400 MHz, DMSO) δ 8.54 (s, 1H), 8.11 (t, J=2.0 Hz, 1H), 8.02 (d, J=8.2 Hz, 1H), 7.90-7.75 (m, 2H), 7.52 (t, J=8.0 Hz, 1H), 7.33-7.25 (m, 1H), 6.85 (dd, J=16.7, 10.5 Hz, 1H), 6.14 (dd, J=16.7, 2.4 Hz, 1H), 5.71 (dd, J=10.4, 2.4 Hz, 1H), 3.78 (d, J=13.6 Hz, 4H), 3.13 (s, 4H).

### Example 31: Synthesis of 1-(4-(3-((4-(pentafluoro-16-sulfaneyl)phenyl)amino)pyrazin-2-yl)piperazin-1-yl)prop-2-en-1-one

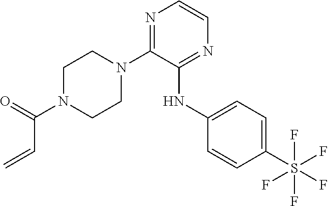 [SMILES: C=CC(=O)N1CCN(c2nccnc2Nc2ccc(S(F)(F)(F)(F)F)cc2)CC1]

Following the general procedure in scheme 1,4-(pentafluoro-16-sulfaneyl)aniline was used in step 2. Offered 1-(4-(3-((4-(pentafluoro-16-sulfaneyl)phenyl)amino)pyrazin-2-yl)piperazin-1-yl)prop-2-en-1-one as white solid. (Positive, ES, m/z): 436.1. ¹H NMR (400 MHz, DMSO) δ 8.72 (s, 1H), 7.92-7.83 (m, 4H), 7.83-7.74 (m, 2H), 6.84 (dd, J=16.7, 10.5 Hz, 1H), 6.14 (dd, J=16.7, 2.4 Hz, 1H), 5.71 (dd, J=10.4, 2.4 Hz, 1H), 3.77 (d, J=12.5 Hz, 4H), 3.15 (d, J=6.1 Hz, 4H).

### Example 32: Synthesis of 1-(4-(3-((4-chlorophenyl)amino)pyrazin-2-yl)piperazin-1-yl)prop-2-en-1-one

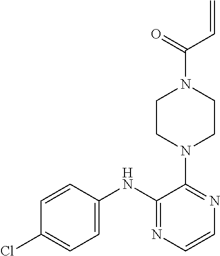 [SMILES: C=CC(=O)N1CCN(c2nccnc2Nc2ccc(Cl)cc2)CC1]

Following the general procedure in scheme 1, 4-chloroaniline was used in step 2. Offered 1-(4-(3-((4-chlorophenyl)amino)pyrazin-2-yl)piperazin-1-yl)prop-2-en-1-one as white solid. (Positive, ES, m/z): 344.1. ¹H NMR (400 MHz, DMSO) δ 8.31 (s, 1H), 7.81 (d, J=2.8 Hz, 1H), 7.78-7.66 (m, 3H), 7.38-7.23 (m, 2H), 6.84 (dd, J=16.7, 10.5 Hz, 1H), 6.14 (dd, J=16.7, 2.4 Hz, 1H), 5.71 (dd, J=10.4, 2.4 Hz, 1H), 3.77 (d, J=9.7 Hz, 4H), 3.10 (t, J=4.6 Hz, 4H).

### Example 33: Synthesis of 1-(4-(3-((4-fluorophenyl)amino)pyrazin-2-yl)piperazin-1-yl)prop-2-en-1-one

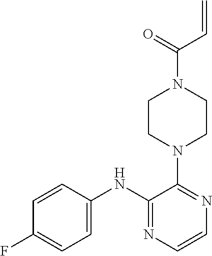 [SMILES: C=CC(=O)N1CCN(c2nccnc2Nc2ccc(F)cc2)CC1]

Following the general procedure in scheme 1, 4-fluoroaniline was used in step 2. Offered 1-(4-(3-((4-fluorophenyl)amino)pyrazin-2-yl)piperazin-1-yl)prop-2-en-1-one as white solid. (Positive, ES, m/z): 328.46. ¹H NMR (400 MHz, DMSO) δ 8.20 (s, 1H), 7.77 (d, J=2.8 Hz, 1H), 7.74-7.63 (m, 3H), 7.14 (t, J=8.9 Hz, 2H), 6.85 (dd, J=16.7, 10.4 Hz, 1H), 6.14 (dd, J=16.7, 2.4 Hz, 1H), 5.71 (dd, J=10.4, 2.4 Hz, 1H), 3.78 (d, J=11.7 Hz, 4H), 3.17-2.95 (m, 4H).

### Example 34: Synthesis of 1-(4-(3-((4-(trifluoromethoxy)phenyl)amino)pyrazin-2-yl)piperazin-1-yl)prop-2-en-1-one

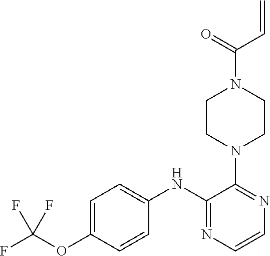 [SMILES: C=CC(=O)N1CCN(c2nccnc2Nc2ccc(OC(F)(F)F)cc2)CC1]

Following the general procedure in scheme 1, 4-(trifluoromethoxy)aniline was used in step 2. Offered 1-(4-(3-((4-(trifluoromethoxy)phenyl)amino)pyrazin-2-yl)piperazin-1-yl)prop-2-en-1-one as white solid. (Positive, ES, m/z): 394.55. ¹H NMR (400 MHz, DMSO) δ 8.39 (s, 1H), 7.87-7.69 (m, 4H), 7.35-7.26 (m, 2H), 6.86 (dd, J=16.7, 10.4 Hz, 1H), 6.16 (dd, J=16.7, 2.4 Hz, 1H), 5.72 (dd, J=10.4, 2.4 Hz, 1H), 3.80 (s, 4H), 3.13 (s, 4H).

### Example 35: Synthesis of 1-(4-(3-((4-(difluoromethoxy)phenyl)amino)pyrazin-2-yl)piperazin-1-yl)prop-2-en-1-one

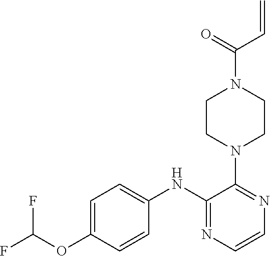 [SMILES: C=CC(=O)N1CCN(c2nccnc2Nc2ccc(OC(F)F)cc2)CC1]

Following the general procedure in scheme 1, 4-(difluoromethoxy)aniline was used in step 2. Offered 1-(4-(3-((4-(difluoromethoxy)phenyl)amino)pyrazin-2-yl)piperazin-1-yl)prop-2-en-1-one as white solid. (Positive, ES, m/z): 376.1. ¹H NMR (400 MHz, DMSO) δ 8.26 (s, 1H), 7.79 (d, J=2.8 Hz, 1H), 7.72 (dd, J=9.4, 2.5 Hz, 3H), 7.36-6.94 (m, 3H), 6.89-6.76 (m, 1H), 6.16 (dd, J=16.7, 2.4 Hz, 1H), 5.72 (dd, J=10.5, 2.4 Hz, 1H), 3.87-3.70 (m, 4H), 3.12 (p, J=4.0 Hz, 4H).

### Example 36: Synthesis of 1-(4-(3-((4-(fluoromethoxy)phenyl)amino)pyrazin-2-yl)piperazin-1-yl)prop-2-en-1-one

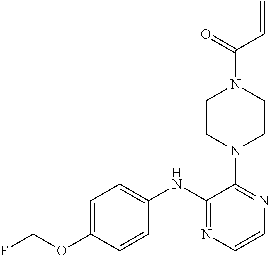 [SMILES: C=CC(=O)N1CCN(c2nccnc2Nc2ccc(OCF)cc2)CC1]

Following the general procedure in scheme 1, 4-(fluoromethoxy)aniline was used in step 2. Offered 1-(4-(3-((4-(fluoromethoxy)phenyl)amino)pyrazin-2-yl)piperazin-1-yl)prop-2-en-1-one as white solid. (Positive, ES, m/z): 358.3. ¹H NMR (400 MHz, DMSO) δ 8.15 (s, 1H), 7.77 (d, J=2.8 Hz, 1H), 7.70 (d, J=2.9 Hz, 1H), 7.68-7.58 (m, 2H), 7.14-7.02 (m, 2H), 6.86 (dd, J=16.7, 10.4 Hz, 1H), 6.16 (dd, J=16.7, 2.4 Hz, 1H), 5.89 (s, 1H), 5.83-5.65 (m, 2H), 3.79 (d, J=12.2 Hz, 4H), 3.11 (d, J=6.5 Hz, 4H).

### Example 37: Synthesis of 1-(4-(3-((3-methoxy-4-(trifluoromethyl)phenyl)amino)pyrazin-2-yl)piperazin-1-yl)prop-2-en-1-one

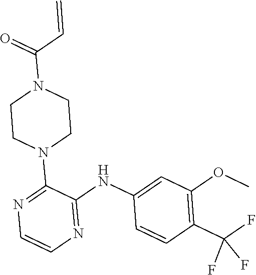 [SMILES: C=CC(=O)N1CCN(c2nccnc2Nc2ccc(C(F)(F)F)c(OC)c2)CC1]

Following the general procedure in scheme 1, 3-methoxy-4-(trifluoromethyl)aniline was used in step 2. Offered 1-(4-(3-((3-methoxy-4-(trifluoromethyl)phenyl)amino)pyrazin-2-yl)piperazin-1-yl)prop-2-en-1-one as white solid. (Positive, ES, m/z): 408.34. ¹H NMR (400 MHz, DMSO) δ 8.58 (s, 1H), 7.94-7.82 (m, 2H), 7.65 (s, 1H), 7.55-7.46 (m, 2H), 6.86 (dd, J=16.7, 10.4 Hz, 1H), 6.16 (dd, J=16.6, 2.4 Hz, 1H), 5.73 (dd, J=10.3, 2.4 Hz, 1H), 3.97-3.67 (m, 7H), 3.16 (d, J=5.9 Hz, 4H).

### Example 38: Synthesis of 4-((3-(4-acryloylpiperazin-1-yl)pyrazin-2-yl)amino)benzonitrile

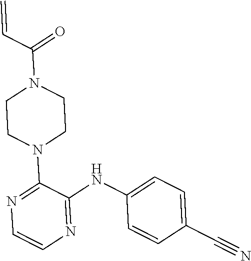 [SMILES: C=CC(=O)N1CCN(c2nccnc2Nc2ccc(C#N)cc2)CC1]

Following the general procedure in scheme 1, 4-aminobenzonitrile was used in step 2. Offered 4-((3-(4-acryloylpiperazin-1-yl)pyrazin-2-yl)amino)benzonitrile as white solid. (Positive, ES, m/z): 335.24. ¹H NMR (400 MHz, DMSO) δ 8.78 (s, 1H), 8.02-7.85 (m, 4H), 7.82-7.66 (m, 2H), 6.92-6.76 (m, 1H), 6.16 (dt, J=16.7, 1.9 Hz, 1H), 5.72 (dt, J=10.6, 1.8 Hz, 1H), 3.78 (d, J=12.8 Hz, 4H), 3.16 (s, 4H).

### Example 39: Synthesis of 1-(4-(3-((5-(trifluoromethyl)pyridin-2-yl)amino)pyrazin-2-yl)piperazin-1-yl)prop-2-en-1-one

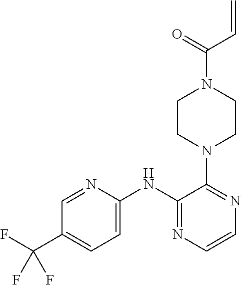 [SMILES: C=CC(=O)N1CCN(c2nccnc2Nc2ccc(C(F)(F)F)cn2)CC1]

Following the general procedure in scheme 1, 5-(trifluoromethyl)pyridin-2-amine was used in step 2. Offered 1-(4-(3-((5-(trifluoromethyl)pyridin-2-yl)amino)pyrazin-2-yl)piperazin-1-yl)prop-2-en-1-one as white solid. (Positive, ES, m/z): 379.27. ¹H NMR (400 MHz, DMSO) δ 9.01 (s, 1H), 8.63 (dt, J=2.1, 1.0 Hz, 1H), 8.15-8.04 (m, 2H), 7.99 (s, 2H), 6.85 (dd, J=16.7, 10.4 Hz, 1H), 6.15 (dd, J=16.7, 2.4 Hz, 1H), 5.72 (dd, J=10.4, 2.4 Hz, 1H), 3.74 (d, J=14.9 Hz, 4H), 3.16 (s, 4H).

### Example 40: Synthesis of 1-(4-(3-((5-(difluoromethyl)pyridin-2-yl)amino)pyrazin-2-yl)piperazin-1-yl)prop-2-en-1-one

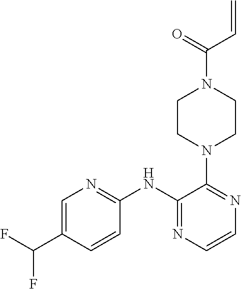 [SMILES: C=CC(=O)N1CCN(c2nccnc2Nc2ccc(C(F)F)cn2)CC1]

Following the general procedure in scheme 1, 5-(difluoromethyl)pyridin-2-amine was used in step 2. Offered 1-(4-(3-((5-(difluoromethyl)pyridin-2-yl)amino)pyrazin-2-yl)piperazin-1-yl)prop-2-en-1-one as white solid. (Positive, ES, m/z): 361.2. ¹H NMR (400 MHz, DMSO) δ 8.74 (s, 1H), 8.48 (s, 1H), 8.11 (d, J=8.7 Hz, 1H), 8.06-7.92 (m, 3H), 7.25-6.90 (m, 1H), 6.85 (dd, J=16.7, 10.4 Hz, 1H), 6.15 (dd, J=16.7, 2.4 Hz, 1H), 5.72 (dd, J=10.4, 2.4 Hz, 1H), 3.75 (d, J=15.6 Hz, 4H), 3.14 (s, 4H).

### Example 41: Synthesis of 1-(4-(3-((5-(trifluoromethyl)pyrimidin-2-yl)amino)pyrazin-2-yl)piperazin-1-yl)prop-2-en-1-one

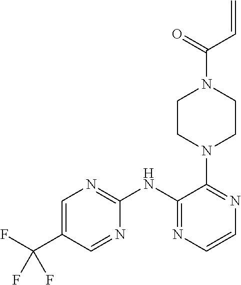 [SMILES: C=CC(=O)N1CCN(c2nccnc2Nc2ncc(C(F)(F)F)cn2)CC1]

Following the general procedure in scheme 1, 5-(trifluoromethyl)pyrimidin-2-amine was used in step 2. Offered 1-(4-(3-((5-(trifluoromethyl)pyrimidin-2-yl)amino)pyrazin-2-yl)piperazin-1-yl)prop-2-en-1-one as white solid. (Positive, ES, m/z): 380.22. ¹H NMR (400 MHz, DMSO) δ 10.36 (s, 1H), 8.89-8.74 (m, 2H), 8.15 (d, J=2.6 Hz, 1H), 7.99 (d, J=2.6 Hz, 1H), 6.79 (dd, J=16.7, 10.5 Hz, 1H), 6.11 (dd, J=16.6, 2.4 Hz, 1H), 5.69 (dd, J=10.4, 2.4 Hz, 1H), 3.50 (s, 4H), 3.32-3.28 (m, 4H).

### Example 42. Synthesis of 1-(4-(3-(methyl(5-(trifluoromethyl)pyridin-2-yl)amino)pyrazin-2-yl)piperazin-1-yl)prop-2-en-1-one

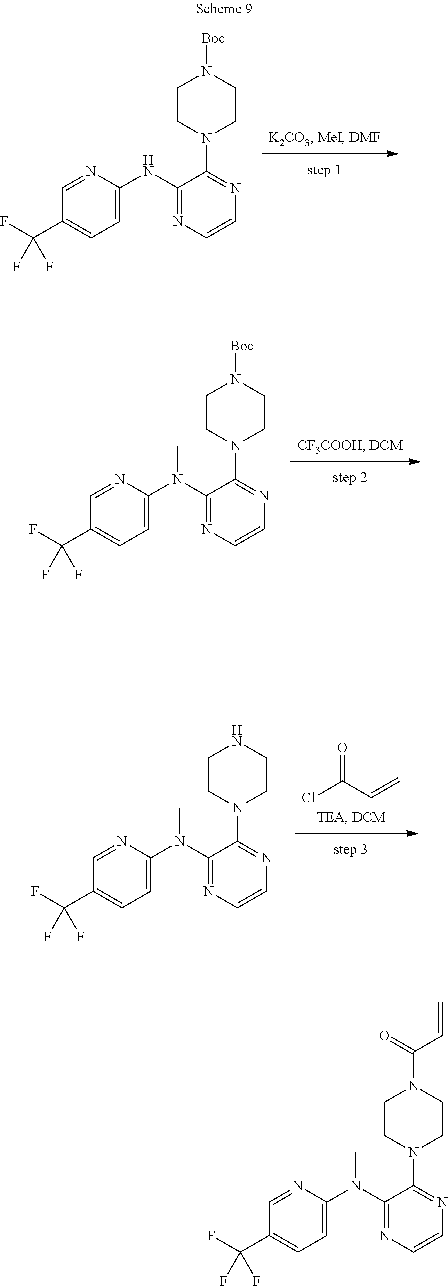

### Step 1: Synthesis of tert-butyl 4-(3-(methyl(5-(trifluoromethyl)pyridin-2-yl)amino)pyrazin-2-yl)piperazine-1-carboxylate

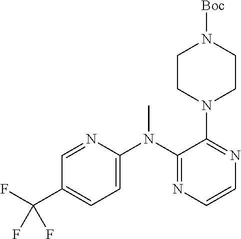 [SMILES: CN(c1ccc(C(F)(F)F)cn1)c1nccnc1N1CCN(C(=O)OC(C)(C)C)CC1]

A solution of tert-butyl 4-(3-{[5-(trifluoromethyl)pyridin-2-yl]amino}pyrazin-2-yl)piperazine-1-carboxylate (1 g, 2.356 mmol, 1 equiv), Mel (0.33 g, 2.356 mmol, 1 equiv) and K₂CO₃ (0.65 g, 4.712 mmol, 2 equiv) in DMF (10 mL) was stirred for 2 h at room temperature. The product was precipitated by the addition of water. The crude product was used in the next step directly without further purification.

### Step 2: Synthesis ofN-methyl-3-(piperazin-1-yl)-N-(5-(trifluoromethyl)pyridin-2-yl)pyrazin-2-amine

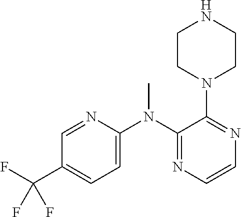 [SMILES: CN(c1ccc(C(F)(F)F)cn1)c1nccnc1N1CCNCC1]

A solution of tert-butyl 4-(3-{methyl[5-(trifluoromethyl)pyridin-2-yl]amino}pyrazin-2-yl)piperazine-1-carboxylate (300 mg, 0.684 mmol, 1 equiv) and TFA (1 mL, 13.463 mmol, 19.68 equiv) in DCM (4 mL) was stirred for 1 h at room temperature. The resulting mixture was concentrated under reduced pressure. The residue product was purified by reverse phase flash to afford N-methyl-3-(piperazin-1-yl)-N-[5-(trifluoromethyl)pyridin-2-yl]pyrazin-2-amine (200 mg, 86.39%) as a yellow solid.

### Step 3: Synthesis of 1-(4-(3-(methyl(5-(trifluoromethyl)pyridin-2-yl)amino)pyrazin-2-yl)piperazin-1-yl)prop-2-en-1-one

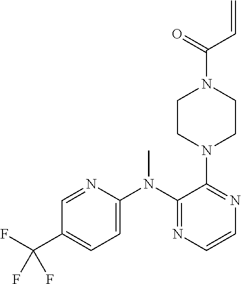 [SMILES: C=CC(=O)N1CCN(c2nccnc2N(C)c2ccc(C(F)(F)F)cn2)CC1]

A solution of N-methyl-3-(piperazin-1-yl)-N-[5-(trifluoromethyl)pyridin-2-yl]pyrazin-2-amine (200 mg, 0.591 mmol, 1 equiv) and acryloyl chloride (53.50 mg, 0.591 mmol, 1.00 equiv) in DCM (2 mL) was stirred for 1 h at room temperature. The resulting mixture was extracted with EtOAc (2×2 mL). The combined organic layers, dried over anhydrous Na₂SO₄. After filtration, the filtrate was concentrated under reduced pressure. The residue/crude product was purified by reverse phase flash (Column: Welch Xtimate C18 ExRS, 250 mm, 10 μm; Mobile Phase A: Water (0.05% NH₃·H₂O), Mobile Phase B: ACN; Flow rate: 90 mL/min; Gradient: 90% B to 90% B in 10 min, 90% B; Wave Length: 254 nm; RT₁(min): 7; Number Of Runs: 5) to afford 1-[4-(3-{methyl[5-(trifluoromethyl)pyridin-2-yl]amino}pyrazin-2-yl)piperazin-1-yl]prop-2-en-1-one (70 mg, 30.18%) as a white solid. ¹H NMR (400 MHz, DMSO-d₆): δ 8.60-8.54 (m, 1H), 8.21 (d, J=2.5 Hz, 1H), 8.04 (d, J=2.5 Hz, 1H), 7.86 (dd, J=9.0, 2.6 Hz, 1H), 6.81-6.69 (m, 2H), 6.09 (dd, J=16.7, 2.4 Hz, 1H), 5.67 (dd, J=10.5, 2.4 Hz, 1H), 3.47 (s, 3H), 3.22 (dd, J=6.4, 3.7 Hz, 4H).

### Example 43: Synthesis of 1-(4-(3-((5-(trifluoromethyl)pyridin-2-yl)amino)pyrazin-2-yl)-3,6-dihydropyridin-1(2H)-yl)prop-2-en-1-one

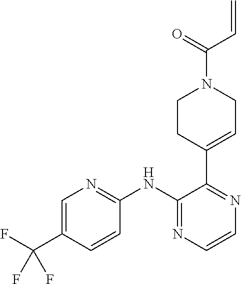 [SMILES: C=CC(=O)N1CC=C(c2nccnc2Nc2ccc(C(F)(F)F)cn2)CC1]

Following the general procedure in scheme 5, 3-chloro-N-(5-(trifluoromethyl)pyridin-2-yl)pyrazin-2-amine was used in step 1. Offered 1-(4-(3-((5-(trifluoromethyl)pyridin-2-yl)amino)pyrazin-2-yl)-3,6-dihydropyridin-1(2H)-yl)prop-2-en-1-one as white solid. (Positive, ES, m/z): 376.22. ¹H NMR (400 MHz, DMSO) δ 9.56 (d, J=12.2 Hz, 1H), 8.53 (s, 1H), 8.28 (dd, J=11.1, 2.7 Hz, 2H), 8.03 (dd, J=9.1, 2.6 Hz, 1H), 7.78 (dd, J=15.9, 8.8 Hz, 1H), 6.98-6.61 (m, 1H), 6.29-6.08 (m, 2H), 5.71 (ddd, J=10.3, 4.8, 2.4 Hz, 1H), 4.22-4.03 (m, 2H), 3.74 (dt, J=16.6, 5.6 Hz, 2H), 2.66-2.53 (m, 2H).

### Example 44: Synthesis of 1-(4-(3-((5-(trifluoromethyl)pyridin-2-yl)amino)pyrazin-2-yl)piperidin-1-yl)prop-2-en-1-one

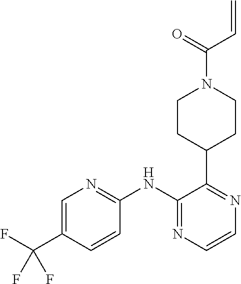 [SMILES: C=CC(=O)N1CCC(c2nccnc2Nc2ccc(C(F)(F)F)cn2)CC1]

Following the general procedure in scheme 7, tert-butyl 4-(3-((5-(trifluoromethyl)pyridin-2-yl)amino)pyrazin-2-yl)-3,6-dihydropyridine-1(2H)-carboxylate was used in step 1. Offered 1-(4-(3-((5-(trifluoromethyl)pyridin-2-yl)amino)pyrazin-2-yl)piperidin-1-yl)prop-2-en-1-one as white solid. (Positive, ES, m/z): 378.28. ¹H NMR (400 MHz, DMSO) δ 9.80 (s, 1H), 8.60 (dt, J=2.0, 1.0 Hz, 1H), 8.33-8.15 (m, 2H), 8.04 (dd, J=9.0, 2.6 Hz, 1H), 7.84 (d, J=8.9 Hz, 1H), 6.85 (dd, J=16.7, 10.5 Hz, 1H), 6.12 (dd, J=16.7, 2.5 Hz, 1H), 5.68 (dd, J=10.4, 2.5 Hz, 1H), 4.56 (d, J=12.9 Hz, 1H), 4.18 (d, J=13.7 Hz, 1H), 3.63-3.47 (m, 1H), 3.25-3.11 (m, 1H), 2.77 (t, J=12.5 Hz, 1H), 1.86 (d, J=12.8 Hz, 2H), 1.75-1.48 (m, 2H).

### Example 45: Synthesis of 1-(4-(3-((5-(trifluoromethyl)pyridin-2-yl)amino)pyrazin-2-yl)piperazin-1-yl)prop-2-en-1-one

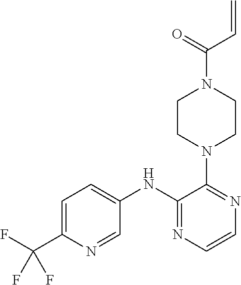 [SMILES: C=CC(=O)N1CCN(c2nccnc2Nc2ccc(C(F)(F)F)nc2)CC1]

Following the general procedure in scheme 1, 6-(trifluoromethyl)pyridin-3-amine was used in step 2. Offered 1-(4-(3-((5-(trifluoromethyl)pyridin-2-yl)amino)pyrazin-2-yl)piperazin-1-yl)prop-2-en-1-one as white solid. (Positive, ES, m/z): 379.19. ¹H NMR (400 MHz, DMSO) δ 9.03 (d, J=2.5 Hz, 1H), 8.88 (s, 1H), 8.39 (dd, J=8.7, 2.5 Hz, 1H), 7.99-7.86 (m, 2H), 7.83 (d, J=8.7 Hz, 1H), 6.86 (dd, J=16.7, 10.5 Hz, 1H), 6.16 (dd, J=16.7, 2.4 Hz, 1H), 5.73 (dd, J=10.4, 2.4 Hz, 1H), 3.92-3.69 (m, 4H), 3.19 (d, J=6.1 Hz, 4H).

### Example 46. Synthesis of 2-fluoro-1-(4-(3-((6-(trifluoromethyl)pyridin-3-yl)amino)pyrazin-2-yl)piperazin-1-yl)prop-2-en-1-one

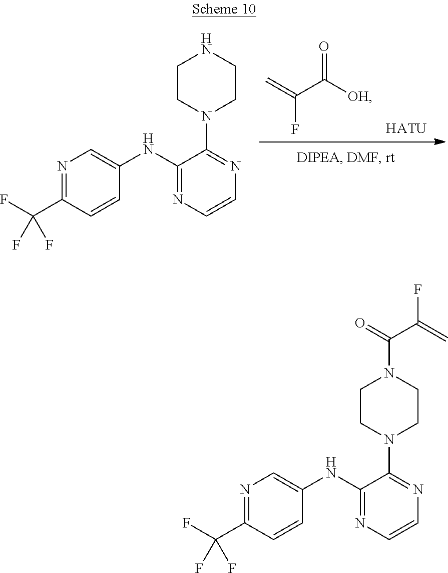

To a solution of 2-fluoroacrylic acid (0.032 g, 0.37 mmol, 1.2 equiv) in DMF (2 mL) was stirred at 0° C. for 5 min. Then added HATU (0.14 g, 0.37 mmol, 1.2 equiv) and DIPEA (153 mmL, 0.93 mmol, 3 equiv) dropwise, after stirred for another 5 min, the 3-(piperazin-1-yl)-N-(6-(trifluoromethyl)pyridin-3-yl)pyrazin-2-amine (0.1 g, 0.31 mmol, 1 equiv) was added. The reaction was continued at 0° C. for 1 hour, after which it was quenched with H₂O (50 mL), NaCl (sat. aq. soln., 20 mL), and EtOAc (20 mL). Aqueous layer was separated and extracted with EA (3×20 mL). Combined organic phases were dried over Na₂SO₄, concentrated in vacuo. Purification by flash column chromatography eluted with EA:DCM (2:3) to afford 2-fluoro-1-(4-(3-((6-(trifluoromethyl)pyridin-3-yl)amino)pyrazin-2-yl)piperazin-1-yl)prop-2-en-1-one (80 mg, 65.46% yield) as white solid. (Positive, ES, m/z): 397.06. ¹H NMR (400 MHz, DMSO) δ 9.03 (d, J=2.5 Hz, 1H), 8.88 (s, 1H), 8.39 (dd, J=8.7, 2.5 Hz, 1H), 7.95-7.87 (m, 2H), 7.84 (d, J=8.7 Hz, 1H), 5.41-5.13 (m, 2H), 3.78 (t, J=4.8 Hz, 4H), 3.23 (t, J=5.1 Hz, 4H).

### Example 47: Synthesis of 1-(4-(3-((6-(difluoromethyl)pyridin-3-yl)amino)pyrazin-2-yl)piperazin-1-yl)prop-2-en-1-one

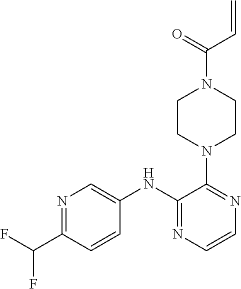 [SMILES: C=CC(=O)N1CCN(c2nccnc2Nc2ccc(C(F)F)nc2)CC1]

Following the general procedure in scheme 1, 6-(difluoromethyl)pyridin-3-amine was used in step 2. Offered 1-(4-(3-((6-(difluoromethyl)pyridin-3-yl)amino)pyrazin-2-yl)piperazin-1-yl)prop-2-en-1-one as white solid. (Positive, ES, m/z): 361.77. ¹H NMR (400 MHz, DMSO) δ 8.96 (d, J=2.5 Hz, 1H), 8.70 (s, 1H), 8.32 (dd, J=8.5, 2.6 Hz, 1H), 7.97-7.82 (m, 2H), 7.65 (d, J=8.6 Hz, 1H), 7.05-6.72 (m, 2H), 6.16 (dd, J=16.7, 2.4 Hz, 1H), 5.73 (dd, J=10.4, 2.4 Hz, 1H), 3.80 (d, J=13.1 Hz, 4H), 3.17 (s, 4H).

### Example 48: Synthesis of 1-(4-(3-((6-(trifluoromethoxy)pyridin-3-yl)amino)pyrazin-2-yl)piperazin-1-yl)prop-2-en-1-one

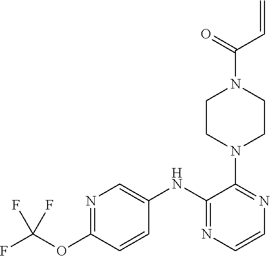 [SMILES: C=CC(=O)N1CCN(c2nccnc2Nc2ccc(OC(F)(F)F)nc2)CC1]

Following the general procedure in scheme 1, 6-(trifluoromethoxy)pyridin-3-amine was used in step 2. Offered 1-(4-(3-((6-(trifluoromethoxy)pyridin-3-yl)amino)pyrazin-2-yl)piperazin-1-yl)prop-2-en-1-one as white solid. (Positive, ES, m/z): 395.01. ¹H NMR (400 MHz, DMSO) δ 8.65 (d, J=2.8 Hz, 1H), 8.58 (s, 1H), 8.30 (dd, J=8.8, 2.8 Hz, 1H), 7.82 (q, J=2.8 Hz, 2H), 7.30 (d, J=8.8 Hz, 1H), 6.87 (dd, J=16.7, 10.4 Hz, 1H), 6.16 (dd, J=16.7, 2.4 Hz, 1H), 5.73 (dd, J=10.4, 2.4 Hz, 1H), 3.80 (s, 4H), 3.16 (s, 4H).

### Example 49: Synthesis of 1-(4-(3-((6-(difluoromethoxy)pyridin-3-yl)amino)pyrazin-2-yl)piperazin-1-yl)prop-2-en-1-one

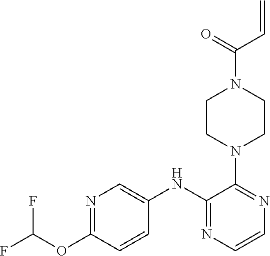 [SMILES: C=CC(=O)N1CCN(c2nccnc2Nc2ccc(OC(F)F)nc2)CC1]

Following the general procedure in scheme 1, 6-(difluoromethoxy)pyridin-3-amine was used in step 2. Offered 1-(4-(3-((6-(difluoromethoxy)pyridin-3-yl)amino)pyrazin-2-yl)piperazin-1-yl)prop-2-en-1-one as white solid. (Positive, ES, m/z): 377.63. ¹H NMR (400 MHz, DMSO) δ 8.53 (d, J=2.7 Hz, 1H), 8.41 (s, 1H), 8.18 (dd, J=8.8, 2.8 Hz, 1H), 7.87-7.43 (m, 3H), 7.09 (d, J=8.8 Hz, 1H), 6.87 (dd, J=16.7, 10.4 Hz, 1H), 6.16 (dd, J=16.7, 2.4 Hz, 1H), 5.73 (dd, J=10.4, 2.4 Hz, 1H), 3.80 (s, 4H), 3.15 (d, J=6.1 Hz, 4H).

### Example 50: Synthesis of 1-(4-(3-((5-fluoro-6-(trifluoromethyl)pyridin-3-yl)amino)pyrazin-2-yl)piperazin-1-yl)prop-2-en-1-one

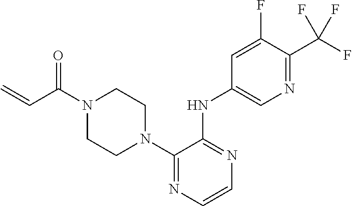 [SMILES: C=CC(=O)N1CCN(c2nccnc2Nc2cnc(C(F)(F)F)c(F)c2)CC1]

Following the general procedure in scheme 1, 5-fluoro-6-(trifluoromethyl)pyridin-3-amine was used in step 2. Offered 1-(4-(3-((5-fluoro-6-(trifluoromethyl)pyridin-3-yl)amino)pyrazin-2-yl)piperazin-1-yl)prop-2-en-1-one as white solid. (Positive, ES, m/z): 397.37. ¹H NMR (400 MHz, DMSO) δ 9.20 (d, J=7.4 Hz, 1H), 8.84 (d, J=9.0 Hz, 1H), 8.42 (d, J=13.3 Hz, 1H), 7.96 (d, J=7.8 Hz, 2H), 6.85 (d, J=14.0 Hz, 1H), 6.16 (d, J=10.8 Hz, 1H), 5.73 (d, J=11.4 Hz, 1H), 3.78 (s, 4H), 3.19 (s, 4H).

### Example 51: Synthesis of 2-fluoro-1-(4-(3-((5-fluoro-6-(trifluoromethyl)pyridin-3-yl)amino)pyrazin-2-yl)piperazin-1-yl)prop-2-en-1-one

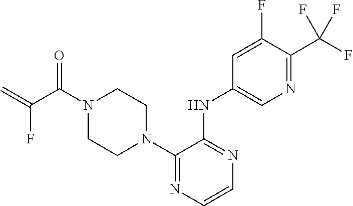 [SMILES: C=C(F)C(=O)N1CCN(c2nccnc2Nc2cnc(C(F)(F)F)c(F)c2)CC1]

Following the general procedure in scheme 10, N-(5-fluoro-6-(trifluoromethyl)pyridin-3-yl)-3-(piperazin-1-yl)pyrazin-2-amine was used to afford 2-fluoro-1-(4-(3-((5-fluoro-6-(trifluoromethyl)pyridin-3-yl)amino)pyrazin-2-yl)piperazin-1-yl)prop-2-en-1-one as white solid. (Positive, ES, m/z): 397.37. ¹H NMR (400 MHz, DMSO) δ 9.20 (s, 1H), 8.85 (s, 1H), 8.42 (d, J=13.6 Hz, 1H), 7.97 (d, J=8.4 Hz, 2H), 5.39-5.14 (m, 2H), 3.76 (s, 4H), 3.23 (s, 4H).

### Example 52: Synthesis of 1-(4-(3-((2-(trifluoromethyl)pyrimidin-5-yl)amino)pyrazin-2-yl)piperazin-1-yl)prop-2-en-1-one

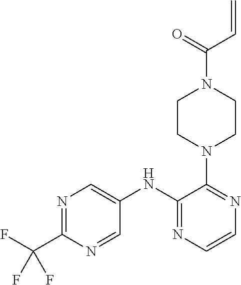 [SMILES: C=CC(=O)N1CCN(c2nccnc2Nc2cnc(C(F)(F)F)nc2)CC1]

Following the general procedure in scheme 1, 2-(trifluoromethyl)pyrimidin-5-amine was used in step 2. Offered 1-(4-(3-((2-(trifluoromethyl)pyrimidin-5-yl)amino)pyrazin-2-yl)piperazin-1-yl)prop-2-en-1-one as white solid. (Positive, ES, m/z): 380.18. ¹H NMR (400 MHz, DMSO) δ 9.34 (s, 2H), 9.10 (s, 1H), 7.99-7.87 (m, 2H), 6.87 (dd, =16.7, 10.4 Hz, 1H), 6.16 (dd, J=16.7, 2.4 Hz, 1H), 5.80-5.65 (m, 1H), 3.79 (d, J=11.8 Hz, 4H), 3.21 (d, J=6.4 Hz, 4H).

### Example 53: Synthesis of (S)-1-(2-methyl-4-(3-((6-(trifluoromethyl)pyridin-3-yl)amino)pyrazin-2-yl)piperazin-1-yl)prop-2-en-1-one

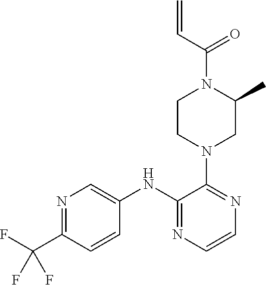 [SMILES: C=CC(=O)N1CCN(c2nccnc2Nc2ccc(C(F)(F)F)nc2)C[C@@H]1C]

Following the general procedure in scheme 1, tert-butyl (S)-2-methylpiperazine-1-carboxylate was used in step 1 and 6-(trifluoromethyl)pyridin-3-amine was used in step 2. Offered (S)-1-(2-methyl-4-(3-((6-(trifluoromethyl)pyridin-3-yl)amino)pyrazin-2-yl)piperazin-1-yl)prop-2-en-1-one as white solid. (Positive, ES, m/z): 393.26. ¹H NMR (400 MHz, DMSO) δ 8.96 (d, J=2.5 Hz, 1H), 8.60 (s, 1H), 8.32 (dd, J=8.7, 2.5 Hz, 1H), 7.91 (s, 2H), 7.85 (d, J=8.6 Hz, 1H), 6.83 (dd, J=16.7, 10.4 Hz, 1H), 6.15 (dd, J=16.6, 2.4 Hz, 1H), 5.72 (dd, J=10.5, 2.4 Hz, 1H), 4.88-3.97 (m, 2H), 3.76-3.60 (m, 1H), 3.62-3.35 (m, 2H), 3.01 (d, J=12.8 Hz, 1H), 2.70 (s, 1H), 1.33 (d, J=6.7 Hz, 3H).

### Example 54: Synthesis of (R)-1-(2-methyl-4-(3-((6-(trifluoromethyl)pyridin-3-yl)amino)pyrazin-2-yl)piperazin-1-yl)prop-2-en-1-one

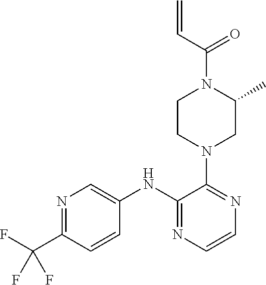 [SMILES: C=CC(=O)N1CCN(c2nccnc2Nc2ccc(C(F)(F)F)nc2)C[C@H]1C]

Following the general procedure in scheme 1, tert-butyl (R)-2-methylpiperazine-1-carboxylate was used in step 1 and 6-(trifluoromethyl)pyridin-3-amine was used in step 2. Offered (R)-1-(2-methyl-4-(3-((6-(trifluoromethyl)pyridin-3-yl)amino)pyrazin-2-yl)piperazin-1-yl)prop-2-en-1-one as white solid. (Positive, ES, m/z): 393.29. ¹H NMR (400 MHz, DMSO) δ 8.96 (d, J=2.5 Hz, 1H), 8.60 (s, 1H), 8.32 (dd, J=8.7, 2.6 Hz, 1H), 7.91 (s, 2H), 7.85 (d, J=8.7 Hz, 1H), 6.83 (dd, J=16.7, 10.5 Hz, 1H), 6.15 (dd, J=16.7, 2.4 Hz, 1H), 5.72 (dd, J=10.5, 2.4 Hz, 1H), 3.66 (d, J=12.1 Hz, 2H), 3.56-3.36 (m, 2H), 3.02 (s, 1H), 2.69 (s, 1H), 1.46-1.22 (m, 3H).

### Example 55: Synthesis of 1-(4-(3-((6-(trifluoromethyl)pyridin-3-yl)amino)pyrazin-2-yl)-3, 6-dihydropyridin-1(2H)-yl)prop-2-en-1-one

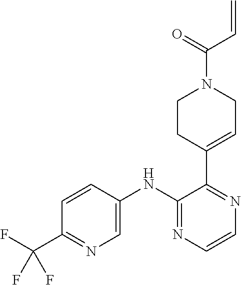 [SMILES: C=CC(=O)N1CC=C(c2nccnc2Nc2ccc(C(F)(F)F)nc2)CC1]

Following the general procedure in scheme 6, 3-chloro-N-(6-(trifluoromethyl)pyridin-2-yl)pyrazin-2-amine was used in step 1. Offered 1-(4-(3-((6-(trifluoromethyl)pyridin-3-yl)amino)pyrazin-2-yl)-3,6-dihydropyridin-1(2H)-yl)prop-2-en-1-one as white solid. (Positive, ES, m/z): 376.29. ¹H NMR (400 MHz, DMSO) δ 9.08-8.89 (m, 2H), 8.30 (d, J=8.7 Hz, 1H), 8.16 (dt, J=14.0, 2.4 Hz, 2H), 7.82 (d, J=8.6 Hz, 1H), 7.00-6.72 (m, 1H), 6.37 (d, J=14.9 Hz, 1H), 6.19 (ddd, J=16.7, 9.6, 2.4 Hz, 1H), 5.75 (td, J=9.6, 2.4 Hz, 1H), 4.39-4.17 (m, 2H), 3.87-3.74 (m, 2H), 2.70-2.55 (m, 2H).

### Example 56: Synthesis of 2-fluoro-1-(4-(3-((6-(trifluoromethyl)pyridin-3-yl)amino)pyrazin-2-yl)-3, 6-dihydropyridin-1(2H)-yl)prop-2-en-1-one

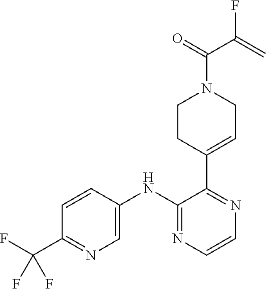 [SMILES: C=C(F)C(=O)N1CC=C(c2nccnc2Nc2ccc(C(F)(F)F)nc2)CC1]

Following the general procedure in scheme 10, 3-(1,2,3,6-tetrahydropyridin-4-yl)-N-(6-(trifluoromethyl)pyridin-3-yl)pyrazin-2-amine was used in step 1. Offered 2-fluoro-1-(4-(3-((6-(trifluoromethyl)pyridin-3-yl)amino)pyrazin-2-yl)-3,6-dihydropyridin-1(2H)-yl)prop-2-en-1-one as white solid. (Positive, ES, m/z): 394.32. ¹H NMR (400 MHz, DMSO) δ 9.04-8.88 (m, 2H), 8.30 (d, J=8.3 Hz, 1H), 8.22-7.99 (m, 2H), 7.82 (d, J=8.7 Hz, 1H), 6.37 (s, 1H), 5.44 5.17 (m, 2H), 4.38-4.15 (m, 2H), 3.79 (t, J=5.8 Hz, 2H), 2.67 (s, 2H).

### Example 57: Synthesis of 1-(4-(3-((6-(trifluoromethoxy)pyridin-3-yl)amino)pyrazin-2-yl)-3, 6-dihydropyridin-1(2H)-yl)prop-2-en-1-one

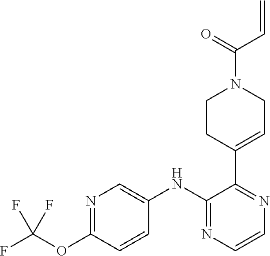 [SMILES: C=CC(=O)N1CC=C(c2nccnc2Nc2ccc(OC(F)(F)F)nc2)CC1]

Following the general procedure in scheme 6, 3-chloro-N-(6-(trifluoromethoxy)pyridin-3-yl)pyrazin-2-amine was used in step 1. Offered 1-(4-(3-((6-(trifluoromethoxy)pyridin-3-yl)amino)pyrazin-2-yl)-3,6-dihydropyridin-1(2H)-yl)prop-2-en-1-one as white solid. (Positive, ES, m/z): 392.76. ¹H NMR (400 MHz, DMSO) δ 8.70 (d, J=4.0 Hz, 1H), 8.55 (s, 1H), 8.21 (d, J=8.5 Hz, 1H), 8.06 (d, J=12.2 Hz, 2H), 7.28 (d, J=8.8 Hz, 1H), 6.99-6.75 (m, 1H), 6.35 (d, J=14.0 Hz, 1H), 6.18 (dd, J=17.1, 9.1 Hz, 1H), 5.75 (t, J=9.4 Hz, 1H), 4.39-4.18 (m, 2H), 3.81 (d, J=8.1 Hz, 2H), 2.62 (s, 2H).

### Example 58: Synthesis of 2-fluoro-1-(4-(3-((6-(trifluoromethoxy)pyridin-3-yl)amino)pyrazin-2-yl)-3, 6-dihydropyridin-1(2H)-yl)prop-2-en-1-one

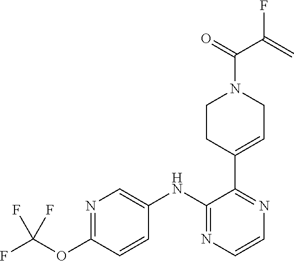 [SMILES: C=C(F)C(=O)N1CC=C(c2nccnc2Nc2ccc(OC(F)(F)F)nc2)CC1]

Following the general procedure in scheme 10, 3-(1,2,3,6-tetrahydropyridin-4-yl)-N-(6-(trifluoromethoxy)pyridin-3-yl)pyrazin-2-amine was used in step 1. Offered 2-fluoro-1-(4-(3-((6-(trifluoromethoxy)pyridin-3-yl)amino)pyrazin-2-yl)-3,6-dihydropyridin-1(2H)-yl)prop-2-en-1-one as white solid. (Positive, ES, m/z): 410.78. ¹H NMR (400 MHz, DMSO) δ 8.71 (s, 1H), 8.56 (dd, 1=2.7, 1.3 Hz, 1H), 8.20 (dd, =8.8, 2.6 Hz, 1H), 8.11-7.98 (m, 2H), 7.28 (d, I=8.8 Hz, 1H), 6.36 (s, 1H), 5.45-5.13 (m, 2H), 4.36-4.15 (m, 2H), 3.78 (t, J=5.7 Hz, 2H), 2.65 (s, 2H).

### Example 59: Synthesis of 1-(4-(3-((2-(trifluoromethyl)pyrimidin-5-yl)amino)pyrazin-2-yl)-3, 6-dihydropyridin-1(2H)-yl)prop-2-en-1-one

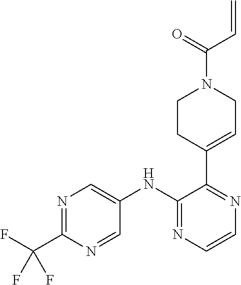 [SMILES: C=CC(=O)N1CC=C(c2nccnc2Nc2cnc(C(F)(F)F)nc2)CC1]

Following the general procedure in scheme 6, N-(3-chloropyrazin-2-yl)-2-(trifluoromethyl)pyrimidin-5-amine was used in step 1. Offered 1-(4-(3-((2-(trifluoromethyl)pyrimidin-5-yl)amino)pyrazin-2-yl)-3, 6-dihydropyridin-1(2H)-yl)prop-2-en-1-one as white solid. (Positive, ES, m/z): 410.78. ¹H NMR (400 MHz, DMSO) δ 9.41-9.10 (m, 3H), 8.29-8.01 (m, 2H), 7.01-6.78 (m, 1H), 6.41 (d, J=12.7 Hz, 1H), 6.19 (dd, J=16.5, 10.4 Hz, 1H), 5.75 (dd, J=11.2, 8.8 Hz, 1H), 4.38-4.19 (m, 2H), 3.91-3.74 (m, 2H), 3.32 (s, 1H), 2.68-2.57 (m, 2H).

### Example 60: Synthesis of 2-fluoro-1-(4-(3-((2-(trifluoromethyl)pyrimidin-5-yl)amino)pyrazin-2-yl)-3,6-dihydropyridin-1(2H)-yl)prop-2-en-1-one

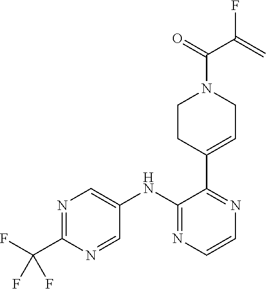 [SMILES: C=C(F)C(=O)N1CC=C(c2nccnc2Nc2cnc(C(F)(F)F)nc2)CC1]

Following the general procedure in scheme 10, N-(3-(1,2,3,6-tetrahydropyridin-4-yl)pyrazin-2-yl)-2-(trifluoromethyl)pyrimidin-5-amine was used in step 1. Offered 2-fluoro-1-(4-(3-((2-(trifluoromethyl)pyrimidin-5-yl)amino)pyrazin-2-yl)-3,6-dihydropyridin-1(2H)-yl)prop-2-en-1-one as white solid. (Positive, ES, m/z): 395.08. ¹H NMR (400 MHz, DMSO) δ 9.40-9.10 (m, 3H), 8.33-8.09 (m, 2H), 6.42 (s, 1H), 5.44-5.13 (m, 2H), 4.40-4.17 (m, 2H), 3.80 (t, J=5.6 Hz, 2H), 3.32 (s, 1H), 2.68 (s, 2H).

### Example 61: Synthesis of 1-(4-(3-((6-(trifluoromethyl)pyridin-3-yl)amino)pyrazin-2-yl)piperidin-1-yl)prop-2-en-1-one

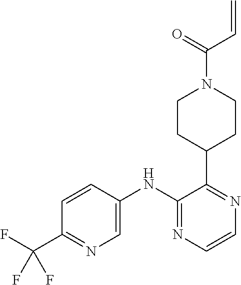 [SMILES: C=CC(=O)N1CCC(c2nccnc2Nc2ccc(C(F)(F)F)nc2)CC1]

Following the general procedure in scheme 7, tert-butyl 4-(3-((6-(trifluoromethyl)pyridin-2-yl)amino)pyrazin-2-yl)-3,6-dihydropyridine-1(2H)-carboxylate was used in step 1. Offered 1-(4-(3-((6-(trifluoromethyl)pyridin-3-yl)amino)pyrazin-2-yl)piperidin-1-yl)prop-2-en-1-one as white solid. (Positive, ES, m/z): 378.27. ¹H NMR (400 MHz, DMSO) δ 9.05 (s, 1H), 8.98 (d, J=2.6 Hz, 1H), 8.33 (dd, J=8.7, 2.5 Hz, 1H), 8.21-8.02 (m, 2H), 7.84 (d, J=8.6 Hz, 1H), 6.87 (dd, J=16.7, 10.5 Hz, 1H), 6.13 (dd, J=16.7, 2.5 Hz, 1H), 5.69 (dd, J=10.5, 2.5 Hz, 1H), 4.60 (d, J=12.9 Hz, 1H), 4.22 (d, J=13.5 Hz, 1H), 3.51 (dtd, J=11.6, 7.7, 4.0 Hz, 1H), 3.26 (t, J=13.2 Hz, 1H), 2.94-2.74 (m, 1H), 1.90 (d, J=13.0 Hz, 2H), 1.76-1.50 (m, 2H).

### Example 62: Synthesis of 1-(3-((3-((6-(trifluoromethyl)pyridin-3-yl)amino)pyrazin-2-yl)amino)azetidin-1-yl)prop-2-en-1-one

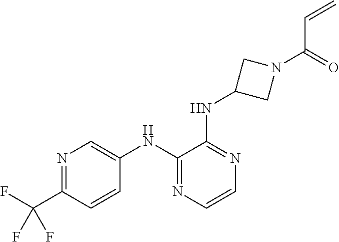 [SMILES: C=CC(=O)N1CC(Nc2nccnc2Nc2ccc(C(F)(F)F)nc2)C1]

Following the general procedure in scheme 1, tert-butyl 3-aminoazetidine-1-carboxylate was used in step 1 and 6-(trifluoromethyl)pyridin-3-amine was used in step 2. Offered 1-(3-((3-((6-(trifluoromethyl)pyridin-3-yl)amino)pyrazin-2-yl)amino)azetidin-1-yl)prop-2-en-1-one as white solid. (Positive, ES, m/z): 365.2. ¹H NMR (400 MHz, DMSO) δ 8.87 (d, J=8.4 Hz, 2H), 8.41 (d, J=11.1 Hz, 1H), 7.82 (q, J=6.3 Hz, 1H), 7.71-7.47 (m, 2H), 7.28 (s, 1H), 6.36 (d, J=14.6 Hz, 1H), 6.14 (d, J=14.0 Hz, 1H), 5.70 (d, J=10.8 Hz, 1H), 4.62 (d, J=9.9 Hz, 2H), 4.32 (s, 1H), 4.07 (s, 1H), 3.91 (s, 1H).

### Example 63: Synthesis of 2-fluoro-1-(3-((3-((6-(trifluoromethyl)pyridin-3-yl)amino)pyrazin-2-yl)amino)azetidin-1-yl)prop-2-en-1-one

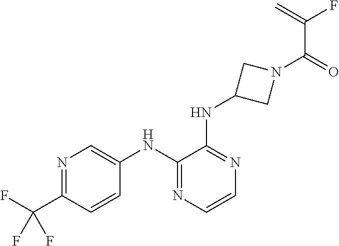 [SMILES: C=C(F)C(=O)N1CC(Nc2nccnc2Nc2ccc(C(F)(F)F)nc2)C1]

Following the general procedure in scheme 10, N²-(azetidin-3-yl)-N³-(6-(trifluoromethyl)pyridin-3-yl)pyrazine-2,3-diamine was used. Offered 2-fluoro-1-(3-((3-((6-(trifluoromethyl)pyridin-3-yl)amino)pyrazin-2-yl)amino)azetidin-1-yl)prop-2-en-1-one as white solid. (Positive, ES, m/z): 383.31. ¹H NMR (400 MHz, DMSO) δ 8.97-8.80 (m, 2H), 8.40 (dd, J=8.7, 2.6 Hz, 1H), 7.83 (d, J=8.7 Hz, 1H), 7.66 (d, J=2.9 Hz, 1H), 7.54 (d, J=3.0 Hz, 1H), 7.29 (d, J=5.5 Hz, 1H), 5.61-5.41 (m, 1H), 5.33 (dd, J=16.6, 3.6 Hz, 1H), 4.88-4.61 (m, 2H), 4.46-4.33 (m, 1H), 4.29-4.17 (m, 1H), 3.95 (dd, J=10.7, 4.8 Hz, 1H).

### Example 64: Synthesis of 2-fluoro-1-(3-((3-((6-(trifluoromethoxy)pyridin-3-yl)amino)pyrazin-2-yl)amino)azetidin-1-yl)prop-2-en-1-one

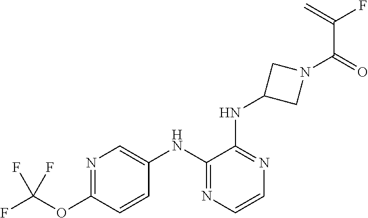 [SMILES: C=C(F)C(=O)N1CC(Nc2nccnc2Nc2ccc(OC(F)(F)F)nc2)C1]

Following the general procedure in scheme 10, N²-(azetidin-3-yl)-N³-(6-(trifluoromethoxy)pyridin-3-yl)pyrazine-2,3-diamine was used. Offered 2-fluoro-1-(3-((3-((6-(trifluoromethoxy)pyridin-3-yl)amino)pyrazin-2-yl)amino)azetidin-1-yl)prop-2-en-1-one as white solid. (Positive, ES, m/z): 399.2. ¹H NMR (400 MHz, DMSO) δ 8.64-8.51 (m, 2H), 8.29 (dd, J=8.9, 2.8 Hz, 1H), 7.58 (d, J=3.0 Hz, 1H), 7.47 (d, J=3.0 Hz, 1H), 7.29 (d, J=8.8 Hz, 1H), 7.19 (d, J=5.5 Hz, 1H), 5.57-5.41 (m, 1H), 5.32 (dd, J=16.6, 3.6 Hz, 1H), 4.85-4.62 (m, 2H), 4.44-4.33 (m, 1H), 4.23 (s, 1H), 4.01-3.88 (m, 1H).

### Example 65: Synthesis of 2-fluoro-1-(3-((3-((6-(trifluoromethyl)pyridin-3-yl)amino)pyrazin-2-yl)oxy)azetidin-1-yl)prop-2-en-1-one

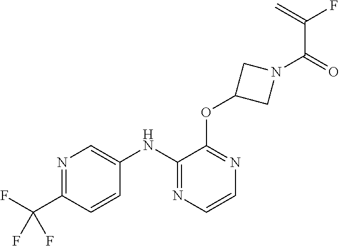 [SMILES: C=C(F)C(=O)N1CC(Oc2nccnc2Nc2ccc(C(F)(F)F)nc2)C1]

Following the general procedure in scheme 10, 3-(azetidin-3-yloxy)-N-(6-(trifluoromethyl)pyridin-3-yl)pyrazin-2-amine was used. Offered 2-fluoro-1-(3-((3-((6-(trifluoromethyl)pyridin-3-yl)amino)pyrazin-2-yl)oxy)azetidin-1-yl)prop-2-en-1-one as white solid. (Positive, ES, m/z): 384.41. ¹H NMR (400 MHz, DMSO) δ 9.33 (s, 1H), 9.17 (dd, J=5.6, 2.5 Hz, 1H), 8.81-8.54 (m, 1H), 7.93-7.62 (m, 2H), 6.74-6.53 (m, 1H), 5.77-5.28 (m, 3H), 4.85 (s, 1H), 4.65-4.41 (m, 1H), 4.29-4.11 (m, 1H), 3.98 (d, J=14.4 Hz, 1H), 3.77 (q, J=17.3 Hz, 1H).

### Example 66: Synthesis of 1-(4-(3-((4,4-difluorocyclohexyl)amino)pyrazin-2-yl)piperazin-1-yl)prop-2-en-1-one

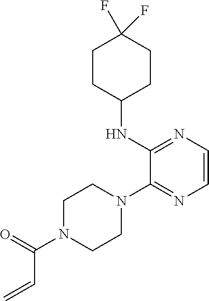 [SMILES: C=CC(=O)N1CCN(c2nccnc2NC2CCC(F)(F)CC2)CC1]

Following the general procedure in scheme 1, 4,4-difluorocyclohexan-1-amine was used in step 2. Offered 1-(4-(3-((4,4-difluorocyclohexyl)amino)pyrazin-2-yl)piperazin-1-yl)prop-2-en-1-one as white solid. (Positive, ES, m/z): 352.28. ¹H NMR (400 MHz, DMSO) δ 7.70 (d, J=2.9 Hz, 1H), 7.47 (d, J=2.9 Hz, 1H), 6.86 (dd, J=16.7, 10.5 Hz, 1H), 6.15 (dd, J=16.7, 2.4 Hz, 1H), 5.92 (d, J=7.8 Hz, 1H), 5.71 (dd, J=10.4, 2.4 Hz, 1H), 4.03 (s, 1H), 3.76 (d, J=14.1 Hz, 4H), 3.02 (s, 4H), 2.16-1.84 (m, 6H), 1.74 (t, J=11.3 Hz, 2H).

### Example 67: Synthesis of 1-(4-(3-((4-(trifluoromethyl)bicyclo[1.1.1]pentan-2-yl)amino)pyrazin-2-yl)piperazin-1-yl)prop-2-en-1-one

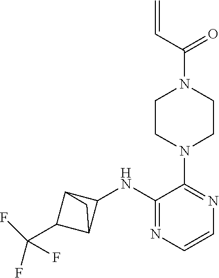 [SMILES: C=CC(=O)N1CCN(c2nccnc2NC2C3CC2C3C(F)(F)F)CC1]

Following the general procedure in scheme 1, 4-(trifluoromethyl)bicyclo[1.1.1]pentan-2-amine was used in step 2. Offered 1-(4-(3-((4-(trifluoromethyl)bicyclo[1.1.1]pentan-2-yl)amino)pyrazin-2-yl)piperazin-1-yl)prop-2-en-1-one as white solid. (Positive, ES, m/z): 368.02. ¹H NMR (400 MHz, DMSO) δ 7.76 (d, J=2.9 Hz, 1H), 7.57 (d, J=2.8 Hz, 1H), 7.05 (s, 1H), 6.85 (dd, J=16.7, 10.4 Hz, 1H), 6.15 (dd, J=16.7, 2.4 Hz, 1H), 5.72 (dd, J=10.4, 2.4 Hz, 1H), 3.75 (d, J=12.1 Hz, 4H), 3.00 (s, 4H), 2.37 (s, 6H).

### Example 68: Synthesis of 2-fluoro-1-(4-(3-((4-(trifluoromethyl)bicyclo[1.1.1]pentan-2-yl)amino)pyrazin-2-yl)piperazin-1-yl)prop-2-en-1-one

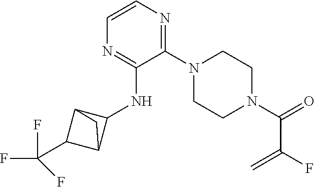 [SMILES: C=C(F)C(=O)N1CCN(c2nccnc2NC2C3CC2C3C(F)(F)F)CC1]

Following the general procedure in scheme 10, 3-(piperazin-1-yl)-N-(4-(trifluoromethyl)bicyclo[1.1.1]pentan-2-yl)pyrazin-2-amine was used. Offered 2-fluoro-1-(4-(3-((4-(trifluoromethyl)bicyclo[1.1.1]pentan-2-yl)amino)pyrazin-2-yl)piperazin-1-yl)prop-2-en-1-one as white solid. (Positive, ES, m/z): 386.66. ¹H NMR (400 MHz, DMSO) δ 7.77 (d, J=2.8 Hz, 1H), 7.57 (d, J=2.9 Hz, 1H), 7.07 (s, 1H), 5.36-5.13 (m, 2H), 3.74 (t, J=5.0 Hz, 4H), 3.04 (t, J=5.1 Hz, 4H), 2.37 (s, 6H).

### Example 69. Synthesis of 1-(4-(3-(4-(trifluoromethyl)phenoxy)pyrazin-2-yl)piperazin-1-yl)prop-2-en-1-one

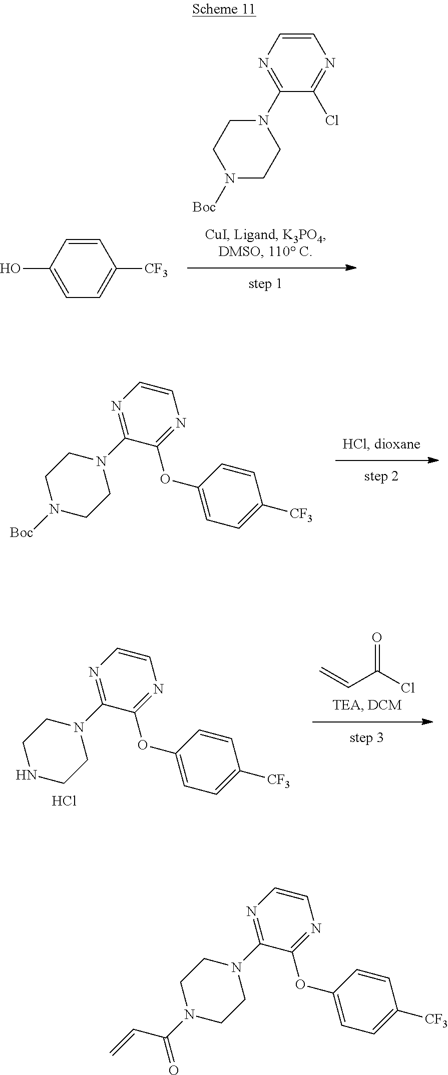

### Step 1: Synthesis of tert-butyl 4-{3-[4-(trifluoromethyl)phenoxy]pyrazin-2-yl}piperazine-1-carboxylate

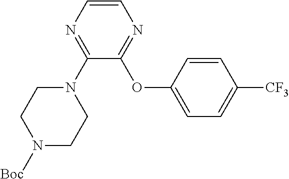 [SMILES: CC(C)(C)OC(=O)N1CCN(c2nccnc2Oc2ccc(C(F)(F)F)cc2)CC1]

Into a 40 mL vial were added tert-butyl 4-(3-chloropyrazin-2-yl) piperazine-1-carboxylate (500 mg, 1.32 mmol, 1.00 equiv), 4-(trifluoromethyl)phenol (1069.73 mg, 6.60 mmol, 5.00 equiv), CuI (25 mg, 0.13 mmol, 0.10 equiv), K₃PO₄ (840.41 mg, 3.96 mmol, 3.00 equiv), N1,N2-bis(2-thienylmethyl)-Ethanediamide (25 mg, 0.08 mmol, 0.07 equiv) and DMSO (10 mL) at room temperature. The resulting mixture was stirred for 8 h at 110° C. under nitrogen atmosphere. The resulting mixture was extracted with EtOAc (3×50 mL). The combined organic layers were washed with brine (1×50 mL), dried over anhydrous Na₂SO₄. After filtration, the filtrate was concentrated under reduced pressure. The residue was purified by silica gel column chromatography, eluted with PE/EA (2:1) to afford tert-butyl 4-{3-[4-(trifluoromethyl)phenoxy]pyrazin-2-yl}piperazine-1-carboxylate(250 mg 18.4%) as a white solid. LCMS ES, m/z: [M+H]⁺=425

### Step 2: Synthesis of 2-(piperazin-1-yl)-3-(4-(trifluoromethyl)phenoxy)pyrazine (HCl salt)

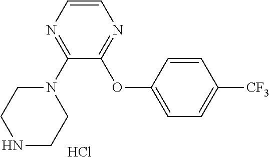 [SMILES: FC(F)(F)c1ccc(Oc2nccnc2N2CCNCC2)cc1 ; Cl]

A solution of tert-butyl 4-[3-(4-tert-butylphenoxy)pyrazin-2-yl]piperazine-1-carboxylate (230 mg, 0.55 mmol, 1.00 equiv) and HCl(4M in dioxane) (1 mL, 32.91 mmol, 59.03 equiv) in CH₂Cl₂ (3.0 mL)was stirred for 2 h at 0° C. under nitrogen atmosphere. The resulting mixture was concentrated under reduced pressure. The crude product was used in next step directly without further purification. LCMS ES, m/z: [M+H]⁺=325

### Step 3: Synthesis of 1-(4-(3-(4-(trifluoromethyl)phenoxy)pyrazin-2-yl)piperazin-1-yl)prop-2-en-1-one

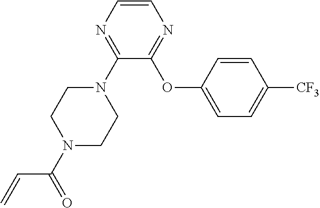 [SMILES: C=CC(=O)N1CCN(c2nccnc2Oc2ccc(C(F)(F)F)cc2)CC1]

A solution of 2-(4-tert-butylphenoxy)-3-(piperazin-1-yl)pyrazine (226.5 mg, 0.725 mmol, 1.00 equiv) in DCM (4.0 mL) was treated with TEA (212.21 mg, 2.09 mmol, 2.89 equiv) for 5 min at 0° C. under nitrogen atmosphere followed by the addition of acryloyl chloride (66.06 mg, 0.73 mmol, 1.01 equiv) dropwise at 0° C. The resulting mixture was concentrated and purified by reverse phase flash with the following conditions (Acetonitrile: Water/0.05% ammonia water 25%-67% 18 min) to afford 1-(4-(3-(4-(trifluoromethyl)phenoxy)pyrazin-2-yl)piperazin-1-yl)prop-2-en-1-one (220 mg, 82.81%) as a white solid. LCMS (ES, m/z: [M+H]⁺=379. ¹H NMR (400 MHz, DMSO-d₆): δ 8.02-7.98 (m, 1H), 7.85-7.78 (m, 2H), 7.63-7.58 (m, 1H), 7.48-7.40 (m, 2H), 6.89-6.79 (m, 1H), 6.20-6.11 (m, 1H), 5.75-5.68 (m, 1H), 3.80-3.63 (m, 4H), 3.63-3.48 (m, 4H).

### Example 70: Synthesis of 1-(4-(3-(4-methoxyphenoxy)pyrazin-2-yl)piperazin-1-yl)prop-2-en-1-one

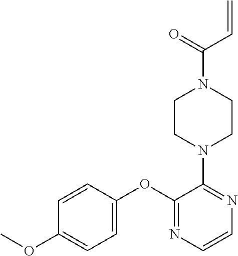 [SMILES: C=CC(=O)N1CCN(c2nccnc2Oc2ccc(OC)cc2)CC1]

Following the general procedure in scheme 11, 4-methoxyphenol was used in step 1. Offered 1-(4-(3-(4-methoxyphenoxy)pyrazin-2-yl)piperazin-1-yl)prop-2-en-1-one as white solid. (Positive, ES, m/z): 341.23. ¹H NMR (400 MHz, DMSO) δ 7.90 (d, J=2.8 Hz, 1H), 7.55 (d, J=2.7 Hz, 1H), 7.18-7.05 (m, 2H), 7.04-6.92 (m, 2H), 6.86 (dd, J=16.7, 10.4 Hz, 1H), 6.16 (dd, J=16.7, 2.4 Hz, 1H), 5.73 (dd, J=10.4, 2.3 Hz, 1H), 3.78 (s, 3H), 3.77-3.68 (m, 4H), 3.63-3.48 (m, 4H).

### Example 71: Synthesis of 1-(4-(3-(4-(difluoromethoxy)phenoxy)pyrazin-2-yl)piperazin-1-yl)prop-2-en-1-one

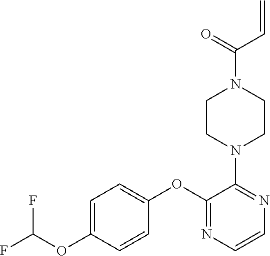 [SMILES: C=CC(=O)N1CCN(c2nccnc2Oc2ccc(OC(F)F)cc2)CC1]

Following the general procedure in scheme 11, 4-(difluoromethoxy)phenol was used in step 1. Offered 1-(4-(3-(4-(difluoromethoxy)phenoxy)pyrazin-2-yl)piperazin-1-yl)prop-2-en-1-one as white solid. (Positive, ES, m/z): 377.13. ¹H NMR (400 MHz, DMSO) δ 7.95 (d, J=2.8 Hz, 1H), 7.58 (d, J=2.8 Hz, 1H), 7.48-7.03 (m, 5H), 6.86 (dd, J=16.7, 10.4 Hz, 1H), 6.16 (dd, J=16.7, 2.4 Hz, 1H), 5.73 (dd, J=10.4, 2.4 Hz, 1H), 3.83-3.64 (m, 4H), 3.58 (t, J=5.1 Hz, 4H).

### Example 72: Synthesis of 1-(4-(3-(4-(trifluoromethoxy)phenoxy)pyrazin-2-yl)piperazin-1-yl)prop-2-en-1-one

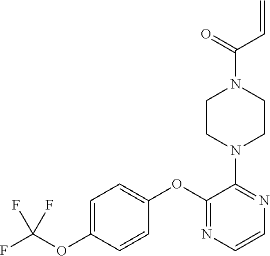 [SMILES: C=CC(=O)N1CCN(c2nccnc2Oc2ccc(OC(F)(F)F)cc2)CC1]

Following the general procedure in scheme 11, 4-(trifluoromethoxy)phenol was used in step 1. Offered 1-(4-(3-(4-(trifluoromethoxy)phenoxy)pyrazin-2-yl)piperazin-1-yl)prop-2-en-1-one as white solid. (Positive, ES, m/z): 395.27. ¹H NMR (400 MHz, DMSO) δ 7.97 (d, J=2.8 Hz, 1H), 7.59 (d, J=2.8 Hz, 1H), 7.50-7.39 (m, 2H), 7.41-7.29 (m, 2H), 6.85 (dd, J=16.8, 10.5 Hz, 1H), 6.16 (dd, J=16.7, 2.4 Hz, 1H), 5.73 (dd, J=10.4, 2.4 Hz, 1H), 3.72 (d, J=17.7 Hz, 4H), 3.57 (d, J=6.8 Hz, 4H).

### Example 73: Synthesis of 1-(4-(3-((5-(trifluoromethyl)pyridin-2-yl)oxy)pyrazin-2-yl)piperazin-1-yl)prop-2-en-1-one

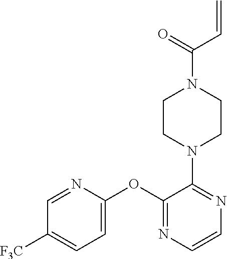 [SMILES: C=CC(=O)N1CCN(c2nccnc2Oc2ccc(C(F)(F)F)cn2)CC1]

Following the general procedure in scheme 11, 5-(trifluoromethyl)pyridin-2-ol was used in step 1. Offered 1-(4-(3-((5-(trifluoromethyl)pyridin-2-yl)oxy)pyrazin-2-yl)piperazin-1-yl)prop-2-en-1-one as white solid. (Positive, ES, m/z): 380.26. ¹H NMR (400 MHz, DMSO) δ 8.69 (d, J=3.4 Hz, 1H), 8.36 (dd, J=8.5, 3.1 Hz, 1H), 8.15 (q, J=2.5 Hz, 1H), 7.77 (q, J=2.5 Hz, 1H), 7.49 (dd, J=9.0, 3.3 Hz, 1H), 6.81 (dd, J=16.7, 10.6 Hz, 1H), 6.13 (dt, J=16.7, 2.2 Hz, 1H), 5.70 (dt, J=10.4, 2.3 Hz, 1H), 3.69-3.45 (m, 8H).

### Example 74: Synthesis of 1-(4-(3-((5-(trifluoromethyl)pyridin-2-yl)oxy)pyrazin-2-yl)-3,6-dihydropyridin-1(2H)-yl)prop-2-en-1-one

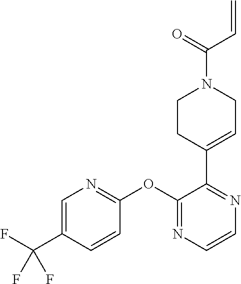 [SMILES: C=CC(=O)N1CC=C(c2nccnc2Oc2ccc(C(F)(F)F)cn2)CC1]

Following the general procedure in scheme 6, 2-chloro-3-((5-(trifluoromethyl)pyridin-2-yl)oxy)pyrazine was used in step 1. Offered 1-(4-(3-((5-(trifluoromethyl)pyridin-2-yl)oxy)pyrazin-2-yl)-3,6-dihydropyridin-1(2H)-yl)prop-2-en-1-one as white solid. (Positive, ES, m/z): 377.21. ¹H NMR (400 MHz, DMSO) δ 8.68 (d, I=2.6 Hz, 1H), 8.65-8.54 (m, 1H), 8.37 (dd, J=8.7, 2.6 Hz, 1H), 8.33-8.21 (m, 1H), 7.51 (d, J=8.6 Hz, 1H), 6.93-6.69 (m, 2H), 6.13 (dd, J=16.8, 2.4 Hz, 1H), 5.70 (dd, J=10.4, 2.4 Hz, 1H), 4.38-4.12 (m, 2H), 3.71 (dt, J=16.3, 5.7 Hz, 2H), 2.77-2.54 (m, 2H).

### Example 75: Synthesis of 1-(4-(3-((5-(trifluoromethyl)pyridin-2-yl)oxy)pyrazin-2-yl)piperidin-1-yl)prop-2-en-1-one

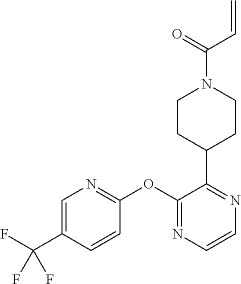 [SMILES: C=CC(=O)N1CCC(c2nccnc2Oc2ccc(C(F)(F)F)cn2)CC1]

Following the general procedure in scheme 7, tert-butyl 4-(3-((5-(trifluoromethyl)pyridin-2-yl)oxy)pyrazin-2-yl)-3,6-dihydropyridine-1(2H)-carboxylate was used in step 1. Offered 1-(4-(3-((5-(trifluoromethyl)pyridin-2-yl)oxy)pyrazin-2-yl)piperidin-1-yl)prop-2-en-1-one as white solid. (Positive, ES, m/z): 391.54. ¹H NMR (400 MHz, DMSO) δ 8.84 (d, 0.1=2.4 Hz, 1H), 8.67-8.55 (m, 2H), 7.92 (dd, 1=9.7, 2.8 Hz, 1H), 6.84 (td, I=10.7, 5.0 Hz, 1H), 6.75 (d, J=9.7 Hz, 1H), 6.12 (dd, J=16.7, 2.5 Hz, 1H), 5.68 (d, J=10.6 Hz, 1H), 4.60-4.40 (m, 1H), 4.26-4.02 (m, 1H), 3.09 (t, J=13.0 Hz, 1H), 2.93-2.79 (m, 1H), 2.76-2.61 (m, 1H), 1.89 (s, 1H), 1.74 (t, J=14.4 Hz, 2H), 1.59 (d, J=12.9 Hz, 1H).

### Example 76: Synthesis of 2-fluoro-1-(4-(3-((5-(trifluoromethyl)pyridin-2-yl)oxy)pyrazin-2-yl)piperidin-1-yl)prop-2-en-1-one

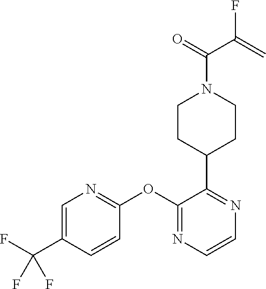 [SMILES: C=C(F)C(=O)N1CCC(c2nccnc2Oc2ccc(C(F)(F)F)cn2)CC1]

Following the general procedure in scheme 10, 2-(piperidin-4-yl)-3-((5-(trifluoromethyl)pyridin-2-yl)oxy)pyrazine was used. Offered 2-fluoro-1-(4-(3-((5-(trifluoromethyl)pyridin-2-yl)oxy)pyrazin-2-yl)piperidin-1-yl)prop-2-en-1-one as white solid. (Positive, ES, m/z): 397.03. ¹H NMR (400 MHz, DMSO) δ 8.70 (s, 1H), 8.53 (d, J=2.6 Hz, 1H), 8.38 (d, J=8.9 Hz, 1H), 8.26 (d, J=2.6 Hz, 1H), 7.52 (d, J=8.7 Hz, 1H), 5.33-5.08 (m, 2H), 3.31 (s, 2H), 1.94 (d, J=13.3 Hz, 2H), 1.75 (s, 2H).

### Example 77: Synthesis of 1-(4-(3-((6-(trifluoromethyl)pyridin-3-yl)oxy)pyrazin-2-yl)piperazin-1-yl)prop-2-en-1-one

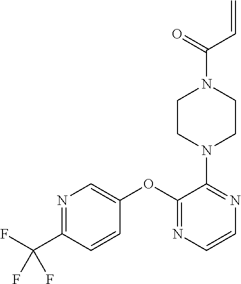 [SMILES: C=CC(=O)N1CCN(c2nccnc2Oc2ccc(C(F)(F)F)nc2)CC1]

Following the general procedure in scheme 11, 6-(trifluoromethyl)pyridin-3-ol was used in step 1. Offered 1-(4-(3-((6-(trifluoromethyl)pyridin-3-yl)oxy)pyrazin-2-yl)piperazin-1-yl)prop-2-en-1-one as white solid. (Positive, ES, m/z): 380.22. ¹H NMR (400 MHz, DMSO) δ 8.77 (t, J=1.7 Hz, 1H), 8.04 (t, J=2.3 Hz, 3H), 7.61 (d, J=2.7 Hz, 1H), 6.86 (dd, J=16.7, 10.4 Hz, 1H), 6.16 (dd, J=16.7, 2.4 Hz, 1H), 5.73 (dd, J=10.4, 2.4 Hz, 1H), 3.73 (dd, J=17.9, 5.7 Hz, 4H), 3.61 (d, J=6.1 Hz, 4H).

### Example 78: Synthesis of 2-fluoro-1-(4-(3-((6-(trifluoromethyl)pyridin-3-yl)oxy)pyrazin-2-yl)piperazin-1-yl)prop-2-en-1-one

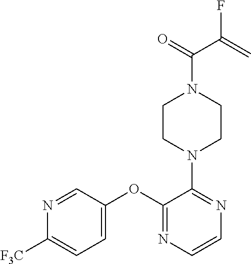 [SMILES: C=C(F)C(=O)N1CCN(c2nccnc2Oc2ccc(C(F)(F)F)nc2)CC1]

Following the general procedure in scheme 10, 2-(piperazin-1-yl)-3-((6-(trifluoromethyl)pyridin-3-yl)oxy)pyrazine was used. Offered 2-fluoro-1-(4-(3-((6-(trifluoromethyl)pyridin-3-yl)oxy)pyrazin-2-yl)piperazin-1-yl)prop-2-en-1-one as white solid. (Positive, ES, m/z): 398.17. ¹H NMR (400 MHz, DMSO) δ 8.77 (s, 1H), 8.04 (d, J=2.1 Hz, 3H), 7.62 (t, J=2.3 Hz, 1H), 5.39-5.15 (m, 2H), 3.77-3.63 (m, 8H).

### Example 79: Synthesis of 1-(4-(3-((6-(trifluoromethyl)pyridin-3-yl)oxy)pyrazin-2-yl)piperidin-1-yl)prop-2-en-1-one

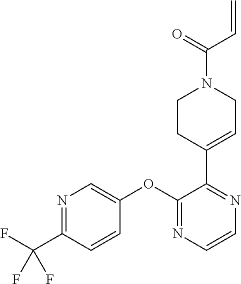 [SMILES: C=CC(=O)N1CC=C(c2nccnc2Oc2ccc(C(F)(F)F)nc2)CC1]

Following the general procedure in scheme 7, tert-butyl 4-(3-((6-(trifluoromethyl)pyridin-3-yl)oxy)pyrazin-2-yl)-3,6-dihydropyridine-1(2H)-carboxylate was used in step 1. Offered 1-(4-(3-((6-(trifluoromethyl)pyridin-3-yl)oxy)pyrazin-2-yl)piperidin-1-yl)prop-2-en-1-one as white solid. (Positive, ES, m/z): 379.23. ¹H NMR (400 MHz, DMSO) δ 8.78 (d, J=1.7 Hz, 1H), 8.41 (d, J=2.6 Hz, 1H), 8.07 (dd, J=9.7, 2.2 Hz, 3H), 6.87 (dd, J=16.7, 10.4 Hz, 1H), 6.13 (dd, J=16.7, 2.4 Hz, 1H), 5.70 (dd, J=10.4, 2.5 Hz, 1H), 4.58 (d, J=13.0 Hz, 1H), 4.22 (d, J=13.6 Hz, 1H), 3.51 (tt, J=11.6, 3.7 Hz, 1H), 3.28 (t, J=13.0 Hz, 1H), 2.86 (t, J=12.5 Hz, 1H), 2.11-1.94 (m, 2H), 1.86-1.57 (m, 2H).

### Example 80: Synthesis of 2-fluoro-1-(4-(3-((6-(trifluoromethyl)pyridin-3-yl)oxy)pyrazin-2-yl)piperidin-1-yl)prop-2-en-1-one

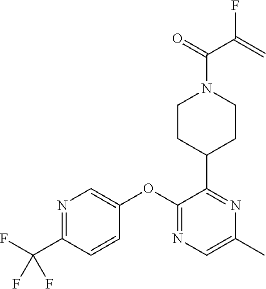 [SMILES: C=C(F)C(=O)N1CCC(c2nc(C)cnc2Oc2ccc(C(F)(F)F)nc2)CC1]

Following the general procedure in scheme 10, 2-(piperidin-4-yl)-3-((6-(trifluoromethyl)pyridin-3-yl)oxy)pyrazine was used. Offered 2-fluoro-1-(4-(3-((6-(trifluoromethyl)pyridin-3-yl)oxy)pyrazin-2-yl)piperidin-1-yl)prop-2-en-1-one as white solid. (Positive, ES, m/z): 447.1. ¹H NMR (400 MHz, DMSO) δ 8.78 (d, J=1.8 Hz, 1H), 8.42 (d, J=2.7 Hz, 1H), 8.07 (dd, J=13.1, 2.2 Hz, 3H), 6.24 (s, 1H), 6.02 (s, 1H), 4.53 (d, J=13.0 Hz, 1H), 3.96 (d, J=13.5 Hz, 1H), 3.54 (t, J=11.6 Hz, 1H), 3.00 (d, J=12.9 Hz, 1H), 2.17-1.97 (m, 2H), 1.90-1.53 (m, 2H).

### Example 81: Synthesis of 2-(trifluoromethyl)-1-(4-(3-((6-(trifluoromethyl)pyridin-3-yl)oxy)pyrazin-2-yl)piperidin-1-yl)prop-2-en-1-one

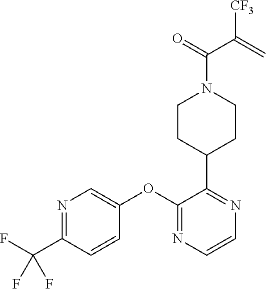 [SMILES: C=C(C(=O)N1CCC(c2nccnc2Oc2ccc(C(F)(F)F)nc2)CC1)C(F)(F)F]

Following the general procedure in scheme 10, 2-(piperidin-4-yl)-3-((6-(trifluoromethyl)pyridin-3-yl)oxy)pyrazine and 2-(trifluoromethyl)acrylic acid was used. Offered 2-(trifluoromethyl)-1-(4-(3-((6-(trifluoromethyl)pyridin-3-yl)oxy)pyrazin-2-yl)piperidin-1-yl)prop-2-en-1-one as white solid. (Positive, ES, m/z): 447.1. ¹H NMR (400 MHz, DMSO) δ 8.78 (s, 1H), 8.42 (d, J=2.6 Hz, 1H), 8.15-7.97 (m, 3H), 6.24 (s, 1H), 6.02 (s, 1H), 4.53 (d, J=13.0 Hz, 1H), 3.95 (d, J=13.6 Hz, 1H), 3.54 (t, J=11.4 Hz, 1H), 3.33 (s, 1H), 2.98 (t, J=13.1 Hz, 1H), 2.05 (t, J=17.8 Hz, 2H), 1.88-1.65 (m, 2H).

### Example 82. Synthesis of 2-((4-(3-((6-(trifluoromethyl)pyridin-3-yl)oxy)pyrazin-2-yl)piperidin-1-yl)methyl)acrylic acid

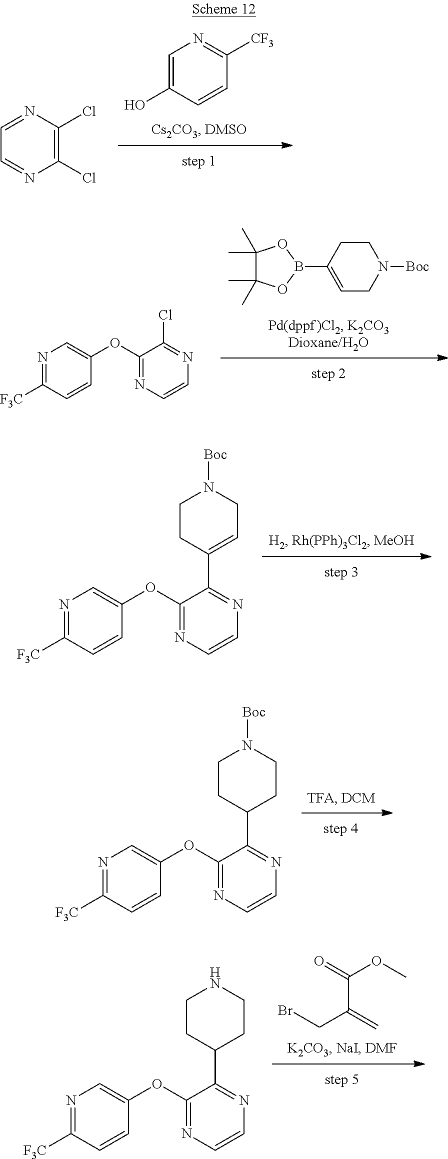

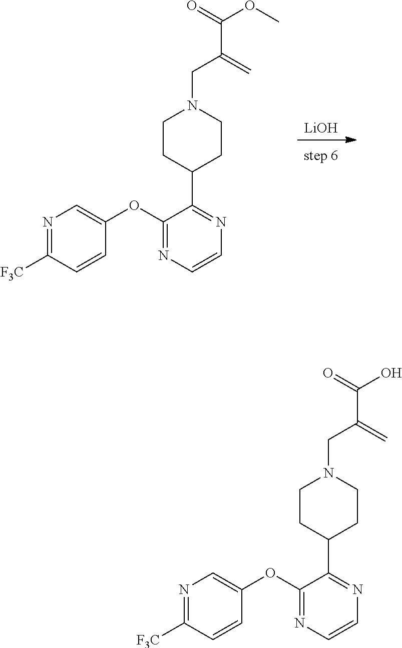 [SMILES: C=C(CN1CCC(c2nccnc2Oc2ccc(C(F)(F)F)nc2)CC1)C(=O)OC ; "LiOH" ; C=C(CN1CCC(c2nccnc2Oc2ccc(C(F)(F)F)nc2)CC1)C(=O)O]

### Step 1: Synthesis of 2-chloro-3-{[6-(trifluoromethyl) pyridin-3-yl]oxy}pyrazine

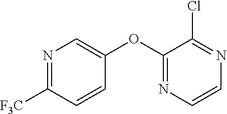 [SMILES: FC(F)(F)c1ccc(Oc2nccnc2Cl)cn1]

To a solution of 2,3-dichloropyrazine (18 g, 120.830 mmol, 1 equiv), 6-(trifluoromethyl) pyridin-3-ol (15.77 g, 96.664 mmol, 0.8 equiv) and in DMSO (200 mL) was added Cs₂CO₃ (59.05 g, 181.245 mmol, 1.5 equiv), then stirred for 2 h at room temperature. The reaction mixture was quenched with water (200 mL) and extracted with EtOAc (3×300 mL). The organic layer was combined and washed with saturated brine (2×300 mL), dried over anhydrous Na₂SO₄. After filtration, the filtrate was concentrated under reduced pressure. The residue was purified by silica gel column chromatography, eluted with PE/EA (3:1). This resulted in 2-chloro-3-{[6-(trifluoromethyl) pyridin-3-yl]oxy}pyrazine (21 g, 63.06%) as a yellow solid. LCMS (Positive, ES, m/z): 276.1

### Step 2: Synthesis of tert-butyl 4-(3-{[6-(trifluoromethyl)pyridin-3-yl]oxy}pyrazin-2-yl)-3,6-dihydro-2H-pyridine-1-carboxylate

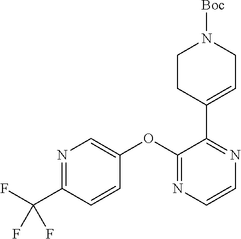 [SMILES: CC(C)(C)OC(=O)N1CC=C(c2nccnc2Oc2ccc(C(F)(F)F)nc2)CC1]

To a solution of 2-chloro-3-{[6-(trifluoromethyl) pyridin-3-yl]oxy}pyrazine (1.1 g, 3.991 mmol, 1 equiv) and tert-butyl 4-(4,4,5,5-tetramethyl-1,3,2-dioxaborolan-2-yl)-3,6-dihydro-2H-pyridine-1-carboxylate (1.48 g, 4.789 mmol, 1.2 equiv) in dioxane/H₂O (10 mL/1 mL) were added K₂CO₃ (1.65 g, 11.973 mmol, 3 equiv) and Pd(dppf)Cl₂.CH₂Cl₂ (0.33 g, 0.399 mmol, 0.1 equiv). After stirring for 5 h at 100° C. under a nitrogen atmosphere, the resulting mixture was concentrated under reduced pressure. The residue was purified by silica gel column chromatography, eluted with PE/THF (3:1). This resulted in tert-butyl 4-(3-{[6-(trifluoromethyl)pyridin-3-yl]oxy}pyrazin-2-yl)-3,6-dihydro-2H-pyridine-1-carboxylate (1.65 g, 97.87%) as a yellow solid. LCMS (Positive, ES, m/z): 423.2

### Step 3: Synthesis oftert-butyl-4-(3-{[6-(trifluoromethyl) pyridin-3-yl]oxy}pyrazin-2-yl) piperidine-1-carboxylate

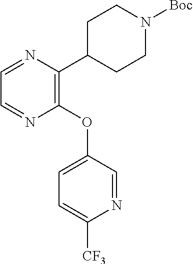 [SMILES: CC(C)(C)OC(=O)N1CCC(c2nccnc2Oc2ccc(C(F)(F)F)nc2)CC1]

A solution of tert-butyl 4-(3-{[6-(trifluoromethyl) pyridin-3-yl]oxy}pyrazin-2-yl)-3,6-dihydro-2H-pyridine-1-carboxylate (800 mg, 1.894 mmol, 1 equiv) and Rh (PPh₃)₃Cl₂ (181.60 mg, 0.189 mmol, 0.1 equiv) in MeOH (6 ml) and THF (3 ml) was stirred for overnight at room temperature under hydrogen atmosphere (10 atm). The mixture was concentrated under reduced pressure and purified by silica gel column chromatography, eluted with PE/EA (1:1). This resulted in tert-butyl-4-(3-{[6-(trifluoromethyl) pyridin-3-yl]oxy}pyrazin-2-yl) piperidine-1-carboxylate (720 mg, 89.44%) as a white solid. LCMS (Positive, ES, m/z): 425.0.

### Step 4: Synthesis of 2-(piperidin-4-yl)-3-{[6-(trifluoromethyl) pyridin-3-yl]oxy}pyrazine

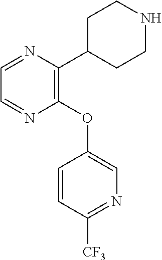 [SMILES: FC(F)(F)c1ccc(Oc2nccnc2C2CCNCC2)cn1]

A mixture of tert-butyl 4-(3-{[6-(trifluoromethyl) pyridin-3-yl]oxy}pyrazin-2-yl) piperidine-1-carboxylate (5 g, 11.781 mmol, 1 equiv) in DCM (40 mL) and TFA (10 mL) was stirred for 2 h at room temperature. The resulting mixture was concentrated under reduced pressure to give 2-(piperidin-4-yl)-3-{[6-(trifluoromethyl) pyridin-3-yl]oxy}pyrazine) (3.6 g, 94.03%) as a white solid. The crude was used in the next step directly without further purification. LCMS-PH (Positive, ES, m/z): 325.3.

### Step 5: Synthesis of methyl 2-{[4-(3-{[6-(trifluoromethyl) pyridin-3-yl]oxy}pyrazin-2-yl) piperidin-1-yl]methyl}prop-2-enoate

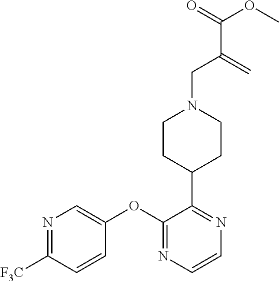 [SMILES: C=C(CN1CCC(c2nccnc2Oc2ccc(C(F)(F)F)nc2)CC1)C(=O)OC]

To a solution of 2-(piperidin-4-yl)-3-{[6-(trifluoromethyl) pyridin-3-yl]oxy}pyrazine (500 mg, 1.542 mmol, 1 equiv) and methyl 2-(bromomethyl) prop-2-enoate (331.19 mg, 1.850 mmol, 1.2 equiv) in MeCN (10 mL) was added NaI (277.32 mg, 1.850 mmol, 1.2 equiv) and K₂CO₃ (639.23 mg, 4.626 mmol, 3 equiv), then stirred for 2 h at 60° C. The reaction mixture was quenched with water (20 mL) and extracted with EtOAc (3×20 mL). The organic layer was combined and washed with saturated brine (2×20 mL), dried over anhydrous Na₂SO₄. After filtration, the filtrate was concentrated under reduced pressure and resulted in methyl 2-{[4-(3-{[6-(trifluoromethyl) pyridin-3-yl]oxy}pyrazin-2-yl) piperidin-1-yl]methyl}prop-2-enoate (647 mg, 72.82%) as an orange oil. The crude product was used in the next step directly without further purification.

### Step 6: Synthesis of 2-((4-(3-((6-(trifluoromethyl)pyridin-3-yl)oxy)pyrazin-2-yl)piperidin-1-yl)methyl)acrylic acid

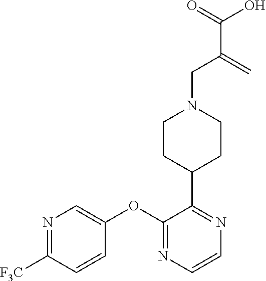 [SMILES: C=C(CN1CCC(c2nccnc2Oc2ccc(C(F)(F)F)nc2)CC1)C(=O)O]

To a solution of methyl 2-{[4-(3-{[6-(trifluoromethyl) pyridin-3-yl]oxy}pyrazin-²-yl) piperidin-1-yl]methyl}prop-2-enoate (600 mg, 1.420 mmol, 1 equiv) in THF/H₂O (8 mL, 3:1) was added LiOH—H₂O (238.40 mg, 5.680 mmol, 4 equiv) and stirred for 12 h at room temperature. The mixture was acidified to pH 6 with iN HCl (aq.) and extracted with EtOAc (3×30 mL). The organic layer was combined and washed with saturated brine (2×30 mL), dried over anhydrous Na₂SO₄ and concentrated. The crude product was purified by (Column: Welch Xtimate C18 ExRS, 250 mm, l0 μm; Mobile Phase A: Water (0.05% NH₃·H₂O), Mobile Phase B: ACN; Flow rate: 90 mL/min; Gradient: 90% B to 90% B in 10 min, 90% B; Wave Length: 254 nm; RT1(min): 7; Number Of Runs: 5; to afford 2-{[4-(3-{[6-(trifluoromethyl)pyridin-3-yl]oxy}pyrazin-2-yl)piperidin-1-yl]methyl}prop-2-enoic acid (330 mg, 56.89%) as a white solid. LCMS (Positive, ES, m/z): 409.2. ¹H NMR (300 MHz, DMSO-d₆) δ 8.75 (d, J=2.0 Hz, 1H), 8.41 (d, J=2.7 Hz, 1H), 8.05 (d, J=2.7 Hz, 1H), 8.03 (d, J=1.7 Hz, 2H), 5.88 (d, J=3.2 Hz, 1H), 5.38 (d, J=2.9 Hz, 1H), 3.20 (s, 3H), 3.03 (d, J=11.2 Hz, 2H), 2.18 (dd, J=12.1, 8.5 Hz, 2H), 1.97-1.80 (m, 4H).

### Example 83. Synthesis of 1-(4-hydroxy-4-(3-((6-(trifluoromethyl)pyridin-3-yl)oxy)pyrazin-2-yl)piperidin-1-yl)prop-2-en-1-one

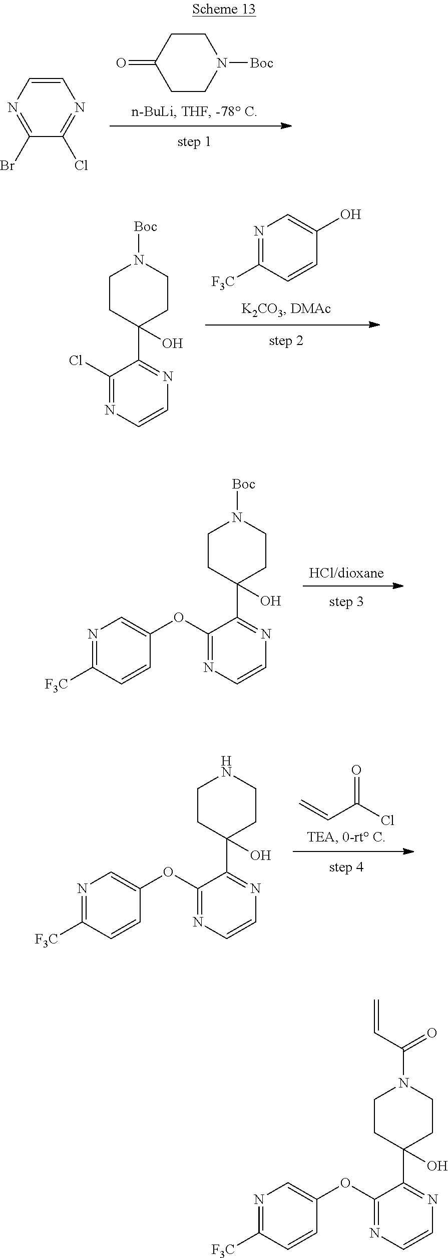

### Step 1: Synthesis of tert-butyl 4-(3-chloropyrazin-2-yl)-4-hydroxypiperidine-1-carboxylate

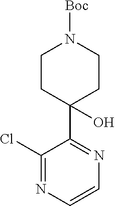 [SMILES: CC(C)(C)OC(=O)N1CCC(O)(c2nccnc2Cl)CC1]

To a stirred solution of 2-bromo-3-chloropyrazine (1.6 g, 8.27 mmol, 1 equiv) in THF (20 mL) was added n-BuLi (1.06 g, 16.54 mmol, 2 equiv) dropwise at −78° C. under nitrogen atmosphere. The resulting mixture was stirred for 1 h at −78° C. To the above mixture was added a solution of tert-butyl 4-oxopiperidine-1-carboxylate (1.81 g, 9.10 mmol, 1.1 equiv) in THF (10 mL) dropwise over 10 min at −78° C. The resulting mixture was stirred for additional 1 h at −78° C. After the reaction was completed, the reaction was quenched with H₂O (20 ml). The resulting mixture was extracted with EtOAc (3×10 mL). The combined organic layers were washed with brine (2×10 mL), dried over anhydrous Na₂SO₄. After filtration, the filtrate was concentrated under reduced pressure. The residue was purified by silica gel column chromatography, eluted with PE/EA (1:1) to afford tert-butyl 4-(3-chloropyrazin-2-yl)-4-hydroxypiperidine-1-carboxylate (1.3 g, 50.09%) as a yellow oil. LCMS:(ES, m/z):[M+H]⁺=314

### Step 2: Synthesis of tert-butyl 4-hydroxy-4-(3-{[6-(trifluoromethyl) pyridin-3-yl]oxy}pyrazin-2-yl) piperidine-1-carboxylate

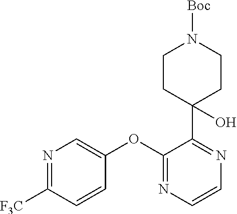 [SMILES: CC(C)(C)OC(=O)N1CCC(O)(c2nccnc2Oc2ccc(C(F)(F)F)nc2)CC1]

To a stirred solution of tert-butyl 4-(3-chloropyrazin-2-yl)-4-hydroxypiperidine-1-carboxylate (1.3 g, 4.14 mmol, 1 equiv) and 6-(trifluoromethyl) pyridin-3-ol (743.30 mg, 4.56 mmol, 1.1 equiv) in DMAc (15 mL, 161.32 mmol,38.94equiv) was added K₂CO₃ (1145.17 mg, 8.29 mmol, 2.0 equiv) at rt. The resulting mixture was stirred for overnight at 110° C. The reaction was quenched by the addition of water (100 mL) at room temperature. The resulting mixture was extracted with EtOAc (3×50 mL). The combined organic layers were washed with brine (2×50 mL), dried over anhydrous Na₂SO₄. After filtration, the filtrate was concentrated under reduced pressure. The residue was purified by reversed-phase flash chromatography with the following conditions: column, C18 silica gel; mobile phase, ACN in Water (0.1% FA), 10% to 40% gradient in 10 min; detector, UV 254 nm. This resulted in tert-butyl 4-hydroxy-4-(3-{[6-(trifluoromethyl) pyridin-3-yl]oxy}pyrazin-2-yl) piperidine-1-carboxylate (250 mg, 13.70%) as a white solid. LCMS (ES, m/z):[M+H]⁺=441.

### Step 3: Synthesis of 4-(3-{[6-(trifluoromethyl) pyridin-3-yl]oxy}pyrazin-2-yl) piperidin-4-ol

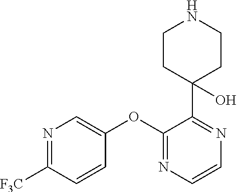 [SMILES: OC1(c2nccnc2Oc2ccc(C(F)(F)F)nc2)CCNCC1]

To a stirred solution of tert-butyl 4-hydroxy-4-(3-{[6-(trifluoromethyl) pyridin-3-yl]oxy}pyrazin-2-yl) piperidine-1-carboxylate (250 mg, 0.568 mmol, 1 equiv) in DCM (3 mL) was added HCl (gas)in 1,4-dioxane (0.50 mL, 16.46 mmol, 29.0 equiv) dropwise at 0° C. The resulting mixture was stirred for 1 h at 0° C. The resulting mixture was concentrated under reduced pressure. The residue was purified by reversed-phase flash chromatography with the following conditions: column, C18 silica gel; mobile phase, ACN in Water (10 mmol/L NH₄HCO₃), 5% to 25% gradient in 10 min; detector, UV 254 nm. This resulted in 4-(3-{[6-(trifluoromethyl) pyridin-3-yl]oxy}pyrazin-2-yl) piperidin-4-ol (120 mg, 62.12%) as a yellow solid. LCMS (ES, m/z):[M+H]⁺=341.

### Step 4: Synthesis of 1-(4-hydroxy-4-(3-((6-(trifluoromethyl)pyridin-3-yl)oxy)pyrazin-2-yl)piperidin-1-yl)prop-2-en-1-one

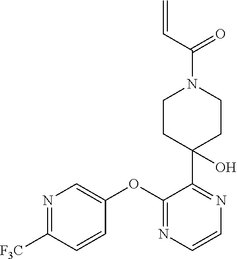 [SMILES: C=CC(=O)N1CCC(O)(c2nccnc2Oc2ccc(C(F)(F)F)nc2)CC1]

A solution of 4-(3-{[6-(trifluoromethyl) pyridin-3-yl]oxy}pyrazin-2-yl) piperidin-4-ol (120 mg, 0.353 mmol, 1 equiv) and TEA (71.37 mg, 0.70 mmol, 2 equiv) in DCM (5 mL) was stirred at 0° C. followed by the addition of acryloyl chloride (63.83 mg, 0.70 mmol, 2 equiv) dropwise at 0° C. The resulting mixture was stirred for 1 h at rt. The reaction was quenched with water/ice(5 mL) at 0° C. The resulting mixture was extracted with DCM (3×10 mL). The combined organic layers were washed with brine (1×10 mL), dried over anhydrous Na₂SO₄. After filtration, the filtrate was concentrated under reduced pressure. The residue was purified by Prep-HPLC with the following conditions (Column: X-Bridge Prep OBD C18 Column, 30*150 mm, 5 m; Mobile Phase A: Water 0.1% NH3. H₂O), Mobile Phase B: ACN; Flow rate: 60 mL/min; Gradient: 16% B to 41% B in 8 min, 41% B; Wave Length: 220 nm; RT (min): 7.56 to afford 1-(4-hydroxy-4-(3-((6-(trifluoromethyl)pyridin-3-yl)oxy)pyrazin-2-yl)piperidin-1-yl)prop-2-en-1-one as a white solid (4 mg, 2.88%). LCMS (ES, m/z): [M+H]-=395. ¹H NMR (400 MHz, DMSO-d₆) δ 8.68 (d, J=2.6 Hz, 1H), 8.43 (d, J=2.6 Hz, 1H), 8.18 (d, J=2.6 Hz, 1H), 8.02 (d, J=8.6 Hz, 1H), 7.93 (dd, J=8.5, 2.6 Hz, 1H), 6.85 (dd, J=16.7, 10.5 Hz, 1H), 6.09 (dd, J=16.7, 2.5 Hz, 1H), 5.66 (dd, J=10.4, 2.5 Hz, 1H), 5.53 (s, 1H), 4.25 (m, 1H), 3.95 (m, 1H), 3.57 (m, 1H), 3.22 (m, 1H), 2.35-2.05 (m, 4H).

### Example 84: Synthesis of 1-(4-(3-((6-(trifluoromethyl)pyridin-3-yl)oxy)pyrazin-2-yl)-3,6-dihydropyridin-1(2H)-yl)prop-2-en-1-one

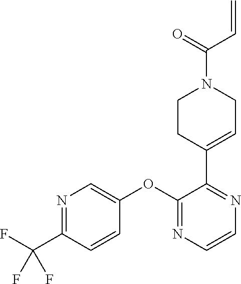 [SMILES: C=CC(=O)N1CC=C(c2nccnc2Oc2ccc(C(F)(F)F)nc2)CC1]

Following the general procedure in scheme 6, 2-chloro-3-((6-(trifluoromethyl)pyridin-3-yl)oxy)pyrazine was used in step 1. Offered 1-(4-(3-((6-(trifluoromethyl)pyridin-3-yl)oxy)pyrazin-2-yl)-3,6-dihydropyridin-1(2H)-yl)prop-2-en-1-one as white solid. (Positive, ES, m/z): 377.21. ¹H NMR (400 MHz, DMSO) δ 8.78 (t, J=1.6 Hz, 1H), 8.47 (t, J=2.5 Hz, 1H), 8.13-7.97 (m, 3H), 7.06-6.76 (m, 2H), 6.17 (dt, J=16.8, 3.3 Hz, 1H), 5.73 (dd, J=10.4, 2.4 Hz, 1H), 4.47-4.25 (m, 2H), 3.80 (dt, 1=14.1, 5.6 Hz, 2H), 2.83-2.63 (m, 2H).

### Example 85: Synthesis of 2-fluoro-1-(4-(3-((6-(trifluoromethyl)pyridin-3-yl)oxy)pyrazin-2-yl)-3,6-dihydropyridin-1(2H)-yl)prop-2-en-1-one

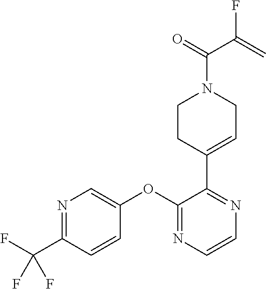 [SMILES: C=C(F)C(=O)N1CC=C(c2nccnc2Oc2ccc(C(F)(F)F)nc2)CC1]

Following the general procedure in scheme 10, 2-(1,2,3,6-tetrahydropyridin-4-yl)-3-((6-(trifluoromethyl)pyridin-3-yl)oxy)pyrazine was used. Offered 2-fluoro-1-(4-(3-((6-(trifluoromethyl)pyridin-3-yl)oxy)pyrazin-2-yl)-3,6-dihydropyridin-1(2H)-yl)prop-2-en-1-one as white solid. (Positive, ES, m/z): 395.31. ¹H NMR (400 MHz, DMSO) δ 8.78 (s, 1H), 8.47 (d, J=2.5 Hz, 1H), 8.07 (d, J=16.8 Hz, 3H), 7.02 (s, 1H), 5.42-5.15 (m, 2H), 4.44-4.17 (m, 2H), 3.78 (t, J=5.6 Hz, 2H), 2.79 (s, 2H).

### Example 86: Synthesis of 2-((3-(3-((6-(trifluoromethyl)pyridin-3-yl)oxy)pyrazin-2-yl)azetidin-1-yl)methyl)acrylic acid

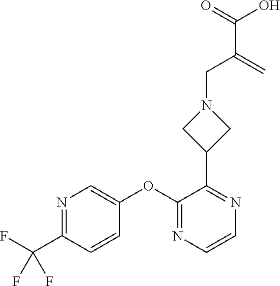 [SMILES: C=C(CN1CC(c2nccnc2Oc2ccc(C(F)(F)F)nc2)C1)C(=O)O]

Following the general procedure in scheme 12, 2-(azetidin-3-yl)-3-((6-(trifluoromethyl)pyridin-3-yl)oxy)pyrazine was used in step 5. Offered 2-((3-(3-((6-(trifluoromethyl)pyridin-3-yl)oxy)pyrazin-2-yl)azetidin-1-yl)methyl)acrylic acid as white solid. (Positive, ES, m/z): 381.1. ¹H NMR (400 MHz, DMSO) δ 8.74 (t, J=1.7 Hz, 1H), 8.44 (d, J=2.7 Hz, 1H), 8.11-7.95 (m, 3H), 5.70 (d, =3.7 Hz, 1H), 5.15 (d, =3.6 Hz, 1H), 4.10 (t, J=7.8 Hz, 1H), 3.70 (t, J=7.4 Hz, 2H), 3.21 (s, 2H).

### Example 87: Synthesis of 1-(4-(3-((2-(trifluoromethyl)pyrimidin-5-yl)oxy)pyrazin-2-yl)piperazin-1-yl)prop-2-en-1-one

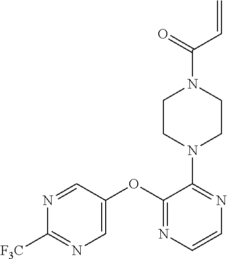 [SMILES: C=CC(=O)N1CCN(c2nccnc2Oc2cnc(C(F)(F)F)nc2)CC1]

Following the general procedure in scheme 11, 2-(trifluoromethyl)pyrimidin-5-ol was used in step 1. Offered 1-(4-(3-((2-(trifluoromethyl)pyrimidin-5-yl)oxy)pyrazin-2-yl)piperazin-1-yl)prop-2-en-1-one as white solid. (Positive, ES, m/z): 381.02. ¹H NMR (400 MHz, DMSO) δ 9.18 (s, 2H), 8.08 (d, J=2.7 Hz, 1H), 7.63 (d, J=2.7 Hz, 1H), 6.87 (dd, J=16.7, 10.4 Hz, 1H), 6.17 (dd, J=16.7, 2.4 Hz, 1H), 5.74 (dd, J=10.4, 2.4 Hz, 1H), 3.83-3.67 (m, 4H), 3.63 (dd, J=6.7, 3.5 Hz, 4H).

### Example 88: Synthesis of 2-fluoro-1-(4-(3-((2-(trifluoromethyl)pyrimidin-5-yl)oxy)pyrazin-2-yl)piperazin-1-yl)prop-2-en-1-one

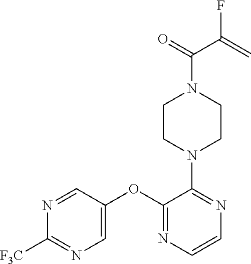 [SMILES: C=C(F)C(=O)N1CCN(c2nccnc2Oc2cnc(C(F)(F)F)nc2)CC1]

Following the general procedure in scheme 10, 5-((3-(piperazin-1-yl)pyrazin-2-yl)oxy)-2-(trifluoromethyl)pyrimidine was used. Offered 2-fluoro-1-(4-(3-((2-(trifluoromethyl)pyrimidin-5-yl)oxy)pyrazin-2-yl)piperazin-1-yl)prop-2-en-1-one as white solid. (Positive, ES, m/z): 399.08. ¹H NMR (400 MHz, DMSO) δ 9.17 (s, 2H), 8.08 (d, J=2.3 Hz, 1H), 7.64 (d, J=2.4 Hz, 1H), 5.40-5.16 (m, 2H), 3.77-3.65 (m, 8H).

### Example 89: Synthesis of 1-(4-(3-((2-(trifluoromethyl)pyrimidin-5-yl)oxy)pyrazin-2-yl)-3,6-dihydropyridin-1(2H)-yl)prop-2-en-1-one

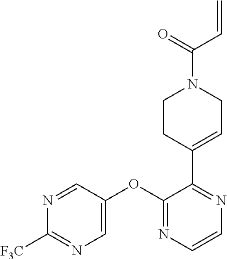 [SMILES: C=CC(=O)N1CC=C(c2nccnc2Oc2cnc(C(F)(F)F)nc2)CC1]

Following the general procedure in scheme 6, 5-((3-chloropyrazin-2-yl)oxy)-2-(trifluoromethyl)pyrimidine was used in step 1. Offered 1-(4-(3-((2-(trifluoromethyl)pyrimidin-5-yl)oxy)pyrazin-2-yl)-3,6-dihydropyridin-1(2H)-yl)prop-2-en-1-one as white solid. (Positive, ES, m/z): 378.3. ¹H NMR (400 MHz, DMSO) δ 9.19 (s, 2H), 8.52 (d, J=2.5 Hz, 1H), 8.11 (s, 1H), 7.09-6.76 (m, 2H), 6.17 (d, J=17.0 Hz, 1H), 5.74 (dd, J=10.5, 2.4 Hz, 1H), 4.48-4.24 (m, 2H), 3.82 (dd, J=13.7, 7.0 Hz, 2H), 2.79-2.70 (m, 2H).

### Example 90: Synthesis of 2-fluoro-1-(4-(3-((2-(trifluoromethyl)pyrimidin-5-yl)oxy)pyrazin-2-yl)-3,6-dihydropyridin-1(2H)-yl)prop-2-en-1-one

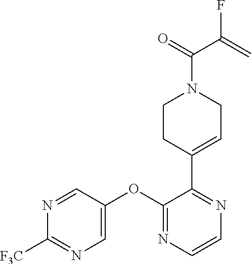 [SMILES: C=C(F)C(=O)N1CC=C(c2nccnc2Oc2cnc(C(F)(F)F)nc2)CC1]

Following the general procedure in scheme 10, 5-((3-(1,2,3,6-tetrahydropyridin-4-yl)pyrazin-2-yl)oxy)-2-(trifluoromethyl)pyrimidine was used. Offered 2-fluoro-1-(4-(3-((2-(trifluoromethyl)pyrimidin-5-yl)oxy)pyrazin-2-yl)-3,6-dihydropyridin-1(2H)-yl)prop-2-en-1-one as white solid. (Positive, ES, m/z): 396.1. ¹H NMR (400 MHz, DMSO) δ 9.18 (d, J=8.1 Hz, 2H), 8.52 (d, J=7.9 Hz, 1H), 8.12 (d, J=7.4 Hz, 1H), 7.03 (s, 1H), 5.42-5.14 (m, 2H), 4.47-4.18 (m, 2H), 3.78 (d, J=7.7 Hz, 2H), 2.78 (s, 2H).

### Example 91: Synthesis of 1-(4-(3-((2-(trifluoromethyl)pyrimidin-5-yl)oxy)p_yrazin-2-yl)piperidin--1)pIrop-2-en-1-one

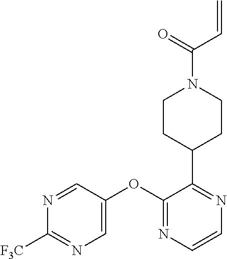 [SMILES: C=CC(=O)N1CCC(c2nccnc2Oc2cnc(C(F)(F)F)nc2)CC1]

Following the general procedure in scheme 7, tert-butyl 4-(3-((2-(trifluoromethyl)pyrimidin-5-yl)oxy)pyrazin-2-yl)-3,6-dihydropyridine-1(2H)-carboxylate was used in step 1. Offered 1-(4-(3-((2-(trifluoromethyl)pyrimidin-5-yl)oxy)pyrazin-2-yl)piperidin-1-yl)prop-2-en-1-one as white solid. (Positive, ES, m/z): 380.59. ¹H NMR (400 MHz, DMSO) δ 9.18 (s, 2H), 8.45 (d, J=2.7 Hz, 1H), 8.11 (d, J=2.7 Hz, 1H), 6.88 (dd, J=16.7, 10.5 Hz, 1H), 6.13 (dd, J=16.7, 2.4 Hz, 1H), 5.70 (dd, J=10.4, 2.5 Hz, 1H), 4.59 (d, J=13.1 Hz, 1H), 4.22 (d, J=13.7 Hz, 1H), 3.60-3.46 (m, 1H), 3.23 (d, J=10.9 Hz, 1H), 2.86 (t, J=12.8 Hz, 1H), 2.03 (d, J=13.6 Hz, 2H), 1.86-1.56 (m, 2H).

### Example 92: Synthesis of 2-fluoro-1-(4-(3-((2-(trifluoromethyl)pyrimidin-5-yl)oxy)pyrazin-2-yl)piperidin-1-yl)prop-2-en-1-one

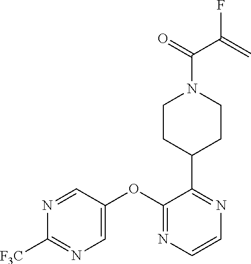 [SMILES: C=C(F)C(=O)N1CCC(c2nccnc2Oc2cnc(C(F)(F)F)nc2)CC1]

Following the general procedure in scheme 10, 5-((3-(piperidin-4-yl)pyrazin-2-yl)oxy)-2-(trifluoromethyl)pyrimidine was used. Offered 2-fluoro-1-(4-(3-((2-(trifluoromethyl)pyrimidin-5-yl)oxy)pyrazin-2-yl)piperidin-1-yl)prop-2-en-1-one as white solid. (Positive, ES, m/z): 398.05. ¹H NMR (400 MHz, DMSO) δ 9.18 (s, 2H), 8.46 (d, J=2.7 Hz, 1H), 8.11 (d, J=2.7 Hz, 1H), 5.39-5.08 (m, 2H), 4.43 (s, 1H), 4.06 (s, 1H), 3.56 (tt, J=11.4, 3.6 Hz, 1H), 3.32 (s, 1H), 3.00 (s, 1H), 2.20-1.98 (m, 2H), 1.78 (s, 2H).

### Example 93. Synthesis of 1-(4-{3-[(4,4-difluorocyclohexyl)oxy]pyrazin-2-yl}piperazin-1-yl) prop-2-en-1-one

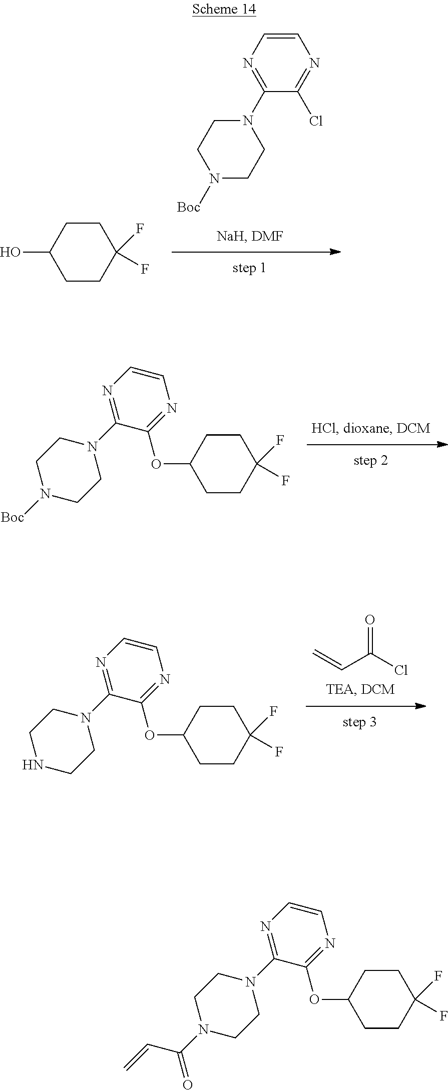

### Step 1: Synthesis of tert-butyl 4-{3-[(4,4-difluorocyclohexyl)oxy]pyrazin-2-yl}piperazine-1-carboxylate

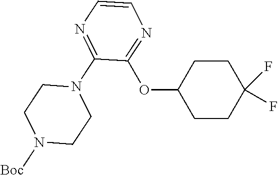 [SMILES: CC(C)(C)OC(=O)N1CCN(c2nccnc2OC2CCC(F)(F)CC2)CC1]

A solution of 4,4-difluorocyclohexan-1-ol (0.30 g, 2.20 mmol, 1.50 equiv) in DMF (1.00 mL) was treated with NaH (117.51 mg, 2.94 mmol, 2.00 equiv, 60%) for 0.5 h at 0° C. under nitrogen atmosphere followed by the addition of tert-butyl 4-(3-chloropyrazin-2-yl) piperazine-1-carboxylate (438.91 mg, 1.47 mmol, 1.00 equiv) dropwise at room temperature. The resulting mixture was stirred at rt for 3 hours before diluting with H₂O (50.00 mL). The aqueous layer was extracted with DCM (3×50.00 mL). The organic phase was washed with (4×50.00 mL) with brine and dried over anhydrous Na₂SO₄. After removing the solvent, the residue was purified by silica gel column chromatography, eluted with THE (1:10) to afford tert-butyl 4-{3-[(4,4-difluorocyclohexyl)oxy]pyrazin-2-yl}piperazine-1-carboxylate (0.30 g, 45.56%) as a white solid. LCMS ES, m/z: 399.2.

### Step 2: Synthesis of 2-((4,4-difluorocyclohexyl)oxy)-3-(piperazin-1-yl)pyrazine

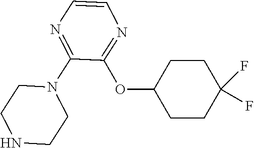 [SMILES: FC1(F)CCC(Oc2nccnc2N2CCNCC2)CC1]

A solution of tert-butyl 4-{3-[(4,4-difluorocyclohexyl)oxy]pyrazin-2-yl}piperazine-1-carboxylate (300.00 mg, 0.75 mmol, 1.00 equiv) and HCl(gas) in 1,4-dioxane (1.00 mL, 32.91 mmol, 43.71 equiv) in DCM (5.00 mL) was stirred for 1 hour at room temperature. The mixture was concentrated under vacuum to give 2-((4,4-difluorocyclohexyl)oxy)-3-(piperazin-1-yl)pyrazine (0.20 g, 89.29% yield) as white solid which was directly used in next step without further purification. LCMS ES, m/z: 299.2 Step 3: Synthesis of 1-(4-{3-[(4,4-difluorocyclohexyl)oxy]pyrazin-2-yl}piperazin-1-yl) prop-2-en-1-one

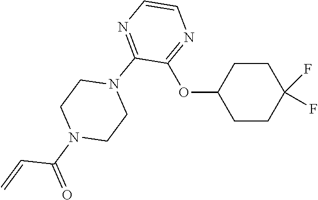 [SMILES: C=CC(=O)N1CCN(c2nccnc2OC2CCC(F)(F)CC2)CC1]

To a stirred solution of 2-[(4,4-difluorocyclohexyl)oxy]-3-(piperazin-1-yl)pyrazine (200.00 mg, 0.75 mmol, 1.00 equiv) and TEA (228.55 mg, 2.26 mmol, 3.00 equiv) in DCM (5.00 mL) were added acryloyl chloride (68.14 mg, 0.75 mmol, 1.00 equiv) dropwise at 0° C. under N₂ atmosphere. After the reaction was completed, the mixture was extracted with EA (3×15.00 mL). The combined organic layers were washed with H₂O (3×15.00 mL), dried over anhydrous Na₂SO₄. After filtration, the filtrate was concentrated under reduced pressure. The crude product (270.05 mg) was purified by Prep-HPLC with the following conditions (Column: XBridge C18, 19*150 mm, 5 m; Mobile Phase A: 20 mM NH₄HCO₃+0.05% NH₃H₂O, Mobile Phase B: ACN; Flow rate: 20 mL/min; Gradient: 10% to 65% in 8 min.) to afford 1-(4-{3-[(4,4-difluorocyclohexyl)oxy]pyrazin-2-yl}piperazin-1-yl) prop-2-en-1-one. LCMS ES, m/z: 353.2. ¹H NMR (400 MHz, DMSO-d₆): δ0.7.78-7.77 (m, 1H), 7.62-7.61 (m, 1H), 6.87-6.80 (m, 1H), 6.15-6.11 (m, 1H), 5.71-5.68 (m, 1H), 5.28-5.27 (m, 1H), 3.69-3.65 (m, 4H), 3.46 (m, 4H), 2.07-1.91 (m, 8H).

### Example 94: Synthesis of 1-(4-(3-((4-(trifluoromethyl)cyclohexyl)oxy)pyrazin-2-yl)piperazin-1-yl)prop-2-en-1-one

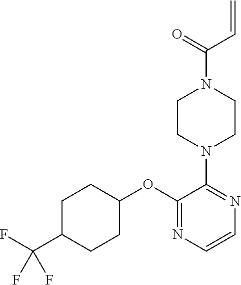 [SMILES: C=CC(=O)N1CCN(c2nccnc2OC2CCC(C(F)(F)F)CC2)CC1]

Following the general procedure in scheme 13, 4-(trifluoromethyl)cyclohexan-1-ol was used in step 1. Offered 1-(4-(3-((4-(trifluoromethyl)cyclohexyl)oxy)pyrazin-2-yl)piperazin-1-yl)prop-2-en-1-one as white solid. (Positive, ES, m/z): 385.33. ¹H NMR (400 MHz, DMSO) δ 7.77 (d, J=3.0 Hz, 1H), 7.62 (d, J=2.9 Hz, 1H), 6.84 (dd, J=16.7, 10.4 Hz, 1H), 6.15 (d, J=16.7 Hz, 1H), 5.72 (d, J=10.5 Hz, 1H), 5.00 (s, 1H), 3.67 (d, J=18.0 Hz, 4H), 3.45 (s, 4H), 2.40 (s, 1H), 2.30-2.11 (m, 2H), 2.03-1.86 (m, 2H), 1.67-1.34 (m, 4H).

### Example 95. Synthesis of 1-(4-(3-(4-(trifluoromethyl)benzoyl)pyrazin-2-yl)piperazin-1-yl)prop-2-en-1-one

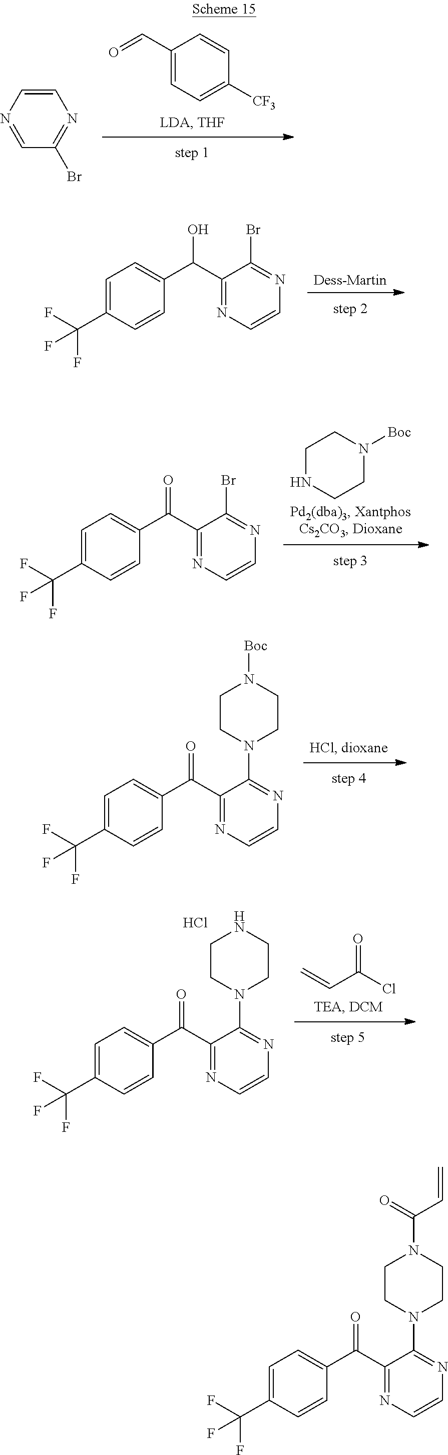

### Step 1: Synthesis of (3-bromopyrazin-2-yl)(4-(trifluoromethyl)phenyl)methanol

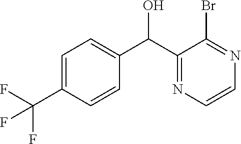 [SMILES: OC(c1ccc(C(F)(F)F)cc1)c1nccnc1Br]

A solution of 2-bromopyrazine (4 g, 25.15 mmol, 1 equiv) in THE was treated with LDA (27 mL, 199.10 mmol, 7.91 equiv) for 20 min at −78° C. under nitrogen atmosphere followed by the addition of 4-(trifluoromethyl)benzaldehyde (6.57 g, 37.73 mmol, 1.5 equiv) dropwise at −78° C. The resulting mixture was stirred for 30 min at −40° C. under nitrogen atmosphere. The resulting mixture was quenched with 100.00 mL H₂O at −40° C. and then extracted with ethyl acetate (100.00 mL×3). The combined organic layers were washed with brine (50.00 mL), dried over anhydrous Na₂SO₄. After filtration, the filtrate was concentrated under reduced pressure. The residue was purified by silica gel column chromatography, eluted with PE: EA (5:1) to afford (3-bromopyrazin-2-yl) [4-(trifluoromethyl)phenyl]methanol (4.3 g, 51.31%) as yellow solid.

### Step 2: Synthesis of (3-bromopyrazin-2-yl)(4-(trifluoromethyl)phenyl)methanone

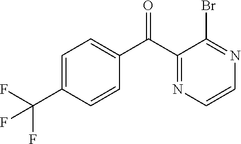 [SMILES: O=C(c1ccc(C(F)(F)F)cc1)c1nccnc1Br]

A solution of (3-bromopyrazin-2-yl) [4-(trifluoromethyl)phenyl]methanol (1 g, 3.00 mmol, 1 equiv) and Dess-Martin periodinane (2.55 g, 6.00 mmol, 2 equiv) in DCM was stirred for 2 h at room temperature under nitrogen atmosphere. The resulting mixture was added 100.00 mL H₂O and extracted with ethyl acetate (100.00 mL×3). The combined organic layers were washed with brine (50.00 mL), dried over anhydrous Na₂SO₄. After filtration, the filtrate was concentrated under reduced pressure. The residue was purified by silica gel column chromatography, eluted with PE: EA (5:1) to afford 2-bromo-3-[4-(trifluoromethyl)benzoyl]pyrazine (980 mg, 98.60%) as a white solid.

### Step 3: Synthesis of tert-butyl 4-(3-(4-(trifluoromethyl)benzoyl)pyrazin-2-yl)piperazine-1-carboxylate

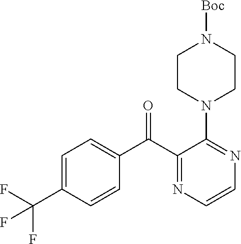 [SMILES: CC(C)(C)OC(=O)N1CCN(c2nccnc2C(=O)c2ccc(C(F)(F)F)cc2)CC1]

Into a 40 mL vial were added 2-bromo-3-[4-(trifluoromethyl)benzoyl]pyrazine (3 g, 9.06 mmol, 1 equiv), tert-butyl piperazine-1-carboxylate (2.53 g, 13.59 mmol, 1.5 equiv), XantPhos (0.47 g, 0.81 mmol, 0.09 equiv), Cs₂CO₃ (8.86 g, 27.18 mmol, 3 equiv), Pd₂(dba)₃ (0.33 g, 0.36 mmol, 0.04 equiv) and 1,4-dioxane (30 mL) at room temperature. The resulting mixture was stirred for 3 h at 80° C. under nitrogen atmosphere. The resulting mixture was added 100.00 mL H₂O and extracted with ethyl acetate (100.00 mL×3). The combined organic layers were washed with brine (50.00 mL), dried over anhydrous Na₂SO₄. After filtration, the filtrate was concentrated under reduced pressure. The residue was purified by silica gel column chromatography, eluted with PE: EA (3:1) to afford tert-butyl 4-{3-[4-(trifluoromethyl)benzoyl]pyrazin-2-yl}piperazine-1-carboxylate (3.6 g, 91.04%) as a white solid.

### Step 4: Synthesis of (3-(piperazin-1-yl)pyrazin-2-yl)(4-(trifluoromethyl)phenyl)methanone (HCl salt)

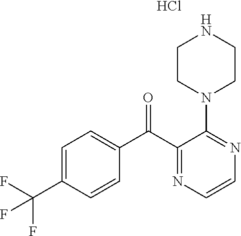 [SMILES: Cl ; O=C(c1ccc(C(F)(F)F)cc1)c1nccnc1N1CCNCC1]

Into a 40 mL vial were added tert-butyl 4-{3-[4-(trifluoromethyl)benzoyl]pyrazin-2-yl}piperazine-1-carboxylate (530 mg, 1.21 mmol, 1 equiv) and DCM (4 mL), HCl(gas)in 1,4-dioxane (2 mL) at room temperature. The resulting mixture was stirred for 1 h at room temperature under nitrogen atmosphere. The resulting mixture was concentrated under reduced pressure to afford 2-(piperazin-1-yl)-3-[4-(trifluoromethyl)benzoyl]pyrazine (400 mg, 98%) as a white solid.

### Step 5: Synthesis of 1-(4-(3-(4-(trifluoromethyl)benzoyl)pyrazin-2-yl)piperazin-1-yl)prop-2-en-1-one

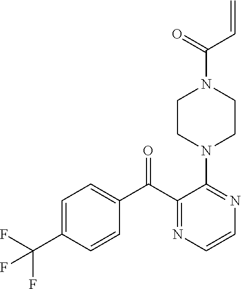 [SMILES: C=CC(=O)N1CCN(c2nccnc2C(=O)c2ccc(C(F)(F)F)cc2)CC1]

A solution of 2-(piperazin-1-yl)-3-[4-(trifluoromethyl)benzoyl]pyrazine (400 mg, 1.18 mmol, 1 equiv) in DCM was treated with TEA (361.06 mg, 3.56 mmol, 3 equiv) for 3 min at 0° C. under nitrogen atmosphere followed by the addition of acryloyl chloride (113.03 mg, 1.24 mmol, 1.05 equiv) dropwise at 0° C. The resulting mixture was stirred for 1 h at room temperature under nitrogen atmosphere. The resulting mixture was concentrated under reduced pressure. The crude product (200 mg) was purified by Prep-HPLC with the following conditions (Column: YMC-Actus Triart C18, 250*50 mm, 10 μm; Mobile Phase A: 0.1% NH₃ H₂O, Mobile Phase B: ACN; Flow rate: 80 mL/min; Gradient: 15% B-50% B-17 min) to afford to afford 1-(4-{3-[4-(trifluoromethyl)benzoyl]pyrazin-2-yl}piperazin-1-yl)prop-2-en-1-one (70 mg, 14.78%) as a yellow solid. LCMS:(ES, m/z): [M+H]⁺=391. ¹H NMR (400 MHz, DMSO-d6) δ 8.37 (d, J=2.0 Hz, 1H), 8.15 (d, J=8.0 Hz, 2H), 8.02 (d, J=2.4 Hz, 1H), 7.93 (d, J=8.0 Hz, 2H), 6.75 (d, J=10.4 Hz, 1H), 6.13 (d, J=2.4 Hz, 1H), 5.69 (d, J=2.4 Hz, 1H), 3.66 (d, J=27.8 Hz, 4H), 3.46 (d, J=23.2 Hz, 4H).

### Example 96: Synthesis of 1-(4-(3-(6-(trifluoromethyl)nicotinoyl)pyrazin-2-yl)piperazin-1-yl)prop-2-en-1-one

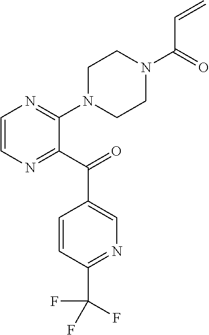 [SMILES: C=CC(=O)N1CCN(c2nccnc2C(=O)c2ccc(C(F)(F)F)nc2)CC1]

Following the general procedure in scheme 15, 6-(trifluoromethyl)nicotinaldehyde was used in step 1. Offered 1-(4-(3-(6-(trifluoromethyl)nicotinoyl)pyrazin-2-yl)piperazin-1-yl)prop-2-en-1-one as white solid. (Positive, ES, m/z): 392.22. ¹H NMR (400 MHz, DMSO) δ 9.28 (d, J=2.0 Hz, 1H), 8.65-8.53 (m, 1H), 8.42 (d, J=2.1 Hz, 1H), 8.12 (dd, J=8.2, 0.9 Hz, 1H), 8.05 (d, J=2.2 Hz, 1H), 6.79 (dd, J=16.6, 10.4 Hz, 1H), 6.16 (dd, J=16.7, 2.4 Hz, 1H), 5.72 (dd, J=10.4, 2.4 Hz, 1H), 3.83-3.62 (m, 4H), 3.64-3.44 (m, 4H).

### Example 97. Synthesis of 1-(4-(3-(hydroxy(4-(trifluoromethyl)phenyl)methyl)pyrazin-2-yl)piperazin-1-yl)prop-2-en-1-one

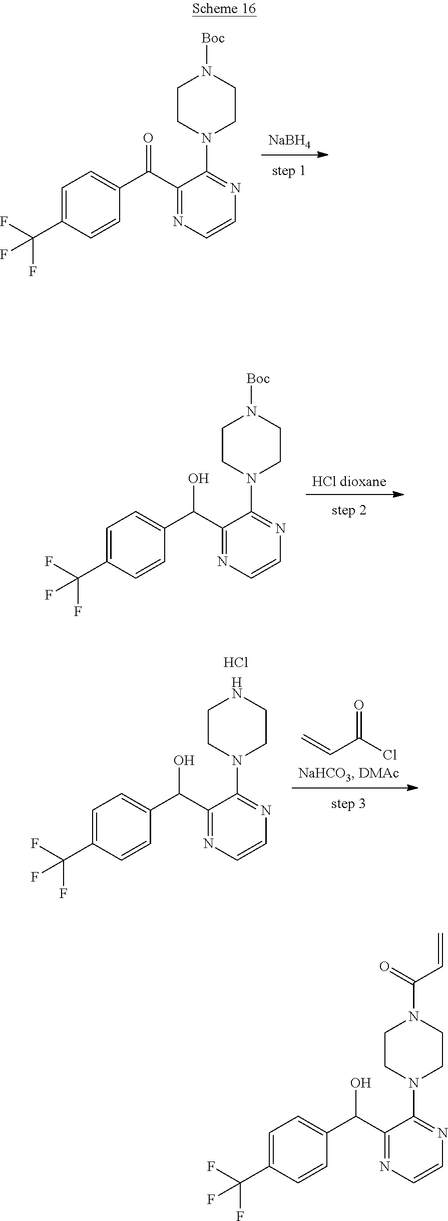

### Step 1: Synthesis of tert-butyl 4-(3-{hydroxyl [4-(trifluoromethyl)phenyl]methyl}pyrazin-2-yl) piperazine-1-carboxylate

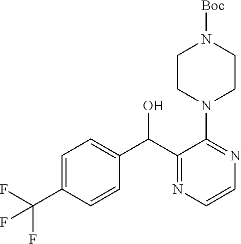 [SMILES: CC(C)(C)OC(=O)N1CCN(c2nccnc2C(O)c2ccc(C(F)(F)F)cc2)CC1]

Into a 40 mL vial were added tert-butyl 4-{3-[4-(trifluoromethyl)benzoyl]pyrazin-2-yl}piperazine-1-carboxylate (500 mg, 1.14 mmol, 1 equiv), NaBH₄ (43.34 mg, 1.14 mmol, 1 equiv) and MeOH (5 mL) at room temperature. The resulting mixture was stirred for 30 min at room temperature under nitrogen atmosphere. Desired product could be detected by LCMS. The resulting mixture was added 50 mL H₂O and extracted with ethyl acetate (50 mL×3). The combined organic layers were washed with brine (100.00 mL), dried over anhydrous Na₂SO₄. After filtration, the filtrate was concentrated under reduced pressure to afford tert-butyl 4-(3-{hydroxyl [4-(trifluoromethyl)phenyl]methyl}pyrazin-2-yl) piperazine-1-carboxylate (450 mg, crude) as a white solid. The crude product was used in the next step directly without further purification.

### Step 2: Synthesis of (3-(piperazin-1-yl)pyrazin-2-yl)(4-(trifluoromethyl)phenyl)methanol (HCl salt)

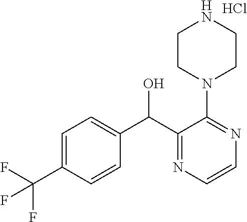 [SMILES: Cl ; OC(c1ccc(C(F)(F)F)cc1)c1nccnc1N1CCNCC1]

Into a 40 mL vial were added tert-butyl 4-(3-{hydroxy[4-(trifluoromethyl)phenyl]methyl}pyrazin-2-yl) piperazine-1-carboxylate (400 mg, 0.91 mmol, 1 equiv) and DCM (3 mL), HCl(gas) in 1,4-dioxane (4M, 1.5 mL) at room temperature. The resulting mixture was stirred for 1 h at room temperature under nitrogen atmosphere. The resulting mixture was concentrated under reduced pressure to afford 1-[4-(3-{hydroxy[4-(trifluoromethyl)phenyl]methyl}pyrazin-²-yl) piperazin-1-yl]prop-2-en-1-one (450 mg, 97.00%) as a white solid.

### Step 3: Synthesis of 1-(4-(3-(hydroxy(4-(trifluoromethyl)phenyl)methyl)pyrazin-2-yl)piperazin-1-yl)prop-2-en-1-one

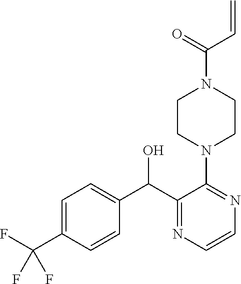 [SMILES: C=CC(=O)N1CCN(c2nccnc2C(O)c2ccc(C(F)(F)F)cc2)CC1]

A solution of [3-(piperazin-1-yl) pyrazin-2-yl][4-(trifluoromethyl)phenyl]methanol (200 mg, 0.59 mmol, 1 equiv) in DMA was treated with NaHCO₃ (148.98 mg, 1.77 mmol, 3 equiv) for 3 min at 0° C. under nitrogen atmosphere followed by the addition of acryloyl chloride (56.18 mg, 0.62 mmol, 1.05 equiv) dropwise at 0° C. The resulting mixture was stirred for 2 h at room temperature under nitrogen atmosphere. Desired product could be detected by LCMS. The resulting mixture was added 20.00 mL H₂O and extracted with ethyl acetate (50.00 mL×3). The combined organic layers were washed with brine 50.00 mL), dried over anhydrous Na₂SO₄. After filtration, the filtrate was concentrated under reduced pressure. The crude product was purified by (Column: Welch Xtimate C18 ExRS, 250 mm, 10 μm; Mobile Phase A: Water (0.05⁰% oNH₃·H₂O), Mobile Phase B: ACN; Flow rate: 90 mL/min; Gradient: 90% B to 90% B in 10 min, 90% B; Wave Length: 254 nm; RT1(min): 7; Number of Runs: 5) to afford 1-[4-(3-{hydroxy[4-(trifluoromethyl)phenyl]methyl}pyrazin-2-yl) piperazin-1-yl]prop-2-en-1-one as a yellow solid. LCMS:(ES, m/z): [M+H]⁺=393.¹H NMR (400 MHz, CDCL₃) δ8.42-8.29 (m, 2H), 7.61 (d, J=8.0 Hz, 2H), 7.50 (d, J=8.0 Hz, 2H), 6.58 (d, J=10.4 Hz, 1H), 6.34 (d, J=16.8, Hz, 1H), 6.06 (s, 1H), 5.76 (d, J=10.4 Hz, 1H), 3.72-3.58 (m, 4H), 3.22 (dt, J=10.8 Hz, 2H), 3.01 (d, J=5.2 Hz, 2H).

### Example 98. Synthesis of 1-(4-(3-(4-(trifluoromethyl)benzyl)pyrazin-2-yl)piperazin-1-yl)prop-2-en-1-one

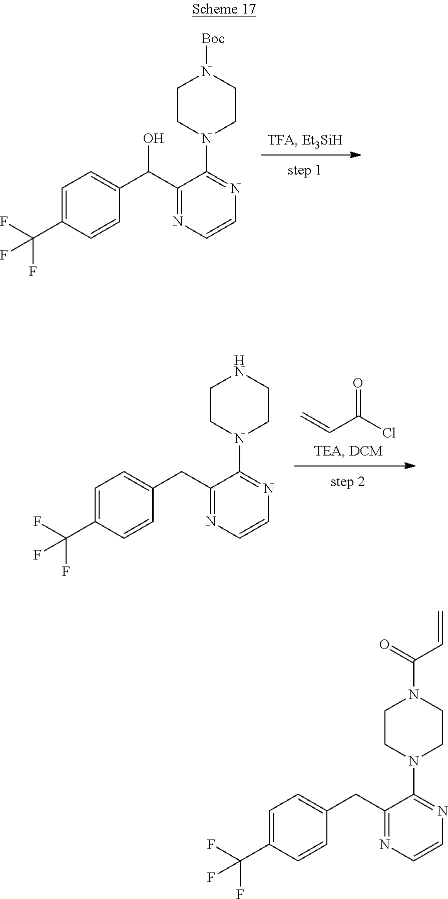

### Step 1: Synthesis of 2-(piperazin-1-yl)-3-{[4-(trifluoromethyl)phenyl]methyl}pyrazine

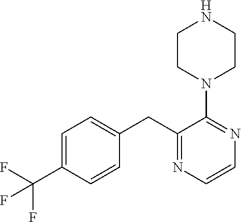 [SMILES: FC(F)(F)c1ccc(Cc2nccnc2N2CCNCC2)cc1]

A solution of tert-butyl 4-(3-{hydroxy[4-(trifluoromethyl)phenyl]methyl}pyrazin-2-yl) piperazine-1-carboxylate (400 mg, 0.91 mmol, 1 equiv) and triethylsilane (106.08 mg, 0.91 mmol, 1 equiv) in TFA was stirred for 2 h at room temperature under nitrogen atmosphere. The resulting mixture was concentrated under reduced pressure to afford 2-(piperazin-1-yl)-3-{[4-(trifluoromethyl)phenyl]methyl}pyrazine (360 mg, crude) as a white solid. The crude product was used in the next step directly without further purification.

### Step 2: Synthesis of 1-(4-(3-(4-(trifluoromethyl)benzyl)pyrazin-2-yl)piperazin-1-yl)prop-2-en-1-one

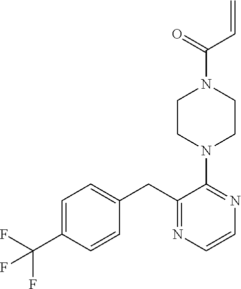 [SMILES: C=CC(=O)N1CCN(c2nccnc2Cc2ccc(C(F)(F)F)cc2)CC1]

A solution of 2-(piperazin-1-yl)-³-{[4-(trifluoromethyl)phenyl]methyl}pyrazine (300 mg, 0.93 mmol, 1 equiv) in DCM was treated with TEA (282.54 mg, 2.79 mmol, 3 equiv) for 3 min at 0° C. under nitrogen atmosphere followed by the addition of acryloyl chloride (88.45 mg, 0.97 mmol, 1.05 equiv) dropwise at 0° C. The resulting mixture was stirred for 2 h at room temperature under nitrogen atmosphere. Desired product could be detected by LCMS. The resulting mixture was added 50 mL H₂O and extracted with ethyl acetate (50 mL×3). The combined organic layers were washed with brine (100.00 mL), dried over anhydrous Na₂SO₄. After filtration, the filtrate was concentrated under reduced pressure. The crude product (100 mg) was purified by (Column: Welch Xtimate C18 ExRS, 250 mm, 10 μm; Mobile Phase A: Water (0.05% NH3.H₂O), Mobile Phase B: ACN; Flow rate: 60 mL/min; Gradient: 40% B to 60% B in 16 min, 56% B; Wave Length: 254 nm; RT1(min): 7; Number of Runs: 5) to afford 1-[4-(3-{[4-(trifluoromethyl)phenyl]methyl}pyrazin-2-yl) piperazin-1-yl]prop-2-en-1-one (70 mg, 19.98%) as a yellow oil. LCMS:(ES, m/z): [M+H]⁺=377. ¹H NMR (400 MHz, DMSO-d6) δ 8.20 (d, J=2.8 Hz, 2H), 7.65 (d, J=8.0 Hz, 2H), 7.46 (d, J=8.0 Hz, 2H), 6.85 (d, J=10.4 Hz, 1H), 6.14 (d, J=2.4 Hz, 1H), 5.71 (d, J=2.4 Hz, 1H), 4.31 (s, 2H), 3.71 (s, 4H), 3.12 (t, J=5.2 Hz, 4H).

### Example 99. Synthesis of 3-(4-acryloylpiperazin-1-yl)-N-cyclobutylpyrazine-2-carboxamide

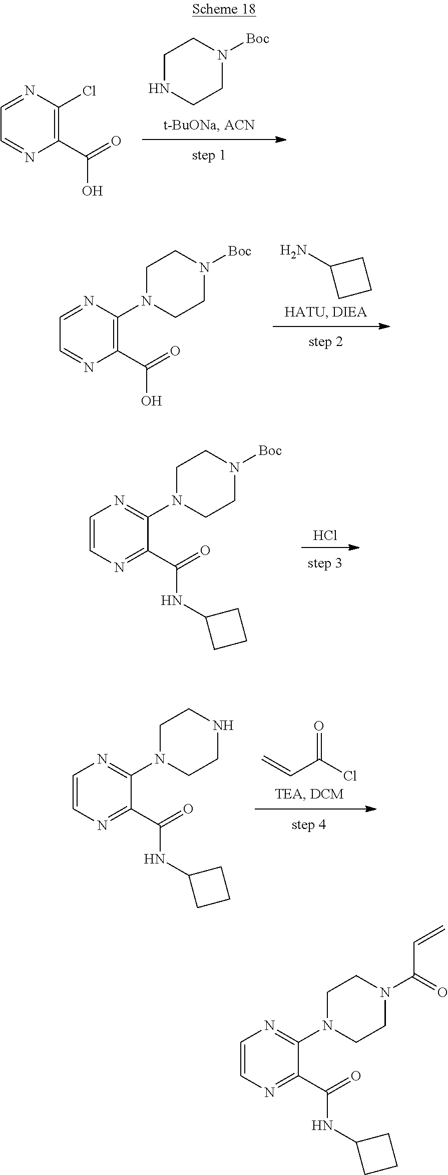

### Step 1: Synthesis of 3-[4-(tert-butoxycarbonyl) piperazin-1-yl]pyrazine-2-carboxylic acid

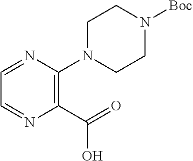 [SMILES: CC(C)(C)OC(=O)N1CCN(c2nccnc2C(=O)O)CC1]

To a stirred solution of 3-chloropyrazine-2-carboxylic acid (1580 mg, 10 mmol, 1 equiv) and tert-butyl piperazine-1-carboxylate (2790 mg, 15 mmol, 1.5 equiv) in CH₃CN (30 mL) was added t-BuONa (1920 mg, 20 mmol, 2.0 equiv) and stirred for 2 h at room temperature. The mixture was quenched with H₂O (30 mL), adjusted the pH to 5-6 with iN HCl(aq.), extracted with EA (3×50 mL). The combined organic phase was washed with brine (1×100 mL), dried over anhydrous Na₂SO₄, concentrated under reduced pressure and purified by C18 column chromatography (ACN/H₂O: 20%) to give 3-[4-(tert-butoxycarbonyl) piperazin-1-yl]pyrazine-2-carboxylic acid (2100 mg, 68.18%) as a white solid.

### Step 2: Synthesis of tert-butyl 4-[3-(cyclobutylcarbamoyl) pyrazin-2-yl]piperazine-1-carboxylate

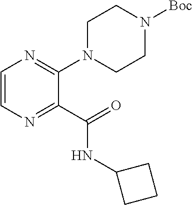 [SMILES: CC(C)(C)OC(=O)N1CCN(c2nccnc2C(=O)NC2CCC2)CC1]

To a stirred solution of 3-[4-(tert-butoxycarbonyl) piperazin-1-yl]pyrazine-2-carboxylic acid (802 mg, 2.60 mmol, 1 equiv), cyclobutylamine (222 mg, 3.12 mmol, 1.2 equiv) and DIEA (1008 mg, 7.80 mmol, 3 equiv) in DCM (8 mL) was added HATU (1186 mg, 3.12 mmol, 1.2 equiv) in portions and stirred for 1 hour at room temperature. The mixture was quenched with H₂O (30 mL) and extracted with DCM (3×50 mL). The combined organic phase was washed with brine, dried over anhydrous Na₂SO₄, concentrated under reduced pressure and purified by column chromatography on silica gel (Combiflash, PE/THF=4:1) to give tert-butyl 4-[3-(cyclobutylcarbamoyl) pyrazin-2-yl]piperazine-1-carboxylate (745 mg, 79.24%) as a white solid.

### Step 3: Synthesis of N-cyclobutyl-3-(piperazin-1-yl) pyrazine-2-carboxamide

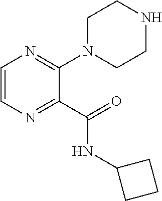 [SMILES: O=C(NC1CCC1)c1nccnc1N1CCNCC1]

To a stirred solution of tert-butyl 4-[3-(cyclobutylcarbamoyl) pyrazin-2-yl]piperazine-1-carboxylate (342 mg, 0.94 mmol, 1 equiv) in DCM (4 mL) was added HCl (gas) in 1,4-dioxane (1 mL, 4 mmol, 4.255 equiv) at room temperature and stirred for 1 hour. The resulting mixture was concentrated under reduced pressure to give N-cyclobutyl-3-(piperazin-1-yl) pyrazine-2-carboxamide (240 mg, 97.06%) as a white solid.

### Step 4: Synthesis of 3-(4-acryloylpiperazin-1-yl)-N-cyclobutylpyrazine-2-carboxamide

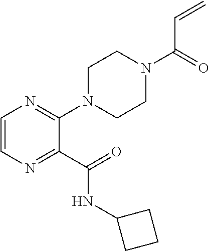 [SMILES: C=CC(=O)N1CCN(c2nccnc2C(=O)NC2CCC2)CC1]

To a stirred solution of N-cyclobutyl-3-(piperazin-1-yl) pyrazine-2-carboxamide (342 mg, 1.309 mmol, 1.0 equiv) and TEA (397.29 mg, 3.927 mmol, 3 equiv) in DCM (4 mL) were added acryloyl chloride (142.14 mg, 1.571 mmol, 1.2 equiv) dropwised at 0° C. and stirred 1 hour. The reaction was quenched with water (20 mL), and extracted with DCM (3×30 mL). The combined organic phase was washed with brine(1×50 mL), dried over anhydrous Na₂SO₄, concentrated under reduced pressure. The crude was purified by Prep-HPLC with the following conditions (Column: XBridge C18, 19*150 mm, 5 m; Mobile Phase A: 20 mM NH₄HCO₃+0.05% NH₃H₂O, Mobile Phase B: ACN; Flow rate: 20 mL/min; Gradient: 10% to 65% in 8 min.) to afford N-cyclobutyl-3-[4-(prop-2-enoyl) piperazin-1-yl]pyrazine-2-carboxamide (63 mg, 15.26%) as a white solid. LCMS (ES, m/z): [M+H]>=316. ¹H NMR (400 MHz, Chloroform-d) δ 8.20 (d, J=2.3 Hz, 1H), 7.94 (d, J=2.2 Hz, 1H), 7.84 (d, J=8.3 Hz, 1H), 6.59 (dd, J=16.8, 10.5 Hz, 1H), 6.32 (dd, J=16.8, 1.9 Hz, 1H), 5.73 (dd, J=10.5, 1.9 Hz, 1H), 4.52 (m, 1H), 3.80 (d, J=26.6 Hz, 4H), 3.57-3.50 (m, 4H), 2.48-2.36 (m, 2H), 2.08-1.93 (m, 2H), 1.84-1.75 (m, 2H).

### Example 100: Synthesis of3-(4-acryloylpiperazin-1-yl)-N-(3,3-difluorocyclobutyl)pyrazine-2-carboxamide

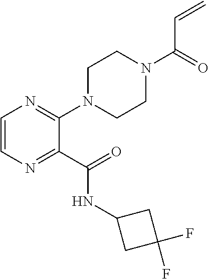 [SMILES: C=CC(=O)N1CCN(c2nccnc2C(=O)NC2CC(F)(F)C2)CC1]

Following the general procedure in scheme 18, 3,3-difluorocyclobutan-1-amine was used in step//2. Offered 3-(4-acryloylpiperazin-1-yl)-N-(3,3-difluorocyclobutyl)pyrazine-2-carboxamide as white solid. (Positive, ES, m/z): 352.36. ¹H NMR (400 MHz, DMSO) δ 9.19 (d, J=6.8 Hz, 1H), 8.27 (d, J=2.4 Hz, 1H), 8.00 (d, J=2.3 Hz, 1H), 6.83 (dd, J=16.7, 10.4 Hz, 1H), 6.15 (dd, J=16.8, 2.3 Hz, 1H), 5.72 (dd, J=10.4, 2.4 Hz, 1H), 4.31-4.13 (m, 1H), 3.72-3.58 (m, 4H), 3.45-3.43 (m, 4H), 3.32 (d, J=3.2 Hz, 1H), 2.96 (t, J=7.0 Hz, 2H), 2.87-2.67 (m, 2H).

### Example 101: Synthesis of 1-(4-(3-(6-(trifluoromethyl)-2-azaspiro[3.3]heptane-2-carbonyl)pyrazin-2-yl)piperazin-1-yl)prop-2-en-1-one

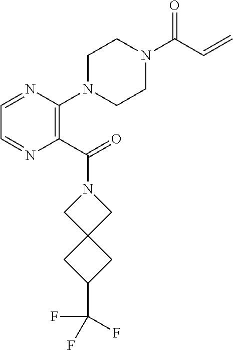 [SMILES: C=CC(=O)N1CCN(c2nccnc2C(=O)N2CC3(CC(C(F)(F)F)C3)C2)CC1]

Following the general procedure in scheme 18, 6-(trifluoromethyl)-2-azaspiro[3.3]heptane was used in step 2. Offered 1-(4-(3-(6-(trifluoromethyl)-2-azaspiro[3.3]heptane-2-carbonyl)pyrazin-2-yl)piperazin-1-yl)prop-2-en-1-one as white solid. (Positive, ES, m/z): 410.36. ¹H NMR (400 MHz, DMSO) δ 8.23 (t, J=2.6 Hz, 1H), 7.97 (t, J=2.7 Hz, 1H), 6.83 (dd, J=16.7, 10.5 Hz, 1H), 6.16 (dd, J=16.7, 2.4 Hz, 1H), 5.73 (dd, J=10.4, 2.4 Hz, 1H), 4.25-4.03 (m, 4H), 3.76-3.59 (m, 4H), 3.50-3.40 (m, 4H), 3.17-3.00 (m, 1H), 2.51-2.19 (m, 4H).

### Example 102: Synthesis of3-(4-acryloylpiperazin-1-yl)-N-cyclopentylpyrazine-2-carboxamide

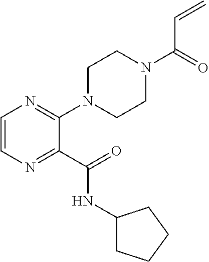 [SMILES: C=CC(=O)N1CCN(c2nccnc2C(=O)NC2CCCC2)CC1]

Following the general procedure in scheme 18, cyclopentamine was used in step 2. Offered 3-(4-acryloylpiperazin-1-yl)-N-cyclopentylpyrazine-2-carboxamide as white solid. (Positive, ES, m/z): 330.02. ¹H NMR (400 MHz, DMSO) δ 8.58 (s, 1H), 8.23 (d, J=8.3 Hz, 1H), 7.97 (d, J=8.1 Hz, 1H), 6.84 (s, 1H), 6.16 (d, J=15.3 Hz, 1H), 5.71 (s, 1H), 4.16 (s, 1H), 3.74-3.54 (m, 8H), 1.88 (s, 2H), 1.67 (s, 2H), 1.53 (s, 4H).

### Example 103: Synthesis of 1-(4-(3-(4-(trifluoromethyl)piperidine-1-carbonyl)pyrazin-2-yl)piperazin-1-yl)prop-2-en-1-one

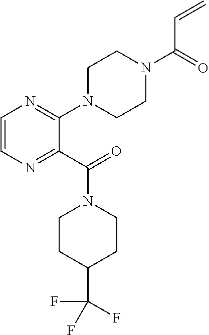 [SMILES: C=CC(=O)N1CCN(c2nccnc2C(=O)N2CCC(C(F)(F)F)CC2)CC1]

Following the general procedure in scheme 18, 4-(trifluoromethyl)piperidine was used in step 2. Offered 1-(4-(3-(4-(trifluoromethyl)piperidine-1-carbonyl)pyrazin-2-yl)piperazin-1-yl)prop-2-en-1-one as white solid. (Positive, ES, m/z): 398.32. ¹H NMR (400 MHz, DMSO) δ 8.26 (d, J=2.5 Hz, 1H), 8.02 (d, J=2.5 Hz, 1H), 6.83 (dd, J=16.7, 10.4 Hz, 1H), 6.15 (dd, J=16.7, 2.4 Hz, 1H), 5.72 (dd, 0.1=10.4, 2.4 Hz, 1H), 4.60 (d, J=13.2 Hz, 1H), 3.71-3.61 (m, 4H), 3.46 (td, J=12.8, 6.0 Hz, 4H), 3.22-3.11 (m, 1H), 2.87 (td, J=13.1, 2.9 Hz, 1H), 2.69 (s, 1H), 1.97 (d, J=12.7 Hz, 1H), 1.80 (d, J=12.9 Hz, 1H), 1.43 (pd, J=12.8, 4.5 Hz, 2H), 1.28 (t, J=2.3 Hz, 1H).

### Example 104: Synthesis of 1-(4-(3-(4,4-difluoropiperidine-1-carbonyl)pyrazin-2-yl)piperazin-1-yl)prop-2-en-1-one

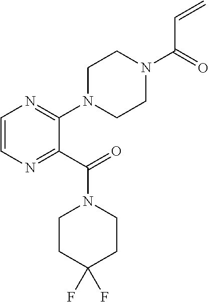 [SMILES: C=CC(=O)N1CCN(c2nccnc2C(=O)N2CCC(F)(F)CC2)CC1]

Following the general procedure in scheme 18, 4-(difluoromethyl)piperidine was used in step 2. Offered 1-(4-(3-(4,4-difluoropiperidine-1-carbonyl)pyrazin-2-yl)piperazin-1-yl)prop-2-en-1-one as white solid. (Positive, ES, m/z): 366.1. ¹H NMR (400 MHz, DMSO) δ 8.25 (s, 1H), 7.99 (s, 1H), 6.81 (d, J=14.6 Hz, 1H), 6.16 (d, J=14.8 Hz, 1H), 5.72 (s, 1H), 3.93-3.53 (m, 12H), 2.04 (s, 4H).

### Example 105: Synthesis of 1-(4-(3-(4-(trifluoromethyl)piperidin-1-yl)pyrazin-2-yl)piperazin-1-yl)prop-2-en-1-one

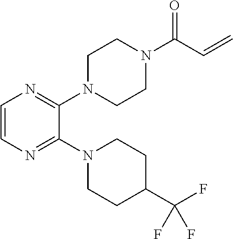 [SMILES: C=CC(=O)N1CCN(c2nccnc2N2CCC(C(F)(F)F)CC2)CC1]

Following the general procedure in scheme 1, 4-(trifluoromethyl)piperidine was used in step 2. Offered 1-(4-(3-(4-(trifluoromethyl)piperidin-1-yl)pyrazin-2-yl)piperazin-1-yl)prop-2-en-1-one as white solid. (Positive, ES, m/z): 370.35. ¹H NMR (400 MHz, DMSO) δ 7.85-7.73 (m, 2H), 6.87 (dd, J=16.6, 10.4 Hz, 1H), 6.15 (dd, J=16.6, 2.4 Hz, 1H), 5.72 (dd, J=10.4, 2.4 Hz, 1H), 4.17 (d, J=12.8 Hz, 2H), 3.85-3.62 (m, 4H), 2.66 (t, J=12.2 Hz, 2H), 1.95 (d, J=12.6 Hz, 2H), 1.64-1.43 (m, 2H).

### Example 106: Synthesis of 1-(4-(3-(3-phenylazetidin-1-yl)pyrazin-2-yl)piperazin-1-yl)prop-2-en-1-one

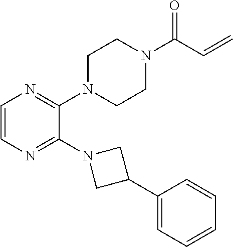 [SMILES: C=CC(=O)N1CCN(c2nccnc2N2CC(c3ccccc3)C2)CC1]

Following the general procedure in scheme 1, 3-phenylazetidine was used in step 2. Offered 1-(4-(3-(3-phenylazetidin-1-yl)pyrazin-2-yl)piperazin-1-yl)prop-2-en-1-one as white solid. (Positive, ES, m/z): 350.3. ¹H NMR (400 MHz, DMSO) δ 7.81 (d, J=2.8 Hz, 1H), 7.69 (t, J=2.5 Hz, 1H), 7.47-7.21 (m, 6H), 6.90-6.77 (m, 1H), 6.14 (ddd, J=16.9, 4.0, 2.5 Hz, 1H), 5.72 (dd, J=10.4, 2.4 Hz, 1H), 4.43 (t, J=8.2 Hz, 2H), 4.04-3.87 (m, 3H), 3.72 (d, J=14.7 Hz, 4H), 3.63 (d, J=6.7 Hz, 1H), 3.14 (s, 4H).

### Example 107: Synthesis of 1-(4-(3-(3-(4-fluorophenyl)azetidin-1-yl)pyrazin-2-yl)piperazin-1-yl)prop-2-en-1-one

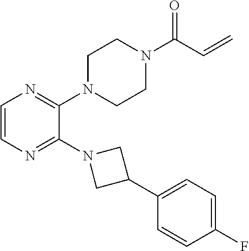 [SMILES: C=CC(=O)N1CCN(c2nccnc2N2CC(c3ccc(F)cc3)C2)CC1]

Following the general procedure in scheme 1, 3-(4-fluorophenyl)azetidine was used in step 2. Offered 1-(4-(3-(3-(4-fluorophenyl)azetidin-1-yl)pyrazin-2-yl)piperazin-1-yl)prop-2-en-1-one as white solid. (Positive, ES, m/z): 368.29. ¹H NMR (400 MHz, DMSO) δ 7.81 (d, J=2.8 Hz, 1H), 7.69 (d, J=2.8 Hz, 1H), 7.53-7.39 (m, 2H), 7.25-7.10 (m, 2H), 6.85 (dd, J=16.7, 10.5 Hz, 1H), 6.14 (dd, J=16.7, 2.4 Hz, 1H), 5.72 (dd, J=10.4, 2.4 Hz, 1H), 4.49-4.35 (m, 2H), 4.04-3.86 (m, 3H), 3.72 (d, J=13.9 Hz, 4H), 3.14 (s, 4H).

### Example 108: Synthesis of 1-(3-((3-(3-(4-fluorophenyl)azetidin-1-yl)pyrazin-2-yl)amino)azetidin-1-yl)prop-2-en-1-one

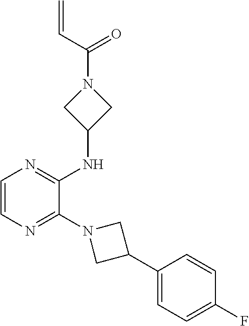 [SMILES: C=CC(=O)N1CC(Nc2nccnc2N2CC(c3ccc(F)cc3)C2)C1]

Following the general procedure in scheme 1, tert-butyl 3-aminoazetidine-1-carboxylate was used in step 1 and 3-(4-fluorophenyl)azetidine was used in step 2. Offered 1-(3-((3-(3-(4-fluorophenyl)azetidin-1-yl)pyrazin-2-yl)amino)azetidin-1-yl)prop-2-en-1-one as white solid. (Positive, ES, m/z): 354.79. ¹H NMR (400 MHz, DMSO) δ 7.48 (q, J=3.9 Hz, 4H), 7.19 (t, 0.1=8.8 Hz, 2H), 6.43 (d, 1=6.3 Hz, 1H), 6.37-6.26 (m, 1H), 6.10 (d, 0.1=17.2 Hz, 1H), 5.67 (dd, J=10.0, 2.2 Hz, 1H), 4.67-4.42 (m, 4H), 4.27-4.16 (m, 1H), 4.10-3.98 (m, 3H), 3.93 3.83 (m, 2H).

### Example 109: Synthesis of 2-fluoro-1-(3-((3-(3-(4-fluorophenyl)azetidin-1-yl)pyrazin-2-yl)amino)azetidin-1-yl)prop-2-en-1-one

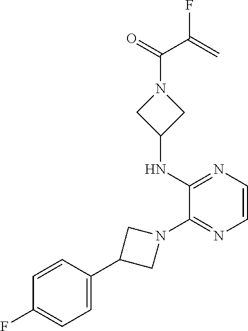 [SMILES: C=C(F)C(=O)N1CC(Nc2nccnc2N2CC(c3ccc(F)cc3)C2)C1]

Following the general procedure in scheme 10, N-(azetidin-3-yl)-3-(3-(4-fluorophenyl)azetidin-1-yl)pyrazin-2-amine was used. Offered 2-fluoro-1-(3-((3-(3-(4-fluorophenyl)azetidin-1-yl)pyrazin-2-yl)amino)azetidin-1-yl)prop-2-en-1-one as white solid. (Positive, ES, m/z): 372.71. ¹H NMR (400 MHz, DMSO) δ 7.48 (q, J=4.8 Hz, 4H), 7.19 (t, J-8.7 Hz, 2H), 6.43 (d, 0.1=5.5 Hz, 1H), 5.56-5.23 (m, 2H), 4.63 (s, 2H), 4.47 (q, I=8.8 Hz, 2H), 4.25 (d, J=7.3 Hz, 2H), 4.04 (q, J=8.0 Hz, 2H), 3.90 (q, J=7.7 Hz, 2H).

### Example 110: Synthesis of 1-(4-(3-(6-(trifluoromethyl)-2-azaspiro[3.3]heptan-2-yl)pyrazin-2-yl)piperazin-1-yl)prop-2-en-1-one

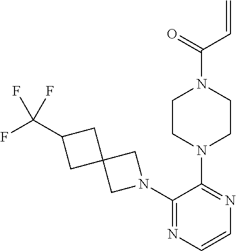 [SMILES: C=CC(=O)N1CCN(c2nccnc2N2CC3(CC(C(F)(F)F)C3)C2)CC1]

Following the general procedure in scheme 1, 6-(trifluoromethyl)-2-azaspiro[3.3]heptane was used in step 2. Offered 1-(4-(3-(6-(trifluoromethyl)-2-azaspiro[3.3]heptan-2-yl)pyrazin-2-yl)piperazin-1-yl)prop-2-en-1-one as white solid. (Positive, ES, m/z): 382.39. ¹H NMR (400 MHz, DMSO) δ 7.76 (d, J=2.8 Hz, 1H), 7.64 (d, J=2.8 Hz, 1H), 6.85 (dd, J=16.7, 10.5 Hz, 1H), 6.15 (dd, J=16.7, 2.4 Hz, 1H), 5.72 (dd, J=10.5, 2.4 Hz, 1H), 4.04 (s, 2H), 3.96 (s, 2H), 3.72 (d, J=13.5 Hz, 4H), 3.09 (s, 5H), 2.46 (dd, J=13.1, 9.1 Hz, 2H), 2.30 (dd, J=13.1, 7.4 Hz, 2H).

### Example 111. Synthesis of 1-(4-(5-hydroxy-3-((4-(trifluoromethyl)phenyl)amino)pyrazin-2-yl)piperazin-1-yl)prop-2-en-1-one

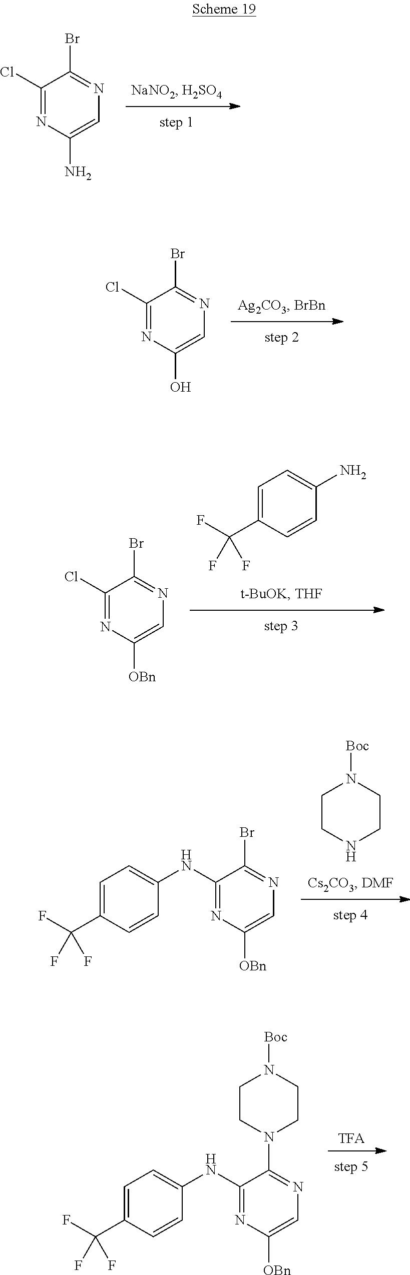

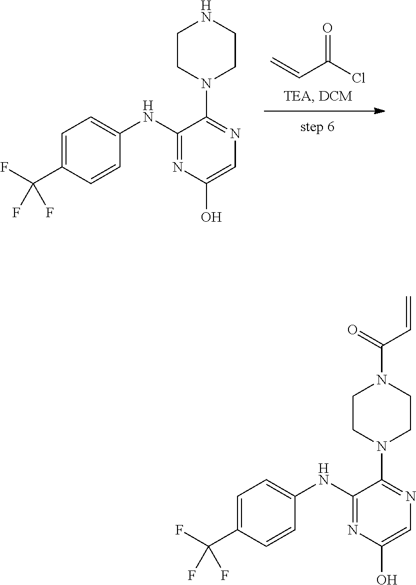

### Step 1: Synthesis of 5-bromo-6-chloropyrazin-2-ol

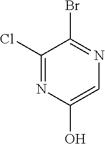

To a stirred solution of 5-bromo-6-chloropyrazin-2-amine (10 g, 48.5 mmol, 1.00 equiv) in H2SO₄ (60 mL) was added NaNO2 (3.68 g, 53.3 mmol, 1.10 equiv) in portions at 0° C. The resulting mixture was stirred for 1 h at 0° C. The reaction was quenched by the addition of ice-water (200 mL) at 0° C. The precipitated solids were collected by filtration and washed with water (2×300 mL), then dried to give 5-bromo-6-chloropyrazin-2-ol (5.5 g, 54.78%) as an off-white solid.

### Step 2: Synthesis of 5-(benzyloxy)-2-bromo-3-chloropyrazine

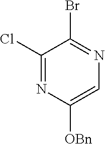 [SMILES: Clc1nc(OCc2ccccc2)cnc1Br]

To a stirred solution of 5-bromo-6-chloropyrazin-2-ol (5.4 g, 25.784 mmol, 1 equiv) and benzyl bromide (8.82 g, 51.568 mmol, 2 equiv) in toluene (60 mL) was added Ag₂CO₃ (7.47 g, 27.073 mmol, 1.05 equiv) at 0° C. This resulting was stirred at room temperature for 3 h. The resulting mixture was concentrated under reduced pressure. The residue was dissolved in water (150 mL) and extracted with EtOAc (3×100 mL). The combined organic phase was washed with brine (1×100 mL), dried over anhydrous Na₂SO₄ and concentrated under reduced pressure. The residue was purified by column chromatography on silica gel (THF/PE=25%) to give 5-(benzyloxy)-2-bromo-3-chloropyrazine (6 g, 77.68%) as a white solid.

### Step 3: Synthesis of 6-(benzyloxy)-3-bromo-N-[4-(trifluoromethyl)phenyl]pyrazin-2-amine

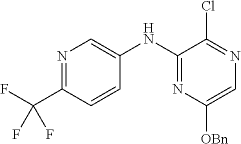 [SMILES: FC(F)(F)c1ccc(Nc2nc(OCc3ccccc3)cnc2Cl)cn1]

To a stirred solution of 5-(benzyloxy)-2-bromo-3-chloropyrazine (2.512 g, 8.386 mmol, 1 equiv) and t-BuOK (1.88 g, 16.772 mmol, 2 equiv) in DMAc (50 mL) was added p-trifluoromethylaniline (1.62 g, 10.063 mmol, 1.2 equiv) at room temperature. The resulting was stirred for 2 hours at 100° C. To the mixture was added water (200 mL), the aqueous layer was extracted with EA (3×200 mL). The combined organic phase was washed with brine (1×100 mL) and concentrated. The residue was purified by silica gel column chromatography, eluted with (PE/THF: 20%) to afford 6-(benzyloxy)-3-bromo-N-[4-(trifluoromethyl)phenyl]pyrazin-2-amine (1.945 g, 54.67%) as a yellow solid. LCMS-PH-CAL-Y-V2475860-3:(ES, m/z): [M+H]-=424

### Step 4: Synthesis of tert-butyl 4-[5-(benzyloxy)-3-{[4-(trifluoromethyl)phenyl]amino}pyrazin-2-yl]piperazine-1-carboxylate

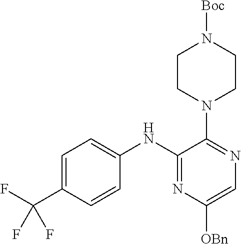 [SMILES: CC(C)(C)OC(=O)N1CCN(c2ncc(OCc3ccccc3)nc2Nc2ccc(C(F)(F)F)cc2)CC1]

To a stirred solution of 6-(benzyloxy)-3-bromo-N-[4-(trifluoromethyl)phenyl]pyrazin-2-amine (1.945 g, 4.585 mmol, 1 equiv) and tert-butyl piperazine-1-carboxylate (1.02 g, 5.502 mmol, 1.2 equiv) in DMF (20 mL) was added Cs₂CO₃ (2.99 g, 9.170 mmol, 2 equiv), Pa₂(dba)₃(0.41 g, 0.45 mmol, 0.1 equiv) and Xphos at room temperature. The resulting mixture was stirred for 3 hours at 100° C. To the mixture was added water (60 mL) and extracted with EA (3×60 mL), washed with brine (2×60 mL), dried over anhydrous Na₂SO₄. The organic phase was concentrated and purified by column chromatography on silica gel (PE/THF: 25%) to give tert-butyl 4-[5-(benzyloxy)-3-{[4-(trifluoromethyl)phenyl]amino}pyrazin-2-yl]piperazine-1-carboxylate (700 mg, 28.83%) as a yellow solid. LCMS (ES, m/z): [M+H]⁺=530

### Step 5: Synthesis of 5-(piperazin-1-yl)-6-{[4-(trifluoromethyl)phenyl]amino}pyrazin-2-ol

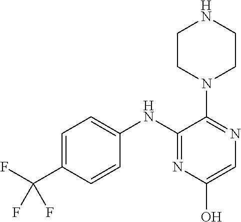 [SMILES: Oc1cnc(N2CCNCC2)c(Nc2ccc(C(F)(F)F)cc2)n1]

To a solution of tert-butyl 4-[5-(benzyloxy)-3-{[4-(trifluoromethyl)phenyl]amino}pyrazin-2-yl]piperazine-1-carboxylate (700 mg, 1.322 mmol, 1 equiv) in DCM (8 mL) was added TFA (2 mL) and stirred for 1 hour at 80° C. The resulting mixture was concentrated under reduced pressure to give 5-(piperazin-1-yl)-6-{[4-(trifluoromethyl)phenyl]amino}pyrazin-2-ol (440 mg, 98.10%) as a brown oil. LCMS (ES, m/z):[M+H]⁺=340

### Step 6: Synthesis of 1-(4-(5-hydroxy-3-((4-(trifluoromethyl)phenyl)amino)pyrazin-2-yl)piperazin-1-yl)prop-2-en-1-one

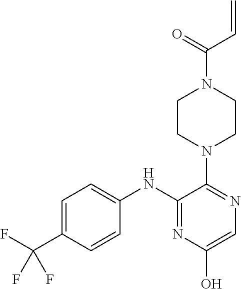 [SMILES: C=CC(=O)N1CCN(c2ncc(O)nc2Nc2ccc(C(F)(F)F)cc2)CC1]

To a stirred solution of 5-(piperazin-1-yl)-6-{[4-(trifluoromethyl)phenyl]amino}pyrazin-2-ol (400 mg, 1.179 mmol, 1 equiv) in DCM (10 mL) was added acryloyl chloride (128.03 mg, 1.415 mmol, 1.2 equiv) and TEA (357.87 mg, 3.537 mmol, 3 equiv) dropwised at 0° C., then stirred for 1 hour at room temperature. The resulting solution was stirred for 3 hours at 100° C. To the mixture was added water (20 mL), the aqueous layer was extracted with DCM (3×30 mL). The combined organic phase was dried over Na₂SO₄ and concentrated under reduced pressure, then purified by Prep-HPLC with the following conditions (Column: XBridge C18, 19*150 mm, 5 μm; Mobile Phase A: 20 mM NH₄HCO₃+0.05% NH₃H₂O, Mobile Phase B: ACN; Flow rate: 20 mL/min; Gradient: 10% to 65% in 8 min.) to afford 1-[4-(5-hydroxy-3-{[4-(trifluoromethyl)phenyl]amino}pyrazin-2-yl)piperazin-1-yl]prop-2-en-1-one (2.5 mg, 0.54%) as a yellow solid. LCMS (ES, m/z): [M+H]-=394. ¹H NMR (400 MHz, DMSO-d₆) b 10.70 (s, 1H), 8.46 (s, 1H), 8.02 (d, J=8.5 Hz, 2H), 7.63 (d, J=8.5 Hz, 2H), 7.31 (s, 1H), 6.85 (dd, J=16.7, 10.5 Hz, 1H), 6.14 (dd, J=16.5, 2.4 Hz, 1H), 5.71 (dd, J=10.4, 2.4 Hz, 1H), 3.79 (s, 4H), 2.92 (s, 4H).

### Example 112: Synthesis of 1-(4-(6-methyl-3-((4-(trifluoromethyl)phenyl)amino)pyrazin-2-yl)piperazin-1-yl)prop-2-en-1-one

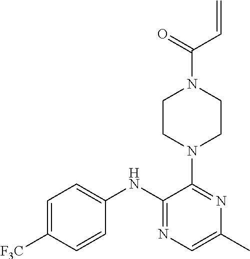 [SMILES: C=CC(=O)N1CCN(c2nc(C)cnc2Nc2ccc(C(F)(F)F)cc2)CC1]

Following the general procedure in scheme 3, 3-chloro-5-methylpyrazin-2-amine was used in step 1. Offered 1-(4-(6-methyl-3-((4-(trifluoromethyl)phenyl)amino)pyrazin-2-yl)piperazin-1-yl)prop-2-en-1-one as white solid. (Positive, ES, m/z): 322.29. ¹H NMR (400 MHz, DMSO) δ 8.52 (s, 1H), 7.88-7.74 (m, 3H), 7.62 (d, J=8.5 Hz, 2H), 6.85 (dd, J=16.7, 10.5 Hz, 1H), 6.15 (dd, J=16.7, 2.4 Hz, 1H), 5.72 (dd, J=10.4, 2.4 Hz, 1H), 3.77 (d, J=13.6 Hz, 4H), 3.14 (s, 4H), 2.33 (s, 3H).

### Example 113: Synthesis of 1-(4-(5,6-dimethyl-3-((6-(trifluoromethyl)pyridin-3-yl)amino)pyrazin-2-yl)piperazin-1-yl)prop-2-en-1-one

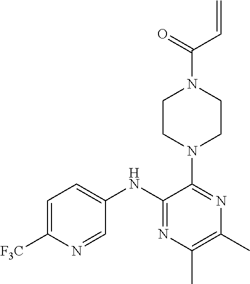 [SMILES: C=CC(=O)N1CCN(c2nc(C)c(C)nc2Nc2ccc(C(F)(F)F)nc2)CC1]

Following the general procedure in scheme 1, 2,3-dichloro-5,6-dimethylpyrazine was used in step 1. Offered 1-(4-(5,6-dimethyl-3-((6-(trifluoromethyl)pyridin-3-yl)amino)pyrazin-2-yl)piperazin-1-yl)prop-2-en-1-one as white solid. (Positive, ES, m/z): 407.58. ¹H NMR (400 MHz, DMSO) δ 9.04 (d, J=2.5 Hz, 1H), 8.71 (s, 1H), 8.40-8.26 (m, 1H), 7.79 (d, J=8.7 Hz, 1H), 6.85 (dd, J=16.7, 10.4 Hz, 1H), 6.15 (dd, J=16.8, 2.4 Hz, 1H), 5.72 (dd, J=10.5, 2.4 Hz, 1H), 3.76 (d, J=13.1 Hz, 4H), 3.08 (s, 4H), 2.35 (d, J=5.2 Hz, 6H).

### Example 114: Synthesis of 1-(4-(5,6-dimethyl-3-((6-(trifluoromethyl)pyridin-3-yl)amino)pyrazin-2-yl)piperazin-1-yl)-2-fluoroprop-2-en-1-one

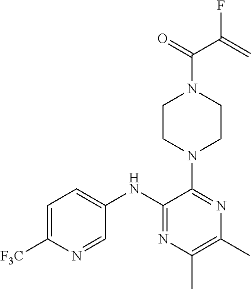 [SMILES: C=C(F)C(=O)N1CCN(c2nc(C)c(C)nc2Nc2ccc(C(F)(F)F)nc2)CC1]

Following the general procedure in scheme 10, 5,6-dimethyl-3-(piperazin-1-yl)-N-(6-(trifluoromethyl)pyridin-3-yl)pyrazin-2-amine was used. Offered 1-(4-(5,6-dimethyl-3-((6-(trifluoromethyl)pyridin-3-yl)amino)pyrazin-2-yl)piperazin-1-yl)-2-fluoroprop-2-en-1-one as white solid. (Positive, ES, m/z): 425.49. ¹H NMR (400 MHz, DMSO) δ 9.04 (s, IH), 8.71 (s, 1H), 8.34 (d, J=8.4 Hz, 1H), 7.79 (d, J=8.7 Hz, 1H), 5.38-5.12 (m, 2H), 3.74 (d, J=5.7 Hz, 4H), 3.12 (s, 4H), 2.35 (d, J=5.7 Hz, 6H).

### Example 115: Synthesis of 2-fluoro-1-(4-(6-methyl-3-((6-(trifluoromethyl)pyridin-3-yl)oxy)pyrazin-2-yl)piperidin-1-yl)prop-2-en-1-one

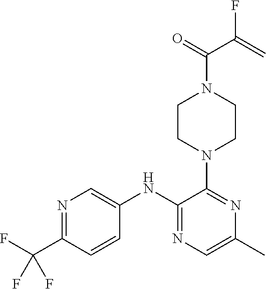 [SMILES: C=C(F)C(=O)N1CCN(c2nc(C)cnc2Nc2ccc(C(F)(F)F)nc2)CC1]

Following the general procedure in scheme 10, 5-methyl-3-(piperidin-4-yl)-2-((6-(trifluoromethyl)pyridin-3-yl)oxy)pyrazine was used. Offered 2-fluoro-1-(4-(6-methyl-3-((6-(trifluoromethyl)pyridin-3-yl)oxy)pyrazin-2-yl)piperidin-1-yl)prop-2-en-1-one as white solid. (Positive, ES, m/z): 411.62. ¹H NMR (400 MHz, DMSO) δ 8.73 (d, J=2.5 Hz, 1H), 8.09-7.90 (m, 3H), 5.33-5.10 (m, 2H), 4.41 (s, 1H), 4.04 (s, 1H), 3.52-3.46 (m, 2H), 2.97 (s, 1H), 2.46 (s, 3H), 2.01 (d, J=13.2 Hz, 2H), 1.77 (s, 2H).

### Example 116: Synthesis of 1-(4-(5-methyl-3-((6-(trifluoromethyl)pyridin-3-yl)oxy)pyrazin-2-yl)piperidin-1-yl)prop-2-en-1-one

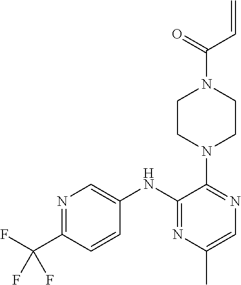 [SMILES: C=CC(=O)N1CCN(c2ncc(C)nc2Nc2ccc(C(F)(F)F)nc2)CC1]

Following the general procedure in scheme 7, tert-butyl 4-(5-methyl-3-((6-(trifluoromethyl)pyridin-3-yl)oxy)pyrazin-2-yl)piperidine-1-carboxylate was used in step 1. Offered 1-(4-(5-methyl-3-((6-(trifluoromethyl)pyridin-3-yl)oxy)pyrazin-2-yl)piperidin-1-yl)prop-2-en-1-one as white solid. (Positive, ES, m/z):393.79. ¹H NMR (400 MHz, DMSO) δ 8.76 (d, J=2.4 Hz, 1H), 8.30 (s, 1H), 8.08-7.91 (m, 2H), 6.94-6.80 (m, 1H), 6.12 (ddd, J=16.7, 2.4, 1.0 Hz, 1H), 5.69 (ddd, J=10.4, 2.5, 1.0 Hz, 1H), 4.56 (d, J=13.0 Hz, 1H), 4.20 (d, J=13.7 Hz, 1H), 3.33-3.19 (m, 2H), 2.83 (t, J=12.6 Hz, 1H), 2.31 (s, 3H), 1.97 (d, J=13.1 Hz, 2H), 1.84-1.55 (m, 2H).

### Example 117: Synthesis of 2-fluoro-1-(4-(5-methyl-3-((6-(trifluoromethyl)pyridin-3-yl)oxy)pyrazin-2-yl)piperidin-1-yl)prop-2-en-1-one

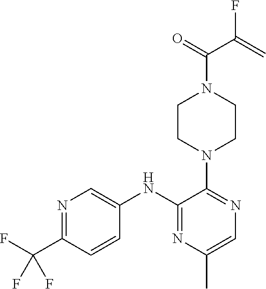 [SMILES: C=C(F)C(=O)N1CCN(c2ncc(C)nc2Nc2ccc(C(F)(F)F)nc2)CC1]

Following the general procedure in scheme 10, 5-methyl-2-(piperidin-4-yl)-3-((6-(trifluoromethyl)pyridin-3-yl)oxy)pyrazine was used. Offered 2-fluoro-1-(4-(5-methyl-3-((6-(trifluoromethyl)pyridin-3-yl)oxy)pyrazin-2-yl)piperidin-1-yl)prop-2-en-1-one as white solid. (Positive, ES, m/z): 411.81. ¹H NMR (400 MHz, DMSO) δ 8.76 (d, J=2.3 Hz, 1H), 8.31 (s, 1H), 8.08-7.94 (m, 2H), 5.33-5.07 (m, 2H), 4.40 (s, 1H), 4.03 (s, 1H), 3.54-3.45 (m, 2H), 3.06-2.87 (m, 1H), 2.31 (s, 3H), 2.12-1.95 (m, 2H), 1.75 (s, 2H).

### Example 118. Synthesis of 3-(1-acryloylpiperidin-4-yl)-1-(5-(trifluoromethyl)pyrimidin-2-yl)pyrazin-2(1H)-one

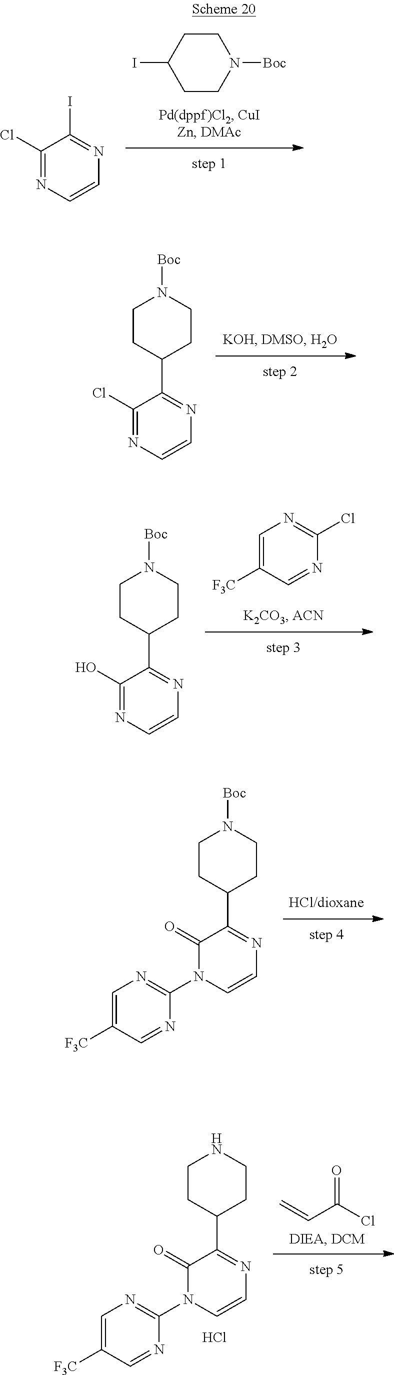

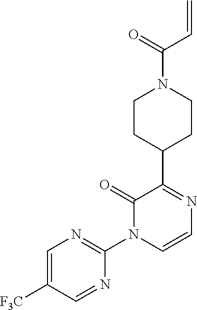 [SMILES: C=CC(=O)N1CCC(c2nccn(-c3ncc(C(F)(F)F)cn3)c2=O)CC1]

### Step 1: Synthesis of tert-butyl 4-(3-chloropyrazin-2-yl) piperidine-1-carboxylate

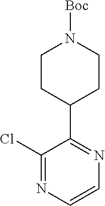 [SMILES: CC(C)(C)OC(=O)N1CCC(c2nccnc2Cl)CC1]

A solution of tert-butyl 4-iodopiperidine-1-carboxylate (3.11 g, 9.98 mmol, 1.2 equiv) and Zn (0.82 g, 12.47 mmol, 1.5 equiv) in DMAc (15 mL) was stirred for 0.5 h at 60° C. under nitrogen atmosphere. A solution of 2-chloro-3-iodopyrazine (2 g, 8.31 mmol, 1 equiv), CuI (0.32 g, 1.66 mmol, 0.2 equiv) and Pd(dppf)C₁₂ (0.61 g, 0.83 mmol, 0.1 equiv) in DMAC (15 mL) was stirred for 0.5 h at 60° C. Then the Zn solution was added to the CuI solution at 60° C. The resulting mixture was stirred for 16 h at 60° C. The resulting mixture was diluted with water (150 mL). The resulting mixture was extracted with EtOAc (3×100 mL). The combined organic layers were washed with brine (3×200 mL), dried over anhydrous Na₂SO₄. After filtration, the filtrate was concentrated under reduced pressure. The residue was purified by silica gel column chromatography, eluted with PE/THF (4:1) to afford tert-butyl 4-(3-chloropyrazin-2-yl) piperidine-1-carboxylate (1.4 g, 56.52%) as a yellow solid. LCMS (ES, m/z):[M+H]⁺=298

### Step 2: Synthesis of tert-butyl 4-(3-hydroxypyrazin-2-yl) piperidine-1-carboxylate

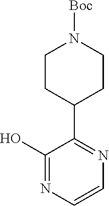 [SMILES: CC(C)(C)OC(=O)N1CCC(c2nccnc2O)CC1]

A solution of tert-butyl 4-(3-chloropyrazin-2-yl) piperidine-1-carboxylate (1.2 g, 4.03 mmol, 1 equiv) and KOH (3.39 g, 60.45 mmol, 15 equiv) in DMSO (20 mL) and H₂O (5 mL) was stirred for 2 h at 80° C. The resulting mixture was diluted with water (60 mL). The mixture was acidified to pH-3 with citric acid solution (4M, 40 mL). The resulting mixture was extracted with EtOAc (3×100 mL). The combined organic layers were washed with brine (1×100 mL), dried over anhydrous Na₂SO₄. After filtration, the filtrate was concentrated under reduced pressure. This resulted in tert-butyl 4-(3-hydroxypyrazin-2-yl) piperidine-1-carboxylate (1 g, 88.83%) as a yellow solid. LCMS (ES, m/z):[M+H]⁺=280.

### Step 3: Synthesis of tert-butyl 4-(3-oxo-4-[5-(trifluoromethyl)pyrimidin-2-yl]pyrazin-2-ylpiperidine-1-carboxylate

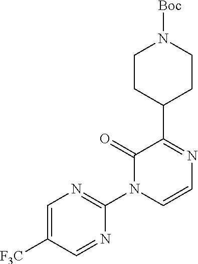 [SMILES: CC(C)(C)OC(=O)N1CCC(c2nccn(-c3ncc(C(F)(F)F)cn3)c2=O)CC1]

To a stirred solution of tert-butyl 4-(3-hydroxypyrazin-2-yl) piperidine-1-carboxylate (0.8 g, 2.86 mmol, 1 equiv) in ACN (8 mL) were added K₂CO₃ (1.19 g, 8.59 mmol, 3 equiv) and 2-chloro-5-(trifluoromethyl)pyrimidine (0.63 g, 3.43 mmol, 1.2 equiv) at room temperature. The resulting mixture was stirred for 4 h at 80° C. Two peaks with desired Ms were show in LCMS. The second peak was the desired product. The resulting mixture was diluted with water (50 mL). The resulting mixture was extracted with EtOAc (3×50 mL). The combined organic layers were washed with brine (1×50 mL), dried over anhydrous Na₂SO₄. After filtration, the filtrate was concentrated under reduced pressure. The crude product was purified by reverse phase flash with the following conditions (XBridge C18, 19*150 mm, 5 μm; Mobile Phase A: 20 mM+0.05⁰% NH₃H₂O, Mobile Phase B: ACN; Flow rate: 20 mL/min; Gradient: 10% to 65% in 8 min.) to afford tert-butyl 4-(3-oxo-4-[5-(trifluoromethyl)pyrimidin-2-yl]pyrazin-2-ylpiperidine-1-carboxylate (700 mg, 57.46%) as a yellow solid. LCMS (ES, m/z):[M+H]=426. ¹H NMR (400 MHz, DMSO-d₆) δ 9.54 (s, 2H), 7.73 (d, J=4.6 Hz, 1H), 7.39 (d, J=4.6 Hz, 1H), 4.04 (m, 2H), 3.26 (m, 1H), 2.86 (m, 2H), 1.88-1.79 (m, 2H), 1.49 (m, 2H), 1.41 (s, 9H).

### Step 4: Synthesis of 3-(piperidin-4-yl)-1-[5-(trifluoromethyl)pyrimidin-2-yl]pyrazin-2-one

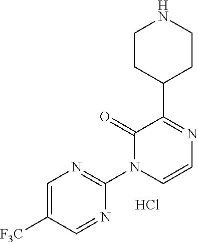 [SMILES: O=c1c(C2CCNCC2)nccn1-c1ncc(C(F)(F)F)cn1 ; Cl]

To a stirred solution of tert-butyl 4-(3-oxo-4-[5-(trifluoromethyl)pyrimidin-2-yl]pyrazin-2-ylpiperidine-1-carboxylate (700 mg, 1.645 mmol, 1 equiv) in DCM (2 mL) was added HCl/dioxane (2 mL, 8.00 mmol, 4.86 equiv) at room temperature. The resulting mixture was stirred for 1 h at rt. After the reaction was completed. The resulting mixture was concentrated under reduced pressure. This resulted in 3-(piperidin-4-yl)-1-[5-(trifluoromethyl)pyrimidin-2-yl]pyrazin-2-one (500 mg, 93.41%) as a white solid. LCMS (ES, m/z): [M+H]⁺=326

### Step 5: Synthesis of 3-[1-(prop-2-enoyl)piperidin-4-yl]-1-[5-(trifluoromethyl)pyrimidin-2-yl]pyrazin-2-one

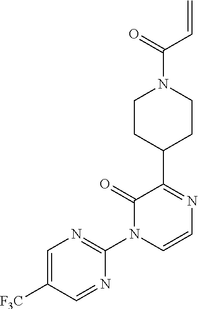 [SMILES: C=CC(=O)N1CCC(c2nccn(-c3ncc(C(F)(F)F)cn3)c2=O)CC1]

To a stirred solution of 3-(piperidin-4-yl)-1-[5-(trifluoromethyl)pyrimidin-2-yl]pyrazin-2-one (300 mg, 0.92 mmol, 1 equiv) in DCM (5 mL) were added DIEA (595.98 mg, 4.61 mmol, 5 equiv) and acryloyl chloride (166.94 mg, 1.84 mmol, 2 equiv) at 0° C. The resulting mixture was stirred for 1 h at rt. The resulting mixture was diluted with water (150 mL). The resulting mixture was extracted with DCM (3×100 mL). The combined organic layers were washed with brine (3×200 mL), dried over anhydrous Na₂SO₄. After filtration, the filtrate was concentrated under reduced pressure. The crude product was purified by reverse phase flash with the following conditions (XBridge C18, 19*150 mm, 5 i m; Mobile Phase A: 20 mM 0.05% NH₃H₂O, Mobile Phase B: ACN; Flow rate: 20 mL/min; Gradient: 10% to 65% in 8 min.) to afford 3-[1-(prop-2-enoyl)piperidin-4-yl]-1-[5-(trifluoromethyl)pyrimidin-2-yl]pyrazin-2-one (80 mg, 22.87%) as a white solid. LCMS (ES, m/z): [M+H]⁺=380. ¹H NMR (400 MHz, DMSO-d₆) δ 9.54 (s, 2H), 7.73 (d, J=4.6 Hz, 1H), 7.38 (d, J=4.6 Hz, 1H), 6.83 (dd, J=16.7, 10.5 Hz, 1H), 6.10 (dd, J=16.7, 2.4 Hz, 1H), 5.67 (dd, J=10.5, 2.5 Hz, 1H), 4.50 (d, J=12.7 Hz, 1H), 4.15 (m, 1H), 3.40 (m, 1H), 3.19 (m, 1H), 2.79 (m, 1H), 1.91 (m, 2H), 1.52 (m, 2H).

### Example 119: Synthesis of 1-(4-(3-((4-(trifluoromethyl)phenyl)amino)pyridazin-4-yl)piperazin-1-yl)prop-2-en-1-one

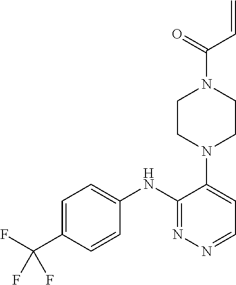 [SMILES: C=CC(=O)N1CCN(c2ccnnc2Nc2ccc(C(F)(F)F)cc2)CC1]

Following the general procedure in scheme 3, 4-chloropyridazin-3-amine was used in step 1. Offered 1-(4-(3-((4-(trifluoromethyl)phenyl)amino)pyridazin-4-yl)piperazin-1-yl)prop-2-en-1-one as white solid. (Positive, ES, m/z): 378.28. ¹H NMR (400 MHz, DMSO) δ 8.68 (d, J=5.2 Hz, 1H), 8.55 (s, 1H), 7.88 (d, J=8.5 Hz, 2H), 7.66 (d, J=8.5 Hz, 2H), 7.15 (d, J=5.3 Hz, 1H), 6.85 (dd, J=16.7, 10.5 Hz, 1H), 6.16 (dd, J=16.7, 2.4 Hz, 1H), 5.73 (dd, J=10.4, 2.4 Hz, 1H), 3.77 (d, J=13.8 Hz, 4H), 3.04 (t, J=4.9 Hz, 4H).

### Example 120: Synthesis of 1-(4-(3-((5-(trifluoromethyl)pyridin-2-yl)amino)pyridazin-4-yl)piperazin-1-yl)prop-2-en-1-one

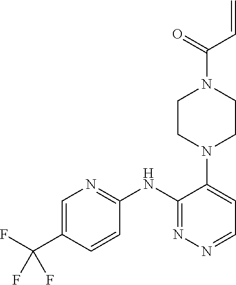 [SMILES: C=CC(=O)N1CCN(c2ccnnc2Nc2ccc(C(F)(F)F)cn2)CC1]

Following the general procedure in scheme 3, 4-chloropyridazin-3-amine and 2-iodo-5-(trifluoromethyl)pyridine were used in step 1. Offered 1-(4-(3-((5-(trifluoromethyl)pyridin-2-yl)amino)pyridazin-4-yl)piperazin-1-yl)prop-2-en-1-one as white solid. (Positive, ES, m/z): 379.65. ¹H NMR (400 MHz, DMSO) δ 9.12 (s, 1H), 8.78 (d, J=5.4 Hz, 1H), 8.61 (s, 1H), 8.10 (d, J=8.9 Hz, 1H), 7.96 (d, J=8.9 Hz, 1H), 7.22 (d, J=5.4 Hz, 1H), 6.84 (dd, J=16.7, 10.4 Hz, 1H), 6.15 (d, J=17.0 Hz, 1H), 5.77-5.66 (m, 1H), 3.68 (d, J=15.1 Hz, 4H), 3.09 (d, J=5.6 Hz, 4H).

### Example 121: Synthesis of 1-(4-(3-((6-(trifluoromethyl)pyridin-3-yl)amino)pyridazin-4-yl)piperazin-1-yl)prop-2-en-1-one

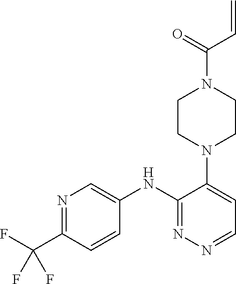 [SMILES: C=CC(=O)N1CCN(c2ccnnc2Nc2ccc(C(F)(F)F)nc2)CC1]

Following the general procedure in scheme 3, 4-chloropyridazin-3-amine and 5-iodo-2-(trifluoromethyl)pyridine were used in step 1. Offered 1-(4-(3-((5-(trifluoromethyl)pyridin-2-yl)amino)pyridazin-4-yl)piperazin-1-yl)prop-2-en-1-one as white solid. (Positive, ES, m/z): 379.27. ¹H NMR (400 MHz, DMSO) δ 8.99 (s, 1H), 8.79 (s, 1H), 8.72 (d, J=5.2 Hz, 1H), 8.42-8.32 (m, 1H), 7.85 (d, J=8.6 Hz, 1H), 7.18 (d, J=5.4 Hz, 1H), 6.86 (dd, J=16.7, 10.4 Hz, 1H), 6.16 (d, J=16.5 Hz, 1H), 5.73 (d, J=10.5 Hz, 1H), 3.78 (s, 4H), 3.06 (s, 4H).

### Example 122: Synthesis of 1-(4-(5-((4-(trifluoromethyl)phenyl)amino)pyridazin-4-yl)piperazin-1-yl)prop-2-en-1-one

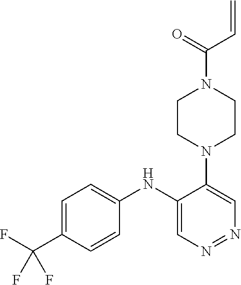 [SMILES: C=CC(=O)N1CCN(c2cnncc2Nc2ccc(C(F)(F)F)cc2)CC1]

Following the general procedure in scheme 3, 5-chloropyridazin-4-amine was used in step 1. Offered 1-(4-(5-((4-(trifluoromethyl)phenyl)amino)pyridazin-4-yl)piperazin-1-yl)prop-2-en-1-one as white solid. (Positive, ES, m/z): 378.28. ¹H NMR (400 MHz, DMSO) δ 8.85 (s, 1H), 8.79 (d, J=2.6 Hz, 2H), 7.63 (d, J=8.4 Hz, 2H), 7.11 (d, J=8.4 Hz, 2H), 6.79 (dd, J=16.6, 10.4 Hz, 1H), 6.12 (dd, J=16.7, 2.4 Hz, 1H), 5.69 (dd, J=10.4, 2.4 Hz, 1H), 3.44 (d, J=14.7 Hz, 4H), 3.08 (t, J=5.1 Hz, 4H).

### Example 123. Synthesis of 1-[4-(2-{[4-(trifluoromethyl)phenyl]amino}pyrimidin-4-yl)piperazin-1-yl]prop-2-en-1-one

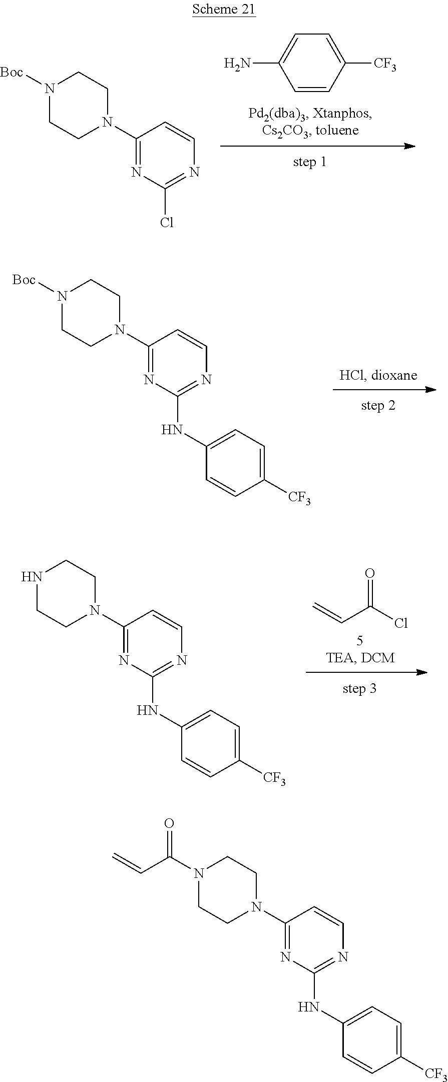

### Step 1: Synthesis of tert-butyl 4-(2-{[4-(trifluoromethyl)phenyl]amino}pyrimidin-4-yl)piperazine-1-carboxylate

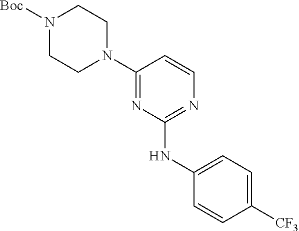 [SMILES: CC(C)(C)OC(=O)N1CCN(c2ccnc(Nc3ccc(C(F)(F)F)cc3)n2)CC1]

To a solution oftert-butyl 4-(2-chloropyrimidin-4-yl)piperazine-1-carboxylate (800.00 mg, 2.68 mmol, 1.00 equiv), p-trifluoromethylaniline (431.44 mg, 2.68 mmol, 1 equiv) and Cs₂CO₃ (1744.86 mg, 5.36 mmol, 2 equiv) in toluene (6.00 mL) was added Pd₂(dba)₃ (245.20 mg, 0.27 mmol, 0.1 equiv). The resulting mixture was stirred for 3 h at 90° C. under N₂ atmosphere. The reaction mixture was diluted with EA (90.00 mL). The organic layers were washed with H₂O (3×20.00 mL), dried over anhydrous Na₂SO₄. After filtration, the filtrate was concentrated under reduced pressure. The residue was purified by silica gel column chromatography, eluted with PE:EA (3:1) to afford tert-butyl 4-(2-{[4-(trifluoromethyl)phenyl]amino}pyrimidin-4-yl)piperazine-1-carboxylate (650.00 mg, 57.33%) as a yellow soild. LCMS (ES, m/z): [M+H]⁺=424

### Step 2: Synthesis of 4-(piperazin-1-yl)-N-[4-(trifluoromethyl)phenyl]pyrimidin-2-amine

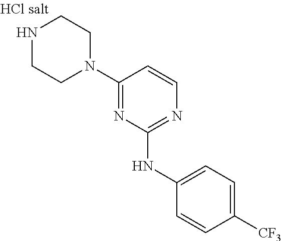 [SMILES: FC(F)(F)c1ccc(Nc2nccc(N3CCNCC3)n2)cc1]

Into a 40 mL vessel was placed a solution of tert-butyl 4-(2-{[4-(trifluoromethyl)phenyl]amino}pyrimidin-4-yl)piperazine-1-carboxylate (450.00 mg, 1.06 mmol, 1.00 equiv) in DCM (2.00 mL). HCl(gas) in 1,4-dioxane (2.6 mL, 10.6 mmol, 10.00 equiv) was added at 0° C. The resulting mixture was stirred for 1 h at room temperature. The mixture was concentrated under vacuum. This resulted in 4-(piperazin-1-yl)-N-[4-(trifluoromethyl)phenyl]pyrimidin-2-amine (300.00 mg, 87.31%) as a yellow solid. LCMS (ES, m/z): [M+H]⁺=324

### Step 3: Synthesis of 1-[4-(2-{[4-(trifluoromethyl)phenyl]amino}pyrimidin-4-yl)piperazin-1-yl]prop-2-en-1-one

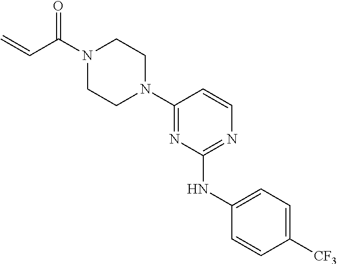 [SMILES: C=CC(=O)N1CCN(c2ccnc(Nc3ccc(C(F)(F)F)cc3)n2)CC1]

Into a 40 mL vessel was placed a solution of 4-(piperazin-1-yl)-N-[4-(trifluoromethyl)phenyl]pyrimidin-2-amine (300.00 mg, 0.93 mmol, 1.00 equiv) and TEA (281.68 mg, 2.78 mmol, 3.00 equiv) in DCM (5.00 mL). Acryloyl chloride (83.98 mg, 0.93 mmol, 1.00 equiv) was added at 0° C. The resulting mixture was stirred for 1 h at room temperature. The mixture was diluted with EA (45.00 mL), washed with H₂O (3×15.00 mL), dried over anhydrous Na₂SO₄. After filtration, the filtrate was concentrated under reduced pressure. The crude product (350.00 mg) was purified by Prep-HPLC with the following conditions (Column: Welch Xtimate C18 ExRS, 250 mm, 10 μm; Mobile Phase A: Water (0.05⁰% oNH₃·H₂O), Mobile Phase B: ACN; Flow rate: 50 mL/min; Gradient: 60% B to 90% B in 10 min, 90% B; Wave Length: 254 nm; RT1(min): 7; Number Of Runs: 5) to afford 1-[4-(2-{[4-(trifluoromethyl)phenyl]amino}pyrimidin-4-yl)piperazin-1-yl]prop-2-en-1-one (70.00 mg, 19.99%) as a white solid. LCMS (ES, m/z): [M+H]⁺=378. ¹H NMR (400 MHz, DMSO-d₆): δ 9.56 (s, 1H), 8.07 (s, 1H), 8.05 (d, J=14.0 Hz, 2H), 7.93 (d, J=12.OHz, 2H), 6.91-6.82 (m, 1H), 6.40 (s, 1H), 6.20 (s, 1H), 6.14 (s, 1H), 3.96-3.86 (m, 8H).

### Example 124: Synthesis of 1-(4-(methyl(2-((4-(trifluoromethyl)phenyl)amino)pyrimidin-4-yl)amino)piperidin-1-yl)prop-2-en-1-one

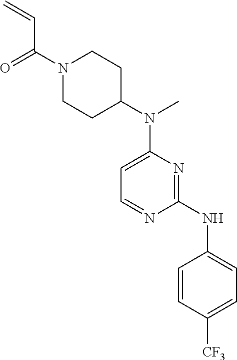 [SMILES: C=CC(=O)N1CCC(N(C)c2ccnc(Nc3ccc(C(F)(F)F)cc3)n2)CC1]

Following the general procedure in scheme 21, tert-butyl 4-(methylamino)piperidine-1-carboxylate was used in step 1. Offered 1-(4-(methyl(2-((4-(trifluoromethyl)phenyl)amino)pyrimidin-4-yl)amino)piperidin-1-yl)prop-2-en-1-one as white solid. (Positive, ES, m/z): 406.32. ¹H NMR (400 MHz, DMSO) δ 10.21 (s, 1H), 8.14-7.92 (m, 1H), 7.94-7.32 (m, 4H), 6.93-6.70 (m, 1H), 6.25-6.02 (m, 1H), 5.68 (ddd, J=10.5, 8.0, 2.5 Hz, 1H), 4.94-4.35 (m, 2H), 4.34-3.96 (m, 1H), 3.26-3.05 (m, 1H), 3.07-2.78 (m, 3H), 2.72 (t, J=12.9 Hz, 1H), 1.71 (s, 4H).

### Scheme 22. Synthesis of 1-[4-(4-{[4-(trifluoromethyl)phenyl]amino}pyrimidin-2-yl)piperazin-1-yl]prop-2-en-1-one (Example 125)

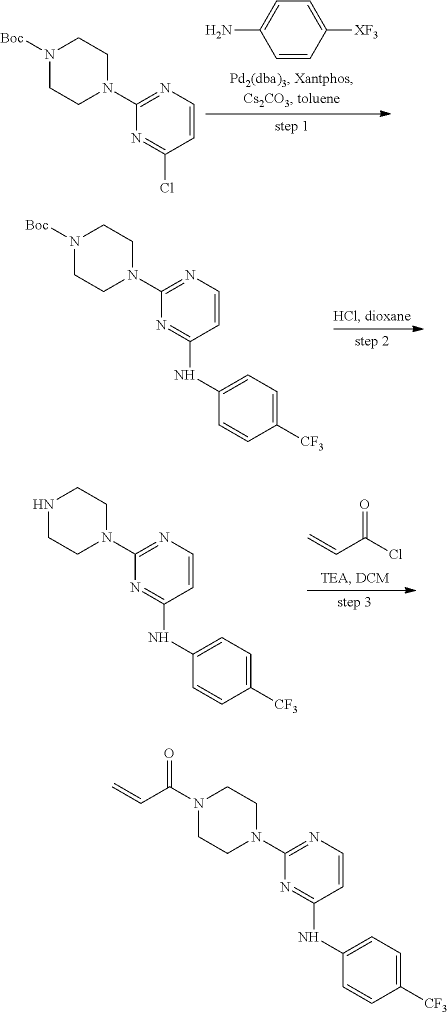

### Step 1: Synthesis of tert-butyl 4-(4-{[4-(trifluoromethyl)phenyl]amino}pyrimidin-2-yl)piperazine-1-carboxylate

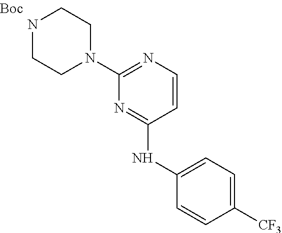 [SMILES: FC(F)(F)c1ccc(Nc2ccnc(N3CCNCC3)n2)cc1]

To a solution of tert-butyl 4-(4-chloropyrimidin-2-yl)piperazine-1-carboxylate (650.00 mg, 2.18 mmol, 1.00 equiv),p-trifluoromethylaniline (350.55 mg, 2.18 mmol, 1.00 equiv) and Cs₂CO₃ (1417.69 mg, 4.35 mmol, 2.00 equiv) in toluene (5.00 mL) was added Pd₂(dba)₃ (199.22 mg, 0.22 mmol, 0.10 equiv). The resulting mixture was stirred for 3 h at 90° C. under N2 atmosphere. The mixture was diluted with EA (75.00 mL), washed with H₂O (3×25.00 mL), dried over anhydrous Na₂SO₄. After filtration, the filtrate was concentrated under reduced pressure. The residue was purified by silica gel column chromatography, eluted with PE:EA (3:1) to afford tert-butyl 4-(4-{[4-(trifluoromethyl)phenyl]amino}pyrimidin-2-yl)piperazine-1-carboxylate (600.00 mg, 65.13%) as a yellow solid. LCMS (ES, m/z): [M+H]⁺=424

### Step 2: Synthesis of 2-(piperazin-1-yl)-N-[4-(trifluoromethyl)phenyl]pyrimidin-4-amine

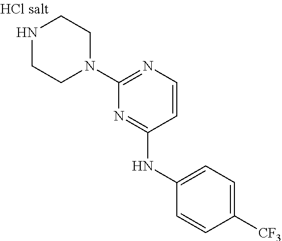 [SMILES: FC(F)(F)c1ccc(Nc2ccnc(N3CCNCC3)n2)cc1]

Into a 40 mL vessel was placed a solution of tert-butyl 4-(4-{[4-(trifluoromethyl)phenyl]amino}pyrimidin-2-yl)piperazine-1-carboxylate (450.00 mg, 1.06 mmol, 1.00 equiv) in DCM (2.00 mL). HCl(gas) in 1,4-dioxane (2.6 mL, 10.6 mmol, 10.00 equiv) was added at 0° C. The resulting mixture was stirred for 1 h at room temperature. The mixture was concentrated under vacuum. This resulted in 2-(piperazin-1-yl)-N-[4-(trifluoromethyl)phenyl]pyrimidin-4-amine (300.00 mg, 87.31%) as a yellow solid. LCMS (ES, m/z):[M+H]⁺=324.

### Step 3: Synthesis of 1-[4-(4-{[4-(trifluoromethyl)phenyl]amino}pyrimidin-2-yl)piperazin-1-yl]prop-2-en-1-one

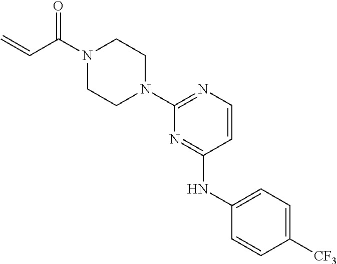 [SMILES: C=CC(=O)N1CCN(c2nccc(Nc3ccc(C(F)(F)F)cc3)n2)CC1]

Into a 40 mL vessel were placed a solution of 2-(piperazin-1-yl)-N-[4-(trifluoromethyl)phenyl]pyrimidin-4-amine (300.00 mg, 0.93 mmol, 1.00 equiv) and TEA (281.68 mg, 2.78 mmol, 3.00 equiv) in DCM (5.00 mL). Acryloyl chloride (83.98 mg, 0.93 mmol, 1.00 equiv) was added at 0° C. The resulting mixture was stirred for 1 h at room temperature. The mixture was diluted with EA (45.00 mL), washed with H₂O (3×15.00 mL), dried over anhydrous Na₂SO₄. After filtration, the filtrate was concentrated under reduced pressure. The crude product (350.00 mg) was purified by Prep-HPLC with the following conditions (Column: Welch Xtimate C18 ExRS, 250 mm, 10 μm; Mobile Phase A: Water (0.05⁰% NH₃·H₂O), Mobile Phase B: ACN; Flow rate: 90 mL/min; Gradient: 90% B to 90% B in 10 min, 90% B; Wave Length: 254 nm; RT1(min): 7; Number Of Runs: 5) to afford 1-[4-(4-{[4-(trifluoromethyl)phenyl]amino}pyrimidin-2-yl)piperazin-1-yl]prop-2-en-1-one (70.00 mg, 19.99%) as a white solid. LCMS (ES, m/z): [M+H]⁺=378 ¹H NMR (300 MHz, DMSO-d₆): δ 9.7 (s, 1H), 8.05 (s, 1H), 7.83 (d, J=2.9 Hz, 2H), 7.69 (d, J=3.5 Hz, 2H), 6.90-6.80 (m, 1H), 6.40-6.38 (m, 1H), 6.19-6.13 (m, 1H), 5.75-5.70 (m, 1H), 3.75-3.65 (m, 8H).

### Example 126: Synthesis of 1-(4-(4-((6-(trifluoromethyl)pyridin-3-yl)oxy)pyrimidin-5-yl)piperidin-1-yl)prop-2-en-1-one

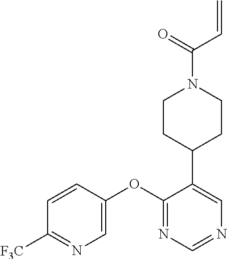 [SMILES: C=CC(=O)N1CCC(c2cncnc2Oc2ccc(C(F)(F)F)nc2)CC1]

Following the general procedure in scheme 7, tert-butyl 4-(4-((6-(trifluoromethyl)pyridin-3-yl)oxy)pyrimidin-5-yl)piperidine-1-carboxylate was used in step 1. Offered 1-(4-(4-((6-(trifluoromethyl)pyridin-3-yl)oxy)pyrimidin-5-yl)piperidin-1-yl)prop-2-en-1-one as white solid. (Positive, ES, m/z):379.39. ¹H NMR (400 MHz, DMSO) δ 8.90-8.55 (m, 3H), 8.08 (d, J=1.6 Hz, 2H), 6.87 (dd, J=16.7, 10.5 Hz, 1H), 6.13 (dd, J=16.7, 2.5 Hz, 1H), 5.70 (dd, J=10.5, 2.5 Hz, 1H), 4.64 (d, J=13.1 Hz, 1H), 4.24 (d, J=13.8 Hz, 1H), 3.29-3.12 (m, 2H), 2.78 (t, J=12.7 Hz, 1H), 2.01 (d, J=12.8 Hz, 2H), 1.88-1.60 (m, 2H).

### Example 127: Synthesis of 2-fluoro-1-(4-(4-((6-(trifluoromethyl)pyridin-3-yl)oxy)pyrimidin-5-yl)piperidin-1-yl)prop-2-en-1-one

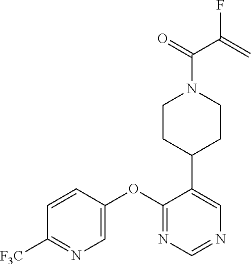 [SMILES: C=C(F)C(=O)N1CCC(c2cncnc2Oc2ccc(C(F)(F)F)nc2)CC1]

Following the general procedure in scheme 10, 5-(piperidin-4-yl)-4-((6-(trifluoromethyl)pyridin-3-yl)oxy)pyrimidine was used. Offered 2-fluoro-1-(4-(4-((6-(trifluoromethyl)pyridin-3-yl)oxy)pyrimidin-5-yl)piperidin-1-yl)prop-2-en-1-one as white solid. (Positive, ES, m/z):397.79. ¹H NMR (400 MHz, DMSO) δ 8.79 (s, 1H), 8.73 (s, 1H), 8.65 (s, 1H), 8.08 (d, J=1.6 Hz, 2H), 5.36-5.10 (m, 2H), 4.47 (s, 1H), 4.07 (s, 1H), 3.29 (ddd, J=12.6, 8.7, 3.6 Hz, 2H), 2.92 (s, 1H), 2.13-1.98 (m, 2H), 1.91-1.69 (m, 2H).

### Example 128: Synthesis of 1-(4-(2-methyl-5-((4-(trifluoromethyl)phenyl)amino)pyrimidin-4-yl)piperazin-1-yl)prop-2-en-1-one

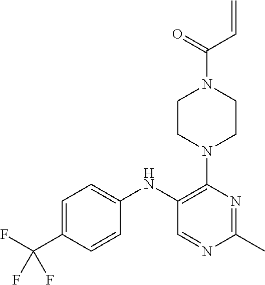 [SMILES: C=CC(=O)N1CCN(c2nc(C)ncc2Nc2ccc(C(F)(F)F)cc2)CC1]

Following the general procedure in scheme 3, 4-chloro-2-methylpyrimidin-5-amine was used in step 1. Offered 1-(4-(2-methyl-5-((4-(trifluoromethyl)phenyl)amino)pyrimidin-4-yl)piperazin-1-yl)prop-2-en-1-one as white solid. (Positive, ES, m/z): 392.19. ¹H NMR (400 MHz, DMSO) δ 8.30 (s, 1H), 8.08 (s, 1H), 7.48 (d, J=8.5 Hz, 2H), 6.77 (dd, J=16.7, 10.4 Hz, 1H), 6.69 (d, J=8.5 Hz, 2H), 6.10 (dd, J=16.7, 2.4 Hz, 1H), 5.67 (dd, J=10.4, 2.4 Hz, 1H), 3.59 (t, J=4.8 Hz, 4H), 3.52-3.39 (m, 4H), 2.46 (s, 3H).

### Example 129: Synthesis of 1-(4-(2-methyl-4-((6-(trifluoromethyl)pyridin-3-yl)oxy)pyrimidin-5-yl)-3,6-dihydropyridin-1(2H)-yl)prop-2-en-1-one

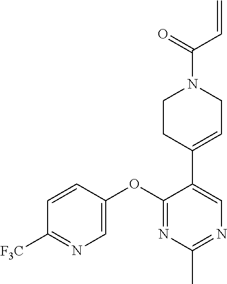 [SMILES: C=CC(=O)N1CC=C(c2cnc(C)nc2Oc2ccc(C(F)(F)F)nc2)CC1]

Following the general procedure in scheme 6, 5-chloro-2-methyl-4-((6-(trifluoromethyl)pyridin-3-yl)oxy)pyrimidine was used in step 1. Offered 1-(4-(2-methyl-4-((6-(trifluoromethyl)pyridin-3-yl)oxy)pyrimidin-5-yl)-3,6-dihydropyridin-1(2H)-yl)prop-2-en-1-one as white solid. (Positive, ES, m/z): 391.27. ¹H NMR (400 MHz, DMSO) δ 8.78 (d, J=1.7 Hz, 1H), 8.60 (d, J=2.2 Hz, 1H), 8.06 (d, J=1.6 Hz, 2H), 6.95-6.75 (m, 1H), 6.27 (s, 1H), 6.16 (d, J=16.4 Hz, 1H), 5.72 (d, J=10.5 Hz, 1H), 4.38-4.17 (m, 2H), 3.77 (dt, J=12.0, 5.7 Hz, 2H), 2.71-2.55 (m, 2H), 2.42 (s, 3H).

### Example 130: Synthesis of 2-fluoro-1-(4-(2-methyl-4-((6-(trifluoromethyl)pyridin-3-yl)oxy)pyrimidin-5-yl)-3, 6-dihydropyridin-1(2H)-yl)prop-2-en-1-one

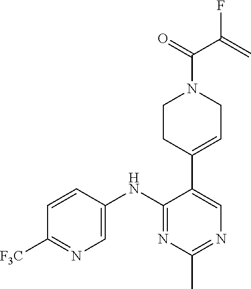 [SMILES: C=C(F)C(=O)N1CC=C(c2cnc(C)nc2Nc2ccc(C(F)(F)F)nc2)CC1]

Following the general procedure in scheme 10, 2-methyl-5-(1,2,3,6-tetrahydropyridin-4-yl)-4-((6-(trifluoromethyl)pyridin-3-yl)oxy)pyrimidine was used. Offered 2-fluoro-1-(4-(2-methyl-4-((6-(trifluoromethyl)pyridin-3-yl)oxy)pyrimidin-5-yl)-3, 6-dihydropyridin-1(2H)-yl)prop-2-en-1-one as white solid. (Positive, ES, m/z):409.19. ¹H NMR (400 MHz, DMSO) δ 8.78 (t, J=1.7 Hz, IH), 8.61 (s, IH), 8.06 (d, J=1.6 Hz, 2H), 6.27 (s, 1H), 5.40-5.11 (m, 2H), 4.35-4.11 (m, 2H), 3.75 (t, J=5.6 Hz, 2H), 2.66 (s, 2H), 2.43 (s, 3H).

### Example 131: Synthesis of 1-(4-(2-methyl-4-((6-(trifluoromethyl)pyridin-3-yl)oxy)pyrimidin-5-yl)piperidin-1-yl)prop-2-en-1-one

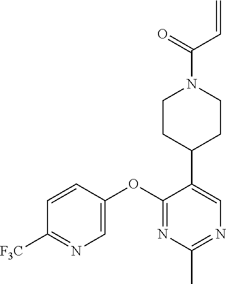 [SMILES: C=CC(=O)N1CCC(c2cnc(C)nc2Oc2ccc(C(F)(F)F)nc2)CC1]

Following the general procedure in scheme 7, tert-butyl 4-(2-methyl-4-((6-(trifluoromethyl)pyridin-3-yl)oxy)pyrimidin-5-yl)piperidine-1-carboxylate was used in step 1. Offered 1-(4-(2-methyl-4-((6-(trifluoromethyl)pyridin-3-yl)oxy)pyrimidin-5-yl)piperidin-1-yl)prop-2-en-1-one as white solid. (Positive, ES, m/z): 391.27. ¹H NMR (400 MHz, DMSO) δ 8.78 (d, J=1.7 Hz, 1H), 8.60 (d, J=2.2 Hz, 1H), 8.06 (d, J=1.6 Hz, 2H), 6.95-6.75 (m, 1H), 6.27 (s, 1H), 6.16 (d, J=16.4 Hz, 1H), 5.72 (d, J=10.5 Hz, 1H), 4.38-4.17 (m, 2H), 3.77 (dt, J=12.0, 5.7 Hz, 2H), 2.71-2.55 (m, 2H), 2.42 (s, 3H).

### Example 132: Synthesis of 2-fluoro-1-(4-(2-methyl-4-((6-(trifluoromethyl)pyridin-3-yl)oxy)pyrimidin-5-yl)piperidin-1-yl)prop-2-en-1-one

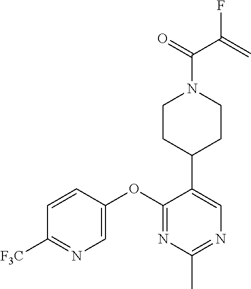 [SMILES: C=C(F)C(=O)N1CCC(c2cnc(C)nc2Oc2ccc(C(F)(F)F)nc2)CC1]

Following the general procedure in scheme 10, 2-methyl-5-(piperidin-4-yl)-4-((6-(trifluoromethyl)pyridin-3-yl)oxy)pyrimidine was used. Offered 2-fluoro-1-(4-(2-methyl-4-((6-(trifluoromethyl)pyridin-3-yl)oxy)pyrimidin-5-yl)piperidin-1-yl)prop-2-en-1-one as white solid. (Positive, ES, m/z):411.2. ¹H NMR (400 MHz, DMSO) δ 8.82-8.58 (m, 1H), 8.32-7.99 (m, 2H), 7.81-7.34 (m, 1H), 5.35-5.08 (m, 2H), 4.45 (s, 1H), 3.23 (t, J=12.3 Hz, 2H), 2.88 (s, 1H), 2.43-2.31 (m, 3H), 2.01 (d, J=12.8 Hz, 2H), 1.86-1.66 (m, 2H).

### Example 133: Synthesis of 1-(4-(2-methoxy-5-((4-(trifluoromethyl)phenyl)amino)pyrimidin-4-yl)piperazin-1-yl)prop-2-en-1-one

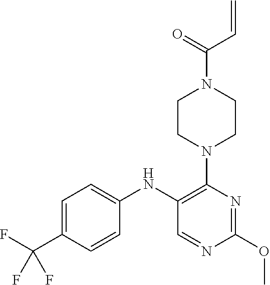 [SMILES: C=CC(=O)N1CCN(c2nc(OC)ncc2Nc2ccc(C(F)(F)F)cc2)CC1]

Following the general procedure in scheme 3, 4-chloro-2-methoxypyrimidin-5-amine was used in step 1. Offered 1-(4-(2-methoxy-5-((4-(trifluoromethyl)phenyl)amino)pyrimidin-4-yl)piperazin-1-yl)prop-2-en-1-one as white solid. (Positive, ES, m/z): 393.2. ¹H NMR (400 MHz, DMSO) δ 8.80-8.56 (m, 1H), 8.35-8.04 (m, 2H), 7.82-7.68 (m, 2H), 7.36 (dd, J=8.5, 2.8 Hz, 1H), 6.91-6.74 (m, 1H), 6.12 (ddd, J=16.7, 5.3, 2.5 Hz, 1H), 5.69 (ddd, J=10.4, 4.4, 2.4 Hz, 1H), 4.57 (d, J=13.1 Hz, 1H), 4.27-4.16 (m, 1H), 3.22-3.06 (m, 2H), 2.84 (t, J=12.4 Hz, 1H), 2.42-2.31 (m, 4H), 1.96 (s, 2H), 1.49 (d, J=14.0 Hz, 2H).

### Example 134. Synthesis of 1-[4-(3-{[4-(trifluoromethyl)phenyl]amino}pyridin-2-yl)piperazin-1-yl]prop-2-en-1-one

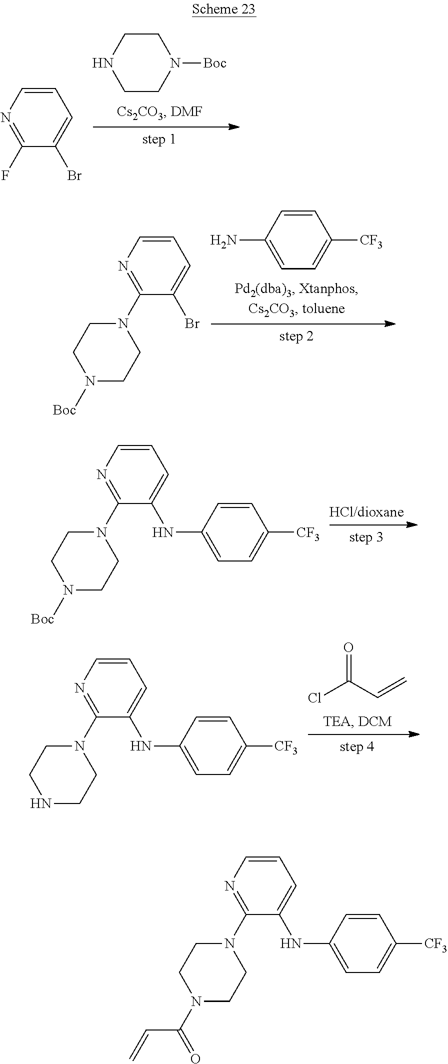

### Step 1: Synthesis of tert-butyl 4-(3-bromopyridin-2-yl)piperazine-1-carboxylate

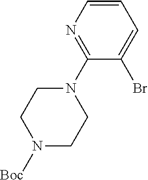 [SMILES: CC(C)(C)OC(=O)N1CCN(c2ncccc2Br)CC1]

Into a 40 mL vessel was placed a solution of 3-bromo-2-fluoropyridine (1.00 g, 5.68 mmol, 1.00 equiv), tert-butyl piperazine-1-carboxylate (1.06 g, 5.68 mmol, 1.00 equiv) and Cs₂CO₃ (3.70 g, 11.36 mmol, 2.00 equiv) in DMF (6.00 mL). The mixture was stirred overnight at 80° C. The mixture was diluted with EA (60.00 mL). The organic layers were washed with H₂O (3×20.00 mL), dried over anhydrous Na₂SO₄. After filtration, the filtrate was concentrated under reduced pressure. The residue was purified by silica gel column chromatography, eluted with PE:EA (2:1) to afford tert-butyl 4-(3-bromopyridin-2-yl)piperazine-1-carboxylate (850.00 mg, 43.71%) as a yellow solid. LCMS (ES, m/z): [M+H]⁺=342

### Step 2: Synthesis of tert-butyl 4-(3-{[4-(trifluoromethyl)phenyl]amino}pyridin-2-yl)piperazine-1-carboxylate

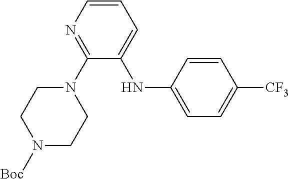 [SMILES: CC(C)(C)OC(=O)N1CCN(c2ncccc2Nc2ccc(C(F)(F)F)cc2)CC1]

A solution of tert-butyl 4-(3-bromopyridin-2-yl)piperazine-1-carboxylate (830.00 mg, 2.43 mmol, 1.00 equiv), p-trifluoromethylaniline (390.77 mg, 2.43 mmol, 1.00 equiv), Cs₂CO₃ (1850.37 mg, 4.85 mmol, 2.00 equiv) and Pd₂(dba)₃ (222.08 mg, 0.24 mmol, 0.1 equiv) in toluene (5.00 mL) was stirred for 3 h at 90° C. under N₂ atmosphere. The mixture was diluted with EA (75.00 mL), washed with H₂O (3×25.00 mL), dried over anhydrous Na₂SO₄. After filtration, the filtrate was concentrated under reduced pressure. The residue was purified by silica gel column chromatography, eluted with PE:EA (2:1) to afford tert-butyl 4-(3-{[4-(trifluoromethyl)phenyl]amino}pyridin-2-yl)piperazine-1-carboxylate (640.00 mg, 62.47%) as a yellow solid. LCMS (ES, m/z): [M+H]-=423

### Step 3: Synthesis of 2-(piperazin-1-yl)-N-[4-(trifluoromethyl)phenyl]pyridin-3-amine

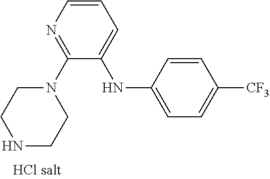 [SMILES: FC(F)(F)c1ccc(Nc2cccnc2N2CCNCC2)cc1]

Into a 40 mL vessel was placed a solution of tert-butyl 4-(3-{[4-(trifluoromethyl)phenyl]amino}pyridin-2-yl)piperazine-1-carboxylate (450.00 mg, 1.07 mmol, 1.00 equiv) in DCM (2.00 mL). HCl(gas) in 1,4-dioxane (2.6 mL, 10.6 mmol, 10.00 equiv) was added at 0° C. The resulting mixture was stirred for 1 h at room temperature. The mixture was concentrated under vacuum. This resulted in 2-(piperazin-1-yl)-N-[4-(trifluoromethyl)phenyl]pyridin-3-amine (300.00 mg, 87.37%) as a yellow solid. LCMS (ES, m/z): [M+H]⁺=323

### Step 4: Synthesis of 1-[4-(3-{[4-(trifluoromethyl)phenyl]amino}pyridin-2-yl)piperazin-1-yl]prop-2-en-1-one

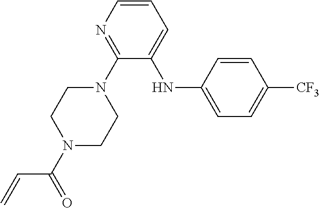 [SMILES: C=CC(=O)N1CCN(c2ncccc2Nc2ccc(C(F)(F)F)cc2)CC1]

Into a 40 mL vial was placed a solution of 2-(piperazin-1-yl)-N-[4-(trifluoromethyl)phenyl]pyridin-3-amine (350.00 mg, 1.09 mmol, 1.00 equiv) and TEA (329.63 mg, 3.26 mmol, 3.00 equiv) in DCM (5.00 mL). Acryloyl chloride (98.28 mg, 1.09 mmol, 1.00 equiv) was added at 0° C. The mixture was stirred for 1 h at room temperature. After the reaction was completed, it was diluted with EA (45.00 mL), washed with H₂O (3×15.00 mL), dried over anhydrous Na₂SO₄. After filtration, the filtrate was concentrated under reduced pressure. The crude product (200.00 mg) was purified by Prep-HPLC with the following conditions (Column: Welch Xtimate C18 ExRS, 250 mm, 10 μm; Mobile Phase A: Water (0.05% NH₃·H₂O), Mobile Phase B: ACN; Flow rate: 90 mL/min; Gradient: 90% B to 90% B in 10 min, 90% B; Wave Length: 254 nm; RT1(min): 7; Number Of Runs: 5) to afford 1-[4-(3-{[4-(trifluoromethyl)phenyl]amino}pyridin-2-yl)piperazin-1-yl]prop-2-en-1-one (110 mg, 26.92%) as a white solid. LCMS (ES, m/z): [M+H]⁺=377. ¹H NMR (400 MHz, DMSO-d₆): δ 8.15 (s, 1H), 8.04-8.02 (m, 1H), 7.59-7.58 (m, 1H), 7.56-7.52 (m, 2H), 7.01-6.97 (m, 3H), 6.84-6.75 (m, 1H), 6.13-6.07 (m, 1H), 6.19-6.15 (m, 1H), 5.69-5.65 (m, 1H), 3.57-3.51 (m, 4H), 3.17-3.13 (m, 4H).

### Example 135: Synthesis of 2-fluoro-1-(4-(3-((6-(trifluoromethyl)pyridin-3-yl)amino)pyridin-2-yl)piperazin-1-yl)prop-2-en-1-one

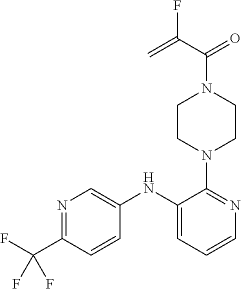 [SMILES: C=C(F)C(=O)N1CCN(c2ncccc2Nc2ccc(C(F)(F)F)nc2)CC1]

Following the general procedure in scheme 10, 2-(piperazin-1-yl)-N-(6-(trifluoromethyl)pyridin-3-yl)pyridin-3-amine was used. Offered 2-fluoro-1-(4-(3-((6-(trifluoromethyl)pyridin-3-yl)amino)pyridin-2-yl)piperazin-1-yl)prop-2-en-1-one as white solid. (Positive, ES, m/z):396.1. ¹H NMR (400 MHz, DMSO) δ 8.53 (s, 1H), 8.31 (d, J=2.7 Hz, 1H), 8.09 (dd, J=4.8, 1.6 Hz, 1H), 7.71-7.56 (m, 2H), 7.24 (dd, J=8.6, 2.7 Hz, 1H), 7.04 (dd, J=7.8, 4.7 Hz, 1H), 5.31-5.05 (m, 2H), 3.47 (t, J=5.0 Hz, 4H), 3.19 (d, J=5.3 Hz, 4H).

### Example 136: Synthesis of 1-(3-((6-(trifluoromethyl)pyridin-3-yl)oxy)-3′,6′-dihydro-[2,4′-bipyridin]-1′(2′H)-yl)prop-2-en-1-one

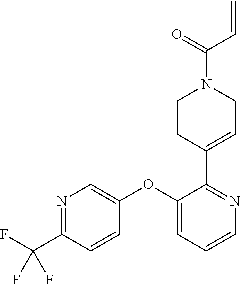 [SMILES: C=CC(=O)N1CC=C(c2ncccc2Oc2ccc(C(F)(F)F)nc2)CC1]

Following the general procedure in scheme 6, 2-chloro-3-((6-(trifluoromethyl)pyridin-3-yl)oxy)pyridine was used in step 1. Offered 1-(3-((6-(trifluoromethyl)pyridin-3-yl)oxy)-3′,6′-dihydro-[2,4′-bipyridin]-1′(2′H)-yl)prop-2-en-1-one as white solid. (Positive, ES, m/z):409.1. ¹H NMR (400 MHz, DMSO) δ 8.52 (d, J=2.8 Hz, 1H), 8.26-8.16 (m, 2H), 8.09 (dd, J=4.9, 1.8 Hz, 1H), 7.50 (dd, J=7.4, 1.9 Hz, 1H), 7.22 (d, J=8.8 Hz, 1H), 6.91 (dd, J=7.4, 4.9 Hz, 1H), 5.91 (s, 1H), 5.41-5.13 (m, 2H), 4.31-4.08 (m, 2H), 3.76 (d, J=5.7 Hz, 2H), 3.41 (d, J=1.0 Hz, 2H), 2.47 (s, 2H).

### Example 137: Synthesis of2-fluoro-1-(3-((6-(trifluoromethyl)pyridin-3-yl)oxy)-3′,6′-dihydro-[2,4′-bipyridin]-1′(2′H)-yl)prop-2-en-1-one

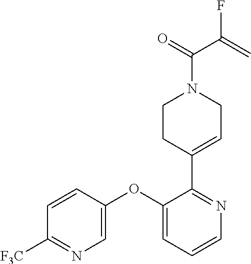 [SMILES: C=C(F)C(=O)N1CC=C(c2ncccc2Oc2ccc(C(F)(F)F)nc2)CC1]

Following the general procedure in scheme 10, 3-((6-(trifluoromethyl)pyridin-3-yl)oxy)-1′,2′,3′,6′-tetrahydro-2,4′-bipyridine was used. Offered 2-fluoro-1-(3-((6-(trifluoromethyl)pyridin-3-yl)oxy)-3′,6′-dihydro-[2,4′-bipyridin]-1′(2′H)-yl)prop-2-en-1-one as white solid. (Positive, ES, m/z):394.1. ¹H NMR (400 MHz, DMSO) δ 8.54 (dd, J=13.7, 3.8 Hz, 2H), 7.89 (d, J=8.8 Hz, 1H), 7.71 (dd, J=15.8, 8.3 Hz, 1H), 7.53 (s, 1H), 7.43 (dd, J=8.3, 4.5 Hz, 1H), 6.46 (s, 1H), 5.36-5.06 (m, 2H), 4.10 (s, 2H), 3.63 (t, J=5.7 Hz, 2H), 2.68 (s, 2H).

### Example 138: Synthesis of 1-(4-(3-((6-(trifluoromethyl)pyridin-3-yl)oxy)pyridin-2-yl)piperidin-1-yl)prop-2-en-1-one

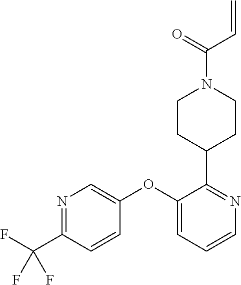 [SMILES: C=CC(=O)N1CCC(c2ncccc2Oc2ccc(C(F)(F)F)nc2)CC1]

Following the general procedure in scheme 7, tert-butyl 3-((6-(trifluoromethyl)pyridin-3-yl)oxy)-3′,6′-dihydro-[2,4′-bipyridine]-1′(2′H)-carboxylate was used in step 1. Offered 1-(4-(3-((6-(trifluoromethyl)pyridin-3-yl)oxy)pyridin-2-yl)piperidin-1-yl)prop-2-en-1-one as white solid. (Positive, ES, m/z):378.1. ¹H NMR (400 MHz, DMSO) δ 8.63 (d, J=2.8 Hz, 1H), 8.47 (dd, J=4.6, 1.4 Hz, 1H), 7.92 (d, J=8.7 Hz, 1H), 7.58 (td, J=9.1, 2.1 Hz, 2H), 7.37 (dd, J=8.2, 4.6 Hz, 1H), 6.84 (dd, J=16.7, 10.4 Hz, 1H), 6.11 (dd, J=16.7, 2.5 Hz, 1H), 5.67 (dd, J=10.5, 2.4 Hz, 1H), 4.52 (d, J=13.2 Hz, 1H), 4.14 (d, J=13.7 Hz, 1H), 3.32-3.27 (m, 1H), 3.13 (d, J=15.4 Hz, 1H), 2.78-2.68 (m, 1H), 1.86-1.64 (m, 4H).

### Example 139: Synthesis of 2-fluoro-1-(4-(3-((6-(trifluoromethyl)pyridin-3-yl)oxy)pyridin-2-yl)piperidin-1-yl)prop-2-en-1-one

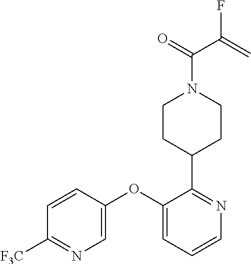 [SMILES: C=C(F)C(=O)N1CCC(c2ncccc2Oc2ccc(C(F)(F)F)nc2)CC1]

Following the general procedure in scheme 10, 2-(piperidin-4-yl)-3-((6-(trifluoromethyl)pyridin-3-yl)oxy)pyridine was used. Offered 2-fluoro-1-(4-(3-((6-(trifluoromethyl)pyridin-3-yl)oxy)pyridin-2-yl)piperidin-1-yl)prop-2-en-1-one as white solid. (Positive, ES, m/z):396.1. ¹H NMR (400 MHz, DMSO) δ 8.63 (d, J=2.8 Hz, 1H), 8.55-8.40 (m, 1H), 7.92 (d, J=8.8 Hz, 1H), 7.62-7.49 (m, 2H), 7.37 (dd, J=8.2, 4.6 Hz, 1H), 5.33-5.09 (m, 2H), 4.36 (s, 1H), 3.97 (s, 1H), 3.27 (s, 1H), 3.10-2.78 (m, 2H), 1.92-1.67 (m, 4H).

### Example 140: Synthesis of 1-(4-(3-(5-(trifluoromethyl)picolinoyl)pyridin-2-yl)piperazin-1-yl)prop-2-en-1-one

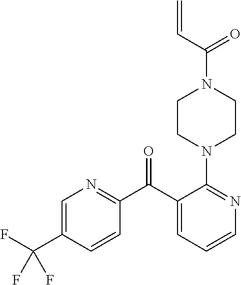 [SMILES: C=CC(=O)N1CCN(c2ncccc2C(=O)c2ccc(C(F)(F)F)cn2)CC1]

Following the general procedure in scheme 15, 2-bromopyridine and 5-(trifluoromethyl)nicotinaldehyde was used in step 1. Offered 1-(4-(3-(5-(trifluoromethyl)nicotinoyl)pyridin-2-yl)piperazin-1-yl)prop-2-en-1-one as white solid. (Positive, ES, m/z):391.27. ¹H NMR (400 MHz, DMSO) δ 9.08 (d, J=2.2 Hz, 1H), 8.51 (dd, J=8.3, 2.3 Hz, 1H), 8.40 (dd, J=4.7, 1.9 Hz, 1H), 8.22 (d, J=8.2 Hz, 1H), 7.85 (dd, J=7.6, 1.9 Hz, 1H), 6.99 (dd, J=7.6, 4.7 Hz, 1H), 6.72 (dd, J=16.7, 10.4 Hz, 1H), 6.09 (dd, J=16.7, 2.4 Hz, 1H), 5.67 (dd, J=10.4, 2.4 Hz, 1H), 3.39 (s, 4H), 3.22-3.15 (m, 4H).

### Example 141: Synthesis of 1-(4-(3-(6-(trifluoromethyl)nicotinoyl)pyridin-2-yl)piperazin-1-yl)prop-2-en-1-one

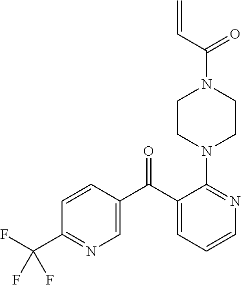 [SMILES: C=CC(=O)N1CCN(c2ncccc2C(=O)c2ccc(C(F)(F)F)nc2)CC1]

Following the general procedure in scheme 15, 2-bromopyridine and 6-(trifluoromethyl)nicotinaldehyde was used in step 1. Offered 1-(4-(3-(6-(trifluoromethyl)nicotinoyl)pyridin-2-yl)piperazin-1-yl)prop-2-en-1-one as white solid. (Positive, ES, m/z):391.00. ¹H NMR (400 MHz, DMSO) δ 9.06 (d, J=2.1 Hz, 1H), 8.46 (dd, J=4.7, 1.9 Hz, 1H), 8.39 (dd, J=8.2, 2.1 Hz, 1H), 8.10 (d, J=8.1 Hz, 1H), 7.88 (dd, J=7.6, 1.9 Hz, 1H), 7.05 (dd, J=7.6, 4.7 Hz, 1H), 6.72 (dd, J=16.7, 10.5 Hz, 1H), 6.09 (dd, J=16.7, 2.3 Hz, 1H), 5.67 (dd, J=10.4, 2.4 Hz, 1H), 3.34-3.26 (m, 4H), 3.21 (s, 4H).

### Example 142: Synthesis of 1-(4-(3-(hydroxy(6-(trifluoromethyl)pyridin-3-yl)methyl)pyridin-2-yl)piperazin-1-yl)prop-2-en-1-one

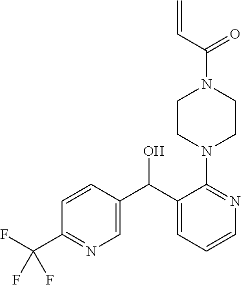 [SMILES: C=CC(=O)N1CCN(c2ncccc2C(O)c2ccc(C(F)(F)F)nc2)CC1]

Following the general procedure in scheme 16, tert-butyl 4-(3-(hydroxy(6-(trifluoromethyl)pyridin-3-yl)methyl)pyridin-2-yl)piperazine-1-carboxylate was used in step 1. Offered 1-(4-(3-(hydroxy(6-(trifluoromethyl)pyridin-3-yl)methyl)pyridin-2-yl)piperazin-1-yl)prop-2-en-1-one as white solid. (Positive, ES, m/z):393.29. ¹H NMR (400 MHz, DMSO) δ 8.78 (s, 1H), 8.28 (dd, J=4.8, 1.9 Hz, 1H), 7.98 (d, J=8.1 Hz, 1H), 7.91-7.78 (m, 2H), 7.19 (dd, J=7.6, 4.7 Hz, 1H), 6.85 (dd, J=16.7, 10.4 Hz, 1H), 6.38 (s, 1H), 6.27-6.09 (m, 2H), 5.71 (dd, J=10.5, 2.4 Hz, 1H), 3.86-3.54 (m, 4H), 3.20-3.06 (m, 2H), 2.80 (s, 2H).

### Example 143. Scheme 24. Synthesis of 1-[4-(2-{[4-(trifluoromethyl)phenyl]amino}pyridin-3-yl)piperazin-1-yl]prop-2-en-1-one

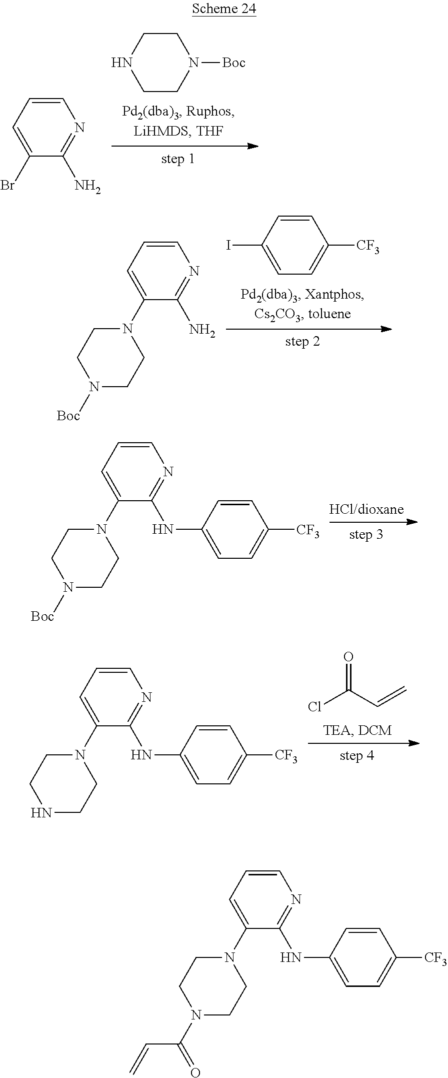

### Step 1: Synthesis of tert-butyl 4-(2-aminopyridin-3-yl)piperazine-1-carboxylate

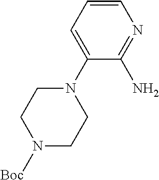 [SMILES: CC(C)(C)OC(=O)N1CCN(c2cccnc2N)CC1]

To a three-neck flask was placed a solution of 3-bromopyridin-2-amine (1.50 g, 8.67 mmol, 1.00 equiv), Pd₂(dba)₃ (0.32 g, 0.35 mmol, 0.04 equiv) and Ruphos (0.16 g, 0.35 mmol, 0.04 equiv) in THF (40.00 mL) under nitrogen atmosphere at room temperature. Tert-butyl piperazine-1-carboxylate (2.42 g, 13.01 mmol, 1.50 equiv) and LiHMDS (21.67 mL, 21.68 mmol, 2.50 equiv) was added to the reaction mixture. The resulting mixture was stirred for 12 h at room temperature under nitrogen atmosphere. Desired product was detected by LCMS. The mixture was quenched by the addition of saturated NH₄Cl solution (150.00 mL) and then extracted with EA (3×100.00 mL). The combined organic layers were washed with brine (2×100.00 mL), dried over anhydrous Na₂SO₄. After filtration, the filtrate was concentrated under reduced pressure and the residue was purified by silica gel column chromatography, eluted with DCM:EA (1:1) to afford tert-butyl 4-(2-aminopyridin-3-yl)piperazine-1-carboxylate (0.71 g, 29.42%) as a yellow solid. LCMS (Positive, ES, m/z): 279.2

### Step 2: Synthesis oftert-butyl 4-(2-{[4-(trifluoromethyl)phenyl]amino}pyridin-3-yl)piperazine-1-carboxylate

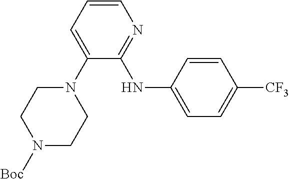 [SMILES: CC(C)(C)OC(=O)N1CCN(c2cccnc2Nc2ccc(C(F)(F)F)cc2)CC1]

To a stirred mixture oftert-butyl 4-(2-aminopyridin-3-yl)piperazine-1-carboxylate (0.71 g, 2.55 mmol, 1.00 equiv), 1-iodo-4-(trifluoromethyl)benzene (0.83 g, 3.06 mmol, 1.20 equiv), Cs₂CO₃ (1.66 g, 5.10 mmol, 2.00 equiv) and XantPhos (0.03 g, 0.05 mmol, 0.02 equiv) in toluene (30.00 mL) was added Pd₂(dba)₃ (0.02 g, 0.025 mmol, 0.01 equiv). The reaction mixture was stirred for 12 h at 90° C. under nitrogen atmosphere. Desired product was detected by LCMS. The mixture was quenched with H₂O (50.00 mL) and then extracted with EA (3×50.00 mL). The combined organic layers were washed with brine (2×80.00 mL), dried over anhydrous Na₂SO₄. After filtration, the filtrate was concentrated under reduced pressure and the residue was purified by silica gel column chromatography, eluted with PE:EA (5:1) to afford tert-butyl 4-(2-{[4-(trifluoromethyl)phenyl]amino}pyridin-3-yl)piperazine-1-carboxylate (0.60 g, 55.68%) as a yellow solid. LCMS (Negative, ES, m/z): 421.2

### Step 3: Synthesis of 3-(piperazin-1-yl)-N-(4-(trifluoromethyl)phenyl)pyridin-2-amine

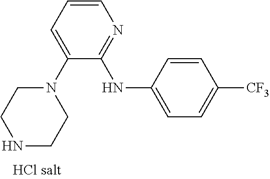 [SMILES: FC(F)(F)c1ccc(Nc2ncccc2N2CCNCC2)cc1]

To a stirred mixture of 2-[4-(2-oxo-1H-pyridine-3-amido)piperidin-1-yl]ethyl 7-[(3R)-3-[(tert-butoxycarbonyl)amino]-4-(2,4,5-trifluorophenyl)butanoyl]-3-(trifluoromethyl)-5H,6H,8H-imidazo[1,5-a]pyrazine-1-carboxylate (0.45 g, 0.56 mmol, 1.00 equiv) in DCM (1.00 mL) were added HCl(gas) in 1,4-dioxane (1.41 mL, 5.64 mmol, 10.00 equiv) at 0° C. The resulting mixture was stirred for 2 h at room temperature. The reaction was monitored by LCMS. The resulting mixture was concentrated under reduced pressure to afford 2-[4-(2-oxo-1H-pyridine-3-amido)piperidin-1-yl]ethyl 7-[(3R)-3-amino-4-(2,4,5-trifluorophenyl)butanoyl]-3-(trifluoromethyl)-5H,6H,8H-imidazo[1,5-a]pyrazine-1-carboxylate (0.35 g, 88.94%) as a yellow solid. LCMS:(Negative, ES, m/z): 321.1.

### Step 4: Synthesis of 1-[4-(2-{[4-(trifluoromethyl)phenyl]amino}pyridin-3-yl)piperazin-1-yl]prop-2-en-1-one

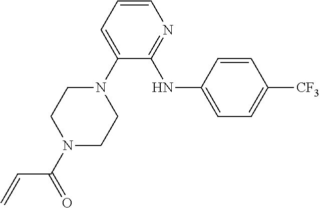 [SMILES: C=CC(=O)N1CCN(c2cccnc2Nc2ccc(C(F)(F)F)cc2)CC1]

Into a 10 ml bottle were added 3-(piperazin-1-yl)-N-[4-(trifluoromethyl)phenyl]pyridin-2-amine (0.30 g, 0.93 mmol, 1.00 equiv) and TEA (0.28 g, 2.79 mmol, 3.00 equiv) in DCM (1.00 mL) at 0° C. To the above mixture was added acryloyl chloride (0.09 g, 1.02 mmol, 1.10 equiv). The resulting mixture was stirred for additional 2 h at room temperature. The reaction was monitored by LCMS. The resulting mixture was quenched with 50.00 mL H₂O and extracted with ethyl acetate (50 mL×3). The combined organic layers were washed with brine (100.00 mL), dried over anhydrous Na₂SO₄. After filtration, the filtrate was concentrated under reduced pressure. The crude product was purified by (Column: Welch Xtimate C18 ExRS, 250 mm, 10 μm; Mobile Phase A: Water (0.05% NH₃·H₂O), Mobile Phase B: ACN; Flow rate: 90 mL/min; Gradient: 90% B to 90% B in 10 min, 90% B; Wave Length: 254 nm; RT1(min): 7; Number Of Runs: 5) to afford 1-[4-(2-{[4-(trifluoromethyl)phenyl]amino}pyridin-3-yl)piperazin-1-yl]prop-2-en-1-one (70.00 mg, 19.98%) as white solid. LCMS (Positive, ES, m/z): 377.2.¹H NMR (400 MHz, DMSO-d₆): δ 8.43 (s, 1H), 8.03-7.99 (m, 3H), 7.62 (d, J=8.8 Hz, 2H), 7.50 (d, J=7.6 Hz, 1H), 6.92-6.83 (m, 2H), 6.19-6.15 (m, 1H), 5.74-5.71 (m, 1H), 3.85-3.71 (m, 4H), 2.87-2.84 (m, 4H).

### Example 144: Synthesis of 2-fluoro-1-(4-(2-((6-(trifluoromethyl)pyridin-3-yl)amino)pyridin-3-yl)piperazin-1-yl)prop-2-en-1-one

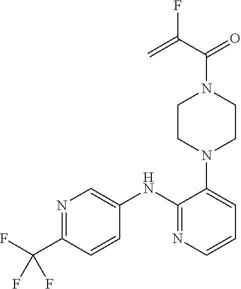 [SMILES: C=C(F)C(=O)N1CCN(c2cccnc2Nc2ccc(C(F)(F)F)nc2)CC1]

Following the general procedure in scheme 10, 3-(piperazin-1-yl)-N-(6-(trifluoromethyl)pyridin-3-yl)pyridin-2-amine was used. Offered 2-fluoro-1-(4-(2-((6-(trifluoromethyl)pyridin-3-yl)amino)pyridin-3-yl)piperazin-1-yl)prop-2-en-1-one as white solid. (Positive, ES, m/z):396.42. ¹H NMR (400 MHz, DMSO) δ 9.07 (d, J=8.1 Hz, 1H), 8.69-8.45 (m, 2H), 8.02 (d, J=8.5 Hz, 1H), 7.80 (s, 1H), 7.55 (s, 1H), 6.96 (d, J=8.6 Hz, 1H), 5.40-5.12 (m, 2H), 3.80 (s, 4H), 2.91 (d, J=9.4 Hz, 4H).

### Example 145: Synthesis of 1-(2-((6-(trifluoromethyl)pyridin-3-yl)amino)-3′,6′-dihydro-[3,4′-bipyridin]-1′(2′H)-yl)prop-2-en-1-one

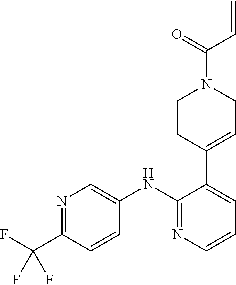 [SMILES: C=CC(=O)N1CC=C(c2cccnc2Nc2ccc(C(F)(F)F)nc2)CC1]

Following the general procedure in scheme 6, 3-chloro-N-(6-(trifluoromethyl)pyridin-3-yl)pyridin-2-amine was used in step 1. Offered 1-(2-((6-(trifluoromethyl)pyridin-3-yl)amino)-3′,6′-dihydro-[3,4′-bipyridin]-1′(2′H)-yl)prop-2-en-1-one as white solid. (Positive, ES, m/z):375.1. ¹H NMR (400 MHz, DMSO) δ 8.91 (d, J=2.5 Hz, 1H), 8.55 (d, J=7.8 Hz, 1H), 8.28 (d, J=8.9 Hz, 1H), 8.19 (dd, J=4.9, 1.8 Hz, 1H), 7.76 (d, J=8.7 Hz, 1H), 7.57 (d, J=7.5 Hz, 1H), 7.01 (dd, J=7.5, 4.9 Hz, 1H), 6.94-6.77 (m, 1H), 6.24-6.11 (m, 1H), 5.95 (d, 0.1=10.5 Hz, 1H), 5.82-5.69 (m, 1H), 4.38-4.10 (m, 2H), 3.80 (d, 0.1=6.1 Hz, 2H), 2.46-2.33 (m, 2H).

### Example 146: Synthesis of 2-fluoro-1-(2-((6-(trifluoromethyl)pyridin-3-yl)amino)-3′,6′-dihydro-[3,4′-bipyridin]-1′(2′H)-yl)prop-2-en-1-one

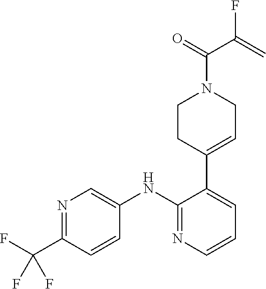 [SMILES: C=C(F)C(=O)N1CC=C(c2cccnc2Nc2ccc(C(F)(F)F)nc2)CC1]

Following the general procedure in scheme 10, N-(6-(trifluoromethyl)pyridin-3-yl)-1′,2′,3′,6′-tetrahydro-[3,4′-bipyridin]-2-amine was used. Offered 2-fluoro-1-(2-((6-(trifluoromethyl)pyridin-3-yl)amino)-3′,6′-dihydro-[3,4′-bipyridin]-1′(2′H)-yl)prop-2-en-1-one as white solid. (Positive, ES, m/z):393.1. ¹H NMR (400 MHz, DMSO) δ 8.91 (d, J=2.6 Hz, 1H), 8.56 (s, 1H), 8.32-8.13 (m, 2H), 7.76 (d, J=8.7 Hz, 1H), 7.58 (dd, J=7.5, 1.9 Hz, 1H), 7.01 (dd, J=7.5, 4.9 Hz, 1H), 5.95 (s, 1H), 5.43-5.12 (m, 2H), 4.34-4.13 (m, 2H), 3.78 (s, 2H), 3.30-3.17 (m, 2H).

### Example 147: Synthesis of 1-(2-((6-(trifluoromethoxy)pyridin-3-yl)amino)-3′,6′-dihydro-[3,4′-bipyridin]-1′(2′H)-yl)prop-2-en-1-one

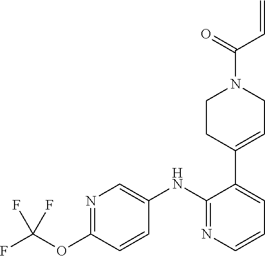 [SMILES: C=CC(=O)N1CC=C(c2cccnc2Nc2ccc(OC(F)(F)F)nc2)CC1]

Following the general procedure in scheme 6, 3-chloro-N-(6-(trifluoromethoxy)pyridin-3-yl)pyridin-2-amine was used in step 1. Offered 1-(2-((6-(trifluoromethoxy)pyridin-3-yl)amino)-3′,6′-dihydro-[3,4′-bipyridin]-1′(2′H)-yl)prop-2-en-1-one as white solid. (Positive, ES, m/z):391.2. ¹H NMR (400 MHz, DMSO) δ 8.55 (d, J=2.8 Hz, 1H), 8.21 (dd, J=9.3, 3.7 Hz, 2H), 8.10 (dd, J=4.8, 1.9 Hz, 1H), 7.57-7.43 (m, 1H), 7.23 (d, J=8.8 Hz, 1H), 7.00-6.75 (m, 2H), 6.24-6.08 (m, 1H), 5.93 (d, J=9.4 Hz, 1H), 5.81-5.67 (m, 1H), 4.44-4.12 (m, 2H), 3.81 (d, J=6.0 Hz, 2H), 2.47-2.30 (m, 2H).

### Example 148: Synthesis of 2-fluoro-1-(2-((6-(trifluoromethoxy)pyridin-3-yl)amino)-3′,6′-dihydro-[3,4′-bipyridin]-1′(2′H)-yl)prop-2-en-1-one

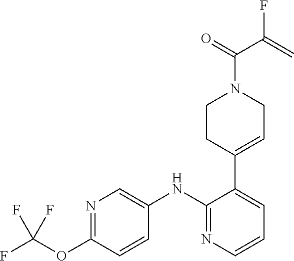 [SMILES: C=C(F)C(=O)N1CC=C(c2cccnc2Nc2ccc(OC(F)(F)F)nc2)CC1]

Following the general procedure in scheme 10, N-(6-(trifluoromethoxy)pyridin-3-yl)-1′,2′,3′,6′-tetrahydro-[3,4′-bipyridin]-2-amine was used. Offered 2-fluoro-1-(2-((6-(trifluoromethoxy)pyridin-3-yl)amino)-3′,6′-dihydro-[3,4′-bipyridin]-1′(2′H)-yl)prop-2-en-1-one as white solid. (Positive, ES, m/z):409.1. ¹H NMR (400 MHz, DMSO) δ 8.52 (d, J=2.8 Hz, 1H), 8.26-8.16 (m, 2H), 8.09 (dd, J=4.9, 1.8 Hz, 1H), 7.50 (dd, J=7.4, 1.9 Hz, 1H), 7.22 (d, J=8.8 Hz, 1H), 6.91 (dd, J=7.4, 4.9 Hz, 1H), 5.91 (s, 1H), 5.41-5.13 (m, 2H), 4.31-4.08 (m, 2H), 3.76 (d, I=5.7 Hz, 2H), 3.41 (d, I=1.0 Hz, 2H), 2.47 (s, 2H).

### Example 149: Synthesis of 1-(4-(2-((6-(trifluoromethyl)pyridin-3-yl)amino)pyridin-3-yl)piperidin-1-yl)prop-2-en-1-one

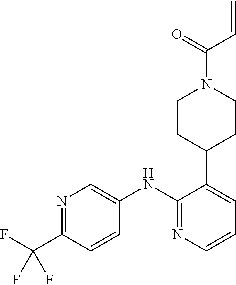 [SMILES: C=CC(=O)N1CCC(c2cccnc2Nc2ccc(C(F)(F)F)nc2)CC1]

Following the general procedure in scheme 7, tert-butyl 2-((6-(trifluoromethyl)pyridin-3-yl)amino)-3′,6′-dihydro-[3,4′-bipyridine]-1′(2′H)-carboxylate was used in step 1. Offered 1-(4-(2-((6-(trifluoromethyl)pyridin-3-yl)amino)pyridin-3-yl)piperidin-1-yl)prop-2-en-1-one as white solid. (Positive, ES, m/z):377.44. ¹H NMR (400 MHz, DMSO) δ 8.91 (d, J=2.5 Hz, 1H), 8.72 (s, 1H), 8.26 (dd, J=8.9, 2.5 Hz, 1H), 8.11 (dd, J=4.8, 1.7 Hz, 1H), 7.77 (d, J=8.7 Hz, 1H), 7.65 (dd, J=7.7, 1.8 Hz, 1H), 6.98 (dd, J=7.6, 4.8 Hz, 1H), 6.87 (dd, J=16.7, 10.4 Hz, 1H), 6.13 (dd, J=16.7, 2.5 Hz, 1H), 5.70 (dd, J=10.4, 2.5 Hz, 1H), 4.64 (d, J=13.0 Hz, 1H), 4.23 (d, J=13.6 Hz, 1H), 3.25 (q, J=12.7 Hz, 2H), 2.79 (t, J=12.7 Hz, 1H), 1.87 (d, J=12.9 Hz, 2H), 1.64-1.40 (m, 2H).

### Example 150: Synthesis of 2-fluoro-1-(4-(2-((6-(trifluoromethyl)pyridin-3-yl)amino)pyridin-3-yl)piperidin-1-yl)prop-2-en-1-one

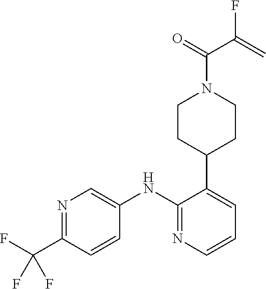 [SMILES: C=C(F)C(=O)N1CCC(c2cccnc2Nc2ccc(C(F)(F)F)nc2)CC1]

Following the general procedure in scheme 10, N3-(piperidin-4-yl)-N-(6-(trifluoromethyl)pyridin-3-yl)pyridin-2-amine was used. Offered 2-fluoro-1-(4-(2-((6-(trifluoromethyl)pyridin-3-yl)amino)pyridin-3-yl)piperidin-1-yl)prop-2-en-1-one as white solid. (Positive, ES, m/z):395.44. ¹H NMR (400 MHz, DMSO) δ 8.90 (d, J=2.5 Hz, 1H), 8.73 (s, 1H), 8.25 (dd, J=8.5, 2.5 Hz, 1H), 8.12 (dd, J=4.8, 1.8 Hz, 1H), 7.77 (d, J=8.7 Hz, 1H), 7.68 (dd, J=7.7, 1.8 Hz, 1H), 6.99 (dd, J=7.6, 4.8 Hz, 1H), 5.35-5.12 (m, 2H), 4.48 (s, 1H), 4.07 (s, 1H), 3.28 (d, J=12.0 Hz, 2H), 3.01-2.82 (m, 1H), 1.90 (d, J=13.0 Hz, 2H), 1.58 (s, 2H).

### Example 151: Synthesis of 1-(4-(2-((6-(trifluoromethoxy)pyridin-3-yl)amino)pyridin-3-yl)piperidin-1-yl)prop-2-en-1-one

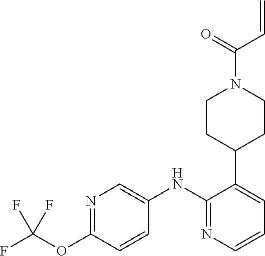 [SMILES: C=CC(=O)N1CCC(c2cccnc2Nc2ccc(OC(F)(F)F)nc2)CC1]

Following the general procedure in scheme 7, tert-butyl 2-((6-(trifluoromethoxy)pyri din-3-yl)amino)-3′,6′-dihydro-[3,4′-bipyridine]-1′(2′H)-carboxylate was used in step 1. Offered 1-(4-(2-((6-(trifluoromethoxy)pyridin-3-yl)amino)pyridin-3-yl)piperidin-1-yl)prop-2-en-1-one as white solid. (Positive, ES, m/z):393.1. ¹H NMR (400 MHz, DMSO) δ 8.59-8.35 (m, 2H), 8.19 (s, 1H), 8.02 (d, J=9.5 Hz, 1H), 7.57 (d, J=6.9 Hz, 1H), 7.24 (s, 1H), 6.96-6.78 (m, 2H), 6.12 (s, 1H), 5.69 (s, 1H), 4.63 (s, 1H), 4.22 (s, 1H), 3.21 (s, 2H), 2.79 (d, J=12.3 Hz, 1H), 1.87 (s, 2H), 1.49 (s, 2H).

### Example 152: Synthesis of 2-fluoro-1-(4-(2-((6-(trifluoromethoxy)pyridin-3-yl)amino)pyridin-3-yl)piperidin-1-yl)prop-2-en-1-one

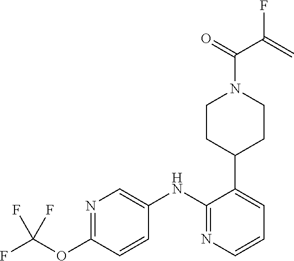 [SMILES: C=C(F)C(=O)N1CCC(c2cccnc2Nc2ccc(OC(F)(F)F)nc2)CC1]

Following the general procedure in scheme 10, N3-(piperidin-4-yl)-N-(6-(trifluoromethoxy)pyridin-3-yl)pyridin-2-amine was used. Offered 2-fluoro-1-(4-(2-((6-(trifluoromethoxy)pyridin-3-yl)amino)pyridin-3-yl)piperidin-1-yl)prop-2-en-1-one as white solid. (Positive, ES, m/z):411.1. ¹H NMR (400 MHz, DMSO) δ 8.54 (d, J=2.8 Hz, IH), 8.43 (s, 1H), 8.19 (dd, J=8.9, 2.8 Hz, 1H), 8.03 (dd, J=4.8, 1.7 Hz, 1H), 7.60 (dd, J=7.5, 1.8 Hz, 1H), 7.25 (d, J=8.8 Hz, 1H), 6.89 (dd, J=7.6, 4.8 Hz, 1H), 5.32-5.11 (m, 2H), 4.47 (s, 1H), 4.06 (s, 1H), 3.22 (d, J=11.9 Hz, 1H), 2.91 (s, 1H), 2.72-2.57 (m, 1H), 1.91 (d, J=12.9 Hz, 2H), 1.57 (s, 2H).

### Example 153: Synthesis of 1-(2-((6-(trifluoromethyl)pyridin-3-yl)oxy)-3′,6′-dihydro-[3,4′-bipyridin]-1′(2′H)-yl)prop-2-en-1-one

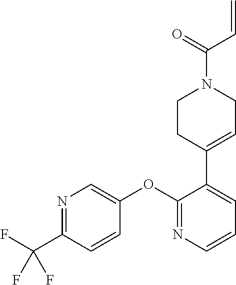 [SMILES: C=CC(=O)N1CC=C(c2cccnc2Oc2ccc(C(F)(F)F)nc2)CC1]

Following the general procedure in scheme 6, 3-chloro-2-((6-(trifluoromethyl)pyridin-3-yl)oxy)pyridine was used in step 1. Offered 1-(2-((6-(trifluoromethyl)pyridin-3-yl)oxy)-3′,6′-dihydro-[3,4′-bipyridin]-1′(2′H)-yl)prop-2-en-1-one as white solid. (Positive, ES, m/z):376.2. ¹H NMR (400 MHz, DMSO) δ 8.67 (d, J=2.6 Hz, 1H), 8.08 (dd, J=4.8, 1.8 Hz, 1H), 7.99 (d, J=8.6 Hz, 1H), 7.89 (dd, J=15.5, 8.0 Hz, 2H), 7.27 (dd, J=7.5, 4.8 Hz, 1H), 6.95-6.76 (m, 1H), 6.21-6.07 (m, 2H), 5.71 (d, J=8.1 Hz, 1H), 4.35-4.15 (m, 2H), 3.75 (dt, J=11.5, 5.9 Hz, 2H), 2.62 (s, 2H).

### Example 154: Synthesis of 2-fluoro-1-(2-((6-(trifluoromethyl)pyridin-3-yl)oxy)-3′,6′-dihydro-[3,4′-bipyridin]-1′(2′H)-yl)prop-2-en-1-one

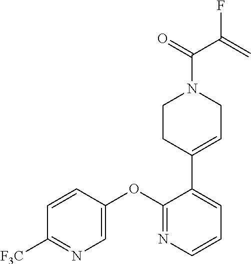 [SMILES: C=C(F)C(=O)N1CC=C(c2cccnc2Oc2ccc(C(F)(F)F)nc2)CC1]

Following the general procedure in scheme 10, 2-((6-(trifluoromethyl)pyridin-3-yl)oxy)-1′,2′,3′,6′-tetrahydro-3,4′-bipyridine was used. Offered 2-fluoro-1-(2-((6-(trifluoromethyl)pyridin-3-yl)oxy)-3′,6′-dihydro-[3,4′-bipyridin]-1′(2′H)-yl)prop-2-en-1-one as white solid. (Positive, ES, m/z):394.2. ¹H NMR (400 MHz, DMSO) δ 8.67 (d, J=2.6 Hz, 1H), 8.09 (dd, J=4.8, 1.8 Hz, 1H), 8.00 (d, J=8.6 Hz, 1H), 7.89 (ddd, J=14.4, 8.1, 2.3 Hz, 2H), 7.27 (dd, J=7.5, 4.8 Hz, 1H), 6.18 (s, 1H), 5.33 (d, J=16.9 Hz, 2H), 4.34-4.13 (m, 2H), 3.73 (t, 0.1=5.6 Hz, 2H), 2.65 (s, 2H).

### Example 155: Synthesis of 1-(4-(2-((6-(trifluoromethyl)pyridin-3-yl)oxy)pyridin-3-yl)piperidin-1-yl)prop-2-en-1-one

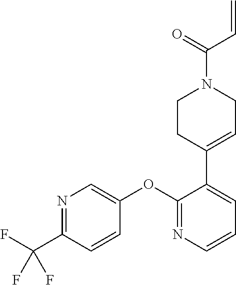 [SMILES: C=CC(=O)N1CC=C(c2cccnc2Oc2ccc(C(F)(F)F)nc2)CC1]

Following the general procedure in scheme 7, tert-butyl 2-((6-(trifluoromethyl)pyridin-3-yl)oxy)-3′,6′-dihydro-[3,4′-bipyridine]-1′(2′H)-carboxylate was used in step 1. Offered 1-(4-(2-((6-(trifluoromethyl)pyridin-3-yl)oxy)pyridin-3-yl)piperidin-1-yl)prop-2-en-1-one as white solid. (Positive, ES, m/z):378.1. ¹H NMR (400 MHz, DMSO) δ 8.67 (d, J=2.6 Hz, 1H), 8.01 (dd, J=6.5, 4.2 Hz, 2H), 7.93-7.79 (m, 2H), 7.21 (dd, J=7.5, 4.8 Hz, 1H), 6.86 (dd, J=16.7, 10.5 Hz, 1H), 6.12 (dd, J=16.7, 2.5 Hz, 1H), 5.69 (dd, J=10.4, 2.4 Hz, 1H), 4.63 (d, J=13.1 Hz, 1H), 4.22 (d, J=13.7 Hz, 1H), 3.24 (d, J=15.0 Hz, 2H), 2.77 (t, J=12.8 Hz, 1H), 1.95 (d, J=12.9 Hz, 2H), 1.63 (t, J=14.4 Hz, 2H).

### Example 156: Synthesis of 2-fluoro-1-(4-(2-((6-(trifluoromethyl)pyridin-3-yl)oxy)pyridin-3-yl)piperidin-1-yl)prop-2-en-1-one

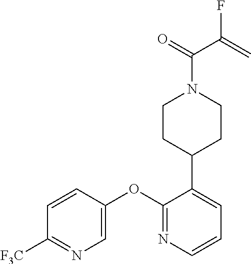 [SMILES: C=C(F)C(=O)N1CCC(c2cccnc2Oc2ccc(C(F)(F)F)nc2)CC1]

Following the general procedure in scheme 10, 3-(piperidin-4-yl)-2-((6-(trifluoromethyl)pyridin-3-yl)oxy)pyridine was used. Offered 2-fluoro-1-(4-(2-((6-(trifluoromethyl)pyridin-3-yl)oxy)pyridin-3-yl)piperidin-1-yl)prop-2-en-1-one as white solid. (Positive, ES, m/z):396.2. ¹H NMR (400 MHz, DMSO) δ 8.67 (d, J=2.6 Hz, 1H), 8.08-7.97 (m, 2H), 7.89 (ddd, J=9.6, 8.0, 2.3 Hz, 2H), 7.22 (dd, J=7.5, 4.9 Hz, 1H), 5.33-5.10 (m, 2H), 4.46 (s, 1H), 4.05 (s, 1H), 3.32-3.22 (m, 2H), 2.91 (s, 1H), 1.99 (d, J=13.0 Hz, 2H), 1.69 (s, 2H).

### Example 157: Synthesis of 1-(2-((2-(trifluoromethyl)pyrimidin-5-yl)oxy)-3′,6′-dihydro-[3,4′-bipyridin]-1′(2′H)-yl)prop-2-en-1-one

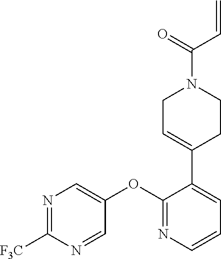 [SMILES: C=CC(=O)N1CC=C(c2cccnc2Oc2cnc(C(F)(F)F)nc2)CC1]

Following the general procedure in scheme 6, 5-((3-chloropyridin-2-yl)oxy)-2-(trifluoromethyl)pyrimidine was used in step 1. Offered 1-(2-((2-(trifluoromethyl)pyrimidin-5-yl)oxy)-3′,6′-dihydro-[3,4′-bipyridin]-1′(2′H)-yl)prop-2-en-1-one as white solid. (Positive, ES, m/z):377.36. ¹H NMR (400 MHz, DMSO) δ 9.09 (s, 2H), 8.09 (dd, J=4.9, 1.8 Hz, 1H), 7.90 (d, J=7.4 Hz, 1H), 7.30 (dd, J=7.5, 4.8 Hz, 1H), 6.95-6.74 (m, 1H), 6.26-6.11 (m, 2H), 5.72 (d, J=10.2 Hz, 1H), 4.39-4.14 (m, 2H), 3.77 (dt, J=11.1, 5.7 Hz, 2H), 2.66 (d, J=15.4 Hz, 2H).

### Example 158: Synthesis of 2-fluoro-1-(2-((2-(trifluoromethyl)pyrimidin-5-yl)oxy)-3′,6′-dihydro-[3,4′-bipyridin]-1′(2′H)-yl)prop-2-en-1-one

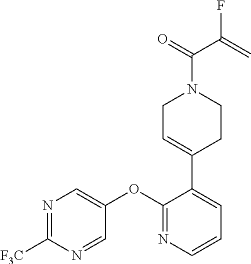 [SMILES: C=C(F)C(=O)N1CC=C(c2cccnc2Oc2cnc(C(F)(F)F)nc2)CC1]

Following the general procedure in scheme 10, 2-((2-(trifluoromethyl)pyrimidin-5-yl)oxy)-1′,2′,3′,6′-tetrahydro-3,4′-bipyridine was used. Offered 2-fluoro-1-(2-((2-(trifluoromethyl)pyrimidin-5-yl)oxy)-3′,6′-dihydro-[3,4′-bipyridin]-1′(2′H)-yl)prop-2-en-1-one as white solid. (Positive, ES, m/z):395.36. ¹H NMR (400 MHz, DMSO) δ 9.09 (s, 2H), 8.10 (dd, J=4.9, 1.8 Hz, 1H), 7.91 (dd, J=7.5, 1.9 Hz, 1H), 7.31 (dd, J=7.5, 4.9 Hz, 1H), 6.22 (s, 1H), 5.42-5.14 (m, 2H), 4.37-4.11 (m, 2H), 3.75 (t, J=5.7 Hz, 2H), 2.68 (s, 2H).

### Example 159: Synthesis of 1-(4-(2-((2-(trifluoromethyl)pyrimidin-5-yl)oxy)pyridin-3-yl)piperidin-1-yl)prop-2-en-1-one

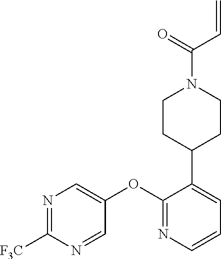 [SMILES: C=CC(=O)N1CCC(c2cccnc2Oc2cnc(C(F)(F)F)nc2)CC1]

Following the general procedure in scheme 7, tert-butyl 2-((2-(trifluoromethyl)pyrimidin-5-yl)oxy)-3′,6′-dihydro-[3,4′-bipyridine]-1′(2′H)-carboxylate was used in step 1. Offered 1-(4-(2-((2-(trifluoromethyl)pyrimidin-5-yl)oxy)pyridin-3-yl)piperidin-1-yl)prop-2-en-1-one as white solid. (Positive, ES, m/z):379.39. ¹H NMR (400 MHz, DMSO) δ 9.09 (s, 2H), 8.03 (dd, J=4.8, 1.8 Hz, 1H), 7.89 (dd, J=7.5, 1.8 Hz, 1H), 7.25 (dd, J=7.5, 4.8 Hz, 1H), 6.87 (dd, J=16.7, 10.4 Hz, 1H), 6.13 (dd, J=16.7, 2.5 Hz, 1H), 5.70 (dd, J=10.5, 2.5 Hz, 1H), 4.64 (d, J=13.0 Hz, 1H), 4.23 (d, J=13.7 Hz, 1H), 3.33-3.15 (m, 2H), 2.77 (t, J=12.7 Hz, 1H), 1.97 (d, 0.1=12.7 Hz, 2H), 1.71-1.48 (m, 2H).

### Example 160: Synthesis of2-fluoro-1-(4-(2-((2-(trifluoromethyl)pyrimidin-5-yl)oxy)pyridin-3-yl)piperidin-1-yl)prop-2-en-1-one

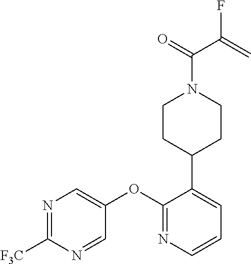 [SMILES: C=C(F)C(=O)N1CCC(c2cccnc2Oc2cnc(C(F)(F)F)nc2)CC1]

Following the general procedure in scheme 10, 5-((3-(piperidin-4-yl)pyridin-2-yl)oxy)-2-(trifluoromethyl)pyrimidine was used. Offered 2-fluoro-1-(4-(2-((2-(trifluoromethyl)pyrimidin-5-yl)oxy)pyridin-3-yl)piperidin-1-yl)prop-2-en-1-one as white solid. (Positive, ES, m/z):397.39. ¹H NMR (400 MHz, DMSO) δ 9.09 (s, 2H), 8.03 (dd, J=4.8, 1.8 Hz, 1H), 7.92 (d, J=7.4 Hz, 1H), 7.26 (dd, J=7.5, 4.8 Hz, 1H), 5.34-5.11 (m, 2H), 4.47 (s, 1H), 4.06 (s, 1H), 3.30 (d, J=14.6 Hz, 2H), 2.90 (s, 1H), 2.01 (d, J=13.0 Hz, 2H), 1.70 (s, 2H).

### Example 161: Synthesis of 1-(4-(3-(4,4-difluoropiperidine-1-carbonyl)pyridin-2-yl)piperazin-1-yl)prop-2-en-1-one

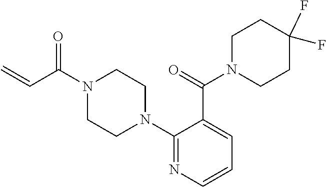 [SMILES: C=CC(=O)N1CCN(c2ncccc2C(=O)N2CCC(F)(F)CC2)CC1]

Following the general procedure in scheme 18, 2-chloronicotinic acid was used in step 1 and 4,4-difluoropiperidine was used in step 2. Offered 1-(4-(3-(4,4-difluoropiperidine-1-carbonyl)pyridin-2-yl)piperazin-1-yl)prop-2-en-1-one as white solid. (Positive, ES, m/z):365.32. H NMR (400 MHz, DMSO) δ 8.28 (dd, J=4.8, 1.9 Hz, 1H), 7.62 (dd, J=7.4, 1.9 Hz, 1H), 6.95 (dd, J=7.4, 4.8 Hz, 1H), 6.83 (dd, J=16.7, 10.5 Hz, 1H), 6.15 (dd, J=16.7, 2.4 Hz, 1H), 5.71 (dd, J=10.4, 2.4 Hz, 1H), 4.00-3.85 (m, 1H), 3.82-3.50 (m, 5H), 3.34-3.21 (m, 6H), 2.15-1.87 (m, 4H).

### Example 162: Synthesis of 1-(4-(2-((4-(trifluoromethyl)phenyl)amino)phenyl)piperazin-1-yl)prop-2-en-1-one

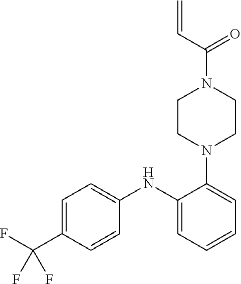 [SMILES: C=CC(=O)N1CCN(c2ccccc2Nc2ccc(C(F)(F)F)cc2)CC1]

Following the general procedure in scheme 3, 2-chloroaniline was used in step 1. Offered 1-(4-(2-((4-(trifluoromethyl)phenyl)amino)phenyl)piperazin-1-yl)prop-2-en-1-one as white solid. (Positive, ES, m/z): 398.28. ¹H NMR (400 MHz, DMSO) δ 7.94 (s, 1H), 7.50 (d, J=8.5 Hz, 2H), 7.33-7.21 (m, 1H), 7.16-6.95 (m, 5H), 6.82 (dd, J=16.6, 10.4 Hz, 1H), 6.13 (dd, J=16.7, 2.4 Hz, 1H), 5.69 (dd, J=10.4, 2.4 Hz, 1H), 3.60 (s, 4H), 2.92-2.76 (m, 4H).

### Example 164. Synthesis of 1-(4-(3-(4-(trifluoromethyl)benzoyl)-1H-pyrazol-1-yl)piperidin-1-yl)prop-2-en-1-one (Example 163) and 1-(4-(5-(4-(trifluoromethyl)benzoyl)-1H-pyrazol-1-yl)piperidin-1-yl)prop-2-en-1-one

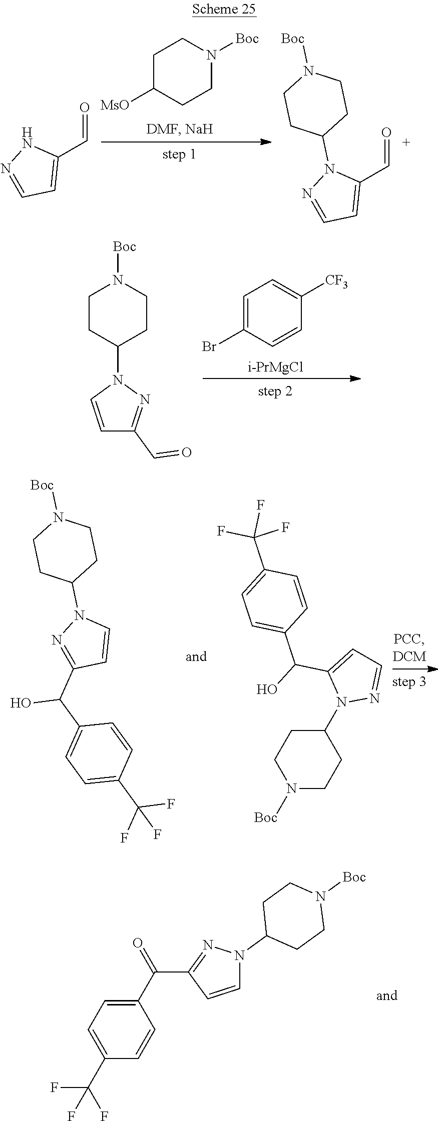

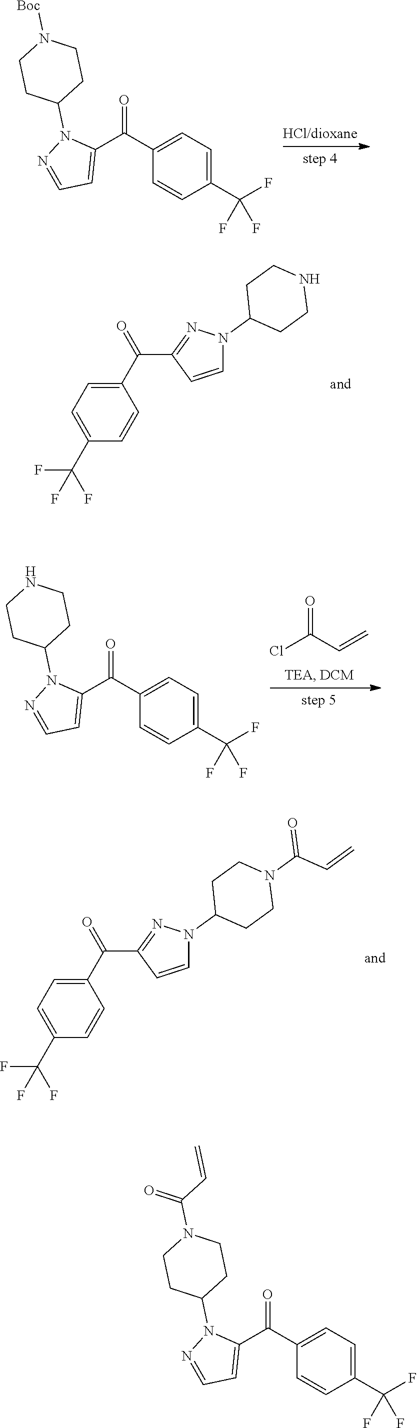

### Step 1: Synthesis of 4-(5-formyl-1IH-pyrazol-1-yl)piperidine-1-carboxylate and tert-butyl 4-(3-formyl-1H-pyrazol-1-yl)piperidine-1-carboxylate

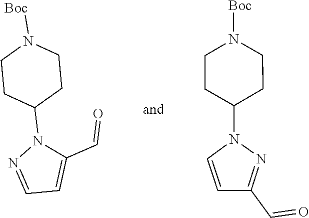 [SMILES: CC(C)(C)OC(=O)N1CCC(n2nccc2C=O)CC1 ; CC(C)(C)OC(=O)N1CCC(n2ccc(C=O)n2)CC1]

A solution/mixture of 2H-pyrazole-3-carbaldehyde (6 g, 62.44 mmol, 1 equiv) and tert-butyl 4-(methanesulfonyloxy) piperidine-1-carboxylate (29.65 g, 106.15 mmol, 1.7 equiv) and NaH (2.40 g, 99.91 mmol, 1.6 equiv) in DMF (50 mL) was stirred for 8 h at 80° C. under nitrogen atmosphere. The resulting mixture was diluted with water (50 mL). The resulting mixture was extracted with EtOAc (3×70 mL). The combined organic layers were washed with brine (3×30 mL), dried over anhydrous Na₂SO₄. After filtration, the filtrate was concentrated under reduced pressure. The residue was purified by silica gel column chromatography, eluted with PE/EA (1:1) to afford a mixture of tert-butyl 4-(5-formyl-1H-pyrazol-1-yl)piperidine-1-carboxylate and tert-butyl 4-(3-formyl-1H-pyrazol-1-yl)piperidine-1-carboxylate (9 g) as a yellow solid.

### Step 2: Synthesis of tert-butyl 4-(3-(hydroxy(4-(trifluoromethyl)phenyl)methyl)-1H-pyrazol-1-yl)piperidine-1-carboxylate and tert-butyl 4-(5-(hydroxy(4-(trifluoromethyl)phenyl)methyl)-1H-pyrazol-1-yl)piper dine-1-carboxylate

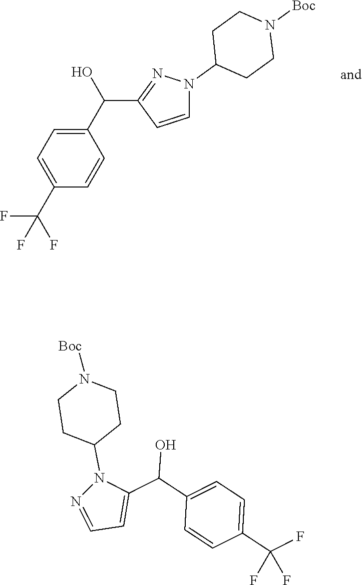 [SMILES: CC(C)(C)OC(=O)N1CCC(n2ccc(C(O)c3ccc(C(F)(F)F)cc3)n2)CC1 ; CC(C)(C)OC(=O)N1CCC(n2nccc2C(O)c2ccc(C(F)(F)F)cc2)CC1]

A solution of 1-bromo-4-(trifluoromethyl)benzene (18.16 g, 80.69 mmol, 5 equiv) and chlo ro(isopropyl)magnesium (50 mL) in THE (200 mL) was stirred for 40 min at 0° C. under nitrogen atmosphere. A mixture of 3&3A (9 g, 16.14 mmol, 1 equiv) was dropwised to the above Grignard reagent solution. The solution was stirred for 2 h at 0° C. under nitrogen atmosphere. The resulting mixture was diluted with water (200 mL). The resulting mixture was extracted with EtOAc (2×200 mL). The combined organic layers were washed with brine (2×100 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% NH₃·H₂O), 10% to 50% gradient in 20 min; detector, UV 254 nm to afford a mixture of tert-butyl 4-(3-(hydroxy(4-(trifluoromethyl)phenyl)methyl)-1H-pyrazol-1-yl)piperidine-1-carboxylate and tert-butyl 4-(5-(hydroxy(4-(trifluoromethyl)phenyl)methyl)-1H-pyrazol-1-yl)piperidine-1-carboxylate (4 g) as a yellow solid.

### Step 3: Synthesis of tert-butyl 4-(3-(4-(trifluoromethyl)benzoyl)-1H-pyrazol-1-yl)piperidine-1-carboxylate and tert-butyl 4-(5-(4-(trifluoromethyl)benzoyl)-1H-pyrazol-1-yl)piperidine-1-carboxylate

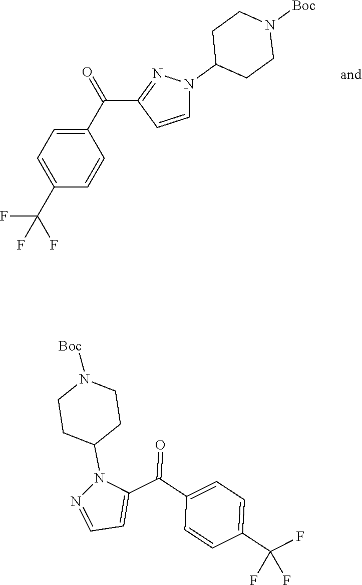 [SMILES: CC(C)(C)OC(=O)N1CCC(n2ccc(C(=O)c3ccc(C(F)(F)F)cc3)n2)CC1 ; CC(C)(C)OC(=O)N1CCC(n2nccc2C(=O)c2ccc(C(F)(F)F)cc2)CC1]

Into a 100 mL round-bottom flask were added a mixture of 4&4A (4 g, 9.40 mmol, 1 equiv), PCC (4.05 g, 18.80 mmol, 2 equiv) and DCM (40 mL, 629.22 mmol, 66.93 equiv) at room temperature. The resulting mixture was stirred for 3 h at room temperature. The resulting mixture was diluted with water (100 mL). The resulting mixture was extracted with CH₂Cl₂ (2×100 mL). The combined organic layers were washed with water (1×100 mL), dried over anhydrous Na₂SO₄. After filtration, the filtrate was concentrated under reduced pressure. The residue was purified by silica gel column chromatography, eluted with PE/EA (1:1) to afford crude product. The crude product was further purified by reverse phase flash with the following conditions: (column, C18 silica gel; mobile phase, MeCN in Water (0.1% NH₃·H₂O), 20% to 70% gradient in 20 min; detector, UV 254 nm) to afford tert-butyl 4-(3-(4-(trifluoromethyl)benzoyl)-1H-pyrazol-1-yl)piperidine-1-carboxylate (110 mg) and tert-butyl 4-(5-(4-(trifluoromethyl)benzoyl)-1H-pyrazol-1-yl)piperidine-1-carboxylate (230 mg).

### Step 4: Synthesis of (1-(piperidin-4-yl)-1H-pyrazol-3-yl)(4-(trifluoromethyl)phenyl)methanone

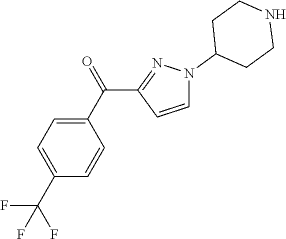 [SMILES: O=C(c1ccc(C(F)(F)F)cc1)c1ccn(C2CCNCC2)n1]

Into a 40 mL vial were added tert-butyl 4-(3-(4-(trifluoromethyl)benzoyl)-1H-pyrazol-1-yl) piperidine-1-carboxylate (110 mg, 0.26 mmol, 1 equiv), HCl(gas)in 1,4-dioxane (1 mL, 32.91 mmol, 126.70 equiv) and DCM (3 mL, 47.19 mmol, 181.66 equiv) at room temperature. The resulting mixture was stirred for 1 h at room temperature. The resulting mixture was concentrated under reduced pressure. The crude product was used in the next step directly without further purification.

### Step 5: Synthesis of 1-(4-(3-(4-(trifluoromethyl)benzoyl)-1H-pyrazol-1-yl)piperidin-1-yl)prop-2-en-1-one (Example 163)

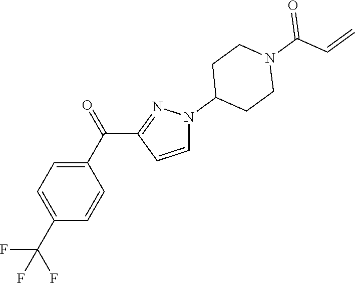 [SMILES: C=CC(=O)N1CCC(n2ccc(C(=O)c3ccc(C(F)(F)F)cc3)n2)CC1]

A solution (1-piperidin-4-yl) 1H-pyrazol-3-yl) (4-(trifluoromethyl)phenyl) methanone (100 mg, 0.31 mmol, 1 equiv) in DCM was treated with TEA (93.89 mg, 0.93 mmol, 3 equiv) for 5 min at 0° C. under nitrogen atmosphere followed by the addition of acryloyl chloride (27.99 mg, 0.31 mmol, 1 equiv) dropwise at 0° C. The resulting mixture was stirred for 1 h at room temperature. The resulting mixture was diluted with water (10 mL). The resulting mixture was extracted with CH₂Cl₂ (2×10 mL). The combined organic layers were washed with water (2×10 mL), dried over anhydrous Na₂SO₄. After filtration, the filtrate was concentrated under reduced pressure. The crude product was purified by reverse phase flash with the following conditions (column, C18 silica gel; mobile phase, MeCN in Water (0.1% NH₃·H₂O), 10% to 60% gradient in 20 min; detector, UV 254 nm) to afford 1-(4-(3-(4-(trifluoromethyl)benzoyl)-1H-pyrazol-1-yl) piperidin-1-yl) prop-2-en-1-one (63 mg, 53.98%) as a white oil. ¹H NMR (400 MHz, DMSO-d₆) δ 8.32 (d, J=8.1 Hz, 2H), 8.10 (d, J=2.5 Hz, 1H), 7.93 (d, 1=8.1 Hz, 2H), 6.98 (d, J=2.5 Hz, 1H), 6.87 (m, 1H), 6.13 (m, 1H), 5.70 (m, 1H), 4.71-4.62 (m, 1H), 4.55 (m, 1H), 4.20 (m, 1H), 3.24 (m, 1H), 2.86 (m, 1H), 2.14 (m, 2H), 1.96-1.81 (m, 2H).

### Step 4: Synthesis of (1-(piperidin-4-yl)-1H-pyrazol-5-yl)(4-(trifluoromethyl)phenyl)methanone

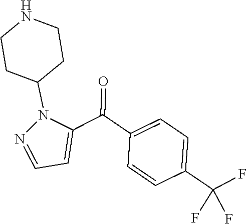 [SMILES: O=C(c1ccc(C(F)(F)F)cc1)c1ccnn1C1CCNCC1]

Into a 40 mL vial were added tert-butyl 4-(5-(4-(trifluoromethyl)benzoyl)-1H-pyrazol-1-yl) piperidine-1-carboxylate (230 mg, 0.54 mmol, 1 equiv), HCl(gas)in 1,4-dioxane (3 mL, 98.74 mmol, 181.78 equiv) and DCM (9 mL, 141.57 mmol, 260.64 equiv) at room temperature. The resulting mixture was stirred for 1 h at room temperature. The resulting mixture was concentrated under reduced pressure. The crude product was used in the next step directly without further purification.

### Step 5: Synthesis of 1-(4-(5-(4-(trifluoromethyl)benzoyl)-1H-pyrazol-1-yl)piperidin-1-yl)prop-2-en-1-one

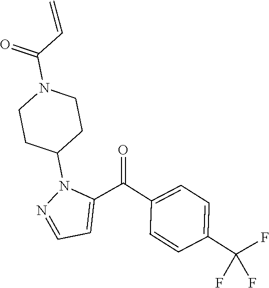 [SMILES: C=CC(=O)N1CCC(n2nccc2C(=O)c2ccc(C(F)(F)F)cc2)CC1]

A solution of (1-(piperidin-4-yl)-1H-pyrazol-3-yl) (4-(trifluoromethyl)phenyl) methanone (220 mg, 0.68 mmol, 1 equiv) in DCM (5 mL) was treated with TEA (206.57 mg, 2.04 mmol, 3 equiv) for 5 min at 0° C. followed by the addition of acryloyl chloride (61.59 mg, 0.68 mmol, 1 equiv) dropwise at 0° C. The resulting mixture was stirred for 1 h at room temperature. The resulting mixture was diluted with water (10 mL). The resulting mixture was extracted with CH₂Cl₂ (2×20 mL), dried over anhydrous Na₂SO₄. After filtration, the filtrate was concentrated under reduced pressure. The crude product was purified by reverse phase flash with the following conditions (column, C18 silica gel; mobile phase, MeCN in Water (0.1% NH₃·H₂O), 10% to 60% gradient in 20 min; detector, UV 254 nm) to afford 1-(4-(5-(4-(trifluoromethyl)benzoyl)-1H-pyrazol-1-yl)piperidin-1-yl)prop-2-en-1-one (60 mg, 23.32%) as a white solid. 1H NMR (400 MHz, DMSO-d₆) δ 8.05 (d, J=8.1 Hz, 2H), 7.96 (d, J=8.2 Hz, 2H), 7.68 (d, J=2.0 Hz, 1H), 6.87 (m, 1H), 6.79 (s, 1H), 6.14 (m, 1H), 5.71 (m, 1H), 5.23-5.13 (m, 1H), 4.57 (d, J=13.2 Hz, 1H), 4.22 (d, J=13.8 Hz, 1H), 3.23 (m, 1H), 2.83 (m, 1H), 2.15-2.04 (m, 2H), 2.00-1.86 (m, 2H).

### Example 165: Synthesis of 1-(4-(5-methyl-3-(4-(trifluoromethyl)benzoyl)-1H-pyrazol-1-yl)piperidin-1-yl)prop-2-en-1-one

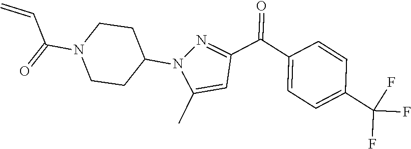 [SMILES: C=CC(=O)N1CCC(n2nc(C(=O)c3ccc(C(F)(F)F)cc3)cc2C)CC1]

Following the general procedure in scheme 25, 3-methyl-1H-pyrazole-5-carbaldehyde was used in step 1. Offered 1-(4-(5-methyl-3-(4-(trifluoromethyl)benzoyl)-1H-pyrazol-1-yl)piperidin-1-yl)prop-2-en-1-one as white solid. (Positive, ES, m/z): 392.34. ¹H NMR (400 MHz, DMSO) δ 8.30 (d, J=8.0 Hz, 2H), 7.92 (d, J=8.0 Hz, 2H), 6.91-6.80 (m, 1H), 6.75 (d,J=1.3 Hz, 1H), 6.13 (ddd, J=16.8, 2.5, 1.1 Hz, 1H), 5.70 (ddd, J=10.5, 2.5, 1.0 Hz, 1H), 4.60 (q, J=14.4 Hz, 2H), 4.20 (d, J=13.8 Hz, 1H), 3.26 (d, J=11.9 Hz, 1H), 2.87 (t, J=12.7 Hz, 1H), 2.02 (d, J=12.6 Hz, 2H), 1.87 (d, J=13.9 Hz, 2H).

### Example 166: Synthesis of 1-(4-(3-methyl-5-(4-(trifluoromethyl)benzoyl)-1H-pyrazol-1-yl)piperidin-1-yl)prop-2-en-1-one

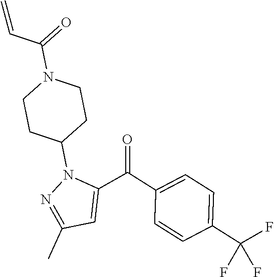 [SMILES: C=CC(=O)N1CCC(n2nc(C)cc2C(=O)c2ccc(C(F)(F)F)cc2)CC1]

Following the general procedure in scheme 25, 3-methyl-1H-pyrazole-5-carbaldehyde was used in step 1. Offered 1-(4-(3-methyl-5-(4-(trifluoromethyl)benzoyl)-1H-pyrazol-1-yl)piperidin-1-yl)prop-2-en-1-one as white solid. (Positive, ES, m/z): 392.38. ¹H NMR (400 MHz, DMSO) δ 8.04 (d, J=8.1 Hz, 2H), 7.96 (d, J=8.2 Hz, 2H), 6.88 (dd, J=16.7, 10.4 Hz, 1H), 6.57 (s, 1H), 6.14 (dd, J=16.7, 2.5 Hz, 1H), 5.71 (dd, J=10.4, 2.4 Hz, 1H), 5.12 (td, J=11.2, 5.5 Hz, 1H), 4.57 (d, J=13.2 Hz, 1H), 4.21 (d, J=13.9 Hz, 1H), 3.23 (t, J=12.8 Hz, 1H), 2.81 (t, J=12.9 Hz, 1H), 2.23 (s, 3H), 2.08-1.73 (m, 4H).

### Example 167: Synthesis of 4-(dimethylamino)-1-(4-(3-((4-(trifluoromethyl)phenyl)amino)pyrazin-2-yl)piperazin-1-yl)but-2-en-1-one

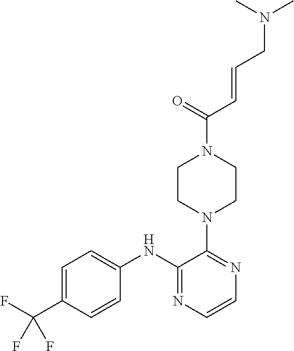 [SMILES: CN(C)C/C=C/C(=O)N1CCN(c2nccnc2Nc2ccc(C(F)(F)F)cc2)CC1]

Following the general procedure in scheme 10, (E)-4-(dimethylamino)but-2-enoic acid was used. Offered 1-(4-(2-((4-(trifluoromethyl)phenyl)amino)phenyl)piperazin-1-yl)prop-2-en-1-one as white solid. (Positive, ES, m/z): 435.32. ¹H NMR (400 MHz, DMSO) δ 8.63 (d, J=2.4 Hz, 1H), 8.02-7.89 (m, 3H), 7.85 (d, J=2.8 Hz, 1H), 7.65 (d, J=8.6 Hz, 2H), 6.82 (d, J=15.2 Hz, 1H), 6.65 (dt, J=15.0, 6.6 Hz, 1H), 3.79 (s, 4H), 3.52 (s, 6H), 3.15 (d, J=6.1 Hz, 6H).

### Example 168: Synthesis of 1-(4-(3-((4-(trifluoromethyl)phenyl)amino)pyrazin-2-yl)piperazin-1-yl)ethan-1-one

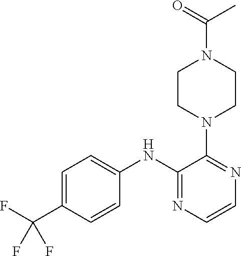 [SMILES: CC(=O)N1CCN(c2nccnc2Nc2ccc(C(F)(F)F)cc2)CC1]

Following the general procedure in scheme 1, acetyl chloride was used in step 4. Offered 1-(4-(3-((4-(trifluoromethyl)phenyl)amino)pyrazin-2-yl)piperazin-1-yl)ethan-1-one as white solid. (Positive, ES, m/z): 366.2. ¹H NMR (400 MHz, DMSO) δ 8.61 (s, 1H), 7.94-7.87 (m, 3H), 7.85 (d, J=2.8 Hz, 1H), 7.65 (d, J=8.6 Hz, 2H), 3.68 (q, J=4.9 Hz, 4H), 3.21-3.03 (m, 4H), 2.05 (s, 3H).

### Example 169: Synthesis of 2-chloro-2-fluoro-1-(4-(3-((4-(trifluoromethyl)phenyl)amino)pyrazin-2-yl)piperazin-1-yl)ethan-1-one

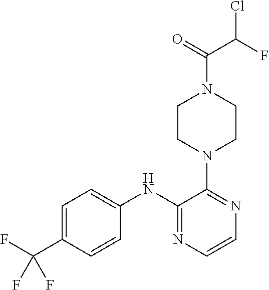 [SMILES: O=C(C(F)Cl)N1CCN(c2nccnc2Nc2ccc(C(F)(F)F)cc2)CC1]

Following the general procedure in scheme 1, 2-chloro-2-fluoroacetyl chloride was used in step 4. Offered 2-chloro-2-fluoro-1-(4-(3-((4-(trifluoromethyl)phenyl)amino)pyrazin-2-yl)piperazin-1-yl)ethan-1-one as white solid. (Positive, ES, m/z): 418.21. ¹H NMR (400 MHz, DMSO) δ 8.63 (s, 1H), 7.98-7.87 (m, 3H), 7.85 (d, J=2.8 Hz, 1H), 7.65 (d, J=8.6 Hz, 2H), 7.44-7.27 (m, 1H), 3.87-3.61 (m, 4H), 3.18 (dt, J=15.4, 4.3 Hz, 4H).

### Example 170: Synthesis of 2-chloro-1-(4-(3-((4-(trifluoromethyl)phenyl)amino)pyrazin-2-yl)piperazin-1-yl)ethan-1-one

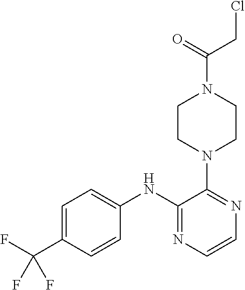 [SMILES: O=C(CCl)N1CCN(c2nccnc2Nc2ccc(C(F)(F)F)cc2)CC1]

Following the general procedure in scheme 1, 2-chloroacetyl chloride was used in step 4. Offered 2-chloro-1-(4-(3-((4-(trifluoromethyl)phenyl)amino)pyrazin-2-yl)piperazin-1-yl)ethan-1-one as white solid. (Positive, ES, m/z): 400.26. ¹H NMR (400 MHz, DMSO) δ 8.63 (s, 1H), 7.97-7.86 (m, 3H), 7.85 (d, J=2.8 Hz, 1H), 7.65 (d, J=8.6 Hz, 2H), 4.45 (s, 2H), 3.82-3.65 (m, 4H), 3.26-3.05 (m, 4H).

### Example 171: Synthesis of 3-oxo-3-(4-(3-((4-(trifluoromethyl)phenyl)amino)pyrazin-2-yl)piperazin-1-yl)propanenitrile

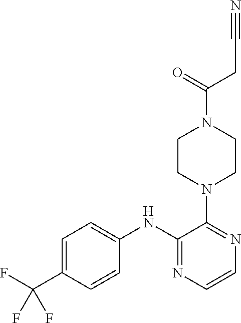 [SMILES: N#CCC(=O)N1CCN(c2nccnc2Nc2ccc(C(F)(F)F)cc2)CC1]

Following the general procedure in scheme 10, 2-cyanoacetic acid was used. Offered 3-oxo-3-(4-(3-((4-(trifluoromethyl)phenyl)amino)pyrazin-2-yl)piperazin-1-yl)propanenitrile as white solid. (Positive, ES, m/z): 391.27. ¹H NMR (400 MHz, DMSO) δ 8.63 (s, 1H), 7.93 (s, 1H), 7.91-7.88 (m, 2H), 7.84 (d, J=2.8 Hz, 1H), 7.65 (d, J=8.5 Hz, 2H), 4.12 (s, 2H), 3.71 (t, J=5.3 Hz, 2H), 3.59 (d, J=5.6 Hz, 2H), 3.23-3.07 (m, 4H).

### Example 172: Synthesis of oxiran-2-yl(4-(3-((4-(trifluoromethyl)phenyl)amino)pyrazin-2-yl)piperazin-1-yl)methanone

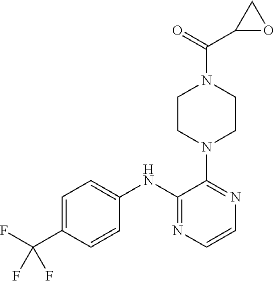 [SMILES: O=C(C1CO1)N1CCN(c2nccnc2Nc2ccc(C(F)(F)F)cc2)CC1]

Following the general procedure in scheme 10, oxirane-2-carboxylic acid was used. Offered oxiran-2-yl(4-(3-((4-(trifluoromethyl)phenyl)amino)pyrazin-2-yl)piperazin-1-yl)methanone as white solid. (Positive, ES, m/z): 394.28. ¹H NMR (400 MHz, DMSO) δ 8.64 (s, 1H), 7.98-7.88 (m, 3H), 7.85 (d, J=2.8 Hz, 1H), 7.65 (d, J=8.6 Hz, 2H), 3.97-3.80 (m, 3H), 3.71 (d, J=6.6 Hz, 2H), 3.26-3.08 (m, 4H), 2.93 (dd, J=6.3, 4.3 Hz, 1H), 2.81 (dd, J=6.3, 2.6 Hz, 1H).

### Example 173. Synthesis of 1-(4-(3-((4-(trifluoromethyl)phenyl)amino)pyrazin-2-yl)piperazin-1-yl)prop-2-yn-1-one

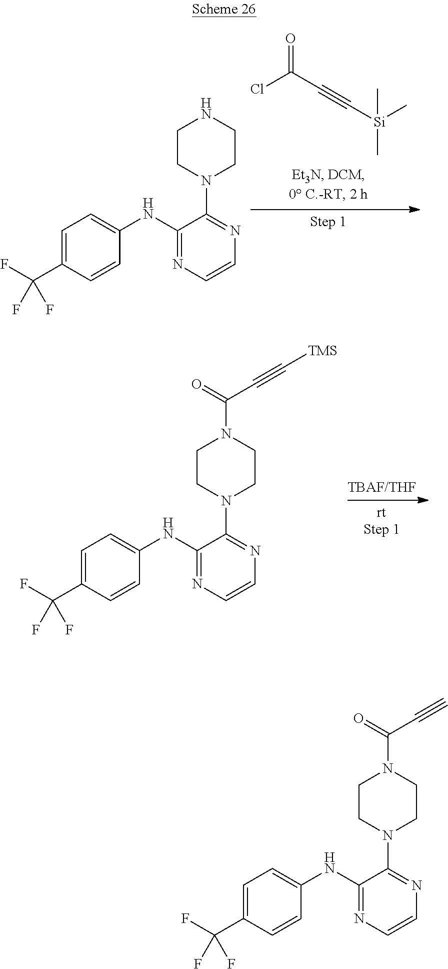

### Step 1: Synthesis of 1-(4-(3-((4-(trifluoromethyl)phenyl)amino)pyrazin-2-yl)piperazin-1-yl)-3-(trimethylsilyl) prop-2-yn-1-one

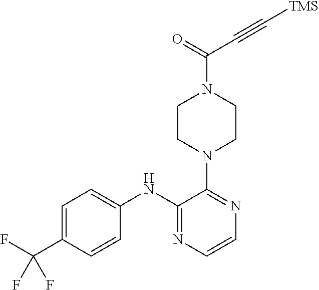 [SMILES: C[Si](C)(C)C#CC(=O)N1CCN(c2nccnc2Nc2ccc(C(F)(F)F)cc2)CC1]

3-(piperazin-1-yl)-N-(4-(trifluoromethyl)phenyl)pyrazin-2-amine (100 mg, 0.31 mmol, 1.00 equiv) and TEA (128 mmL, 0.77 mmol, 2.50 equiv) in DCM (2.00 mL) at 0° C. To the above mixture was added 3-(trimethylsilyl)propioloyl chloride (56 mmL, 0.37 mmol, 1.20 equiv). The resulting mixture was stirred for additional 0.5 h at 0° C. The reaction was monitored by LCMS. The resulting mixture was added 20.00 mL H₂O and extracted with ethyl acetate (20.00 mL×3). The combined organic layers were washed with brine (50.00 mL), dried over anhydrous Na₂SO₄. After filtration, the filtrate was concentrated under reduced pressure. The crude product was used for next step without further purification.

### Step 2: Synthesis of 1-(4-(3-((4-(trifluoromethyl)phenyl)amino)pyrazin-2-yl)piperazin-1-yl)prop-2-yn-1-one

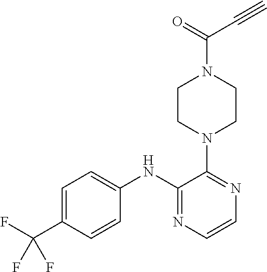 [SMILES: C#CC(=O)N1CCN(c2nccnc2Nc2ccc(C(F)(F)F)cc2)CC1]

To a stirred mixture of 1-(4-(3-((4-(trifluoromethyl)phenyl)amino)pyrazin-2-yl)piperazin-1-yl)-3-(trimethylsilyl) prop-2-yn-1-one (108 mg, 0.24 mmol, 1.00 equiv) in THE (2.00 mL) were added TBAF in THF (844 mmL, 0.84 mmol, 3.5 equiv) at room temperature. The resulting mixture was stirred for 2 h at room temperature. The resulting mixture was concentrated under reduced pressure, the crude product was purified by silica gel column chromatography, eluted with DCM:EA(4:1) to afford 1-(4-(3-((4-(trifluoromethyl)phenyl)amino)pyrazin-2-yl)piperazin-1-yl)prop-2-yn-1-one (61 mg, 67.34% yield) as white solid. (Positive, ES, m/z): 376.22. ¹H NMR (400 MHz, DMSO) δ 8.61 (s, 1H), 7.96-7.89 (m, 3H), 7.85 (d, J=2.8 Hz, 1H), 7.65 (d, J=8.5 Hz, 2H), 4.61 (s, 1H), 4.00-3.88 (m, 2H), 3.75 (dd, J=6.3, 4.0 Hz, 2H), 3.22 (t, J=5.2 Hz, 2H), 3.14 (t, J=5.2 Hz, 2H).

### Example 174: Synthesis of 1-(4-(3-((4-(trifluoromethyl)phenyl)amino)pyrazin-2-yl)piperazin-1-yl)but-2-yn-1-one

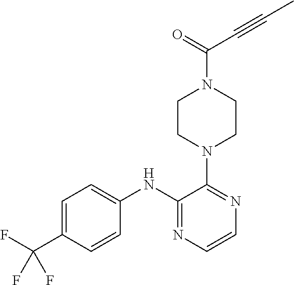 [SMILES: CC#CC(=O)N1CCN(c2nccnc2Nc2ccc(C(F)(F)F)cc2)CC1]

Following the general procedure in scheme 1, but-2-ynoyl chloride was used in step 4. Offered 1-(4-(3-((4-(trifluoromethyl)phenyl)amino)pyrazin-2-yl)piperazin-1-yl)but-2-yn-1-one as white solid. (Positive, ES, m/z): 390.28. ¹H NMR (400 MHz, DMSO) δ 8.61 (s, 1H), 7.99-7.88 (m, 3H), 7.85 (d, J=2.8 Hz, 1H), 7.65 (d, J=8.6 Hz, 2H), 3.92 (t, J=5.1 Hz, 2H), 3.73 (t, J=5.1 Hz, 2H), 3.27-3.08 (m, 4H), 2.04 (s, 3H).

### Example 175: Synthesis of 2-(4-(3-((4-(trifluoromethyl)phenyl)amino)pyrazin-2-yl)piperazin-1-yl)acetonitrile

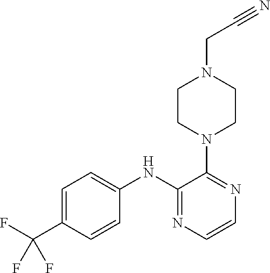 [SMILES: N#CCN1CCN(c2nccnc2Nc2ccc(C(F)(F)F)cc2)CC1]

Following the general procedure in scheme 2, 2-bromoacetonitrile was used. Offered 2-(4-(3-((4-(trifluoromethyl)phenyl)amino)pyrazin-2-yl)piperazin-1-yl)acetonitrile as white solid. (Positive, ES, m/z): 363.26. ¹H NMR (400 MHz, DMSO) δ 8.59 (s, 1H), 7.93-7.78 (m, 4H), 7.63 (d, J=8.5 Hz, 2H), 3.81 (s, 2H), 3.21 (s, 4H), 2.73 (t, J=4.8 Hz, 4H).

### Example 176. Synthesis of 2-methyl-2-(4-(3-((4-(trifluoromethyl)phenyl)amino)pyrazin-2-yl)piperazin-1-yl)propanenitrile

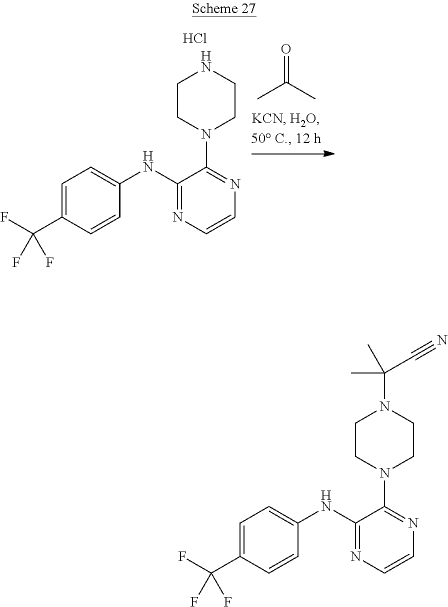

A mixture of 3-(piperazin-1-yl)-N-[4-(trifluoromethyl)phenyl]pyrazin-2-amine (200 mg, 0.619 mmol, 1 equiv) and acetone (71.85 mg, 1.238 mmol, 2 equiv), KCN (80.56 mg, 1.238 mmol, 2 equiv) in H₂O (2 mL) was stirred for 12 h at 50° C. under nitrogen atmosphere. The resulting mixture was extracted with EtOAc (3×5 mL). The combined organic layers were washed with water (lx 10 mL), dried over anhydrous Na₂SO₄. After filtration, the filtrate was concentrated under reduced pressure. This resulted in 2-methyl-2-[4-(3-{[4-(trifluoromethyl)phenyl]amino}pyrazin-2-yl)piperazin-1-yl]propanenitrile (150 mg, 59.01%) as a brown solid. LCMS:(ES, m/z): [M+H]⁺=391. ¹H NMR (400 MHz, DMSO-d₆) δ 8.61 (s, 1H), 7.94-7.81 (m, 4H), 7.62 (d, J=8.6 Hz, 2H), 3.26-3.15 (m, 4H), 2.84-2.78 (m, 4H), 1.49 (s, 6H).

### Example 177: Synthesis of 2-methyl-1-(4-(3-((6-(trifluoromethyl)pyridin-3-yl)amino)pyrazin-2-yl)piperazin-1-yl)prop-2-en-1-one

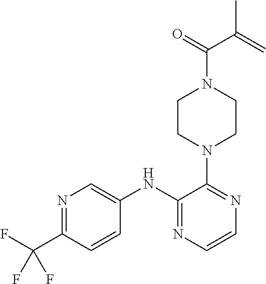 [SMILES: C=C(C)C(=O)N1CCN(c2nccnc2Nc2ccc(C(F)(F)F)nc2)CC1]

Following the general procedure in scheme 1, methacryloyl chloride was used in step 4. Offered 2-methyl-1-(4-(3-((6-(trifluoromethyl)pyridin-3-yl)amino)pyrazin-2-yl)piperazin-1-yl)prop-2-en-1-one as white solid. (Positive, ES, m/z): 393.52. ¹H NMR (400 MHz, DMSO) δ 9.03 (d, J=2.5 Hz, 1H), 8.86 (s, 1H), 8.39 (dd, J=8.7, 2.5 Hz, 1H), 7.90 (q, J=2.8 Hz, 2H), 7.83 (d, J=8.7 Hz, 1H), 5.25-5.19 (m, 1H), 5.06-4.99 (m, 1H), 3.72 (s, 4H), 3.17 (t, J=5.1 Hz, 4H), 1.89 (d, J=1.3 Hz, 3H).

### Example 178: Synthesis of (E)-1-(4-(3-((6-(trifluoromethyl)pyridin-3-yl)amino)pyrazin-2-yl)piperazin-1-yl)but-2-en-1-one

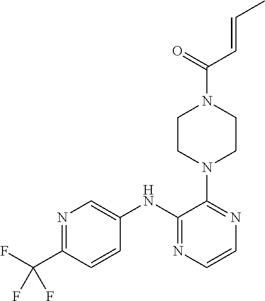 [SMILES: C/C=C/C(=O)N1CCN(c2nccnc2Nc2ccc(C(F)(F)F)nc2)CC1]

Following the general procedure in scheme 1, (E)-but-2-enoyl chloride was used in step 4. Offered (E)-1-(4-(3-((6-(trifluoromethyl)pyridin-3-yl)amino)pyrazin-2-yl)piperazin-1-yl)but-2-en-1-one as white solid. (Positive, ES, m/z): 393.48. ¹H NMR (400 MHz, DMSO) δ 9.03 (d, J=2.5 Hz, 1H), 8.87 (s, 1H), 8.39 (dd, J=8.6, 2.5 Hz, 1H), 7.90 (s, 2H), 7.83 (d, J=8.7 Hz, 1H), 6.80-6.65 (m, 1H), 6.56 (dd, J=15.0, 1.7 Hz, 1H), 3.77 (s, 4H), 3.17 (s, 4H), 1.86 (dd, J=6.8, 1.5 Hz, 3H).

### Example 179. Synthesis of (E)-4-(methyl(prop-2-yn-1-yl)amino)-1-(4-(3-((4-(trifluoromethyl)phenyl)amino)pyrazin-2-yl)piperazin-1-yl)but-2-en-1-one

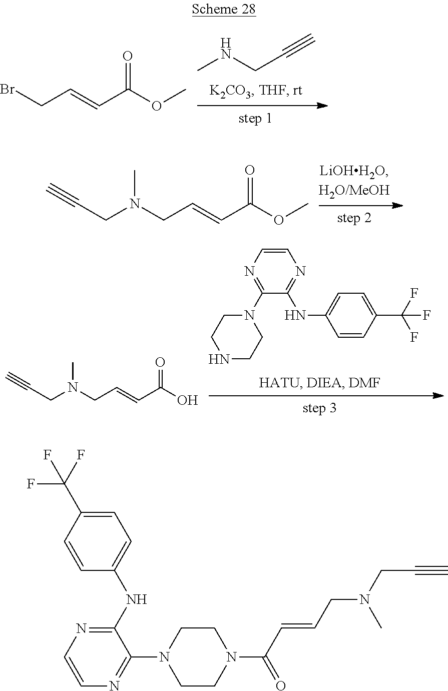

### Step 1: Synthesis of afford methyl (2E)-4-[methyl(prop-2-yn-1-yl)amino]but-2-enoate

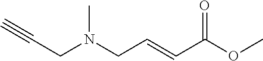

Into a 40 mL vessel was placed a solution of methyl (2E)-4-bromobut-2-enoate (900 mg, 5.02 mmol, 1.00 equiv) 2-propyn-1-amine, N-methyl-(382.18 mg, 5.53 mmol, 1.10 equiv) and K₂CO₃ (2084.51 mg, 15.08 mmol, 3.00 equiv) in THE (10 mL) was stirred for 10 h at room temperature. The resulting mixture was extracted with EtOAc (2×50 mL). The combined organic layers were washed with brine (1×30 mL), dried over anhydrous Na₂SO₄. After filtration, the filtrate was concentrated under reduced pressure, the residue was purified by silica gel column chromatography, eluted with PE/EA (5:1) to afford methyl (2E)-4-[methyl(prop-2-yn-1-yl)amino]but-2-enoate (700 mg, 74.94%) as a yellow liquid. LCMS:(ES, m/z): [M+H]⁺=168

### Step 2: Synthesis of (E)-4-(methyl(prop-2-yn-1-yl)amino)but-2-enoic acid

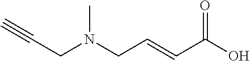

A solution of methyl (2E)-4-[methyl(prop-2-yn-1-yl)amino]but-2-enoate (640 mg, 3.82 mmol, 1.00 equiv) and LiOH·H₂O (275.01 mg, 11.48 mmol, 3.00 equiv) in H₂O (2 mL) and MeOH (8 mL) was stirred for 7 h at room temperature. The resulting mixture was concentrated under reduced pressure. The crude product was used in the step directly without further purification. LCMS:(ES, m/z): [M+H]⁺=154.

### Step 3: Synthesis of (E)-4-(methyl(prop-2-yn-1-yl)amino)-1-(4-(3-((4-(trifluoromethyl)phenyl)amino)pyrazin-2-yl)piperazin-1-yl)but-2-en-1-one

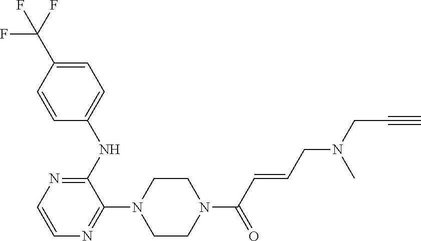 [SMILES: C#CCN(C)C/C=C/C(=O)N1CCN(c2nccnc2Nc2ccc(C(F)(F)F)cc2)CC1]

A solution of (2E)-4-[methyl(prop-2-yn-1-yl)amino]but-2-enoic acid (300 mg, 1.95 mmol, 1.00 equiv),3-(piperazin-1-yl)-N-[4-(trifluoromethyl)phenyl]pyrazin-2-amine (633.22 mg, 1.95 mmol, 1.00 equiv), HATU (1042.55 mg, 2.74 mmol, 1.40 equiv) and DIEA (759.38 mg, 5.87 mmol, 3.00 equiv) in DMF (10 mL, 129.21 mmol, 65.98 equiv) was stirred for 7 h at room temperature. The reaction was quenched with 10 Ml water and then extracted with EtOAc (50×3 mL). The combined organic layers were washed with brine (1×30 mL), dried over anhydrous Na₂SO₄, concentrated under vacuum. The crude product was purified by reverse phase flash with the following conditions (Acetonitrile: Water/0.05% ammonia water 20%-60% 18 min) to afford methy (2E)-4-[methyl(prop-2-yn-1-yl)amino]-1-[4-(3-{[4-(trifluoromethyl)phenyl]amino}pyrazin-2-yl)piperazin-1-yl]but-2-en-1-one (220 mg, 24.50%) as a yellow solid. LCMS:(ES, m/z): [M+H]⁺=459. ¹H NMR (400 MHz, CDCl₃-d₆): δ7.98-7.92 (s, 1H), 7.86-7.79 (m, 1H), 7.79-7.73 (m, 2H), 7.65-7.59 (m, 2H), 7.31 (m, 1H), 6.95-6.84 (m, 1H), 6.61-6.53 (m, 1H), 3.92-3.87 (m, 4H), 3.44-3.39 (m, 2H), 3.33-3.27 (m, 2H), 3.22-3.17 (m, 4H), 2.40 (s, 3H), 2.32-2.27 (s, 1H).

### Example 180: Synthesis of 2,3-dihydroxy-1-(4-(3-((4-(trifluoromethyl)phenyl)amino)pyrazin-2-yl)piperazin-1-yl)propan-1-one

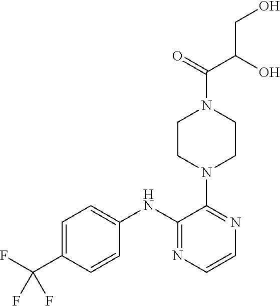 [SMILES: O=C(C(O)CO)N1CCN(c2nccnc2Nc2ccc(C(F)(F)F)cc2)CC1]

Following the general procedure in scheme 10, 2,3-dihydroxypropanoic acid was used. Offered 2,3-dihydroxy-1-(4-(3-((4-(trifluoromethyl)phenyl)amino)pyrazin-2-yl)piperazin-1-yl)propan-1-one as white solid. (Positive, ES, m/z): 412.31. ¹H NMR (400 MHz, DMSO) δ 8.62 (s, 1H), 7.98-7.79 (m, 4H), 7.65 (d, J=8.5 Hz, 2H), 4.99 (d, J=7.3 Hz, 1H), 4.75 (t, J=5.8 Hz, 1H), 4.39 (dt, J=7.0, 5.6 Hz, 1H), 3.87-3.66 (m, 4H), 3.60-3.44 (m, 2H), 3.14 (d, J=14.9 Hz, 4H).

### Synthesis of (E)-4-oxo-4-(4-(3-((4-(trifluoromethyl)phenyl)amino)pyrazin-2-yl)piperazin-1-yl)but-2-enenitrile (Example 181) and (Z)-4-oxo-4-(4-(3-((4-(trifluoromethyl)phenyl)amino)pyrazin-2-yl)piperazin-1-yl)but-2-enenitrile (Example 182)

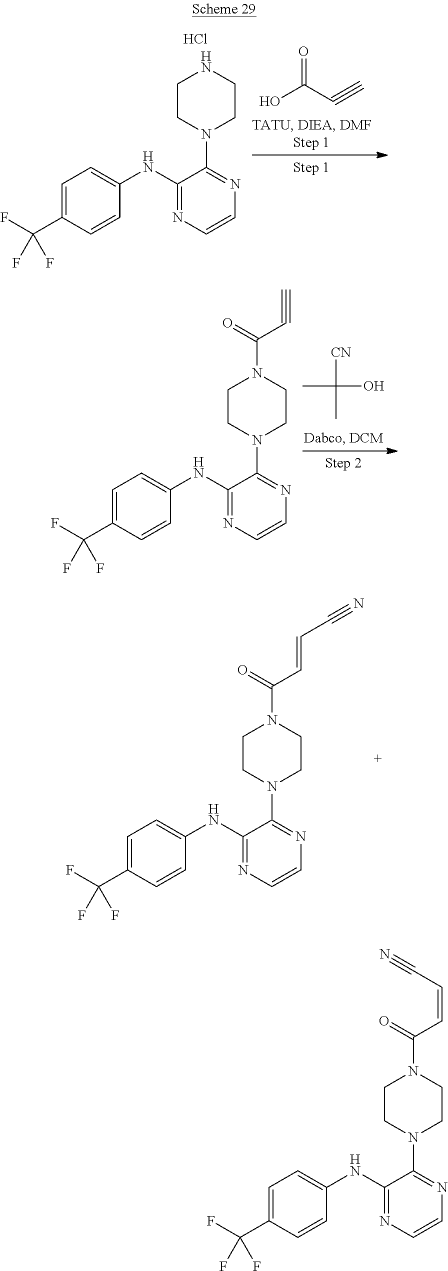

### Step 1: Synthesis of 1-(4-(3-((4-(trifluoromethyl)phenyl)amino)pyrazin-2-yl)piperazin-1-yl)prop-2-yn-1-one

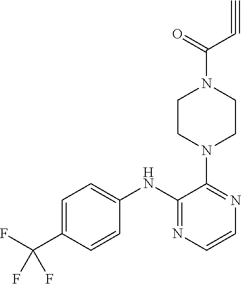 [SMILES: C#CC(=O)N1CCN(c2nccnc2Nc2ccc(C(F)(F)F)cc2)CC1]

A mixture of 3-(piperazin-1-yl)-N-[4-(trifluoromethyl)phenyl]pyrazin-2-amine (200 mg, 0.619 mmol, 1 equiv), propiolic acid (56.33 mg, 0.805 mmol, 1.3 equiv), DIlEA (239.85 mg, 1.857 mmol, 3 equiv) and HATU (282.25 mg, 0.743 mmol, 1.2 equiv) in DMF (2 mL) was stirred for 2 h at room temperature under nitrogen atmosphere. The resulting mixture was diluted with water (10 mL). The resulting mixture was extracted with EtOAc (3×10 mL). The combined organic layers were washed with dried over anhydrous Na₂SO₄. After filtration, the filtrate was concentrated under reduced pressure. The residue was purified by silica gel column chromatography, eluted with PE/EA (1:1) to afford 1-[4-(3-{[4-(trifluoromethyl)phenyl]amino}pyrazin-2-yl)piperazin-1-yl]prop-2-yn-1-one (120 mg, 49.90%) as a white solid.

### Step 2: Synthesis of (E)-4-oxo-4-(4-(3-((4-(trifluoromethyl)phenyl)amino)pyrazin-2-yl)piperazin-1-yl)but-2-enenitrile (Example 181) and (Z)-4-oxo-4-(4-(3-((4-(trifluoromethyl)phenyl)amino)pyrazin-2-yl)piperazin-1-yl)but-2-enenitrile (Example 182)

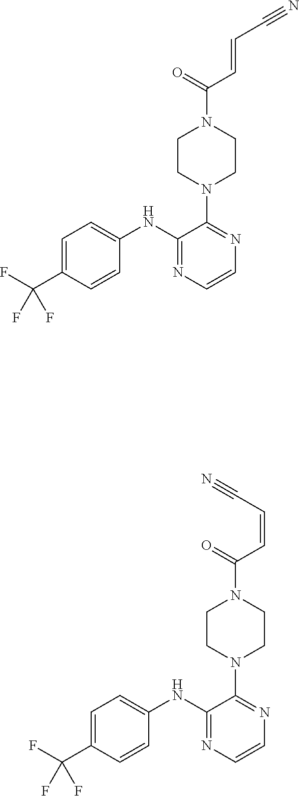 [SMILES: N#C/C=C/C(=O)N1CCN(c2nccnc2Nc2ccc(C(F)(F)F)cc2)CC1 ; N#C/C=C\C(=O)N1CCN(c2nccnc2Nc2ccc(C(F)(F)F)cc2)CC1]

A mixture of 1-[4-(3-{[4-(trifluoromethyl)phenyl]amino}pyrazin-2-yl)piperazin-1-yl]prop-2-yn-1-one (120 mg, 0.246 mmol, 1 equiv) and acetone cyanohydrin (25.14 mg, 0.295 mmol, 1.20 equiv), Dabco (5.52 mg, 0.049 mmol, 0.2 equiv) in DCM (2 mL) was stirred for 4 h at room temperature under nitrogen atmosphere. The resulting mixture was diluted with water (10 mL). The resulting mixture was extracted with EtOAc (3×10 mL). The combined organic layers were washed with dried over anhydrous Na₂SO₄. After filtration, the filtrate was concentrated under reduced pressure. The crude product (300 mg) was purified by Prep-HPLC with the following conditions (Column: Welch Xtimate C18 ExRS, 250 mm, 10 m; Mobile Phase A: Water (0.05% NH₃·H₂O), Mobile Phase B: ACN; Flow rate: 90 mL/min; Gradient: 90% B to 90% B in 10 min, 90% B; Wave Length: 254 nm; RT1(min): 7; Number Of Runs: 5) to afford (E)-4-oxo-4-(4-(3-((4-(trifluoromethyl)phenyl)amino)pyrazin-2-yl)piperazin-1-yl)but-2-enenitrile (80 mg, 62.2% as white solid.LCMS:(ES, m/z): [M+H]⁺=403.1H NMR (400 MHz, DMSO-d6) δ 8.62 (s, 1H), 7.95-7.87 (m, 3H), 7.87-7.77 (m, 2H), 7.64 (d, J=8.6 Hz, 2H), 6.57 (d, 0.1=15.8 Hz, 1H), 3.88-3.75 (m, 4H), 3.21-3.13 (m, 4H). (Z)-4-oxo-4-(4-(3-((4-(trifluoromethyl)phenyl)amino)pyrazin-2-yl)piperazin-1-yl)but-2-enenitrile (40 mg, 31.1%). LCMS: [M+H]⁺=403. ¹H NMR (400 MHz, DMSO-d₆) δ 8.62 (s, 1H), 7.95-7.86 (m, 3H), 7.84 (d, J=2.8 Hz, 1H), 7.64 (d, J=8.6 Hz, 2H), 7.43 (d, J=11.6 Hz, 1H), 6.21 (d, J=11.6 Hz, 1H), 3.82-3.76 (m, 2H), 3.76-3.69 (m, 2H), 3.22-3.13 (m, 4H).

### Example 183: Synthesis of (E)-4-oxo-4-(4-(3-((6-(trifluoromethyl)pyridin-3-yl)amino)pyrazin-2-yl)piperazin-1-yl)but-2-enenitrile

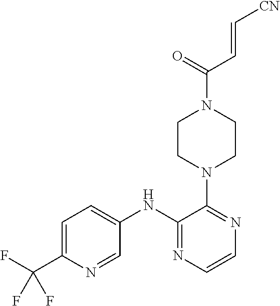 [SMILES: N#C/C=C/C(=O)N1CCN(c2nccnc2Nc2ccc(C(F)(F)F)nc2)CC1]

Following the general procedure in scheme 27, 3-(piperazin-1-yl)-N-(6-(trifluoromethyl)pyridin-3-yl)pyrazin-2-amine was used in step 1. Offered (E)-4-oxo-4-(4-(3-((6-(trifluoromethyl)pyridin-3-yl)amino)pyrazin-2-yl)piperazin-1-yl)but-2-enenitrile as white solid. (Positive, ES, m/z): 404.1. ¹H NMR (400 MHz, DMSO) δ 9.03 (d, J=2.5 Hz, 1H), 8.89 (s, 1H), 8.39 (d, J=8.4 Hz, 1H), 7.94-7.76 (m, 3H), 6.59 (d, J=15.8 Hz, 1H), 3.92-3.69 (m, 4H), 3.21 (d, J=5.2 Hz, 4H).

### Example 184: Synthesis of (Z)-4-oxo-4-(4-(3-((6-(trifluoromethyl)pyridin-3-yl)amino)pyrazin-2-yl)piperazin-1-yl)but-2-enenitrile

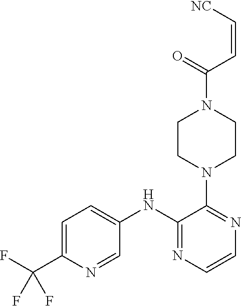 [SMILES: N#C/C=C\C(=O)N1CCN(c2nccnc2Nc2ccc(C(F)(F)F)nc2)CC1]

Following the general procedure in scheme 27, 3-(piperazin-1-yl)-N-(6-(trifluoromethyl)pyridin-3-yl)pyrazin-2-amine was used in step 1. Offered (Z)-4-oxo-4-(4-(3-((6-(trifluoromethyl)pyridin-3-yl)amino)pyrazin-2-yl)piperazin-1-yl)but-2-enenitrile as white solid. (Positive, ES, m/z): 404.1. ¹H NMR (400 MHz, DMSO) δ 9.04 (d, J=2.5 Hz, 1H), 8.89 (s, 1H), 8.40 (dd, J=8.7, 2.5 Hz, 1H), 7.91 (q, J=2.8 Hz, 2H), 7.84 (d, J=8.7 Hz, 1H), 7.45 (d, J=11.7 Hz, 1H), 6.23 (d, J=11.6 Hz, 1H), 3.84-3.62 (m, 4H), 3.22 (q, J=5.5 Hz, 4H).

### Example 185. Synthesis of N-cyano-N-methyl-4-(3-((4-(trifluoromethyl)phenyl)amino)pyrazin-2-yl)piperazine-1-carboxamide

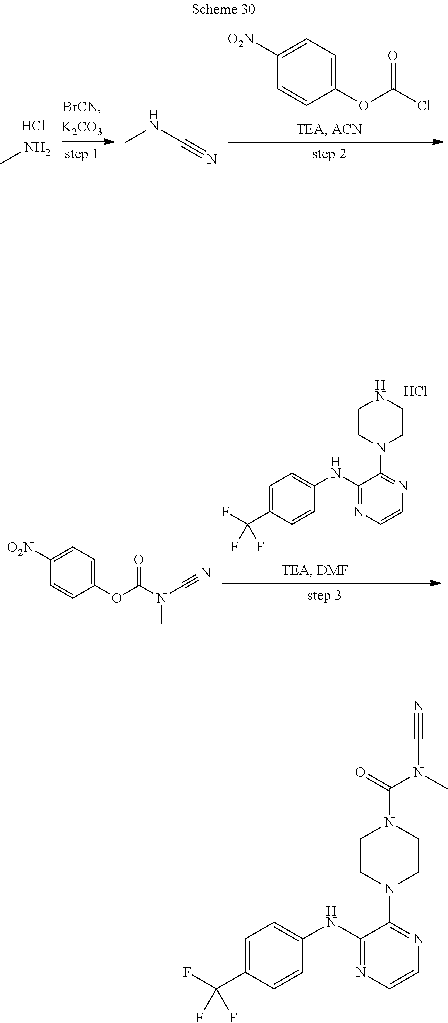

### Step 1: Synthesis of N-methylcyanamide

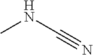

To a solution of methanamine hydrochloride (2.0 g, 29.62 mmol, 1.00 eq.) and cyanic bromide (3.14 g, 29.62 mmol, 1.00 eq.) in acetonitrile (30 mL) was added potassium carbonate (12.28 g, 88.87 mmol, 3.00 eq.) at 0° C. The reaction mixture was stirred at 20° C. for 3 hrs before filtration. The filtrate was concentrated under reduced pressure to afford N-methylcyanamide (1.65 g, 99.3%) as a colorless oil.

### Step 2: Synthesis of 4-nitrophenyl N-cyano-N-methylcarbamate

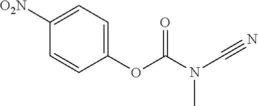 [SMILES: CN(C#N)C(=O)Oc1ccc([N+](=O)[O-])cc1]

To a solution of N-methylcyanamide (1.65 g, 29.43 mmol, 1.00 eq.) and 4-nitrophenyl 2-iodoacetate (8.98 g, 29.43 mmol, 1.00 eq.) in acetonitrile (30 mL) was added triethylamine (8.93 g, 88.29 mmol, 3.00 eq.) at 0° C. The reaction mixture was stirred at 20° C. for 12 hrs. The resulting mixture was diluted with 20 mL H₂O and extracted with ethyl acetate (60 mL×3). The combined organic layers were washed with brine (20.00 mL), dried over anhydrous Na₂SO₄. The filtrate was concentrated under reduced pressure to afford 4-nitrophenyl N-cyano-N-methylcarbamate (5.0 g, 76.82%) as a yellow solid.

### Step 3: Synthesis of N-cyano-N-methyl-4-(3-((4-(trifluoromethyl)phenyl)amino)pyrazin-2-yl)piperazine-1-carboxamide

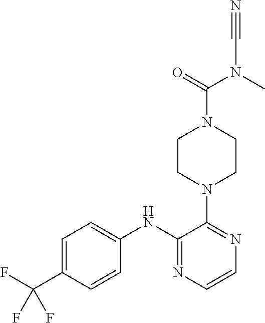 [SMILES: CN(C#N)C(=O)N1CCN(c2nccnc2Nc2ccc(C(F)(F)F)cc2)CC1]

To a solution of 3-(piperazin-1-yl)-N-(4-(trifluoromethyl)phenyl) pyrazin-2-amine (120 mg, 0.37 mmol, 1.00 eq.) and 4-nitrophenyl N-cyano-N-methylcarbamate (164 mg, 0.74 mmol, 2.00 eq.) in DMF (3 mL) was added triethylamine (75.11 mg, 0.74 mmol, 2.00 eq.) at 0° C. The reaction mixture was stirred at 20° C. for 3 hrs. The reaction was monitored by LCMS. The resulting mixture was diluted with 20 mL H₂O and extracted with ethyl acetate (60 mL×3). The combined organic layers were washed with brine (20.00 mL), dried over anhydrous Na₂SO₄. The residue was purified by silica gel column chromatography, eluted with PE: THE (1:1) first. The crude product was purified by Prep-HPLC with the following conditions (Column: YMC-Actus Triart C18, 250*50 mm, 10 m; Mobile Phase A: 0.1% NH₃H₂O, Mobile Phase B: ACN; Flow rate: 80 mL/min; Gradient: 13% B-50% B-17 min) to afford N-cyano-N-methyl-4-(3-((4-(trifluoromethyl)phenyl)amino) pyrazin-2-yl) piperazine-1-carboxamide (500 mg, 40.61%) as a white solid. After filtration, the filtrate was concentrated under reduced pressure. The residue was purified by silica gel column chromatography, eluted with PE: THF (1:1) to afford N-cyano-N-methyl-4-(3-((4-(trifluoromethyl)phenyl)amino) pyrazin-2-yl) piperazine-1-carboxamide (40 mg, 26.59%) as white solid. LCMS:(ES, m/z): [M+H]⁺=406. iH NMR (300 MHz, DMSO-d₆) δ 8.60 (s, 1H), 7.97-7.80 (m, 4H), 7.64 (d, J=8.4 Hz, 2H), 3.80-3.61 (m, 4H), 3.25-3.16 (m, 4H), 3.12 (s, 3H).

### Example 186: Synthesis of 2-(hydroxymethyl)-1-(4-(3-((4-(trifluoromethyl)phenyl)amino)pyrazin-2-yl)piperazin-1-yl)prop-2-en-1-one

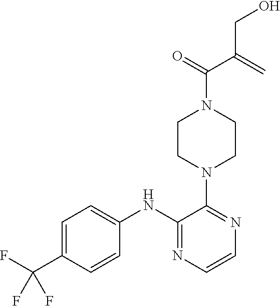 [SMILES: C=C(CO)C(=O)N1CCN(c2nccnc2Nc2ccc(C(F)(F)F)cc2)CC1]

Following the general procedure in scheme 10, 2-(hydroxymethyl)acrylic acid was used. Offered 2-(hydroxymethyl)-1-(4-(3-((4-(trifluoromethyl)phenyl)amino)pyrazin-2-yl)piperazin-1-yl)prop-2-en-1-one as white solid. (Positive, ES, m/z): 408.27. ¹H NMR (400 MHz, DMSO) δ 8.61 (s, 1H), 7.96-7.87 (m, 3H), 7.84 (d, 0.1=2.8 Hz, 1H), 7.65 (d, I=8.6 Hz, 2H), 5.37 (q, J=1.6 Hz, 1H), 5.14-5.08 (m, 2H), 4.09 (dt, J=5.8, 1.5 Hz, 2H), 3.75 (t, J=4.9 Hz, 4H), 3.14 (t, J=5.1 Hz, 4H).

### Example 187: Synthesis of 2-(4-(3-((4-(trifluoromethyl)phenyl)amino)pyrazin-2-yl)piperazine-1-carbonyl)allyl acetate

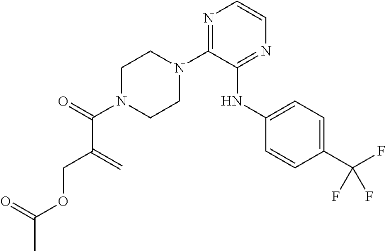 [SMILES: C=C(COC(C)=O)C(=O)N1CCN(c2nccnc2Nc2ccc(C(F)(F)F)cc2)CC1]

Following the general procedure in scheme 1, acetyl chloride was used in step 4. Offered 2-(4-(3-((4-(trifluoromethyl)phenyl)amino)pyrazin-2-yl)piperazine-1-carbonyl)allyl acetate as white solid. (Positive, ES, m/z): 450.3. ¹H NMR (400 MHz, DMSO) δ 8.62 (s, 1H), 7.96-7.86 (m, 3H), 7.85 (d, J=2.8 Hz, 1H), 7.65 (d, J=8.6 Hz, 2H), 5.54 (s, 1H), 5.33 (s, 1H), 4.71 (s, 2H), 3.76 (s, 4H), 3.15 (t, J=5.0 Hz, 4H).

### Example 188: Synthesis of 2-(fluoromethyl)-1-(4-(3-((4-(trifluoromethyl)phenyl)amino)pyrazin-2-yl)piperazin-1-yl)prop-2-en-1-one

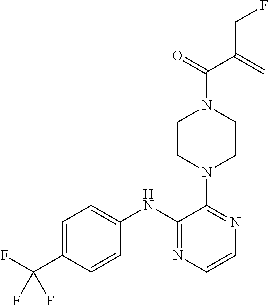 [SMILES: C=C(CF)C(=O)N1CCN(c2nccnc2Nc2ccc(C(F)(F)F)cc2)CC1]

Following the general procedure in scheme 10, 2-(fluoromethyl)acrylic acid was used. Offered 2-(fluoromethyl)-1-(4-(3-((4-(trifluoromethyl)phenyl)amino)pyrazin-2-yl)piperazin-1-yl)prop-2-en-1-one as white solid. (Positive, ES, m/z): 410.21. ¹H NMR (400 MHz, DMSO) δ 8.62 (s, 1H), 7.98-7.87 (m, 3H), 7.84 (d, J=2.7 Hz, 1H), 7.65 (d, J=8.6 Hz, 2H), 5.65 (d, J=3.9 Hz, 1H), 5.43 (s, 1H), 5.14 (s, 1H), 5.03 (s, 1H), 3.78 (s, 4H), 3.15 (t, J=5.0 Hz, 4H).

### Example 189: Synthesis of 2-(pyrrolidin-1-ylmethyl)-1-(4-(3-((4-(trifluoromethyl)phenyl)amino)pyrazin-2-yl)piperazin-1-yl)prop-2-en-1-one

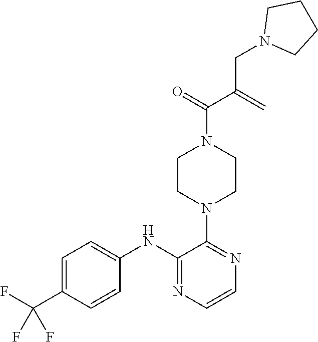 [SMILES: C=C(CN1CCCC1)C(=O)N1CCN(c2nccnc2Nc2ccc(C(F)(F)F)cc2)CC1]

Following the general procedure in scheme 10, 2-(pyrrolidin-1-ylmethyl)acrylic acid was used. Offered 2-(pyrrolidin-1-ylmethyl)-1-(4-(3-((4-(trifluoromethyl)phenyl)amino)pyrazin-2-yl)piperazin-1-yl)prop-2-en-1-one as white solid. (Positive, ES, m/z): 461.35. ¹H NMR (400 MHz, DMSO) δ 9.53 (s, 1H), 8.64 (d, J=13.8 Hz, 1H), 7.97-7.80 (m, 4H), 7.65 (t, J=7.7 Hz, 2H), 5.99-5.65 (m, 1H), 4.06-3.64 (m, 4H), 3.24-3.13 (m, 4H), 2.91-2.57 (m, 4H), 2.08 (s, 5H), 1.34-1.19 (m, 1H).

### Example 190: Synthesis of 2-(trifluoromethoxy)-1-(4-(3-((4-(trifluoromethyl)phenyl)amino)pyrazin-2-yl)piperazin-1-yl)ethan-1-one

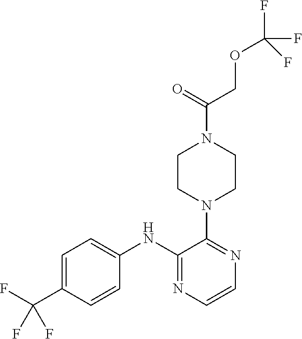 [SMILES: O=C(COC(F)(F)F)N1CCN(c2nccnc2Nc2ccc(C(F)(F)F)cc2)CC1]

Following the general procedure in scheme 10, 2-(trifluoromethoxy)acetic acid was used. Offered 2-(trifluoromethoxy)-1-(4-(3-((4-(trifluoromethyl)phenyl)amino)pyrazin-2-yl)piperazin-1-yl)ethan-1-one as white solid. (Positive, ES, m/z): 450.41. ¹H NMR (400 MHz, DMSO) δ 8.63 (s, 1H), 7.96-7.80 (m, 4H), 7.65 (d, J=8.6 Hz, 2H), 5.00 (s, 2H), 3.71 (s, 2H), 3.58 (s, 2H), 3.23-3.06 (m, 4H).

### Example 191. Synthesis of N-methyl-N-(2-((3-((4-(trifluoromethyl)phenyl)amino)pyrazin-2-yl)oxy)ethyl)acrylamide

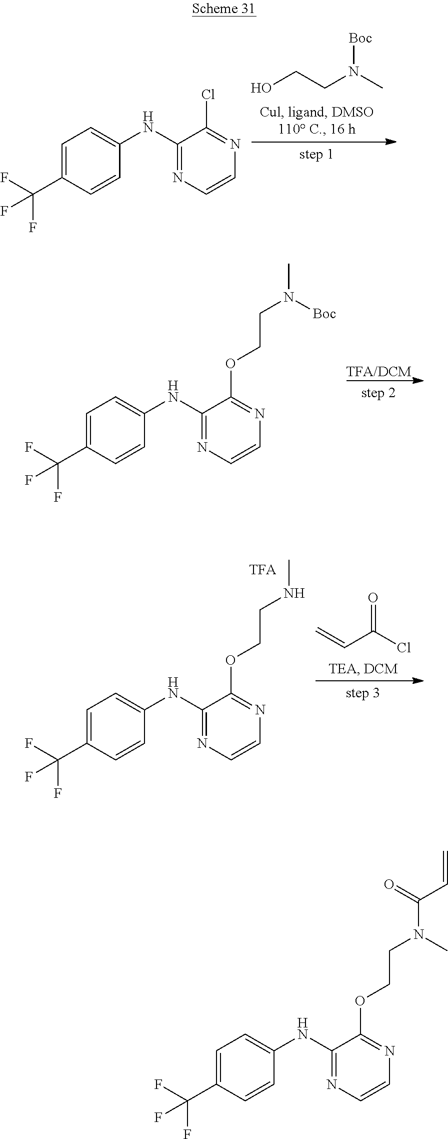

### Step 1: Synthesis of tert-butyl N-Methyl-N-{2-[(3-{[4-(trifluoromethyl)phenyl]amino}pyrazin-2-yl)oxy]ethyl}carbamate

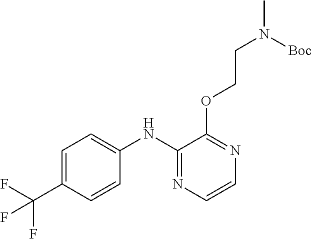 [SMILES: CN(CCOc1nccnc1Nc1ccc(C(F)(F)F)cc1)C(=O)OC(C)(C)C]

Into a 40 mL vial were added 3-chloro-N-[4-(trifluoromethyl)phenyl]pyrazin-2-amine (600 mg, 2.19 mmol, 1 equiv), CuI (300.67 mg, 1.57 mmol, 0.72 equiv), Cs₂CO₃ (3579.21 mg, 10.987 mmol, 5.01 equiv), 2,2,6,6-tetramethylheptane-3,5-dione (60.61 mg, 0.32 mmol, 0.15 equiv), tert-butyl N-(2-hydroxyethyl)-N-methylcarbamate (1767.37 mg, 10.08 mmol, 4.60 equiv) and DMSO (6 mL) at rt. The mixture was stirred for 8 h at 110° C. under nitrogen atmosphere. Desired product could be detected by LCMS. The resulting mixture was diluted with water (30 mL); The resulting mixture was extracted with EtOAc (30mLx3) dried over anhydrous Na₂SO₄. After filtration, the filtrate was concentrated under reduced pressure. The residue was purified by silica gel column chromatography, eluted with PE/EA (1:1) to afford tert-butyl N-Methyl-N-{2-[(3-{[4-(trifluoromethyl)phenyl]amino}pyrazin-2-yl)oxy]ethyl}carbamate (220 mg, 26.54%) as a yellow oil.

### Step 2: Synthesis of 3-(2-(methylamino)ethoxy)-N-(4-(trifluoromethyl)phenyl)pyrazin-2-amine

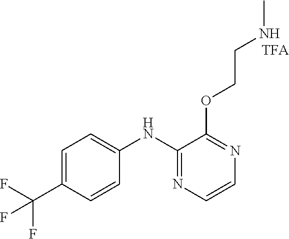 [SMILES: CNCCOc1nccnc1Nc1ccc(C(F)(F)F)cc1]

Into a 40 mL vial were added tert-butyl N-Methyl-N-{2-[(3-{[4-(trifluoromethyl)phenyl]amino}pyrazin-2-yl)oxy]ethyl}carbamate (220 mg, 0.53 mmol, 1 equiv), TFA (1 mL, 1.53 mmol, 55.52 equiv) and DCM (3 mL) at rt. Desired product could be detected by LCMS. The resulting mixture was concentrated under reduced pressure. The crude resulting mixture was used in the next step directly without further purification.

### Step 3: Synthesis of N-Methyl-N-{2-[(3-{[4-(trifluoromethyl)phenyl]amino}pyrazin-2-yl)oxy]ethyl}prop-2-enamide

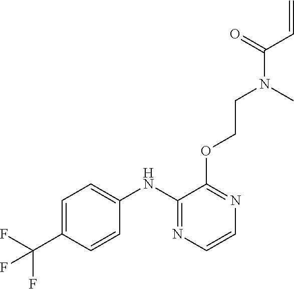 [SMILES: C=CC(=O)N(C)CCOc1nccnc1Nc1ccc(C(F)(F)F)cc1]

A solution of 3-[2-(methylamino) ethoxy]-N-[4-(trifluoromethyl)phenyl]pyrazin-2-amine (0.27 g, 0.63 mmol, 1 equiv) in DCM (5 ml) was treated with TFA (0.17 g, 1.68 mmol, 2.65 equiv) for 5 min at 0° C. under nitrogen atmosphere followed by the addition of prop-2-enoyl chloride (0.06 g, 0.65 mmol, 1.04 equiv) dropwise at 0° C. Desired products could be detected by LCMS. The resulting mixture was diluted with water (30 mL). The resulting mixture was extracted with CH₂Cl₂ (3×30 mL). The combined organic layers were washed with water (2×30 mL), dried over anhydrous Na₂SO₄. After filtration, the filtrate was concentrated under reduced pressure. The crude product was purified by reverse phase flash with the following conditions (Acetonitrile: Water/0.05% ammonia water 25%-67% 18 min) to afford N-Methyl-N-{2-[(3-{[4-(trifluoromethyl)phenyl]amino}pyrazin-2-yl)oxy]ethyl}prop-2-enamide (65 mg, 28.2%) as a white solid. LCMS:(ES, m/z): [M+H]⁺=367. ¹H NMR (400 MHz, DMSO-d₆) δ 8.79 (d, J=11.2 Hz, 1H), 8.10 (d, J=8.8 Hz, 1H), 8.04 (d, J=8.8 Hz, 1H), 7.78 (t, J=3.4 Hz, 1H), 7.67 (d, J=4.2 Hz, 2H), 7.60 (d, J=7.7, 3.1 Hz, 1H), 6.83 (d, J=16.8 Hz, 1H), 6.10 (d, J=16.8 Hz, 1H), 5.63 (d, J=10.4 Hz, 1H), 4.68-4.52 (m, 2H), 3.87 (d, J=5.2 Hz, 2H), 3.06 (d, J=75.2 Hz, 3H).

### Example 192. Synthesis of N-(2-((3-((4-(trifluoromethyl)phenyl)amino)pyrazin-2-yl)oxy)ethyl)acrylamide

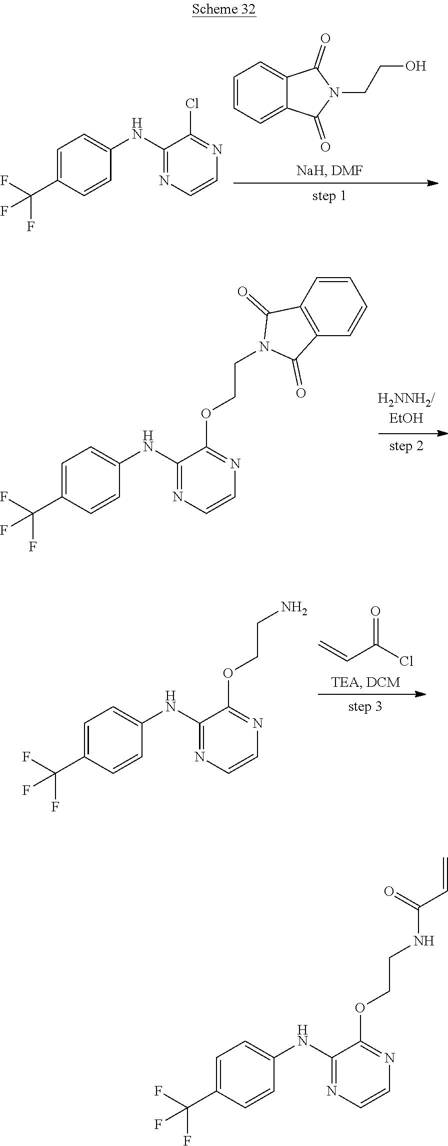

### Step 1: Synthesis of 2-{2-[(3-{[4-(trifluoromethyl)phenyl]amino}pyrazin-2-yl)oxy]ethyl}isoindole-1,3-dione

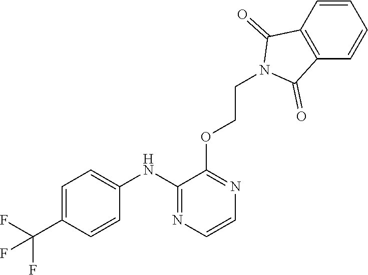 [SMILES: O=C1c2ccccc2C(=O)N1CCOc1nccnc1Nc1ccc(C(F)(F)F)cc1]

Into a 40 mL vial were added 3-chloro-N-[4-(trifluoromethyl)phenyl]pyrazin-2-amine (500 mg, 1.83 mmol, 1 equip) CuI (250.56 mg, 1.31 mmol, 0.72 equiv), Cs₂CO₃ (1786.01 mg, 5.41 mmol, 3 equiv), 2,2,6,6-tetram ethylheptane-3,5-di one (50.51 mg, 0.27 mmol, 0.15 equiv), 2-(2-hydroxyethyl)-2,3-dihydro-1H-isoindole-1,3-dione (1048 mg, 5.41 mmol, 3 equiv) and DMSO (5 mL) at room temperature. The resulting mixture was stirred for 2 h at 110° C. under nitrogen atmosphere. Desired product could be detected by LCMS. The resulting mixture was diluted with water (50 mL). The resulting mixture was extracted with EtOAc (3×50 mL). The combined organic layers were washed with water (3×50 mL), dried over anhydrous Na₂SO₄. After filtration, the filtrate was concentrated under reduced pressure. The residue was purified by silica gel column chromatography, eluted with PE/EA (1:1) to afford 2-{2-[(3-{[4-(trifluoromethyl)phenyl]amino}pyrazin-2-yl)oxy]ethyl}isoindole-1,3-dione (450 mg 57.49%) as a yellow solid.

### Step 2: Synthesis of 3-(2-aminoethoxy)-N-(4-(trifluoromethyl)phenyl)pyrazin-2-amine

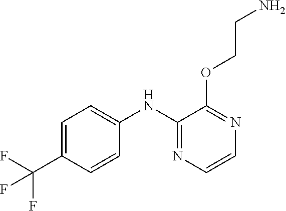 [SMILES: NCCOc1nccnc1Nc1ccc(C(F)(F)F)cc1]

Into a 40 mL vial were added 2-{2-[(3-{[4-(trifluoromethyl)phenyl]amino}pyrazin-2-yl)oxy]ethyl}isoindole-1,3-dione (400 mg,0.93 mmol, 1 equiv), EtOH (5 mL) and hydrazine hydrate (85%) (163.61 mg, 3.269 mmol, 3.5 equiv) at room temperature. The resulting mixture was stirred for 3 h at 80° C. Desired product could be detected by LCMS. The resulting mixture was diluted with water (50 mL). The resulting mixture was extracted with EtOAc (3×20). The combined organic layers were washed with H₂O (2×20 mL), dried over anhydrous Na₂SO₄. After filtration, the filtrate was concentrated under reduced pressure. The crude resulting mixture was used in the next step directly without further purification.

### Step 3: Synthesis of N-{2-[(3-{[4-(trifluoromethyl)phenyl]amino}pyrazin-2-yl)oxy]ethyl}prop-2-enamide

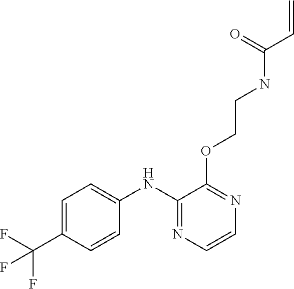 [SMILES: C=CC(=O)NCCOc1nccnc1Nc1ccc(C(F)(F)F)cc1]

To a stirred mixture of 3-(2-aminoethoxy)-N-[4-(trifluoromethyl)phenyl]pyrazin-2-amine (240 mg, 0.80 mmol, 1 equiv) and TEA (244.27 mg, 2.41 mmol, 3 equiv) in DCM (5 ml) was added prop-2-enoyl chloride (76.47 mg, 0.84 mmol, 1.05 equiv) dropwise at room temperature. The resulting mixture was stirred for 1 h at room temperature. Desired product could be detected by LCMS. The resulting mixture was diluted with water (30 mL). The resulting mixture was extracted with CH₂Cl₂ (3×30 mL). The combined organic layers were washed with water (2×30 mL), dried over anhydrous Na₂SO₄. After filtration, the filtrate was concentrated under reduced pressure. The crude product was purified by reverse phase flash with the following conditions (Acetonitrile: Water/0.05% ammonia water 25%-80% 19 min) to afford N-{2-[(3-{[4-(trifluoromethyl)phenyl]amino}pyrazin-2-yl)oxy]ethyl}prop-2-enamide (30 mg) as a white solid. LCMS:(ES, m/z): [M+H]⁺=353. ¹H NMR (400 MHz, DMSO-d₆) (8.84 (s, 1H), 8.42 (s, 1H), 8.11 (d, J=8.8 Hz, 2H), 7.78 (d, J=3.0 Hz, 1H), 7.68 (d, J=8.6 Hz, 2H), 7.59 (d, J=3.0 Hz, 1H), 6.26 (d, J=9.8 Hz, 1H), 6.15 (d, J=17.2 Hz, 1H), 5.63 (d, J=10.0 Hz, 1H), 4.42 (t, J=5.5 Hz, 2H), 3.65 (d, J=5.6 Hz, 2H).

### Example 193. Synthesis of N-(prop-2-yn-1-yl)-N-(2-((3-((4-(trifluoromethyl)phenyl)amino)pyrazin-2-yl)oxy)ethyl)acrylamide

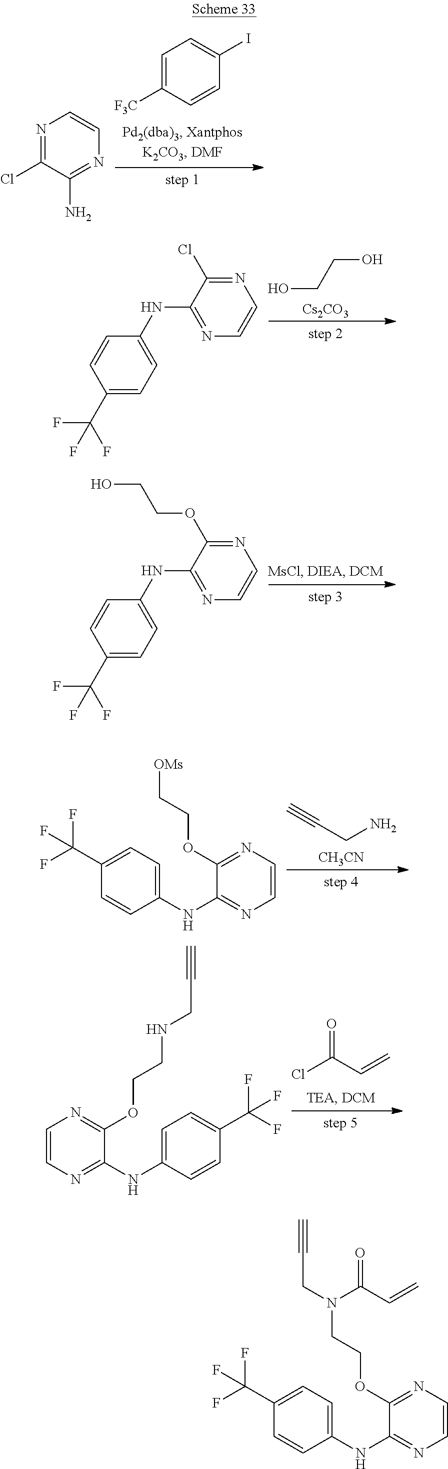

### Step 1: Synthesis of 3-chloro-N-[4-(trifluoromethyl)phenyl]pyrazin-2-amine

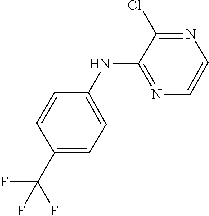 [SMILES: FC(F)(F)c1ccc(Nc2nccnc2Cl)cc1]

A solution of 3-chloropyrazin-2-amine (1.50 g, 11.60 mmol, 1.00 equiv), 1-iodo-4-(trifluoromethyl) (3.47 g, 12.75 mmol,1.10 equiv) and Cs₂CO₃ (11.31 g, 34.8 mmol, 3.00 equiv), Xantphos (1.34 g, 2.2 mmol, 0.20 equiv) and Pd₂(dba)₃ (1.05 g, 1.16 mmol, 0.10 equiv) in DMF (20.00 mL) was stirred for 2 h at 90° C. under N₂ atmosphere. The reaction was allowed to cool down to room temperature and extracted with ethyl acetate (3×30.00 ml). The combined organic layers were washed with brine, dried over anhydrous Na₂SO₄. After filtration, the filtrate was concentrated under reduced pressure. The residue was purified by silica gel column and eluted with ethyl acetate: petroleum ether=5:1 to afford 3-chloro-N-[4-(trifluoromethyl)phenyl]pyrazin-2-amine (2.04 g, 64.39%) as a yellow solid.

### Step 2: Synthesis of 2-[(3-{[4-(trifluoromethyl)phenyl]amino}pyrazin-2-yl)oxy]ethanol

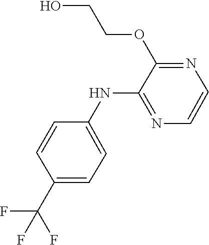 [SMILES: OCCOc1nccnc1Nc1ccc(C(F)(F)F)cc1]

To a solution of 3-chloro-N-[4-(trifluoromethyl)phenyl]pyrazin-2-amine (1.50 g, 5.48 mmol, 1.00 equiv) in ethylene glycol (30.00 mL) were added Cs₂CO₃ (5.37 g, 16.44 mmol, 3.00 equiv) at room temperature. The resulting mixture was stirred for additional 1 h at 100° C. The reaction was quenched by the addition of H₂O (100.00 ml) at room temperature. The resulting mixture was extracted with EA (3×80.00 ml). The combined organic layers were washed with brine (150.00 ml), dried over anhydrous Na₂SO₄. After filtration, the filtrate was concentrated under reduced pressure to afford 2-[(3-{[4-(trifluoromethyl)phenyl]amino}pyrazin-2-yl)oxy]ethanol (1.60 g, 97.54%) as organic-yellow solid. LCMS:(ES, m/z): [M−H]—=298.1.

### Step 3: Synthesis of 2-[(3-{[4-(trifluoromethyl)phenyl]amino}pyrazin-2-yl)oxy]ethyl methanesulfonate

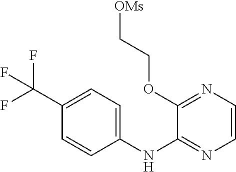 [SMILES: CS(=O)(=O)OCCOc1nccnc1Nc1ccc(C(F)(F)F)cc1]

A solution of 2-[(3-{[4-(trifluoromethyl)phenyl]amino}pyrazin-2-yl)oxy]ethanol (1.00 g, 3.34 mmol, 1.00 equiv) and DIEA (1.30 g, 10.02 mmol, 3.00 equiv) in DCM (20.00 mL, 3.34 mmol). To the above mixture was dropwise methanesulfonyl chloride (0.46 g, 4.01 mmol, 1.20 equiv) at ice bath. The resulting mixture was stirred for additional 1 h at 0° C.-room temperature. The reaction was quenched with H₂O (50.00 mL) at room temperature. The resulting mixture was extracted with DCM (3×20.00 ml). The combined organic layers were washed with brine (50.00 ml), dried over anhydrous Na₂SO₄. After filtration, the filtrate was concentrated under reduced pressure to afford 2-[(3-{[4-(trifluoromethyl)phenyl]amino}pyrazin-2-yl)oxy]ethyl methanesulfonate (0.80 g, 63.44%) as a yellow oil. LCMS:(ES, m/z): [M−H]—=376.1.

### Step 4: Synthesis of 3-[2-(prop-2-yn-1-ylamino)ethoxy]-N-[4-(trifluoromethyl)phenyl]pyrazin-2-amine

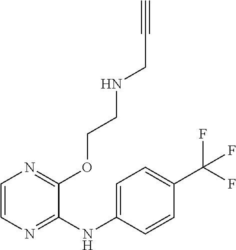 [SMILES: C#CCNCCOc1nccnc1Nc1ccc(C(F)(F)F)cc1]

A solution of 2-[(3-{[4-(trifluoromethyl)phenyl]amino}pyrazin-2-yl)oxy]ethyl methanesulfonate (800.00 mg, 2.12 mmol, 1.00 equiv) and 2-propynylamine (583.88 mg, 10.60 mmol, 5.00 equiv) in acetonitrile (20.00 mL) was stirred for 4 h at 80° C. The reaction was quenched with H₂O (60.00 ml) at ice bath. The resulting mixture was extracted with EA (3×25.00 ml). The combined organic layers were washed with brine (80.00 ml), dried over anhydrous Na₂SO₄. After filtration, the filtrate was concentrated under reduced pressure to afford 3-[2-(prop-2-yn-1-ylamino)ethoxy]-N-[4-(trifluoromethyl)phenyl]pyrazin-2-amine (600.00 mg, 84.15%) as a yellow oil. LCMS:(ES, m/z): [M+H]⁺=337.2.

### Step 5: Synthesis ofN-(prop-2-yn-1-yl)-N-{2-[(3-{[4-(trifluoromethyl)phenyl]amino}pyrazin-2-yl)oxy]ethyl}prop-2-enamide

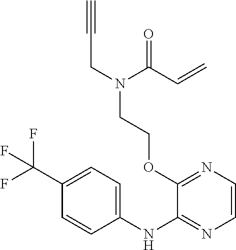 [SMILES: C#CCN(CCOc1nccnc1Nc1ccc(C(F)(F)F)cc1)C(=O)C=C]

To a solution of 3-[2-(prop-2-yn-1-ylamino) ethoxy]-N-[4-(trifluoromethyl)phenyl]pyrazin-2-amine (600.00 mg, 1.78 mmol, 1.00 equiv) and TEA (541.59 mg, 5.35 mmol, 3.00 equiv) in DCM (10.00 mL). was added acryloyl chloride (161.47 mg, 1.78 mmol, 1.00 equiv) dropwise at ice bath. The resulting mixture was stirred for additional 1 h at 0° C.-room temperature. The reaction was quenched by the addition of H₂O (50.00 ml) at ice bath. The resulting mixture was extracted with DCM (3×50.00 ml). The combined organic layers were washed with brine (100.00 ml), dried over anhydrous Na₂SO₄. After filtration, the filtrate was concentrated under reduced pressure. The residue was purified by silica gel column chromatography, eluted with PE/THF (1:2), The crude product was purified by (Column: Welch Xtimate C18 ExRS, 250 mm, 10Im;Mobile Phase A:Water (0.05% NH₃·H₂O), Mobile Phase B:ACN; Flow rate:45 mL/min;Gradient:366% B to 80% B in 10 min,80% B; Wave Length: 254 nm; RT1(min): 7; Number Of Runs:5) with the following conditions to afford N-(prop-2-yn-1-yl)-N-{2-[(3-{[4-(trifluoromethyl)phenyl]amino}pyrazin-2-yl)oxy]ethyl}prop-2-enamide (80 mg, 11.49%) as a white solid. LCMS:(ES, m/z): [M+H]⁺=391.2. ¹HNMR (400 MHz, DMSO-d₆): δ 9.60-9.54 (m, 1H), 8.21 (d, J=8.4 Hz, 2H), 7.64 (d, J=8.0 Hz, 2H), 7.12-6.90 (m, 2H), 6.76-6.62 (m, 1H), 6.11 (t, J=12.0 Hz, 1H). 5.72-5.70 (m, 1H), 4.33-4.29 (m, 2H), 4.12 (br, s, 2H), 3.86-3.79 (m, 2H), 3.21 (s, 1H).

### Example 194. Synthesis of N-((4-(6-(trifluoromethyl)pyridin-3-yl)-1,2,3,4-tetrahydropyrazino[2,3-b]pyrazin-2-yl)methyl)acrylamide

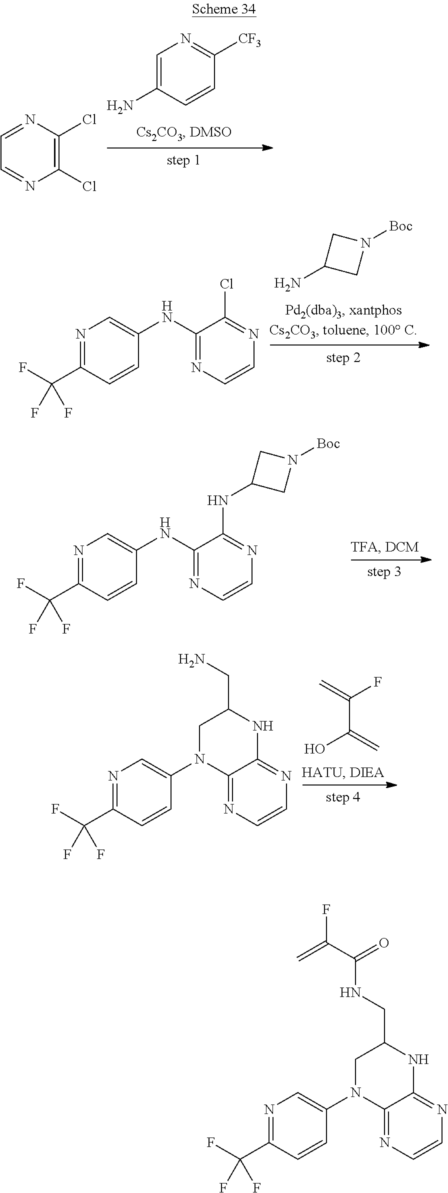

### Step 1: Synthesis of 3-chloro-N-(6-(trifluoromethyl)pyridin-3-yl)pyrazin-2-amine

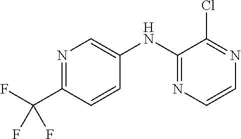 [SMILES: FC(F)(F)c1ccc(Nc2nccnc2Cl)cn1]

To a stirred solution of 2,3-dichloropyrazine (45 g, 302.074 mmol, 1 equiv) and 6-(trifluoromethyl) pyridin-3-amine (58.76 g, 362.489 mmol, 1.2 equiv) in DMSO (500 mL) were added Cs₂CO₃ (196.84 g, 604.148 mmol, 2 equiv) at room temperature. This resulting mixture was stirred for 12 hours at 100° C. To the resulting mixture was added H₂O (500 mL) and extracted with EA (3×500 mL). The combined organic layers were washed with H₂O (3×500 mL), dried over anhydrous Na₂SO₄. After filtration, the filtrate was concentrated under reduced pressure and purified by column chromatography on silica gel (Combiflash, THF/PE: 15%) to give 3-chloro-N-[6-(trifluoromethyl)pyridin-3-yl]pyrazin-2-amine (40 g, 48.22%) as a yellow solid.

### Step 2: Synthesis of tert-butyl 3-[(3-{1[6-(trifluoromethyl) pyridin-3-yl]amino}pyrazine-2-yl)amino]azetidine-1-carboxylate

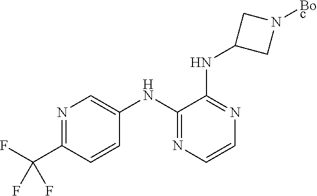 [SMILES: CC(C)(C)OC(=O)N1CC(Nc2nccnc2Nc2ccc(C(F)(F)F)nc2)C1]

To a stirred solution of 3-chloro-N-[6-(trifluoromethyl) pyridin-3-yl]pyrazin-2-amine (40 g, 145.651 mmol, 1 equiv) and tert-butyl 3-aminoazetidine-1-carboxylate (30.10 g, 174.781 mmol, 1.2 equiv) in toluene (400 mL) were added Cs₂CO₃ (94.91 g, 291.302 mmol, 2 equiv), Xantphos (16.86 g, 29.130 mmol, 0.2 equiv) and Pd₂(dba)₃ (13.34 g, 14.565 mmol, 0.1 equiv) at room temperature. This resulting mixture was stirred for 2 hours at 100° C. under N₂ atmosphere. The mixture was concentrated under reduced pressure and purified by column chromatography on silica gel (THF/PE: 30%) to give tert-butyl 3-[(3-{[6-(trifluoromethyl) pyridin-3-yl]amino}pyrazine-2-yl)amino]azetidine-1-carboxylate (43 g, 71.94%) as a yellow solid. LCMS (ES, m/z): [M+H]⁺=411.

### Step 3: Synthesis of (4-(6-(trifluoromethyl)pyridin-3-yl)-1,2,3,4-tetrahydropyrazino[2,3-b]pyrazin-2-yl)methanamine

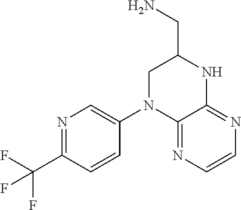 [SMILES: NCC1CN(c2ccc(C(F)(F)F)nc2)c2nccnc2N1]

To a solution of tert-butyl 3-[(3-{[6-(trifluoromethyl) pyridin-3-yl]amino}pyrazine-2-yl)amino]azetidine-1-carboxylate (43 g, 104.878 mmol, 1 equiv) in DCM (200 mL) was added TFA (100 mL) dropwised at 0° C. The resulting mixture was stirred for 1 h at room temperature, then concentrated under reduced pressure. To the crude was added water (200 mL), adjusted the pH to 8-9 with saturated NaHCO₃ aqueous, then extracted with EA (3×300 mL). The combined organic layers were dried over anhydrous Na₂SO₄ and concentrated to give (4-(6-(trifluoromethyl) pyridin-3-yl)-1,2,3,4-tetrahydropyrazino[2,3-b]pyrazin-2-yl) methanamine (30 g, 96.77%). The crude product was used in the next step directly without further purification. LCMS (ES, m/z): [M+H]⁺=311

### Step 4: Synthesis of N-((4-(6-(trifluoromethyl)pyridin-3-yl)-1,2,3,4-tetrahydropyrazino[2,3-b]pyrazin-2-yl)methyl)acrylamide

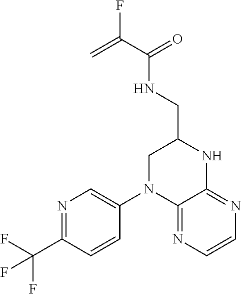 [SMILES: C=C(F)C(=O)NCC1CN(c2ccc(C(F)(F)F)nc2)c2nccnc2N1]

To a stirred solution of 1-{4-[6-(trifluoromethyl) pyridin-3-yl]-1H,2H,3H-[1,4]diazino[2,3-b]pyrazin-2-yl}methanamine (2 g, 6.446 mmol, 1 equiv), 2-fluoroprop-2-enoic acid (0.70 g, 7.735 mmol, 1.2 equiv) and DIEA (4.17 g, 32.230 mmol, 5 equiv) in DCM (20 mL) was added HATU (3.67 g, 9.669 mmol, 1.5 equiv) at 0° C., then stirred at room temperature for 2 hours. To the mixture was added water (50 mL) and extracted with DCM (3×50 mL). The combined organic phase was washed with brine (1×100 mL), dried over anhydrous Na₂SO₄ and concentrated under reduced pressure. The residue was purified by column chromatography on silica gel (Combiflash) (PE/THF=1:1) to give the crude, which was triturated with ether (20 mL) and resulted in 2-fluoro-N-({4-[6-(trifluoromethyl) pyridin-3-yl]-1H,2H,3H-[1,4]diazino[2,3-b]pyrazin-2-yl}methyl) prop-2-enamide (500 mg, 20.29%) as a white solid. LCMS-PH-CAL-Y-V2458766-0: (ES, m/z): [M+H]⁺=383. ¹H NMR (400 MHz, DMSO-d₆) δ 9.44 (s, 1H), 9.20 (d, J=2.5 Hz, 1H), 8.73-8.61 (m, 2H), 7.80 (d, J=8.7 Hz, 1H), 6.98 (d, J=4.6 Hz, 1H), 6.53 (d, J=4.5 Hz, 1H), 5.55 (dd, J=48.1, 3.5 Hz, 1H), 5.28 (dd, J=15.7, 3.4 Hz, 1H), 4.45-4.32 (m, 1H), 4.14 (t, J=11.3 Hz, 1H), 3.91 (dd, J=11.4, 7.8 Hz, 1H), 3.52-3.42 (m, 1H), 3.29-3.18 (m, 1H).

### BIOLOGICAL EXAMPLES

### General Procedures

### NCI-H226 Proliferation Assay

NCI-H226 cells were obtained from American Type Culture Collection (ATCC, CRL-5826) and cultured in RPMI 1640 with L-glutamine (Corning, 10-040-CV) supplemented with 10% fetal bovine serum (Gibco, 10438-026) and 1% penicillin-streptomycin (Gibco, 15070063). Cells were trypsinized, resuspended in growth medium, and diluted to a concentrated of 10,000 cells/mL. 500 cells, 50 μL of cell suspension, per well were then plated in a white 384-well plate (Corning) and incubated at 37° C., 5% CO₂. After 24 hours incubation, 100 nL of test compounds in DMSO, 20-point 1:2 dilution starting from 20 μM (final concentration) in triplicate, were transferred to each well using an Agilent Bravo instrument outfitted with a pintool head. Cells were incubated at 37° C., 5% CO₂ for 5 days followed by the addition of 30 μL of Cell TiterGlo (Promega; diluted 1:6 in water) to each well and luminescence values recorded using an EnVision plate reader.

NCI-H4226 Proliferation Assay (From Wuxi AppTec CO.):

Day −1: Seeding cells
(1) Equilibrate cell culture medium to 37° C.
(2) Remove and discard the culture medium.
(3) Briefly rinse the cell layer with PBS to remove all traces of serum that contains trypsin.
(4) Add 2-3 mL of Trypsin-EDTA solution to flask and observe cells under an inverted microscope until cell layer is dispersed.
(5) Add 6-8 mL of growth medium to terminate the digestion of Trypsin/EDTA.
(6) Centrifuge the mixture (Trypsin EDTA and the growth medium) at 1000 rpm for 5 mins.
(7) Discard the supernatant, suspend the cell precipitation with the fresh growth medium, then take 1 mL the cell suspension to count cells with Vi-cell XR cell viability analyzer.
(8) Adjust the cell density to 500 cells/well for 384-well plates.
(9) Add 50 L/well volume cell suspension to the assay plate.
(10) Add 50 L media to the control columns.
(11) Incubate the plate at 37° C., 5% CO₂ for overnight.
   Day 0: Compounds treatment
(1) Add cpds to each well by Tecan HP D300E. The top dose is 10 VM, 3-fold dilution and 9 doses. DMSO final concentration will be normalized to 0.2%.
(2) Incubate the assay plate at 37° C., 5% CO₂ for 7 days.
   Day 7: Plate read
(1) Equilibrate the assay plate to the room temperature for nearly 30 minutes.
(2) Add 25 L CellTiter-Glo reagent to each well and shake the plate for 1 min, incubate 10 min and keep them from light.
(3) Detect the plate by EnVision.
   Data analysis:

$$Data is analyzed using XL fit , Inhibition \% = ( 1 - ( Sample value - Min ) / ( Max - Min ) ) * 100.$$

Max: Average of the DMSO group
Min: Average of medium group.

### TEAD Reporter Assay:

MCF7 TEAD-LUC reporter cells were obtained from BPS Bioscience, Inc. and cultured in DMEM medium (Corning, 10-013-CV) supplemented with 10% fetal bovine serum (Gibco, 10438-026), 1% penicillin-streptomycin (Gibco, 15070063), 1% non-essential amino acids (Gibco, 15070063), and 10 μg/mL insulin (Sigma, 10516). Cells were trypsinized, resuspended in growth medium, and diluted to a concentrated of 100,000 cells/mL. 5,000 cells, 50 μL of cell suspension, per well were then plated in a white 384-well plate (Corning) and incubated at 37° C., 5% CO₂. After 24 hours incubation, 100 nL of test compounds in DMSO, 20-point 1:2 dilution starting from 20 μM (final concentration) in triplicate, were transferred to each well using an Agilent Bravo instrument outfitted with a pintool head. Cells were incubated at 37° C., 5% CO₂ for 24 hours followed by the addition of 30 μL of BrightGlo (Promega; diluted 1:3 in water) to each well and luminescence values recorded using an EnVision plate reader.

### IMR32 Cytoxicity Assay:

IMR32 cells were obtained from American Type Culture Collection (ATCC, CCL-127) and cultured in DMEM (Corning, 10-013-CV) supplemented with 10% fetal bovine serum (Gibco, 10438-026) and 1% penicillin-streptomycin (Gibco, 15070063). Cells were trypsinized, resuspended in growth medium, and diluted to a concentrated of 20,000 cells/mL. 1,000 cells, 50 μL of cell suspension, per well were then plated in a white 384-well plate (Corning) and incubated at 37° C., 5% CO₂. After 24 hours incubation, 100 nL of test compounds in DMSO, 20-point 1:2 dilution starting from 20 μM (final concentration) in triplicate, were transferred to each well using an Agilent Bravo instrument outfitted with a pintool head. Cells were incubated at 37° C., 5% CO₂ for 3 days followed by the addition of 30 μL of Cell TiterGlo (Promega; diluted 1:6 in water) to each well and luminescence values recorded using an EnVision plate reader.

The in vitro assay results of the compounds in the present invention were included in Table 1. The compounds described herein, inhibited the transcriptional activity of TEADs, as assessed by decreased luminance from a TEAD reporter cell line (MCF7 TEAD-LUC) in response to compound treatment. The compounds also inhibited growth of a malignant mesothelioma cell line, NCI-H226, which relies on YAP/TEAD activity for growth.¹³ In contrast, the compounds typically did not inhibit the growth of IMR32 neuroblastoma cells at relevant concentrations (>1 μM). Because neuroblastomas typically do not express YAP and do not depend on TEADs for growth, these data indicate that the compounds specifically inhibit growth by targeting TEADs.¹⁴

Pharmacokinetic Studies in mice: The plasma pharmacokinetic profiles of representative compounds were evaluated in mouse via intravenous (2 mg/kg in 75% PEG 300+25% D5W solution) and oral (10 mg/kg in 75% PEG 300+25% D5W solution) dosing routes. These data (Table 2) indicated that the compounds have high oral bioavailability in mice.

TABLE 2
Pharmacokinetic parameters for representative examples

| Compound | 1 | 39 | 45 | 79 |
|---|---|---|---|---|
| Cl (ml/min/kg) | 42.2 | 39.5 | 16.5 | 47.3 |
| Vd (L/kg) | 7.0 | 1.8 | 0.54 | 1.73 |
| T_{1/2} (h) | 2.1 | 0.5 | 0.4 | 0.4 |
| F % | 94.4 | 90 | 54.0 | 109 |

Numbered references in the present disclosure are as follows:
[1] Moya, I. M., and Halder, G. (2019) Hippo-YAP/TAZ signalling in organ regeneration and regenerative medicine, Nat Rev Mol Cell Biol 20, 211-226.
[2] Ma, S., Meng, Z., Chen, R., and Guan, K. L. (2019) The Hippo Pathway: Biology and Pathophysiology, Annu Rev Biochem 88, 577-604.
[3] Huh, H. D., Kim, D. H., Jeong, H. S., and Park, H. W. (2019) Regulation of TEAD Transcription Factors in Cancer Biology, Cells 8.
[4] Calses, P. C., Crawford, J. J., Lill, J. R., and Dey, A. (2019) Hippo Pathway in Cancer: Aberrant Regulation and Therapeutic Opportunities, Trends Cancer 5, 297-307.
[5] Yang, H., Hall, S. R. R., Sun, B., Zhao, L., Gao, Y., Schmid, R. A., Tan, S. T., Peng, R. W., and Yao, F. (2021) NF2 and Canonical Hippo-YAP Pathway Define Distinct Tumor Subsets Characterized by Different Immune Deficiency and Treatment Implications in Human Pleural Mesothelioma, Cancers (Basel) 13.
[6] Feng, J., Yang, H., Zhang, Y., Wei, H., Zhu, Z., Zhu, B., Yang, M., Cao, W., Wang, L., and Wu, Z. (2017) Tumor cell-derived lactate induces TAZ-dependent upregulation of PD-L1 through GPR81 in human lung cancer cells, Oncogene 36, 5829-5839.
[7] Guo, X., Zhao, Y., Yan, H., Yang, Y., Shen, S., Dai, X., Ji, X., Ji, F., Gong, X. G., Li, L., Bai, X., Feng, X. H., Liang, T., Ji, J., Chen, L., Wang, H., and Zhao, B. (2017) Single tumor-initiating cells evade immune clearance by recruiting type II macrophages, Genes Dev 31, 247-259.
[8] Wang, G., Lu, X., Dey, P., Deng, P., Wu, C. C., Jiang, S., Fang, Z., Zhao, K., Konaparthi, R., Hua, S., Zhang, J., Li-Ning-Tapia, E. M., Kapoor, A., Wu, C. J., Patel, N. B., Guo, Z., Ramamoorthy, V., Tieu, T. N., Heffernan, T., Zhao, D., Shang, X., Khadka, S., Hou, P., Hu, B., Jin, E. J., Yao, W., Pan, X., Ding, Z., Shi, Y., Li, L., Chang, Q., Troncoso, P., Logothetis, C. J., McArthur, M. J., Chin, L., Wang, Y. A., and DePinho, R. A. (2016) Targeting YAP-Dependent MDSC Infiltration Impairs Tumor Progression, Cancer Discov 6, 80-95.
[9] Ni, X., Tao, J., Barbi, J., Chen, Q., Park, B. V., Li, Z., Zhang, N., Lebid, A., Ramaswamy, A., Wei, P., Zheng, Y., Zhang, X., Wu, X., Vignali, P., Yang, C. P., Li, H., Pardoll, D., Lu, L., Pan, D., and Pan, F. (2018) YAP Is Essential for Treg-Mediated Suppression of Antitumor Immunity, Cancer Discov 8, 1026-1043.
[10] Stampouloglou, E., Cheng, N., Federico, A., Slaby, E., Monti, S., Szeto, G. L., and Varelas, X. (2020) Yap suppresses T-cell function and infiltration in the tumor microenvironment, PLoS Biol 18, e3000591.
[11] Currey, L., Thor, S., and Piper, M. (2021) TEAD family transcription factors in development and disease, Development 148.
[12] Chan, P., Han, X., Zheng, B., DeRan, M., Yu, J., Jarugumilli, G. K., Deng, H., Pan, D., Luo, X., and Wu, X. (2016) Autopalmitoylation of TEAD proteins regulates transcriptional output of the Hippo pathway, Nat Chem Biol 12, 282-289.
[13] Tang, T. T., Konradi, A. W., Feng, Y., Peng, X., Ma, M., Li, J., Yu, F. X., Guan, K. L., and Post, L. (2021) Small Molecule Inhibitors of TEAD Auto-palmitoylation Selectively Inhibit Proliferation and Tumor Growth of NF2-deficient Mesothelioma, Mol Cancer Ther 20, 986-998.
[14] Pearson, J. D., Huang, K., Pacal, M., McCurdy, S. R., Lu, S., Aubry, A., Yu, T., Wadosky, K. M., Zhang, L., Wang, T., Gregorieff, A., Ahmad, M., Dimaras, H., Langille, E., Cole, S. P. C., Monnier, P. P., Lok, B. H., Tsao, M. S., Akeno, N., Schramek, D., Wikenheiser-Brokamp, K. A., Knudsen, E. S., Witkiewicz, A. K., Wrana, J. L., Goodrich, D. W., and Bremner, R. (2021) Binary pan-cancer classes with distinct vulnerabilities defined by pro- or anti-cancer YAP/TEAD activity, Cancer Cell 39, 1115-1134 e1112.
[15] Kurppa, K. J., Liu, Y., To, C., Zhang, T., Fan, M., Vajdi, A., Knelson, E. H., Xie, Y., Lim, K., Cejas, P., Portell, A., Lizotte, P. H., Ficarro, S. B., Li, S., Chen, T., Haikala, H. M., Wang, H., Bahcall, M., Gao, Y., Shalhout, S., Boettcher, S., Shin, B. H., Thai, T., Wilkens, M. K., Tillgren, M. L., Mushajiang, M., Xu, M., Choi, J., Bertram, A. A., Ebert, B. L., Beroukhim, R., Bandopadhayay, P., Awad, M. M., Gokhale, P. C., Kirschmeier, P. T., Marto, J. A., Camargo, F. D., Haq, R., Paweletz, C. P., Wong, K. K., Barbie, D. A., Long, H. W., Gray, N. S., and Janne, P. A. (2020) Treatment-Induced Tumor Dormancy through YAP-Mediated Transcriptional Reprogramming of the Apoptotic Pathway, Cancer Cell 37, 104-122 e112.

The foregoing disclosure has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the disclosure should, therefore, be determined not with reference to the above description, but should instead be determined with reference to the following appended claims, along with the full scope of equivalents to which such claims are entitled.

This application refers to various issued patents, published patent applications, journal articles, and other publications, each of which are incorporated herein by reference.

We claim:

1. A compound of Formula (I), or a pharmaceutically acceptable salt thereof: 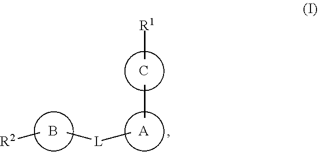
wherein
Ring A is a 6-membered aryl or 6-membered heteroaryl having 1, 2, or 3 nitrogen ring members,
Ring B is a C₆-C₁₂-aryl, C₄-C₇-cycloalkyl, or 6-membered heteroaryl having 1 or 2 nitrogen ring members;
Ring C is a 4- to 10-membered heterocycle containing 1 or 2 N ring members and that is optionally fused and optionally has 1 to 3 unsaturated bonds;
L is selected from the group consisting of —NH—, —O—, —CH₂—, —S—, —C(O)—, —CHOH—, and a bond;
wherein
when R¹ is bound to a carbon ring member, then
R¹ is selected from the group consisting of CN, halo, —[CH₂]_{0,1}NHC(O)(C₁-C₆-alkyl), —[CH₂]_{0,1}NHC(O)(C₂-C₆-alkenyl), and —[CH₂]_{0,1}NHC(O)(C₂-C₆-alkynyl), —[CH₂]_{0,1}NHC(O)(C₃-C₁₀-cycloalkyl), —[CH₂]_{0,1}NHC(O)(3- to 6-membered heterocycloalkyl (wherein 1-4 ring members are independently selected from N, O, and S)), —[CH₂]_{0,1}NHSO₂(C₁-C₆-alkyl), —[CH₂]_{0,1}NHSO₂(C₂-C₆-alkenyl), and —[CH₂]_{0,1}NHSO₂(C₂-C₆-alkynyl), —[CH₂]_{0,1}NHSO₂(C₃-C₁₀-cycloalkyl), and —[CH₂]_{0,1}NHSO₂(3- to 6-membered heterocycloalkyl (wherein 1-4 ring members are independently selected from N, O, and S))
when R¹ is bound to a nitrogen ring member, then
R¹ is selected from the group consisting of CN, —C(O)(C₁-C₆-alkyl), —C(O)(C₂-C₆-alkenyl), —C(O)(C₂-C₆-alkynyl), —C(O)(C₃-C₁₀-cycloalkyl), —C(O)(3- to 6-membered heterocycloalkyl (wherein 1-4 ring members are independently selected from N, O, and S)), —C₁-C₆-alkyl(C₃-C₁₀-cycloalkyl), —SO₂(C₁-C₆-alkyl), —SO₂(C₂-C₆-alkenyl), —SO₂(C₂-C₆-alkynyl), —SO₂(C₃-C₁₀-cycloalkyl), and —SO₂(3- to 6-membered heterocycloalkyl (wherein 1-4 ring members are independently selected from N, O, and S));
R² is selected from the group consisting of halo, C₁-C₆-haloalkyl, C₁-C₆-haloalkoxy, and —SF₅,
Ring A, Ring B, and Ring C are independently and optionally substituted with 1 to 3 three substituents independently selected from halo and C₁-C₆-alkyl; and
any alkyl, alkenyl, alkynyl, cycloalkyl, and heterocycloalkyl is optionally substituted with one to three substituents independently selected from the group consisting of halo, OH, CN, C₁-C₆-alkoxy, C₁-C₆-alkyl, C₁-C₆-haloalkyl, C₁-C₆-hydroxyalkyl, (C₀-C₆-alkyl)NRR′, —(C₀-C₆-alkyl)-OC(O)R, —(C₀-C₆-alkyl)-C(O)OR, C₃-C₁₀-cycloalkyl, 3- to 6-membered heterocycloalkyl (wherein 1-4 ring members are independently selected from N, O, and S), —(C₁-C₆-alkyl)(3- to 6-membered heterocycloalkyl (wherein 1-4 ring members are independently selected from N, O, and S),
wherein R and R′ are independently selected from H, C₁-C₆-alkyl, C₂-C₆-alkenyl, and C₂-C₆-alkynyl).

2. The compound or pharmaceutically acceptable salt thereof according to claim 1, wherein Ring A is 6-membered heteroaryl having 2 or 3 nitrogen ring members

3. The compound or pharmaceutically acceptable salt thereof according to claim 1 or 2, wherein Ring A is selected from the group consisting of optionally substituted:

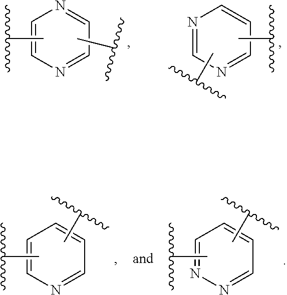

4. The compound or pharmaceutically acceptable salt thereof according to any one of claims 1 to 3, wherein Ring A is optionally substituted:

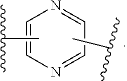

5. The compound or pharmaceutically acceptable salt thereof according to any one of claims 1 to 3, wherein Ring A is optionally substituted:

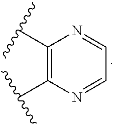 [SMILES: *c1nccnc1*]

6. The compound or pharmaceutically acceptable salt thereof according to any one of claims 1 to 5, wherein Ring B is optionally substituted C₆-C₁₂-aryl.

7. The compound or pharmaceutically acceptable salt thereof according to any one of claims 1 to 6, wherein Ring B is optionally substituted phenyl.

8. The compound or pharmaceutically acceptable salt thereof according to any one of claims 1 to 5, wherein Ring B is optionally substituted 6-membered heteroaryl having 1 or 2 nitrogen ring members.

9. The compound or pharmaceutically acceptable salt thereof according to any one of claims 1 to 5 and 8, wherein Ring B is selected from the group of optionally substituted:

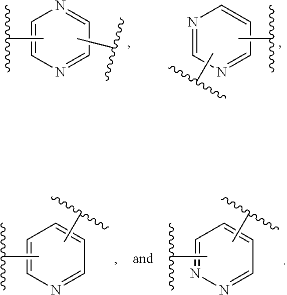

10. The compound or pharmaceutically acceptable salt thereof according to any one of claims 1 to 9, wherein Ring C is an optionally substituted 4- to 8-membered heterocycle.

11. The compound or pharmaceutically acceptable salt thereof according to any one of claims 1 to 10, wherein Ring C contains 1 N ring member.

12. The compound or pharmaceutically acceptable salt thereof according to any one of claims 1 to 10, wherein Ring C contains 2 N ring members.

13. The compound or pharmaceutically acceptable salt thereof according to any one of claims 1 to 10, wherein Ring C is selected from the group consisting of optionally substituted:

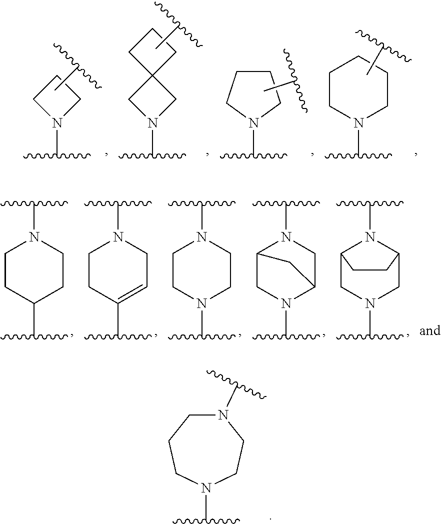

14. The compound or pharmaceutically acceptable salt thereof according to any one of claims 1 to 11, wherein Ring C is optionally substituted:

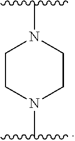 [SMILES: *N1CCN(*)CC1]

15. The compound or pharmaceutically acceptable salt thereof according to any one of claims 1 to 14, wherein L is —NH—.

16. The compound or pharmaceutically acceptable salt thereof according to any one of claims 1 to 15, wherein R² is C₁-C₆-haloalkyl.

17. The compound or pharmaceutically acceptable salt thereof according to any one of claims 1 to 16, wherein R² is —CF₃.

18. The compound or pharmaceutically acceptable salt thereof according to any one of claims 1 to 17, wherein R¹ is bound to a nitrogen ring member.

19. The compound or pharmaceutically acceptable salt thereof according to claim 18, wherein R¹ has the formula: 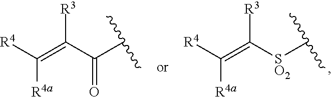
wherein R³, R⁴, and R^{4a} are individually selected from the group consisting of H, halo, CN, —C(O)OR, and optionally substituted C₁-C₆-alkyl.

20. The compound or pharmaceutically acceptable salt thereof according to claim 19, wherein R¹ has the formula:

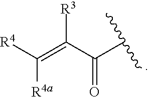

21. The compound or pharmaceutically acceptable salt thereof according to claim 19 or 20, wherein:
R³ is selected from the group consisting of H, F, C₁, CH₂F, CF₃, CN, CH₂OH, CH₂OC(O)R, and CH₂NRR′; and
R⁴ and R^{4a} are independently selected from the group consisting of H, CH₂F, CHF₂, CH₂OH, CH₂NRR′, and CH₂-(3- to 6-membered heterocycloalkyl (wherein 1-4 ring members are independently selected from N, O, and S).

22. The compound or pharmaceutically acceptable salt thereof according to claim 18, wherein R¹ has the formula: 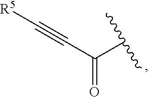
wherein R⁵ is selected from the group consisting of H, C₁-C₆-alkyl, C₁-C₆-haloalkyl, and C₁-C₆-hydroxyalkyl.

23. The compound or pharmaceutically acceptable salt thereof according to claim 18, wherein R¹ has the formula: 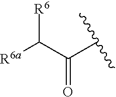
wherein R⁶ and R^{6a} are independently selected from the group consisting of H, halo, CN, and C₁-C₆-alkyl.

24. The compound or pharmaceutically acceptable salt thereof according to claim 23, wherein at least one of R⁶ and R^{6a} is halo.

25. The compound or pharmaceutically acceptable salt thereof according to claim 23 or 24, wherein each of R⁶ and R^{6a} is halo.

26. The compound or pharmaceutically acceptable salt thereof according to claim 23 or 24, wherein at least one of R⁶ and R^{6a} is CN.

27. The compound or pharmaceutically acceptable salt thereof according to claim 18, wherein R′has the formula: 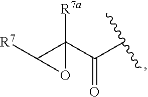
wherein R⁷ and R^{7a} are independently selected from the group consisting of H, optionally substituted alkyl, and —C(O)OR.

28. The compound or pharmaceutically acceptable salt thereof according to claim 1 or 18, wherein R′is selected from the group consisting of: 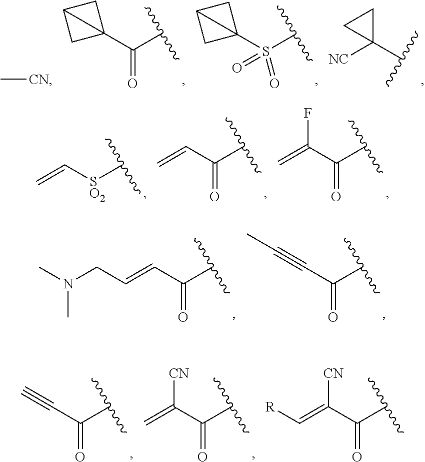
(wherein R is substituted alkyl),

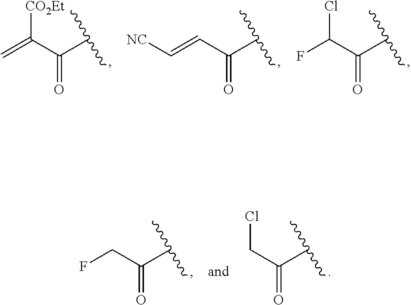

29. The compound or pharmaceutically acceptable salt thereof according to claim 1, 18, or 28, wherein R¹ is:

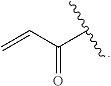 [SMILES: *C(=O)C=C]

30. The compound or pharmaceutically acceptable salt thereof according to claim 1, wherein the compound is of Formula (IA):

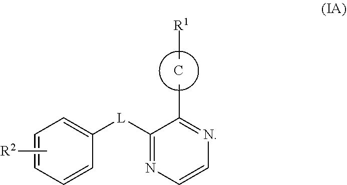

31. The compound or pharmaceutically acceptable salt thereof according to claim 30, wherein L is —NH—.

32. The compound or pharmaceutically acceptable salt thereof according to claim 30 or 31, wherein R² is —CF₃ or —SFS.

33. The compound or pharmaceutically acceptable salt thereof according to any one of claims 30 to 32, wherein R² is in the para position.

34. The compound or pharmaceutically acceptable salt thereof according to any one of claims 30 to 33, wherein Ring C is a 4- to 6-membered heterocycle containing 2 N ring members.

35. The compound or pharmaceutically acceptable salt thereof according to any one of claims 30 to 34, wherein Ring C is:

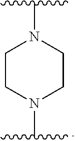 [SMILES: *N1CCN(*)CC1]

36. The compound or pharmaceutically acceptable salt thereof according to claim 1, wherein the compound is selected from the following:
1-(4-(3-((4-(trifluoromethyl)phenyl)amino)pyrazin-2-yl)piperazin-1-yl)prop-2-en-1-one;
4-(3-((4-(trifluoromethyl)phenyl)amino)pyrazin-2-yl)piperazine-1-carbonitrile;
N-(1-(3-((4-(trifluoromethyl)phenyl)amino)pyrazin-2-yl)azetidin-3-yl)acrylamide;
N-((1r,3r)-3-((3-((4-(trifluoromethyl)phenyl)amino)pyrazin-2-yl)amino)cyclobutyl) acrylamide;
N-((1s,3s)-3-((3-((4-(trifluoromethyl)phenyl)amino)pyrazin-2-yl)amino)cyclobutyl) acrylamide;
N-(2-(3-((4-(trifluoromethyl)phenyl)amino)pyrazin-2-yl)-2-azaspiro[3.3]heptan-6-yl)acrylamide;
N-(1-(3-((4-(trifluoromethyl)phenyl)amino)pyrazin-2-yl)pyrrolidin-3-yl)acrylamide;
1-(3-((3-((4-(trifluoromethyl)phenyl)amino)pyrazin-2-yl)amino)pyrrolidin-1-yl)prop-2-en-1-one;
N-(1-(3-((4-(trifluoromethyl)phenyl)amino)pyrazin-2-yl)piperidin-4-yl)acrylamide;
N-((1-(3-((4-(trifluoromethyl)phenyl)amino)pyrazin-2-yl)piperidin-3-yl)methyl)acrylamide;
1-(5-(3-((4-(trifluoromethyl)phenyl)amino)pyrazin-2-yl)-2,5-diazabicyclo[2.2.1]heptan-2-yl)prop-2-en-1-one;
1-((1R,5S)-3-(3-((4-(trifluoromethyl)phenyl)amino)pyrazin-2-yl)-3,8-diazabicyclo[3.2.1]octan-8-yl)prop-2-en-1-one;
(R)-1-(2-methyl-4-(3-((4-(trifluoromethyl)phenyl)amino)pyrazin-2-yl)piperazin-1-yl)prop-2-en-1-one;
(S)-1-(2-methyl-4-(3-((4-(trifluoromethyl)phenyl)amino)pyrazin-2-yl)piperazin-1-yl)prop-2-en-1-one;
(R)-1-(2-(hydroxymethyl)-4-(3-((4-(trifluoromethyl)phenyl)amino)pyrazin-2-yl)piperazin-1-yl)prop-2-en-1-one;
(S)-1-(2-(hydroxymethyl)-4-(3-((4-(trifluoromethyl)phenyl)amino)pyrazin-2-yl)piperazin-1-yl)prop-2-en-1-one;
(S)-1-(2-(2-hydroxyethyl)-4-(3-((4-(trifluoromethyl)phenyl)amino)pyrazin-2-yl)piperazin-1-yl)prop-2-en-1-one;
(R)-1-acryloyl-4-(3-((4-(trifluoromethyl)phenyl)amino)pyrazin-2-yl)piperazine-2-carboxamide;
(S)-1-acryloyl-4-(3-((4-(trifluoromethyl)phenyl)amino)pyrazin-2-yl)piperazine-2-carboxamide;
2-(1-acryloyl-4-(3-((4-(trifluoromethyl)phenyl)amino)pyrazin-2-yl)piperazin-2-yl)acetamide;
(R)-2-(1-acryloyl-4-(3-((4-(trifluoromethyl)phenyl)amino)pyrazin-2-yl)piperazin-2-yl)acetonitrile;
(S)-2-(1-acryloyl-4-(3-((4-(trifluoromethyl)phenyl)amino)pyrazin-2-yl)piperazin-2-yl)acetonitrile;
1-(2-(prop-2-yn-1-yl)-4-(3-((4-(trifluoromethyl)phenyl)amino)pyrazin-2-yl)piperazin-1-yl)prop-2-en-1-one;
1-(3-methyl-4-(3-((4-(trifluoromethyl)phenyl)amino)pyrazin-2-yl)piperazin-1-yl)prop-2-en-1-one;
1-[4-(3-{[4-(trifluoromethyl)phenyl]amino}pyrazin-2-yl)-3, 6-dihydro-2H-pyridin-1-yl]prop-2-en-1-one;
1-[4-(3-{[4-(trifluoromethyl)phenyl]amino}pyrazin-2-yl)piperidin-1-yl]prop-2-en-1-one;
1-(4-(3-((4-(trifluoromethyl)phenyl)amino)pyrazin-2-yl)-1,4-diazepan-1-yl)prop-2-en-1-one;
1-(4-(3-(4-(trifluoromethyl)phenyl)pyrazin-2-yl)piperazin-1-yl)prop-2-en-1-one;
1-(4-(3-(3-(trifluoromethyl)phenyl)pyrazin-2-yl)piperazin-1-yl)prop-2-en-1-one;
1-(4-(3-((3-(trifluoromethyl)phenyl)amino)pyrazin-2-yl)piperazin-1-yl)prop-2-en-1-one;
1-(4-(3-((4-(pentafluoro-16-sulfaneyl)phenyl)amino)pyrazin-2-yl)piperazin-1-yl)prop-2-en-1-one;
1-(4-(3-((4-chlorophenyl)amino)pyrazin-2-yl)piperazin-1-yl)prop-2-en-1-one;
1-(4-(3-((4-fluorophenyl)amino)pyrazin-2-yl)piperazin-1-yl)prop-2-en-1-one;
1-(4-(3-((4-(trifluoromethoxy)phenyl)amino)pyrazin-2-yl)piperazin-1-yl)prop-2-en-1-one;
1-(4-(3-((4-(difluoromethoxy)phenyl)amino)pyrazin-2-yl)piperazin-1-yl)prop-2-en-1-one;
1-(4-(3-((4-(fluoromethoxy)phenyl)amino)pyrazin-2-yl)piperazin-1-yl)prop-2-en-1-one;
1-(4-(3-((3-methoxy-4-(trifluoromethyl)phenyl)amino)pyrazin-2-yl)piperazin-1-yl)prop-2-en-1-one;
4-((3-(4-acryloylpiperazin-1-yl)pyrazin-2-yl)amino)benzonitrile;
1-(4-(3-((5-(trifluoromethyl)pyridin-2-yl)amino)pyrazin-2-yl)piperazin-1-yl)prop-2-en-1-one;
1-(4-(3-((5-(difluoromethyl)pyridin-2-yl)amino)pyrazin-2-yl)piperazin-1-yl)prop-2-en-1-one;
1-(4-(3-((5-(trifluoromethyl)pyrimidin-2-yl)amino)pyrazin-2-yl)piperazin-1-yl)prop-2-en-1-one;
1-(4-(3-(methyl(5-(trifluoromethyl)pyridin-2-yl)amino)pyrazin-2-yl)piperazin-1-yl)prop-2-en-1-one;
1-(4-(3-((5-(trifluoromethyl)pyridin-2-yl)amino)pyrazin-2-yl)-3, 6-dihydropyridin-1(2H)-yl)prop-2-en-1-one;
1-(4-(3-((5-(trifluoromethyl)pyridin-2-yl)amino)pyrazin-2-yl)piperidin-1-yl)prop-2-en-1-one;
1-(4-(3-((5-(trifluoromethyl)pyridin-2-yl)amino)pyrazin-2-yl)piperazin-1-yl)prop-2-en-1-one;
2-fluoro-1-(4-(3-((6-(trifluoromethyl)pyridin-3-yl)amino)pyrazin-2-yl)piperazin-1-yl)prop-2-en-1-one;
1-(4-(3-((6-(difluoromethyl)pyridin-3-yl)amino)pyrazin-2-yl)piperazin-1-yl)prop-2-en-1-one;
1-(4-(3-((6-(trifluoromethoxy)pyridin-3-yl)amino)pyrazin-2-yl)piperazin-1-yl)prop-2-en-1-one;
1-(4-(3-((6-(difluoromethoxy)pyridin-3-yl)amino)pyrazin-2-yl)piperazin-1-yl)prop-2-en-1-one;
1-(4-(3-((5-fluoro-6-(trifluoromethyl)pyridin-3-yl)amino)pyrazin-2-yl)piperazin-1-yl)prop-2-en-1-one;
2-fluoro-1-(4-(3-((5-fluoro-6-(trifluoromethyl)pyridin-3-yl)amino)pyrazin-2-yl)piperazin-1-yl)prop-2-en-1-one;
1-(4-(3-((2-(trifluoromethyl)pyrimidin-5-yl)amino)pyrazin-2-yl)piperazin-1-yl)prop-2-en-1-one;
(S)-1-(2-methyl-4-(3-((6-(trifluoromethyl)pyridin-3-yl)amino)pyrazin-2-yl)piperazin-1-yl)prop-2-en-1-one;
(R)-1-(2-methyl-4-(3-((6-(trifluoromethyl)pyridin-3-yl)amino)pyrazin-2-yl)piperazin-1-yl)prop-2-en-1-one;
(R)-1-(2-methyl-4-(3-((6-(trifluoromethyl)pyridin-3-yl)amino)pyrazin-2-yl)piperazin-1-yl)prop-2-en-1-one;
(R)-1-(2-methyl-4-(3-((6-(trifluoromethyl)pyridin-3-yl)amino)pyrazin-2-yl)piperazin-1-yl)prop-2-en-1-one;
(R)-1-(2-methyl-4-(3-((6-(trifluoromethyl)pyridin-3-yl)amino)pyrazin-2-yl)piperazin-1-yl)prop-2-en-1-one;
2-fluoro-1-(4-(3-((6-(trifluoromethoxy)pyridin-3-yl)amino)pyrazin-2-yl)-3,6-dihydropyridin-1(2H)-yl)prop-2-en-1-one;
2-fluoro-1-(4-(3-((6-(trifluoromethoxy)pyridin-3-yl)amino)pyrazin-2-yl)-3,6-dihydropyridin-1(2H)-yl)prop-2-en-1-one;
2-fluoro-1-(4-(3-((2-(trifluoromethyl)pyrimidin-5-yl)amino)pyrazin-2-yl)-3,6-dihydropyridin-1(2H)-yl)prop-2-en-1-one;
1-(4-(3-((6-(trifluoromethyl)pyridin-3-yl)amino)pyrazin-2-yl)piperidin-1-yl)prop-2-en-1-one;
1-(3-((3-((6-(trifluoromethyl)pyridin-3-yl)amino)pyrazin-2-yl)amino)azetidin-1-yl)prop-2-en-1-one;
2-fluoro-1-(3-((3-((6-(trifluoromethyl)pyridin-3-yl)amino)pyrazin-2-yl)amino)azetidin-1-yl)prop-2-en-1-one;
2-fluoro-1-(3-((3-((6-(trifluoromethoxy)pyridin-3-yl)amino)pyrazin-2-yl)amino)azetidin-1-yl)prop-2-en-1-one;
2-fluoro-1-(3-((3-((6-(trifluoromethyl)pyridin-3-yl)amino)pyrazin-2-yl)oxy)azetidin-1-yl)prop-2-en-1-one;
1-(4-(3-((4,4-difluorocyclohexyl)amino)pyrazin-2-yl)piperazin-1-yl)prop-2-en-1-one;
1-(4-(3-((4,4-difluorocyclohexyl)amino)pyrazin-2-yl)piperazin-1-yl)prop-2-en-1-one;
2-fluoro-1-(4-(3-((4-(trifluoromethyl)bicyclo[1.1.1]pentan-2-yl)amino)pyrazin-2-yl)piperazin-1-yl)prop-2-en-1-one;
1-{4-[3-(4-tert-butylphenoxy)pyrazin-2-yl]piperazin-1-yl}prop-2-en-1-one;
1-(4-(3-(4-methoxyphenoxy)pyrazin-2-yl)piperazin-1-yl)prop-2-en-1-one;
1-(4-(3-(4-(difluoromethoxy)phenoxy)pyrazin-2-yl)piperazin-1-yl)prop-2-en-1-one;
1-(4-(3-(4-(trifluoromethoxy)phenoxy)pyrazin-2-yl)piperazin-1-yl)prop-2-en-1-one;
1-(4-(3-((5-(trifluoromethyl)pyridin-2-yl)oxy)pyrazin-2-yl)piperazin-1-yl)prop-2-en-1-one;
1-(4-(3-((5-(trifluoromethyl)pyridin-2-yl)oxy)pyrazin-2-yl)-3, 6-dihydropyridin-1(2H)-yl)prop-2-en-1-one;
1-(4-(3-((5-(trifluoromethyl)pyridin-2-yl)oxy)pyrazin-2-yl)-3, 6-dihydropyridin-1(2H)-yl)prop-2-en-1-one;
2-fluoro-1-(4-(3-((5-(trifluoromethyl)pyridin-2-yl)oxy)pyrazin-2-yl)piperidin-1-yl)prop-2-en-1-one;
1-(4-(3-((6-(trifluoromethyl)pyridin-3-yl)oxy)pyrazin-2-yl)piperazin-1-yl)prop-2-en-1-one;
2-fluoro-1-(4-(3-((6-(trifluoromethyl)pyridin-3-yl)oxy)pyrazin-2-yl)piperazin-1-yl)prop-2-en-1-one;
1-(4-(3-((6-(trifluoromethyl)pyridin-3-yl)oxy)pyrazin-2-yl)piperidin-1-yl)prop-2-en-1-one;
2-fluoro-1-(4-(3-((6-(trifluoromethyl)pyridin-3-yl)oxy)pyrazin-2-yl)piperidin-1-yl)prop-2-en-1-one;
2-(trifluoromethyl)-1-(4-(3-((6-(trifluoromethyl)pyridin-3-yl)oxy)pyrazin-2-yl)piperidin-1-yl)prop-2-en-1-one;
2-((4-(3-((6-(trifluoromethyl)pyridin-3-yl)oxy)pyrazin-2-yl)piperidin-1-yl)methyl)acrylic acid;
1-(4-hydroxy-4-(3-((6-(trifluoromethyl)pyridin-3-yl)oxy)pyrazin-2-yl)piperidin-1-yl)prop-2-en-1-one;
1-(4-(3-((6-(trifluoromethyl)pyridin-3-yl)oxy)pyrazin-2-yl)-3, 6-dihydropyridin-1(2H)-yl)prop-2-en-1-one;
2-fluoro-1-(4-(3-((6-(trifluoromethyl)pyridin-3-yl)oxy)pyrazin-2-yl)-3,6-dihydropyridin-1(2H)-yl)prop-2-en-1-one;
2-((3-(3-((6-(trifluoromethyl)pyridin-3-yl)oxy)pyrazin-2-yl)azetidin-1-yl)methyl)acrylic acid;
1-(4-(3-((2-(trifluoromethyl)pyrimidin-5-yl)oxy)pyrazin-2-yl)piperazin-1-yl)prop-2-en-1-one;
2-fluoro-1-(4-(3-((2-(trifluoromethyl)pyrimidin-5-yl)oxy)pyrazin-2-yl)piperazin-1-yl)prop-2-en-1-one;
1-(4-(3-((2-(trifluoromethyl)pyrimidin-5-yl)oxy)pyrazin-2-yl)-3, 6-dihydropyridin-1(2H)-yl)prop-2-en-1-one;
2-fluoro-1-(4-(3-((2-(trifluoromethyl)pyrimidin-5-yl)oxy)pyrazin-2-yl)-3,6-dihydropyridin-1(2H)-yl)prop-2-en-1-one;
1-(4-(3-((2-(trifluoromethyl)pyrimidin-5-yl)oxy)pyrazin-2-yl)piperidin-1-yl)prop-2-en-1-one;
2-fluoro-1-(4-(3-((2-(trifluoromethyl)pyrimidin-5-yl)oxy)pyrazin-²-yl)piperidin-1-yl)prop-2-en-1-one;
1-(4-{3-[(4,4-difluorocyclohexyl)oxy]pyrazin-2-yl}piperazin-1-yl) prop-2-en-1-one;
1-(4-(3-((4-(trifluoromethyl)cyclohexyl)oxy)pyrazin-2-yl)piperazin-1-yl)prop-2-en-1-one;
1-(4-(3-(4-(trifluoromethyl)benzoyl)pyrazin-2-yl)piperazin-1-yl)prop-2-en-1-one;
1-(4-(3-(6-(trifluoromethyl)nicotinoyl)pyrazin-2-yl)piperazin-1-yl)prop-2-en-1-one;
1-(4-(3-(hydroxy(4-(trifluoromethyl)phenyl)methyl)pyrazin-2-yl)piperazin-1-yl)prop-2-en-1-one;
1-(4-(3-(4-(trifluoromethyl)benzyl)pyrazin-2-yl)piperazin-1-yl)prop-2-en-1-one;
3-(4-acryloylpiperazin-1-yl)-N-cyclobutylpyrazine-2-carboxamide;
3-(4-acryloylpiperazin-1-yl)-N-(3,3-difluorocyclobutyl)pyrazine-2-carboxamide;
1-(4-(3-(6-(trifluoromethyl)-2-azaspiro[3.3]heptane-2-carbonyl)pyrazin-2-yl)piperazin-1-yl)prop-2-en-1-one;
3-(4-acryloylpiperazin-1-yl)-N-cyclopentylpyrazine-2-carboxamide;
1-(4-(3-(4-(trifluoromethyl)piperidine-1-carbonyl)pyrazin-2-yl)piperazin-1-yl)prop-2-en-1-one;
1-(4-(3-(4,4-difluoropiperidine-1-carbonyl)pyrazin-2-yl)piperazin-1-yl)prop-2-en-1-one;
1-(4-(3-(4-(trifluoromethyl)piperidin-1-yl)pyrazin-2-yl)piperazin-1-yl)prop-2-en-1-one;
1-(4-(3-(3-phenylazetidin-1-yl)pyrazin-2-yl)piperazin-1-yl)prop-2-en-1-one;
1-(4-(3-(3-(4-fluorophenyl)azetidin-1-yl)pyrazin-2-yl)piperazin-1-yl)prop-2-en-1-one;
1-(3-((3-(3-(4-fluorophenyl)azetidin-1-yl)pyrazin-2-yl)amino)azetidin-1-yl)prop-2-en-1-one;
2-fluoro-1-(3-((3-(3-(4-fluorophenyl)azetidin-1-yl)pyrazin-2-yl)amino)azetidin-1-yl)prop-2-en-1-one;
1-(4-(3-(6-(trifluoromethyl)-2-azaspiro[3.3]heptan-2-yl)pyrazin-2-yl)piperazin-1-yl)prop-2-en-1-one;
1-(4-(5-hydroxy-3-((4-(trifluoromethyl)phenyl)amino)pyrazin-2-yl)piperazin-1-yl)prop-2-en-1-one;
1-(4-(6-methyl-3-((4-(trifluoromethyl)phenyl)amino)pyrazin-2-yl)piperazin-1-yl)prop-2-en-1-one;
1-(4-(5,6-dimethyl-3-((6-(trifluoromethyl)pyridin-3-yl)amino)pyrazin-2-yl)piperazin-1-yl)prop-2-en-1-one;
1-(4-(5,6-dimethyl-3-((6-(trifluoromethyl)pyridin-3-yl)amino)pyrazin-2-yl)piperazin-1-yl)-²-fluoroprop-2-en-1-one;
2-fluoro-1-(4-(6-methyl-3-((6-(trifluoromethyl)pyridin-3-yl)oxy)pyrazin-2-yl)piperidin-1-yl)prop-2-en-1-one;
1-(4-(5-methyl-3-((6-(trifluoromethyl)pyridin-3-yl)oxy)pyrazin-2-yl)piperidin-1-yl)prop-2-en-1-one;
2-fluoro-1-(4-(5-methyl-3-((6-(trifluoromethyl)pyridin-3-yl)oxy)pyrazin-2-yl)piperidin-1-yl)prop-2-en-1-one;
3-(1-acryloylpiperidin-4-yl)-1-(5-(trifluoromethyl)pyrimidin-2-yl)pyrazin-2(1H)-one;
1-(4-(3-((4-(trifluoromethyl)phenyl)amino)pyridazin-4-yl)piperazin-1-yl)prop-2-en-1-one;
1-(4-(3-((5-(trifluoromethyl)pyridin-2-yl)amino)pyridazin-4-yl)piperazin-1-yl)prop-2-en-1-one;
1-(4-(3-((6-(trifluoromethyl)pyridin-3-yl)amino)pyridazin-4-yl)piperazin-1-yl)prop-2-en-1-one;
1-(4-(5-((4-(trifluoromethyl)phenyl)amino)pyridazin-4-yl)piperazin-1-yl)prop-2-en-1-one;
1-[4-(2-{[4-(trifluoromethyl)phenyl]amino}pyrimidin-4-yl)piperazin-1-yl]prop-2-en-1-one;
1-(4-(methyl(2-((4-(trifluoromethyl)phenyl)amino)pyrimidin-4-yl)amino)piperidin-1-yl)prop-2-en-1-one;
1-[4-(4-{[4-(trifluoromethyl)phenyl]amino}pyrimidin-2-yl)piperazin-1-yl]prop-2-en-1-one;
1-(4-(4-((6-(trifluoromethyl)pyridin-3-yl)oxy)pyrimidin-5-yl)piperidin-1-yl)prop-2-en-1-one;
2-fluoro-1-(4-(4-((6-(trifluoromethyl)pyridin-3-yl)oxy)pyrimidin-5-yl)piperidin-1-yl)prop-2-en-1-one;
1-(4-(2-methyl-5-((4-(trifluoromethyl)phenyl)amino)pyrimidin-4-yl)piperazin-1-yl)prop-2-en-1-one;
1-(4-(2-methyl-4-((6-(trifluoromethyl)pyridin-3-yl)oxy)pyrimidin-5-yl)-3,6-dihydropyridin-1(2H)-yl)prop-2-en-1-one;
2-fluoro-1-(4-(2-methyl-4-((6-(trifluoromethyl)pyridin-3-yl)oxy)pyrimidin-5-yl)-3,6-dihydropyridin-1(2H)-yl)prop-2-en-1-one;
1-(4-(2-methyl-4-((6-(trifluoromethyl)pyridin-3-yl)oxy)pyrimidin-5-yl)piperidin-1-yl)prop-2-en-1-one;
2-fluoro-1-(4-(2-methyl-4-((6-(trifluoromethyl)pyridin-3-yl)oxy)pyrimidin-5-yl)piperidin-1-yl)prop-2-en-1-one;
1-(4-(2-methoxy-5-((4-(trifluoromethyl)phenyl)amino)pyrimidin-4-yl)piperazin-1-yl)prop-2-en-1-one;
1-[4-(3-{[4-(trifluoromethyl)phenyl]amino}pyridin-2-yl)piperazin-1-yl]prop-2-en-1-one;
2-fluoro-1-(4-(3-((6-(trifluoromethyl)pyridin-3-yl)amino)pyridin-2-yl)piperazin-1-yl)prop-2-en-1-one;
1-(3-((6-(trifluoromethyl)pyridin-3-yl)oxy)-3′,6′-dihydro-[2,4′-bipyridin]-1′(2′H)-yl)prop-2-en-1-one;
1-(3-((6-(trifluoromethyl)pyridin-3-yl)oxy)-3′,6′-dihydro-[2,4′-bipyridin]-1′(2′H)-yl)prop-2-en-1-one;
1-(4-(3-((6-(trifluoromethyl)pyridin-3-yl)oxy)pyridin-2-yl)piperidin-1-yl)prop-2-en-1-one
2-fluoro-1-(4-(3-((6-(trifluoromethyl)pyridin-3-yl)oxy)pyridin-2-yl)piperidin-1-yl)prop-2-en-1-one;
1-(4-(3-(5-(trifluoromethyl)picolinoyl)pyridin-2-yl)piperazin-1-yl)prop-2-en-1-one;
1-(4-(3-(6-(trifluoromethyl)nicotinoyl)pyridin-2-yl)piperazin-1-yl)prop-2-en-1-one;
1-(4-(3-(hydroxy(6-(trifluoromethyl)pyridin-3-yl)methyl)pyridin-2-yl)piperazin-1-yl)prop-2-en-1-one;
1-[4-(2-{[4-(trifluoromethyl)phenyl]amino}pyridin-3-yl)piperazin-1-yl]prop-2-en-1-one;
of 2-fluoro-1-(4-(2-((6-(trifluoromethyl)pyridin-3-yl)amino)pyridin-3-yl)piperazin-1-yl)prop-2-en-1-one;
1-(2-((6-(trifluoromethyl)pyridin-3-yl)amino)-3′,6′-dihydro-[3,4′-bipyridin]-1′(2′H)-yl)prop-2-en-1-one;
2-fluoro-1-(2-((6-(trifluoromethyl)pyridin-3-yl)amino)-3′,6′-dihydro-[3,4′-bipyridin]-1′(2′H)-yl)prop-2-en-1-one;
1-(2-((6-(trifluoromethoxy)pyridin-3-yl)amino)-3′,6′-dihydro-[3,4′-bipyridin]-1′(2′H)-yl)prop-2-en-1-one;
2-fluoro-1-(2-((6-(trifluoromethoxy)pyridin-3-yl)amino)-3′,6′-dihydro-[3,4′-bipyridin]-1′(2′H)-yl)prop-2-en-1-one;
1-(4-(2-((6-(trifluoromethyl)pyridin-3-yl)amino)pyridin-3-yl)piperidin-1-yl)prop-2-en-1-one;
2-fluoro-1-(4-(2-((6-(trifluoromethyl)pyridin-3-yl)amino)pyridin-3-yl)piperidin-1-yl)prop-2-en-1-one;
1-(4-(2-((6-(trifluoromethoxy)pyridin-3-yl)amino)pyridin-3-yl)piperidin-1-yl)prop-2-en-1-one;
2-fluoro-1-(4-(2-((6-(trifluoromethoxy)pyridin-3-yl)amino)pyridin-3-yl)piperidin-1-yl)prop-2-en-1-one;
1-(2-((6-(trifluoromethyl)pyridin-3-yl)oxy)-3′,6′-dihydro-[3,4′-bipyridin]-1′(2′H)-yl)prop-2-en-1-one;
2-fluoro-1-(2-((6-(trifluoromethyl)pyridin-3-yl)oxy)-3′,6′-dihydro-[3,4′-bipyridin]-1′(2′H)-yl)prop-2-en-1-one;
1-(4-(2-((6-(trifluoromethyl)pyridin-3-yl)oxy)pyridin-3-yl)piperidin-1-yl)prop-2-en-1-one;
2-fluoro-1-(4-(2-((6-(trifluoromethyl)pyridin-3-yl)oxy)pyridin-3-yl)piperidin-1-yl)prop-2-en-1-one;
1-(2-((2-(trifluoromethyl)pyrimidin-5-yl)oxy)-3′,6′-dihydro-[3,4′-bipyridin]-1′(2′H)-yl)prop-2-en-1-one;
2-fluoro-1-(2-((2-(trifluoromethyl)pyrimidin-5-yl)oxy)-3′,6′-dihydro-[3,4′-bipyridin]-1′(2′H)-yl)prop-2-en-1-one;
1-(4-(2-((2-(trifluoromethyl)pyrimidin-5-yl)oxy)pyridin-3-yl)piperidin-1-yl)prop-2-en-1-one;
2-fluoro-1-(4-(2-((2-(trifluoromethyl)pyrimidin-5-yl)oxy)pyridin-3-yl)piperidin-1-yl)prop-2-en-1-one;
1-(4-(3-(4,4-difluoropiperidine-1-carbonyl)pyridin-2-yl)piperazin-1-yl)prop-2-en-1-one;
1-(4-(2-((4-(trifluoromethyl)phenyl)amino)phenyl)piperazin-1-yl)prop-2-en-1-one;
1-(4-(3-(4-(trifluoromethyl)benzoyl)-1H-pyrazol-1-yl)piperidin-1-yl)prop-2-en-1-one;
1-(4-(5-(4-(trifluoromethyl)benzoyl)-1H-pyrazol-1-yl)piperidin-1-yl)prop-2-en-1-one;
1-(4-(5-methyl-3-(4-(trifluoromethyl)benzoyl)-1H-pyrazol-1-yl)piperidin-1-yl)prop-2-en-1-one;
1-(4-(3-methyl-5-(4-(trifluoromethyl)benzoyl)-1H-pyrazol-1-yl)piperidin-1-yl)prop-2-en-1-one;
4-(dimethylamino)-1-(4-(3-((4-(trifluoromethyl)phenyl)amino)pyrazin-2-yl)piperazin-1-yl)but-2-en-1-one;
1-(4-(3-((4-(trifluoromethyl)phenyl)amino)pyrazin-2-yl)piperazin-1-yl)ethan-1-one;
2-chloro-2-fluoro-1-(4-(3-((4-(trifluoromethyl)phenyl)amino)pyrazin-2-yl)piperazin-1-yl)ethan-1-one;
2-chloro-1-(4-(3-((4-(trifluoromethyl)phenyl)amino)pyrazin-2-yl)piperazin-1-yl)ethan-1-one;
3-oxo-3-(4-(3-((4-(trifluoromethyl)phenyl)amino)pyrazin-2-yl)piperazin-1-yl)propanenitrile;
oxiran-2-yl(4-(3-((4-(trifluoromethyl)phenyl)amino)pyrazin-2-yl)piperazin-1-yl)methanone;
1-(4-(3-((4-(trifluoromethyl)phenyl)amino)pyrazin-2-yl)piperazin-1-yl)prop-2-yn-1-one;
1-(4-(3-((4-(trifluoromethyl)phenyl)amino)pyrazin-2-yl)piperazin-1-yl)but-2-yn-1-one;
2-(4-(3-((4-(trifluoromethyl)phenyl)amino)pyrazin-2-yl)piperazin-1-yl)acetonitrile;
2-methyl-2-(4-(3-((4-(trifluoromethyl)phenyl)amino)pyrazin-2-yl)piperazin-1-yl)propanenitrile;
2-methyl-1-(4-(3-((6-(trifluoromethyl)pyridin-3-yl)amino)pyrazin-2-yl)piperazin-1-yl)prop-2-en-1-one;
(E)-1-(4-(3-((6-(trifluoromethyl)pyridin-3-yl)amino)pyrazin-2-yl)piperazin-1-yl)but-2-en-1-one;
(E)-4-(methyl(prop-2-yn-1-yl)amino)-1-(4-(3-((4-(trifluoromethyl)phenyl)amino)pyrazin-2-yl)piperazin-1-yl)but-2-en-1-one;
2,3-dihydroxy-1-(4-(3-((4-(trifluoromethyl)phenyl)amino)pyrazin-2-yl)piperazin-1-yl)propan-1-one;
(E)-4-oxo-4-(4-(3-((4-(trifluoromethyl)phenyl)amino)pyrazin-2-yl)piperazin-1-yl)but-2-enenitrile;
(Z)-4-oxo-4-(4-(3-((4-(trifluoromethyl)phenyl)amino)pyrazin-2-yl)piperazin-1-yl)but-2-enenitrile;
(E)-4-oxo-4-(4-(3-((6-(trifluoromethyl)pyridin-3-yl)amino)pyrazin-2-yl)piperazin-1-yl)but-2-enenitrile;
(Z)-4-oxo-4-(4-(3-((6-(trifluoromethyl)pyridin-3-yl)amino)pyrazin-2-yl)piperazin-1-yl)but-2-enenitrile;
N-cyano-N-methyl-4-(3-((4-(trifluoromethyl)phenyl)amino)pyrazin-2-yl)piperazine-1-carboxamide;
N-cyano-N-methyl-4-(3-((4-(trifluoromethyl)phenyl)amino)pyrazin-2-yl)piperazine-1-carboxamide;
2-(4-(3-((4-(trifluoromethyl)phenyl)amino)pyrazin-2-yl)piperazine-1-carbonyl)allyl acetate;
2-(fluoromethyl)-1-(4-(3-((4-(trifluoromethyl)phenyl)amino)pyrazin-2-yl)piperazin-1-yl)prop-2-en-1-one;
2-(pyrrolidin-1-ylmethyl)-1-(4-(3-((4-(trifluoromethyl)phenyl)amino)pyrazin-2-yl)piperazin-1-yl)prop-2-en-1-one;
2-(trifluoromethoxy)-1-(4-(3-((4-(trifluoromethyl)phenyl)amino)pyrazin-2-yl)piperazin-1-yl)ethan-1-one;
N-methyl-N-(2-((3-((4-(trifluoromethyl)phenyl)amino)pyrazin-2-yl)oxy)ethyl)acrylamide;
N-(2-((3-((4-(trifluoromethyl)phenyl)amino)pyrazin-2-yl)oxy)ethyl)acrylamide;
N-(prop-2-yn-1-yl)-N-(2-((3-((4-(trifluoromethyl)phenyl)amino)pyrazin-2-yl)oxy)ethyl)acrylamide; and
N-((4-(6-(trifluoromethyl)pyridin-3-yl)-1,2,3,4-tetrahydropyrazino[2,3-b]pyrazin-2-yl)methyl)acrylamide.

37. A pharmaceutical composition comprising a compound or pharmaceutically acceptable salt thereof according to any one of claims 1 to 36 and a pharmaceutically acceptable carrier.

38. A method for treating a disease in a subject suffering therefrom, comprising administering to the subject a compound or pharmaceutically acceptable salt thereof according to any one of claims 1 to 36, wherein the disease is selected from the group consisting of Diabetic foot ulcer (DFU), Venous Ulcer (Stasis Ulcer), Pressure Ulcers, Full or partial thickness burns, Eczema, Psoriasis, Cellulitis, Impetigo, Atopic dermatitis, Epidermolysis Bullosa, Lichen Sclerosis, Ichthyosis, Vitiligo, Acral peeling skin syndrome, Blau syndrome, Primary cutaneous amyloidosis, Cutaneous abscess, Blepharitis, Furunculosis, Capillaritis, Cellulitis, Corneal Abrasion, Corneal Erosion, Xerosis, Lichen Planus, Lichen Simplex Chronicus, Idiopathic pulmonary fibrosis (IPF), Acute respiratory distress syndrome (ARDS), Chronic Obstructive Pulmonary Disease (COPD), Emphysema, Silicosis, Asbestosis, Pneumoconiosis, Aluminosis, Bauxite fibrosis, Berylliosis, Siderosis, Stannosis, Pulmonary Talcosis, Labrador lung (mixed dust Pneumoconiosis), Sarcoidosis, Hypersensitivity pneumonitis (HP)/extrinsic allergic alveolitis (EAA), Desquamative interstitial pneumonia (DIP), Respiratory bronchiolitis interstitial lung disease (RBILD), Acute interstitial pneumonia (AIP), Nonspecific interstitial pneumonia (NSIP), Cryptogenic organizing pneumonia (COP=idiopathic BOOP), Secondary organizing pneumonia (BOOP), Lymphoid interstitial pneumonia (LIP), Idiopathic interstitial pneumonia: unspecified, Hypereosinophilic lung diseases, Tuberculosis (TB), Pulmonary Edema, Interstitial Lung Disease, Cryptogenic Organizing Pneumonia (COP), E-cigarette or Vaping Use-Associated Lung Injury (EVALI), Hantavirus Pulmonary Syndrome (HPS), Histoplasmosis, Legionnaires' Disease, MAC Lung Disease, Alpha-1 Antitrypsin Deficiency, Aspergillosis, Lymphangioleiomyomatosis (LAM), Middle Eastern Respiratory Syndrome (MERS), Nontuberculous Mycobacterial Lung Disease (NTM), Pulmonary Embolism Goodpasture syndrome, idiopathic pulmonary hemosiderosis, Alveolar proteinosis, Pulmonary amyloidosis, Primary pulmonary lymphoma, Primary ciliary dyskinesia (without or with situs inversus), Rare cause of hypersensitivity pneumonitis (all causes other than farmer's lung disease and pigeon breeder's lung disease), Pulmonary arteriovenous malformations in hereditary hemorrhagic telangiectasia (HHT), interstitial lung disease in systemic sclerosis, interstitial lung disease in rheumatoid arthritis, interstitial lung disease in idiopathic inflammatory myopathies (polymyositis, dermatomyositis, anti-synthetase syndrome), interstitial lung disease in Sjögren syndrome, interstitial lung disease in mixed connective tissue disease (MCTD), interstitial lung disease in overlap syndromes, interstitial lung disease in undifferentiated connective tissue disease, Bronchiolitis obliterans (in non-transplanted patients), Infectious colitis, Ulcerative colitis, Crohn's disease, Ischemic colitis, Radiation colitis, Peptic ulcer, Intestinal cancer, Intestinal obstruction, Rheumatoid arthritis, Psoriatic arthritis, Hashimoto thyroiditis, Systemic lupus erythematosus, Multiple Sclerosis, Graves' Disease, Type 1 Diabetes Mellitus, Psoriasis, Ankylosing spondylitis, Scleroderma, Myositis, Gout, Antiphospholipid Antibody Syndrome (APS), Vasculitis, Dilated cardiomyopathy, Hypertrophic cardiomyopathy, Restrictive cardiomyopathy, Systolic heart failure, Diastolic heart failure (heart failure with preserved ejection fraction), Atrial Septal Defect, Atrioventricular Septal Defect, Coarctation of the Aorta, Double-outlet Right Ventricle, d-Transposition of the Great Arteries, Ebstein Anomaly, Hypoplastic Left Heart Syndrome, Interrupted Aortic Arch, Pulmonary Atresia, Single Ventricle, Tetralogy of Fallot, Total Anomalous Pulmonary Venous Return, Tricuspid Atresia, Truncus Arteriosus, Ventricular Septal Defect, Polycystic kidney disease, Diabetes Insipidus, Goodpasture's Disease, IgA Vasculitis, IgA Nephropathy, Lupus Nephritis, Adult Nephrotic Syndrome, Childhood Nephrotic Syndrome, Hemolytic Uremic Syndrome, Medullary Sponge Kidney, Kidney dysplasia, Renal artery stenosis, Renovascular hypertension, Renal tubular acidosis, Alport syndrome, Wenger's granulomatosis, Alagille syndrome, Cystinosis, Fabry disease, Focal segmental glomerulosclerosis (FSGS), Glomerulonephritis, aHUS (atypical hemolytic uremic syndrome), Hemolytic uremic syndrome (HUS), Henoch-Schonlein purpura, IgA nephropathy (Berger's disease), Interstitial nephritis, Minimal change disease, Nephrotic syndrome, Thrombotic thrombocytopenic purpura (TTP), Granulomatosis with polyangiitis (GPA), Adult Still's disease, Agammaglobulinemia, Alopecia areata, Autoimmune angioedema, Autoimmune dysautonomia, Autoimmune encephalomyelitis, Autoimmune hepatitis, Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Bullous pemphigoid, Celiac disease, Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Coxsackie myocarditis, CREST syndrome, Dermatitis herpetiformis, Dermatomyositis, Devic's disease (neuromyelitis optica), Discoid lupus, Eosinophilic esophagitis (EoE), Eosinophilic fasciitis, Erythema nodosum, Essential mixed cryoglobulinemia, Giant cell arteritis (temporal arteritis), Giant cell myocarditis, Granulomatosis with Polyangiitis, Guillain-Barre syndrome, Hashimoto's thyroiditis, Henoch-Schonlein purpura (HSP), Herpes gestationis or pemphigoid gestationis (PG), Hypogammalglobulinemia, IgG4-related sclerosing disease, Immune thrombocytopenic purpura (ITP), Inclusion body myositis (IBM), Lambert-Eaton syndrome, Leukocytoclastic vasculitis, Linear IgA disease (LAD), Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Primary biliary cirrhosis, Primary sclerosing cholangitis, Progesterone dermatitis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Thyroid eye disease (TED), Alagille Syndrome, Alcohol-Related Liver Disease, Autoimmune Hepatitis, Biliary Atresia, Cirrhosis, Lysosomal Acid Lipase Deficiency (LAL-D), Newborn Jaundice, Non-Alcoholic Fatty Liver Disease, Non-Alcoholic Steatohepatitis, Primary Biliary Cholangitis (PBC), Progressive Familial Intrahepatic Cholestasis (PFIC), Surgical scars, Hypertrophic scars, Acute Lymphoblastic Leukemia (ALL), Acute Myeloid Leukemia (AML), Adrenocortical Carcinoma, Kaposi Sarcoma (Soft Tissue Sarcoma), AIDS-Related Lymphoma (Lymphoma), Primary CNS Lymphoma (Lymphoma), Anal Cancer, Childhood astrocytomas, Atypical teratoid/rhabdoid tumor, Basal cell carcinoma of the skin, Bile duct cancer, Bladder cancer, Ewing Sarcoma, Osteosarcoma, Malignant fibrous histiocytoma, Brain tumor, Breast cancer, Bronchial tumor, Burkitt Lymphoma, Carcinoid Tumor (Gastrointestinal), Cardiac (Heart) Tumors, Childhood, Medulloblastoma, Germ Cell Tumor, Primary CNS Lymphoma, Cervical Cancer, Cholangiocarcinoma, Chordoma, Chronic Lymphocytic Leukemia (CLL), Chronic Myelogenous Leukemia (CML), Chronic Myeloproliferative Neoplasms, Colorectal Cancer, Craniopharyngioma, Cutaneous T-Cell Lymphoma, Ductal Carcinoma In Situ (DCIS), Endometrial Cancer (Uterine Cancer), Ependymoma, Esophageal Cancer, Esthesioneuroblastoma (Head and Neck Cancer), Retinoblastoma, Fallopian Tube Cancer, Gallbladder Cancer, Gastric (Stomach) Cancer, Gastrointestinal Carcinoid Tumor, Gastrointestinal Stromal Tumors (GIST), Germ Cell Tumors, Childhood Extracranial Germ Cell Tumors, Extragonadal Germ Cell Tumors, Ovarian Germ Cell Tumors, Testicular Cancer, Gestational Trophoblastic Disease, Hairy Cell Leukemia, Head and Neck Cancer, Heart Tumor, HepatocellularCancer, Histiocytosis, Langerhans Cell, Hodgkin Lymphoma, Hypopharyngeal Cancer (Head and Neck Cancer), Intraocular Melanoma, Islet Cell Tumors, Kidney (Renal Cell) Cancer, Langerhans Cell Histiocytosis, Laryngeal Cancer (Head and Neck Cancer), Lip and Oral Cavity Cancer, Lung Cancer (Non-Small Cell, Small Cell, Pleuropulmonary Blastoma, and Tracheobronchial Tumor), Male Breast Cancer, Melanoma, Melanoma, Intraocular (Eye), Meningioma, Merkel Cell Carcinoma (Skin Cancer), Mesothelioma, Malignant, Metastatic Squamous Neck Cancer with Occult Primary (Head and Neck Cancer), Midline Tract Carcinoma, Multiple Endocrine Neoplasia Syndromes, Multiple Myeloma/Plasma Cell Neoplasms, Mycosis Fungoides, Myelodysplastic Syndromes, Myelodysplastic/Myeloproliferative Neoplasms, Neuroblastoma, Non-Hodgkin Lymphoma, Non-Small Cell Lung Cancer, Oral Cancer, Lip and Oral Cavity Cancer and Oropharyngeal Cancer (Head and Neck Cancer), Osteosarcoma and Undifferentiated Pleomorphic Sarcoma of Bone Treatment, Ovarian Cancer, Pancreatic Cancer, Pancreatic Neuroendocrine Tumors, Papillomatosis, Paraganglioma, Parathyroid Cancer, Penile Cancer, Pharyngeal Cancer, Pheochromocytoma, Pituitary Tumor, Plasma Cell Neoplasm/Multiple Myeloma, Pleuropulmonary Blastoma, Pregnancy and Breast Cancer, Primary Central Nervous System (CNS) Lymphoma, Primary Peritoneal Cancer, Prostate Cancer, Rectal Cancer, Renal Cell (Kidney) Cancer, Retinoblastoma, Ewing Sarcoma (Bone Cancer), Soft Tissue Sarcoma, Sezary Syndrome, Skin Cancer, Small Cell Lung Cancer, Small Intestine Cancer, Soft Tissue Sarcoma, Squamous Cell Carcinoma of the Skin, Squamous Neck Cancer with Occult Primary, Metastatic (Head and Neck Cancer), Stomach (Gastric) Cancer, T-Cell Lymphoma, Cutaneous—see Lymphoma (Mycosis Fungoides and Sezary Syndrome), Testicular Cancer, Nasopharyngeal Cancer, Oropharyngeal Cancer, Hypopharyngeal Cancer, Thymoma and Thymic Carcinoma, Thyroid Cancer, Tracheobronchial Tumors, Transitional Cell Cancer of the Renal Pelvis and Ureter, Urethral Cancer, Uterine Cancer, Endometrial, Uterine Sarcoma, Vaginal Cancer, Vulvar Cancer, and Wilms Tumor.

39. The method according to claim 38, wherein the disease is selected from the group consisting of brain tumor, gastric cancer, colorectal cancer, mesothelioma, non-small cell lung cancer, meningioma, head and neck cancer, and soft tissue sarcoma.

40. The method according to claim 38 or 39, further comprising administering at least one additional chemotherapeutic agent.

41. The method according to claim 40, wherein the additional chemotherapeutic agent is selected from a B-RAF inhibitor, epidermal growth factor receptor (EGFR) inhibitor, MEK inhibitor, and immune check point inhibitor.

42. The method according to claim 41, wherein the B-RAF inhibitor is selected from the group consisting of vemurafenib, dabrafenib, and encorafenib.

43. The method according to claim 41, wherein the MEK inhibitor is selected from the group consisting of trametinib, cobimetinib, selumetinib, and binimetinib.

44. The method according to claim 41, wherein the EGFR inhibitor is selected from the group consisting of erlotinib, osimertinib, neratinib, gefitinib, cetuximab, panitumumab, dacomitinib, lapatinib, necitumumab, mobocertinib, and vandetanib.

45. The method according to claim 41, wherein the immune check point inhibitor is selected from the group consisting of a PD-1 inhibitor and PD-L1 inhibitor.

46. The method according to claim 45, wherein the PD-1 inhibitor is selected from the group consisting of pembrolizumab, nivolumab, and cemiplimab.

47. The method according to claim 45, wherein the PD-L1 inhibitor is selected from the group consisting of atezolizumab, avelumab, and durvalumab.
